(19) United States
(12) Patent Application Publication
(10) Pub. No.: US 2014/0107100 A1
(43) Pub. Date: Apr. 17, 2014

(54) Benzoxazepines as Inhibitors of PI3K/mTOR and Methods of Their Use and Manufacture

(71) Applicant: EXELIXIS, INC., South San Fransisco, CA (US)

(72) Inventors: Kenneth Rice, San Rafael, CA (US); Paul Foster, Novato, CA (US)

(73) Assignee: EXELIXIS, INC., South San Fransisco, CA (US)

(21) Appl. No.: 13/989,156

(22) PCT Filed: Nov. 23, 2011
(86) PCT No.: PCT/US11/62052

§ 371 Date: Nov. 12, 2013

Related U.S. Application Data
(60) Provisional application No. 61/417,122, filed on Nov. 24, 2010.

(51) Int. Cl. C07D 513/04; C07D 413/06; C07D 417/14; C07D 413/14

(52) U.S. Cl. CPC C07D 513/04; C07D 413/14; C07D 413/06; C07D 417/14
USPC 514/211.09; 540/552

(57) ABSTRACT

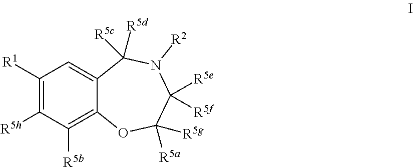

The invention is directed 10 Compound's of Formula I: and pharmaceutically acceptable salts or solvates thereof, as well as methods of making and using the compounds.

### CROSS-REFERENCE TO RELATED APPLICATIONS

This application claims the benefit of priority to U.S. Provisional Application No. 61/417,122, filed Nov. 24, 2010, which is incorporated herein by reference.

### SEQUENCE LISTING

This application incorporates by reference in its entirely the Sequence Listing entitled “10-025_Sequence.txt” (16.2 KB) which was created Nov. 23, 2011 and filed herewith on Nov. 23, 2011.

### BACKGROUND OF THE INVENTION

1. Field of the Invention

This invention relates to the field of protein kinases and inhibitors thereof. In particular, the invention relates to inhibitors of PI3K and/or the mammalian target of rapamycin (mTOR) signaling pathways, and methods of their use and preparation.

2. Background of the Invention

The PI3K pathway regulates cell growth, proliferation and survival, and is deregulated with high frequency in human minors. PI3K pathway activation in tumors occurs via multiple mechanisms including prevalent mutation and amplification of the PIK3CA gene (which encodes the p1 10 summit of PI3Ka), or dow-nregulation of the lipid phosphatase PTEN. Downstream of PI3K. mTOR controls cell growth ami proliferation through its two distinct signaling complexes: mTORC1 and mTORC2. Given the role of PI3K signaling on critical cellular functions, an inhibitor that targets both PI3K and mTOR could provide therapeutic benefit to patient populations with tumors harboring activating mutations in PIC3CA or Ras. PTEN-deletion, or where tumors are upregulated in growth factor signaling.

Recent studies indicate that phosphatidylinositol 3-kinase (PI3K) signaling has significant effects on cancer cell growth, survival, motility, and metabolism. The PI3K pathway is activated by several different mechanisms in cancers, including somatic mutation and amplification of genes encoding key components. In addition. PI3K signaling may serve integral functions for noncancerous cells in the tumor microenvironmcni. Consequently, there is continued interest in developing inhibitors of PI3K isoforms as a means for treating various forms of cancer, particularly the class II isoforms PI3K-alpha, PI3K-beta. and PI3K-gamma.

For example, phosphatidylinosital 3-kinase (PI3Kα), a dual specificity protein kinase, is composed of an 85 kDa regulatory subunit and a 110 kDa catalytic subunit. The protein encoded by this gene represents the catalytic subunit, which uses ATP in phosphorylale Ptdlns. Ptdllns4P and Ptdlns(4.5)P2. PTEN. a tumor suppressor which inhibits cell growth through multiple mechanisms, can dephosphorylaic PIP3, the major product of PIK3CA. PIP3, in turn, is required for translocation of proicin kinase B (AKT1, PKB) to the cell membrane, where it is phosphorylaied and activated by upstream kinases. The effect of PTEN on cell death is mediated through ihc PIK3CA/AKT1 pathway.

PI3Kα has been implicated in the control of cyloskeletal reorganization, apopiosis, vesicular trafficking, proliferation and differentiation processes. Increased copy number and expression of PIK3CA is associated with a number of malignancies such as ovarian cancer (Campbell et al.. Cancer Res 2004, 64, 7678-7681: Levine et al., Clin Cancer Res 2005. 11. 2875-2878; Wang el al., Hum Mutat 2005, 25, 322; Lee el al., Gynecol Oncol 2005, 97, 26-34). cervical cancer, breast cancer (Bachman. et al, Cancer Biol Ther 2004, 3, 772-775; Levine, et al., supra; Li el al., Breast Cancer Res Treat 2006, 96, 91-95; Saal et al., Cancer Res 2005, 65, 2554-2559; Samuels and Velculescu, Cell Cycle 2004, 3, 1221-1224), colorectal cancer (Samuels, et al. Science 2004, 304, 554; Velho et al. Eur J Cancer 2005, 41, 1649-1654), endometrial cancer (Oda et al. Cancer Res. 2005, 65, 10669-10673), gastric carcinomas (Byun et al., Int J Cancer 2003, 104, 318-327; Li et al., supral; Velho et al., supra; Lee et al., Oncogene 2005, 24, 1477-1480), hepatocellular carcinoma (Lee et al., id.), small and non-small cell lung cancer (Tang et al., Lung Cancer 2006, 51, 181-191; Massion et al., Am J Respir Crit Care Med 2004, 170, 1088-1094), thyroid carcinoma (Wu et al., J Clin Endocrinol Metab 2005, 90, 4688-4693), acute myelogenous leukemia (AML) (Sujobert et al., Blood 1997, 106, 1063-1066), chronic myelogenous leukemia (CML) (Hickey and Cotter J Biol Chem 2006, 281, 2441-2450), and glioblastomas (Hartmann et al.Aeta Neuropathol (Bert) 2005, 109, 639-642; Samuels et al., supra).

The mammalian target, mTOR, is a protein kinase that integrates both extracellular and intracellular signals of cellular growth, proliferation, and survival. Extracellular mitogenic growth factor signaling from cell surface receptors and intracellular pathways that convey hypoxic stress, energy and nutrient status all converge at in TOR. mTOR exists in two distinct complexes: mTOR complex 1 (mTORC1) and mTOR complex 2 (mTORC2). mTORC1 is a key mediator of transcription and cell growth (via its suhstrates p70S6 kinase and 4E-BP1) and promotes cell survival via the serum and glueocorticoid-activatcd kinase SGK, whereas mTORC2 promotes activation or the pro-survival kinase AKT. Given its central role in cellular growth, proliferation and survival, it is perhaps not surprising that mTOR signaling is frequently dysregulated in cancer and other diseases (Bjornsti and Houghton Rev Cancer 2004, 4(5), 335-48: Houghton and Huang Microbiol Immunol 2004, 279, 339-59: Inoki. Corradetti et al, Nat Genet 2005, 37(1), 19-24).

mTOR is a member of the PIKK (PI3K-related Kinase) family of atypical kinases which includes ATM, ATR, and DNAPK, and its catalytic domain is homologous to that of PI3K. Dyregulalion of PI3K signaling is a common function of tumor cells. In general, mTOR inhibition may be considered as a strategy in many of the lumor lypes in which PI3K signaling is implicated such as those discussed below.

Inhibitors of mTOR may be useful in treating a number of cancers, including ihc following: breast cancer (Nagata, Lan et al., Cancer Cell 2004, 6(2), 117-27; Pandolfi N Engl J Med 2004, 351(22), 2337-8; Nahla. Yu et al. Nat Clin Pract Oncol 2006,3(5), 269-280); antle cell lymphoma (MCL) (Dal Col. Zancai et al. Blood 2008., 111(10), 5142-51); renal cell carcinoma (Thomas, Tran et al. Nat Med 2006, 12(1), 122-7; Atkins, Hidalgo et at J Clin Oncol 2004, 22(5), 909-18; Motzer. Hudes et al. J Clin Oncol 2007,25(25), 3958-64); acute myelogenous leukemia (AML) (Sujobert, Bardet et al. Blood 2005, 106(3), 1063-6; Billottet, Grandage et al. Oncogene 2006, 25(50), 6648-6659; Tamburini, Elie et al. Blood 2007, 110(3), 1025-8); chronic myelogenous leukemia (CML) (Skorski, Bellacosa et al. Embo J 1997, 16(20), 6151-61; Bai. Ouyang et al. Blood 2000, 96(13), 4319-27; Hickey and Cotter Biol Chem 2006, 281(5), 2441-50); diffuse large B cell lymphoma (DLBCL) (Uddin, Hussain et al. Blood, 2006, 108(13), 4178-86); several subtypes of sarcoma (Hernando, Charytonowiez et al. Nat Med 2007, 13(6), 748-53; Wan and Helman Oncologist 2007, 12(8), 1007-18); rhabdomyosarcoma (Cao. Yu et al. Cancer Res 2008, 6S(19), 8039-8048; Wan. Shen et al. Neoplasia 2006, 8(5), 394-401); ovarian cancer (Shayesteh, Lu et al. Nat Genet, 1999, 21(1). 99-102; (Lee, Choi et al. Gynecol Oncol 2005, 97(1) 26-34); endometrial tumors (Obata. Morland et al. Cancer Res 1998, 58(10), 2095-7; Lu. Wu et al. Clin Cancer Res 2008, 14(9). 2543-50); non small cell lung carcinoma (NSCLC) (Tang, He et al. Lung Cancer 2006, 51(21), 181-91; Marsit, Zheng et al. Hum Pathol 2005, 36(7). 768-76); small cell, squamous, large cell and adenocarcinoma (Massion, Taflan et al. Am J Respir Crit Care Med 2004, 170(10). 1088-94); lung tumors in general (Kokubo, Gemma et al. Br J Cancer 2005, 92(9), 1711-9: Pao. Wang et al. Pub Library of Science Med 2005, 2(1), e17); colorectal tumors (Velho. Oliveira et al. Eur J Cancer 2005, 41(11). 1649-54; Foukas, Claret et al. Nature, 2006, 441(7091), 366-370), particularly those that display microsatellite instability (Goel, Arnold et al. Cancer Res 2004, 64(9), 3014-21; Nassif, Lobo et al. Oncogene 2004, 23(2), 617-28), KRAS-mutated colorectal tumors (Bos Cancer Res 1989, 49(17), 4682-9; Fearon Ann N.Y. Acad Sci 1995, 70S. 101-10); gastric carcinomas (Byun, Cho et al. Int J Cancer 2003, 104(3), 318-27); hepatocellular minors (Lee, Soung et al. Oncogene 2005, 24(8), 1477-80); liver tumors (Hu, Huang el al. Cancer 2003, 97(8), 1929-40; Wan, Jiang et al. Cancer Res Clin Oncol 2003, 129(2), 100-6); primary melanomas and associated increased tumor thickness (Guldberg, thor Straten et al. Cancer Res 1997, 57(17), 3660-3; Tsao. Zhang et al. Cancer Res 2000, 60(7), 1800-4; Whiteman, Zhou et al. Int J Cancer 2002, 99(1), 63-7; Goel. Lazar et al. J Invest Dermatol 126(1), 2006, 154-60); pancreatic tumors (Asano. Yao et al. Oncogene 2004, 23(53), 8571-80); prostate carcinoma (Cairns, Okami et al. Cancer Res 1997, 57(22), 4997-5000; Gray, Stewart et al. Br J Cancer 1998, 78(10), 1296-300; Wang, Parsons et al. Clin Cancer Res 1998, 4(3), 811-5; Whang, Wu et al. Proc Natl Acad Sci USA 1998, 95(9), 5246-50; Majumder and Sellers Oncogene 2005, 24(50) 7465-74; Wang. Garcia et al. Proc Natl Acad Sci U S A 2006, 103(5), 1480-5; (Lu. Ren et al. Int J Oncol 2006, 28(1), 245-51; Mulholland. Dedhar et al. Oncogene 25(3), 2006, 329-37: Xin. Teitell et al. Proc Natl Acad Sci USA 12006, 03(20), 7789-94; Mikhailova. Wang et al. Adv Exp Med Biol 2008, 617. 397-405: Wang. Mikhailova et al. Oncogene 2008, 27(56). 7106-7117); thyroid carcinoma, particularly in the anaplastic subtype (Garcia-Rostan. Costa et al. Cancer Res 2005, 65(22). 10199-207); follicular thyroid carcinoma (Wu. Mamho et al. J Clin Endocrinol Metab 2005, 90(8), 4688-93): anaplastic large cell lymphoma (ALCL): hamaratomas, angiomyelolipomas. TSC-associated and sporadic lymphangioleiomyomatosis: Cowdcn's disease (multiple hamaratoma syndrome) (Bissler, McCormack et al. N Engl J Med 2008, 358(2). 140-151); sclerosing hemangioma (Randa M. S. Amin Pathology International 2008, 58(1). 38-44): Pcuix-Jcghcrs syndrome (PJS); head and neck cancer (Gupta, McKcrma et al. Clin Cancer Res 2002, 8(3). 885-892): neurofibromatosis (Ferner Eur J Hum Genet 2006, 15(2). 131-138: Sabatini Nat Rev Cancer 2006, 6(9). 729-734: Johannessen. Johnson et al. Current Biology 2008, 18(1). 56-62): macular degeneration: macular edema; myeloid leukemia: systemic lupus: and autoimmune lymphoproliferative syndrome (ALPS).

### SUMMARY OF THE INVENTION

The following only summarizes certain aspects of the invention and is not intended to be limiting in nature. These aspects and other aspects and embodiments are described more fully below. All references cited in this specification are hereby incorporated by reference in their entirety. In the event of a discrepancy between the express disclosure of this specification and the references incorporated by reference, the express disclosure of this specification shall control.

We recognized the important role of PI3K and mTOR in biological processes and disease states and, therefore, realized that inhibitors of these protein kinases would be desirable, as evidenced in Serial Number PCT/US2010/036032, filed May 25, 2010, the entire contents of which is incorporated herein by reference. Accordingly, the invention provides compounds that inhibit, regulate, and/or modulate PI3K and/or mTOR and are useful in the treatment of hyperproliferativc diseases, such as cancer, in mammals. This invention also provides methods of making the compound, methods of using such compounds in the treatment of hyperproliferative diseases in mammals, especially humans, and to pharmaceutical compositions containing sch compounds.

A first aspect of the invention provides a Compound of Formula I:

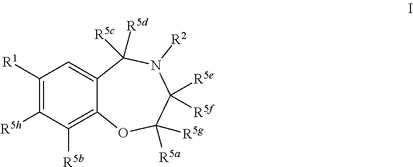

or a single stereoisomer or mixture of stereoisomers thereof and additionally optionally as a pharmaceutically acceptable salt thereof, where
R¹ is phenyl optionally substituted with one, two, or three R⁶ groups; or
R¹ is heteroaryl optionally substituted with one, two, or three R⁷;
R² is heteroaryl substituted with R³, R^{3a}, R^{3b}, R^{3c} , and R^{3d};
R³, R^{3a}, R^{3b},R^{3c}, and R^{3d}are independently hydrogen, cyano, nitro, alkyl, alkenyl, alkynyl, halo, haloalkyl, hydroxyalkyl, alkoxyalkyl, cyanoalkyl, —SR¹³, —S(O)₂R²⁰, —C(O)H, —C(O)OR⁴, —C(O)NHR⁴, halocarbonyl, —NR¹¹R^{11a}, —OR^{11a}, optionally substituted phenyl, optionally substituted phenylalkyl, optionally substituted cycloalkyl, optionally substituted cycloalkylalkyl, optionally substituted heterocycloalkyl, optionally substituted hcterocycloalkylalkyl,optionally substituted heteroaryl, optionally substituted heteroarylalkyl, or alkyl substituted with one or two R¹⁶; or
two of R³, R^{3a}, R^{3c}, and R^{3d}, when attached to the same carbon, form an optionally substituted cycloalkyl, optionally substituted aryl, or an optionally substituted heterocycoalkyl, or optionally substituted heteroaryl, and the other of R³, R^{3a}, R^{3b}, R^{3c}, and R^{3d} are independently hydrogen, cyano, nitro, alkyl, alkenyl, alkynyl, halo, haloalkyl, hydroxyalkyl, alkoxyalkyl, cyanoalkyl, —SR¹¹, —S(O)₂R²⁰, —C(O)H, —C(O)OR⁴, halocarbonyl, —C(O)NNR⁴, halocarbonyl, —NR¹¹RR^{11a}, —OR^{11a} optionally substituted phenyl, optionally substitutedd phenylalkyl, optionally substitutedd cycloalkyl, optionally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶;
R⁴ is alkyl, alkenyl, alkynyl, hydroxyalkyl, alkoxyalkyl, haloalkyl, aminoalkyl, alkylaminoalkyl, dialkylaminoalkyl, benzyl, or optionally substituted heterocycloalkylalkyl;
R^{5a} and R^{5c} are independcnily hydrogen, deuterium, or alkyl;
R^{5h} is hydrogen, deuterium or halo;
R^{5b} is deuterium, (C₁₋₃)alkyl, (C₁₋₃)alkoxy, halo(C₁₋₃)alkyl, or (C₁₋₃)haloalkoxy;
R^{5d}, R^{5e}, R^{5f}, and R^{5g} are hydrogen or deuterium;
each R⁶, when R⁶ is present, is independently nitro; cyano; halo; alkyl; alkenyl; alkynyl; haloalkyl; —OR^{8a}, —NR⁸R^{8a}; —C(O)NR⁸R^{8a}; —S(O)₂R⁸; —NR⁸C(O)OR⁹; —NR⁸C(O)R⁹; —NR⁸S(O)₂R^{8a}; —NR⁸C(O)NR^{8a}R⁹; carboxy. —C(O)OR⁹; halocarbonyl; alkylcarbonyl; alkyl substituted with one or two —C(O)NR⁸R^{8a}; heteroaryl optionally substituted with 1, 2, or 3 R¹⁴; or optionally substituted heterocycloalkyl; or
two R⁶, together with the carbons to which they are attached, form an optionally substituted 3, 4, 5, or 6-membered cycloalkyl or heterocycloalkyl;
each R⁷, when R⁷ is present, is independcnily oxo; nitro; cyano; alkyl; alkenyl; alkynyl; halo; haloalkyl; hydroxyalkyl: alkoxyalkyl; —OR^{8a}; —SR¹³; —S(O)R¹³; —S(O)₂R^{13a}; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NR^{8a}R⁹; —C(O)OR^{8a}; halocarbonyl; alkylcarbonyl; —S(O)₂NR⁸R⁹; alkylsulfonylakyl; alkyl substituted with one or two —NR⁸R^{8a}; alkyl substituted with one or two —NR⁸C(O)R^{8a}; alkyl substituted with one or two —NR⁸C(O)OR⁹; alkyl sustituted with one or two —S(O)₂S^{13a}; optionally substitutedd cycloalkyl; optionally substituted cycloalkylalkyl; optionally suubstituted heterocycloalkyl; opiionally substituted heterocycloalkylalkyl; optionally substituted phenyl; optionally substituted phenylalkyl; optionally substituted heteroaryl; or optionally substituted heteroarylalkyl;
each R⁸, R¹¹, R¹⁵, R¹⁷, and R¹⁸ are independcnily hydrogen, NH₂, NH(alkyl), N(alkyl)₂, alkyl, alkenyl, alkynyl, hydroxyalkyl, alkoxyalkyl, or haloalkyl;
each R^{8a}, R^{11a}, and R^{15a} are independently hydrogen, alkyl, alkenyl, alkynyl, haloalkyl, hydroxyalkyl, cyanoalkyl, aminoalkyl, alkylaminoalkyl, dialkylaminoalkyl, alkoxyalkyl, carboxyalkyl, optionally substitutedd cycloalkyl, optionally substituted cycloalkylalkyl, optionally substituted heterocycloalkyl, optionally substituted hetecrocycloalkylalkyl, optionally substituted phenyl, optionally substituted phenylalkyl, optionally substituted heteroaryl, or optionally substituted heteroarylalkyl;
R⁹ is hydrogen; alkyl; alkenyl; alkynyl; hydroxyalkyl; alkoxyalkyl; anunoalkyl; alkylaminoalkyl; dialkylaminoalkyl; haloalkyl; hydroxyalkyl substituted with one. two. or three groups which are independently halo, amino, alkylamino, or dialkylamino; alkyl substituted with one or two aminocarbonyl; optionally substituted phenyl; optionally substituted phenylalkyl; optionally substituted cycloalkyl; optionally substituted cyeloalkylalkyl; optionally substituted heteroaryl; optionally substituted heteroarylalkyl; optionally substituted heterocycloalkyl; or optionally substituted heterocycloalkylalkyl;
R¹² is alkyl or optionally substituted phenylalkyl;
R¹³ is alkyl, hydroxyalkyl, or haloalkyl; and
R^{13a} is hydroxy, alkyl, haloalkyl, hydroxyalkyl, or heterocycloalkyl optionally substituted with one or two groups which are independently halo, amino, alkylamino, dialkylamino, hydroxy, alkyl, or hydroxyalkyl;
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarhonyl, aminocarbonyl, alkylaminocarbonyl, dialkylaminocarbonyl, or optionally substituted phenyl;
each R¹⁶ is independently halo, —NR¹¹R^{11a}, —NR¹⁵S(O)R^{15a}, —OC(O)R¹⁷, carboxy, alkoxycarbonyl, —NHC(O)R^{15a}, or —OR¹⁸; and
R₂₀ is alkyl, haloalkyl, hydroxyalkyl, amino, alkylamino, dialkylamino, or heterocycloalkyl;
   with the proviso that if one of R^{5a}, R^{5c}, R^{5d}, R^{5e}, R_{f}, R^{5g}, and R^{5h} are deuterium, then R^{5b} is H, (C₁₋₃)alkyl or halo(C₁₋₃)alkyl.

In a second aspect, the invention is directed to a pharmaceutical composition which comprises 1) a Compound of Formula I or a single stereoisomer or mixture of stereoisomers thereof, optionally as a pharmaceutically acceptable salt or solvate thereof and 2) a pharmaceutically acceptable carrier, excipient, or diluent.

In a third aspect of the invention is a method of inhibiting the in vivo activity of PI3K and/or mTOR. the method comprising administering to a patient an effective PI3K-inhibiting and/or mTOR-inhibiting amountof a Compound of Formula Ia Compound of Formula I or a single stereoisomer or mixture of stereoisomers thereof, optionally as a pharinaccutically acceptable salt or solvate thereof or pharmaceutical composition thereof.

In a fourth aspect, the invention provides a method for treating a disease, disorder, or syndrome which method comprises administering to a patient a therapeutically effective amount of a Compound of Formula I or a single stereoisomer or mixture of stereoisomers thereof, optionally as a pharmaceutically acceptable salt or solvate thereof, or a pharmaceutical composition comprising a therapeutically effective amount of a Compound of Formula I or a single stereoisomer or mixture of stereoisomers thereof, optionally as a pharmaceutically acceptable salt or solvate thereof, and a pharmaceutically acceptable carrier, excipicnt, or diluent.

In a fifth aspect, the invention provides a method for making a Compound of Formula I(a) which method comprises
(a) reacting the following, or a salt thereof:

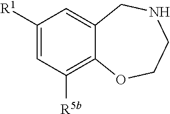

where R¹ is as defined in the Summary of the Invention for a Compound of Formula I; with an intermediate of Formula R²X where X is halo, and R² is as defined in the Summary of the Invention for a Compound of Formula I to yield a Compound of ihc Invention of Formula I(a)

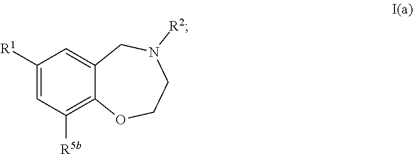

and optionally separating individual isomers; and optionally modifying any of the R¹ and R² groups; and optionally forming a phannaceutically acceptable salt thereof; or
(b) reacting the following, or a salt thereof:

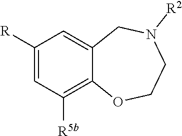

where R is halo or —B(OR′)₂ (where both R′ are hydrogen or the two R′ together form a boronk ester), and R² is as defined in the Summary of the Invention for a Compound of Formula I: with an intermediate of Formula R¹Y where Y is halo when R is —B(OR′)₂ and Y is —B(OR′)₂ when R is halo, and R² is as defined in the Summary of the Invention for a Compound of Formula I to yield a Compound of the Invention of Formula I(a): and optionally separating individual isomers: and optionally modifying any of the R¹ and R² groups; and optionally forming a pharmaceutically acceptable salt, hydrate, solvate or combination thereof.

In an additional aspect of the invention is a method of inhibiting the in vivo activity of mTOR, the method comprising administering to a patient an effective PI3K/mTOR-inhibiting amount of a compound of formula I or of Table 1 or a single stereoisomer or mixture of isomers thereof, optionally as a pharmaceutically acceptable salt or solvate thereof or pharmaceutical composition thereof. In this and other aspects and embodiments as provided herein, the compound can be an inhibitor of PI3Kα, PI3Kβ, PI3Kγ, or other PI3K isoforms combinations thereof.

In an additional aspect of the Invention provides a method for treating a disease, disorder, or syndrome which method comprises administering to a patient a therapeutically effective amount of a compound of formula I or a single stereoisomer or mixture of isomers thereof, optionally as a pharmaceutically acceptable salt or solvate thereof, or a pharmaceutical composition comprising a therapeutically effective amount of a compound of formula I or of Table 1 or a single stereoisomer or mixture of isomers thereof, optionally as a pharmaceutically acceptable salt or solvate thereof, and a pharmaceutically acceptable carrier, excipient, or diluent.

In an additional aspect of the invention provides a method for treating a subject having a tumor the method comprising: (a) administering a PI3K-α selective inhibitor, a dual PI3K-α/mTOR selective inhibitor, or a combination of a PI3K-α selective inhibitor and a mTOR selective inhibitor to the subject if said tumor comprises a mutation in a PI3K-α kinase domain: or (b) administering a combination of a PI3K-α selective inhibitor and a PI3K-α selective inhibitor, a dual PI3K-α/mTOR selective inhibitor, or a PI3K-β selective inhibitor, to said subject if said tumor comprises a mutation in a PI3K-α helical domain, wherein the PI3K-α selective inhibitor, the dual PI3K-α/mTOR selective inhibitor, or the combination of the PI3K-α selective inhibitor and a mTOR selective inhibitor is a compound of Formula I or of Table 1.

In an additional aspect, the present invention provides a method for identifying a selective inhibitor of a PI3K isozyme, the method comprising: (a) contacting a first cell hearing a first mutation in a PI3K-α with a candidate inhibitor: (b) contacting a second cell bearing a wild type PI3K-α, a PTEN null mutation, or a second mutation in said PI3K-α with the candidate inhibitor, and (c) measuring AKT phosphorylation in said first and said second cells, wherein decreased AKT phosphorylation in said first cell when compared to said second cell identifies said candidate inhibitor as a selective PI3K-α inhibitor, wherein the PI3K-α selective inhibitor, the dual PI3K-α/mT0R selective inhibitor, or the combination of the PI3K-α selective inhibitor and a mTOR selective inhibitor is a compound of Formula I or of Table 1.

In an additional aspect, the present invention provides for a method for determining a treatment regimen for a cancer patient having a tumor comprising a PI3K-α, the method comprising: determining the presence or absence of a mutation in amino acids 1047 and/or 545 of said PI3K-α; wherein if said PI3K-α has a mutation at position 1047, said method comprises administering to the cancer patient a therapeutically effective amount of a PI3K-α selective inhibitor compound, or a dual PI3K-α/mTOR selective inhibitor, or a combination of a PI3K-α selective inhibitor and a mTOR selective inhibitor; or wherein if said PI3K-α has a mutation at position 545, said method comprises administering to the cancer patient a therapeutically effective amount of a combination of a PI3K-α selective inhibitor and a PI3K-β selective inhibitor, or a dual PI3K-α/mTOR selective inhibitor, or a combination of a PI3K-α selective inhibitor and a mTOR selective inhibitor; wherein the PI3K-α selective inhibitor, the dual PI3K-α/mTOR selective inhibitor, or the combination of the PI3K-α selective inhibitor and a mTOR selective inhibitor is a compound of Formula I or of Table 1.

In an additional aspect, the cell used to diagnose, treat or screen against includes a cancer or tumor cell obtained from a tumor or cancer derived from: breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non-small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer, wherein the PI3K-α selective inhibitor, the dual PI3K-α/mTOR selective inhibitor, or the combination of the PI3K-α selective inhibitor and a mTOR selective inhibitor is a compound of Formula I or of Table 1.

### DETAILED DESCRIPTION OF THE INVENTION

### Abbreviations and Definitions

The following abbreviations and terms have the indicated meanings throughout:

| Abbreviation | Meaning |
|---|---|
| br | broad |
| ° C. | degrees Celsius |
| d | doublet |
| dd | doublet of doublet |
| dt | doublet of triplet |
| DCM | dichloromethane |
| DIEA or DIPEA | N,N-di-isopropyl-N-ethylamine |
| DMA | N,N-dimethylacetamide |
| DME | 1,2-dimethoxyethane |
| DMF | N,N-dimethylformamide |
| DMSO | dimethyl sulfoxide |
| dppf | 1,1′-bis(diphenylphosphano)ferrocene |
| El | Electron Impact ionization |
| g | gram(s) |
| GC/MS | gas chromatography/mass spectrometry |
| h or hr | hour(s) |
| HPLC | high pressure liquid chromatography |
| L | liter(s) |
| LC/MS | liquid chromatography/mass spectrometry |
| M | molar or molarity |
| m | Multiplet |
| MeOH | methanol |
| mg | milligram(s) |
| MHz | megahertz (frequency) |
| min | minute(s) |
| mL | milliliter(s) |
| μL | microliter(s) |
| μM | micromolar |
| μmol | micromole(s) |
| mM | Millimolar |
| mmol | millimole(s) |
| mol | mole(s) |
| MS | mass spectral analysis |
| N | normal or normality |
| nM | nanomolar |
| NMP | N-methyl-2-pyrrolidone |
| NMR | nuclear magnetic resonance spectroscopy |
| q | Quartet |
| rt | Room temperature |
| s | Singlet |
| t or tr | Triplet |
| THF | tetrahydrofuran |

The symbol “−” means a single bond, “=” means a double bond, “≡” means a triple bond, [symbol] 0 means a single or double bond. The symbol [symbol] refers to a group on a double-bond as occupying either position on the terminus of a double bond to which the symbol is attached: that is, the geometry, E- or Z-, of the double bond is ambiguous. When a group is depicted removed from its parent Formula, the “˜” symbol will be used at the end of the bond which was theoretically cleaved in order to separate the group from its parent structural Formula.

When chemical structures arc depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual Formula as having a hydrogen or hydrogens as substitution (expressly defined hydrogen), for example. “CH₂CH₂—. It is understood by one of ordinary skill in the art that the aforementioned descriptive techniques are common in the chemical arts to provide brevity and simplicity to description of otherwise complex structures.

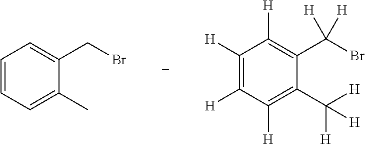

If a group “R” is depicted as “floating” on a ring system, as for example in the Formula:

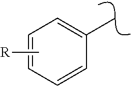

then, unless otherwise defined, a substiiuem “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the Formula e:

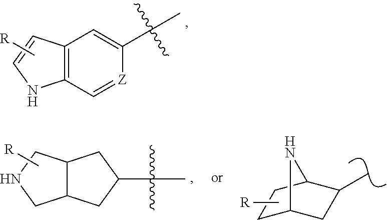

then, unless otherwise defined, a substiiuent “R” may reside on any atom of the fused ring system, assuming replacement of a depicted hydrogen (for example the —NH— in the Formula above), implied hydrogen (for example as in the Formula above, where the hydrogens are not shown but understood to be present), or expressly defined hydrogen (for example where in the Formula above, “Z” equals ═CH—) from one of the ring atoms, so long as a stable structure is formed. In the example depicted, the “R” group may reside on either the 5-membered or the 6-membered ring of the fused ring system.

When a group “R” is depicted as existing on a ring system containing saturated carbons, as for example in the Formula:

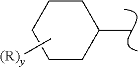

where, in this example, “y” can be more than one, assuming each replaces a currently depicted, implied, or expressly defined hydrogen on the ring; then, unless otherwise defined, where the resulting structure is stable, two “R's” may reside on the same carbon. In another example, two R's on the same carbon, including that carbon, may form a ring, thus creating a spirocyclic ring structure with the depicted ring as for example in the Formula:

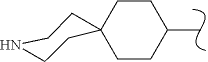

“Acyl” means a “C(O)R radical where R is alkyl, alkenyl, cycloalkyl, cycloalkylalkyl, aryl, aralkyl, heteroaryl, hetcroaralkyl, heterocycloalkyl, or heterocycloalkylalkyl, as defined herein, e.g., acetyl, trifluoromethylcarbonyl, or 2-methoxycehylcarbonyl, and the like.

“Acylamino” means a —NKR′ radical where R is hydrogen, hydroxy, alkyl, or alkoxy and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a Compound of the invention means introducing ihe Compound or a prodrug of the Compound into the sysiem of the animal in need of treatment. When a Compound of the invention or prodrug thereof is provided in combination with one or more other active agents (e.g., surgery, radiation, and chemotherapy, etc.), “adminisiraiion” and its variants are each understood to include concurrent and sequential introduction of the Compound or prodrug thereof and other agents.

“Alkenyl” means a means a linear monovalent hydrocarbon radical of two to six carbon atoms or a branched monovalent hydrocarbon radical of three to six carbon atoms which radical contains at least one double bond. e.g., ethenyl, propcnyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, specifically one, two, or three, alkoxy groups as defined herein. Representative examples include methoxymethyl and the like.

“Alkoxycarbonyl” means a —C(O)R group where R is alkoxy, as defined herein.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylaminoalkyloxy” means an —OR group where R is alkyiaminoalkyl, as defined herein.

“Alkylcarbonyl” means a —C(O)R group where R is alkyl, as defined herein.

“Alkylsulfonyl” means an —S(O)₂R group where R is alkyl, as defined herein.

“Alkylsulfonylalkyl” means an alkyl group, as defined herein, substituted with one or iwo —S(O)₂R group where R is alkyl, as defined herein.

“Alkynyl” means a linear monovalent hydrocarbon radical of two to six carbon aioms or a branched monovalent hydrocarbon radical of three to 6 carbon atoms which radical contains at least one triple bond, e.g., ethynyl, propynyl, butynyl, pentyn-2-yl and the like.

“Amino” means —NH₂.

“Aminoalkyl” means an alkyl group subsiiuied with at least one, specifically one, two or three, amino groups.

“Aminoalkyloxy” means an —OR group where R is aminoalkyl, as defined herein.

“Aminocarbonyl” means a —C(O)NH₂ group.

“Alkylaminocarbonyl” means a —C(O)NHR group where R is alkyl as defined herein.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical. valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Cyanoalkyl” means an alkyl group, as defined herein, substituted with on or two cyano groups.

“Cycloalkyl” means a monocyclic or fused bicyclic, saturated or partially unsaturated (but not aromatic), monovalent hydrocarbon radical of three to ten carbon ring atoms. Fused bicyclic hydrocarbon radical includes spiro and bridged ring systems. Unless stated otherwise, the valency of the group may be located on any atom of any ring within the radical, valency rules permitting. One or two ring carbon atoms may be replaced by a —C(O)- , —C(S)—, or —C(═NH)- group. More specifically, the term cycloalkyl includes, but is not limited to, cyclopropyl, cyclobutyl, cyclopentyl, cyclohexyl, cyclohexyl, or cyclohex-3-enyl, and the like.

“Cycloalkylalkyl” means an alkyl group substituted with at least one, specificallyone or two, cycloalkyl group(s) as defined herein.

“Dialkylamino” means an —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with one or two dialkylamino groups, as defined herein.

“Dialkylaminoalkyloxy” means an —OR group where R is dialkylaminoalkyl, as defined herein. Representative examples include 2-(N,N-dieihylamino)-ethyloxy, and the like.

“Dialkylaminocarbonyl” means a —C(O)NRR′ group where R and R′ are alkyl as defined herein.

“Fused ring system” means a polycyclic ring system that contains bridged or fused rings; that is, where two rings have more than one shared atom in their ring structures. In this application, fused ring systems are not necessarily all aromatic ring systems. Typically, but not necessarily, fused ring systems share a vicinal set of atoms, for example naphthalene or 1,2,3,4-tetrahydro-naphthalene. Fused ring systems of the invention may themselves have spiro rings attached thereto via a single ring atom of the fused ring system. In some examples, as appreciated by one of ordinary skill in the art, two adjacent groups on an aromatic system may be fused together to form a ring structure. The fused ring structure may contain heteroatoms and may be optionally substituted with one or more groups;:

“Halogen” or “halo” refers to fluorine, chlorine, bromine and iodine.

“Haloalkoxy” means an —OR′ group where R′ is haloalkyl as defined herein, e.g., trifluoromethoxy or 2,2,2-trifluoroelhoxy, and the like.

“Haloalkyl” mean an alkyl group substituted with one or more halogens, specifically 1,2,3,4,5, or 6 halo atoms, e.g., trifluoromethyl, 2-chloroethyl, and 2,2-difluoroethyl, and the like.

“Halocarbonyl” means a —C(O)X group where X is halo.

“Heteroaryl” means a monocyclic or fused bicyclic or tricyclic monovalent radical of 5 to 14 ring atoms containing one or more, specifically one, two, three, or four ring heteroatoms where each heteroatom is independently —O—, —S(O)ₙ—(n is 0, 1, or 2). —N═,—NH—. or N-oxidc, with the remaining ring atoms being carbon, wherein the ring comprising a monocyclic radical is aromatic and wherein at least one of the (used rings comprising the bicyclic radical is aromatic. One or two ring carbon atoms of any nonaromalic rings comprising a bicyclic radical may be replaced by a —C(O)—, —C(S)—, or —C(═NH)- group. Fused bicyclic radical includes bridged ring systems. Unless stated otherwise, the valency may be located on any atom of any ring of the hetcroaryl group, valency rules permitting. When the point of valency is located on the nitrogen, R8 is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl. 2,3-dihydro-1H-indolyl (including, for example, 2,3-dihydro-1H-indol-2-yl or 2,3-dihydro-1H-indolo-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, 5,6,7,8-tetrahydroquinazolinyl, quinoxalinyl, tetrazoyl, pyrazolyl, pyrazinyl, pyrimidinyl, pyridazinyl. oxazolyl, isooxazolyl, oxadiazolyl, benzoxazolyl, quinolinyl, 5,6,7,8-tetrahydroquinolinyl, isoquinolinyl, tetrahydroisoquinolinyl (including, for example, tetrahydroisoquinolin-4-yl or tetrahydroisoquiuolin-6-yl, and the like), pyrrolo[3,2-c]pyrindinyl (including, for example, pyrrolo[3,2-c]pyridin-2-yl or pyrrolo[3,2-c]pyridin-7-yl, and the like), benzopyranyl, 2.3-dihydrobenzofuranyl, benz[d][1.3]dioxolyl, 2.3-dihydrobenzo[b][1,4]dioxinyl, thiazolyl, isothiazolyl, thiadiazolyl, benzothiazolyl, benzothienyl, 6,7-dihydro-5H-cyclopenta[b]pyridinyl, 6,7-dihydro-5H-cyclopental[c]pyrindinyl, 6,7-dihydro-5H-cyclopenta[d]pyrimidinyl, 5,6,7,8-tetrahydro-5,8-ethanoquinazolin-4-yl, and 6,7,8,9-tetrahydropyrimido[4,5-b]indolizin-4-yl, and the N-oxide thereof and a protected derivative thereof.

“Heteroarylalkyl” means an alkyl group, as defined herein, substituted with at least one, specifically one or two heteroaryl group(s), as defined herein.

“Heterocycloalkyl” means a saturated or partially unsaturated (but not aromatic) monovalent monocyclic group of 3 to 8 ring atoms or a saturated or partially unsaturated (but not aromatic) monovalent fused or spirocyclic bicyclic group of 5 to 12 ring atoms in which one or more, specifically one, two, three, or four ring heteroatoms where each heteroatom is independently O, S(O)ₙ (n is 0, 1, or 2), —NH—, or —N═, the remaining ring atoms being carbon. One or two ring carbon atoms may be replaced by a —C(O)—, —C(S)—, or —C(═NH)- group. Fused bicyclic radical includes bridged ring systems. Unless otherwise stated, the valency of the group may be located on any atom of any ring within the radical, valency rules permitting. When the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, pethydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydiocyclopetna[c]pyrrolyl. octahydroindolyl, octahydroisoindolyl, decahydroisoquinolyl, 2,6-diazaspirol[3,3]heptan-2-yl, tetrahydrofuryl, and tetrahydropyranyl, and the derivatives thereof and N-oxide or a protected derivative thereof.

“Heterocycloalkylalkyl” means an alkyl radical, as defined herein, substituted with one or two heterocycloalkyl groups, as defined herein, e.g.. morpholinylmethyl, N-pyrrolidinylethyl, and 3-(N-azetidinyl)propyl, and the like.

“Hydroxyalkyl” means an alkyl group, as defined herein, substituted with at least one, particularly. 1, 2, 3, or 4, hydroxy groups.

“Phenylalkyl” means an alkyl group, as defined herein, substituted with one or two phenyl groups.

“Optional” or “optionally” means thai the subsequently described evenror circumstance may or may not occur, and that the description includes instances where said event or circumstance occurs and instances in which it decs not. One of ordinary skill in the art would understand that with respect to any molecule described as containing one or more optional suhstituents, only sterically practical and/or synthetically feasible compounds are meant to be included. “Optionally substituted” refers to all subsequent modifiers in a term, unless stated otherwise. A list of exemplary optional substitutions is presented below in the definition of “substituted.”

“Optionally substituted aryl” means an aryl group, as defined herein, optionally substituted with one, two, three, or four suhstituents where the substituents are independently acyl, acylamino, acyloxy, alkyl, haloalkyl, hydroxyalkyl, alkoxyalkyl, aminoalkyl, alkylaminoalkyl, dialkylaminoalkyl, alkenyl, alkoxy, alkenyloxy, halo, hydroxy, alkoxycarhonyl, alkenyloxycarhonyl, amino, alkylamino, dialkylamino, nitro, aminocarbonyl, alkylaminocarbonyl, dialkylaminocarbonyl, carhoxy, cyano, alkylthio, alkylsulfinyl, alkylsulfonyl, aminosulfonyl, alkylaminosulfonyl, dialkylaminosulfonyl, alkylsulfonylamino, or aminoalkoxy; or aryl is pentafluorophenyl. Within the optional substituents on “aryl”, the alkyl and alkenyl, either alone or as part of another group (including, for example, the alkyl in alkoxycarbonyl), are independently optionally substituted with one, two, three, four, or five halo (e.g. alkoxycarbonyl includes trifluoromethlyoxycarbonyl).

“Optionally substituted arylalkyl” means an alkyl group, as defined herein, substituted with optionally substituted aryl, as defined herein.

“Optionally substituted cycloalkyl” means a cyctoalkyl group, is defined herein, substituted with one, two, or three groups where the groups are independently acyl, acyloxy, acylamino, alkyl, haloalkyl, hydroxyalkyl, alkoxyalkyl, aminoalkyl, alkylaminoalkyl, dialkylaminoalkyl, alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, or cyano. Within the above optional substituents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl,

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, three, or four substituents where the substituents are independently acyl, acylamino, acyloxy, alkyl, haloalkyl, hydroxyalkyl, alkoxyalkyl, aminoalkyl, alkylaminoalkyl, dialkylaminoalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarhonyl, carboxy, cyano, alkylthio, alkylsulfinyl, alkylsulfonyl, ammosulfonyl, alkylaminosulfonyl, dialkylaminosulfonyl, alkylsulfonylamino, aminoalkoxy, alkylaminoalkoxy, or dialkylaminoalkoxy. Within the optional substiluents on “heteroaryl”, the alkyl and alkenyl, either alone or as part of another group (including, for example, the alkyl in alkoxycarbonyl). are independently optionally substituted with one, two, three, four, or five halo (e.g. alkoxycarbonyl includes trifluoromethyloxycarbonyl).

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, three, or four substituents where the substituents are independently acyl, acylamino, acyloxy, alkyl, haloalkyl, hydroxyalkyl, alkoxyalkyl, aminoalkyl, alkylaminoalkyl, dialkylaminoalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarhonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or phenylalkyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo (e.g. alkoxycarbonyl includes trifluromethyloxycarbonyl).

“Optionally substituted heterocycloalkylalkyl” means an alkyl group, as defined herein, substituted with al least one, specifically one or two, optionally substituted heterocycloalkyl group(s) as defined herein.

“Optionally substituted phenyl” means a phenyl group optionally substituted with one, two, or three substituents where the substituents are independently acyl, acylamino, acyloxy, alkyl, haloalkyl, hydroxyalkyl, alkoxyalkyl, aminoalkyl, alkylaminoalkyl, dialkylaminoalkyl, alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carhoxy, cyano, alkylthio, alkylsulfinyl, alkylsulfonyl, aminosulfonyl, alkylaminosulfonyl, dialkylaminosulfonyl, alkylsulfonylamino, or aminoalkoxy. “Optionally substituted phenyl” in addition includes pentafluorophenyl. Within the optional substituents on “phenyl”, the alkyl and alkenyl, either alone or as part of another group (including, for example, the alkyl in alkoxycarbonyl), are independently optionally substituted with one, two, three, four, or five halo (e.g. alkoxycarbonyl includes trifluoromethyloxycarbonyl).

“Optionally substituted phenylalkyl” means an alkyl group, as defined herein, substituted with one or two optionally substituted phenyl groups, as defined herein.

“Oxo” means tin oxygen which is attached via a double bond.

“Yield” for each of the reactions described herein is expressed as a percentage of the theoretical yield.

“Metabolite” refers to the break-down or end product of a Compound or its salt produced by metabolism or biotransformation in the animal or human body; for example, biotransformation to a more polar molecule such as by oxidation, reduction, or hydrolysis, or to a conjugate (see Goodman and Gilman. “The Pharmacological Basis of Therapeutics” 8.sup.th Ed., Pergamon Press, Gilman et al. (eds). 1990 for a discussion of biotransformation). As used herein, the metabolite of a Compound of the invention or its salt may be the biologically active form of the Compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a Compound of the present invention is known to one of skill in the an in light of the present disclosure.

“Patient” for the purposes of the present invention includes humans and other animals, particularly mammals, and other organisms. Thus the methods are applicable to both human therapy and veterinary applications. In a specific embodiment the patient is a mammal, and in a more specific embodiment the patient is human.

A “pharmaceutically acceptable salt” of a Compound means a salt that is pharmaceutically acceptable and that possesses the desired pharmacological activity of the parent compound. It is understood that the pharmaeeuiically acceptable salts are non-toxic. Additional information on suitable pharmaceutically acceptable salts can be found in Remington's Pharmaceutical Sciences, 17^{th} ed.. Mack Publishing Company, Easton, Pa. 1985, which is incorporated herein by reference or S. M. Berge, et al., “Pharmaceutical Salts,” J. Pharm. Sci., 1977:66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trichloroacetic acid, propionic acid, hexanoic acid, cyclopentanepropinnic acid, glycolic acid, pyruvic acid, lactic acid, oxalic acid, maleic acid, malonic acid, succinic acid, lumaric acid, tartaric acid, citric acid, benzoic acid, cinnamic acid, 3-(4-ydroxybenzoyl)benzoic acid, mandelie acid, methanesulfonic acid, ethanesulfonic acid, 1.2-ethanedisulfonic acid. 2-hydroxyethanesulfonic acid, benzenesulfunic acid, 4-chlorobenzenesulfonic acid, 2-naphthalenesulfonic acid, 4-toluenesulfonic acid, camphorsulfonic acid, glucoheptonic acid, 4.4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent Compound is replaced by a metal ion, such as sodium, potassium, lithium, ammonium, calcium, magnesium, iron, zinc, copper, manganese, aluminum stalts and the like. Specific salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimelhylamine, diethylamide, triethylamine, tripropylamine, ethanolamine, 2-dimethylamimoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and te like. Exemplary organic bases are isopropylamine, diethylamine, ethanolamine, trimethylamine, dicyclohexylamine, choline, and caffeine, “Platin(s),” and “platin-containing agent(s)” include, for example, cisplatin, carboplatin, and oxaliplatin.

“Prodrug” refers to compounds that are transformed (typically rapidly) in vivo to yield the parent Compound of the above Formula e, for example, by hydrolysis in blood. Common examples include, but are not limited to, ester and amide forms of a Compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and and arylalkyl esters such as, hut not limited to benzyl. Examples of pharmaceuttcally acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T, Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pcergamon Press, 1987, both of which are incorporated herein by reference for all purposes.

“Therapeutically effective amount” is an amount of a Compound of the invention, that when administered to a patient, ameliorates a symptom of the disease. The amount of a Compound of the invention which constitutes a “therapeutically effective amount” will vary depending on the compound, the disease state and its severity, the age of the patient to be treated, and the like. The therapeutically effcctive amount can be determined routinely by one of ordinary skill in the art having regard to their knowledge and to this disclosure.

“Preventing” or “prevention” of a disease, disorder, or syndrome includes inhibiting the disease from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

“Treating” or “treatment” of a disease, disorder, or syndrome, as used herein, includes (i) inhibiting the disease, disorder, or syndrome, i.e., arresting its development; and (ii) relieving ihc disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will he ascertainable with routine experimentation by one of ordinary skill in the art.

The compounds disclosed herein also include all pharmaceutically acceptable isotonic variations, in which at least tine atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C: isotopes of nitrogen, such as ¹⁵N; isotopes of oxygen, such as ¹⁷O and ¹⁸O; isotopes of phosphorus, such as ³¹P and ³²P; isotopes of sulfur, such as .sup. ³⁵S; isotopes of fluorine, such as ¹⁸F; and isotopes of chlorine, such as ³⁶CI. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in dntg and/or substrate tissue distribution studies,

### Embodiments of the Invention

The following paragraphs present a number of embodiments of compounds of the invention. In each instance the embodiment includes both the recited compounds, as well as a single stereoisomer or mixture of stereoisomers thereof, as well as a pharmaceutically acceptable salt thereof.

### Embodiments (A.1)

In one embodiment, the Compound of Formula I is that where R^{5a} is hydrogen or alkyl and R^{5c}, R^{5d}, R^{5e}, R^{5f}, and R^{5g} are hydrogen; and all other groups are independently as defined in the Summary of the Invention for a Compound of Formula I. In another embodiment, the Compound of Formula I is that where R^{5a} is alkyl and R^{5e}, R^{5d}, R^{5e}, R^{5f}, and R^{5g} are hydrogen; and all other groups are independently as defined in the Summary of the Invention for a Compound of Formula I.

### Embodiments (A2)

In another embodiment, the Compound of Formula I is that where R^{5h} is (C₁₋₃)alkyl and R^{5a}, R^{5c}, R^{5d}, R^{5e}, R^{5f}, R^{5g}, and R^{5h} are hydrogen; and all other groups are independently as defined in the Summary of the Invention for a Compound of Formula I. In another embodiment, the Compound of Fonmula I is that where R^{5h} is halo(C₁₋₃)alkyl and R^{5a}, R^{5c}, R^{5d}, R^{5e}, R^{5f}, R^{5g}, and R^{5h} are hydrogen: and all oilier groups are independently as defined in the Summary of the Invention for a Compound of Formula I. In another embodiment, the Compound of Formula I is that where R^{5h} is methyl and R^{5a}, R^{5e}, R^{5d}, R^{5e}, R^{5f}, R^{5g}, and R^{5h}, are hydrogen; and all other groups are independently as defined in the Summary of the Invention for a Compound of Formula I. In another embodiment, the Compound of Formula I is that where R^{5h} is methyl; R^{5a}, R^{5c}, R^{5d}, R^{5e}, R^{5f}, R^{5g}, and R^{5h} are hydrogen; and all other groups are independently as defined in ihe Summary of the Invention for a Compound of Formula I.

### Embodiments (A3)

In another embodiment, the Compound of Formula I is that where R^{5c} is hydrogen or alkyl and R^{5a}, R^{5d}, R^{5e}, R^{5f}, and R^{5g} are hydrogen; and all other groups are independently as defined in the Summary of the Invention for a Compound of Formula I. In another embodiment, the Compound of Formula I is that where R^{5c} is alkyl and R^{5a}, R^{5d}, R^{5e}, R^{5f}, are hydrogen; and all other groups are independently as defined in the Summary of the Invention for a Compound of Formula I.

### Embodiments (A4)

In another embodiment, the Compound of Formula I is that where R^{5h} is hydrogen or halo and R^{5a}, R^{5c}, R^{5d}, R^{5e}R^{5f}, R^{5g} are hydrogen; and all other groups are indcpcndenily as defined in the Summary of the Invention for a Compound of Formula I. In another embodiment, the Compound of Formula I is that where R^{5h} is halo and R^{5a}, R^{5c}, R^{5d}, R^{5e}, R^{5f}, R^{5g} are hydrogen; and all other groups are independently as defined in the Summary of the Invention for a Compound of Formula I. In another embodiment, the Compound of Formula I is that where R^{5h} is fluoro and R^{5a}, R^{5c}, R^{5d}, R^{5e}, R^{5f}, R^{5g} are hydrogen; and all other groups are independently as defined in the Summary of the Invention for a Compound of Formula I.

Another embodiment of ihe Invention is directed to a Compound of Formula I(a)

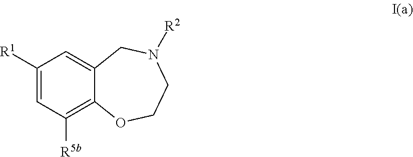

where R¹ and R² are independently as defined in the Summary of the Invention for a Compound of Formula I.

In another embodiment of a compound of Formula Ia, R^{5b} is methyl, ethyl, propyl, or trifluoromcethyl.

In another embodiment of a compound of Formula Ia, R^{5b} is methyl, or trifluoromcethyl.

### Embodiment (1)

In another embodiment, the Compound of Formula 1(a) is that where
R¹ is phenyl opitionally substituted with one, two, three R⁶ groups; or
R¹ is heteroaryl optionally substituted with one, two, or three R⁷;
R² is heteroaryl substiuitccl with R³, R^{3a}, R^{3b}, R^{3c}, and R^{3d};
R³, R^{3a}, R^{3b}, R^{3c}, and R^{3d} are independently hydrogen; cyano; alkyl; alkenyl; halo; haloalkyl; hydroxyalkyl; alkoxyalkyl; cyanoalkyl; SR¹²; —S(O)₂R^{2o}; carboxy; alkoxycarbonyl; halocarbonyl; —NR¹¹R^{11a}; —OR^{11a}; phenyl optionally substituted with one or two groups which are independently alkyl or halo; phcenylalkyl optionally substituted with one or two R¹⁹; cycloalkyl; cycloalkylalkyl; heterocycloalkyl optionally substitiuted with one or two groups which are independently alkyl, alkoxycarbonyl, or benzyloxycarbonyl; heterocycloalkylalkyl optionally substituted with one or two groups which are independently alkyl, alkoxycarbonyl, or benzyloxycarbonyl; heteroaryl; heteroarylalkyl; or alkyl substituted with one or two R¹⁶; or
two of R³, R^{3a},R^{3b}, R^{3c}, and R^{3d}, when attached to the same carbon, form a cycloalkyl or a heterocycoalkyl; and the other of R³, R^{3a}, R^{3b}, R^{3c}, and R^{3d} are hydrogen;
each R⁶, when R⁶ is present, is independently nitro; cyano; halo; alkyl; halo; haloalkyl; —OR^{8a}; —NR⁸R^{8a}; —C(O)NR⁸R^{8a}; —S(O)₂R⁸; —NR⁸C(O)R⁹; —NR⁸S(O)₂R^{8a}; —NHC(O)NHR⁹; carboxy, —C(O)OR⁹; or heteroaryl optionally substituted with 1, 2, or 3 R¹⁴;
each R⁷, when R⁷ is present, is independently oxo; nitro; cyano; alkyl; alkenyl; halo; haloalkyl; hydroxyalkyl; alkoxyalkyl; —OR^{8a}; —SR¹³; —S(O)R¹³; —S(O)₂R^{13a}; -—NR⁸R^{8a}; —C(P)NR⁸R^{8a}; —NR⁸C(O)OR⁹; —NR⁸C(O)R⁹; —NR⁸S(O)₂R^{8a}; —NR⁸C(O)NR^{8a}R⁹; —C(O)OR⁹; halocarbonyl; —S(O)₂NR⁸R⁹; alkylsulfonylalkyl; alkyl substituted with one or two —NR⁸R^{8a}; alkyl substituted with one or iwo —NR⁸C(O)R^{8a}; alkyl subsiitutcd with one or two —NR⁸C(O)OR⁹; alkyl substituted with one or iwo —S(O)₂R^{13a}; cycloalkyl; cycloalkylalkyl; heterocycloalkyl optionally substituted with one or two groups which are independently alkyl or amino; phenyl; phenylalkyl; heterocycloalkylalkyl; heteroaryl; or heteroarylalkyl;
R⁸, R¹¹, R¹⁵, R¹⁷, and R¹⁸ are independently hydrogen, alkyl, alkenyl, alkynyl, hydroxyalkyl, alkoxyalkyl, or haloalkyl;
R^{8a}; R^{11a}; and R^{15a} are independently hydrogen; alkyl; alkenyl; alkynyl; haloalkyl; hydroxyalkyl; cyanoalkyl; aminoalkyl; alkylaminoalkyl; dialkylaminoalkyl; alkoxyalkyl; carboxyalkyl; cycloalkyl; cycloalkylalkyl; heterocycloalkyl optionally substituted with one or two groups which are independently alkyl, alkoxycarbonyl, or benzyloxy; heterocycloalkylalkyl oplionally substituted with one or two groups which are independently alkyl, alkoxycarbonyl, or benzyloxy; phenyl oplionally substituted with one or two groups which are independently halo, alkyl, or alkoxy; phenylalkyl; heteroaryl; or heteroarylalkyl;
R⁹ is hydrogen; alkyl; alkenyl; alkynyl; hydroxyalkyl; alkoxyalkyl; aminoalkyl; alkylaminoalkyl; dialkylaminoalkyl; haloalkyl; hydroxyalkyl substiuited with one, two, or three groups which are independently halo, amino, alkylamino, or dialkylamino; alkyl substituted with one or two aminocarbonyl; phenyl; phenylalkyl; cycloalkyl; cycloalkylalkyl optionally substituted with one or two groups which are independently amino or alkyl; heterocycloalkyl optionally substituted with one or two groups which are independently alkyl, alkoxycarbonyl, or henzyloxy; or heterocyeloalkylalkyl optionally substituted with one or two groups which are independently alkyl, alkoxycarbonyl, or benzyloxy;
R¹² is alkyl or phenylalkyl;
R¹³ is alkyl, hydroxyalkyl, or haloalkyl; and
R^{13a} is hydroxy, alkyl, haloalkyl, hydroxyalkyl, or heterocycloalkyl optionally substituted with one or two groups which are independently halo, amino, alkylamino, dialkylamino, hydroxy, alkyl, or hydroxyalkyl;
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarlionyl, alkylaminocarbonyl, dialkylaminocarbonyl, or phenyl;
each R¹⁶ is independently halo, —NR¹¹R^{11a}, —NR¹⁵S(O)R^{15a}; —OC(O)R¹⁷, or —OR¹⁸;
each R¹⁹ is independently halo, alkyl, haloalkyl, amino, alkylamino, dialkylamino, or alkoxy; and
R²⁰ is amino, alkylamino, dialkylamino, or heterocycloalkyi.

### Embodiment (B)

In another embodiment, the Compound of Formula I(a) is that where R¹ is heteroaryl optionally substituted with one, two, or three R⁷ groups; where each R⁷ independently of each other (when R⁷ is present) and all other groups arc independently as defined in the Summary of the Invention for a Compound of Formula I or as defined in any of Embodiments (A1), (A2), (A3), (A4), and (1). In another embodiment, the Compound is according to Formula 1(a) where R¹ is 3.4-dihydro-2H-pyrido[3.2-][1.4 ]oxazinyl, pyrido[2.3-H]pyrazinyl, imidazo[2-a[pyrimidinyl, imidazo[1,2-a]pyridinyl, triazolo[1.5- a]lpyridinyl, inidolyl, 2,3-dihydrobenznfuranyl, benzo[b]thienyl, quinolinyl, benzimidazolyl, indazolyl, 1H-pyrrolo[2,3-b]pyridinyl, pyridinyl, pyrimidinyl, pyridazinyl, thienyl, thiazolyl, benzothiazolyl, imidazopyridinyl, pyrazolopyridinyl, pyrrolopyridinyl, or thiazolopyridinyl, where R¹ is optionally substituted with one, two, or three R⁷; where each R⁷ independently of each other (when R⁷ is present) and all other groups are independently as defined in the Summary of the Invention for a Compound of Formula I or as defined in any of Embodiments (A1), (A2), (A3), (A4), and (1).

### Embodiments (H1)

In another embodiment, the Compound is according to Formula I(a) where R¹ is a 9-membered heteroaryl optionally substituted with one, two, or three R⁷; where each R⁷ independently of each other (when R⁷ is present) and all other groups are independently as defined in the Summary of the Invention for a Compound of Formula I or as defined in any of Embodiments (A1), (A2), (A3), ( A4). and (1). In another embodiment, the Compound is according to Formula I(a) where R¹ is benzimidazolyl, imidazo[4,5- b]pyridinyl, imidazo[4.5-c]pyridinyl, 3H-imidazo[4.5-c]pyridinyl, indazolyl, 1H- pyrazolo[3,4-b]pyridinyl, indolyl, 1H-pyrrolo[2.3-b]pyridinyl, 1H-pyrrolo[3.2-b]pyridinyl, benzo[d]thiazolyl, thiazolo[4.5-b]pyridinyl, thiazolo[4.5-c]pyridinyl, thiazolo[5.4-c]pyridinyl, or thiazolo[5.4-b]pyridinyl, and R¹ is optionally substituted with one, two, or three R⁷; where each R⁷ independently of each other (when R⁷ is present) and all other groups are independently as defined in the Summary of the Invention for a Compound of Formula I or as defined in any of Embodiments (A1), (A2), (A3), (A4), and (1).

### Embodiments (B1)

In another embodiment, the Compound, is according to Formula I(a) where R¹ is 3H-imidazo[4.5-b]pyridinyl, 1H-imidazo[4,5-b]pyridinyl, 3H- imidazo[4.5-c]pyridinyl, or 1H-imidazo[4.5-c]pyridinyl, where R¹ is optionally substituted with one, two, or three R⁷ groups; where each R independently of each other (when R⁷ is present) and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3H- imidazo[4,5-b]pyridin-5-yl, 1H-imidazo[4,5-b]pyridin-5-yl, 3H-imidazo[4,5-b]pyridin-6-yl, 1H-imidazo[4,5-b]pyridin-6-yl, 3H-imidazo[4,5-c]pyridin-6-yl, 1H-imidazo[4,5-c]pyridin-6- yl, 3H-imidazo[4,5-c]pyridin-5-yl, or 1H-imidazo[4,5-c]pyridin-5-yl, where R¹ is optionally substituted with one, two, or three R⁷ groups; where each R⁷ independently of each other (when R⁷ is present) and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3H-imidazo[4,5-b]pyridin-5-yl, 1-imidazo[4,5-b]pyridin-yl, 3H-imidazo[4.5- b]pyridin-6-yl, 1H-imidazo[4,5 -b]pyridin-6-yl, 3H-imidazo[4,5-c]pyridin-6-yl, 1H- imidazo[4,5-c]pyridin-6-yl, 3H-imidazo[4,5-c]pyridirin-5-yl, or 1H-imidazo[4,5-c]pyridin-5- yl, where R¹ is optionally substituted one or two R⁷; each R⁷, when R⁷ is present, is independently halo, alkyl, cycloalkyl, haloalkyl, hydroxyalkyl, alkoxyalkyl, alkyl substituted with one or two —NR⁸R^{8a}, alkyl substituted with one or two —NR⁸C(O)OR⁹, —NR⁸R^{8a}, or —NR⁸C(O)OR⁹; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3H-imidazo[4,5-b]pyridin-5-yl, 1H-imidazo[4,5-b]pyridin-5-yl, 3H-imidazo[4.5- b]pyridin-6-yl, 1H-imidaxo[4.5-b]pyridin-6-yl, 3H-imidazo[4,5-c]pyridin-6-yl, 1H- imidazo[4,5-c-]pyridin-6-yl, 3H-imidazo][4,5-c]pyridin-5-yl, or 1H-imidazol[4.5-c]pyridin-5- yl, where R¹ is optionally substituted with one or two R⁷; each, R⁷, when R⁷ is present, is independently halo, alkyl, cycloalkyl, haloalkyl, hydroxyalkyl, alkoxyalkyl, alkyl substituted with one or two —NR⁸R^{8a}, alkyl substituted with one or two —NR⁸C(O)OR⁹, —NR⁸R^{8a}, or —NR⁸C())OR⁹; R⁸ and R^{8a} are independently hydrogen or alkyl; R⁹ is alkyl, benzyl, or haloalkyl; and all other groups are independently as defined in the Summary of the Invention for a Compound of Formula I or as defined in any of Embodiments (A1), (A2), (A3), (A4), and (1).

### Embodiments (B2)

In another embodiment, the Compound is according to Formula I(b1)or I(b2)

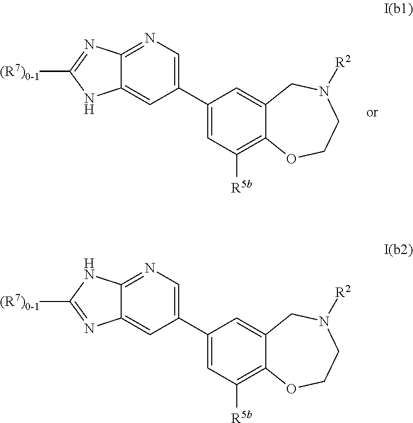

where R⁷, when R⁷ is present, is halo, alkyl, cycloalkyl, haloalkyl, hydroxyalkyl, alkoxyalkyl, alkyl substituted with one or two —NR⁸R^{8a}, alkyl substituted with one or two —NR⁸C(O)OR⁹, —NR⁸R^{8a}, or —NR⁸C(O)OR⁹; and R² and all other groups are independently as defined in the Summary of the Invention for a Compound of Formula I or as defined in any of Embodiments (A1), (A2), (A3), (A4), and (1). In another embodiment, the Compound is according to Formula I(b1) or I(b2), where R⁷, when R⁷ is present, is alkyl, cycloalkyl, haloalkyl, hydroxyalkyl, alkyl substituted with one or two —NR⁸C(O)OR⁹, —NR⁸RS^{8a}, or —NR⁸C(O)OR⁹; R⁸ is hydrogen or alkyl; R^{8a} is hydrogen, alkyl, or haloalkyl; R⁹ is alkyl or benzyl; and R² and all other groups are independently as defined in the Summary of the Invention for a Compound of Formula I or as defined in any of Embodiments (A1), (A2), (A3), (A4), and (1). In another embodiment, the Compound is according to Formula I(b1) or I(b2), where R⁷, when R⁷ is present, is methyl, ethyl, n-propyl, isopropyl, cyclopropyl, cyclobulyl, monofluoromethyl, difluorpmethyl, trifluoroniethyl. 1-hydroxyelhyl, 2-hydroxyethyl, amino, methylamino, ethylamino, methoxycarbonylamino, benzyloxycarbonylamino, aminomethyl, methylaminomethyl, or dimethylaminomethyl; and R² and all other groups are independently as defined in the Summary of the Invention for a Compound of Formula I or as defined in any of Embodiments (A1), (A2). (A3), (A4), and (1).

### Embodiments (B3)

In another embodiment, the Compound of Formula I is according to Formula I(a) where R¹ is benzo[d]thiazolyl, thiazolo[5.4-b]pyridinyl, thiazolo[5.4-c]pyridinyl, thiazolo[4,5-b]pyridinyl, or thiazalo[4,5-c]pyridinyl, where R¹ is optionally substituted with one, two, or three R⁷ groups: where all other groups and each R⁷, when R⁷ is present, are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is benzo[d]thiazol-5-yl, benzo[d]thiazol-6-yl, thiazolo[5,4-b[pyridin-5-yl, thiazolo[5,4- b]pyridin-6-yl, thiazolo[5,4-c]pyridin-6-yl, thiazolo[4,5-b]pyridin-5-yl, thiazolo[4,5- b]pyridin-6-yl, or thiazolo[4.5-c]pyridin-6-yl, where R¹is optionally substituted with one, two, or three R⁷ groups: where all other groups and each R⁷, when R⁷ is present, are independently as defined in the Summary of the Invention for a Compound of Formula I or as defined in any of Embodiments (A 1), (A2), (A3), (A4), and (1). In another embodiment, the Compound of Formula I is according to Formula I(a) where R¹ is thiazolo[5,4-b]pyridin-6-yl or thiazolo[4.5-b]pyridin-6-yl optionally subsiituled with one R⁷ where R⁷ is alkyl, —NR⁸R^{8a}, or —NR⁸C(O)OR⁹; and other groups are independently as defined in the Summary of the Invention for a Compound of Formula I or as defined in any of Embodiments (A 1), (A2), (A3), (A4). and (1). In another embodiment, the Compound of Formula I is according to Formula I(a) where R¹ is thiazo[5,4-b]pyridin-6-yl or thiazolo[4.5-b]pyridin-6-yl optionally substituted with one R⁷ where R⁷ is —NR⁸R^{8a}; and other groups are independently as defined in the Summary of the Invention for a Compound of Formula I or as defined in any of Embodiments (A1), (A2), (A3), (A4), and (1), in another embodiment, the Compound of Formula I is according to Formula I(a) where R¹ is thiazoio[5.4-b]pyridin-6-yl or thiaxolo[4,5-b]pyridiun-6-yl optionally substituted with one R⁷ where R⁷ is allkyl, —NR⁸R^{8a}, or —NR⁸C(O)OR⁹; each R⁸, R^{8a}, and R⁹, independently of each other, are hydrogen or alkyl; and other groups arc independently as defined in the Summary of the Invention for a Compound of Formula I or as defined in any of Embodiments (A1), (A2), (A3), (A4), and (1).

### Embodiments (B4)

In another embodiment, the Compound is according to Formula I(c1)or I(c2)

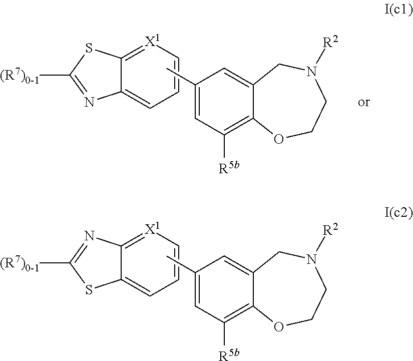

where X¹ is N or CH; R⁷ (when present), R², and all other groups are independently as defined in the Summary of the Invention for a Compound of Formula of as defined in any of Embodiments (A1), (A2), (A3), (A4), and (1). In another embodiment, the Compound is according to Formula I(c1) or i(c2) where X¹ is N or CH; R⁷, when R⁷ is present, is alkyl, —NR⁸R^{8a}, or —NR⁸C(O)R⁹; and R² and all other groups are independently as defined in the Summary of the Invention for a Compound of Formula I or as defined in any of Embodiments (A 1), (A2), (A3), (A4), and (1). In another embodiment, the Compound is according to; Formula I(c1) or I(c2) where X¹ is N or CH; R⁷, when R⁷ is present, is alkyl, —NR⁸R^{8a}, or —NR⁸C(O)R⁹; each R⁸ and R^{8a} are independently hydrogen or alkyl and R⁹ is alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is N or CH; R⁷, when R⁷ is present, is C₁₋₃alkyl, amino, or C₁₋₃-alkylcarbonylamino; and R² and all other groups are independently as defined in the Summary of the Invention for a Compound of Formula I or as defined in any of Embodiments (A1), (A2), (A3), (A4), and (1). In another embodiment, the Compound is according to Formula I(c1) or I(c2) where X¹ is N orCH; R⁷, when R⁷ is present, is —NR⁸R^{8a} where R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(c1) or I(c2) where X¹ is N or CH; R⁷, when R⁷ is present, is —NR⁸R^{8a} where R⁸ and R^{8a} are independentiy hydrogen or C₁₋₃-alkyl; and R² and all other groups are independently as defined in the Summary of the Invention for a Compound of Formula I or as defined in any of Embodiments (A1), (A2), (A3), (A4), and (1).

### Embodiments (B4a)

In another embodiment, the Compound of Formula I is according to Formula I(c1) or I(c2) where X¹ is N; R⁷ (when present), R² and all other groups are independently as defined in the Summary of the Invention for a Compound of Formula I or as defined in any of Embodiments (A1),(A2), (A3), (A4), and (1). In another embodiment, the Compound of Formula I is according to Formula I(c) where X¹ is N; R⁷, when R⁷ is present, is alkyl, —NR⁸R^{8a}, or —NR⁸C(O)R⁹; and R² and all other groups are independently as defined in die Summary of the Invention for a Compound of Formula I or as defined in any of Embodiments (A 1), (A2), (A3), (A4), and (1). In another embodiment, the Compound of Formula I is according to Formula I(c1)or I(c2) where X¹ is N; R⁷, when R⁷ is present, is alkyl, —NR⁸R^{8a}, or —NR⁸C(O)R⁹; each-R⁸ and R^{8a} are independently hydrogen or alkyl and R⁹ is alkyl; and R² and all other groups are independently as defined in the Summary of the Invention for a Compound of Formula I or as defined in any of Embodiments (A 1), (A2), (A3), (A4), and (1). In another embodiment, the Compound of Formula I is according to Formula I(c1) or I(c2) where X¹ is N; R⁷, when R⁷ is present, is C₁₋₃alkyl, amino, or C₁₋₃- alkylcarbonylamino; and R² and all other groups are independently as defined in the Summary of the Invention for a Compound of Formula I, or as defined in any of Embodiments (A 1), (A2), (A3), (A4), and (1). In another embodiment, the Compound of Formula I is according to Formula I(c1) or I(c2) where X¹ is N; R⁷, when R⁷ is present is —NR⁸R^{8a}; each R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1 ). In another embodiment, the Compound of Formula I is according to Formula I(c1) or I(c2) where X¹ is N:; R⁷, when R⁷ is present, is —NR⁸R^{8a}; each R⁸ and RS^{8a} are independently hydrogen or C₁₋₃-alkyl: and R² and all other groups are independently as defined in the Summary of the Invention for a Compound of Formula I or as defined in any of Embodiments (A1), (A2), (A3), (A4), and (1).

### Embodiments (B4b)

In another embodiment, the Compound of Formula I is according to Formula I(c1) or I(c2) where X¹ is C : R⁷ (when present), R², and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C; R⁷, when R⁷ is present, is alkyl, —NR⁸R^{8a} or —NR⁸C(O)R⁹; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C: R⁷, when R⁷ is present, is alkyl, —NR⁸R^{8a}, or —NR⁸C(O)R⁹; each R⁸ and R^{8a} are independently hydrogen or alkyl and R⁹ is alkyll and R² and all other groups are independently as defined in the Summary of the Invention for a Compound of Formula for as defined in any of Embodiments (A1), (A2), (A3), (A4), and (1). In another embodiment, the Compound of Formula I is according to Formula I(c1) or I(c2) where X¹ is C; R⁷, when R⁷ is present, is C₁₋₃- alkyl, amino, or C₁₋₃-alkylcarbonylamino; and R¹ and all other groups are independently as defined in the Summary of the Invention for a Compound of Formula I or as defined in any of Embodiments (A1), (A2), (A3), (A4), and (1). In another embodiment, the Compound of Formula I is according;lb Formula I(c1) or I(C2) where X¹ is C; R⁷, when R⁷ is present is -—NR⁸R^{8a}; each R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(c1) or I(c2) where X¹ is C: R⁷, when R⁷ is present, is —NR⁸R^{8a}; each R⁸ and R^{8a} are independently hydrogen or C₁₋₃-alkyl; and R² and all other groups are independently as defined in the Summary of the Invention for a Compound of Formula I or as defined in any of Embodiments (A1), (A2), (A3), (A4), and (1).

### Embodiments (B5)

In another embodiment, the Compound of Formula I is according to Formula I(a) where R¹ is benzimidazolyl optionally substituted with one, two, or three R⁷ groups; where all other groups and each R⁷ independently of each other (when R⁷ is present) are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is benzimidazolyl optionally substituted with one or two R⁷ groups; and all other groups and each R⁷(when R⁷ is present) are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is benzimidazolyl optionally substituted with one R⁷; and all other groups are independently as defined in the Summary of the Invention for a Compound of Formula I or as defined in any of Embodiments (A1), (A2), (A3), (A4), and (1).

### Embodiments (B6)

In another embodiment, the Compound of Formula I is according to Formula I(d1) or I(d2)

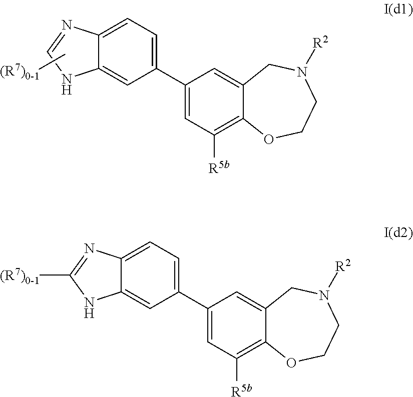

where R⁷, when R⁷ is present, is alkyl, haloalkyl, alkoxyalkyl, —SR¹³, —NR⁸R^{8a}; —NR⁸C(O)R⁹, —NR⁸C(O)OR⁹, —NR⁸C(O)NR^{8a}R⁹, cycloalkyl, heterocycloalkyl, or heteroaryl, and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ , when R⁷ is present, is alkyl , alkoxyalkyl, —SR¹³, —NR⁸R^{8a}, —NR⁸C(O)R⁹, —NR⁸C(O)OR⁹, cycloalkyl, heterocyclalkyl, or heteroaryl; R⁸ and R^{8a} are independently hydrogen or alkyl; R⁹ is alkyl, alkoxyalkyl, or optionally substituted heterocycloalkylalkyl; RK¹³ is alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷, when R⁷ is present, is alkyl, alkoxyalkyl, —SR¹³, —NR⁸R^{8a}, —NR⁸C(O)R⁹, —NR⁸C(O)OR⁹, cycloalkyl, heterocycloalkyl, of heteroaryl; R⁸ and R^{8a} are independently hydrogen or alkyl; R⁹ is alkyl; R¹³ is alkyl; and R² and all other groups are independently as defined in the Summary of the Invention for a Compound of Formula I or as defined in any of Embodiments (A 1), (A2), (A3), (A4), and (1). In another embodiment, the Compound is according to Formula I(d1) or I(d2) where R⁷, when R⁷ is present, is C₁₋₃-alkyl, alkoxyalkyl, —SR¹³, —NR⁸R^{8a}, —NR⁸C(O)R⁹, —NR⁸C(O)OR⁹ , cycloalkyl, heterocycloalkyl, or heteroaryl; R⁸ and R^{8a} are independently hydrogen or C₁₋₃-alkyl; R⁹ is C₁₋₃-alkyl; R¹³ is C₁₋₃-alkyl: and R² and all other groups are independently as defined in the Summary of the Invention for a Compound of Formula I or as defined in any of Embodiments (A1), (A2), (A3), (A4), and (1). In another embodimeht, the Compound is according to Formula I(d1) or I(d2) where R⁷, when R⁷ is present, is methyl, ethyl, n-propyl, isopropyl, methoxymethyl, amino, methylamino, ethylamino, isopropylamino, dimethylamino, 3-piperidinylpropylcarbonylamino, methoxycarbonylamino, 2-(methoxy)-ethyloxycarbonylamino, cyclopropyl, cyclobutyl, cyclopentyl, cyclohexyl, azetidinyl, piperidinyl, or pyridinyl; and R² and all other groups are independently as defined in the Summary of the Invention for a Compound of Formula I or as defined in any of Embodiments (A1), (A2), (A3), (A4), and (1).

### Embodiment (B7)

In another embodiment, the Compound is according to Formula I(d1) or I(d2) where R⁷ is present and is alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is C₁₋₃-alkyl; and R² and all other groups arc independently as defined in the Summary of the Invention for a Compound of Formula I or as defined in any of Embodiments (A1), (A2), (A3), (A4), and (1). In another embodiment, the Compound is according to Formula I(d1) or I((I2) where R⁷is present, and is —NR⁸R^{8a}; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸R^{8a}; R⁸ and R^{8a} are independently hydrogen or alkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is preseni and is —NR⁸R^{8a}; R⁸ and R^{8a} are independently hydrogen or C₁₋₃-alkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸C(O)OR⁹; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is preseni and is —NR⁸C(O)OR⁹; R⁸ and R⁹ are independently hydrogen or alkyl; and all other-groups are independently as defined, in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is —NR⁸C(O)OR⁹; R⁸ and R⁹ are independently hydrogen or C₁₋₃-alkyl; and all other groups are independently as defined in the Summary of the Invention for a Compound of Formula I or as defined in any of Embodimenis (A1), (A2), (A3), (A4), and (1). In another embodiment, the Compound is according to Formula I(d1) or I(d2) where R⁷ is present and is —SR¹³; and all other groups are independently as defined in the Summary of the Invention for a Compound of Formula I or as defined in any of Embodiments (A1), (A2), (A3), (A4),and (1).

In another embodiment, the Compound is according to Formula I(d1) or I(d2) where R⁷ is present and is haloalkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is cycloalkyl; and all other groups are independently as defined in the Summary of the Invention for a Compound of Formula I or as defined in any of Embodiments (A1), (A2), (A3), (A4), and (1). In another embodiment, the Compound is according to Formula I(d1) or I(d2) where R⁷ is present and is cyclopropyl; and all other groups are independently as defined in the Summary of the Invention for a Compound of Formula I or as defined in any of Embodiments (A1), (A2). (A3), (A4), and (1).

### Embodiment (B8)

In another embodiment, the Compound is according to Formula I(f).

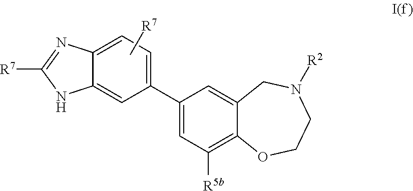

where the R⁷ at the 2-position is —NR⁸R^{8a}; or —NR⁸C(O)OR⁹ and the other R7 is halo; and R²and all other groups are independently as defined in the Summary of the Invention for a Compound of Formula I or as defined in any of Embodiments (A1), (A2), (A3), (A4), and (1). In another embodiment, the Compound of Formula I is according to Formula I(f) where the R⁷at the 2-position is —N⁸R^{8a} or —NR⁸C(O)OR⁹ and the other R⁷ is halo; R⁸, R^{8a}, and R⁹ are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound of Formula I is according to Formula I(f) where the R⁷ at the 2-position is —NR⁸R^{8a} or —NR⁸C(O)OR⁹ and the other R⁷ is halo: R⁸, R^{8a}, and R⁹ are independenily hydrogen or C₁₋₃-alkyl; and R² and all oilier groups are independenily as defined in the Summary of the Invention for a Compound of Formula I or as defined in any of Embodiments (A1), (A2), (A3), (A4), and (1). In another embodiment, the Compound is according to Formula I(f) where the R⁷ at the 2-position is methoxycarbonylamino or amino and the other the R⁷ is fluoro; and R² and all other groups are independently as defined in the Summary of the Invention for a Compound of Formula I or as defined in any of Embodiments (A1), (A2), (A3),(A4), and (1).

### Embodiment (B9)

In another embodiment, the Compound is according to Formula I(a) where R¹ is a 5-membered heteroaryl, where R¹ is optionally substituted with one or two R⁷; each R⁷ (when present), and all other groups are independently as defined in the Summary of the Invention for a Compound of Formula I or as defined in any of Embodiments (A1), (A2), (A3), (A4), and (1).

### Embodiments (B10)

In another emmbodiment, the Compound is according to Formula I(a) where R¹ is thiazol-2--yl, thiazol-4-yl, or thiazol-5-yl, where R¹ is optionally substituted with one or two R⁷: each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazolo-5-yl, where R¹ is optionally substituted with one R⁷; R⁷, all other groups are independently as defined in the Summary of the Invention for a Compound of Formula I or as defined in any of Embodiments (A1), (A2), (A3), (A4), and (1).

### Embodiments (B11)

In another embodiment, the Compound is according to Formula I(a) where R¹ is thiazol-2-yl, lhiazol-4-yl, or thiazol-5-yl, where R¹ is optionally substituted with one or two R⁷: where each R (when present), where each R⁷ is independently alkyl, —NR⁸C(O)OR⁹, —C(O)NR⁸R^{8a}; or each R⁸ and R^{8a} are independently hydrogen or alkyl and R⁹ is alkyl (in another embodiment each alkyl in R⁸, R^{8a}, and R⁹ are C₁₋₃-alkyl); and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazol-5-yl, where R¹ is optionally substituted with one or two R⁷: where each R⁷ (when present), where each R⁷ is independently alkyl, —NR⁸C(O)OR⁹, —C(O)NR⁸R^{8a}, or —NR⁸R^{8a}; each R⁸ and R^{8a} are independently hydrogen or C₁₋₃alkyl and R⁹ is C₁₋₃-alkyl; and all othergroups are indcpcndenllyas defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4-yl, or thiazol-5-yl, where R¹ is optionally substituted with one or two R⁷: each R⁷, when R⁷ is present, is independently methyl, or amino; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is thiazol-2-yl, thiazol-4- yl, or thiazol-5-yl, where R¹ is substituted with two R⁷; where one R⁷, is alkyl and the other R⁷ —NR⁸R^{8a}; and all other groups are independently as defined in the Summary of the Invention for a Compound of Formula for as defined in any of Embodinienls (A1), (A2), (A3), (A4), and (1).

### Embodiments (B12)

In another embodiment, the Compound is according to Formula I(a) where R¹ is thien-2-yl, thien-3-yl, thien-4-yl, or thien-5-yl, where R¹ is optionally substituted with one or two R⁷ groups; where each R⁷ (when present), and all other groups are independently as defined in the Summary of the invention for a Compound of Formula I for as defined in any of Embodiments (A1 ), (A2), (A3), (A4), and (1), In. another embodiment, the Compound is according to Formula I(a) where R¹ is thien-2-yl, thien-3-yl, thien-4-yl, or thien-5-yl, and all other groups are independently as defined in the Summary of the Invention for a Compound of Formula I or as defined in any of Embodiments (A1), (A2), (A3), (A4), and (1).

### Embodiments (B13)

In another embodiment, the Compound is according to Formula I(a) where R⁹ is pyrazol-1-yl, pyrazol-3-yl, pyrazol-4-yl, or pyrazol-5-yl, where R¹is optionally substituted with one or two R⁷ groups: where each R⁷ (when present), and all other groups are independentlyas defined in the Summary of the Invention for a Compound of Formula I or as defined in any of Embodiments (A1), (A2), (A3), (A4), and (1). In another embodiment, the-Compound is according to Formula I(a)where R¹ is pyrazol-1 -yl, pyrazol-3- yl, pyrazol-4-yl, or pyrazol-5-yl; and all other groups are independently as defined in the Summary of the Invention for a Compound of Formula I or as defined in any of Embodiments (A1), (A2), (A3), (A4), and (1).

### Embodiment (B14)

In another embodiment, the Compound is according to Formula I(a) where R¹ is a 6-membcred heteroaryl, where R¹ is optionally substituted with one or two R⁷ groups; where each R⁷ (when present), and all other groups arc independently as defined in the Summary of the Invention for a Compound of Formula I or as defined in any of Embodiments (A1), (A2), (A3), (A4), and (1).

### Embodiments (B15)

In another embodiment, the Compound is according to Formula I(a) where R¹ is pyrimidin-2-yl, pyrimidin-4-yl, pyrimidin-5-yl, pyrimidin-6-yl, where R¹ is optionally subsiituled with one or two R⁷ groups; where each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyrimidin-2-yl, pyrimidin-4-yl, pyrimidin-5-yl, pyrimidin-6-yl, where R¹ is optionally substituted with one R⁷ where R⁷ is —NR⁸R^{8a}; R⁸ and R^{8a} are independently hydrogen or alkyl: and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyrimidin-2-yl, pyrimidin-4-yl, pyrimidin-5-yl, pyrimidin-6-yl, where R¹ is optionally substituted with one R⁷ where R⁷ is —NR⁸R^{8a}; R⁸ and R^{8a} are independently hydrogen or C₁₋₃alkyl; and all other groups are independently as defined in the Summary of the Invention for a Compound of Formula I or as defined in any of Embodiments (A1), (A2), (A3), (A4), and (1). In another embodiment, the Compound, is according to Formula I(a) where R¹ is R¹ is 2-amino-pyrimidin-5-yl; and all other groups are independently as defined in the Summary of the Invention for a Compound of Formula I or as defined in any of Embodiments (A1), (A2), (A3), (A4), and (1).

### Embodiments (B16)

In another embodiment, the Compound is according to Formula I(a) where R¹ is pyridin-2-yl, pyridin-3-yl, pyridin-4-yl, pyridin-5-yl, or pyridin-6- yl, where R¹ is optionally substituted with one or two R⁷ groups; where, each R⁷ (when present), and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyridinyl where R¹ is optionally substituted with one or two R⁷ where each R⁷ is independently halo, cyano, alkylsulfonyalkyl, —OR^{8a}, —C(O)NR⁸RS^{8a}, S(O)₂OH, —S(O)R¹³, —S(O)₂R^{13a}, —S(O)₂NR⁸⁸R⁹, —NR⁸R^{8a}, —NR⁸C(O )OR⁹ , —NR⁸C(O)R⁹, —NR⁸S(O)₂R^{8a}, or heterocycloalkyl optionally substituted with one amino; and all other groups are independently as defined in the Summary of the Invention for a Compound of Formula I or as defined in any of Embodiments (A1), (A2), (A3), (A4), and (1).

### Embodiments (B16a)

In another embodiment, the Compound is according to Formula I(a) where R¹ is pyridinyl where R¹ is optionally substituted with one or two R⁷ where each R⁷ is independently halo, cyano, alkylsulfonylalkyl, —OR^{8a}, —C(O)NR⁸R^{8a}, S(O)₂OH, —S(O)R¹³, —S(O)₂R^{13a}, —S(O)₂NR⁸R⁹, —NR⁸R^{8a}, —NR⁸C(O)OR⁹, —NR⁸C(O)R⁹, —NR⁸S(O₂R^{8a}, heterocycloalkyl optionally substituted with one amino: where
each R⁸ is independently hydrogen, haloalkyl, or alkyl;
each R^{8a} is independently hydrogen, alkyl, benzyl, or phenyl which phenyl is optionally substituted with one or two groups which are independently halo or alkyl:
each R⁹ is independently hydrogen; alkyl; hydroxyalkyl; alkoxyalkyl; aminoalkyl; alkylaminoalkyl; dialkylaminoalkyl; haloalkyl: hydroxyalkyl subsiituled with one, two, or three halo, heterocycloalkylalkyl oplionally substiiuied with one alkyl; heterocycloalkyl oplionally substituled with one alkyl; cycloalkylalkyl oplionally substituted with one amino; cycloalkyl;
R¹³ is alkyl or hydroxyalkyl;
R^{13a} is alkyl; hydroxyalkyl; heterocycloalkyl oplionally substituted with one or two groups which are independently halo, amino, alkylamino, dialkylamino, hydroxy, alkyl, or hydroxyalkyl;
   and all other groups are independently as defined in the Summary of the Invention for a Compound of Formula I or as defined in any of Embodiments (A1), (A2), (A3), (A4), and (1).

### Embodiments (B16b)

In another embodiment, the Compound of Formula I is according to Formula I(e)

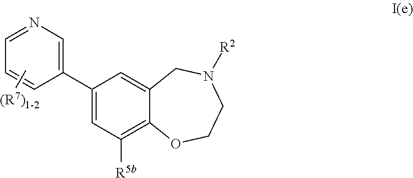

where each R⁷ and R² are indepcedently as defined in the Summary of the Invention for a Compound of Formula I or as defined in any of Embodiments (A1), (A2), (A3), (A4), and (1). in another embodiment, the Compound of Formula I is according to Formula I(e) where each R⁷ is independently as defined in embodiment B16a and R² is as defined in the Summary of the Invention for a Compound of Formula I or as defined in any of Embodiments (A1), (A2), (A3), (A4), and (1).

### Embodiments (B16c)

In another embodiment, the Compound of Formula I is according to Formula I(c1)

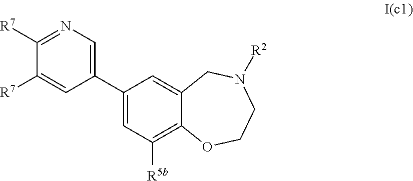

where each R⁷ and R² are independently as defined in the Summary of the Invention for a Compound of Formula I or as defined in any of Embodiments (A1), (A2), (A3), (A4), and (1). In another embodiment, the Compound of Formula I is according to Formula I(e) where each R⁷ is independently as defined in embodiment B16a and R² is as defined in the Summary of the Invention for a Compound of Formula I or as defined in any of Embodiments (A1), (A2), (A3), (A4), and (1). In another embodiment, the Compound of Formula I is according to Formula I(e1) where the R⁷ in the 2-position is hydrogen, halo, cyano, alkoxy, alkyl, or —NR⁸R^{8a} and the R⁷ in the 3-position is —NR⁸S(O)₂R^{8a}; and R² and all other groups are as defined in the Summary of the Invention for a Compound of Formula I or as defined in any of Embodiments (A1), (A2), (A3), (A4), and (1). In another embodiment, the Compound of Formula I is according to Formula I(e1) where the R⁷ in the 2-position is hydroxy or —NR⁸R^{8a} and the R⁷ in the 3-position is —S(O)₂R^{13a}, —S(O)₂R^{13a}, —S(O)₂NR⁸R⁹; and R² and all other groups are as defined in the Summary of the Invention for a Compound of Formuula I or as defined in any of Embodiments (A1), (A2), (A3), (A4), and (1). In another embodiment, the Compound of Formula I is according to Formula I(e1) where the R⁷ in the 2-position is hydroxy or —NR⁸R^{8a}; and the R⁷ in the 3-position is —S(O)R¹³ , —S(O)₂R^{13a}, —S(O)₂NR⁸R⁹; R¹³ is hydroxyalkyl; R^{13a} is alkyl or heterocycloalkyl optionally substituted with one group which is amino, alkyl, hydroxyalkyl, or hydroxy; each R⁸ and R^{8a} are independently hydrogen or alkyl; R⁹ is hydrogen, haloalkyl, alkoxyalkyl, hydroxyalkyl, aminoalkyl, alkylaminoalkyl. dialkylaminoalkyl, cycloalkyl, heterocycloalkyl, heterocycloalkylalkyl, alkyl substituted with one aminocarbonyl, or hydroxyalkyl which is substituted with one amino or 3 halo; and R² and all other groups are as defined in the Summary of the Invention for a Compound of Formula I or as defined in any of Embodiments (A1), (A2), (A3), (A4), and (1).

### Embodiments (B17)

In another embodiment, the Compound of Formula I is according to Formula I(a) where R¹ is pyridazin-3-yl, pyridazin-4-yl, pyridazin-5-yl, or pyriclazin-6-yl, ehere R¹ is optionally substituted with one or two R⁷ groups; where each R⁷ (when present); and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is pyridazin-3-yl, pyridazin-4-yl, pyridazin-5-yl, or pyridazin-6-yl, where R¹ is optionally substituted with one or two R⁷ groups where each R⁷ is independently —NR⁸R^{8a}; R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 3-amino-pyridazin-6-yl; and all other groups are independently as defined in the Summary of the Invention for a Compound of Formula I or as defined in any of Embodiments (A1), (A2), (A3), (A4), and (1).

### Embodiments (B18)

In another embodiment, the Compound is according to Formula I(a) where R¹ is pyrazin-2-yl, pyrazin-3-yl, pyrazin-5-yl, or pyrazin-6-yl, where R¹is optionally substituted with one or two R⁷ groups: where each R⁷ (when present), and all other groups are independently as defined in the Summary of the Invention for a Compound of Formula I or asdefined in any of Embodiments (A1), (A2), (A3), (A4), and (1). In another embodiment, the Compound is according to Formula I(a) where R¹ is pyrazin-2-yl, pyrazin-3- yl, pyrazin-5-yl, or pyrazin-6-yl, where R¹ is optionally substituted with one R⁷ where R⁷ is —NR⁸SR^{8a}; R⁸ and R^{8a} are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 5-amino-pyrazin-2-yl; and R² and all other groups are independently as defined in the Summary of the Invention for a Compound of Formula I or as defined in any of Embodiments (A1), (A2), (A3), (A4), and (1).

### Embodiments (B19)

In another embodiment, the Compound is according to Formula I(a) where R¹ is 1H-pyrrolo[2.3-b]pyridinyl, optionally substituted with one or two R⁷ groups; where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 1H-pyrrolo[2.3-b]pyridin-5-yl, optionally substituted with one or two R⁷ groups; where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 1H-pyrrolo[2,3-b]pyridin-5-yl, optionally substituted with one R7; where the R⁷, when R⁷ is present, and all other groups are independently as defined in the Summary of the Invention for a Compound of Formula I or as defined in anyrof Embodiments (A1), (A2), (A3), (A4), and (1). In another embodiment, the Compound is according to Formula I(a) where R¹ is 1H-pyrrolo[2,3-b]pyridin-5-yl, optionally substituted with one R⁷; R⁷, when R⁷ is present, is methyl of ethyl: and all other groups are independently as defined in the Summary of the Invention for a Compound of Formula I or as defined in any of Embodiments (A1), (A2), (A3), (A4), and (1).

### Embodiments (B20)

In another embodiment, the Compound is according to Formula I(a) where R¹ is indazolyl, optionally substituted with one or two R⁷ groups: where R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indazol-5-yl or indazol-6-yl, where R¹ is optionally substituted with one or two R⁷ groups; where R⁷, when R⁷ is present, and all other groups are indcpcndenily as defined in the Summary of the Invention for a Compound of Formula I or as defined in any of Embodiments (A1), (A2), (A3), (A4), and (1). In another embodiment, the Compound is according to Formula I(a) where R¹ is indazol-5-yl or indazol-6-yl, where R¹ is optionally substituted with one R⁷; R⁷, when present, is alkyl or amino; and R² and all other groups are independently asdefined in the Summary of the Invention for a Compound of Formula I or as defined in any of Embodiments (A1), (A2), (A3), (A4), and (1). In another embodiment, the Compound is according to Formula I(a) where R¹ is indazol-5-yl, indazol-6-yl, or N-methyl- indazol-5-yl; and all other groups are independently as defined in the Summary of the Invention for a Compound of Formula I or as defined in any of Embodiments (A1), (A2), (A3), (A4), and (1).

### Embodiment (B21)

In another embodiment, the Compound is according to Formula I(a) where R¹ is benzimidazolyl substiiuied with two R⁷ groups where each R⁷ is alkyl; and R² and all other groups are independenlly as defined in the Summary of the Invention for a Compound of Formula I or as defined in any of Embodiments (A1), (A2), (A3), (A4), and (1). In another embodiment, the Compound is according to Formula I(a) where R¹ is benzimidazolyl substituted with two R⁷ groups where each R⁷ is C₁₋₃-alkyl; and R² and all other groups are independently as defined in the Summary of the Invention for a Compound of Formula I or as defined in any of Embodiments (A1), (A2), (A3), (A4), and (1).

### Embodiments (B22)

In another embodiment, the Compound is according to Formula I(a) where R¹ is quinolin-2-yl, quinolin-3-yl, quinolin-4-yl, quinolin-5-yl, quinolin- 6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, isoquiinolin-8-yl, quinazolin-2-yl, quinazolin-3-yl, quinazolin-5-yl, quinazolin-6-yl, quinazolin-7-yl, or quinaxolin-8-yl, where R¹ is optionally substituted with one or two R⁷ groups; where each R⁷, when R⁷ is present, and all other groups are independenily as defined in ihe Summary of the Invention for a Compound of Formula I or as defined in any of Embodiments (A1), (A2), (A3), (A4), and (1). In another embodiment, the Compound is according to Formula I(a) where R¹ is quinolin-2-yl, quinolin-3-yl, quinolin-4-yl, quinolin-5-yl, quinolin-6-yl, quinolin-7-yl, quinolin-8-yl, quinazolin-2-yl, quinazolin-3-yl, quinazolin-5-yl, quiazolin-6-yl, quinazolin- 7-yl, or quinazolin-8-yl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is quinolin-3-yl or quiuazolin-6-yl; and R²and all other groups are independently as defined in the Summary of the Invention for a Compound of Formula I or astdefined in any of Embodiments (A1), (A2), (A3), (A4), and (1).

### Embodiments (B24)

In another embodiment, the Compound is according to Formula I(a) where R¹ is 2,3-dihydrohenzofuran-4-yl, 2,3-dihiydrobenzofuran-5-yl, 2,3-dihydrobenzofuran-6-yl, or 2,3-dihydrobcnzofuran-7-yl, where R¹ is optionally substituted with one or two R⁷ groups: where each R⁷ , when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 2,3-dihydrobenzofuran- 4-yl, 2,3-dihydrobenzofuran-5-yl, 2,3-dihydrobenzofuran-6-yl, or 2,3-dihydrobenzofuran-7- yl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is 2,3- dihydrobenzofuran-5-yl; and R² and all other groups are independently as defined in the Summary of the Invention for a Compound of Formula I or as defined in any of Embodiments (A1), (A2), (A3), (A4), and (1).

### Embodiments (25)

In another embodiment, the Compound is according to Formula I(a) where R¹ is indol-1 -yl, indol-2-yl, indol-3-yl, indol-4-yl, indol-5-yl, indol-6-yl, or indol-7-yl, where R¹ is optionally substituted with one or two R⁷ groups: where each R⁷, when R⁷ is present, and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indol-1-yl, indol-2-yl, indol-3-yl, indol-4-yl, indol-5-yl, indol-6-yl, or indpl-7-yl where R¹ is oplionally substituted with one R⁷ where R⁷ is alkyl: and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is indol-5-yl optionally substituted with one R⁷ where R⁷ is alkyl; and all other groups are independently as defined in the Summary of the Invention for a Compound of Formula I or as defined in any of Embodiments (A1), (A2),(A3),(A4), and (1).

### Embodiments (B26)

In another embodiment, the Compound is according to Formula I(a) where R¹ is [2.4]triazolo[5-a]pyridin-2-yl, [1,2,4]triazolo[1,5-a]pyridin-5-yl, [1,2,4]triazplo[1,5-a]pyridin-6-yl, [1,2,4]triazolo[1,5-a]pyridin-7-yl, or [1,2,4]triazolo[1,5- a]pyridin-8-yl, where R¹ is optionally substituted with one or two R⁷ groups; where each R⁷, when R⁷ is present, and all other groups are indcpcriderilly as defined in the Summary of the Invention for a Compound of Formula I or as defined in any of Embodiments (A1), (A2), (A3), (A4), and (1). In another embodiment, the Compound is according to Formula I(a) where R¹ is [1,2,4]triazolo[1,5-a]pyridin-2-yl, [1,2,4]triazolo[1,5-a]pypidin-5-yl, [1,2,4]triazolo[1,5-a]pyridin-6-yl, [1,2,4 [triazolo[1,5-a]pyridin-7-yl, or [1,2,4]triazolo[1.5- a]pyridin-8-yl, where R¹ is optionally substituted with one R⁷ where R⁷ is —NR⁸R^{8a}; R⁸ and R⁸ are independently hydrogen or alkyl; and R² and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is [1,2,4]triazolo[5-a]pyridin-6-yl, or [2.4]triazolo[1,5-a]pyridin-7-yl, optionally substituted with one R⁷ where R⁷ is amino; and all other groups are indepentently as defined in the Summary of the Invention for a Compound of Formula I or as defined in any of Embodiments (A1), (A2), (A3), (A4). and (1).

### Embodiments (B27)

In another embodiment, the Compound is according to Formula I(g)

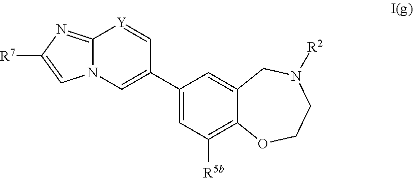

where Y is N or CH; and R² and R⁷ are independently as defined in the Summary of the Invention for a Compound of Formula I or as defined in any of Embodiments (A1), (A2), (A3), (A4), and (1). In another eiubodimeni the Compound of Formula I(g) is that where R7, when present, is —NR⁸R^{8a} or —NR⁸C(O)R⁹; and R² and all oilier groups are independently as defined in the Summary of the Invention for a Compound of Formula I or as defined in any of Embodiments (A1), (A2), (A3), (A4), and (1). In another embodiment the Compound of Formula I(g) is that where R⁷, when present, is —NR⁸R^{8a} or —NR⁸C(O)R⁹; R⁸ and R^{8a} are independently hydrogen or alkyl; R⁹ is alkyl or haloalkyl; and R² and all other groups are independently as defined in the Summary of the Invention for a Compound of Formula I or as defined in any of Embodiments (A1), (A2), (A3), (A4), and (I). In another embodiment the Compound of Formula I(g) is that where R⁷, when present, is —NR⁸R^{8a} or —NR⁸C(O)R⁹; R⁸ and R^{8a} are independently hydrogen or C₁₋₃-alkyl; R⁹ is C₁₋₃-alkyl or halo-C₁₋₃-alkyl: and R²and all other groups are independently as defined in the Summary of the Invention for a Compound of Formula for as defined in any of Embodiments (A1), (A2), (A3), (A4), and (1). In another embodiment the Compound of Formula I(g) is that where R⁷, when present, is amino or trifluoromethylcarbonylamino; and R² and all other groups are independently as defined in the Summary of the Invention for a Compound of Formula I or as defined in any of Embodiments (A1), (A2), (A3), (A4), and (1).

### Embodiments (B28)

In another embodiment, the Compound of Formula I is according to Formula I(a) where R¹ is pyrido[2.3-b]pyrazinyl optionally substituted with one or two R⁷ groups; where R⁷ and all other groups are independently as defined in the Summary of the Invention for a Compound of Formula I for as defined in any of Embodiments (A1), (A2), (A3), (A4), and (1). In another embodiment the Compound of Formula I is according to Formula I(a) where R¹ is unsubstituted pyridol[2.3-b]pyrazinyl where all other groups are independently as defined in the Summary of the Invention for a Compound of Formula I or as defined in any of Embodiments (A1), (A2), (A3), (A4), and (1).

### Embodiments (B29)

In another embodiment the Compound of Formula I is according to Formula I(a) where R¹ is 3,4-dihydro-2H-pyrido[3,2[-b][1,4]oxazinyl optionally substituted with one or two R⁷ groups: where R⁷ and all other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is unsubstituted 3.4-dihydio-2H-pyrido[3.2- b][1, 4]oxazinyl where all other groups are independently as defined in the Summary of the Invention for a Compound of Formula I or as defined in any of Embodiments (A1), (A2), (A3), (A4), and (1).

### Embodiments (C)

In another embodiment, the Compound of Formula I is according to Formula I(a) where R¹ is phenyl optionally substituted with one, two, or three R⁶ groups: where each R⁶, when R⁶ is present, and all other groups are independently as defined in the Summary of the Invention for a Compound of Formula I or as defined in any of Embodiments (A1 ),(A2), (A3), (A4), and (1). In another embodiment, the Compound of Formula I is according to Formula I (a) where R¹ is phenyl optionally substituted with one or two R⁶ groups: where each R⁶, when R⁶ is present, and all other groups are independently as defined in the Summary of the Invention for a Compound of Formula I or as defined in any of Embodiments (A1), (A2), (A3), (A4), and (1).

### Embodiments (C1)

In another embodiment, the Compound of Formula I is according to Formula I(a) where R¹ is phenyl optionally substituted with one, two, or three R⁶ groups; where each R⁶ is independently nitro, halo, alkoxy, —OR⁸a, —S(O)₂R⁸, —NR⁸R^{8a}, —NR⁸S(O)₂R^{8a}, —NR⁸C(O)R⁹, —C(O)NR⁸R^{8a}, —NR⁸C(O)NR^{8a}R⁹, carboxy, alkoxycarbonyl, or heteroaryl optionally substituted with one or two R¹⁴; and all other groups are independently as defined in the Summary of the Invention for a Compound of Formula I or as defined in any of Embodiments (A1), (A2), (A3), (A4), and (1). In another embodiment, the Compound of Formula I is according to Formula I(a) where R¹ is phenyl optionally substituted with one, two, or three R⁶ groups; where each R⁶ is independently —S(O)₂R⁸, —C(O)NR⁸R^{8a} or heteroaryl optionally substituted with one or two R¹⁴; and all other groups are independently as defined in the Summary of the Invention for a Compound of Formula I or as defined in any of Embodiments (A1), (A2), (A3), (A4), and (1).

### Embodiment (C2)

In another embodiment, the Compound is according to Formula I(a) where R¹ is phenyl optionally substituted with one, two, or three R⁶ groups; where each R⁶ is independently nitro, halo, alkoxy, —OR^{8a}, —S(O)₂R⁸, —NR⁸R^{8a}, —N⁸S(O)₂R^{8a}, —NR⁸C(O)R⁹, —C(O)NR⁸R^{8a}; —NR⁸C(O)NR^{8a}R⁹, carboxy, alkoxycarbonyl, or heteroaryl optionally substituted with one or two R¹⁴; each R⁸ is independently hydrogen or alkyl; each R^{8a} is independently hydrogen, alkyl, haloalkyl, optionally substituted cycloalkyl, or optionally substituted heterocycloalkyl; R⁹ is alkyl; R¹⁴, when present is hydroxyalkyl; and all other groups are independently as defined in the Summary of the Invention for a Compound of Formula I or as defined in any of Embodiments (A1), (A2), (A3), (A4), and (1). In another embodiment, the Compound is according to Formula I(a) where R¹ is phenyl optionally substituted with one, two, or three R⁶ groups; where each R⁶ is independently nitro, halo, alkoxy, —OR^{8a}, —S(O)₂R⁸, —NR⁸R^{8a}, —NR⁸S(O)₂R^{8a}, —NR⁸C(O)R⁹, —C(O)NR⁸R^{8a}, —NR⁸C(O)NR^{8a}R⁹, carboxy, alkoxycarbonyl, or heteroaryl optionally substituted with one or two R¹⁴; each R⁸ is independently hydrogen or C₁₋₃-alkyl; each R^{8a} is independently hydrogen, alkyl, haloalkyl, optionally substituted cycloalkyl, or optionally substituted heterocycloalkyl; R⁹ is C₁₋₃-alkyl: R¹⁴, when present is hydroxyalkyl; and all other groups are independently as defined in the Summary of the Invention for a Compound of Formula I or as defined in any of Embodiments (A1), (A2), (A3), (A4), and (1).

### Embodiment (C3)

In another embodiment, the—Compound is according to Formula I(a) where R¹ is phenyl optionally substituted wilh one or two R⁶ groups where each R6 is independently nitro, chloro, methoxy, methylsulfonyl, amino, methylaminocarbonylamino, methylamino, carboxy, methylcarbonylamino, aminocarbonyl, methylaminocarbonyl, ethylaminocarbonyl, n-propylaminocarbonyl, isopropylaminocarbonyl, 2-monofluoroethylaminocarbonyl, 2,2-difluoroethylaminocarbonyl, 2,2,2-trifluoroethylaminocarbonyl, 1,1.1-trifluoroprop-2-ylaminocarbonyl, cyclopropylaminocarbonyl, pyrrolidinylaminocarbonyl, methoxycarbonyl, imidazolyl, imidazolyl substituted with hydroxymethyl, or pyrazolyl; and R² and all other groups are independently as defined in the Summary of the Invention for a Compound of Formula I or as defined in any of Embodiments (A1), (A2), (A3), (A4), and (1).

In a Compound as described by any one of Formula I, I(a), I(b1), I(b2), I(c1), I(c2), I(d1), I(d2), I(e1), I(e2), I(f), and I(g), or by any of the above embodiments (1), (A1), (A2), (A3), (A4), (B), (H1), (H2), (B1), (B2), (B3), (B4), (B4a), (B4b), (B5), (B6), (B8), (B9), (B10),(B11), (B12), (B13), (B14), (B15), (B16), (B16a), (B16b). (B16c), (B17), (B18), (B19), (B20), (B21), (B22), (B23), (B24), (B25), (B26), (B27), (C), (C1), (C2), and (C3), R² can be described according to any of the following embodiments.

### Embodiments (D)

In another embodiment, R² is a 6-membered heteroaryl substituted with R³, R^{3a}, R^{3b}, and R^{3c}, R³, R^{3a}, R^{3b}, and R^{3c} and all other groups are independently as defined in the Summary of the Invention for a Compound of Formula I or as defined in embodiment (1).

### Embodiments (D1)

In another embodiment, R² is pyrimidinyl substituted with R³, R^{3a}, and R^{3b}; where R³, R^{3a}, R^{3b}, and all other groups are indenpendently as defined in the Summary of the Invention for a Compound of Formula I or as defined in embodiment (1).

### Embodiments (D2)

In another embodiment, R² is according to Formula (a)

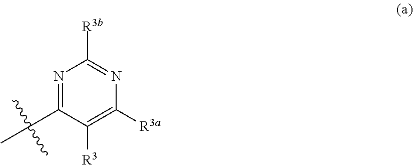

where R³, R^{3a}, and R^{3b} are independenily hydrogen; alkyl; halo; hydroxyalkyl; cyanoalkyl; —NR¹¹R^{11a}; —S(O)₂R²⁰; optionally substituted cycloalkylalkyl; optionally substituted heterocycloalkyl; optionally substituted phenylalkyl; alkyl substituted with one or two R¹⁶; or —OR^{11a}; and all other groups are independently as defined in the Summary of the Invention for a Compound of Formula I or as defined in embodiment (1). In another embodiment R² is according to Formula (a) where R³, R^{3a}, and R^{3b} are independently hydrogen; alkyl; halo: hydroxyalkyl; cyanoalkyl; —NR¹¹R^{11a}; —S(O)₂R²⁰; cycloalkylalkyl; heterocycloalkyl optionally substituted with one or two alkyl; phenylalkyl optionally substituted with one or two R¹⁹; alkyl substituted with one or two R¹⁶; or —OR^{11a}; and all other groups are independently as defined in the Summary of the Invention for a Compound of Formula I or as defined in embodiment (1). In another embodiment, R² is according to Formula (a) where R³, R^{3a},and R^{3b} are independently hydrogen; alkyl; halo; hydroxyalkyl; cyanoalkyl; —NR¹¹R^{11a}; —S(O)₂R²⁰; cycloalkylalkyl; heierocyeloalkyl optionally substituted wiih one or two alkyl; phenylalkyl optionally substituted with one or two R¹⁹; alkyl substituted with one or two R¹⁶; or —OR^{11a}; each R¹⁹ is independently halo, alkyl, haloalkyl, alkoxy, amino, alkylamino, or dialkylamino; each R¹⁶ is independently halo, —NR¹¹R^{11a} or —OC(O)R¹⁷; R¹⁷ is alkyl; each R¹¹ is independently hydrogen, alkyl (in another embodiment each alkyl is C₁₋₃-alkyl), or cycloalkyl; each R^{11a} is independently hydrogen; alkyl (in another embodiment each alkyl is C₁₋₃-alkyl); aminoalkyl; alkylaminoalkyl; dialkylaminoalkyl; phenyl; phenyl substituted with one alkoxy; phenylalkyl; heterocycloalkyl; heterocycloalkyl substituted with one or two alkyl; heterocycloalkylalkyl; heterocycloalkylalkyl substituted vvith one or two alkyl; R²⁰ is amino, alkylamino, dialkylamino, or heterocycloalkyl; and all other groups are independently as defined in the Summary of the Invention for a Compound of Formula I or as defined in embodiment (1). In another embodiment, R² is according to Formula (a) where R³, R^{3a}, and R^{3h} are independently hydrogen:; alkyl (in another embodiment alkyl is C₁₋₃-alkyl): phenylalkyl optionally substituied with one or two groups which are independently halo, haloalkyl, alkoxy, amino, alkylamino, or dialkylamino; —NR¹¹R^{11a}; heterocycloalkyl; cycloalkylalkyl; alkyl substituted with one or two R¹⁶; or hydroxyalkyl; where each R¹¹ is independently hydrogen or alkyl (in another embodiment each alkyl is C₁₋₃-alkyl); each R^{11a} is independently alkyl (in another embodiment each alkyl is C₁₋₃-alkyl), phenyl opltionally substituted with alkoxy, or is heterocycloalkyl optionally substituted with one or two alkyl; each R¹⁶ is independently halo, amino, alkylamino, dialkylamino, or cyclopropylamino; and all other groups are independently as defined in the Summary of the Invention for a Compound of Formula I or as defined in embodiment (1).

### Embodiments (D3)

In another embodiment, R² is according to Formula (a) where R³ is hydrogen, halo, alkyl, cycloalkylalkyl, or phenylalkyl optionally substituted with one or two R¹⁹; R^{3a} is hydrogen, alkyl, halo, oplionally substituted heterocycloalkyl, or —NR¹¹R^{11a}; and R^{3h} is hydrogen, alkyl, hydroxyalkyl, cyanoalkyl, or alkyl substituted with one or two R¹⁶; and all other groups are independently as defined in the Summary of the Invention for a Compound of Formula I or asd efined in embodiment (I).

### Embodiments (D3a)

In another embodiment, R² is according to Formula (a) where R¹ is phenylalkyl optionally substituted with one or two R¹⁹; R^{3a} is alkyl; and R^{3b} is hydrogen, alkyl, hydroxyalkyl, or alkyl substituted with one R¹⁶; and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ is independently halo, alkyl, haloalkyl, alkoxy, amino, alkylamino, or dialkylamino; R^{3a} is alkyl (in another embodiment alkyl is C₁₋₃-alkyl); and R^{3h} is hydrogen, alkyl, hydroxyalkyl, or alkyl substituted with one R¹⁶; R¹⁶ is amino, alkylamino, dialkylamino, cyclopropylamino, or —OC(O)C₃; and all Other groups are independently as defined in the Summary of the Invention for a Compound of Formula I or as defined in embodiment (1).

### Embodiments (D3a)

In another embodiment, R² is according to Formula (a) where R³ is phenylalkyl optionally substituted with one or two R¹⁹; R^{3a} and R^{3b} are alkyl; and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ are independently halo, alkyl, haloalkyl, amino, alkylamino, dialkylamino, or alkoxy; R^{3a} and R^{3b} are alkyl (in another embodiment each alkyl is C₁₋₃-alkyl); and all other groups are independently as defined in the Summary of the Invention for a Compound of Formula I or as defined in embodiment (1). In another embodiment. R² is according to Formula (a) where R³ is phenylalkyl optionally substituted with one or two halo; R^{3a} and R^{3b} are alkyl (in another embodiment each alkyl is C₁₋₂alkyl); and all other groups are independently as defined in the Summary of the Invention for a Compound of Formula I or as defined in embodiment (1). In another embodiment, R² is according to Formula (a) where R³ is phenylalkyl optionally substituted with one or two R¹⁹; each R¹⁹ are independently halo, alkyl, haloalkyl, amino, alkylamino, dialkylamino, or alkoxy; R^{3a} and R^{3b} are methyl; and all other groups are independently as defined in the Summary of the Invention for a Compound of Formula I or as defined in embodiment (1).

### Embodiments (D3c)

In another embodiment,. R² is according, to Formida (a) where R³ and R³a are alkyl (in another embodiment each alkyl is C₁₋₃-alkyl); R^{3b} is hydrogen, alkyl, or alkyl substituted with one R¹⁶; and all other groups are independently as defined in the Summary of the Invention for a Compound of Formula I or as defined in embodiment (1). In another another embodiment, R² is according io Formula (a) where R³ and R^{3a} are alkyl (in another embodiment alkyl is C₁₋₃alkyl); R^{3b} is hydrogen; and all other groups are independently as denned in the Summary of the Invention for a Compound of Formula I or as defined in embodiment (1). In another embodiment, R² is according to Formula (a) where R³, R^{3a}, and R^{3b} are alkyl (in anotber embodiment each alkyl is C₁₋₂-alkyl); and all other groups are independently as defined in the Summary of the Invention for a Compound of Formula I or as defined in embodiment (I). In another embodimentiu, R² is according to Formula (a) where R³ and R^{3a} are alkyl (in another embodiment each alkyl is C₁₋₂-alkyl); and R^{3b} is alkyl substituted with one R¹⁶; and all other groups are independently as defined in ihe Summary of the Invention for a Compound of Formula I or as defined in embodiment (1). In another embodiment. R² is according to Formula (a) where R³ and R^{3a} are alkyl (in another embodiment each alkyl is C₁₋₂-alkyl); and R^{3b} is alkyl substituted with one R¹⁶; R¹⁶ is amino, alkylamino, dialkylamino, or cycloalkylamino; and all other groups are independently as defined in the Summary of the Invention for a Compound of Formula I or as defined in embodiment (1).

### Embodiments (D3d)

In another embodiment, R² is according to Formula (a) where R³ is alkyl; R^{3a} and R^{3b} are hydrogen; and all oiher groups are independently as defined in the Summary of the Invention for a Compound of Formula I or as defined in embodiment (1). In another embodiment, R² is according to Formula (a) where R³ is C₁₋₂-alkyl; R^{3a} and R^{3b} hydrogen; and all othergroups are independently as defined in the Summary of the Invention for a Compound of Formula I or as defined in embodiment (1).

### Embodiments (D3e)

In another embodiment, R² is according to Formula (a) where R³ is phenylalkyl optionally substituted with one or two R¹⁹; R^{3a} is alkyl; and R^{3b} is hydrogen; and all other groups are independently as defined in the Summary of the Invention for a Compound of Formula I or as defined in embodiment (1). In another embodiment R² is according to Formula (a) where R³ is phenylalkyl optionally substituted with one or two R¹⁹; each R¹⁹ is independently halo, alkyl, haloalkyl, amino, alkylamino, dialkylamino, or alkoxy; R^{3a} is alkyl; and R^{3b} is hydrogen; and all other groups are independently as defined in the Summary of the Invention for a Compound of Formula I or as defined in embodiment (1).

### Embodiments (D3f)

In another embodiment, R² is according to Formula (a) where R³ is phenylalkyl optionally substituted with one or two R¹⁹; R^{3a} is alkyl; and R^{3b} is alkyl substituted with one R¹⁶; and all other groups are independently as defined in the Summary of the Invention for a Compound of Formula I onas defined in embodiment (1). In another embodiment, R² is according to Formula (a) where R³ is phenylalkyl optionally substituted with one of two R¹⁹; each R¹ is independently halo, alkyl, haloalkyl, amino, alkylamino, dialkylamino, or alkoxy, R^{3a} is alkyl (in another embodiment alkyl is C₁₋₂-alkyl); and R^{3b} is alkyl substituted with one R¹⁶ , R¹⁶ is amino, alkylamino, dialkylamino, or cycloalkylamino; and all other groups are independently as defined in the Summary of the Invention for a Compound of Formula I or as defined in embodiment (1).

### Embodiments (D3g)

In another embodiment, R² is according to Formula (a) where R³ is alkyl or phenylalkyl optionally substituted with one or two R¹⁹, R^{3a} is alkyl; and R^{3b} is hydrogen, alkyl, or alkyl substituted with R¹⁶; and all other groups are independently as defined in the Summary of the Invention for a Compound of Formula I or as defined in embodiment (1). In another embodiment, R² is according to Formula (a) where R³ is alkyl (in another embodiment alkyl is C₁₋₃-alkyl) or phenylalkyl optionally substituted with one or two R¹⁹; R^{3a} is alkyl (in another embodiment alkyl is C₁₋₂-alkyl); and R^{3a} is hydrogen, alkyl (in another embodiment alkyl is C₁₋₃-alkyl), or alkyl substituted with R¹⁶; R¹⁶ is amino, alkylamino, dialkylamino, of cycloalkylamino; each R¹⁹ is independently halo, alkyl, haloalkyl, amino, alkylamino, dialkylamino, or alkoxy; and all other groups are independently as defined in the Summary of the Invention for a Compound of Formula I or as defined in embodiment (I).

### Embodiments (D3h)

In another embodiment, R² is according to Formula (a) where R³ is optionally substituted phenyloxy; R^{3a} is alkyl; and R^{3b} is hydrogen; and all other groups are independently as defined in the Summary of the Invention for a Compound of Formula I or as defined in embodiment (I). In another embodiment, R² is according to Formula (a) where R³ is phenyloxy optionally substituted with one or two groups which groups are independently halo, alkyl, haloalkyl, amino, alkylamino, dialkylamino, or alkoxy; R^{3a} is alkyl (in another embodiment alkyl is C₁₋₂-alkyl); and R^{3b} is hydrogen; and all other groups are independently as defined in the Summary of ihc Invention for a Compound of Formula I or as defined in embodiment (1). In another embodiment, R² is according to Formula (a) where R³ is phenyloxy; R^{3a} is alkyl (in another embodiment alkyl is C₁₋₂-alkyl); and R^{3b} is hydrogen; and all other groups are independently as defined in the Summary of the Invention for a Compound of Formula I or as defined in embodiment (1).

### Embodiments (D3i)

In another embodiment, R² is according to Formula (a) where R³ is optionally substituted cycloalkylalkyl; R^{3a} is alkyl; and R^{3b} is hydrogen or alkyl; and all other groups are independently as defined in the Summary of the Invention for a Compound of Formula I or as defined in embodiment (1). In another embodiment, R² is according to Formula (a) where R³ is cycloalkylalkyl; R^{3a} is alkyl (in another embodiment alkyl is C₁₋₂-alkyl); and R^{3b} is hydrogen or alkyl (in another embodiment alkyl is C₁₋₂-alkyl); and all other goups are independently as defined in the Summary of the Invention for a Compound of Formula I or as in embodiment (1).

### Embodiments (D3j)

In another embodiment, R² is according to Formula (a) where R³ is alkyl; R^{3a} is phenylalkyl optionally substituled with one or two R¹⁹ ; and R^{3b} is hydrogen; and all other groups are independently as defined in the Summary of the Invention for a Compound of Formula I or as defined in embodiment (1). In another embodiment, R² is according to Formula (a) where R¹³ is alkyl (in another embodiment alkyl is C₁₋₂-alkyl); R^{3a}; is phenylalkyl optionally substituted with one or two R¹⁹; each R¹⁹ is independently halo, alkyl, haloalkyl, amino, alkylamino, dialkylamino, or alkoxy; and R^{3b} is hydrogen; and all other groups are independently as defined in the Summary of the Invention for a Compound of Formula I or as defined in embodiment (1). In another embodiment, R² is according to Formula (a) where R³ is alkyl (in another embodiment alkyl is C₁₋₂-alkyl), R^{3a} is phenylalkyl; and R^{3h} is hydrogen; and all other groups are independently as defined in the Summary of the Invention for a Compound of Formula I or as defined in embodiment (1).

### Embodiments (D3k)

In another embodiment, R² is according to Formula (a) where R³ is alkyl; R^{3a} is —NY¹¹R^{11a}; and R^{3b} is hydrogen or alkyl; and all other groups are independently as defined in the Summary of the Invention for a Compound of Formula I on as defined in embodiment (I). In another embodiment, R² is according to Formula (a) where R³ is alky (in another embodiment alkyl is C₁₋₂-alkyl); R^{3a} is —NR¹¹R^{11a}; R^{3b} is hydrogen or alkyl (in another embodiment alkyl is C₁₋₂-alkyl); R¹¹ is hydrogen or alkyl (in another embodiment alkyl is C₁₋₂-alkyl); R^{11a} is alkyl, aminoalkyl, alkylaminoalkyl, dialkylaminoalkyl, optionally substituted heterocycloalkyl, optionally substituted heterocycloalkylalkyl, optionally substituted phenyl, or optionally substituted phenylalkyl; and all other groups, are independently as defined in the Summary of the Inyention for a Compound of Formula I of as defined in embodiment (1). In another embodiment, R² is according to Formula (a) where R³ is alkyl (in another embodiment alkyl is C₁₋₂alkyl); R^{3a} is —NR¹¹R^{11a}; R^{3b} is hydrogen or alkyl (in another embodiment alkyl is C₁₋₂-alkyl); R¹¹ is hydrogen or alkyl (in another embodiment alkyl is C₁₋₂-alkyl); R^{11a} is alkyl, aminoalkyl, alkylaminoalkyl, dialkylaminoalkyl, heterocycloalkyl, heterocycloalkylalkyl (oplionally substituted with one or two alkyl). phenylalkyl, phenyl (optionally substituted with one or two groups which are independently halo, alkyl, haloalkyl, amino, alkylamino, dialkylamino, or alkoxy); and all other groups are independenily as defined in the Summary of the Invention for a Compound of Formula I or as defined in embodiment (I).

### Embodiments (D4)

In another embodiment, R² is according to Formula (a) where R^{3a} is alkyl (in another embodiment alkyl is C₁₋₂-alkyl), or —NR¹¹R^{11a}; R³ and R^{3b} are hydrogen; and all oilier groups are independenity as defined in the Summary of the Invention for a Compound of Formula I or as defined in embodiment (I).

### Embodiments (D4a)

In another embodiment, R² is according to Formula (a) where R^{3a} is alkyl (in another embodiment alkyl is C₁₋₂-alkyl), and R³ and R^{3b} are hydrogen; and all other groups are independently as defined in the Summary of the Invention for a Compound of Formula 1 or as defined in embodiinenl (I).

### Embodimenls (D4b)

In another embodiment, R² is according to Formula(a) where R^{3a}is —NR¹¹R^{11a}; R³ and R^{3b} are hydrogen; and all other groups are independently as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen: R¹¹ is hydrogen or alkyll R^{11a} is optionally substituted phenyl; and all other groups are independenity as defined in the Summary of the Invention for a Compound of Formula I or as defined in embodiment (1). In another embodiment, R² is according to Formula (a) where R^{3a} is —NR¹¹R^{11a}; R³ and R^{3b} are hydrogen; R¹¹ is hydrogen or alkyl (in another embodiment alkyl is C₁₋₂-alky); R^{11a} is phenyl optionaIly substituted with one or two groups which groups are independenily halo, alkyl, haloalkyl, amino, alkylamino, dialkylamino, or alkoxy; and all other groups are independenily as defined in the Summary of the Invention for a Compound of Formula I or as defined in embodiment (1).

### Embodimenls (D5)

In another embodiment, R² is according to Formula (a) where R³ and R^{3a} are hydrogen; R^{3b} is —NR¹¹R^{11a}; and all other groups are independently as defined in the Summary of the Invention for a Compound of Formula I or as defined in embodiment (I). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is optionally substituted phenyl; and all other groups are independently as defined in the Summary of the Invention for a Compound of Formula I or as defined in embodiment (I). In another embodiment, R² is according to Formula (a) where R³ and R^{3a} are hydrogen: R^{3b} is —NR¹¹R^{11a}; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl): R^{11a} is hydrogen, alkyl (in another embodiment alkyl is C₁₋₂-alkyl), or optionally substituted phenyl; and all other groups are independently as defined in the Summary of the Invention for a Compound of Formula I or as defined in embodiment (1). In another embodiment, R² is according to Formula(a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is hydrogen, alkyl (in another embodiment alkyl is C₁₋₂-alkyl), or phenyl optionally substituted with one or two groups which groups are independently halo, alkyl, haloalkyl, amino, alkylamino, dialkylamino, or alkoxy; and all other groups are independently as defined in the Summary of the Invention for a Compound of Formula I or as defined in embodiment (1). In another embodiment, R² is according to Formula (a) where R³ and R^{3a} are hydrogen; R^{3b} is —NR¹¹R^{11a}; R¹¹ is hydrogen or alkyl (in another embodiment alkyl is C₁₋₂-alkyl); R^{11a} is hydrogen, alkyl (in another embbdiment alkyl is C₁₋₂-alkyl), or phenyl; add all other groups are independently as defined in the Summary of the Invention for a Compound of Formula I or as defined in embodiment (1).

### Embodiments (D6)

In another embodiment, R² is according to Formula (a) where R³ is hydrogen; R^{3a} is alkyl (in another embodiment alkyl is C₁₋₂-alkyl) or —NR¹¹R^{11a}; R^{3b} is hydrogen or alkyl (in another embodiment alkyl is C₁₋₂-alkyl); and all other groups are independently as defined in the Summary of the Invention for a Compound of Formula I or as defined in embodiment (1).

### Embodiments (D6a)

In another embodiment, R² is according to Formula (a) where R³ is hydrogen; R^{3a} is alkyl (in another embodiment alkyl is C₁₋₂-alkyl); R^{3b} is hydrogen; and all other groups are independently as defined in the Summary of the Invention for a Compound of Formula I or as defined in embodiment (1).

### Embodiments (D6b)

In another embodiment, R² is according to Formula (a) where R^{3a} —NR¹¹R^{11a}; R³, and R^{3b} are hydrogen; and all other groups are independently as defined in the Summary of the Invention for a Compound of Formula I or as defined in embodiment (I). In another embodiment, R² is according to Formula (a) where R^{3a} is —NR¹¹R^{11a}; R³ and R^{3b} are hydrogen; R¹¹ is hydrogen or alkyl (in another embodiment alkyl is C₁₋₂-alkyl); R^{11a} is hydrogen, alkyl (in another embodiment alkyl is C₁₋₂-alkyl); or optionally substituted phenyl; and all other groups are independently as defined in the Summary of the Invention for a Compound of Formula I or as defined in embodiment (I). In another embodimeni, R² is according to Formula (a) where R^{3a} is —NR¹¹R^{11a}; R³ and R^{3b} are hydrogen; R¹¹ is hydrogen or alkyl (in another embodiment alkyl is C₁₋₂- alkyl); R^{11a} is hydrogen, alkyl (in another embodiment, alkyl is C₁₋₂-alkyl), or phenyl optionally substituted with one or two groups which groups are independently halo; alkyl, haloalkyl, amino, alkylamino, dialkylamino, or alkoxy; and all other groups are independently as defined in the Summary of the Invention for a Compound of Formula I or as defined in embodiment (1). In another embodiment. R² is according to Formula (a) where R^{3a} is —NR¹¹R^{11a};: R³ and R^{3b} are hydrogen: R¹¹ is hydrogen or alkyl (in another embodiment alkyl is C₁₋₂-alkyl): R^{11a} is hydrogen, alkyl (in another embodiment each alkyl is C₁₋₂-alkyl), or phenyl optionally substituted with one alkoxy; and all other groups are independently as defined in the Summary of the Invention for a Compound of Formula I or as defined in embodiment (1).

### Embodiments (D6c)

In another embodiment, R² is according to Formula (a) where R³, R^{3a}, and R^{3b} are hydrogen; and all other groups are independently as defined in the Summary of the Invention for a Compound of Formula I or as defined in embodiment (1).

### Embodiments-(D6d)

In another embodiment, R² is pyrimidin-2-yl, pyrimidin-4- yl, 5-(phehylmethyl)-6-methyl-pyrimidin-4-yl, 6-(phenylmethyl)-5-methyl-pyrimidin-4-yl, 5- (1-phenylethyl)-6-methyl-pyrimidin-4-yl, 2,6-dimethyl-5-(phenylmethyl)-pyrimidin-4-yl, 5-(phenylmethyl-)-6-ethyl-pyrimidin-4-yl, 2-methyl-pyrimidin-4-yl, 5-methyl-pyrimidin-4-yl, 6-methyl-pyrimidin-4-yl, 5,6-dimethyl-pyrimidin-4-yl, 6-isopropyl-pyrimidin-4-yl, 5-methyl- 6-ethyl-pyrimidin-4-yl, 5-isopropyl-6-methyl-pyrimidin-4-yl, 5-isoamyl-6-methyl-pyrimidin- 4-yl, 5-ethy-6-isopropyl-pyrimidin-4-yl, 5-methyl-6-isopropyl-pyrimidin-4-yl, 5-(phenylmethyl)-6-chloro-pyrimidin-4-yl, 5-(phenylmethyl)-pyrimidin-4-yl, 5-phenyloxy-6- methyl-pyrimidin-4-yl, 5-(cyclopropylmethyl)-6-methyl-pyrimidin-4-yl, 2-amino-pyrimidin- 4-yl, 5-(2-chloro-phenylmethyl)-6-methyl-pyrimidin-4-yl, 5-(3-chloro-phenylmethyl)-6- methyl-pyrimidin-4-yl, 5-(4-chloro-phenylmethyl)-6-methyl-pyrimidin-4-yl, 5-(2-fluoro- phenylmethyl)-6-methyl-pyrimidin-4-yl, 5-(3-fluoro-phenylmethyl)-6-methyl-pyrimidin-4-yl, 5-(4-fluoro-phenylmethyl)-6-methyl-pyrimidin-4-yl, 5-(3,4-difluoro-phenylmethyl)-6- methyl-pyrimidin-4-yl, 5-(3,5-difluoro-phenylmethyl)-6-methyl-pyrimidin-4-yl, 5-(3-chloro- 5-fluoro-phenylmethyl)-6-methyl-pyrimidin-4-yl, 5-(1-(3-fluorophenyl)-ethyl)-6-methyl- pyrimidin-4-yl, 2,6-dimethyl-5-(4-fluoro-phenylmethyl)-pyrimidin-4-yl, 5-(2-methyl- phenylmethyl)-6-methyl-pyrimidin-4-yl, 5-(3-methyl-phenylmethyl)-6-methyl-pyrimidin-4- yl, 5-(4-methyl-phenylmethyl)-6-methyl-pyrimidin-4-yl, 5-(4-chloro-3-(dimethylamino)- phenylmethyl)-6-methyl-pyrimidin-4-yl, 5-(2-methoxy-phenylmethyl)-6-methyl-pyrimidin-4- yl, 5-(3-methoxy-phenylmethyl)-6-methyl-pyrimidin-4-yl, 5-(4-methoxy-phenylmethyl)-6- methyl-pyrimidin-4-yl, 2-(phenylamino)-pyrimidin-4-yl, 6-(phenylamino)-pyrimidin-4-yl, 6-(4-methoxy-phenylamino)-pyrimidin-4-yl, 5-methyl-6-(phenylamino)-pyrimidin-4-yl, 5-(2- trifluoromethyl-phenylmethyl)-6-methyl-pyrimidin-4-yl, 5-(3-trifluoromethyl-phenylmethyl)- 6-methyl-pyrimidin-4-yl, 5-(4-trifluoromethyl-phenylmethyl)-6-methyl-pyrimidin-4-yl, or 5- phcnylmethyl-6-irifluoromcthyl-pyriinidin-4-yl; and all other groups are independently as defined in the Summary of the Invention for a Compound of Formula I or as defined in embodiment (1).

### Embodiments (D7)

In another embodiment, R² is pyridinyl substituted with R³, R^{3a}, R^{3b}, and R^{3c}; where R³, R^{3a}, R^{3b}, and R^{3c} and all other groups are independently as defined in the Summary of the Invenlion for a Compound of Formula I or as defined in embodiment (1).

### Embodiments (D7a)

In another—embodiment, R² is pyridinyl substituted with R³, R^{3a}, R^{3b}, and R^{3c} where R³, R^{3a}, R^{3b}, and R^{3c} are independently hydrogen, alkyl, or phenylalkyl optionally substituted with one or two R¹⁹; and all other groups are independently as defined in the Summary of the Invention for a Compound of Formula I or as defined in embodiment (1). In another embodiment, R² is pyridinyl substituted with R³, R^{3a}, R^{3b}, and R^{3c}; where R³, R^{3a}, R^{3b}, and R^{3c} are independently hydrogen, alkyl, phenylalkyl, or phenylalkyl substituted with one or two halo; and all other groups are independently as defined in the Summary of the Invenlion for a Compound of Formula I or as defined in embodiment (1).

### Embodiments, (D7b)

In another embodiment, R²is pyridinyl substituted with R³, R^{3a}, R^{3b}, and R^{3c}; where R³ is alkyl (in another embodiment alkyl is C₁₋₂-alkyl); R³, R^{3a}, R^{3b}, and R^{3c} are hydrogen; and all other groups are independently as defined in the Summary of the Invention for a Compound of Formula I or as defined in embodiment (1).

### Emhodiments (D7c)

In another embodiment, R² is pyridin-2-yl, pyridin-3-yl, pyridin-4-yl, 2-amino-pyridin4-yl, 3-methyl-pyridin-2-yl, 2-methyl-3-(phenylmethyl)- pyridin-4-yl, 3-(2-fluoro-phenylmethyl)-2-methyl-pyridin-4-yl, 3-(3-fluoro-phenylmethyl)-2- methyl-pyridin-4-yl, or 3-(4-fluoro-phenylmethyl)-2-methyl-pyridin-4-yl; and all other groups are independently asdefined in the Summary of the Invention for a Compound of Formula I or as defined in embodiment (1).

### Embodiments (D7d)

In another embodiment, R² is according to Formula (b)

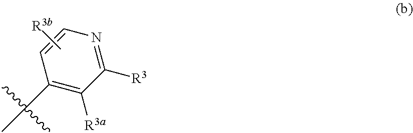

where R³, R^{3a}, and R^{3b} are independently as defined in the Summary of the Invention for a Compound of Formula I or as defined in embodiment (1).

### Embodiments (E)

In another embodiment, R² is a 10-membered heteroaryl substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodimeni (I). In another embodiment, R¹⁰ is a 10-membered heteroaryl and the 10-membered heteroaryl is quinazolin-2-yl, quinazolin-4-yl, quinazolin-5- yl, quinazolin-6-yl, quinazolin-7-yl, quinazolin-8-yl, pyrido[3,2-d]pyiimidin-4-yl, pyrido[4,3- d]pyrimidin-4-yl, pyrido[3,4-d]pyrimidin-4-yl, pyrido[2,3-d]pyrimidin-4-yl, 6,7-dihydro-5H- cyclopental[d]pyrimidin-4-yl, 5,6,7,8-tetrardroquinazolin-4-yl, quinolin-2-yl, quinolin-3-yl, quinolin-4-yl, quinolin-5-yl, quinolin-6-yl, quinolin-7-yl, quiuolin-8-yl, isoquinolin-1-yl, isoquinolin-3-yl, isoquinolin-4-yl, isoquinolin-5-yl, isoquinolin-6-yl, isoquinolin-7-yl, isoquinolin-8-yl, thieno[2,3-d]pyriniidin-4-yl, 7H-pyrrolo[2,3-d]pyrimidinn-4-yl, 1H- pyrrolo[2,3-b]pyridin-4-yl, 1H-pyrrolo[3,2-c]pyridin-4-yl, thieno[2,3-b]pyridin-4-yl, thieno[3;2-c]pyridin-4-yl, 5,7-dihydrothieno[3,4-d]pyrimidin-4-yl, 5,6,7,8- tetrahydropyrido[3,4-d]pyrimidin-4-yl, 5,6,7,8-tetrahydropyrido[4,3-d]pyrimidin-4-yl, 5,6,7,8-tetrahydropyrido[2,3-d]pyrimidin-4-yl, 5,6,7,8-tetrahydropyrido[3,2-d]pyrimidin-4- yl, 6,7-dihydro-5H-pyrrolo[3,4-d]pyrimidin-4-yl, 6,7-dihydro-5H-pyrrolo[3,2-d]pyrimidin-4- yl, 6,7-dihydro-5H-pyriolo[2,3-d]pyrimidin-4-yl, or 5,6-dihydroquinazolinyl where R² is substituted with R³,. R^{3a}, R^{3b1}, R^{3c}, and R^{3d}; where R³, R^{3a}, R^{3b},R^{3c}, and R^{3d}and all other groups are independently as defined in the Summary of the Invention for a Compound of Formula I or as defined in embodiment (1).

### Embodiments (E1)

In another embodiment, R² is quinazolin-2-yl, quinazolin-4- yl, quinazolin-5-yl, quinazolin-6-yl, quinazolin-7-yl, or quinazolin-8-yl, where R² is substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1).

### Embodiments (E2)

In another embodiment, R² is quinazolin-4-yl substituted with R³, R^{3a}, R^{3b}, R^{3c}, and R^{3d}; where R³, R^{3a}, R^{3b}, R^{3c}, and R^{3d} and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; where R³, R^{3a}, R^{3b}, R^{3c}, and R^{3d} are independently hydrogen, halo, alkyl, haloalkyl, alkoxycarbonyl, optionally substituted phenyl, —S(O)₂R²⁰, —NR¹¹R^{11a}, or —OR^{11a}; and all other groups are independently as defined in the Summary of the Invention for a Compound of Formula I or as defined in embodiment (1).

### Embodiments (E2a)

In another embodiment, R² is quinazolin-4-yl substituted with R³, R^{3a}, R^{3b}R^{3c}, and R^{3d}, R^{3e} and R^{3d}are hydrogen and R³, R^{3a}, and R^{3b} are independently cyano, alkyl, alkenyl, halo, haloalkyl, hydroxyalkyl, alkoxyalkyl, —SR¹², —S(O)₂R², —C(O)OR⁴, halocarbonyl, —NR¹¹R^{11a}, —OR^{11a}, optionally-substituled phenyl, optionally-substituted phenylalkyl, optionally substituted cycloalkyl, optimally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}, R^{3e}, and R^{3d} are hydrogen and R³, R^{3a}, and R^{3b} are independently alkyl. halo, or —OR^{11a}; and all other groups are independently as defined in the Summary of the Invention for a Compound of Formula I or as defined in embodiment (1). In another embodiment, R² is quiniazolin-4-yl substituted with R³, R^{3a}, R^{3b}, R^{3c}, and R^{3d}, R^{3e} and R^{3d} are hydrogen and R³, R^{3a}, and R^{3b} are independently alkyl, halo, or —OR^{11a}; R^{11a} is hydrogen, alkyl, or alkoxyalkyl; and all other groups are independently as defined in the Summary of the Invention for a Compound of Formula I or as defined in embodiment (1).

### Embodiments (E2b)

In another embodiment, R² is quinazolin-4-yl substituted with R³, R^{3a}, R^{3b}, R^{3c}, and R^{3d}, R^{3b}, R^{3c}, and R^{3d} are hydrogen, and R³ and R^{3a} are independently cyano, alkyl, alkenyl, halo, haloalkyl, hydroxyalkyl, alkoxyalkyl, —SR¹², —S(O)₂R²⁰, —C(O)OR⁴, halocarbonyl, —NR¹¹R^{11a}, —OR^{11a}, optionally substituted phenyl, optionally substituted phenylalkyl, optionally substituted cycloalkyl, optionally substituted cycloalkylalkyl, optionally substituted heterocyeloalkyl, optionally substituted heterocycloalkylalkyl, optionally substituted heteroaryl, optionally substituted heteroarylalkyl, or alkyl substituted with one or two R¹⁶;and all other groups are independently as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3b}, R^{3c}, and R^{3d} are hydrogen, and R³ and R^{3a} are independently alkyl, halo, —S(O)₂R²⁰, —OR^{11a}, or alkyl substituted with one R¹⁶; and all other groups are independently as defined in the Summary of the Invention for a Compound of Formula I or as defined in embodiment (1). In another embodiment, R² is quinazolin-4-yl substituted with R³, R^{3a}, R^{3b}R^{3c}, and R^{3d}; R^{3h}, R^{3c}, and R^{3d} are hydrogen, and R³ and R^{3a}are independently alkyl, halo, —S(O)₂R²⁰, —OR^{11a}, or alkyl substituted with one R¹⁶; R^{11a} is hydrogen, alkyl, aminoalkyl, alkylaminoalkyl, dialkylaminoalkyl, phenyl, cycloalkylalkyl, phenylalkyl, or heteroaryl: R¹⁶ is amino, alkylamino, dialkylamino, or cycloalkylamino; R²⁰ is alkyl; and all other groups are independently as defined in ihe Summary of the Invention for a Compound of Formula I or as defined in embodiment (1). In another embodiment, R² is quinazolin-4-yl substituted with R³, R^{3a}, R^{3b}, R^{3c}, and R^{3d}, R^{3b}, R^{3c}, and R^{3d}, arec hydrogen, and R³ is —OR^{11a} and R^{3a} is hydrogen, alkyl (in another embodiment alkyl is C₁₋₂-alkyl) or alkyl substituted with one R¹⁶; and all other groups are independently as defined in the Summary of the Invention for a Compound of Formula I or as defined in embodiment (1). In another embodiment R² is quinazolin-4-yl substituted with R³, R^{3a}, R^{3b}, R^{3c}, and R^{3d}, R^{3b, R}^{3c}, and R^{3d} are hydrogen, and R³ is —OR^{11a}and R^{3a} is hydrogen, alkyl, or alkyl substituted with one R¹⁶; R^{11a} is hydrogen or alkyl; R¹⁶ is amino, alkylamino, dialkylamino, or cycloalkylamino; and all other groups are independently as defined in the Summary of the Invention for a Compound of Formula I or as defined in embodiment (1).

### Embodiments (E2c)

In another embodiment, R² is quinazolin-4-yl substituted with R³, R^{3a}, R^{3b}, R^{3c}, and R^{3d}; R^{3a}, R^{3h}, R^{3c}, and R^{3d} are hydrogen and R³ is cyano, alkyl, alkenyl, halo, haloalkyl, hydroxyalkyl, alkoxyalkyl, —SR¹², —S(O)₂R²⁰, —C(O)OR⁴, halocarbonyl, —NR¹¹R^{11a}, —OR^{11a}, optionally substituted phenyl, optionally substituted phenylalkyl, optionally substiiuied cycloalkyl., optionally substituted cycloalkylalkyl, optionally substituted heterocycloalkyl, optionally substituted heterocycloalkylalkyl, optionally substituted heteroaryl, optionally substituled heteroarylalkyl, or alkyl substituted with one or two R¹⁶; and all other groups are independently as defined in the Summary of the Invention for a Compound of Formula I or asdefined in embodiment (1). In another embodiment, R² is quinazolin-4-yl substituted with R³,. R^{3a}; R^{3b}, R^{3c}, and R^{3d}; R^{3a}, R^{3b}, R^{3c}, and R^{3d} are hydrogen and R³ is alkyl, halo, haloalkyl, alkylsullfonyl, optionally substituted phenyl, carboxy, alkoxycarbonyl, —NR¹¹R^{11a}, alkyl substituted with one R¹⁶, or —OR^{11a}a; and all other groups are independenily as defined in the Summary of the Invention for a Compound of Formula I or as defined in embodiment (1). In another embodiment, R² is quinazolin-4-yl substituted with R³, R^{3a}, R^{3b}, R^{3c}, and R^{3d}; R^{3a}, R^{3b}, R^{3c}, and R^{3d} are hydrogen and R³ is alkyl, halo, haloalkyl, alkylsulfonyl, phenyl, carboxy, alkoxycarbonyl. —NR¹¹R^{11a}, alkyl substituted with one R¹⁶, or —OR^{11a}; R¹¹ is hydrogen or alkyl; R^{11a} is hydrogen, alkyl, alkoxyalkyl, cyanoalkyl, or optionally substituted phenylalkyl; R¹⁶ is amino, alkylamino, dialkylamino, or cycloalkylamino; and all other groups are independently as defined in the Summary of the Invention for aCompound of Formula I or as defined in embodiment (1). In another embodiment, R² is quinazolin-4-yl substituted with R³, R^{3a}, R^{3b}, R^{3c}, and R^{3d}; R^{3a}, R^{3b}, R^{2c}, and R^{3d} are hydrogen and R³ is methyl, ethyl, n-propyl, isopiopyl, n-butyl, sec-butyl, isoamyl, bromo, chloro, fluoro, iodo, trifluoromethyl, methylsulfonyl, phenyl, methoxycarbonyl, ethoxycarbonyl, amino, methylamino, ethylamino, n-propylamino, isopropylamino, dimethylamino, diethylamino, N-methyl-N-ethylamino, hydroxy, methoxy, ethyloxy, n-propoxy, isopropoxy, n-butyloxy, sec-butyloxy isoamyloxy, 2-amino-ethyloxy, 2-(methylamino)-ethyloxy, 2-(dimethylamino)-ethyloxy, 3-amino-propyloxy, 3-(methylamino)-propyloxy, 3-(dimethylamino)-propyloxy, 2-methoxy-ethyloxy, cyanomethyloxy, and benzyloxy; and all other groups are independently as defined in the Summary of the Invention for a Compound of Formula I or as defined in embodiment (1).

### Embodiments (E2d)

In another embodiment, R² is quinazolin-4-yl, pyrido[3,2- d]pyrimidin-4-yl, pyrido[4,3-d]pyrimidin-4-yl, pyrido[3,4-d]pyrimidin-4-yl, pyrido[2,3- d]pyrimidin-4-yl, 2-methyl-quinazolin-4-yl, 6-methyl-quinazolin-4-yl, 7-methyl-quinazolin- 4-yl, 8-methyl-quinazolin-4-yl, 2-ethyl-quinazolin-4-yl, 2-phenyl-quinazolin-4-yll 7- (quinolin-2-ylmethyloxy)-8-methoxy-quinazolin-4-yl, 7-(2-dimethylamino-ethyloxy)-8- methoxy-quinazolin-4-yl, 6-3-dimethylamino-propyloxy)-8-methoxy-quinazolin-4-yl, 7- (cyclopropylmethyloxy)-8-methoxy-quinazolin-4-yl, 6-(cyanomethyloxy)-quinazolin-4-yl, 6- methoxy-quinazolin-4-yl, 7-methoxy-quinazolin-4-yl, 8-methoxy-quinazolin-4-yl, 6-ethoxy- quinazolin-4-yl, 6-(n-propoxy)-quinazolin-4-yl, 6,-dimethoxy-quinazolin-4-yl, 7,8- dimethoxy-quinazolin-4-yl, 7-isoamyloxy-8-methoxy-quinazolin-4-yl, 5-bromo-quinazolin-4- yl, 6-bromo-quinazolin-4-yl, 7-bromo-quinazolin-4-yl, 8-bromo-quinazolin-4-yl, 5-chloro- quinazolin-4-yl, 6-chloro-quinazolin-4-yl, 7-chloro-quinazolin-4-yl, 8-chloro-quinazolin-4-yl, 5-fluoro-quinazolin-4-yl, 6-fluoro-quinazolin-4-yl, 7-fluoro-quinazolin-4-yl, 8-fluoro- quinazolin-4-yl, 5-iodo-quinazolin-4-yl, 6-iodo-quinazolin-4-yl, 7-iodo-quinazdlin-4-yl, 8-iodo-quinazolin-4-yl, 6-bromo-7-chloro-quinazolin-4-yl, 6-iodo-7-chloro-quinazolin-4-yl, 6,8-dichloro-quinazolin-4-yl, 6,7-difluoro-quinazolin-4-yl, 6,8-dibromo-quinazolin-4-yl. 2- methyl-7-methoxy-quinazolin-4-yl, 2-ethyl-7-methoxy-quinazolin-4-yl, 2-methyl-6,7- dimethoxy-quinazolin-4-yl, 6-iodo-7-methoxy-quinazolin-4-yl, 6-chloro-7-methoxy- quinazolin-4-yl, 2-chloro-6-methoxy-quinazolin-4-yl, 6-bromo-7-methoxy-quinazolin-4-yl, 7-bromo-8-methoxy-quinazolin-4-yl, 7-bromo-6-methoxy-quinazolin-4-yl, 6-chloro-7.8- dimethoxy-quinazolin-4-yl, 6.7,8-trimethoxy-quinazolin-4-yl, 6-(2-methoxy-ethyloxy)- quinazolin-4-yl, 6-(benzyoxy)-quinazolin-4-yl, 6-hydroxy-quinazolin-4-yl, 7-(benzyoxy)-8- methoxy-quinazolin-4-yl, 7-hydroxy-8-methoxy-quinazolin-4-yl, 7-(benzyoxy)-6-methoxy- quinazolin-4-yl, 7-hydroxy-6-methoxy-quinazolin-4-yl, 6-iodo-8-methyl-quinazolin-4-yl, 6- methyl-8-bromp-quinazolin-4-yl, 2-ethoxycarbonyl-quinazolin-4-yl, 2-methylamino- quinazolin-4-yl, 2-ethylamino-quinazolin-4-yl, 2-(diethylamino)-quinazolin-4-yl, 2- (trifluotomethyl)-quinazolin-4-yl, 7-(trifluoromethyl)-quinazolin-4-yl, 8-(trifluoromethyl)- quinazoIin-4-yl, 6-methylsulfonyl-quinazolin-4-yl, 7-methylsulfonyl-quinazolin-4-yl, quinazolin-4-yl, quinazolin-4-yl, or quinazolin-4-yl: and all other groups are independently as defined in the Summary of the Invention for a Compound of Formula I or as defined in embodiment (1).

### Embodiments (E2e)

In another embodiment, R² is pyrido[3,2-d]pyrimidin-4-yl: and all other groups are independently as defined in the Summary of the invention for a Compound of Formula I or as defined in embodiment (1).

### Embodinierils (E3)

In another embodiment, R² is 5,6,7,8-tetrahydroquinazolin-4- yl, 6,7-dihydro-5H-cyclopentald]pyrimidin-4-yl, 6,78,9-tetrahydro-5H- cyclohepta[d]pyrimidin-4-yl, 5,6-dihydroquinazolin-4-yl, 7′,8′-dihydro-5′H- spiro[cyclopropane-1,6′-quinazoline[-4′-yl, or 6′,8′-dihydro-5′H-spiro[cyclopropane-1,7′- quinazoline-4′-yl where R² is substituted with R³, R^{3a}, R^{3b}, R^{3c}, and R^{3d}, where R³, R^{3a}, R^{3b}, R^{3c}, and R^{3d} and all other groups are independently as defined in the Summary of the Invention for a Compound Formula I or as defined in embodiment (1). In another embodiment, R² is 5,6,7,8-tetrahydroquinalin-4-yl, 6,7-dihydro-5H- cyclopental[d]pyrimidin-4-yl, 6,7,8,9tetrahydro-5H-cycloheptal[d]pyrimidin-4-yl, 5,6- dihydroquinazolin-4-yl, 7′,8′-dihydro-H-spiro[cyclopropane-1.6′-quianzoline]-4′-yl, or 6′,8′- dihydro-5′H-spiro[cyclopropane-1,7′-quinazoline]-4- yl where R² is substituted with R³, R^{3a}, R^{3b}, R^{3c}, and R^{3d}; where R³, R^{3a}, R^{3b}, R^{3c}, and R^{3d} are hydrogen; and all other groups are independently as defined in the Summary of the Invention for a Compound of Formula I or as defined in embodiment (1).

### Enibodiments (E3a)

In another enibodiment, R² is 5,6,7,8-tetrahydroquinazolin- 4yl, 6,7-dihydro-5H-cyclopental[d]pyrimidin-4-yl, 6,7,8,9-5H- cyclohepta[d]pyrimidiin-4-yl, 5,6-dihydroquinazolin-4-yl, or 7′,8′-dihydro-5H- spiro[cyclopropan-1,6′-quinazoline]-4′yl, where R² is substituted with R³, R^{3a},R^{3b}, R^{3c}, and R^{3d}: where R³, R^{3a}, R^{3b}, R^{3c}, and R^{3d} are independently hydrogen, alkyl, alkenyl, halo, haloalkyl, hydroxyalkyl, cyanoalkyl, —SR¹², optionally substituted phenyl, —OR^{11a}, alkyl substituted with one R¹⁶, optionally substituted heterocycloalkyl, optionally substituted heterocycloalkylalkyl, or optionally substituted heteroaryl; and all other groups are independently as defined in the Summary of the Invention for a Compound of Formula or as defined in embodiment (1). In another embodiment R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin- 4-yl, 5,6-dihydroquinazolin-4-yl, or 7′,8′-dihydro-5′H-spiro[cyclopropane-1,6′-quinazoline]- 4′-yl, where R² is substituted with R³, R^{3a}, R^{3b}, R^{3c}, and R^{3d}; where R³, R^{3a}, R^{3b}, R^{3c}, and R^{3d} are independenily hydrogen, alkyl, alkenyl, halo, haloalkyl, hydroxyalkyl, cyanoalkyl, —SR¹², phenyl, —OR^{11a}, alkyl substituted with one R¹⁶, heterocycloalkyl (optionally substituted with alkoxycarbonyl, phenylalkyloxycarbonyl, or alkyl), heterocycloalkylalkyl (optionally substituted with one or two halo), or heteroaryl; R¹² is alkyl or phenylalkyl; R¹⁶ is NR¹¹R^{11a}, —NR¹⁵S(O)R^{15a}, —OR¹⁸, or —OC(O)R¹⁷; R¹¹ is hydrogen or alkyl each R^{11a} is independently hydrogen alkyl, haloalkyl, alkoxyalkyl, carboxyalkyl, cycloalkyl, or cycloalkylalkyl; and all other groups are independently as defined in the Summary of the Invention for a Compound of Formula I or as defined in embodiment (1).

### Embodiments (E3b)

In another embodiment, R² is 5,6,7,8-tetrahydroquinazolin- 4-yl, 6,7-dihydro-5H-cyclopental[d]pyrimidin-4-yl, 6,7,8,9-tetrahydro-5H- cycloheptal[d]pyrimidin-4-yl, 5,6-dihydroquinazolin-4-yl, or 7′,8′-dihydro-5′H- spiro[cyclopropane-1,6′-quinazoline]-4′-yl, where R² is substituted with R³; R^{3a}, R^{3b}, R^{3c}, and R^{3d}; where R^{3a}, R^{3b}, R^{3c}, and R^{3d}are hydrogen, and R³ and all other groups are independently as defined in the Summary of the Invention for a Compound of Formula I or as defined in embodiment (1). In another embodiment, R² is 5,6,7,8-tetrahydroquinazolin-4-yl, 6,7- dihydro-5H-cyclopenta[d]pyrimidin-4-yl, 6,7,8,9-tetrahydro-5H-cyclohepta[d]pyrimidin-4-yl, 5,6-dihydroquinazolin-4-yl, or 7′,8′-dihydro-5H-spiro[cyclopropane-1,6′-quinazoline]-4′-yl, where R² is substituted with R³, R^{3a}, R^{b}, R^{3c}, and R^{3d}; where R^{3a}, R^{3b}, R^{3c}, and R^{3d} are hydrogen, and R³ is alkyl, alkenyl, hydroxyalkyl, alkoxyalkyl, haloalkyl, optionally substituted phenyl, alkyl substituted with one R¹⁶, or —SR¹²; and all other groups are independently as defined in the Summary of the Invention for a Compound of Formula I or as defined in embodiment (1). In another embodiment, R² is 5,6,7,8-tetrohydroquinazolin-4-yl, 6,7-dihydro-5H-cyclopenta[d]pyrimidin-4-yl, 6,7,8,9-tetrahydro-5H-cyclohepta[d]pyrimidin- 4-yl, 5,6-dihydroquinazolin-4-yl, 7′,8′-dihydro-5H-spiro[cyclopropane-1,6′-quinazoline]-4′- yl, where R² is substituted with R³, R^{3a}, R^{3b}, R^{3c}, and R^{3d}; where R^{3a}, R^{3b}, R^{3c}, and R^{3d} are hydrogen, and R³ is alkyl, alkenyl, hydroxyalkyl, alkoxyalkyl, haloalkyl, phenyl, alkyl substituted with one R¹⁶, or —SR¹²; R¹¹ is alkyl or optionally substituted phenylalkyl; and all other groups are independently asdefined in the Summary of the Invention fora Compound of Formula I or as defined in embodiment (1).

### Embodiments (E3c)

In another embodiment, R² is 5,6,7,8-tetrahydroquinazolin- 4-yl, 6,7-dihydro-5H-cyclopenta[d]pyrimidin-4-yl, 6,7,8,9-tetrahydro-5H- cyclohepta[d]pyrimidin-4-yl, 5,6-dihydroquinazolin-4-yl, or 7′,8′-dibydro-5H- spiro[cycloopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, and R³ and R^{3a} are independently alkyl, halo, optionally substituted phenyl, —SR¹², or alkyl substituted with one R¹⁶; and all other groups are independenily as defined in the Summary of the Invention for a Compound of Formula I or as defined in embodiment (1). In another embodiment, R² is 5,6,7,8-tetrahydroquinazolin- 4-yl, 6,7-dihydro-5H-cyclopenta[d]pyrimidin-4-yl, 6,7,8,9-tetrahydro-5H- cyclohepta[d]pyrimidin-4-yl, 5,6-dihydroquinazolin-4-yl, or 7′,8′-dihydro-5H- spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} , are hydrogen, and R³ and R^{3a} are independently alkyl, halo, phenyl, alkyl substituted with one R¹⁶, or —SR¹²; R¹² is alkyl or phenyl; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin- 4-yl, 5,6-dihydroquinazolin-4-yl, or 7′,8′-dihydro-5H-spiro[cyclopropane-1,6-quinazoline]- 4′-yl, where R² is substiuted with R³, R^{3a}, R^{3b}, R^{3c}, and R^{3d}; where R^{3b}, R^{3c},R^{3d} are hydrogen. R³ is alkyl (in another embodiment alkyl is C₁₋₂-alkyl), and R^{3a} is alkyl (in another embodiment alkyl is C₁₋₂-alkyl), halo, phenyl, alkyl substituted with one R¹⁶, or —SR¹²; R¹² is alkyl or phenyl; and all other groups are independently as defined in the Summary of the Invention for a Compound of Formula I or as-defined in embodiment (1). In another embodiment. R² is 5,6,7,8-tetrahydroquinazolin-4-yl, 6,7-dihydro-5H- cyclopental[d]pyrimidin-4-yl, 6,7,8,9-tetrahydro-5H-cyclohepta[d]pyrimidin-4yl, 5,6- dihydroquinazolin-4-yl, or 7′,8′-dihydro-5H-spiro[cyclopropane-1,6′-quinazoline]-4′-yl, where R² is substituted with R³, R^{3a}, R^{3b}, R^{3c}, and R^{3d}; where R^{3b}, R^{3c}, R^{3d} are hydrogen, R³ and R^{3a} are alkyl, (in another embodiment each alkyl is C₁₋₂-alkyl); and all other groups are independently as defined in the Summary of the Invention for a Compound of Formula I or as defined in embodiment (1). In another embodiment. R² is 5,6,7,8-tetrahydioquinazolin-4-yl, 6,7-dihydro-5H-cyclopenta[d]pyrimidin-4-yl, 6,7,8,9-tetrahydro-5H-cyclohepta[d]pyrimidin- 4-yl, 5,6-dihydroquinazolin-4-yl, or 7′,8′-dihydro-5H-spiro[cyclopropane-1,6′-quinazoline]- 4′-yl, where R² is substituted with R³, R^{3a}, R^{3b}, R^{3c}, and R^{3d}; where R^{3b}, R^{3c}, R^{3d} are hydrogen, R³ and R^{3a} are halo: and all other groups are independently as defined in the Summary of the Invention for a Compound of Formula I or as defined in embodiment (1). In another embodiment. R² is 5,6,7,8-tetrahydroquinazolin-4-yl, 6,7-dihydro-5H- cyclopenta[d]pyrimidin-4-yl, 6,7,8,9-tetrahydro-5H-cyclohepta[d]pyrimidin-4-yl, 5,6- dihydroquinazolin-4-yl, or 7′,8′-dihydro-5′H-spiro[cyclopropane-1,6′-quinazoline]-4′-yl, where R² is substituted with R³, R^{3a}, R^{3b}, R^{3c}, and R^{3d}: where R^{3b}, R^{c}, R^{3d} are hydrogen, R³ is alkyl (in another embodimeni alkyl is C-alkyl). and R³:i is hydrogen, alkyl. or alkyl substituted wiih R¹⁶; and all other groups are independently as defined in the Summary of the Invention for a Compound of Formula I or as defined in embodiment (1).

### Embodiments (E3d)

In another embodiment, R² is 5,6,7,8-tetrahydroquinazolin- 4-yl, 6-dihydro-5H-cyclopenta[d]pyrimidin-4-yl, 6,7,8,9-tetrahydro-5H- cycloheptal[d]pyrimidin-4-yl, 5,6-dihydroquinazolin-4-yl, or 7′,8′-dihydro-5H- spiro[cyclopropane-1,6′-quinazoline]-4′-yl, where R² is substituted wiih R³, R^{3a}, R^{3b}, R^{3c} , and R^{3d}; where R^{3c}, R^{3d} are hydrogen, and R³, R^{3a}, and R^{3b} are independently alkyl, alkenyl, halo, hydroxyalkyl, cyanoalkyl, alkyl substituted with R¹⁶, heterocycloalkyl, or heterocycloalkylalkyl (optionally substituted with one or two halo); and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d]pyrimidin-4-yl, 6,7,8,9-tetrahydro-5H-cyclohepta[d]pyrimidin- 4-yl, 5,6-dihydroquinazolin-4-yl, or 7′,8′-dihydro-5H-spiro[cyclopropane-1,6′-quinazoline]- 4′-yl, where R² is substituted with R³, R^{3a}, R^{3b}, R^{3c}, and R^{3d}; where R^{3c}, R^{3d} are hydrogen, and R³, R^{3a}, and R^{3b} are independently alkyl, alkenyl, halo, hydroxyalkyl, cyanoalkyl, alkyl substituted with R¹⁶, heterocycloalkyl, or heterocycloalkylalkyl (optionally substituted with one or two halo); R¹⁶ is NR¹¹R^{11a} where R¹¹ is hydrogen or alkyl and R^{11a} is alkyl, haloalkyl, alkoxyalkyl, cycloalkyl, cycloalkylalkyl, or carboxyalkyl; or R¹⁶ is —NR¹⁵S(O)R^{15a} where R¹⁵and R¹³aare independently hydrogen or alkyl; or R¹ is —OC(O)R¹⁷ where R¹⁷ is alkyl: R¹⁶ is —OR¹⁸ where R¹⁸ is alkyl or alkoxyalkyl; and all other groups are independently as defined in the Summary of the Invention for a Compound of Formula I or as defined in embodiment (1).

### Embodimenis (E3e)

In another embodiment, R² is 5,6,7,8-tetrahydroquinazolin- 4-yl, 6,7-dihydro-5H-cyclopenta[d]pyrimidin-4-yl, 6,7,8,9-tetrahydro-5H- cyclohepta[d]pyrimidin-4-yl, 5,6-dihydroquinazolin-4-yl, or 7′,8′-dihydro-5H- spiro[cyclopropaue-1,6′-quinazoliue]-4′-yl, where R² is substituted with R³, R^{3a}, R^{3b}R^{3c}, and R^{3d}; where R^{3c}, R^{3d} are hydrogen, and R³, R^{3a}, and R^{3b} are alkyl (in another embodiment each alkyl is C₁₋₂-alkyl): and all other groups are independently as defined in ihc Summary of the Invention for a Compound of Formula I or as defined in embodiment (1). In another embodiment, R² is 5,6,7,8-tetrahydroquinazolin-4-yl, 6,7-dihydro-5H- cyclopenta[d]pyrimidin-4-yl, 6,7,8,9-tetrahydro-5H-cyclohcepta[d]pyrimidin-4-yl, 5,6- dihydroquinazolin-4-yl, or 7′,8′-dihydro-5H-spiro[cyclopropane-1,6′-quinazoline]-4′-yl, where R² is substituted with R³, R^{3a}, R^{3b}, R^{3c}, and R^{3d}where R^{3c}, R^{3d} are hydrogen, R³ and R^{3a} are alkyl (in another embodiment each alkyl is Ci₁₋₂-alkyl), and R^{3b} is alkyl substituted with R¹⁶; and all other groups are independently as defined in the Summary of the Invention for a Compound of Formula I or as defined in embodiment (1). In another embodiment, R² is 5,6,7,8-tetrahydroquinazolin-4-yl, 6,7-dihydro-5H-cyclopenta[d pyrimidin-4-yl, 6,7,8,9- tetrahydro-5H-cycloheptal[d]pyrimidin-4-yl, 5,6-dihydroquinazolin-4-yl, or 7′,8′-dihydro-5′H- spiro[cyclopropane-1,6′-quinazoline]-4′-yl, where R² is substituted wilh R³, R³, R^{3a}, R^{3b}, R^{3c}, and R^{3d}; where R^{3c}, R^{3d} are hydrogen. R³ and R^{3a} are alkyl (in another embodiment each alkyl is C₁₋₂-alkyl); and R^{3b} is heterocycloalkylalkyl; and all other groups are independenity as defined in the Summary of the Invention for a Compound of Formula I or as defined in embodiment (1). In another embodiment, R² is 5,6,7,8-tetrahydroquinazolin-4-yl, 6,7- dihydro-5H-cyclpental-8 d]pyrimidin-4-yl, 6,7,8,9-tetrahydro-5H-cyclohepta[d]pyrimidin-4-yl, 5,6-dihydroquinazolin-4-yl, or 7′,8′-dihydro-5′H-spiro[cyclopropane-1,6′-quinazoline]-4′-yl, where R² is substituted wilh R³, R^{3a}, R^{3b}, R^{3c}, and R^{3d}; where R^{3c}, R^{3d} are hydrogen, R³ and R^{3a} are alkyl, (in another embodiment each alkyl is C₁₋₂-alkyl), and R^{3h} is heterocycloalkyi: and all other groups are independently as defined in the Summary-of the Inventionfor a Compound of Formula I or as defined iii embodiment (1).

### Embodiments (E3f)

In another embodiment, R² is 6,7-dihydro-5H- cyclopenta[d]pyrimidin-4-yl, 6-methyl-6,7-diliydro-5H-cyclopenta[d]pyrimidin-4-yl, 6,6-dimethyl-6,7-dihydro-5H-cyclopenta[d]pyrimidin-4-yl, 6-methyl-2-(methylthio)-6,7- dihydlro-5H-cyclopenta[d]pyrimidin-4-yl, 2-(ethythio)-6,7-dihydro-5H- cyclopenta[d]pyrimidin-4-yl, 2-(phenylmethylthio)-6,7-dihydro-5H-cyclopenta[d]pyrimidin- 4-yl, 5-phenyl-6,7-dihydro-5H-cyclopental[d]pyrimidin-4-yl, 6-phenyl-6,7-dihydro-5H- cyclopenta[d]pyrimidin-4-yl, -5,6,7,8-tetrahydroquinazolin-4-yl, 6-methyl-5,6,7,8- tetrahydroquinazolin-4-yl, 6-ethyl-5,6,7,8-tetrahydroquinazolin-4-yl, 7-methyl-5,6,78,- tetrahydroquinazolin-4-yl, 7-methyl-7-phenyl-5,6,7,8-tetrahydroquinazolin-4-yl, 6,6-dimethyl-5,6,7,8,-tetrrahydroquinazolin-4-yl, or 7,7-dimethylo-5,6,7,8- tetrahydroquinazolin-4-yl; and all other groups are independently as defined in the Summary of the Invention fora Compound of Formula I or as defined in embodiment (1).

### Embodiments: (E4)

In another embodiment, R² is according to Formula (c)

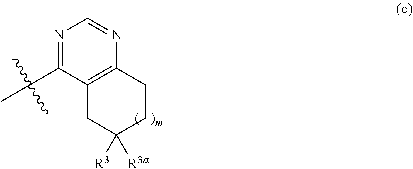

where m is 0 or 1 and R³, R^{3a}, and all other groups are independently as defined in the Summary of the Invention for a Compound of Formula I or as defined in embodiment (1). In another embodiment, R² is according to Formula (c) where m is 0 or 1 and R³ and R^{3a}, together with the carbon to which they are attached, form an oplionally substiiuied cycloalkyl or an optionally substituted heteroeycoalkyl; and all other groups are independently as defined in the Summary of the Invention for a Compound of Formula I or as defined in embodiment (1). In another embodiment, R² is according to Formula (c) where m is 0 or 1 and R³ and R^{3a} are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (c) where m is 0 or 1 and R³ and R^{3a} are halo; and all other groups are independently as defined in the Summary of the Invention for a Compound of Formula I or as defined in embodiment (1).

### Embodiments ( E4a)

In another embodiment, R² is acording to formula (c), m is 1, R³ and R^{3a} are as defined in any of the embodiments (E4d); and all other groups are as defined in the Summary of thednveniion for a Compound of Formula I or as defined in embodiment (1).

### Embodiments (E4b)

In another embodiment, R² is6,6-dimethyl-5,6,7,8- tetrahydroquinazolin-4-yl, 6,6-dichloro-5,6,7,8-tetrahydroquinazolin-4-yl, 6,6-difluoro- 5,6,7,8-tetrahydroquinazolin-4-yl, 7,7-dimethyl-5,6,7,8-tetrahydroquinazolin-4-yl, 7,7- dichloro-5,6,7,8-tetrahydroquinazolin-4-yl, 7′,8′-dihydro-5′H-spiro[cyclopropane-1,6′- quinazoline]-4′-yl, or 6′,8′-dihydro-5′H-spiro[cyclopropane-1,7′-quinazoline]-4′-yl, where R²is substituted with R^{3b} where R^{3b} is hydrogen; alkyl, or haloalkyl; and all other groups are independently as defined in the Summary of the Invention for a Compound of Formula I or as defined in embodimeni (1).

### Embodiments (E4d)

In another embodiment. R² is according to Formula (d)

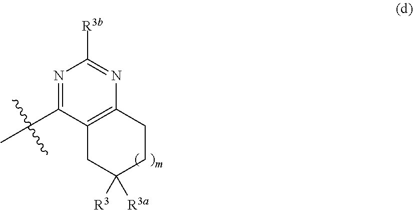

where m is 0 or 1; R³, R^{3a}R^{3b}, and all other groups are independently as defined in the Summary of the Invention for a Compound of Formula I or as defined in embodiment (1). In another embodiment, R² is according to Formula (d) where m is 0 or 1; R³ and R^{3a} are alkyl (in another embodiment each alkyl is C₁₋₂-alkyl); and all other groups are independently as defined in the Summary of the Invention for a Compound of Formula I of as defined in embodiment (1). In another embodiment, R² is according to Formula (d) where m is 0 or 1; R³ and R^{3a} are halo; and all other groups are independently as defined in the Summary of the Invention for a Compound of Formula I or as defined in embodiment (1). In another embodiment, R² is according to Formula (d) where m is 1; R³ and R^{3a}; are alkyl (in another embodiment each alkyl is C₁₋₂-alkyl); and all other groups are independently as defined in the Summary of the Invention for a Compound of Formula I or as defined in embodiment (1). In another embodiment, R² is according to Formula (d) where in is 1; R³ and R^{3a} are halo; and all other groups are independently as defined in the Summary of the Invention for a Compound of Formula I or as defined in embodiment (1). In another embodiment, R² is according to Formula (d) where m is 1: R³ and R^{3a} are alkyl (in another embodiment each alkyl is C₁₋₂-alkyl); R^{3b} is hydrogen, alkyl, alkenyl, hydroxyalkyl, cyanoalkyl, heteorcycloalkyl (optionally substituted with alkoxycarbonyl, benyloxycarbonyl, or alkyl), heteorcycloalkyalkyl (optionally substituted with one or two halo), or alkyl substituted with one R¹⁶; and all other groups are independently as defined in the Summary of the Invention for a Compound of Formula I or as defined in embodiment (1). In another embodiment, R² is according to Formula (d) where m is 1; R³ and R^{3a} are alkyl (in another embodiment each alkyl is C₁₋₂-alkyl); R^{3b} is hydrogen, alkyl, alkenyl, hydroxyalkyl, cyanoalkyl, heteorcycloalkyl (optionally substituted with alkoxycarbonyl,benzyloxycarbonyl, or alkyl), heteorcycloalkylalkyl (optionally substituted with one or two halo), or alkyl substituted with one R¹⁶; R¹⁶is —NR¹¹R^{11a}, —NR¹⁵S(O)₂R^{15a}, —OC(O)R¹⁷, or —OR¹⁸; and all other groups are independently as defined in the Summary of the Invention for a Compound of Formula I or as defined in embodiment (1). In another embodiment, R² is according to Formula (d) where in is 1; R³ and R^{3a} are alkyl (in another embodimeni each alkyl is C₁₋₂-alkyl): R^{3b} is hydrogen, alkyl (in anbther embodiment alkyl is C₁₋₂-alkyl), cyanoalkyl or alkyl substituted with one R¹⁶; and all other groups are independently as defined in the Summary of the Invention for a Compound of Formula I or as defined in embodiment (1).

In another embodiment, the Compound is according to Formula (a), R² is according to embodiments (E4d) and R¹ is according to embodiments (Z)-(Z5).

### Embodiments (E5a)

In another embodiment, R² is according to Formula (e)

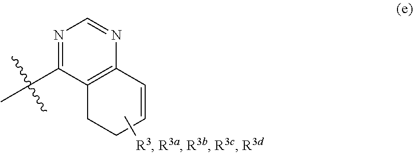

where R³, R^{3a}, R^{3b}, R^{3c}, and R^{3d} are positioned on any substitutable carbon of ring (e); and all other groups are independently as defined in the Summary of the Invention for a Compound of Formula I or as defined in embodiment (1). In another embodiment, R² is according to Formula (e) where oiie of R³, R^{3a}, R^{3b}, R^{3c}, and R^{3d} is hydrogen, alkyl (in another embodiment each alkyl is C₁₋₂-alkyl), or alkyl substituted with one R¹⁶ and the other of R³, R^{3a}, R^{3b}, R^{3c}, and R^{3d} and all other groups are independently as defined in the Summary of the Invention for a Compound of Formula or as defined in embodiment (1). In another embodiment, R² is according to Formula (e) where one of R³, R^{3a}, R^{3b}, R^{3c}, and R^{3d} is hydrogen, alkyl (in another embodiment alkyl is C₁₋₂-alkyl), or alkyl substituted with one R¹⁶ and the other of R³, R^{3a}, R^{3b}, R^{3c}, and R^{3d} are independently hydrogen or alkyl (in another embodiment each alkyl is C₁₋₂-alkyl)l and all other groups are as defined in the Summary of the Invention for a Compound of Formula or as defined in embodiment (1). In another embodiment, R² is according to Fornnila (e), where one of R³, R^{3a}, R^{3b},R^{3c}, and R^{3d} is hydrogen; alkyl (in another embodiment each alkyl is C₁₋₂-alkyl); or alkyl substituted with one R¹⁶ and the other of R³, R^{3a}, R^{3b}, R^{3c}, and R^{3d} are alkyl, (in another embodiment each alkyl is C₁₋₂-alkyl); and all other groups are as defined in the Summary of the Invention for a Compound of Formula or as defined in embodiment (1). In another embodiment, R² is according to Formula (c) where one of R³, R^{3a}, R^{3b}, R^{3c}, and R^{3d} is hydrogen, alkyl (in another embodiment alkyl is C₁₋₂-alkyl), or alkyl substituted with one R¹⁶ , a second of R³, R^{3a}, R^{3b}, R^{3c}, and R^{3d} is hydrogen, and the other of R³, R^{3a}, R^{3b}, R^{3c}, and R^{3d} are alkyl (in another emhodiment each alkyl is C₁₋₂-alkyl); and all other groups are as defined in the Summary of the Invent ion for a Compound of Formulua or as defined in embodiment (1).

In another embodiment, the Compound is according to I (a). R² is according to embodiments (E5a) and R¹ is according to embodiments (Z)-(Z5).

### Embodimenls (E5b)

In another embodiment, R² is according lo Formula (f)

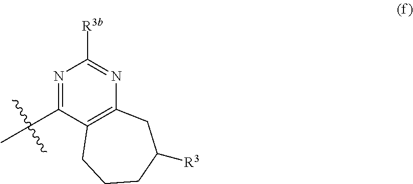

where R^{3b} is hydrogen, alkyl (in another embodiment alkyl is C₁₋₂-alkyl), cyanoalkyl, or alkyl substituted with one R¹⁶; and R³ is hydrogen, alkyl (in another embodiment alkyl is C₁₋₂- alkyl), or alkenyl; and all other groups are as defined in the Summary of the Invention for a Compound of Formula or as defined in embodiment (1).

In another enibodiment, the Compound is according to I (a), R² is according to embodiments (E5b) and R¹ is according to embodimenls (Z)-(Z5).

### Embodiments (E5c)

In another embodiment, R² is according to Formula (g)

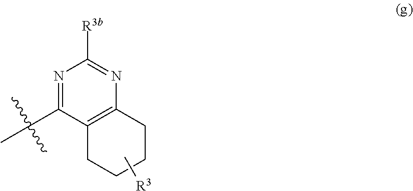

where R^{3b} s hydrogen, alkyl (in another embodiment alkyl is C₁₋₂-alkyl), cyanoalkyl, or alkyl substituted with one R¹⁶; and R³ is alkyl (in another embodiment alkylis C₁₋₂-alkyl): hydroxyalkyl, alkoxyalkyl, or haloalkyl, and is located al the 6- or 7-position of the ring; and all other groups are as defined in the Summary of the Invention for a Compound of Formula or as defined in embodiment (1).

In another embodiment, the Compound is according to Formula I (a), R² is according to embodiments (E5c) and R¹ is according to embodiments (Z)-(Z5).

### Embodiments (E5d)

In another embodiment, R² is according lo Formula (h)

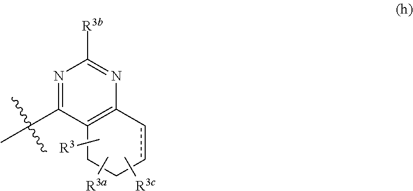

where R³, R^{3a}, R^{3b}, and R^{3c}and all other groups are as defined in the Summary of the Invention for a Compound of Formula or as defined in embodiment (1). In another embodiment, R² is according to Formula (h) where R^{3b} is hydrogen, alkyl, cyanoalkyl, or alkyl substituted with one R¹⁶; and all other groups areas defined in the Summary of the Invention for a Compound-of Formula or as defined in embodiment (1). In another embodiment, R² is according to Formula (h) where R^{3b} is hydrogen, cyanoalkyl, alkyl (in another embodiment alkyl is C₁₋₂-alkyl), or alkyl substituted with one R¹⁶; R³, R^{3a}, and R^{3c} are independently hydrogen, alkyl (in another embodiment alkyl is C₁₋₂-alkyl), alkenyl, halo, haloalkyl, hydroxyalkyl, —SR¹², optionally substituted phenyl, —OR^{a}, alkyl substituted with one R¹⁶, optionally substituted heterocycloalkyl, optionally substituted heterocycloalkylalkyl, or optionally substituted heteroaryl; and all other groups are as defined in the Summary of the Invention for a Compound of Formula or as defined in embodiment (1).

In another embodiment, the Compound is according to Formula I(a), R² is according to embodiments (E5d) and R¹ is according to embodiments (Z)-(Z5).

### Embodiments (E6)

In another embodiment, R² is quinolin-2-yl, quinolin-3-yl, quinolin-4-yl, quinolin-5-yl, quiuolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, or isoquinolin-8-yl, where R² is substituted wilh R³, R^{3a}, R^{3b}, and R^{3c}; where R³, R^{3a}, R^{3b}, and R^{3c} and all other groups are independently as defined in the Summary of the Invention for a Compound of Formula I or as defined in embodiment (1). In another embodiment, R² is quinolin-4-yl or isoquinolin-1-yl, where R² is substituted with R³, R^{3a}, R^{3b}, R^{3c},and R^{3d}; where R³, R^{3a}, R^{3b}, R^{3c}, and R^{3d} and all other groups are independently as defined in the Summary of the Invention for a Compound of Formula I or as defined in embodiment (1).

### Embodiments (E6a)

In another embodiment, R² is quinolin-4-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl, or isoquinolin-8-yl, where R² is substiiuied with R³, R^{3a}, R^{3b}, R^{3c}, and R^{3d}; R^{3b}, R^{3c}, and R^{3d} are hydrogen; R³ and R^{3a} are independently hydrogen, cyano, alkyl, halo, haloalkyl, —OR^{11a}, phenyl, phenylalkyl optionally substituted with one or two R¹⁹, or alkyl substituted with one or two R¹⁶; and all other groups are independently as defined in the Summary of the Invention for a Compound of Formula I or as defined in embodiment (1). In another embodiment, R² is quinolin-4-yl or isoquinolin-1-yl, where R²is substituted with R³, R^{3a}, R^{3b}, R^{3c}, and R^{3d}, R^{3b}, R^{3c},and R^{3d} are hydrogen; R³ and R^{3a} are independenity R³ and R^{3a} are independently hydrogen, cyano, alkyl (in another embodiment alkyl is C₁₋₂alkyl), halo, haloalkyl, —OR^{11a}, phenyl, phenylalkyl optionally substituted with one or two R¹⁹; or alkyl substituted wilh one or two R¹⁶; and all other groups areindependently as defined iii the Summary of the Invention fora Compound of Formula I or as defined in embodiment (1).

### Embodiments (E6b)

In another embodiment, R² is 6,7-dimethoxy-quinolin-4-yl, 7-cyano-quinolin-4-yl, 5-fluoro-quinolin-4-yl, 6-fluoro-quinolin-4-yl, 7-fluoro-quinolin-4-yl, 8-fluoro-quinolin-4-yl, 2-phenyl-quinolin-4-yl, 2-methyl-quinolin-4-yl, 2-methyl-7-methoxy- quinolin-4-yl, 2-trifluoromethyl-quinolin-4-yl, or isoquinolin-1-yl; and all other groups are independently as defined in the Summary of the Invention for a Compound of Formula I or as defined in embodiment (1).

### Embodiments (E7)

In another enibodinient, R² is 5H-pyrrolo[3.2-d]pyrimidin-4- yl, thieno[2,3-d]pyrimidin-4-yl,7H-pyrrolo[2,3-d]pyrimidin-4-yl, 1H-pyrrolo[2.3-b-]pyridin- 4-yl, 1H-pyrrolo[3,2-c]pyridin-4-yl, thieno[2,3-b]pyridin-4-yl, or thieno[3,2-c]pyridin-4-yl, where R² is substituted with R³, R^{3a}, R^{3b}, R^{3c}, and R^{3d}; R³, R^{3a}, R^{3b}, R^{3c}, and R^{3d} and all other groups are independenity as defined in the Summary of the Invention for a Compound of Formula I or as defined in embodiment, (1). In another embodiment, R² is thienol 2,3- [d]pyrimidin-4-yl or 7H-pyrrolo[2,3-d]pyrimidin-4-yl, where R² is substituted with R³, R^{3a}, R^{3b}, R^{3c}, and R^{3d}; R³, R^{3a}, R^{3b}, R^{3c}, and R^{3d} and all other groups are independently as defined in the Summary of the Invention for a Compound of Formula I or as defined in embodiment (1). In another embodiment, R² is thieno[2,3-d]pyrimidin-4-yl or 7H-pyrrolo[2,3- d]pyrimidin-4-yl, where R² is substituted with R³, R^{3a}, R^{3b}, R^{3c}, and R^{3d}; R^{3a}, R^{3b}, R^{3c}, and R^{3d} are hydrogen; R³ is hydrogen or alkyl (in another embodiment alkyl is C₁₋₂-alkyl); and all other groups are independently as defined in the Summary of the Invention for a Compound of Formula I or as defined in embodiment (I). In another embodiment, R² is thieno[2,3- d]pyrimidin-4-yl, 5-methyl-thieno[2,3-d]pyrimidin-4-yl, or 7H-pyrrolo[2,3-d]pyrimidin-4-yl; and all other groups are independently as defined in the Summary of the Invention for a Compound of Formula I or as defined in embodiment (1).

### Embodiments (E8)

In another embodiment, R² is 5,7-dihydrothieno[3,4- d]pyrimidin-4-yl, 5,6,.7-tetrahydropyrido[3,4--d]pyrimidin-4-yl, 5,6,7,8- tetrahydropyrido[4,3-d]pyrimidin-4-yl, 5,6,7,8-tetrahydropyrido[2,3-d]pyriniidin-4-yl, 5,6,7,8-tetrahydropyrido[3,2-d]pyrimidin-4-yl, 6,7-dihydro-5H-pyrrolo[3,4-d]pyrimidin-4-yl, 6,7-dihydro-5H-pyrrolo[3,2-d]pyrimidin-4-yl, or 6,7-dihydro-5H-pyrrolo[2,3-d]pyrimidin-4- yl, where R² is substituted with R³, R^{3a}, R^{3b}, R^{3c}, and R^{3d}; where R³, R^{3a}, R^{3b}, R^{3c}, and R^{3d} and all other groups are independently as defined in the Summary, of the Invention for a Compound of Formula I or as defined in embodiment (1).

### Embodiments (E8a)

In another embodiment, R² is 5,7-dihydrothieno[3,4- d]pyrimidin-4-yl, 5,6,7,8-tetrahydropyrido[3,4-d]pyrimidin-4-yl, 5,6,7,8- tetrahydropyrido[4,3-d]pyrimidin-4-yl, or 6,7-dihydro-5H-pyrrolo[3,4-d]pyrimidin-4-yl, where R² is substituted with R³, R^{3a}, R^{3b}, R^{3c}, and R^{3d}R³, R^{3a}, R^{3b}, R^{3c}, and R^{3d} and all other groups are independently as defined in the Summary of the Invention for a Compound of Formula I or as defined in embodiment (1).

### Embodimenis (E8b)

In another embodiment. R² is 5,7-dihydrothieno[3,4- d]pyrimidin-4-yl, 5,6,7,8-tetrahydropyrido[3,4-d]pyrimidin-4—yl, 5,6,7,8- tetrahydropyrido[4,3-d]pyrimidin-4—yl, or 6,7-dihydro—5H-pyrrolo[3,4-d]pyrimidin-4-yl, where R² is substiiuied with R³, R^{3a}, R^{3b}, R^{3c}, and R^{3d}; R³, R^{3a}, R^{3b}, R^{3c}, and R^{3d} and all other groups are independently as defined in the Summary of ihe Invention for a Compound of Formula I or as defined in embodiment (1). In another embodiment, R² is 5,7- dihydrothieno[3,4-d]pyrimidin-4-yl, 5,6,7,8-tetrahydropyrido[3,4-d]pyrimidin-4-yl, 5,6,7,8- tetrahydropyrido[4,3d]pyrimidin-4-yl, or 6,7-dihydro-5H-pyrrolo[3,4d]pyrimidin-4-yl, where R² is substituted with R³, R^{3a}, R^{3b}, R^{3c}, and R^{3d}, R^{3a}, R^{3b}, R^{3c}, and R^{3d} are hydrogen; R³ is hydrogen, -alkyl (in-another emhodiment alkyl is C₁₋₃-alkyl), haloalkyl, optionally substityted phenyl, optionally substituted phenylalkyl, optionally substituted cycloalkyl, or optionally substituted cycloalkylalkyl; and all other groups are independently as defined in the Summary of the Invention for a Compound of Formula I or as defined in embodiment (1).

### Embodimenls (E8c)

In another embodiment, R² is 5,7-dihydrothieno[3,4- d]pyrimidin-4-yl, 5,6,7,8-tetrahydropyrido[3,4-d]pyrimidin-4-yl, 7-ethyl-5,6,7,8- tetrahydropyrido[3.4-d]pyrimidin-4-yl, 7benzyl-5,6,7,8-tetrahydropyrido[3,4-d]pyrimidin-4- yl, 5,6,7,8-tetrahydropyrido[4,3-d]pyrimidin-4-yl, 6-cycloprppyl-5,6,7,8- tetrahydropyrido[4,3-d]pyiimidin-4-yl, 6,7-dihydro-5H-pyrrolo[3,4-d]jpyrimidin-4-yl,-6-p- tolyl-6,7-dihydro-5H-pyrrolo[3,4-d]pyrimidin-4-yl, or 6-cyclopropyl-6,7-dihydro-5H- pyrrolo[3,4-d]pyrimidin-4-yl: and all other groups are independently as defined in the Summary of the Invention for a-Compound of Formula I or as defined in embodiment (1).

### Embodiments (E9)

In another embodiment, R² is 7H-pyrrolo[2,3-d]pyrimidin-4- yl substituted with R³, R^{3a}, R^{3b}, R^{3c}, and R^{3d}; R^{3a}, R^{3b}, R^{3c}, and R^{3d} are hydrogen; R³ and all other groups are independenily as defined in the Summary of the Invention for a Compound of Formula I or as defined in embodiment (1). In another embodiment, R² is 7H-pyrrolo[2,3- d]pyrimidin-4-yl substituted with R³, R^{3a}, R^{3b}, R^{3c}, and R^{3d}; R³, R^{3a}, R^{3b}, R^{3c}, and R^{3d} are hydrogen; and all other groups are independently as defined in the Summary of the Invention for a Compound of Formula I oOr as defined in embodiment (1).

### Embodimenls (E10)

In another embodiment, R² is 1H-pyrazolo[3,4-d]pyrimidin 4-yl substituted with R³, R^{3a}, R^{3b}, R^{3c}, and R^{3d}; R^{3a}, R^{3b}, R^{3c}, and R^{3d} are hydrogen: R³ and all other groups are independently as defined in the Summary of the Invention for a Compound of Formula I or as defined in embodiment (1). In another embodiment, R² is 1H- pyrazolo[3,4-d]pyrimidin-4-yl substituted with R³, R^{3a}, R^{3b}, R^{3c}, and R^{3d}; R³, R^{3a}, R^{3b}, R^{3c}, and R^{3d} are hydrogen: and all other groups are independenily as defined in the Summary of the Invention for a Compound of Formula I or as defined in embodiment (1),

### Embodiments (E11)

In another cmbodimcni, R² is 6,7,8,9- tetrahydropyrimido[4,5-b[indolizin-4-yl substituted with R³, R^{3a}, R^{3b}, R^{3c}, and R^{3d}; where R³, R^{3a}, R^{3b}, R^{3c}, and R^{3d} and all other groups are independently as defined in ihe Summary of the Invention for a Compound of Formula I or as defined in embodiment (1). In another embodiment. R² is 6,7,8-tetrahydropyrimido[4,5-b]indolizin-4-yl substituted with R³, R^{3a}, R^{3b}, R^{3c}, and R^{3d}; R^{3a}, R^{3b}, R^{3c}, and R^{3d} are hydrogen: R³ is hydrogen or cyano: and all other groups are independently as defined in the Summary of the Invention for a Compound of Formula I or as defined in embodiment (1). In another embodiment, R² is 6,7,8,9- tetrahydropyrimido[4,5-b]indolizin-yl or 10-cyano-6,7,8,9-tetrahydropyrimido[4.5- b ]indolizin-4-yl: and all other groups are independently as defined in the Summary of the Invention for a Compound of Formula I or as defined in embodiment (1).

In another embodiment, the Compound is according to any of embodiments (B) and (H1) and R² is according to any one of embodiments (D)-(D2), (D3)-(D3)-(D3), (D3K)-(D4b)- (D4b), (D5), (Db6-D6d),(D7), (D7d), (E)-(E2), (E2a)-(E2e), (E3)-(E3f), (E4)-(E4d), (E5a)-(E5d), (E6)-(E6b), (E7), (E8)-(E8c), and (E9)-(E11). In another embodiment, the Compound is according to any of embodiments (B) and (H1) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B1)-(B2) and R² is according to any one of embodiments (D)-(D2), (D3)-(D3k), (D4)-(D4b), (D5), (D6-D6d), (D7)-(D7d), (E)-(E2), (E2a)-(E2c), (E3)-(E3f), (E4)-(E4d), (E5a)-(E5d), (E6)-(E6b), (E7), (E8)-(E8c), and (E9)-(E11). In another embodiment, the Compound is according to any of embodiments (B1) and R² is according to any one of embodiments (D2). (D3a)-(D3c), (D3g), (D3i), (E2), (E2b), (E3c), (E4a), (E4d),and (E5a)-(E5d).

In anothier embodiment, the Compound is according to any of embodiments (B3). (B4), (B4a), and (B4b) and R² is according to any one of embodiments (D)-(D2), (D3)-(D3k), (D4)-(D4b), (D5), (D6-D6d), (D7)-D7d), (E)-(E2), (E2a)-(E2c), (E3)-(E3f), (E4)-(E4d), (E5a)-(E5d), (E6)-(E6b), (E7), (E8)-(E8c), and (E9)-(E11). In another embodiment, the Compound is according to any of embodiments (B4a) and R² is according to any one of embodiments (D2), (D3a)-(D3c), (D3g), (D3i), (E2), (E2b), (E3c), (E4a), (E4d), and (E5a)-(E5d).

In another embodimeni. the Compound is according to any of embodiments (B5), (B6), (B7), and (B8) and R² is according to any one of embodiments (D)-(D2), (D3)-(D3k), (D4)-(D4b), (D5), (D6-D6d), (D7)-(D7d), (E)-(E2), (E2a)-(E2c), (E3)-(E3f), (E4)-(E4d), (E5a)-(E5d), (E6)-(E6b), (E7), (E8)-(E8c), and (E9)-(E11). In another embodiment, the Compound is according to any of embodiments (B7) and R² is according to any one of embodiments (D2), (D3a)-(D3c), (D3g), (D3i), (E2), (E2b), (E3c), (E4a), (E4d), and (E5a)-(E5d).

In another embodinient, the Compound is according to any of embodiments (B9)-(B13) and R² is according to any one of embodiments (D)-(D2), (D3)-(D3k), (D4)-(D4b), (D5), (D6-D6d), (D7)-(D7d), (E)-(E2), (E2a)-(E2c), (E3)-(E3f), (E4)-(E4d), (E5a)- E5d), (E6)-(E6b), (E7), (E8)-(E8c), anil (E9)-(E11). In another embodiment, the Compound is according to any of embodiments (B9)-(B313) and R² is according to any one of embodiments (D2), (D3a)-(D3c), (D3g), (D3i), (E2), (E2b), (E3c), (E4a), (E4d), and (E5a)-(E5d).

In another embodiment, the Compound is according to any of embodiments (B16), (B16a)-(B16c), (B17), and (B18) and R² is according to any one of embodiments (D)-(D2), (D3)-(D3k), (D4)-(D4b), (D5), (D6-D6d), (D7)-(D7d), (E)-(E2), (E2a)-(E2c), (E3)-(E3f), (E4)-(E4d), (E5a)-(E5d), (E6)-(E6b), (E7), (E8)-(E8c), and (E9)-(E11 ). In another embodiment, the Compound is according to any of embodiments (B16a)-(B16c) and R² is according to anyone of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11 ). In another embodiment, the Compound,is according to any of embodiments (B16a)-(B16c) and R² is according to any one of embodiments (D2), (D3a)-(D3c), (D3g), (D3i), (E2), (E2b), (E3c), (E4a), (E4d), and (E5a)-(E5d),

In another embodiment, the Compound is according to any of embodiments (B19)-(B29) and R² is according to any one of embodiments (D)-(D2), (D3)-(D3k), (D4)-(D4b), (D5), (D6-D6d),(D7)-(D7d), (E)-(E2), (E2a)-(E2c), (E3)-(E3f), i(E4)-(E4d), (E5a)-(E5d), (E6)-(E6b), (E7), (E8)-(E8c), and (E9)-(E11) .In another embodiment, the Compound is according to any of embodiments (B19)-(B29) and R² is accordiug to any one of embodiments (D2), (D3a)-(D3c), (D3g), (D3i), (E2), (E2b), (E3c), (E4a), (E4d),and (E5a)-(E5d).

In another embodiment, the Compound is according to any of embodiments (C)-(C3) and R² is according to any one of embodiments (D)-(D2), (D3)-(D3k), (D4)-(D4b), (D5), (D6-D6d), (D7)-(D7d), (E)-(E2), (E2a)-(E2c), (E3)-(E3f), (E4)-(E4d), (E5a)-(E5d), (E6)-(E6b), (E7), (E8)-(E8c), and (E9)-(E11). In another embodiment, the Compound is according to any of embodiments (C2) and R² is according to any one of embodiments (D)-(D2), (D3)-(D3k), (D4)-(D4b), (D5), (D6-D6d), (D7)-(D7d), (E)-(E2), (E2a)-(E2e), (E3)-(E3f), (E4)-(E4d), (E5a)-(E5d), (E6)-(E6b), (E7), (E8)-(E8c), and (E9)-(E11), In another embodiment, the Compound is according 10 any of embodiments (C2) and R² is according to any one of embodiments (D2), (D3a)-(D3c), (D3g), (D3i), (E2), (E2b), (E3c), (E4a), (E4d), and (E5a)-(E5d).

### Embodiments Z

In another embodiment, the Compound is that where R¹ is benzimidazol-6-yl optionally substituted with one or two R⁷; and R⁷ is as defined in the Summary of the Invention for a Compound of Formula I or as defined in embodiment (1). In another embodiment, the Compound is that where R¹ is benzimidazol-6-yl optionally substituted with one or two R⁷; each R⁷, when present, is alkyl, haloalkyl. —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is thai where R¹ is benzimidazol-6-yl optionally substituted wilh one or two R⁷; each R⁷, when present. is independently alkyl (in another embodiment alkyl is C₁₋₃-alkyl), haloalkyl, —NR⁸R^{8a}, —NR⁸C(O)OR⁹, or cycloalkyl: R⁸ is hydrogen: R^{8a} is hydrogen, alkyl (in another embodiment alkyl is C₁₋₃-alkyl), or haloalkyl; R⁹ is hydrogen or alkyl (in another embodiment alkyl is C₁₋₂-alkyl).

### Embodiments Z1

In another embodiment the Compound is that where R¹ is thiazlo[5,4-b]pyridin-6-yl or thiazolo[4.5-b]pyridin-6-yl optionally substituted with one or two R⁷; and R⁷ is as defined in the Summary of the Invention for a Compound of Formula I or as defined in embodiment (1). In another embodiment, the Compound is that where R¹ is thiazplo[5,4-b]pyridin-6-yl or thiazolo[4,5-b]pyridin-6-yl optionally substituted with one or two R⁷; each R⁷, when present, is independently alkyl, haloalkyl, —NR⁸R^{8a}, —NR⁸C(O)OR⁹, or cycloalkyl; and R⁸, R^{8a}, and R⁹ are independently as defined in ihe Summary of the Invention for a Compound of Formula I or as defined in embodiment (1). In another embodiment, the Compound is that where R¹ is thiazolo[5,4-b]pyridin-6-yl or thiazolo[4,5-b]pyridin-6-yl optionally substituted with one or two R⁷; each R⁷, when present, is independently alkyl (in another embodiment alkyl is C_{1.3}-alkyl), haloalkyl. —NR⁸R^{8a}, —NR⁸C(O)OR⁹, or cycloalkyl: R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃-alkyl).

### Embodinienis Z2

In another embodiment the Compound is that where R¹ is 1H-imidazo[4,5-b]pyridin-5-yl, 1H-imidazo-8 4,5-b]pyridin-6-yl, 3H-imidazo[4,5-b]pyridinii-5-yl, or 3H-imidazo[4,5-d]pyridin-6-yl where R¹ is optionally substituted with R⁷; and R⁷ is as defined in the Summary of the Invention for a Compound of Formula I or as defined in embodiment (1). In another embodiment, the Compound is that where R¹ is 1H-imidazo[4,5- b]pyridin-5-yl, 1H-imidazo[4,5-b]pyridin-6-yl, 3H-imidazo[4,5-b]pyridin-5-yl, or 3H- imidazo[4,5-b]pyridin-6-yl where R¹ is optionally substiiuied with one or two R⁷; each R⁷, when present, is independently alkyl (in another embodiment alkyl is C₁₋₂-alkyl), haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹ is 1H-imidazo[4,5-b[pyridin-5-yl, 1H-imidazo[4,5-b]pyridim-6-yl, 3H-imidazo[4,5-b]pyridin-5-yl, or 3H-imidazo[4.5-b]pyridin- 6-yl where R¹ is optionally substituted with R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃--alkyl), or haloalkyl; R⁹ is hydrogen or alkyl (in another embodiment alkyl is C₁₋₃-alkyl).

### Embodimenls Z3

In another embodiment, the Compound is that where R¹ is 1H-imidazo[4,5-c]pyridin-6-yl or 3H-imidazo[4,5-c]pyridin-6-yl optionally substituted with one or two R⁷; and R⁷ is as defined in the Summary of the Invention for a Compound of Formula I or as defined in embodiment (1). In another embodiment, the Compound is that where R¹ is 1H-imidazo[4,5-c]pyridin-6-yl or 3H-imidazo[4,5-c]pyridin-6-yl optionally substituted with one or two R⁷; each R⁷, when present, is independently alkyl (in another embodiment alkyl is C₁₋₂-alkyl), haloalkyl, —NR⁸R^{8a}, —NR⁸C(O)OR⁹, or cycloalkyl; and R⁸, R^{8a}, and R⁹ are independently as defined in the Summary of the Invention for a Compound of Formula I or as defined in embodiment (1). In another embodiment, the Compound is that where R¹is 1H-imidazo[4,5-c]pyridin-6-yl or 3H-imidazo[4,5-c]pyridin-6-yl optionally substituted with one or two R⁷; each R⁷, when present, is independently alkyl (in another embodiment alkyl is C₁₋₂alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₂alkyl), or haloalkyl; R⁹ is hydrogen or alkyl (in another embodiineni alkyl is C₁₋₃-alkyl).

### Embodiments Z4

In another embodiment, the Compound is that where R¹ is benzol[d]thiazol-5-yl or benzo[d]thiazol-6-yl optionally substituted with one or two R⁷; and R⁷ is as defined in the Summary of the Invention for a Compound of Formula I or as defined in embodiment (1). In another embodiment, the Compound is that where R¹ is benzo[d]thiazol-5-yl or benzo[d]thiazol-6-yl optionally substituted with one, or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; and R⁸, R^{8a}, and R⁹ are independently as defined in te Summary of the Invention for a Compound of Formula I or as defined in embodiment (1). In another embodiment, tge Compound is that where R¹ is benzo[d]thiazol- 5-yl or benzo[d]thiazol-6-yl optionally substituted with one or two R⁷; each R⁷, when present, is independently alkyl (in another embodiment alkyl is C₁₋₃-alkyl), haloalkyl, —NR⁸R^{8a}, —NR⁸C(O)OR⁹, or cycloalkyl; R⁸ is hydrogen; R^{8a} is hydrogen, alkyl (in another embodiment alkyl is C₁₋₃-alkyl), or haloalkyl; R⁹ is hydrogen or alkyl (in another embodiment alkyl is C₁₋₃alkyl).

### Embodiments Z5

In another enibodiment, the Compound is that where R¹ is pyridin-3-yl optionally substituted with one or iwo R⁷; and R⁷ is as defined in ihe Summary of the Invention for a Compound of Formula I or as defined in embodiment (1). In another embodiment, the Compound is that where R¹ is pyridin-3-yl optionally substituted with one or two R⁷; each R⁷, when present, is independently hydrogen, halo, cyano, hydroxy, alkoxy, alkyl, —NR⁸R^{8a}, —NR⁸S(O)₂R^{8a}, —S(O)R¹³, —S(O)₂R^{13a}, or —S(O)₂NR⁸R⁹ and all other groups are independently as the fined in the Summary of the Invention for a Compound of Formula I or as defined in embodiment (1). In another embodiment, the Compound is that where R¹ is pyridin-3-yl optionally substituted with two R⁷; one R⁷ is hydrrogen, halo, cyano, alkoxy, alkyl (in another embodiment alkyl is C₁₋₃-alkyl), or —NR⁸R^{8a} and the other R⁷ is —NR⁸S(O)₂R^{8a}; or one R⁷ is hydroxy or —NR⁸R^{8a} and the other R⁷ is —S(O)R¹³, —S(O)₂R^{13a}, —S(O)₂NR^{R}⁹; and all other groups are independently as defined in the Summary of the Invention for a Compound of Formula I or as defined in embodiment (1). In another embodiment, the Compound is that where R¹ is pyridin-3-yl optionally substituted with two R⁷; one R⁷ is hydrogen, halo, cyano, alkoxy, alkyl (in another embodiment alkyl is C₁₋₃-alkyl), or —NR⁸R^{8a} and the other, R⁷ is —NR⁸S(O)₂R^{8a}; or one R⁷ is hydroxy or —NR⁸R^{8a} and the other R⁷ is —S(O)R¹³, —S(O)₂R^{13a}, —S(O)₂NR⁸R⁹ ; R¹³ is hydroxyalkyl; Rl₃ₐ is alkyl or heterocycloalkyl optionally substituted with one group which is amino, alkyl, hydroxyalkyl, or hydroxy: each R⁸ and R^{8a} are independently hydrogen or alkyl; R⁹ is hydrogen, haloalkyl, alkoxyalkyl, hydroxyalkyl, aminoalkyl, alkylaminoalkyl, dialkylaminoalkyl, cycloalkyl, heterocycloalkyl, heterocycloalkylalkyl, alkyl substituted with one aminocarbonyl, or hydroxyalkyl which is substituted with one amino or 3 halo: and all otger groups are independently as defined in ihe Summary of the Invention for a Compound of Formula I or as defined in embodiment (1).

### Embodimenis (X)

In another embodiment, the Compound is that where R⁶ is —S(O)₂R⁸, —C(O)NR⁸R^{8a} or heteroaryl optionally substituted with 1,2, or 3 R¹⁴; and R⁸, R^{8a}, and R¹⁴ are independently as defined in the Summary of the Invenlion for a Compound of Formula I or as defined in embodiment (1). In another embodiment, the Compound is that where R⁶ is located in the para position of the phenyl ring to which it is attached; R⁶ is —C(O)NR⁸R^{8a} or heteroaryl optionally substituted with 1,2, or 3 R¹⁴; and R⁸, R^{8a}, and R¹⁴ are independently as defined in the Summary of the Invention for a Compound of Formula I or as defined in embodiment (1). In another embodiment, the Compound is that where R⁶ is located in the para position of the phenyl ring to which it is atiached: R⁶ is —C(O)NR⁸R^{8a} or heteroaryl optionally subsiituled with 1,2, or 3 R¹⁴: R⁸ is hydrogen, R^{8a} is hydrogen, alkyl (in another embodiment alkyl is C₁₋₃-alkyl), haloalkyl, or optionally substituted heterocycloalkyl: R¹ is alkyl (in another embodiment alkyl is C₁₋₃alkyl) or alkoxycarbonyl. In another embodiment, the Compound is that where R⁶ is located in the para position of the phenyl ring to which it is attached; R⁶ is —C(O)NR⁸R^{8a} , imidazolyl, or pyrazolyl where the imidazolyl and pyrazolyl are optionally substituted with 1,2, or 3 R¹⁴: R⁸ is hydrogen; R^{8a} is hydrogen, alkyl (in another embodiment alkyl is C₁₋₃-alkyl), haloalkyl, or optionally substituted pyrrolidinyl: R¹⁴ is alkyl (in another embodiment alkyl is C₁₋₃-alkyl) or alkoxycarbonyl. In another embodiment, the Compound is that where R⁶ is located in the meta position of the phenyl ring to which it is attached; R⁶ is —S(O)₂R⁸; and R⁸ is as defined in the Summary of the Invention for a Compound of Formula for as defined in embodiment (1). In another embodiment, the Compound is that where R⁶ is located in the meta position of the phenyl ring to which it is attached; R⁶ is —S(O)₂R⁸; R⁸ is alkyl.

### Embodiments (J)

In another embodiment, the Compound is according to Formula I(b)

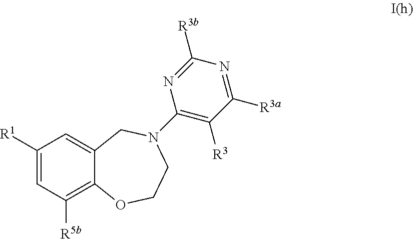

where R¹, R³,R^{3a}, and R^{3b} are independently as defined in the Summary of the Invention for a Compound of Formula I or as defined in embodiment (1). In another embodiment, the Compound of Formula I(h) is that where R³, R^{3a}, and R^{3b} are as described in any of embodiments (D3a)-(D3c), (D3g), and (D3i); and all other groups are as defined in the Summary of the Inveniion for a Compound of Formula I or as defined in embodiment (1).

In another embodiment of embodiments (J), the Compound of Formula I(h) is that where R¹ is according to any of embodiments (Z)-(Z5): and all other groups are as defined in the Summary of the Invention for a Compound of Formula I or as defined in embodiment (1).

In anolher embodiment of embodiments (K), the Compound of Formula I is according to Formula I(j):

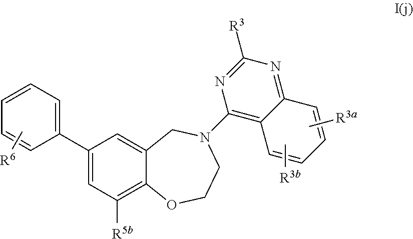

where R³, R^{3a}, R^{3b}, and R⁶ are independently as defined in the Summary of the Invention for a Compound of Formula I or as defined in embodiinenl (1). In another embodiment, the Compound is of Formula I(j) where R³, R^{3a}, and R^{3b} are as defined in embodimenls (E2b); and all other groups are as defined in the Summary of the Invention for a Compound of Formula I or as defined in embodiment (1). In another embodiment, the Compound is of Formula I(j) where R³ is hydrogen, alkyl (in another embodiment alkyl is C₁₋₃-alkyl), halo, —OR^{11a}, or alkyl substituted with one R¹⁶; R³ is hydrogen: R^{3a} is hydrogen or alkoxy: and R⁶ is as defined in the Summary of the Invention for a Compoundd of Forinula I or as defined in embodiment (1).

In another embodiment of embodiments (K), the Compound of Formula I(j) is that where R⁶ is according to embodiments (X); and all other groups are as defined in the Summary of the Invention for a Compound of Formula I or as defined in embodiment (1).

In another embodiment of embodiments (L). the Compound of Formula I is according to Formula I(k):

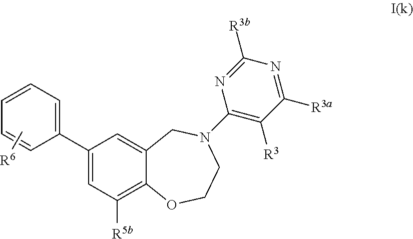

where R³, R^{3a}; R^{3b}, and R⁶ are independently as defined in the Summary of the Invention for a Compound of Formula I or as defined in embodiment (1). In another embodiment, the Compound of Formula I(h) is that where R³ , R^{3a}, and R^{3b} areas described in any of embodiments (D3a)-(D3c), (D3g), and (D3i); and all other groups are as defined in the Summary of the Inveniton for a Compound of Formula I or as defined in embodiment (1).

In another embodiment of embodiments (L), the Compound of Formula I(k) is that where R⁶ is according lo embodimenis (X); and all other groups are as defined in the Summary of ihe Invention for a Compound of Formula I or as defined in embodiment (1).

In another embodiment of embodiments (M), the Compound of Formula I is accordinu to Formula I(m):

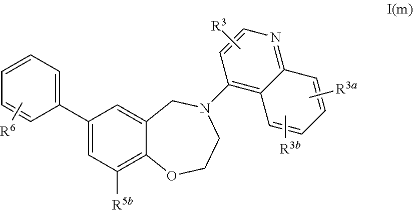

where R³, R^{3a}, R^{3b}, and R⁶ are indepehdcnllyvasindependently as defined in the Summary, of the Invention for a Compound of Formula I or as defined in embodiment (I). In another embodiment, the Compound is of Formula I(m) where R³ is hydrogen, alkyl (in another embodiment alkyl is C₁₋₃alkyl), or alkyl substituted with one R¹⁶, —OR^{11a}; R^{3a} is hydrogen or —OR^{11a}; and R^{3b} is hydrogen or alkyl; and R⁶ is as defined in the Summary of ihe Invention for a Compound of Formula I or as defined in embodiment (1). In another embodiment, the Compound is of Formula I(m) where R³, R^{3a}, and R^{3b} are as defined in embodimenis (E6a): and R⁶ is as defined in the Summary of the Invention for a Compound of Formula I or as defined in embodimeni (1).

In another embodiment of embodiments (M), the Compound of Formula I(m) is that where R⁶ is according to embodiments (X); and all other groups are as defined in the Summary of ihe Invention for a Compound of Formula I or as defined in embodiment (1).

In another embodiment of embodiments (N), the Compound is of Formula I(n):

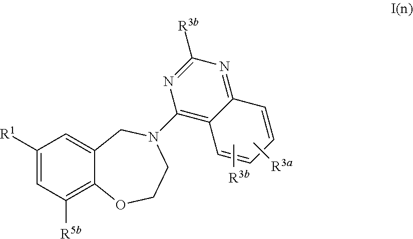

where R¹ is as defined in the Summary of the Invention for a Compound of Formula I or as defined in embodiment (1); and one of R³, R^{3a}, and R^{3b} and all other groups are independently as defined in the Summary of the Invention for a Compound of Formula I or as defined in embodiment (1). In another embodiment of embodiments (N). the Compound is of Formula I(n) where R³,R^{3a}, R^{3b}, and R¹ are independently as defined in the Summary of the Invention for a Compound of Formula I or as defined in embodiment (1). In another embodiment, the Compound is of Formula I(n) where R³, R^{3a} , and R^{3b} is as defined in embodiments (E2b); and all other groups, are as defined in the Summary of the Invention for a Compound of Formula I or as defined in embodiment (1). In another embodiment, the Compound is of Formula I(n) where R³ is hydrogen, alkyl, (in another embodiment alkyl is C₁₋₃-alkyl), halo, —OR^{11a}, or alkyl substituted with one R¹⁶; R³ is hydrogen; R^{3a} is hydrogen or alkoxy; and R¹ is as defined in the Summary of the Invention for a Compound of Formula I or as defined in embodiment (1). In another embodiment, the Compound is of Formula I(a) where R¹ is as defined in the Summary of the Invention for a Compound of Formula I or as defined in embodiment (1); and two of R³, R^{3a}, and R^{3b} are hydrogen and the others are independently as defined in the Summary of the Invention for a Compound of Formula I or as defined in embodiment (1). In another embodiment, the Compound is of Formula I(n) where R¹ is as defined in the Summary of the Invention for a Compound of Formula I or as defined in embodiment (I); and three of R³, R^{3a}, and R^{3b} are hydrogen and the others are independently as defined in the Summary of the Invention for a Compound of Formula I or as defined in embodiment (1).

In another embodiment of embodiments (N), the Compound of Formula I(n) is that where R¹ is according to any of embodiments (Z5); and all other groups areas defined in the Summary of the Invention for a Compound of Formula I or as defined in embodiment (I).

### Embodiments (P)

In another embodiment, the Compound is of Formula I(p):

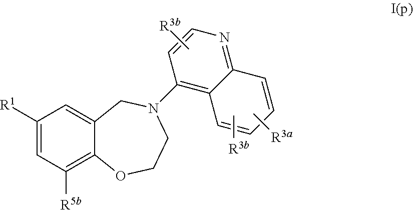

where R¹ is as defined in the Summary of the Invention for a Compound of Formula I; and one of R³, R^{3a}, and R^{3b} is hydrogen and the others are independently as defined in the Summary of the Invention for a Compound of Formula I. In another embodiment, the Compound is of Formula I(p) where R¹ is as defined in the Summary of the Invention for a Compound of Formula I; and one of R³, R^{3a}, and R^{3b} are hydrogen and the others are independently as defined in the Summary of the Invention for a Compound of Formula I. In another embodiment, the Compound is of Formula I(p) where R¹ is as defined in the Summary of the Invention for a Compound of Formula I; and two of R³, R^{3a} , and R^{3b} are hydrogen and the others are independently as defined in the Summary of the Invention for a Compound of Formula I. In another embodiment, the Compound is of formula I(p) where R³ is hydrogen, alkyl (in another embodiment alkyl is C₁₋₃alkyl), or alkyl substituted with one R¹⁶, —OR^{11a}; R^{3a} is hydrogen or —OR^{11a}; and R^{3b} is hydrogen or alkyl; and R⁶ is as defined in the Summary of the Invention for a Compound of Formula I or as defined in embodiment (1). In another embodiment, the Compound is of Formula I(p) where R³, R^{3a}, and R^{3b} are as defined.in embodiments (E6a); and R⁶ is as defined in the Summary of the Invention for a Compound of Formula I or as defined in embodiment (1).

In another emnbodiment of embodiments (P), the. Compound of Formula I(p) is that where R¹ is according to any of embodiments (Z)-(Z5): and all other groups are as defined in the Summary of the Invention for a Compound of Formula I or as defined in embodiment (1).

### Embodiments O

In another embodiment, the Compound is of Formula I(q):

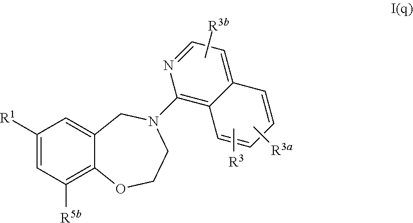

where R¹ is as defined in the Summary of the Invention for a Compound of Formula I: and one of R³, R^{3a}, and R^{3b} is hydrogen and the others are independently as defined in the Summary of the Invention for a Compound of Formula I. In another embodiment, the Compound is of Formula I(q) where R¹ is as defined in the Summary of the Invention for a Compound of Formula I; and two of R³, R^{3a}, and R^{3b} are hydrogen and the others are independently as defined in the Summary of the Invention for a Compound of Formula I. In another embodiment, the Compound is of Formula I(q) where R¹ is as defined in the Summary of the Invention for a Compound of Formula I: and three of R³, R^{3a}, and R^{3b} are hydrogen and the others are independently as defined in the Summary of the Invention for a Compound of Formula I.

In another embodiment of embodiments (Q), the Compound of Formula I(q) is that where R¹ is according to any of embodiments (Z)-(Z5); and all other groups are as defined in the Summary of the Invention for a Compound of Formula I or as defined in embodiment (I).

### Embodiment (F)

In another embodiment, the Compound is of Formula I(r):

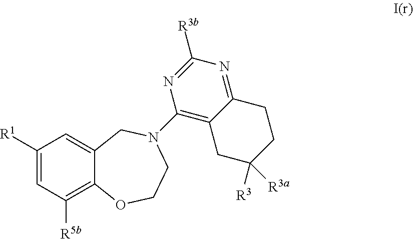

where R¹, R³, R^{3a}, and R^{3b} are independently as defined in the Summary of the Invention for a Compound of Formula I. In another embodiment, the Compound of Formula I(r) is where R³ and R^{3a} are alkyl (in another embodiment alkyl is C₁₋₃-alkyl) and R^{3b} is hydrogen, alkyl (in another embodiment alky is C₁₋₃-alkyl), haloalkyl, or alkyl substituted with one R¹⁶;and all other groups are as defined in the Summary of the Invention for a Compound of Formula I or as defined in embodiments (1). In another embodiment, the Compound of Formula I(r) is where R³ and R^{3a} are halo and R^{3b} is hydrogen, alkyl (in another embodiment alkyl is C₁₋₃-alkyl), haloalkyl, or alkyl substituted with one R¹⁶; and all other groups are as defined in the Summary of the Invention for a Compound of Formula I or as defined in embodiment (1). In another embodiment, the Compound of FormulaI(r) is where R³ and R^{3a} together with the carbon to which they are attached form an optionally substituted cycloalkyl and R^{3b} is hydrogen, alkyl (in another embodiment alkyl is C₁₋₃-alkyl), haloalkyl, or alkyl substituted with one R¹⁶; and all other groups are as defined in the Summary of the Invention for a Compound of Formula I or as defined in embodiment (1).

In another embodiment of embodimenls (F), the Compound of Formula I(r) is that where R¹ is according lo any of embodiments (Z)-(Z5); and all other groups are as defined in the Summary of the Invention for a Compound of Formula I or as defined in embodiment (1).

### Embodiments (S)

In another embodiment, the Compound is of FormulaI(s):

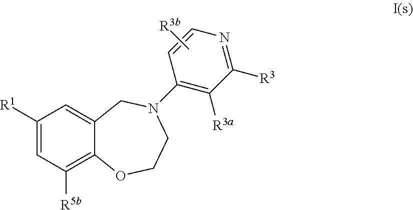

where R³ is cyano, alkyl (in another emebodiment alkyl is C₁₋₃-alkyl), halo, haloalkyl, —SR¹¹, alkylsulfonyl, optionally substituted phenyl, optionally substituted phenylalkyl, optionally substituted cycloalkyl, optionally substituted cycloalkylalkyl, carboxy. —C(O)OR¹, —NR¹¹R^{11a}, or —OR^{11a}; and R¹, R^{3a}, R^{3b}, R^{3c}, R¹¹, and R^{11a} are independently as defined in the Summary of the Invention for a Compound of Formula I.

In another embodiment of embodiments (S), the Compound of Formula I(s) is that where R¹ is according to any of embodiments (Z)-(Z5); and all other groups are as defined in the Summary of the Invention for a Compound of Formula I or as defined in embodiment (1).

### Embodiments (T)

In another embodiment, the Compound is of Formula I(f):

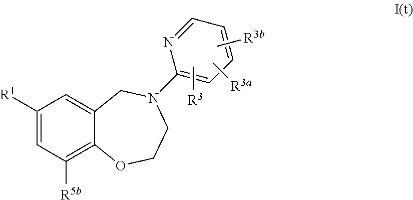

where R¹, R³, R^{3a}, and R^{3b} are independently as defined in the Summary of the Invention for a Compound of Formula I.

In another embodimeni of embodimenis (T), the Compound of Formula I(t) is that where R¹ is according to any of embodiments (Z)-(Z5); and all other groups are as defined in the Summary of the Invention for a Compound of Formula I or as defined in embodiment (1).

### Embodiment (U)

In another embodiment, the Compound is according to Formula I (a) where R¹ is heteroaryl optionally substituted with one or two R⁷; each R⁷n when present, is independently halo, alkyl, cycloalkyl, haloalkyl, hydroxyalkyl, alkoxyalkyl, —NR⁸R^{8a}, or —NR⁸C(O)OR⁹; and all ovher groups areindependently, as defined inthe Summary of the Invention for a Compound of Formula I. In another embodiment, the Compound is according to Formula I(a)where R¹ is heteroaryl optionally substituted with one or two R⁷; each R⁷. when present, is independently alkyl (in another embodimeni alkyl is C₁₋₃-alkyl), cycloalkyl, haloalkyl, —NR⁸R^{8a}, or —NR⁸C(O)OR⁹; and all other groups are independently as defined in the Summary of the Invenlion for a Compound of Formula I. In another embodiment, the Compound is according lo Formula I(a) where R¹ is heteroaryl optionally substituted with one or two R⁷; each R⁷, when present, is independently alkyl (in another embodiment alkyl is C₁₋₃-alkyl), cycloalkyl, haloalkyl, —NR⁸R^{8a} or —NR⁸C(O)OR⁹: R⁸ is hydrogen; R^{8a} is hydrogen, alkyl (in another embodiment alkyl is C₁₋₃-alkyl), or haloalkyl: and R⁹ is hydrogen or alkyl (in another embodiment alkyl is C₁₋₃-alkyl); and all other groups are independently as defined in Ihe Summary of the Invention for a Compound of Formula I.

In another embodiment, the Compound is according lo Formula I(a) where R² is 5,6,7,8-tetrahydrpoquinolin-4-yl or 5,6,7,8-tetrahydroisoquinolin-1-yl, where R² is substituted with R³, R^{3a}, R^{3b}, R^{3c}, and R^{3d}, an R¹, R³, R^{3a}, R^{3b}, R^{3c}, and R^{3d} are independently as defined in the Summary of the Invention for a Compound of Formula I. In another embodiment, the Compound is according to Formula I(a) where R² is 5,6,7,8-tetrahydroquinolin-4-yl or 5,,7,8-tetrahydroisoquinolin-1-yl, where R² is substituted wilh R³, R^{a3}, R^{3b}, R^{3c}, and R^{3d}; R^{3d} is hydrogen; and R¹, R³, R^{3a}, R^{3b}, and R^{3c} are independently as defined in the Summary of the Invention for a Compound of Formula I. In another embodiment, the Compound is according to Formula I(a) where R² is 5,6,7,8-tetrahydroquinolin-4-yl or 5,6,7,8-tetrahydroisoqinolin- 1-yl, where R² is substituted with R³, R^{3a} , R^{3b}, R^{3c}, and R^{3d}, R^{3b}, R^{3c}, and R^{3d} are hydrogen: and R¹, R³, and R^{3a} are independently as defined in the Summary of the Invention for a Comppound of Formula I. In another embodiment, the Compound is according to Formula I(a) where R² is 5,6,7,8-tetradroquinolin-4-yl or 5,6,7,8-tetrahydroisoquinolin-1-yl, where R² is substituted with R³, R^{3a}, R^{3b}, R^{3c}, and R^{3d}, R^{3a}, R^{3b}, R^{3c} and R^{3d} are hydrogen: and R¹, and R³ are indepehdenily as defined in the Summary of the Invention for a Compound of Formula I. In another embodiment, the Compound is according to Formula I(a) where R² is 5,6,7,8- tetrahydroquinolin-4-yl or 5,6,7,8-tetrahydroisoquinolin-1-yl, where R² is substituted with R³, R^{3a}, R^{3b}, R^{3c}, and R^{3d}, R³, R^{3a}, R^{3b}, R^{3c}, and R^{3d} are hydrogen: and R¹ is as defined in the Summary of the Invention for a Compound of Formula I

In another embodiment, R² in the compound of formula I is optionally substituted

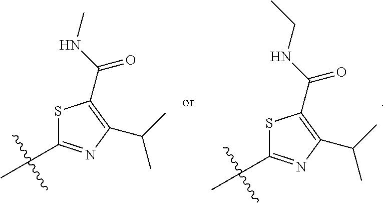 [SMILES: *c1nc(C(C)C)c(C(=O)NC)s1 ; *c1nc(C(C)C)c(C(=O)NCC)s1]

thiazolyl. In some embodiments, R² is

In another embodiment, R² is an optionally substituted dihydrothiazolol 5,4- c]pyridin-4(5H)-one, or an optionally substituted dihydrobenzo[d]thiazol-7(4H)-one.

In another embodiment, R² in the compound of formula I is optionally substituted

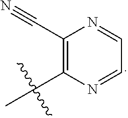 [SMILES: *c1nccnc1C#N]

pyrazinyl. In some embodiments, R² is

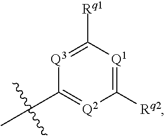

In another embodiment, R² in the compound of formula I is wherein

R¹ is H, (C₁-C₆)alkyl, (C₁-C₆)alkylene-OH, (C₁-C₆)alkyl3ne-O(C₁-C₆)alkyl, (C₁-C₆)alkylene-NH₂, (C₁-C₆)alkylene-NH(C₁-C₆)alkyl, (C₁-C₆)alkylelene-N(C₁-C₆)alkyl)₂, (C₁-C₆)alkylene-NHSO₂-(C₁-C₆)alkyl, (C₁-C₆)alkylene-NH(C═O)-(C₁-C₆)alkyl, NH₂, NH(C₁- C₆)alkyl, N((C₁-C₆)alkyl₂, (C₁-C₆)alkylene-NHSO₂-(C₁-C₆)alkyl, (C₁-C₆)alkylene- NH(C═O)-(C₁-C₆)alkyl, -(C═O)-NH₂, -(C═O)-NH(C₁-C₆)alkyl, -(C═O)-NH (C₁-C₆)alkyl))₂, NHSO₂-(C₁-C₆)alkyl, -CN, or (C₁-C_{6)alkylene-(C}₃-C₇)hetereocyclo;

R^{q2} is H, (C₁-C₆)alkyl, (C₁-C₆)alkenyl, halo, halo(C₁-C₆)alkyl, NH₂, NH(C₁-C₆)alkyl,

Q¹is N, C—H, or C-(C₁-C₆)alkyl;

Q²is N or C—R^{a}, wherein R^{a} is H, halo, (C₁-C₆)alkyl, (C₂-C₆)alkenyl, (C₁-C₆)alkylene- O(C₁-C₆)alkyl, (C₁-C₆)alkylene-OH, (C₁-C₆)alkylene-CO₂(C₁-C₆)alkyl, (C₁-C₆)alkylene- CO₂H, aryl, halo(C₁-C₆)alkyl, (C₃-C₇)cycloalkyl, (C₁-C₆)alkylene-(C₃-C₇)cycloalkyl, COH, CO₂H, CO₂(C₁-C₆)alkyl, CN, (C₁-C₆)alkylene-CN, (C₁-C₆)alkylene-C≡C—H, (C₁-C₆)alkylene- C≡C-(C₁-C₆)alkyl, —C≡H, —C≡C-(C₁-C₆)alkyl, (C₁-C₆)alkylene; or

R^{a} and R^{q2}, together with the atoms to which they are attched, can be joined together to form an substituted 5, 6, or 7 membered saturated or unsaturated ring, optionally containing up to two lieteroatoms selected from N—H, N-(C₁-C₆)alkyl, O, SO, SO₂; and

Q³ is N or C-R³, wherein R^{c} is H, halo, or (C₁-C₆)alkyl.

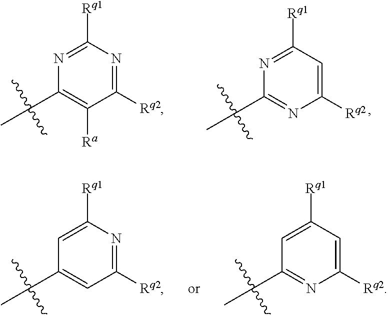

In some embodiments,

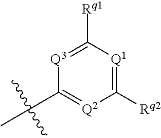

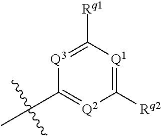

In some embodiments where

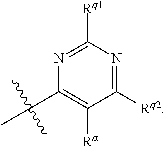

R^{q1} is NH₂, )alkyl, (C₁-C₆)alkylene-OH, (C₁C₆)alkylene-O(C₁-C₆)alkyl, (C₁-C₆)alkylene-NH₂, (C₁-C₆)-alkylene-NH(C₁-C₆)alkyl, (C₁-C₆)alkylelene-N(C₁-C₆)alkyl)₂, C₁-C₆)alkylene-NHSO₂-(C₁-C₆)alkyl, (C₁-C₆)alkylene-NH(C=O)-(C₁-C₆)alkyl, NH₂, NH(C₁- C₆)alkyl, N((C₁C₆)alkyl)₂,(C₁-₆)alkylene-NHSO₂-(C₁-C_{6)alkyl, (C}₁-C₆)alkylene- NH(C=O)-(C₁-C₆)alkyl, -(C=O)-NH₂, (C=O)-NH(C₁-C₆)alkyl, -(C=O)-NH (C₁-C₆)alkyl))₂, -NHSO₂-(C₁-C₆)alkyl, -CN, or (C₁-C₆)alkylene-(C₃-C₇)heterocyclo, wherein when any alkylene is - CH₂- , then one of the hydrogens of the -CH₂- can optionally be replaced by (C₁-C₃)haloalkyl;

R^{q2} is H, (C₁-C₆)alkyl, (C₁-C₆)alkenyl, halo, halo(C₁-C₆)alkyl, NH₂, NH(C₁-C₆)alkyl, N((C_{1-C}₆)alkyl)₂; and

R^{a} and R^{q2}, together with the atoms to which they are attached, can be joined together to form an substituted 5, 6, or 7 membered saturated or unsaturated ring, optionally containing up to two heteroatoms selected from N-H, N-(C₁-C₆)alkyl, O, SO, S₀₂.

In some embodiments,

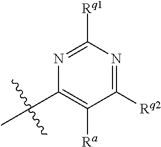

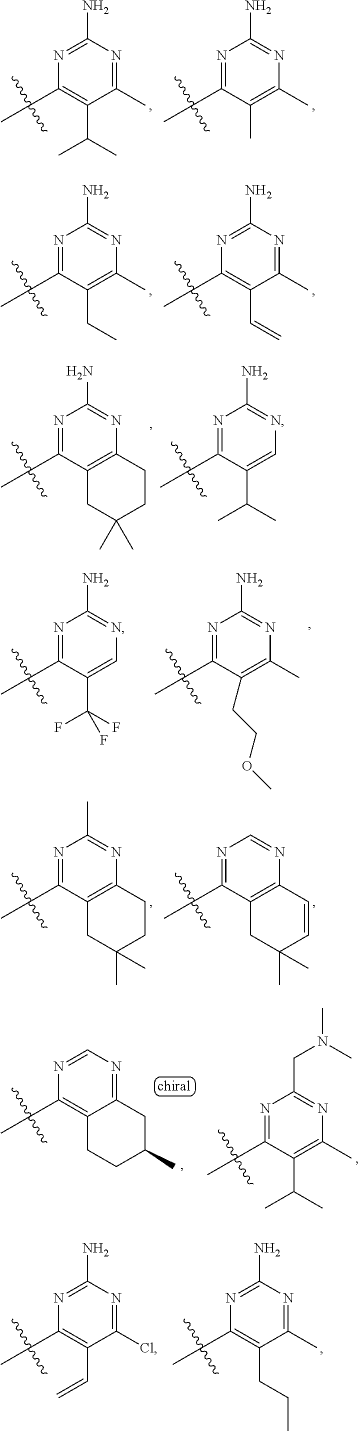

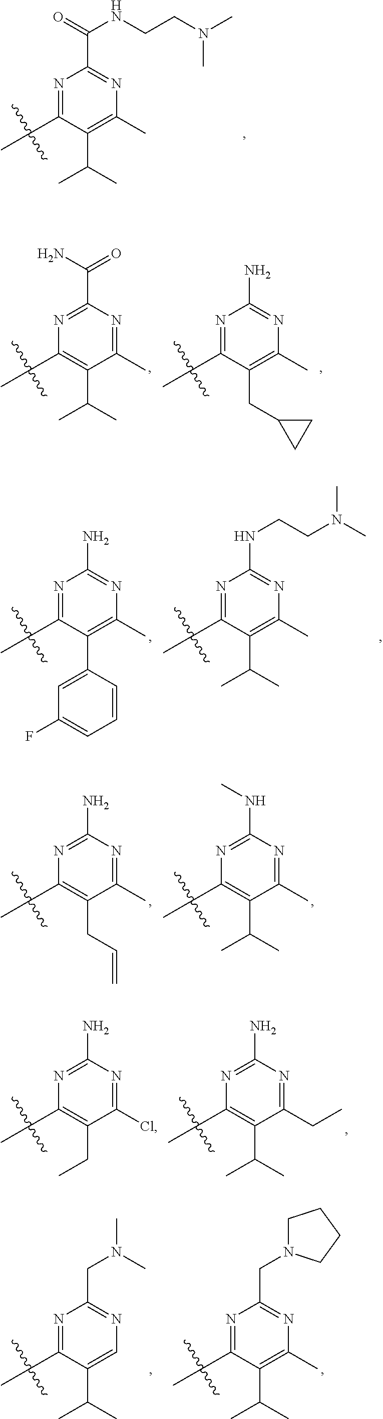

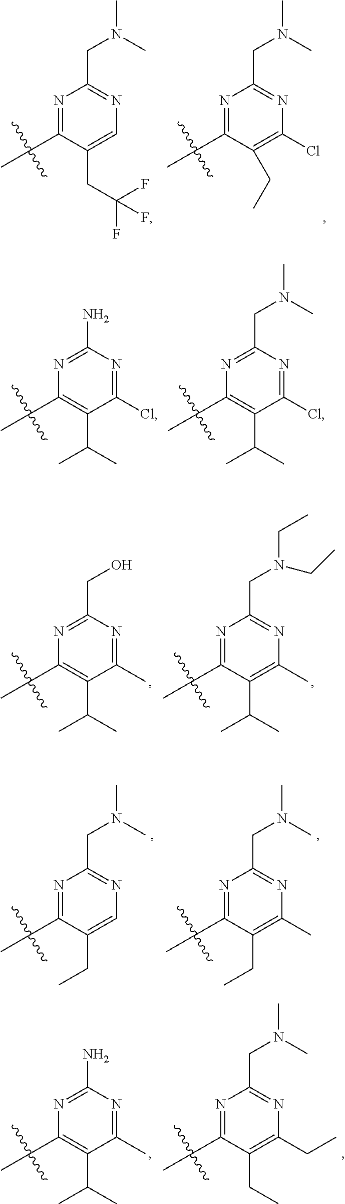

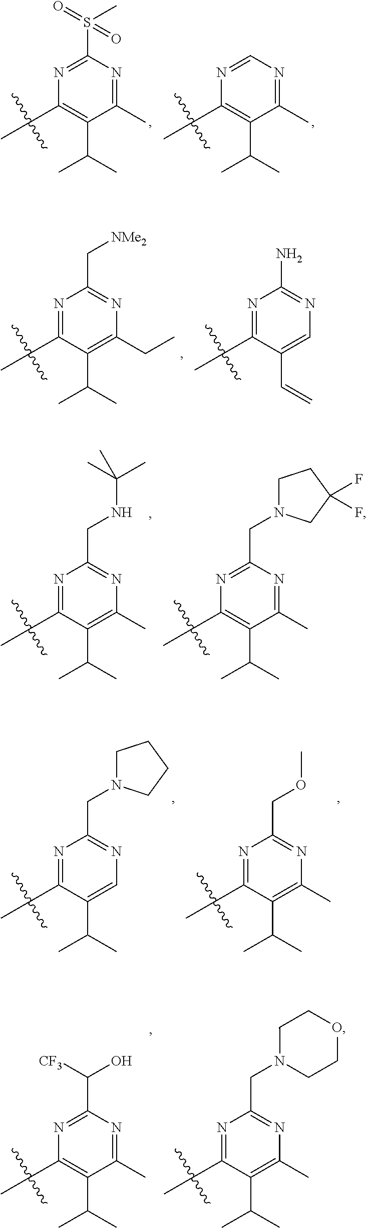

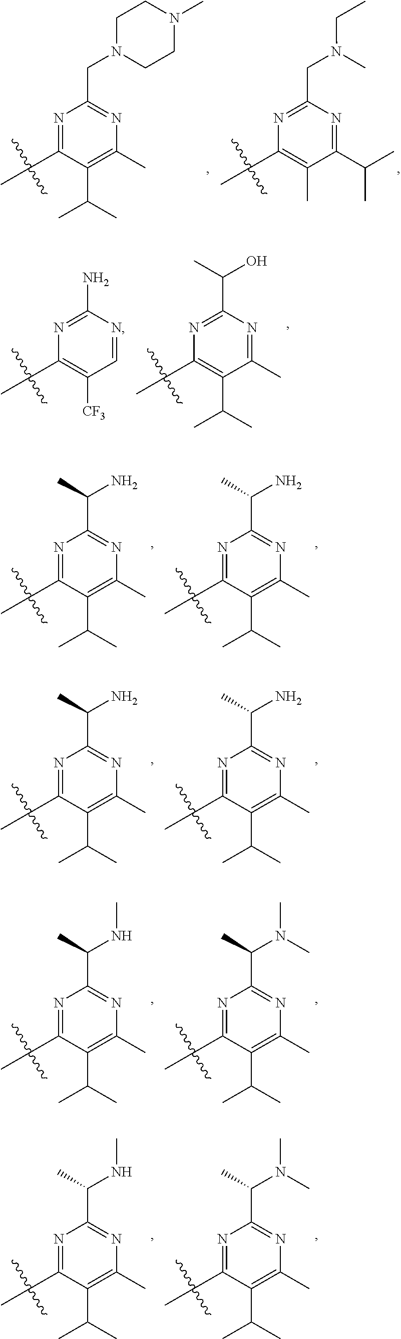

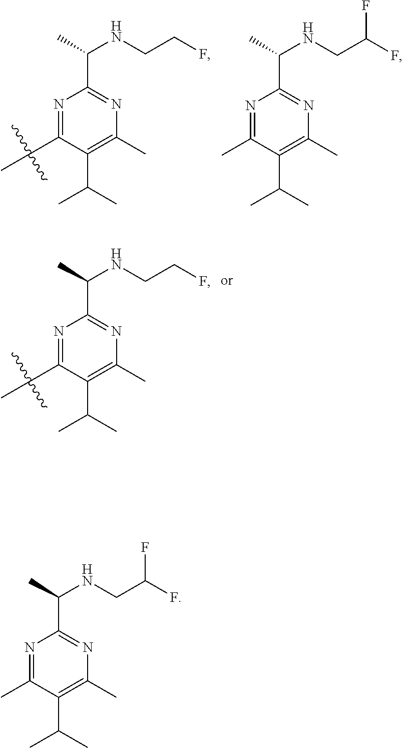 [SMILES: *c1nc([C@H](C)NCCF)nc(C)c1C(C)C ; Cc1nc([C@H](C)NCC(F)F)nc(C)c1C(C)C ; *c1nc([C@@H](C)NCCF)nc(C)c1C(C)C ; "or" ; Cc1nc([C@@H](C)NCC(F)F)nc(C)c1C(C)C]

In another embodiment,

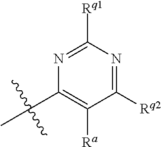

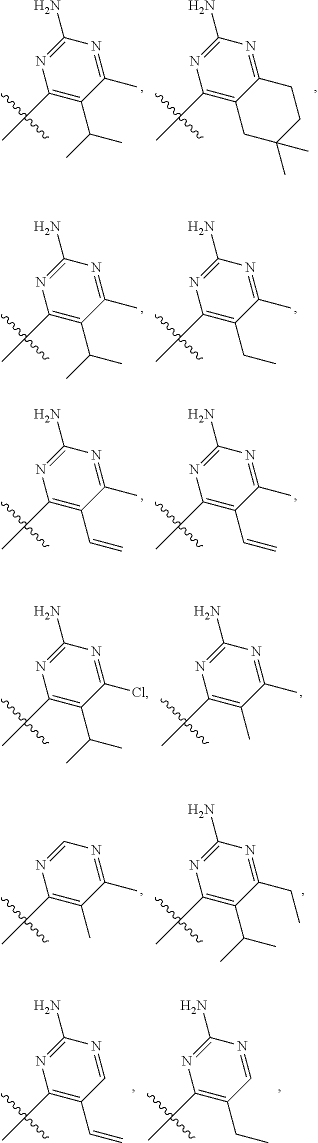

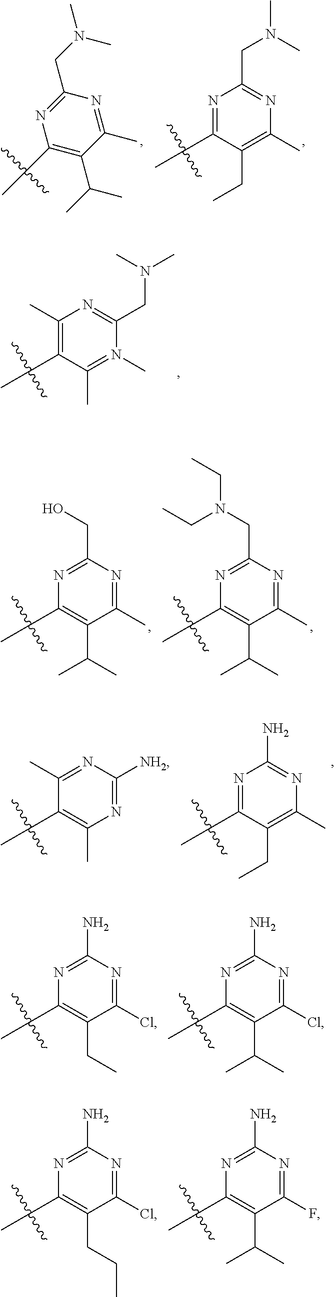

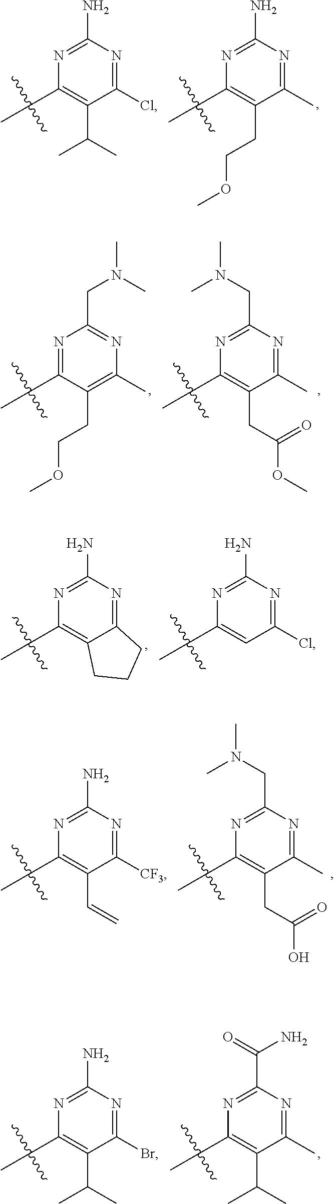

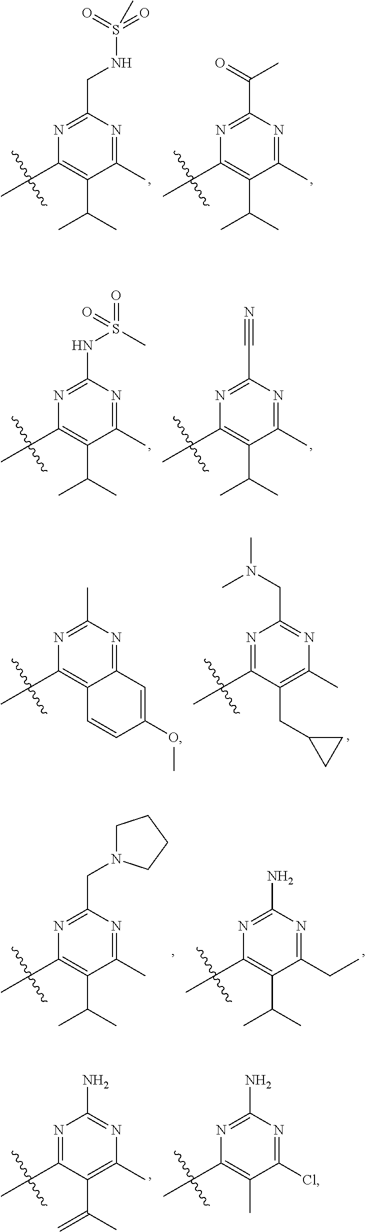

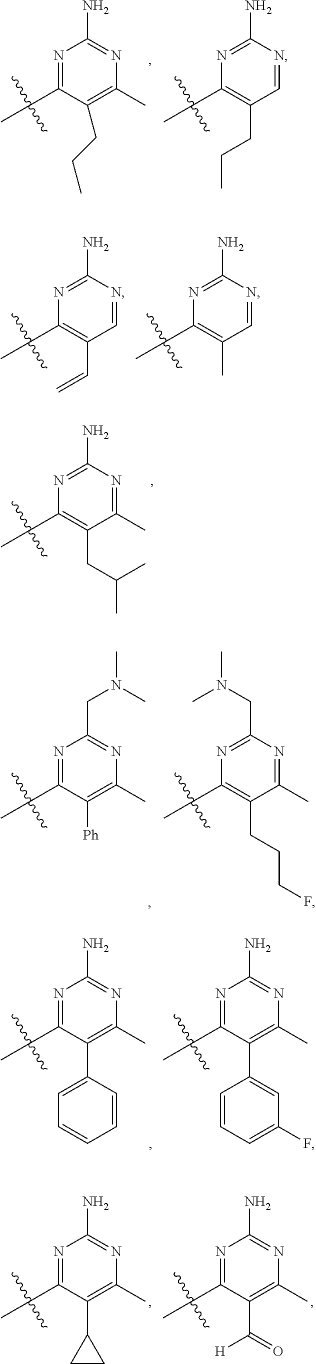

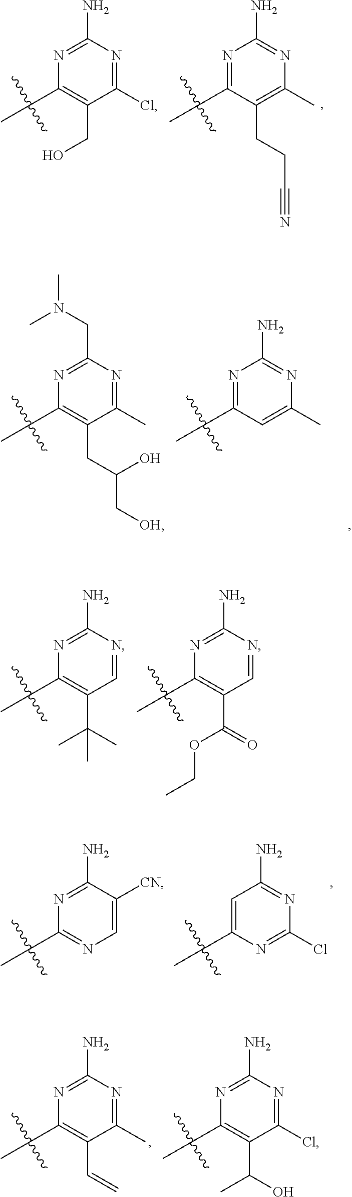

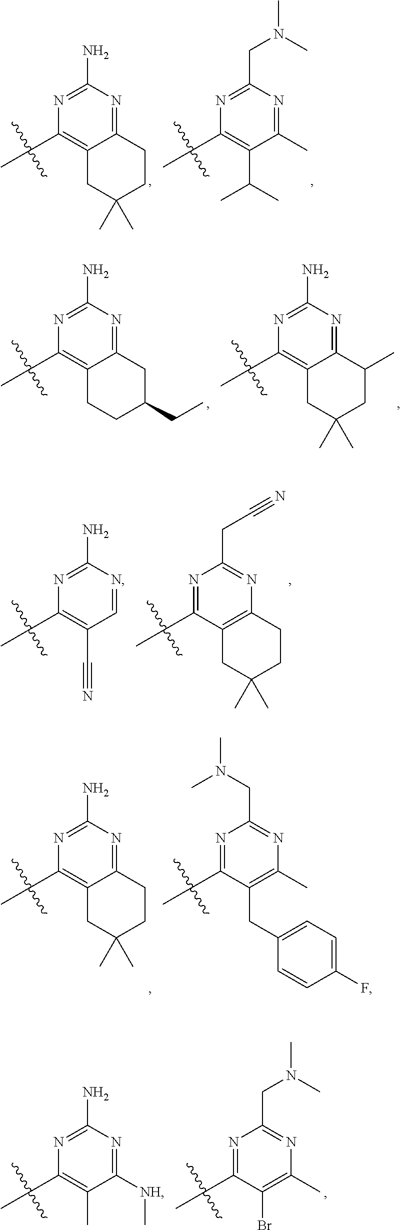

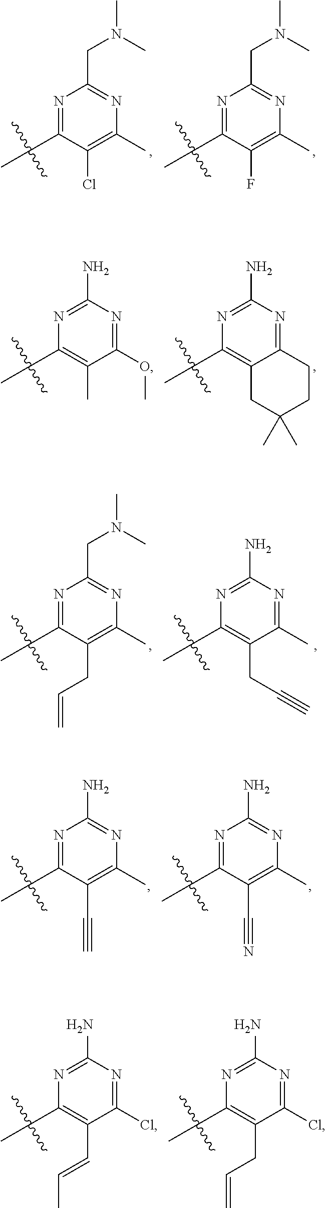

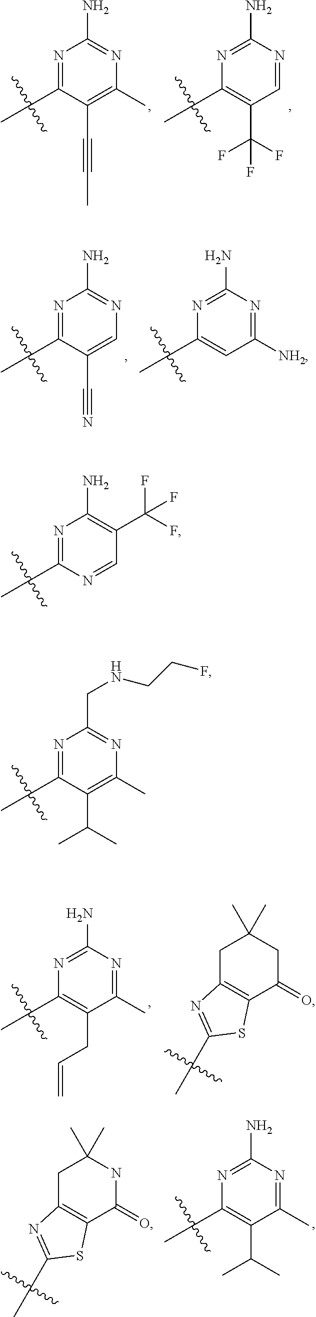

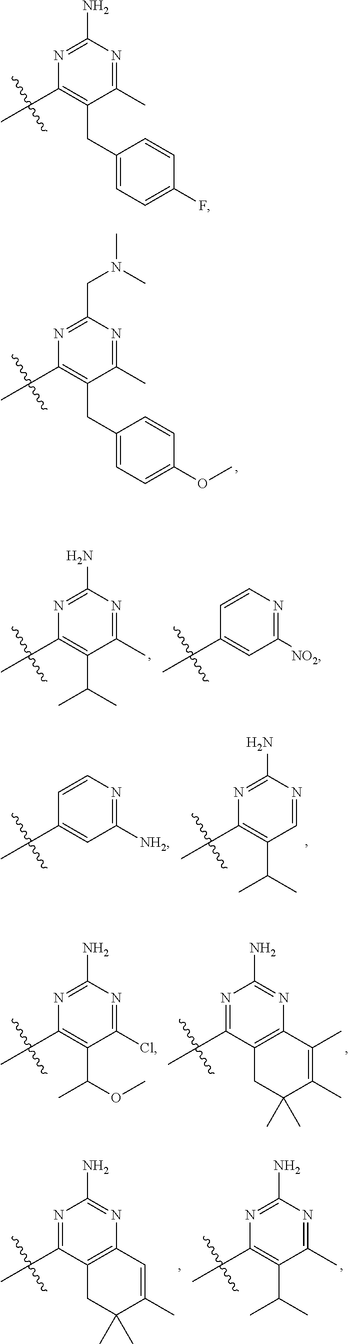

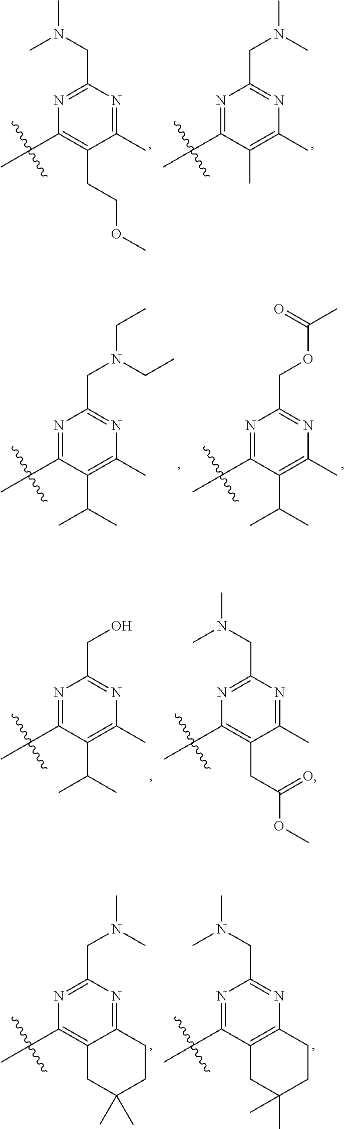

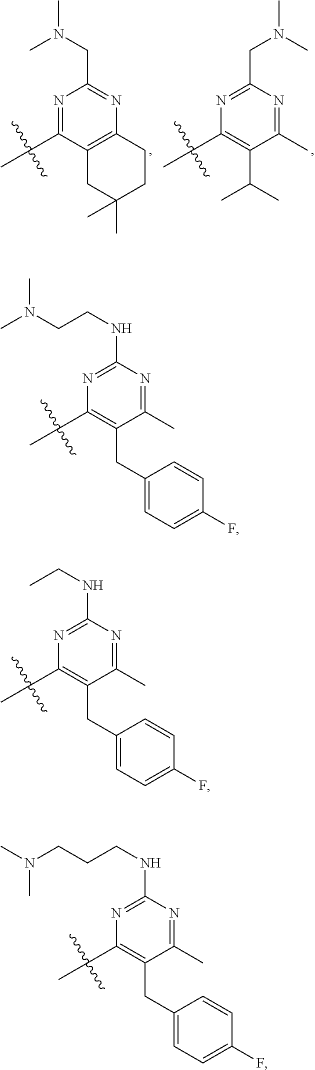 [SMILES: *c1nc(CN(C)C)nc2c1CC(C)(C)CC2 ; *c1nc(CN(C)C)nc(C)c1C(C)C ; *c1nc(NCCN(C)C)nc(C)c1Cc1ccc(F)cc1 ; *c1nc(NCC)nc(C)c1Cc1ccc(F)cc1 ; *c1nc(NCCCN(C)C)nc(C)c1Cc1ccc(F)cc1]

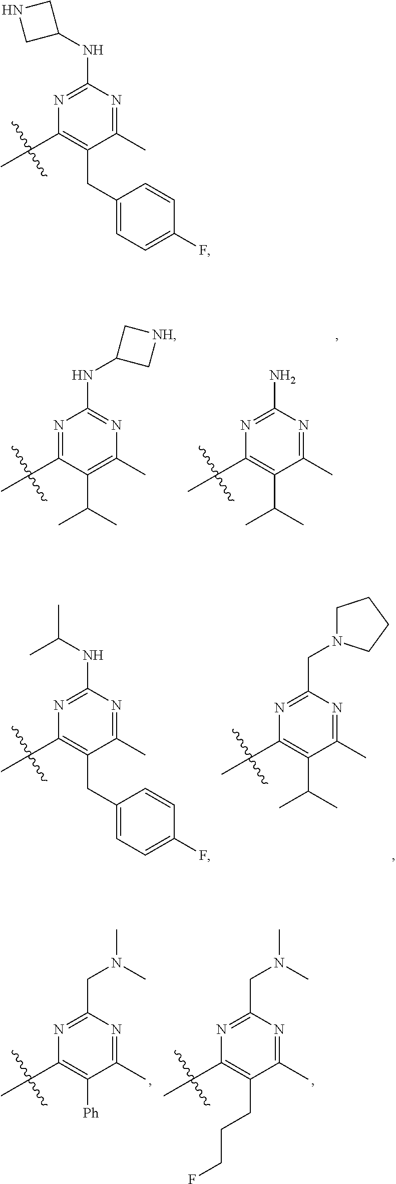

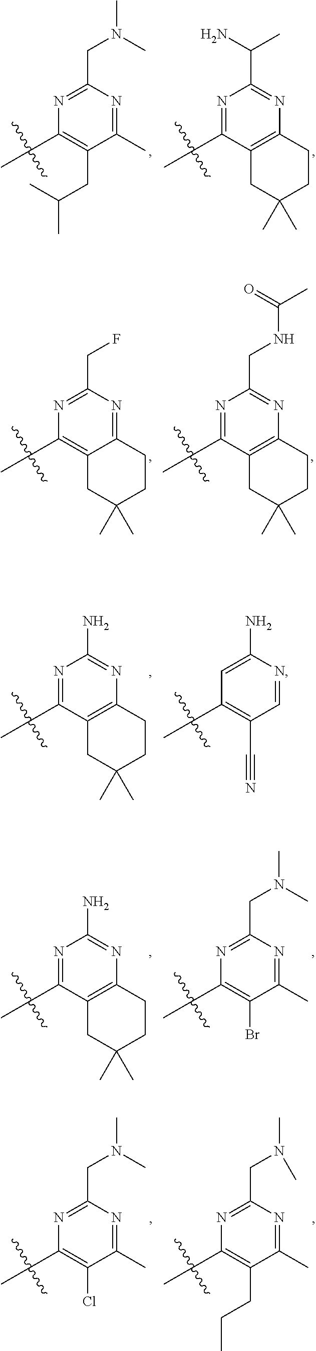

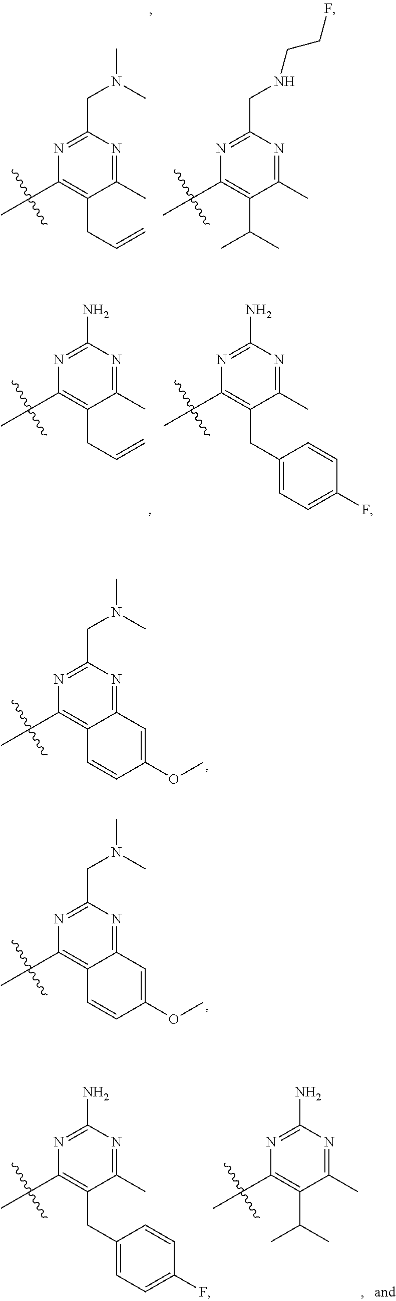

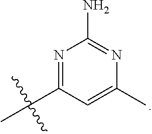 [SMILES: *c1cc(C)nc(N)n1]

In another embodiment,

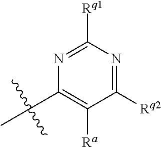

is, is

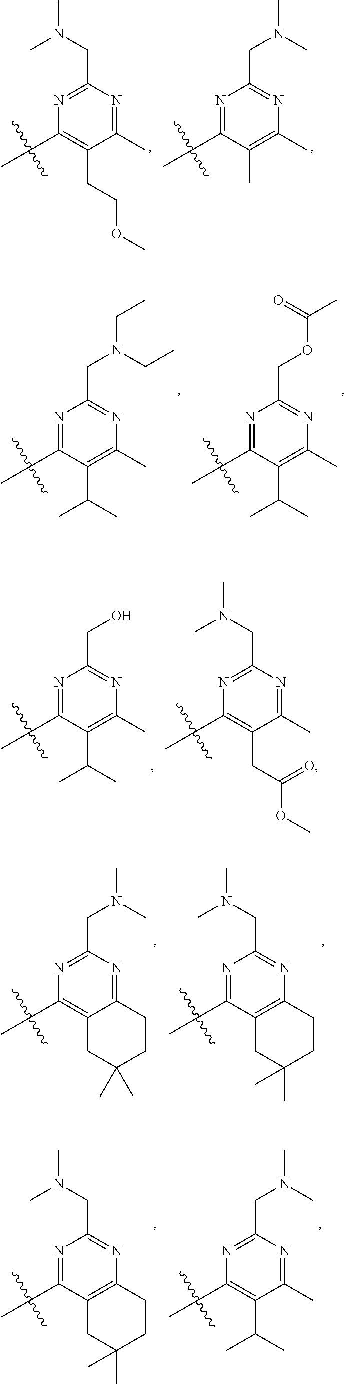

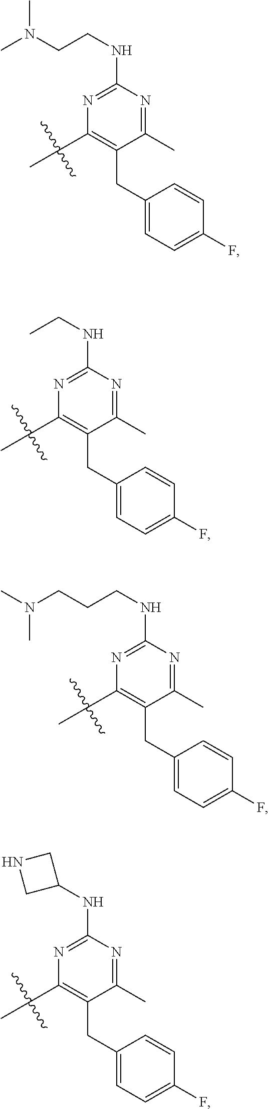 [SMILES: *c1nc(NCCN(C)C)nc(C)c1Cc1ccc(F)cc1 ; *c1nc(NCC)nc(C)c1Cc1ccc(F)cc1 ; *c1nc(NCCCN(C)C)nc(C)c1Cc1ccc(F)cc1 ; *c1nc(NC2CNC2)nc(C)c1Cc1ccc(F)cc1]

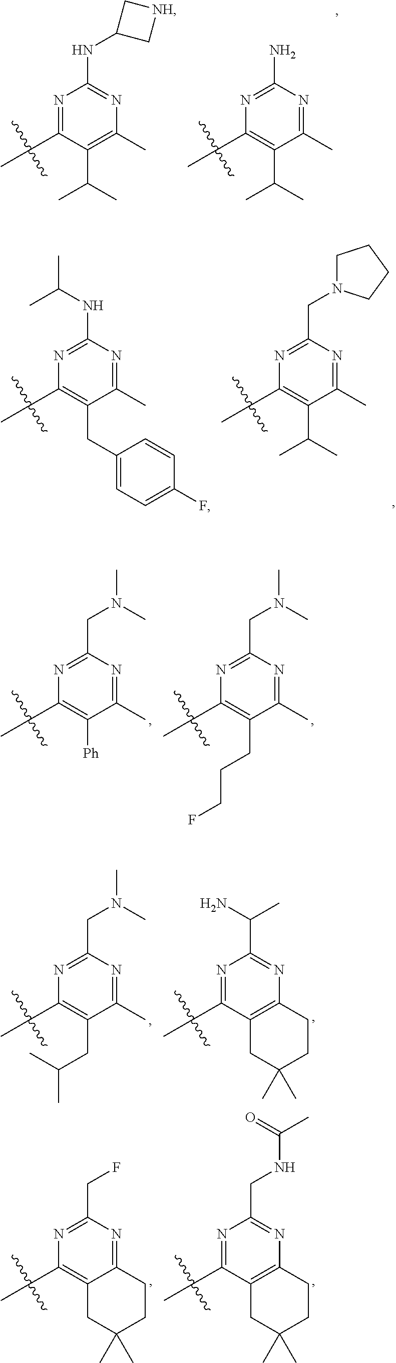

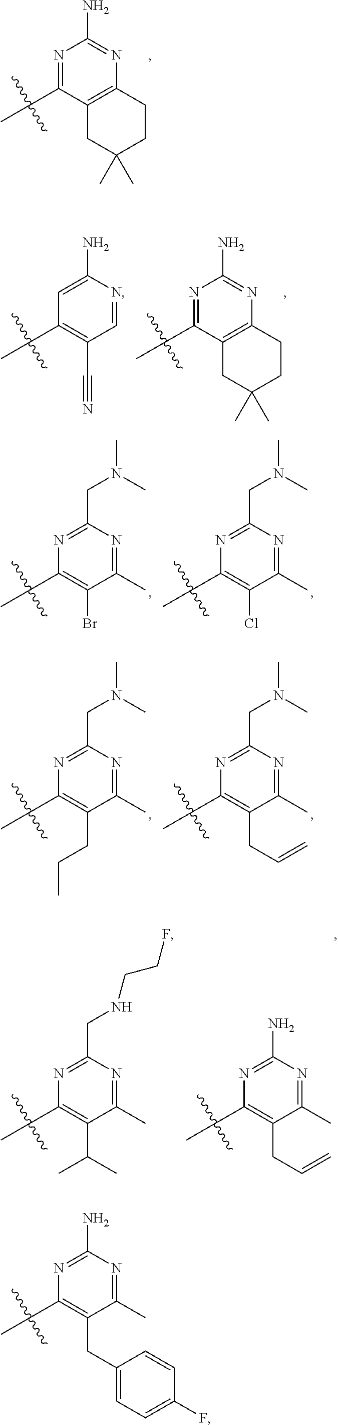

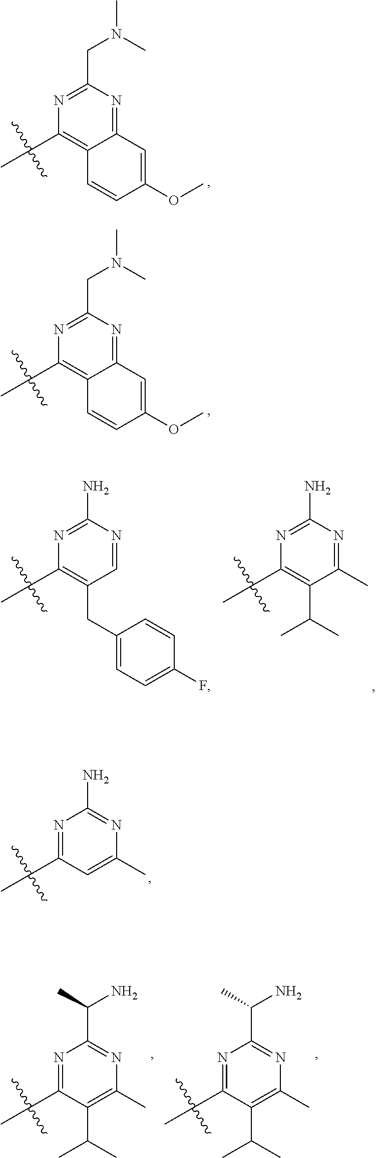

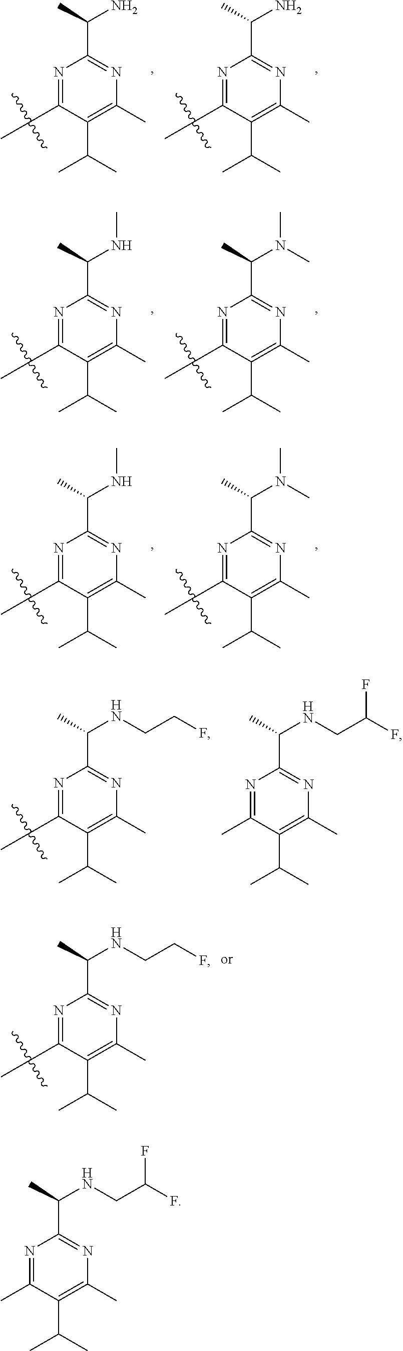

In another embodiment,

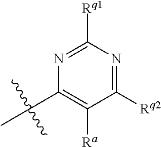

is

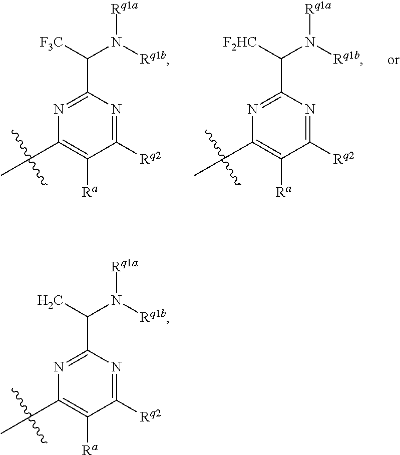

wherein R^{q1a} and R^{q1b} are each independently H, (C₁-C₆)alkyl, or halo (C₁-C₆)alkyl.

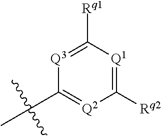

In some embodiments where

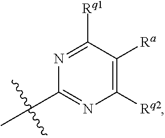

wherein R^{a} is defined as above; and

R^{q1} is H, NH₂, (C₁-C₆)alkyl, (C₁-C₆)alkylene-OH, (C₁-C₆)alkylene-O(C₁-C₆)alkyl, (C₁-C₆)alkylene-NH₂,(C₁-C₆)alkylene-NH(C₁-C₆)alkyl, (C₁-C₆)alkylelene-N(C₁-C₆)alkyl)₂, (C₁-C₆)alkylene-NHSO₂-(C₁-C₆)alkyl, (C₁-C₆)alkylene-NH(C=O)-(C₁-C₆)alkyl, NH₂, NH(C₁-C₆)alkyl, N((C₁-C₆alkyl)₂, (C₁-C₆)alkylene-NHSO₂-(C₁-C₆)alkyl, (C₁-C₆)alkyelene- NH(C_{C=O)-(C}₁-C₆)alkyl, -(C=O)-NH₂, -(C=O)-NH(C₁-C₆)alkyl, -(C=O)-NH(C₁-C₆)alkyl))₂, —NHSO₂-(C₁-C₆)alkyl, -CN, or (C₁-C₆)alkylene-(C₃-C₇)heterocyclo; and

R^{q2} is H, (C₁-C₆)alkyl, (C₁-C₆)alkenyl, halo, halo(C₁-C₆)alkyl, NH₂, NH(C₁-C₆)alkyl, N((C₁-C₆)alkyl)₂.

In some embodiments,

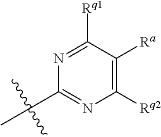

is

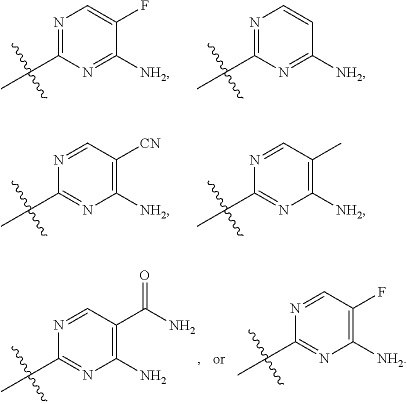

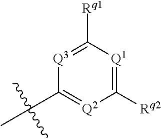

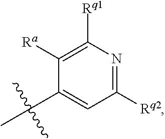

In some embodiments where is

wherein R^{a} is defined as above, and

R^{q1} is H, NH₂, (C₁-C₆)alkyl, (C₁-C₆)alkylene-OH, (C₁-C₆)alkylene-O(C₁-C₆)alkyl, (C₁-C₆)alkylene-NH₂, (C₁-C₆)alkylene-NH(C₁-C₆)alkyl, (C₁-C₆)alkylelene-N(C₁-C₆)alkyl)₂, (C₁-C₆)alkylene-NHSO₂-(C₁-C₆)alkyl, (C₁-C₆)alkylene-NH(C=O)-(C₁-C₆)alkyl, NH₂, NH(C₁-C₆)alkyl, N((C₁-C₆)alkyl)₂, (C₁-C₆)alkylene-NHSO₂-(C₁-C₆)alkyl, (C₁-C₆)alkylene- NH(C=O)-(C₁-C₆)alkyl, -(C=O)-NH₂, —(C=O)-NH(C₁-C₆)alkyl, -(C=O)—NH (C₁-C₆)alkyl))₂, -NHSO₂-(C₁C₆)alkyl, -CN, or (C₁-C₆)alkylene-(C₃-C₇)heterocyclo; and

R^{q2} is H, (C₁-C₆)alkyl, (₁-C₆)alkenyl, halo, halo(C₁-C₆)alkyl, NH₂, NH(C₁-C₆)alkyl, N((C₁-C₆)alkyl)₂.

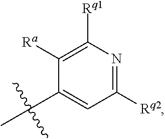

In some embodiments

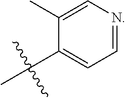 [SMILES: *c1ccncc1C]

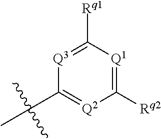

In some embodiments where

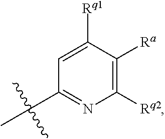

wherein R^{a} is defined as above; and

R^{q1} is H, NH₂, (C₁-C₆)alkyl, (C₁-C₆)alkylene-OH, (C₁-C₆)alkylene-O(C₁-C₆)alkyl, (C₁-C₆)alkylene-NH₂, (C₁-C₆)alkylene-NH(C₁-C₆)alkyl, (C₁-C₆)alkylelene-N(C₁-C₆)alkyl)₂, (C₁-C₆)alkylene-NHSO₂-(C₁-C₆)alkyl,(C₁-C₆)alkylene-NH(C=O)-(C₁-C₆)alkyl, NH₂, NH(C₁- C₆)alkyl, N((C₁-C₆)alkyl)₂, (C₁-C₆)alkylene-NHSO₂-(C₁-C₆)alkyl, (C₁-C₆)alkylene- NH(C=O)-(C₁-C₆)alkyl, -(C=O)-NH₂, -(C=O)-NH(C₁-C₆)alkyl, -(C=O)-NH (C₁-C₆)alkyl))₂, NHSO₂-(C₁-C₆)alkyl, -CN, or (C₁-C₆)alkylene-(C₃-C₇)heterocyclo; and

R^{q2} is H, (C₁-C₆)alkyl, (C₁-C₆)alkenyl, halo, halo(C₁-C₆)alkyl, NH₂, NH(C₁-C₆)alkyl, N((C₁-C₆)alkyl)2.

In some embodiments,

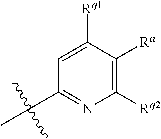

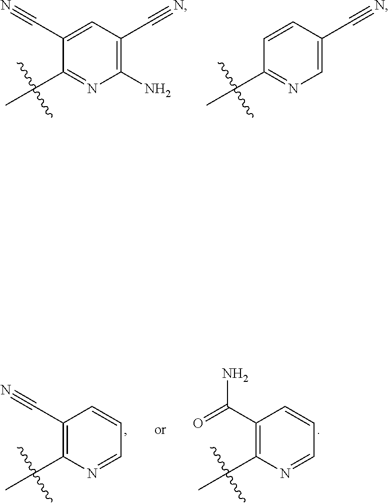 [SMILES: *c1nc(N)c(C#N)cc1C#N ; *c1ccc(C#N)cn1 ; *c1ncccc1C#N ; *c1ncccc1C(N)=O]

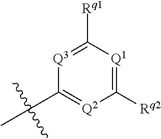

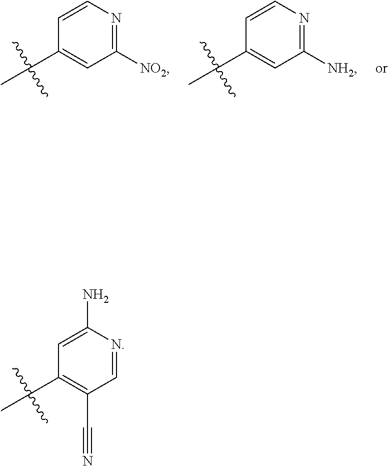 [SMILES: *c1ccnc([N+](=O)[O-])c1 ; *c1ccnc(N)c1 ; *c1cc(N)ncc1C#N]

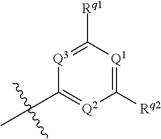

In some embodiments,

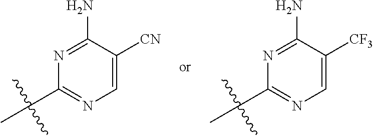 [SMILES: *c1ncc(C#N)c(N)n1 ; *c1ncc(C(F)(F)F)c(N)n1]

In some embodiments,

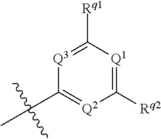

In some embodiments,

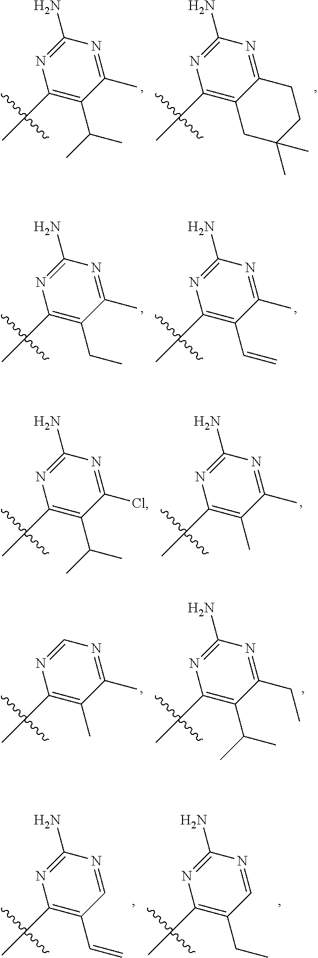

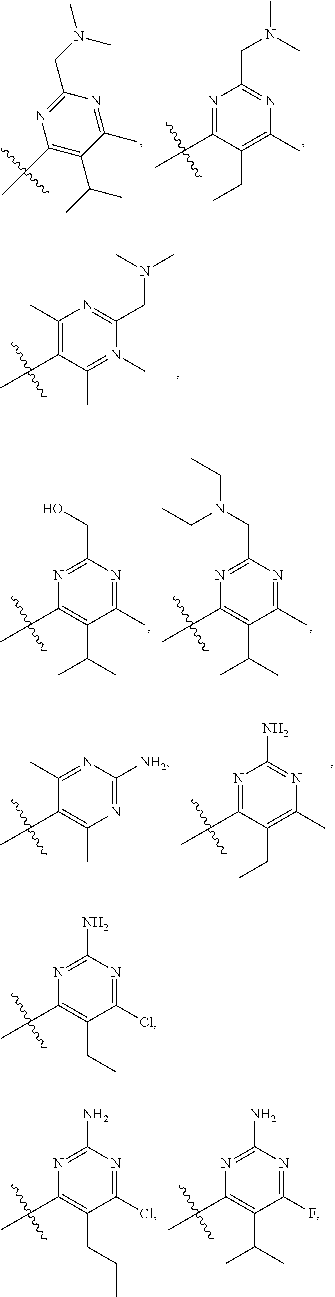

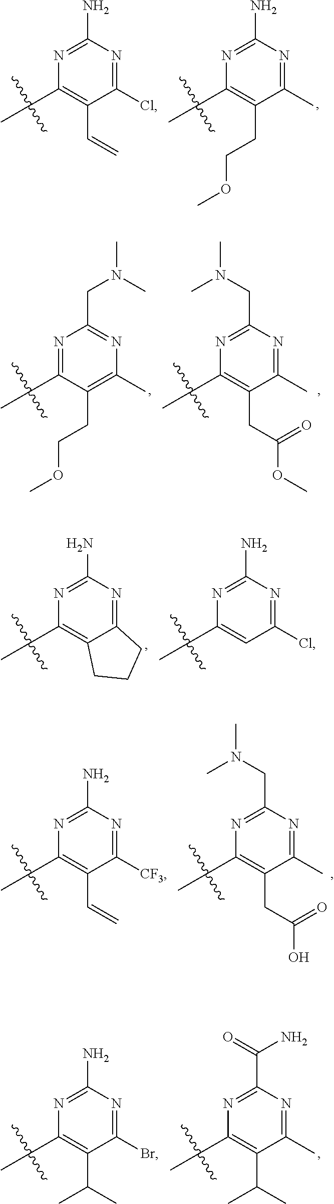

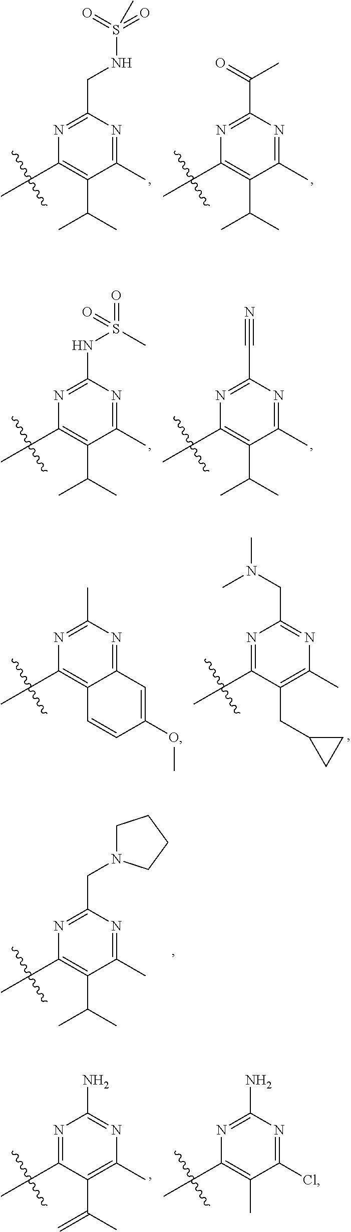

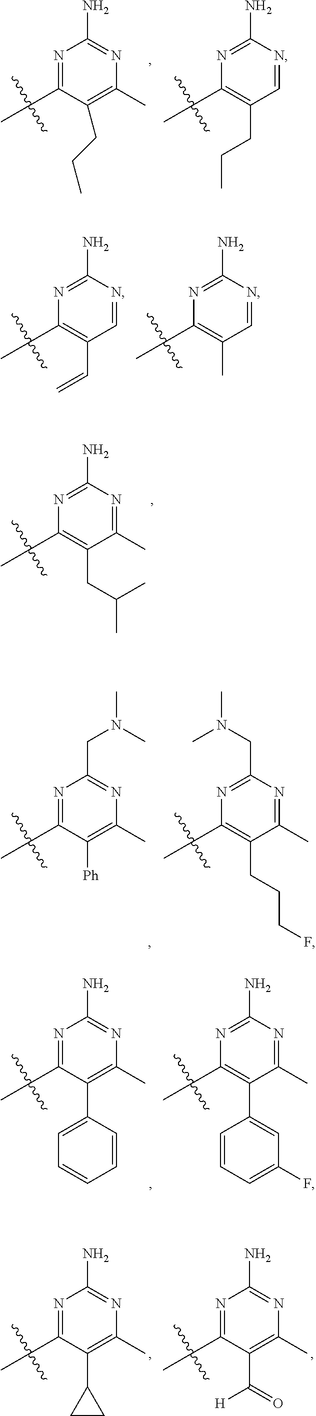

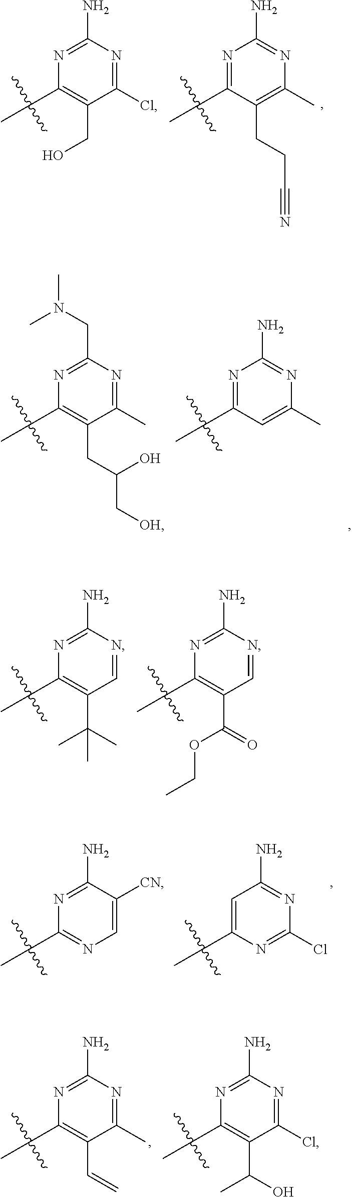

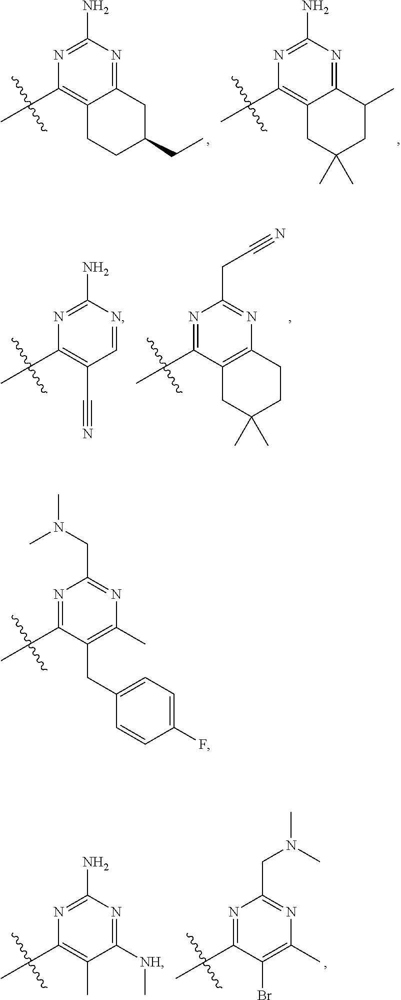

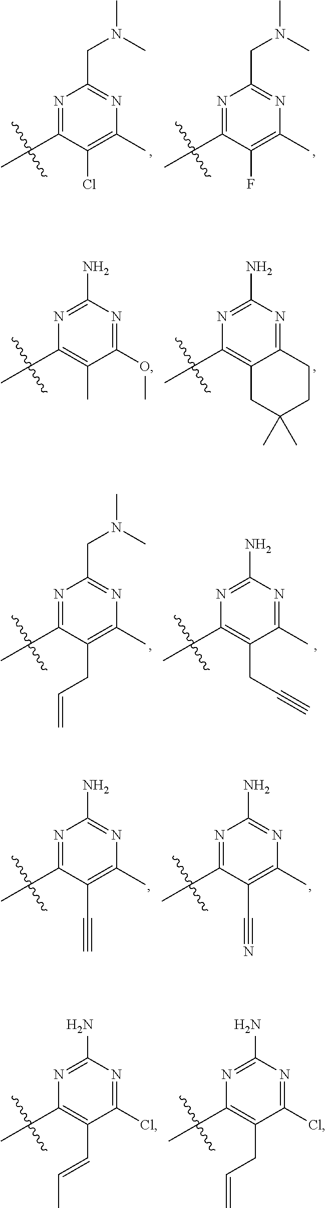

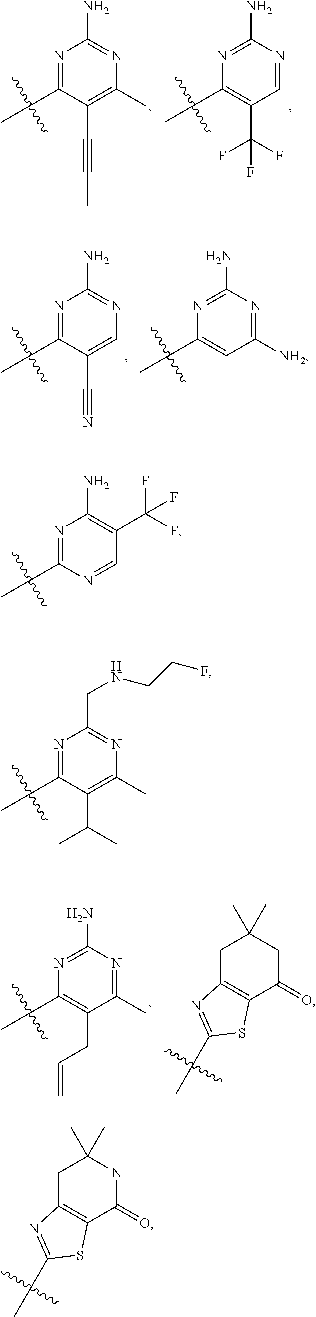

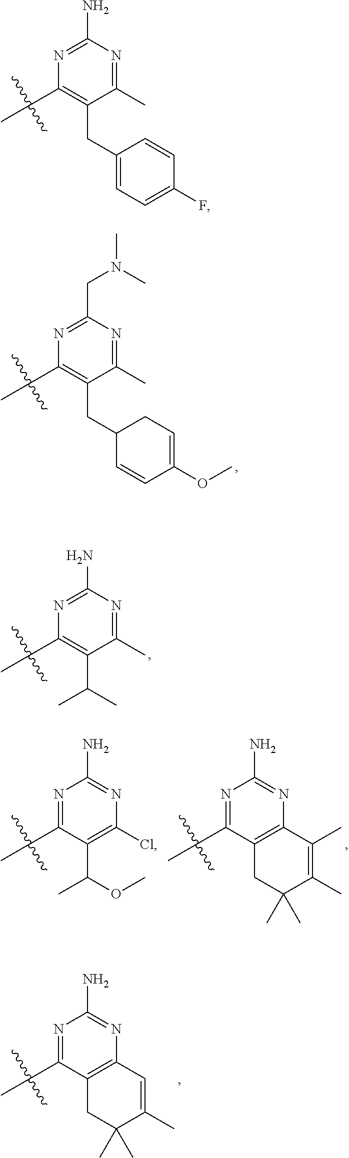

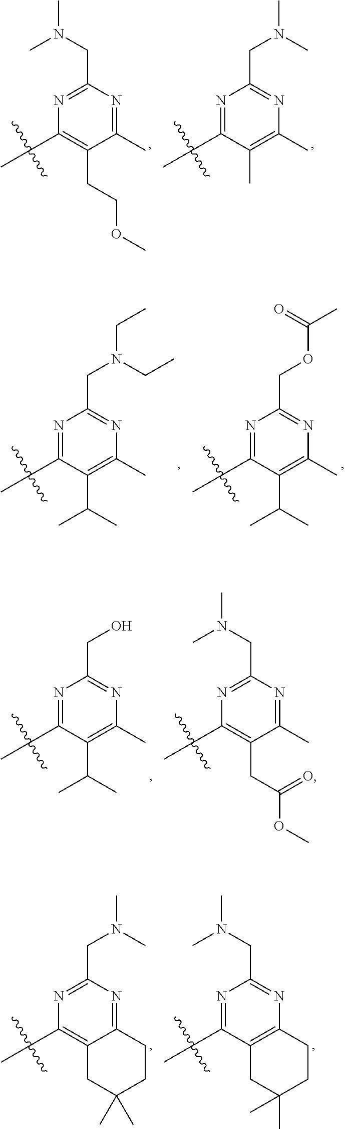

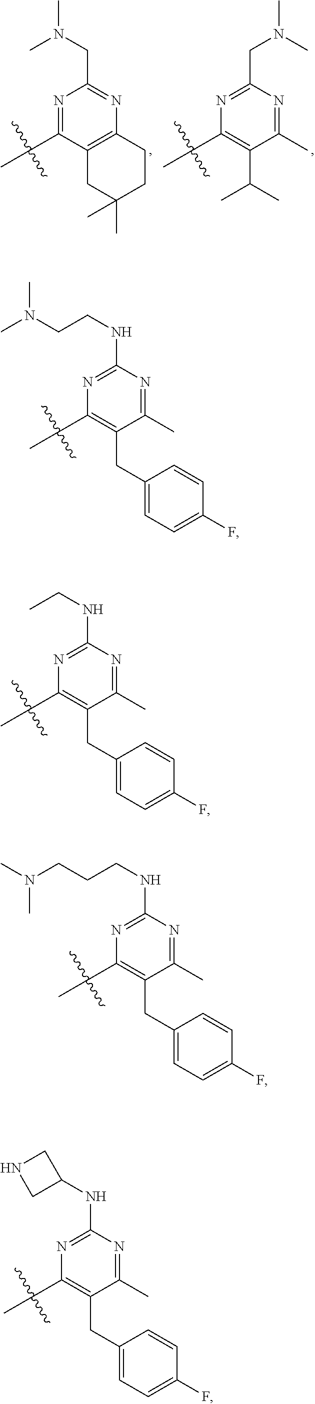 [SMILES: *c1nc(CN(C)C)nc2c1CC(C)(C)CC2 ; *c1nc(CN(C)C)nc(C)c1C(C)C ; *c1nc(NCCN(C)C)nc(C)c1Cc1ccc(F)cc1 ; *c1nc(NCC)nc(C)c1Cc1ccc(F)cc1 ; *c1nc(NCCCN(C)C)nc(C)c1Cc1ccc(F)cc1 ; *c1nc(NC2CNC2)nc(C)c1Cc1ccc(F)cc1]

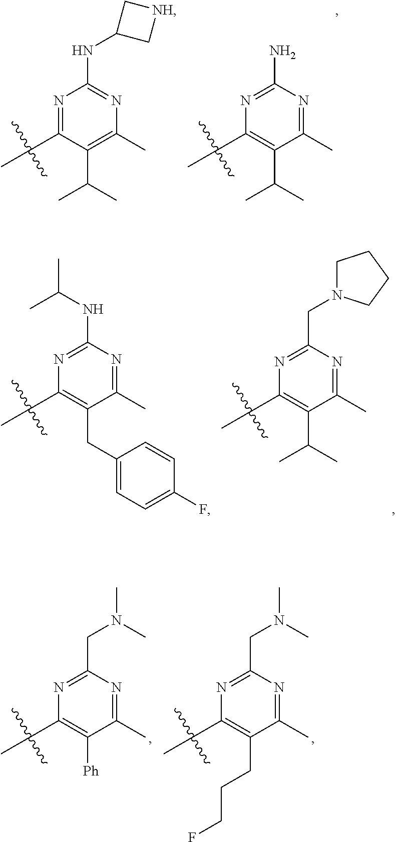

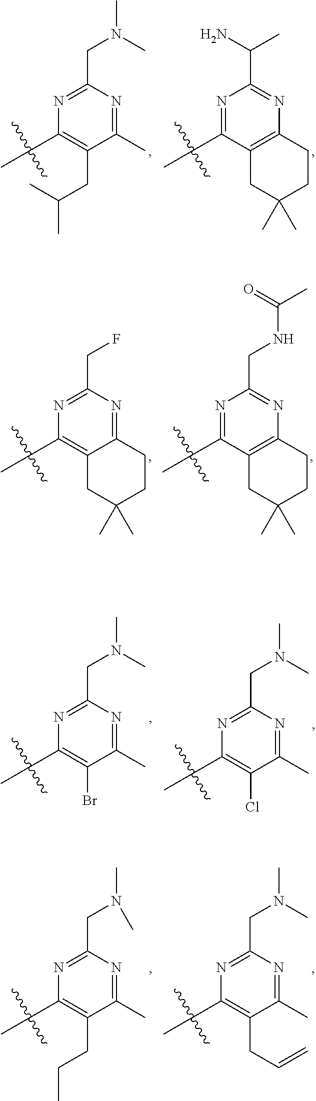

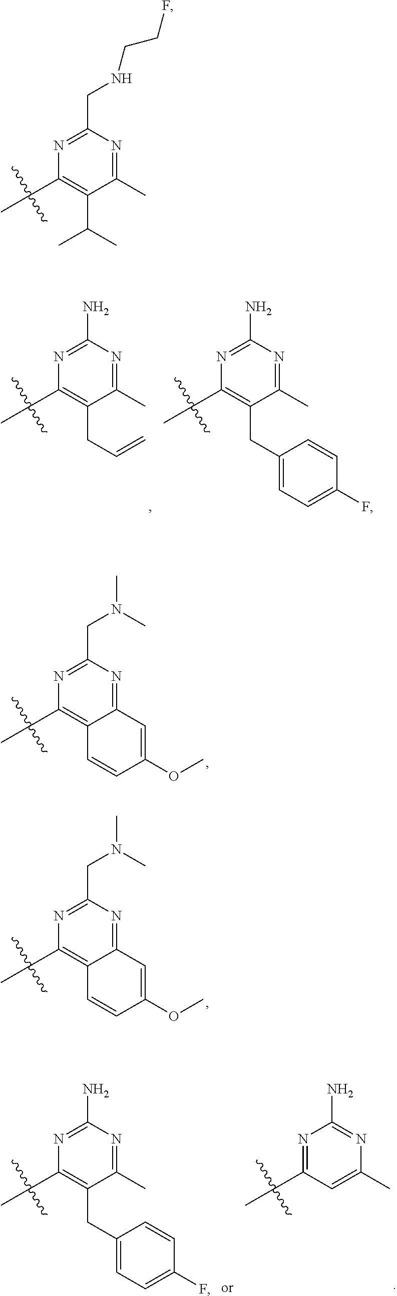

In another embodiment, the compound of Formula I is a compound of formula II(a) or II (b), wherein the variables can have any of the definitions provided herein.

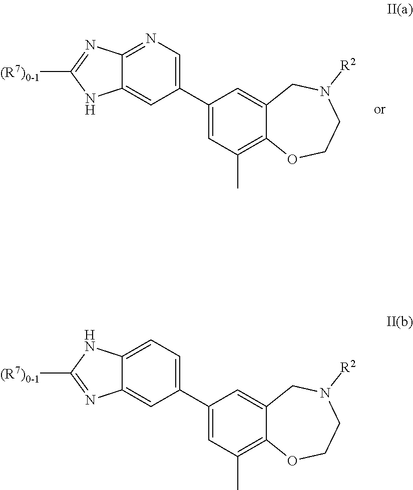

In some embodiments of Formula II(a) or II(b). R⁷ is hydrogen, (C₁₋₃)alkyl, cyclopropyl, fluoromethyl, difluoromethyl, trifluoromethyl, or NH². In some embodiments of Formula II(a) or II(b), R⁷ is methyl or NH₂. In these and other embodiments, R² can be any

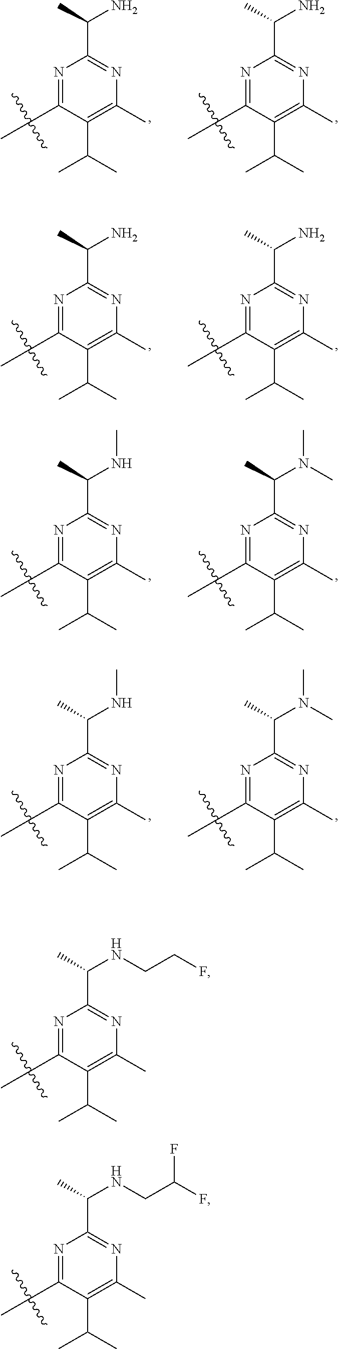

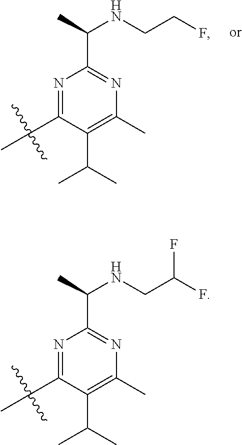 [SMILES: *c1nc([C@@H](C)NCCF)nc(C)c1C(C)C ; "or" ; *c1nc([C@@H](C)NCC(F)F)nc(C)c1C(C)C]

of the definitions provided herein. In some embodiments, R² is

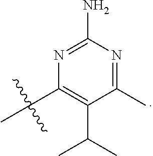 [SMILES: *c1nc(N)nc(C)c1C(C)C]

More particularly, R² is

In another embodiment, the compound of Formula I is a compound of formula II(a) or II(b), wherein the variables can have any of the definitions provided herein.

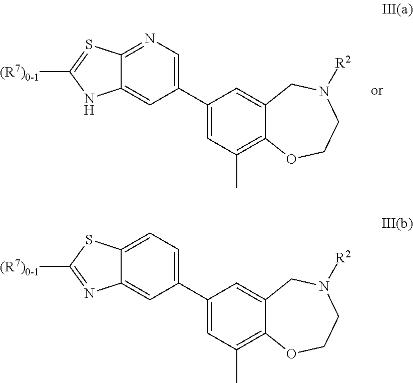

In some embodiments, of Formula III(a) or III(b). R⁷ is hydrogen. (C₁C₃)alkyl, cyclopropyl, fluoromethyl, difluoromethyl, trifluoromethyl, or NH₂. In some embodiments of Formula II(a) or II(b). R⁷ is methyl or N₂. In these and other embodiments, R² can be any

of the definitions provided herein. More particularly, R² is

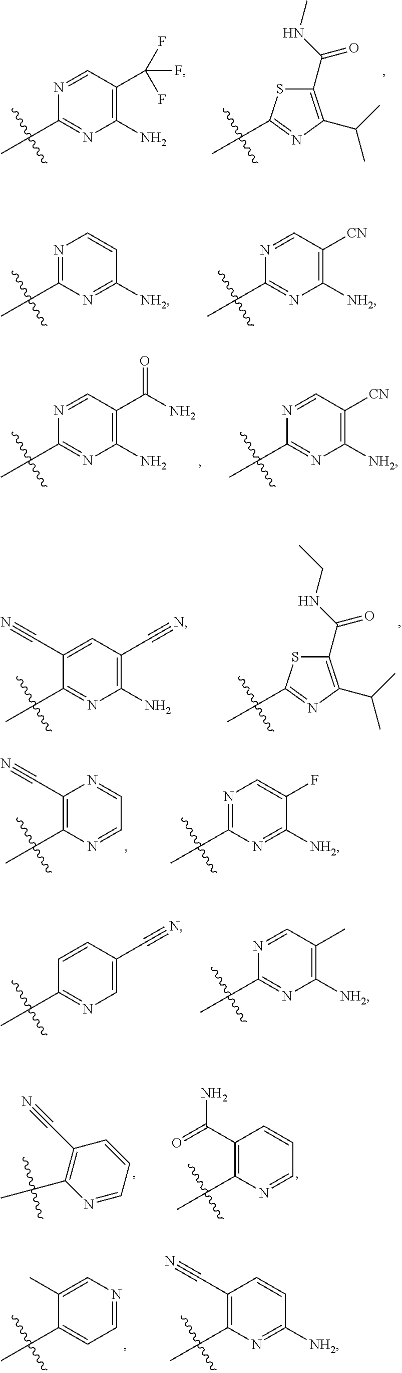

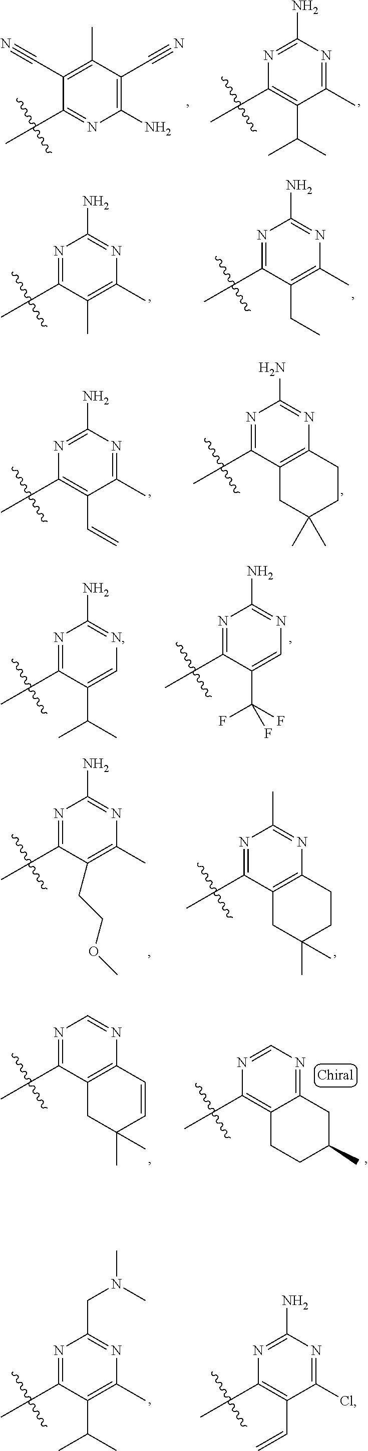

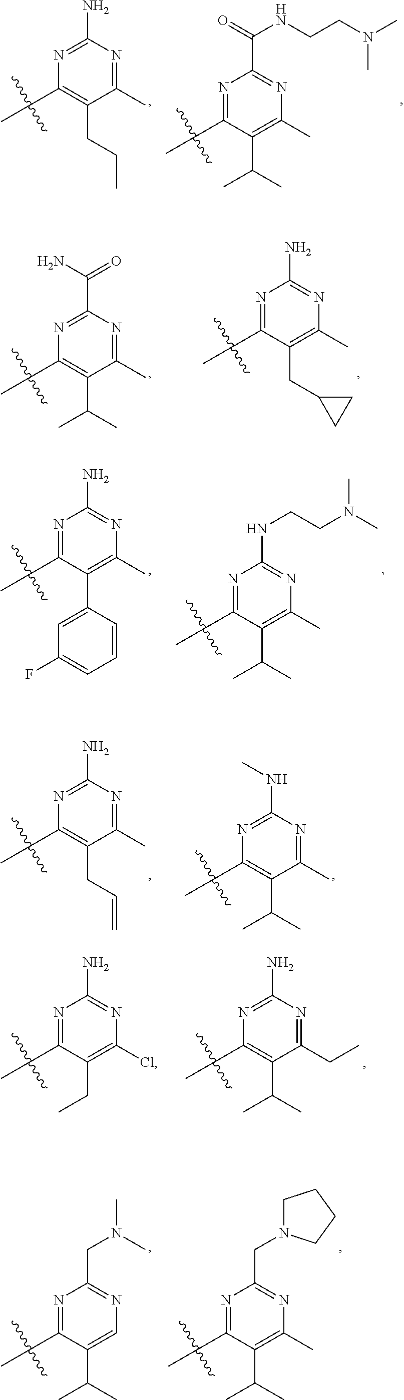

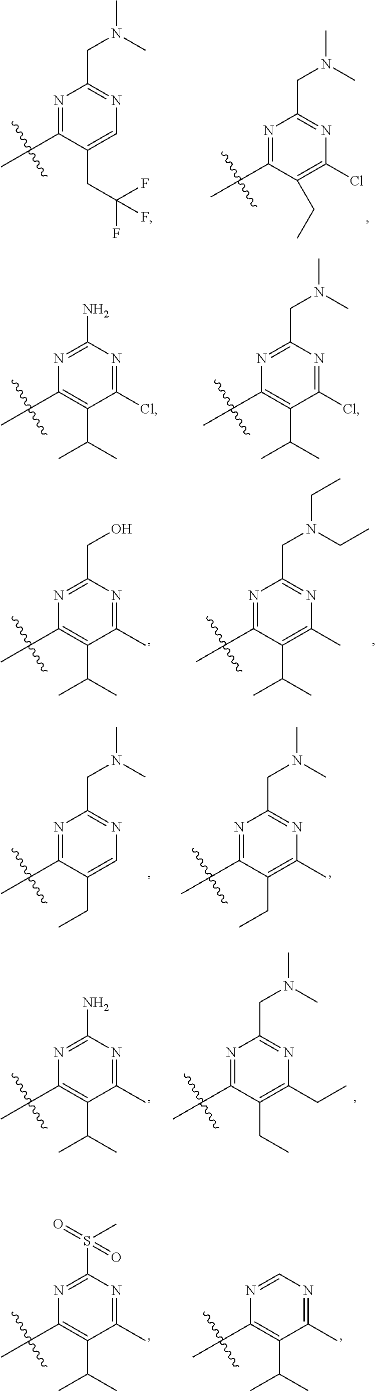

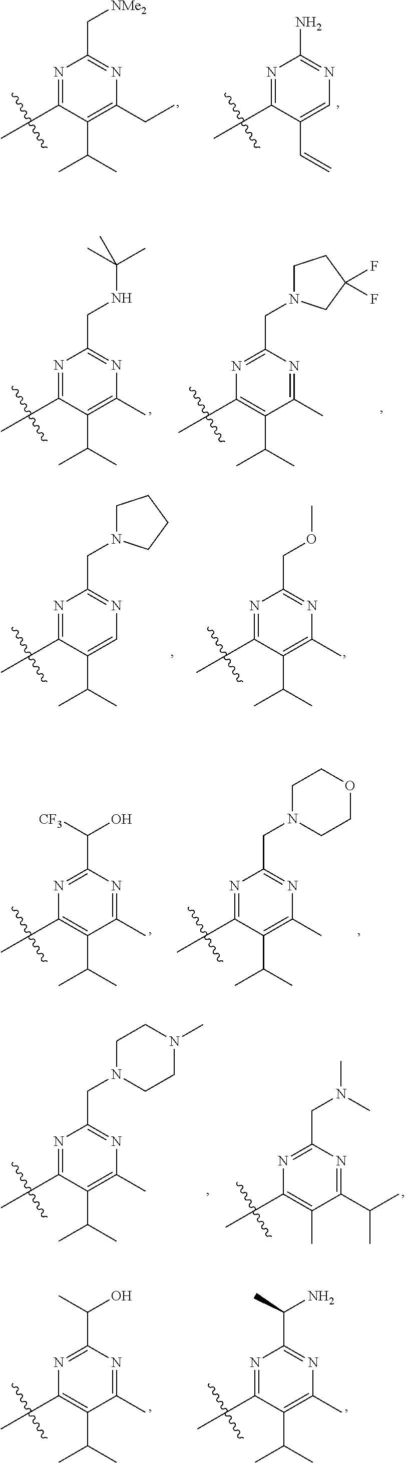

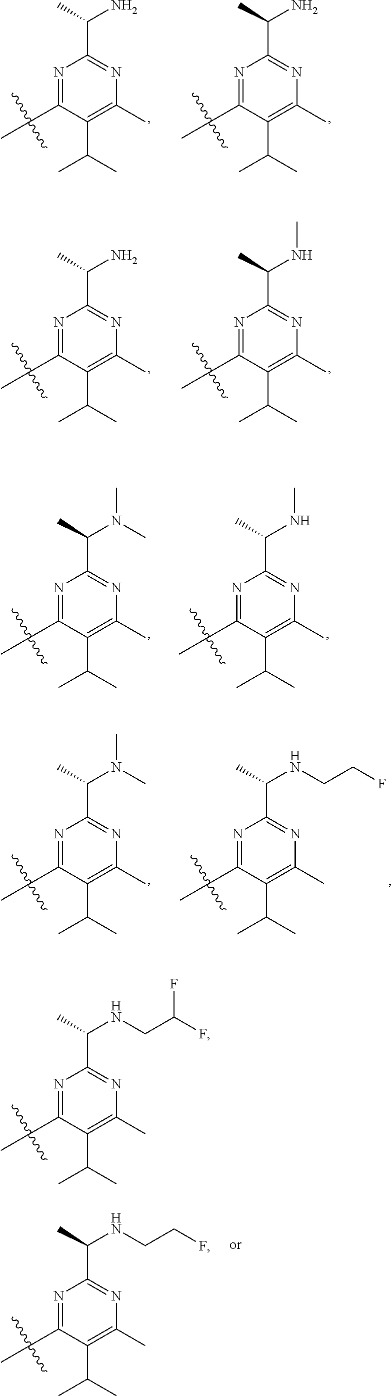

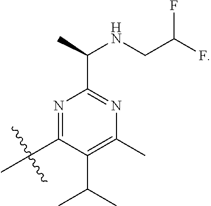 [SMILES: *c1nc([C@@H](C)NCC(F)F)nc(C)c1C(C)C]

In another embodiment, the compound of Formula I is a compound of formula IV(a) or IV(b), wherein the variables can have any of the definitions provided herein.

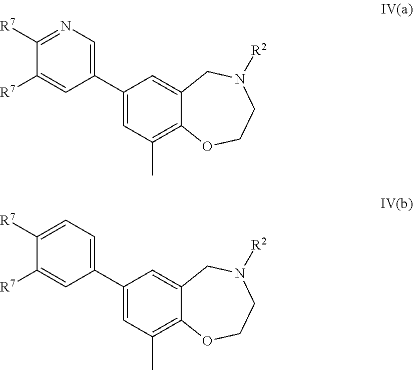

In some embodiments of Formula IV(a) and IV(b), one or both of the R⁷ groups are optionally present. In particular, when both R⁷ groups are present, one R⁷ is NH₂ chloro, hydroxy, —CO₂Me, or methoxy and the other R⁷ is +SO₂NH₂, —NHSO₂Me, or methoxy and

R² is

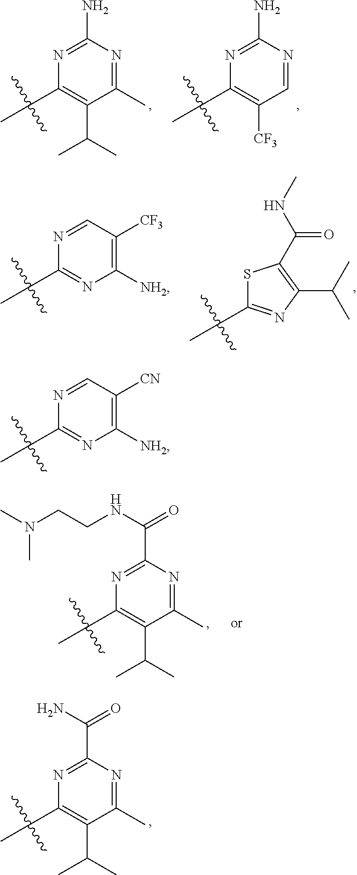

In other embodiments, the compound of Formula IV(a) or IV(b) is a compound of Formula IV(a1) or IV(b1), wherein the variables can have any of the definitions provided herein.

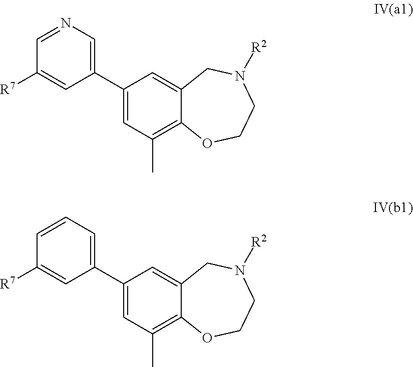

In some embodiments of Formula IV(a1) and IV(b1), R⁷ is —OH, —N₂, SO₂NH₂.

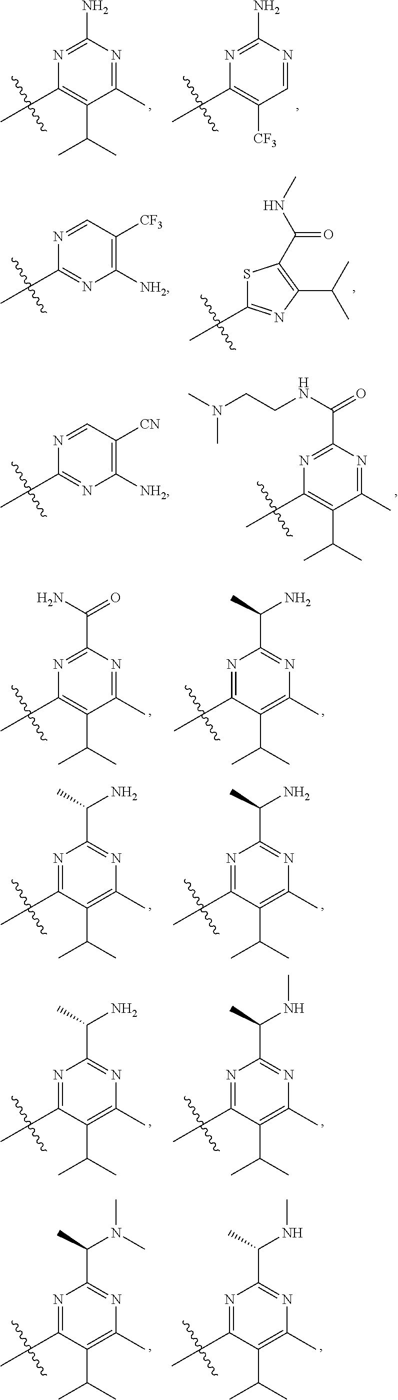

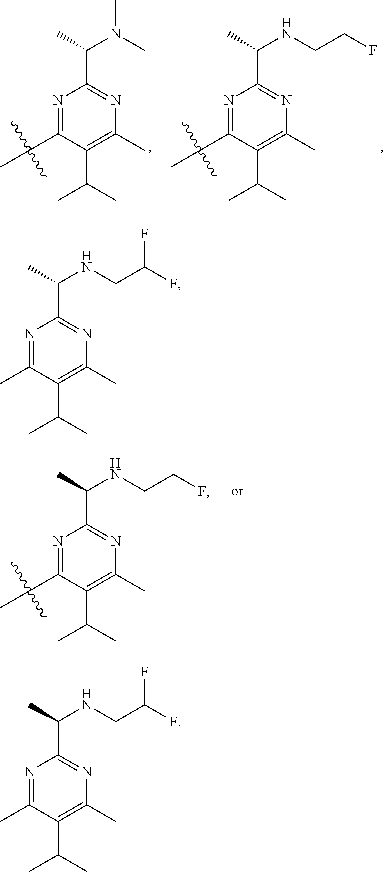

—NHSO₂ME, or methoxy, and R² is

In other embodiments, the compound of Formula IV(a) or IV(b) is a compound of Formula IV(a2) or IV(b2), wherein the variables can have any of the definitions provided herein.

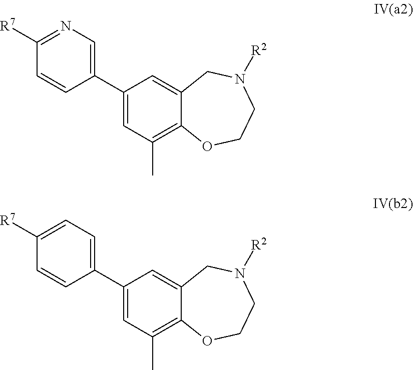

In some embodiments of Formula IV(a2) and IV(b2). R⁷ is NH₂, chloro, hydroxy,

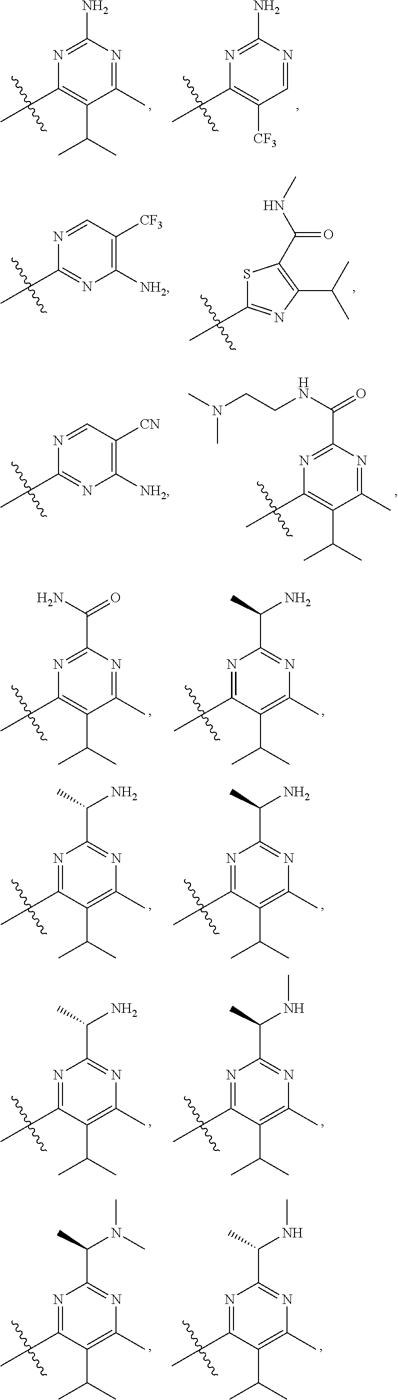

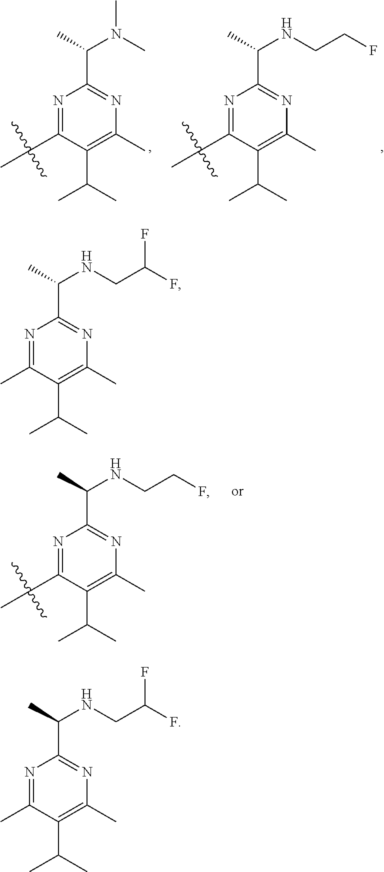

—CO₂Me, or methoxy, and R² is

In other embodiments, the compound of Formula IV(a) or IV(b) is a compound of Formula IV(b3), wherein R⁷ is joined together with the carbons to which they are attached lo form a 5 or 6-membered heterocycloalkyl group and R² can have any of the definitions provided herein.

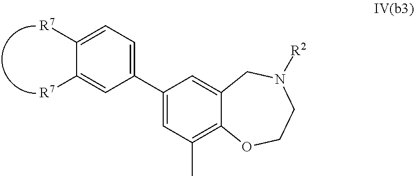

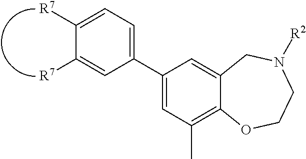

In some embodiments of Formula IV(b3) is

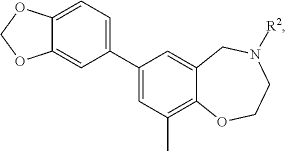

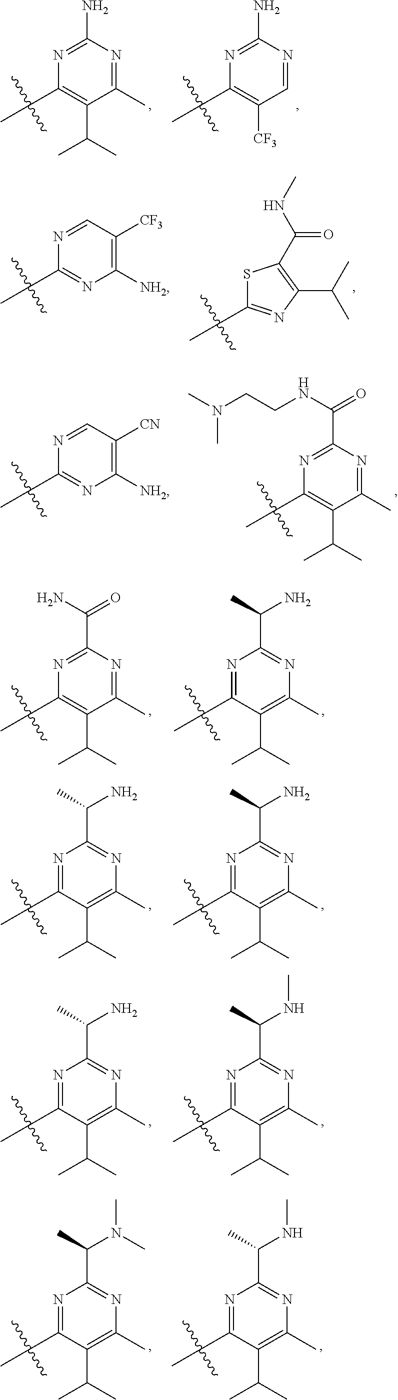

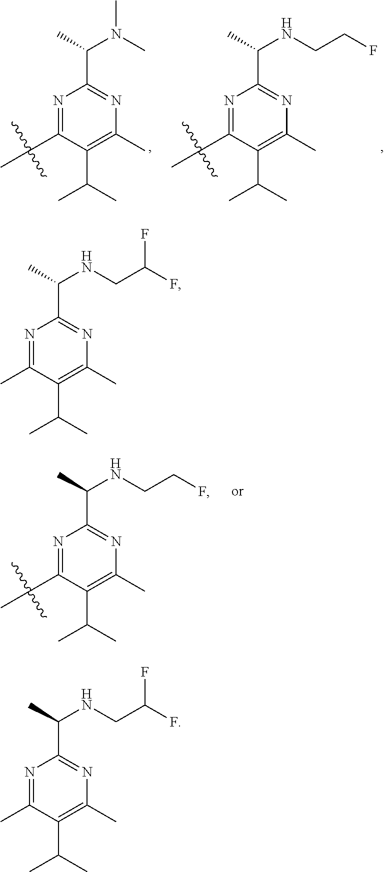

### and R² is

In some embodiments of Formula IV(a2) and IV(b2). R⁷ is fluoro, chloro.

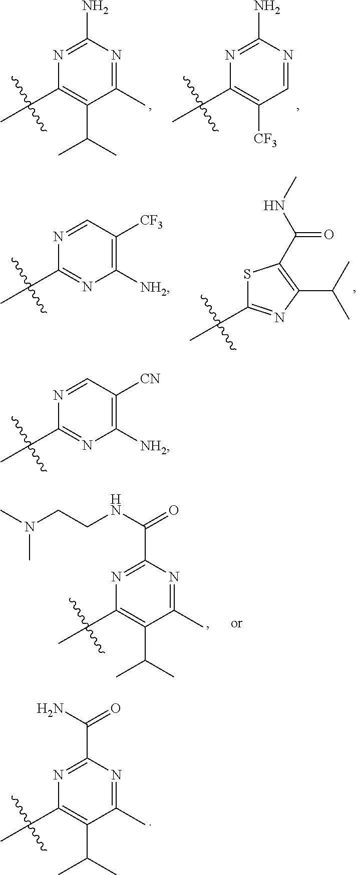

methoxy, NH₂, chloro, hydroxy, —CO₂Me, or methoxy, and R² is

In some embodiments of Formula IV(a2) and IV(b2). R⁷ is fluoro, chloro,

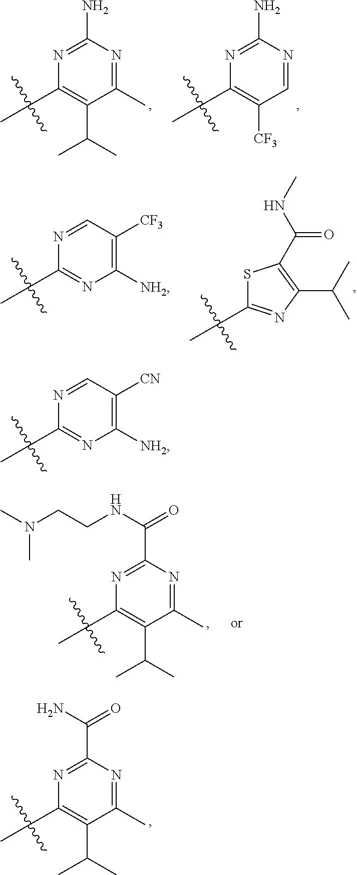

mthoxy, NH₂, chloro, hydroxy, —CO₂Me, or methoxy, and R² is

In another embodiment, the compound of Formula I is a compound of formula V(a), V(b), V(e), or V(d), wherein the variables can have any of the definitions provided herein.

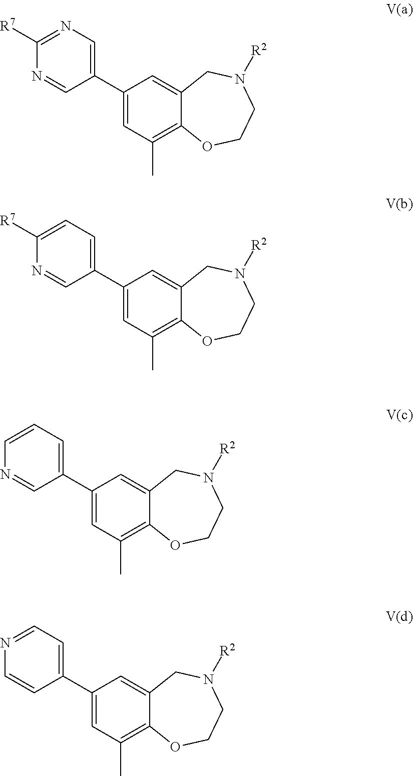

In another embodiment, the compound of Formula I is a compound of formula VI(a) orVI(b), wherein the variables can have any of the definitions provided herein.

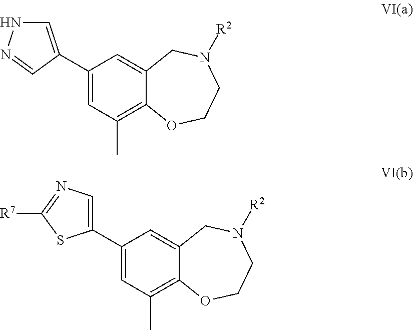

In another embodiment, the compound of Formula I is a compound of Formula VI(a) or VI(b), wherein the variables can have any of the definitions provided herein.

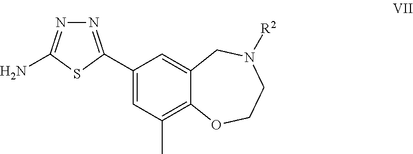

In another embodiment, the compound of formula I, I(a), I(b), I(a), I(b), IV(a), IV(b), V(a), V(b), V(c), V(d), VI(a), or VI(a) is a compound of formula VIII:

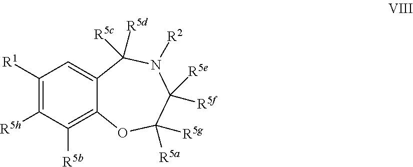

or a single stereoisomer or mixture of stereoisomers thereof and additionally optionally as a pharmaceutically acceptable salt thereof, where
R¹ is aryl optionally substituted with one, two, or three R⁶ groups; or heteroaryl optionally substituted with one, two, or three R⁷;
R² is heteroaryl substituted wilh R³, R³a, R^{3b}, R^{3c}, and R^{3d};
R³, R^{3a}, R^{3b}, R^{3c}, and R^{3d} are independently hydrogen, cyano, alkyl, alkenyl, halo, haloalkyl, hydroxyalkly, alkoxyalkyl, cyanoalkyl, -SR¹², -S(O)₂R²⁰, -C(O)OR⁴, -C(O)NHR⁴halocarbonyl, -NR¹¹R^{11a}, -OR^{11a}, optionally substituted phenyl, optionally substituted phenylalkyl, optionally substituted cycloalkyl, optionally substituted cycloalkylalkyl, optionally substituted heterocycloalkyl, optionally substituled heterocycloalkylalkyl., optionally substituted heteroaryl, optionally substituted heteroarylalkyl, or alkyl substituted with one or two R¹⁶; or
two of R³, R^{3a}, R^{3b}, R^{3c}, and R^{3d}, when attached to the same carbon, form an optionally substituted cycloalkyl, optionally substituted aryl, or an optionally substituted heteroeycoalkyl; or optionally substituled heteroaryl, and the other of R³, R^{3a}, R^{3b}, R^{3c}, and R^{3d} are independently hydrogen, cyano, alkyl, alkenyl, halo, haloalkyl, hydroxyalkyl, alkoxyalkyl, cyanoalkyl, -SR¹², -S(O)₂R^{2d}, -C(O)OR⁴,halocarbonyl, -NR¹¹R^{11a}, OR^{11a}, optionally substituted phenyl, optionally substituted phenylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶;
R⁴ is alkyl, alkenyl, alkynyl, hydroxyalkyl, alkoxyalkyl, haloalkyl, aminoalkyl, alkylaminoalkyl, dialkylaminoalkyl, benzyl, or optionally substituted heterocycloalkylalkyl;
R^{5a} and R^{5c} are independently hydrogen or alkyl;
R^{5h} is hydrogen or halo;
R^{5h} (₁₋₃)alkyl or halo(C₁₋₃)alkyl;
R^{5d}, R^{5c}, R^{5f}, and R^{5g} are hydrogen;
each R⁶, when R⁶ is present, is independently nitro; cyano; halo; alkyl; alkenyl; alkynyl; haloalkyl; -OR^{8a}; -NR⁸R^{8a}; —C(O)NR⁸R^{8a}; —S(O)₂R⁸; —NR⁸C(O)OR⁹; —NR⁸C(O)R⁹; —NR⁸S(O)₂RS^{8a};—NR⁸C(O)NR^{8a}R⁹; carboxy, —C(O)OR⁹; halocarbonyl; alkylcarbonyl; alkyl substituted with one or two —C(O)NR⁸RS^{8a}; heteroaryl optionally substituted with 1, 2, or 3 R¹⁴; or optionally substituted heterocycloalkyl; or
two R⁶, together with the carbons to which they are attached, form an optionally substituted 3, 4, 5, or 6-membered cycloalkyl or heterocycloalkyl;

each R⁷, when R⁷ is present, is independently oxo; nitro; cyano; alkyl; alkenyl; alkynyl; halo; haloalkyl; hydroxyalkyl; alkoxyalkyl; —OR^{8a}; —SR¹³; —S(O)R², —S(O)₂R^{13a}; —NR⁸R^{8a}; —C(O)NR⁸R^{8a}; —NR⁸C(O)OR⁹; —NR⁸C(O)R⁹; —NR⁸S(O)₂R^{8a}; —NR⁸C(O)NY^{8a}R⁹; —C(O)OR⁹; halocarbonyl; alkylcarbonyl; —S(O)₂NR⁸R⁹; alkylsulfonylalkyl; alkyl substituted with one or two —NR⁸R^{8a}; alkyl substituted with one or two —NR^{8C}(O)R^{8a}; alkyl substituted with one or two —NR⁸C(O)OR⁹; alkyl substituted wiih one or two —S(O)₂R^{13a}; optionally substituted cycloalkyl; optionally substituted cycloalkylalkyl; optionally substituted heterocycloalkyl; optionally substituted heterocycloalkylalkyl; optionally substituted phenyl; optionally substituted phenylalkyl; optionally substituted heteroaryl; or optionally substituted hetcroarylalkyl;

each R⁸, R¹¹, R¹⁵, R¹⁷, and R¹⁸ are independently hydrogen, NH₂; NH(alkyl), N(alkyl)₂, alkyl, alkenyl, alkynyl, hydroxyalkyl, alkoxyalkyl, or haloalkyl;
each R^{8a}, R^{11a}, and R^{15a} are independently hydrogen, alkyl, alkenyl, alkynyl, haloalkyl, hydroxyalkyl, cyanoalkyl, aminoalkyl, alkylaminoalkyl, dialkylaminoalkyl, alkoxyalkyl, carboxyalkyl. optionally substituted cycloalkyl. optionally substituted cycloalkylalkyl, optionally substituted heterocycloalkyl, optionally substituted heterocycloalkylalkyl, optionally substituted phenyl, optionally substituted phenylalkyl, optionally substituted heteroaryl, or optionally substituted heteroarylalkyl;
R⁹ is hydrogen; alkyl; alkenyl; alkynyl; hydroxyalkyl; alkoxyalkyl; aminoalkyl; alkylaminoalkyl; dialkylaminoalkyl; haloalkyl; hydroxyalkyl substituted with one, two, or three groups which are independently halo, amino, alkylamino, or dialkylamino, alkyl substituted with one or two aminocarbonyl; optionally substituted phenyl; optionally substituted phenylalkyl, optionally substituted cycloalkyl; optionally substituted cycloalkylalky; optionally substituted heteroaryl; optionally substituted heterarylalkyl; optionally substituted heterocycloalkyl; or optionally substituted hetcrocycloalkylalkyl;
R¹² is alkyl or optionally substituted phenylalkyl;
R¹³ is alkyl, hydroxyalkyl, or haloalkyl; and
R^{13a} is hydroxy, alkyl, haloalkyl, hydroxyalkyl, or heterocycloalkyl optionally substituted with one or two groups which are independently halo, amino, alkylamino,dialkylamino, hydroxy, alkyl, or hydroxyalkyl;:
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbpnyl, alkylaminocarbonyl, dialkylaminocarbonyl, or optionally substituted phenyl;

each R¹⁶ is indepependently halo, —NR¹¹R^{11a}, —NR¹⁵S(O)R^{15a}, -13 OC(O)R¹⁷, or —OR¹⁸; and

R²⁰ alkyl, haloalkyl, hydroxyalkyl, amino, alkylamino, dialkylamino, or heterocycloalkyl.

Another embodiment provides a pharmaceutical composition which comprises 1) a compound, as single stereoisomer or mixture of stereoisomers thereof, according to any one of Formula I, (I(a); I(b),. I(b2), I(c1), I(c2), I(d1), I(:d2), I(e), I(e1), I(f), I(g), I(h), I(j), I(k), I(m), I(n), I(p), I(q), I(r), I(s), and I(t) or according to any one of the above embodiments, optionally as a pharmaceutically acceptable salt thereof, and 2) a pharmaccutically acceptable carrier, excipient, and/or diluent thereof.

Another embodiment is a method of treating disease, disorder, or syndrome where the disease is associated with uncontrolled, abnormal, and/or unwarned cellular activities effected directly or indirectly by PI3K and/or mTOR, which method comprises administering to a human in need thereof a therapeutically effective amount of a Compound of any of Formula I,. (I(a), I(b1) I(b2), I(c1)., I(c2), I(d1), I(d2), I(e), I(e1), I(f), I(g), I(h), I(j), I(k), lI(m), I(n), I(p), I(p), I(q), I(s), and I(t), a Compound of any one of the above embodiments, or a Compound from Table I, optionally as a pharmaceutically acceptable salt or pharmaceutical composition thereof. In another embodiment the disease is cancer. In another embodiment, the disease is cancer and the Compound is of Formula I(a) or a Compound from Table 1.

### Embodiment (G)

Another embodiment is directed to a method of treating a disease, disorder, or syndrome which method comprises administering to a patient a therapeutically effective amount of a Compound of any of Formula I, (I(a), I(b1), I(b2), I(c 1), I(c2), I(d1), I(d2), I(e), I(e1), I(f),. I(g), I(h), I(j), I(k), I(m), I(n), I(p), I(q), I(r), I(s), and I(t), a Compound of any one of the above embodiments, or a Compound from Table 1, optionally as a pharmacculically acceptable salt thereof, or a pharmaceutical composition comprising as therapeutically effective amount of a Compound of Formula I, (I(a), I(b1), I(b2), I(c1), I(c2), I(d1), I(d2), I(e), I(e1), I(f), I(g), I(h), I(j), I(k), I(m), I(n), I(p), I(q), I(r), I(s), and I(t), a Compound of any one of the above embodiments, or a Compound from Table 1, and a pharmacculically acceptable carrier, excipient, or diluent. In another embodiment the disease is cancer.

In another embodiment of any of the embodiments of Embodiment (G), the cancer is breast cancer, mantle cell lymphoma, renal cell carcinoma, acute myelogenous leukemia, chronic myelogenous leukemia, NPM/ALK-transfonned anaplastic large cell lymphoma, diffuse large B cell lymphoma, rhabdomyosarcoma, ovarian cancer, endometrial cancer, cervical cancer, non small ceil lung carcinoma, small cell lung carcinoma, adenocarcinoma, colon cancer, rectal cancer, gastruic carcinoma, hepatocellular carcinoma, melanoma, pancreatic cancer, prostate carcinoma, thyroid carcinoma, anaplastic large cell lymphoma, hemangioma, glioblastoma, or head and neck cancer.

All Compounds in Table 1 were lested in the assays described in Biological Examples 1and 3.

### Embodiments (V)

In one embodiment the Compound of the Invention has an PI3K-aIpha-inhibiiory activity of about 2.0 μM or less and is inactive for mTOR (when tested at a concentration of 3.0 μM or greater) or is selective for PI3K-alpha over mTOR by about 5—fold or greater, about 7—fold or greater, or about 10—fold or greater. In anolher embodiment the Compound of the Invention has an PI3K—alpha-inhibitory activity of about 1.0 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the Compound of the Invention has an PI3K-alpha-inhibitory activity of about 0.5 μM or less and is inactive for mTOR (when tested at a concentration of 2.0 μM or greater) or is selective for PI3K-alpha over mTOR by about 5-fold or greater, about 7-fold or greater, or about 10-fold or greater. In another embodiment the Compound of the Invention has an PI3K-alpha-inhibilory activity of about 0.3 μM or less and is inactive for mTOR (when tested at a concentration of 2.0 μM or greater) or is selective for PI3K-alpha over in mTOR by about 5-fold or greater, about 7-fold or greater, or about 10- fold or greater. In another embodiment the Compound of the Invention has all PI3K-alpha-inhibitory activity of about 0.2μM or less and is selective for PI3K-alpha- over MTOR by about 5-fold of greater, about 7-fold or greater or about 10-fold or greater. In another embodiment the Compound of the Invention has an PI3K-alpha-inhibitory activity of about 0.1 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.05 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.025 μM or less and is selective for PI3K-alpha over mTOR by about 5-fold or greater, about 7-fold or greater, or about 10-fold or greater. In another embodiment the Compound of the Invention has an PI3K-alpha-inhibitory activity of about 0.01 μM or less and is selective for PI3K-alpha over mTOR by about 5-fold or greater, about 7-fold or greater, or about 10-fold or greater.

### Embodiments (W)

In one embodiment the Compound of the Invention has an PI3K-alpha-inhibiiory activity of about 2.0 μM or less and an mTOR-inhibitory activity of about 2.0 μM or less and the selectivity for one of the targets over the other does not exceed 3-fold. In another embodiment the Compound of-the Invention has an PI3K-alpha-inhibitory activity of about 1.0 μM or less and an mTOR-inhibitory activity of about 1.0 μM or less and the selectivity for one of the targets over the other does not exceed 3-fold. In another embodiment the Compound of the Invention has an PI3K-alpha-inhibitory activity of about 0.5 μM or less and an mTOR-inhibitory activity of about 0.5 μM or less and the selectivity for one of the targets over the other does not exceed 3-fold. In another embodiment the Compound of the Inventionii has an PI3K-alpha-inhibitory activity of about 0.3 μM or less and an mTOR-inhibitory activity of about 0.3 μM or less and the selectivity for one of the targets over the other does not exceed 3-fold. In another embodiment the Compound of the Invention has an PI3 K-alpha-inhibiiory activity of about 0.15 μM or less and an mTOR-inhibitory activity of about 0.15 μM or less and the selectivity for one of the targets over the other does not exceed 2-fold. In another embodiment the Compound of ihe Invention has an PI3K-alpha-inhibitory activity of about 0.1 μM or less and an mTOR-inhibitlory activity of about 0.1 μM or less. In another embodiment the Compound of the Invention has an PI3K-alpha-inhibitory activity of about 0.05 μM or less and an mTOR-inhibitory activity of about 0.05 μM or less. In another embodiment the Compound of the Invention has an PI3K-alpha-inhibilory activity of about 0.02 uM or less and an mTOR-inhibitory activity of about 0.02 μM or less. In another embodiment the Compound of the Invention has an PI3K-alpha-inhibiiory activity, of about 0.01 μM or less and an mTOR-inhibitory activity of about 0.01 μM of less.

In another embodiment, Compounds of the invention are also useful as inhibitors of PI3Kα and/or mTOR in vivo for studying the in vivo role of PI3Kα and/or mTOR in biological processes, including the diseases described herein. Accordingly, the invention also comprises a method of inhibiting PI3Kα and/or mTOR in vivo comprising administering a compound or composition of the invention to a mammal.

In another embodiment of any of the embodiments of Embodiment (W), the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.

Another embodiment is directed to a method for idenitying a selective ihhibitor of a PI3K isozyme, the method comprising: (a) contacting a first cell bearing a first mutation in a PI3K-α with a candidate inhibitor; (b) contacting a second cell bearing a wild type P13K-α, a, a PTEN null mutation, or a second mutation in said PI3K-60 with the candidate inhibitor; and (c) measuring AKT phosphorylation in said first and said second cells, wherein decreased AKT phosphorylation in said first cell when compared to said second cell identifies said candidate inhibitor as a selective PI3K-α inhibitor.

As noted above, the newly discovered association between selective genetic mutations and increased sensitivities of some cancers to specific inhibitors renders a particular genetic background more susceptible to one or more types of inhibitors than others. This association between genetic backgrounds and susceptibilities of certain cancers provides an attractive and convenient cellular platform for identification of new selective inhibitors to PI3K kinases (e.g. via screening assays to delect compounds or entities that inhibit phosphorylation in a PI3-αdependent manner). As will be appreciated by those of ordinary skill in the an, any kind of compounds or agents can be tested using the inventive screening methods. A candidate inhibitor compound may be a synthetic or natural compound: it may be a single molecule, a mixture of different molecules or a complex of at least two molecules. A candidate inhibitor can comprise functional groups necessary for structural, interaction with proteins, particularly hydrogen bonding and lipophilic binding, and typically include at least an amine, carbonyl, hydroxyl, ether, or carboxyl group, for example at least two of the functional chemical groups. The candidate inhibitor often comprises cyclical carbon or heterocycloalkyl structures and/or aromatic or heteroaromatic structures substituted with one or more of the above functional groups. Candidate inhibitors are also found among biomoleculcs including peptides, saccharides, fatty acids, steroids, purines, pyrimidines, derivatives, structural analogs, or combinations thereof. In certain embodiments, the inventive methods are used for testing one or more candidate inhibitor compounds. In other embodiments, the inventive methods are used for screening collections or libraries of candidate inhibitor compounds. As used herein, the term “collection” refers to any set of compounds, molecules or agents, while the term “library” refers to any set of compounds, molecules or agents that are structural analogs.

Libraries of candidate inhibitor compounds that can be screened using the methods of the present invention may be either prepared or purchased from a number of companies. Synthetic compound libraries are commercially available from, for example. Comgenex (Princeton, N.J.), Brandon Associates (Merrimack, N.H.), Microsource (New Milford, Conn.), and Aldrich (Milwaukee, Wis.). Libraries of candidate inhibitor compounds have also been developed by and are commercially available from large chemical companies. Additionally, natural collections, synthetically produced libraries and compounds are readily modified through conventional chemical, physical, and biochemical means.

Cells to be used in the practice of the screening methods described herein may be primary cells, secondary cells, or immortalized cells (e.g., established cell lines). They may be prepared by techniques well known in the an (for example, cells may be obtained, by fine needle biopsy from a patient or a healthy donor) or purchased from immunological and microbiological commercial resources (for example, from the American Type Culture Collection (ATCC). Manassas, Va.). Alternatively or additionally, cells may be genetically engineered to contain, for example, a gene of interest. In a first set of cells, the cells possess a genetic mutation in PI3K-α kinase domain, for example, H1047R. In a second set of ceils to be used in the screening assays, the second set of cells possess a genetic mutation in a different kinase catalytic subunit, (for example, a mutation in a helical domain, for example, E545K, or in a different regulatory protein, for example Phosphatase and Tensin Homolog (PTEN). When a candidate inhibitor inhibits phosphorylation, (for example AKT phosphorylation) to a higher degree in the cell possessing the PI3K-α kinase domain genetic mutation when compared to a cell possessing a genetic mutation in a different kinase catalytic subunit, (for example a mutation in a helical domain, for example, E545K, or in a differeni regulatory protein), then the candidate inhibitor is a selective inhibitor for cancers or tumors that harbor activation mutations in PI3K-α. Conversely, P13K-α-selectiye compounds inhibit AKT phosphorylation, PI3K pathway activation, and cell proliferation with greater potency in tumor cells harboring the PI3K-α-H1047R mutation compared to PTEN negative, PI3K-α wild-type, and PI3K-α-E545K backgrounds. Both PTEN inactivalion and KRAS activation desensitize cells to the growth inhibitory effects of PI3K-α-selective compounds. A wild-type PI3K-α is illustratively provided in SEQ ID NO: 1 and is encoded by a mRNA of SEQ ID NO: 2.

In some embodiments, the first and second cells used in the screening assay have different genetic backgrounds. In one embodiment, the first cell group has a genetic mutation in a PI3K-α kinase domain. In an illustrative embodiment, the genetic mutation in the first cell group includes a mutation in a mRNA (GenBank Accession No. NM 006218, version NM 006218.2 GI: 54792081 herein disclosed as SEQ ID NO: 2 which encodes a full length PI3K-α having a mutation in the kinase domain. In one embodiment, an exemplary mutation is at a codon (3296, 3297 and 3298). In the kinase domain of SEQ ID NO: 2, wherein the codon is mutated to provide an amino acid other than a histidine at position 1047 of PI3K-α provided in SEQ ID NO: 1. In one exemplary mutation, the histidine al 1047 is mutated to arginine (H1047R). This mutation has been previously reported to be a particularly oncogenic mutation in the PI3K/AKT signaling pathway. The second cell group lacks the mutation of the first test cell group. In one embodiment, an exemplary mutation is at a codon (1790, 1791 and 1792), in the helical domain of SEQ ID NO: 2, wherein the codon is mutated to provide an amino acid other than a glutamic acid at position 542 or 545 of PI3K-α provided in SEQ ID NO: 1. In one exemplary mutation, the glutamic acid at 545 is mutated to lysine (for example, E542K or E545K). This mutation has also been previously reported to be a particularly oncogenic mutation in the PI3K/AKT signaling pathway.

In some embodiments, the second cell group can harbor a mutation in PTEN.

In some embodiments, the first cell group can include various cell lines, including cancer cell lines, for example breast cancer cell lines that may be commercially available from the American Type Culture Collection ((ATCC) American Type Culture Collection. Manassas, VA.) bearing the H047R het genetic mutation of PI3K-α. In some embodiments, the first cell can include HCT-116, T-47D, MDA-MB-453, SIGOV-3., BT-20 or LS H74T cell lines. In some embodiments, the second cell can include MCF-7, PC3 MCI-H460, SK- BR-3, PC-3, MDA-MB-468, SK-BR-3, MDA-MB-23IT, or A549. Each specific cell line can be maintained according to instructions provided upon purchase and are commonly available, through the ATCC.

In some embodiments, the first cell group and second cell grpup can also include non-tumor cell lines that have been transformed with a mutant PI3K-α catalytic subunit, for example. H1047R het or E545K PI3K-α catalytic subunit. Methods of introducing nucleic acids and vectors into isolated cells and the culture and selection of transformed host cells in vino are known in the art and include the use of calcium chloride-mediated transformation, transduction, conjugation, triparental mating. DEAE, dextran-mediated transfection, infection, membrane fusion with liposomes, high velocity bombardment with DNA-coatcd microprojectilcs, direct microinjection into single cells, and electroporation (see. e.g., Sambrook et al., supra: Davis et al.. Basic Methods in Molecular Biology, 2^{nd} ed., McGraw-Hill Professional, 1995; and Neumann el al., EMBO J., 1:841 (1982)). There are several methods for eukaryotic cell transformation, either transiently or stably using a variety of expression vectors. Methods for mutating a cell-line, for example NIH 3T3 cells by amplifying a sequence of DNA encoding the mutated PI3K-α catalytic subunit of interest. The amplified PGR mutant PI3K-α construct can be cloned into a viral expression vector, for example, pSX2neo, a Moloney murine leukemia virus (MLV) long terminal repeat-driven expression vector made by inserting a simian virus 40 early promoter-neomycin phosphotransferase gene into pSX2, designed to express high leIcvels of 10A1 MLV Env. Transformation of NIH 3T3 cells can be performed by transfection with a different CaPO₄ coprecipitation technique. After reaching confluence the cells can be transferred into a medium containing 5% FBS without dexamethasone, Morphologically transformed cells can be separated and isolated from mixtures of transformed and nontransformed Env-plasmid-transfectcd cells by excising the transformed foci from the cell layer with a small-bore pipette (a Pasteur pipette drawn out over a flame to give a fine tip) and aspiration of the foci by the use of a rubber btilb attached to a pipette.

In some embodiments, the methods described herein require that the cells be tested in the presence of a candidate inhibitor, wherein the candidate inhibitor is added to separate exemplary assay wells, each well containing either the first or second cells. The amount of candidate inhibitor can vary, such that a range of inhibitory activities can be determined for the determination of an IC₅₀ for that candidate inhibitor. This can easily be achieved by serially diluting the compound in an appropriate solvent, for example,.. DMSO and then in the culture medium in which the first and second cells are being incubated in. In some embodiments, the concentration of the candidate inhibitor can range from about 1 μM to about 1 nM concentration. In some embodiments, the candidate inhibitors are added in amounts ranging from about 0.5 nM to about 10 μM. Ihe incubation of candidate inhibitor with first and second cell groups can vary, typically ranging from about 30 minutes to about 60 hours.

In some embodiments, particularly with PI3K-α mediated activity, the cells are stimulated with a growth factor. The selection of growth factor is mediated by the requirements of the cell line, for example, illustrative growth factors can include VEGF, IGF, insulin and hrrrgulin.

In some embodiments, the inhibitory activity of the candidate compounds can be measured using a variety of cellular activities. When cancer cell lines are being used, the inhibition of PI3K mediated activity, e.g., AKT phosphorylation (both at residues S473 and T308); AKT activation, cellular proliferation, and apoptosis resistance in the cells can all be measured. In some embodiments, the amount of AKT phosphorylation in the first and second cell groups can be measured using a phopho-specific antibody (for example AKT1 (phospho S473, Cat. No. ab8932, AKT1 (phospho T308) Cat. No. ab66134) which are commercially available from AbCam. Cambridge, Mass. Other methods for measuring the inhibition of PI3K-α activity in the first and second, cell groups are described in Donahue, A.D. et al., Measuring phosphorylated Akt and other phosphoinositide 3-kinase-regulated phosphoproteins in primary, lymphocytes. Methods Enzymol, 2007(434): 131 -154 which is incorporated herein by reference in its entirety.

In another-embodiment, the invention provides a method for determining a treatment regimen for a cancer patient having a tumor comprising a PI3K-α the method comprising:

determining the presence or absence of a mutation in amino acids 1047 and/or 545 of thePI3K-α;

wherein if the PI3K-α has a mutation at position 10471 the method comprises administering to the cancer patient a therapeutically effective amount of a PI3K-α selective inhibitor compound: or

wherein if the PI3K-α has a mutation at position 545, the method comprises administering to the cancer patient a therapeutically effective amount of a combination of a PI3K-α selective inhibitor and a P13K-β selective inhibitor, a dual PI3K-α/mTOR selective inhibitor, or a combination of a PI3K-α selective inhibitor and a mTOR selective inhibitor.

In another embodiment, the invention provides a method for determining a treatment regimen for a cancer patient havinga tumor comprising a PI3K-α the method comprising:

determining the presence or absence of a mutation in amino acids 1047 and/or 545 of the PI3K-α;

wherein if ihe PI3K-α has a mutation at position 1047, the method comprises administering to the cancer patient a therapeutically effective amount of a PI3K-α selective inhibitor compound, a dual PI3K-α/mTOR selective inhibitor, a combination of a PI3K-α selective inhibitor and a mTOR selective inhibitor, to the subject; or

wherein if the PI3K-α has a mutation at position 545, the method comprises administering to the cancer patient a therapeutically effective aniount of a combination of a PI3K-α selective inhibitor and a PI3K-α selective inhibitor, a dual PI3K-α/mTOR selective inhibitor, or a combination of a PI3K-α selective inhibitor and a mTOR selective inhibitor.

The method of the invention can be used to identify cancer patient populations more likely to benefit from treatment with PI3Kα-selective inhibitors as well as patient populations less likely to benefit.

The invention can be used to further define genetic markers or gene expression signatures which identify PI3Kα inhibitor sensitive tumor subtypes by extended in vitro cell line profiling and in vivo pharmacodynamic and efficacy studies.

In some embodiments, a method for determining a treatment regimen for a cancer patient having the exemplified cancers herein caiin be readily performed on the basis of the differential activity of PI3K-α selective inhibitors in cancers having a PI3K-α mutated background described herein. In patients in which a tumor cell has been analyzed and assayed to determine whether the tumor harbors a PI3Kα mutation in the kinase domain, for example, a mutation resulting in H1047R, greater efficacy and treatment improvement can be achieved by tailorings treatment comprising a PI3K-α selective inhibitor. For patients, who have a tumor which does not harbor a mutation in PI3Kα kinase domain, the treatment may require adopting a different treatment regimen, for example, by focusing on delivery of a combination of PI3K-α selective inhibitors and a PI3K-β selective inhibitor, a dual PI3K-α/mTOR selective inhibitor, or a combination of a PI3K-α selective inhibitor and a mTOR selective inhibitor. As indicated above, the PI3K-α selective inhibitors, mTOR selective inhibitors and dual PI3K-α/mTOR selective inhibitors are exemplified in Tables 1, or 2, or 3, and in the detailed description herein.

In some embodiments, methods for determining a treatment regimen comprises determining the presence of a mutation in amino acids 1047 and/or 545 of the PI3K-α in the subject's tumor. This step can be achieved in a variety of ways, using nucleic acid approaches, protein separation approaches or direct immunological approaches using mutation specific antibodies, in some embodiments, presence of a mutation in amino acids 1047 and/or 545 of the PI3K-α in the subject's tumor can be determined using any suitable method for the sequence analysis of amino acids. Examples of suitable techniques include, but are not limited to, western-blot analysis, immunoprecipitation, radioimmunoassay (RIA) or enzyme-linked imnumoabsorbent assay (ELISA).

In the present invention, reference to position within the amino acid sequence of PI3Kα is made referring to SEQ ID NO: 1. Reference to positions within the nucleotide sequence of ihe P13Kα is made referring to SEQ ID NO:2. Specific amino acids in the wild type protein sequence are described using single letter amino acid designation followed by the position in ihe protein sequence, for example E545 indicates that position 545 is glutamic acid. To represent a substitution at a particular position, the substituted amino acid follows the position, for example E545K indicates that the glutamic acid at position 545 is replaced with a lysine.

Determining the presence or absence of mutations in the sequence of the PI3 K-α peptide sequence is generally determined using in vitro methods wherein a tumor sample is used which has been removed from the body of a patient.

Determining the presence or absence of imitations in the amino acid sequence of PI3Kα or a portion thereof, can be done using any suitable method. For example the nucleotide sequence of P13Kaα or a portion thereof maybe determined and the amino acid sequence deduced from the nucleotide sequence or a PI3K-α protein can be interrogated directly.

The nucleotide sequence of the PI3K-α, or a portion thereof, may be determined using any method for the sequence analysis of nucleic acids. Methods for identification of sequence mutation in genes are well known in the art and the mutations in the PI3Kα can be identified by any suitable method. These methods include, but are not limited to, dynamic allele-specific hybridization; the use of molecular beacons: enzyme-based methods, using for example DNA ligase, DNA polymerase or nucleases; PCR based methods, whole genome sequencing; partial genome sequencing; exome sequencing; nucleic acid probe hybridization; and restriction enzyme digestion analysis.

Methods of Direct DNA sequencing are well known in the art, ( see for example: Current Protocols in Molecular Biology, edited by FRed M. Ausubel, Roger Brent, RobertE, Kingston, David D. Moore, J. G. Seidman, John A. Smith, Kevin Struhl, and Molecular Cloning: A Laboratory Manual, Joe Sambrook, David W Russel, 3^{rd} edition. Cold Spring Harbor Laboratory Press).) These sequencing protocols include for example, the use of radioactively labeled nucleotides, and nucleotides labeled with a fluorescent dye.

For example, Barbi, S. et al., used the following protocol to sequence the helical domain (exoti 9) and the kinase domain (exon 20) of PI3Kα3. Normal and tumor DNA was extracted from paraffin-embedded tissue, and amplified using fluorescent dye-labeled primers. Primer sequences need be chosen to uniquely select for a for a region of DNA, avoiding the possibility of mishybridization to a similar sequence nearby. A commonly used method is BLAST search whereby all the possible regions to which a primer may bind can be seen. Both the nucleotide sequence as well as the primer itself can be BLAST searched. The free NCBI tool Primer-BLAST integrates primer design tool and BLAST search into one application, so does commercial software product such as Beacon Designer, (Premier Biosoft International, Palo Alto Calif.). Mononucleotide repeats should be avoided, as loop formation can occur and contribute to mishybridization. In addition, computer programs are readily available to aid in design of suitable primers. In certain embodiments the nucleic acid probe is labeled for use in a Southern hybridization assay. The nucleicacid prbe may be radioaetively labeled, fluorescently labeled or is immunologically detectable, in particular is a digoxygenin-labeled (Roche Diagnostics GmbH, Mannheim).

In some embodiments, determining the presence of a helical domain mutation in exon 9 can include the use of forward primer and reverse primers: GGGAAAAATATGACAAAGA A AGC (SEQ ID NO: 3) and CTGAGATCAGCCAAATTCAGTT (SEQ ID NO: 4) respectively and a sequencing priMer can include TAGCTAGAGACAATGAATTAAGGGAAA (SEQ ID NO: 5).

For determining a mutation in the kinase domain iN exon 20, an exemplary set Of primers can include: forward and reverse primers CTCAATGATGCTTGGCTCTG(SEQ ID NO: 6) and TGGAATCCAGAGTGAGCTITC (SEQ ID NO: 7) respectively and the sequencing primer can include TITGATGACATTGCATACATTCG (SEQ ID NO: 8). The amplification products can then be sequenced. (Barbi, S. et al. J. Experimental and Clinical Cancer Research 2010, 29:32) The sequences are then compared and differences between the wild type PI3Kα sequence and the sequence of the tumor PI3Kα are determined. The assay could also be performed by only amplifying the tumor DNA and comparing the PI3K-α sequence in the tumor with the sequence of SEQ ID NO: 1.

In some embodiments, the present invention provides polynucleotide sequences comprising polynucleotide sequences in whole or in part from SEQ ID NO: 2 that are capable of hybridizing to the helical region, or the kinase domain of PI3K-α under conditions of high stringency. In some embodiments, the polynucleotides can include sequences complementary to nucleic acid sequences that encode in whole or in part PI3K-α or PI3K-α having specific mutations as described herein. The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands lias significant effects on the efficiency and strength of hybridization between nucleic acid strands. This is of particular importance in amplification reactions, as well as detection methods which depend upon binding between nucleic acids.

In some embodiments, the present invention provides polynucleotide sequences comprising polynucleotide sequences in whole or in part from SEQ ID NO: 2 that are capable of hybridizing to the helical region, or live kinase domain oPI3K-α under conditions of high stringency. In some embodiments, the present method includes using isolated RNA from a subject's tumor in an assay to determine whether there is a mutation at amino acid at position 1047, 542, or 545 of SEQ ID NO: 1, the assay further comprises: (a) reverse transcribing said RNA sample into an equivalent cDNA: (b) amplifying a predetermined region of the cDNA using a pair of nucleic acid probes directed to a predetermined region of the PI3K-α gene: (c) sequencing said amplified cDNA region to obtain a polynucleotide sequence of said amplified cDNA region; and (d) determining whether said amplified cDNA region contains a gene mutation in a codon encoding the amino acid at position 1047, 542, or 545 of SEQ ID NO:1.

In some embodiments, the present methods can employ amplifying a predetermined region of the cDNA by amplifying the cDNA using a pair of nucleic acid primers, a first primer capable of hybridizing stringently to the cDNA upstream of a DNA codon encoding the amino acid at cither amino acid 1047 or 542 or 545 of SEQ ID NO: 1, and second a nucleic acid primer operable to hybridize stringently to the cDNA downstream of a DNA codon encoding the amino acid at either amino acid 1047 or 542 or 545 of SEQ ID NO:1

In some embodiments, the polynucleotides can include sequences complementary to nucleic acid sequences that encode in whole or in part PI3K-α or PI3Kα having specific mutations as described herein. The terms “complementary” and “complementarity” refer to polynucleotides (i.e., a sequence of nucleotides) related by the base-pairing rules. For example, for the sequence “A-G-T,” is complementary, to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

“High stringency conditions” when used in reference to nucleic acid hybridization comprise conditions equivalent to binding or hybridization at 42° C. in a solution consisting of 5x SSPE (43.8 g/l NaCl, 6.9 g/l NaH₂PO_{4.}H₂O and 1.85 g/l EDTA, pH adjusted to 7.4 with NaOH), 0.5% SDS, 5x Denhardt's reagent and 100 μg/mL denatured salmon sperm DNA followed by washing ina solution comprising 0.1 x SSPE, 1.0% SDS al 42° C. when a probe of about 500 nucleotides in length is employed.

The term “homology” when used in relalion to nucleic acids refers to a degree of complementarity. There may be partial homology or complete homology (i.e., identity). “Sequence identity” refers to a measure of relaieclness between two or more nucleic acids or proteins, and is given as a percentage with reference to the total comparison length. The identity calculation lakes into account those nucleotide or amino acid residues that are identical and in ihe same relative positions in their respective larger sequences.nCalculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using ihe functional term “substantially homologous.” The inhibition of hybridization of the completely complementary sequence, to the target sequence may be examined using a hybridization assay (Southern or Northern blot, solution hybridization and the like) under conditions of low stringency. A substantially homologous sequence or probe will compete for and inhibit ihe binding (i.e., the hybridization) of a sequence which is completely homologous to a target under conditions of low-stringency. This is not lo say that conditions of low stringency are such that non-specific binding is permitted; low stringency conditions require thai the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize lo the second non-complementary target.

In preferred embodiments, hybridization-conditions are based on the melting temperature (Tm) of the nucleic acid binding complex and confer a defined “stringency” The term “hybridization” refers to the pairing of complementary nucleic acids. Hybridization and the strength of hybridization (i.e., the strength of the association between nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

The term “Tm” refers to the “melting temperature” of a nucleic acid. The melting temperature is the temperature at which a population of double-stranded nucleic acid molecules becomes half dissociated into single strands. The equation for calculating the Tm of nucleic acids is well known in the art. As indicated by standard references, a simpie estimate of the Tmvalue may be calculated by the equation: Tm=81.5+0.41(% G+G), when a nucleic acid is in aqueous solution at 1 M NaCl. The term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences.

In addition, sequence mutations in the PI3Kα can be determined using any sequence-specific nuclcic acid detection method allowing delection of single-nucleotide, variation, in particular any such method involving complementary base pairing. For example, to determine if the PI3K-α comprises a E545 mutation, the sequence of P13K-α peptide or a portion thereof comprising nucleotides 1790, 1791 and 1792 of SEQ ID NO:2 (codon corresponding with position 545 in the amino acid sequence), is used in a polymerase chain reaction (PGR) where the oligonucleotide primers allow ihe amplification of PI3Kα only if the nucleotide at position 1790 is G. If no reaction product is formed then the amino acid at position 545 is mutated. In another example the oligonucleotide primers are designed to allow the amplification of the to allow amplification if the nucleotide at position 3297 is A (codon comprising nucleotides 3296, 3297 and 3298 corresponds with position-1047 of the amino acid sequence). If no reaction product is formed using, those, primers then the amino, acid at position 545 is mutated.. Methods for performing PGR are known in the art {see Current Protocols in Molecular Biology, edited by Fred M. Ausubel, Roger Brent, Robert E. Kingston, David D, Moore, J. G. Seidman, John A. Smith, Kevin Struhl, and Molecular Cloning:. A Laboratory Manual. Joe Sambrook. David W Russel. 3³⁴ edition. Cold Spring Harbor Laboratory Press).

Dynamic allele-specific hybridization (DASH) genotyping takes advants of the differences in the melting temperature in DNA that results from the instability of mismatched base pairs. This technique is well suited to automation. In the first step, a DNA segment is amplified and attached to a bead through a PCR reaction with a biotinylated primer. In the second step, the amplified product is attached to a streptavidin column and washed with NaOH to remove the un-biotinylated strand. An sequence-specific oligonucleotide is then added in the presence of a molecule that fluoresces when bound to double-stranded DNA. The intensity is then measured as temperature is increased until the Tm can be determined. A single nucleotide change will result in a lower than expected Tm (Howell W., Jobs M., Gyllensten U., Brookes A. (1999) Dynamic allele-specific hybridization. A new method for scoring single nucleotide polymorphisms: Nat Biotechmol, 17(1):87-8). Because DASH genolyping is measuring a quantifiable change in Tm, it is capable of measuring all types of mutations, not just SNPs. Other benefits of DASH include its ability to work with label, free probes and its simple design and performance conditions.

Molecular beacons can also be used to detect mutations in a DNA sequences Molecular beacons makes use of a specifically engineered single-stranded oligonucleotide probe. The oligonucleotide is designed such that there are complementary regions at each end and a probe sequence located in between. This design allows, the probe to lake on a hairpin, or stem-loop, structure in its natural, isolated state. Attached to one end of the probe is a fluorophore and to the other end a fluorescence-quencher. Because of the stem-loop structure of the probe, the fluorophore is in close proximity to the quencher, thus preventing the molecule from emitting any fluorescence. The molecule is also engineered such that only the probe sequence is complementary to the to the genomic DNA that will be used in the assay (Abravaya K., Huff J., Marshall R., Merchant B., Mullen C., Schneider G., and Robinson J. (2003) Molecular beacons as diagnostic tools: technology and applications. Clin Client Lab Med., 41: 468-474). If the probe sequence of the molecular beacon encounters its target genomic DNA during the assay, it will anneal and hybridize, Because of the length of the probe sequence, the hairpin segment of the probe will denatured in favor of forming a longer, more stable probe-target hybrid. This conformational change permits the fluorophore and quencher to be free of their tight proximity due to the hairpin association, allowing the molecule to fluoresce. If on the other hand, the probe sequence encounters a target sequence with as little as one non-complementary nucleotide, the molecular beacon will preferentially stay in its natural hairpin state and no fluorescence will be observed, as the fluorophore remains quenched. The unique design of these molecular beacons allows for a simple diagnostic assay to identify SNPs at a given location. If a molecular beacon is designed to match a wild-type allele and another to match a mutant of the allele, the two can be used to identify the genotype of an individual. If only the first probe's fluorophore wavelength is detected during the assay then the individual is homozygous to the wild type. If only the second probe's wavelength is detected then the individual is homozygous to the mutant allele. Finally, if both wavelengths are detected, then both molecular beacons must be hybridizing to their complements and thus the individual must contain both alleles and be heterozygous.

Enzyme-based nucleic acid methods are also suitable and contemplated for determining mutations in the PI3K-α nucleotide sequence. For example, Restriction fragment length polymorphism (RFLP) (discussed in greater detail below) can be used to detect single nucleotide differences. SNP-RFLP makes use of the many different restriction endonuclease and their high affinity to unique and specific restriction sites. By performing a digestion on a genomic sample and determining fragment, lengths through a gel assay it is possible to ascertain whether or not the enzymes cut the expected restriction sites. A failure to cut the genomic sample results in an identifiably larger than expected fragment implying that there is a mutation at the point of the restriction site which is rendering it protected from nuclease activity.

The term “functionally equivalent codon” is used herein to refer to codons that encode the same amino acid, such as the six codons for arginine.

In one embodiment of the invention the method comprises at least one nucleic acid probe or oligonucleotide for determining the sequence of the codon that encodes amino acid 1047. In another embodiment the method comprises at least one, nucleic acid probe or oligonucleotide for determining ihe sequence of the codon dial encodes amino acid 545. The oligonucleotide is a PCR primer, preferably a set of PGR primers which allows amplification of a PI3Kα nucleic acid sequence fragment only if the codon which encodes amino acid 1047 encodes a histidine. In another method, the PCR primer or set of PCR primers allows the amplification of nucleic acid sequence fragment only if the codon which encodes amino acid 545 encodes a glutamic acid. Determination of suitable PGR primers is routine in the art. (Current Protocols in Molecular Biology, edited by Fred M. Ausubel, Roger Brent, Robert E. Kingston, David D. Moore, J. G. Seidman, John A. Smith, Kevin Struhl; Looseleaf: 0-471-650338-X: CD-ROM: 0-471 -30661-4). In addition, computer programs are readily available to aid in design of suitable primers. In certain embodiments the nucleic acid probe is labeled for use in a Southern hybridization assay. The nucleic acid probe may be radioactively labeled, fluorescently labeled or is immunologically detectable, in particular is a digoxygenin-labcled (Roche Diagnostics GmbH, Mannheim).

U.S. Patent Publication 20010016323 discloses methods for detecting point mutations using a fluorescently labeled oligonucleotidemeric probe and fluorescence resonance energy transfer. A point mutation leading to a base mismatch between the probe and the target DNA strand causes the melting temperature of the complex to be lower than the melting temperature for the probe and the target if the probe and target were perfectly matched.

Other suitable methods for detecting single point mutations include those disclosed in for example. U.S. Patent Publication 2002010665, which involves the use of oligonucleotide probes in array formal. Such arrays can include one or more of SEQ ID NOs:3-8, U.S. Patent Publication 20020177157 discloses additional methods for delecting point mutations.

A polynucleotide carrying a point mutation leading to a mutation of PI3K-α kinase domain, for example, H11047R that is the subject of this invention can be identified using one or more of a number of available techniques. However, detection is not limited to the techniques described herein and the methods and compositions of the invention are not limited to these methods, which are provided for exemplary purposes only. Polynucleotide and oligonucleotide probes are also disclosed herein and are within the scope of the invention, and these probes are suitable for one or more of the techniques described below. These include allele-specilic oligonucleotide hybridization (ASO), which, in one embodiment, is a diagnostic mutation detection method wherein hybridization with a pair of oligonucleotides corresponding to alleles of a known mutation is used to detect the mutation. Another suitable method is denaturing high performance liquid chromatography (DHPLC), which is a liquid chromatography method designed to identify mutations and polymorphisms based on detection of heteroduplcx formation between mismatched nucleotides. Under specified conditions, heleroduplexes eltite from the column earlier than homoduplexes because of reduced melting temperature. Analysis can then be performed on individual samples.

An amplified region of the DNA containing the mutation or the wild-type sequence can be analyzed by DHPLC. Use of DHPLC is described in U.S. Pat. Nos. 5,795,976 and 6,453,244, both of which are incorporated herein by reference. A suitable method is that provided by Transgenomic, Inc. (Omaha. Nebr.) using the Transgenomic WAVE® System.

For ASO, a region of genomic DNA or cDNA containing the PI3K-α mutation (H1047R aiid/or E545K) is amplified by PCR and transferred onto duplicating membranes. This can be performed by dot/slot blotting, spotting by hand, or digestion and Southern blotting. The membranes are prehybridized, then hybridized with a radiolabeled of dcoxygenin (DIG) labeled oligonucleotide to cither the mutant or wild-type sequences. For the DIG lahel, detection is performed using chemiluminescent or colorimetric methods. The membranes are then washed with increasing stringency until the ASO is washed from the non-specific sequence. Following autoradiographic exposure, the products are scored for the level of hybridization lo each oligonucleotide. Optimally, controls are included for the normal and mutant sequence on each filter to confirm correct stringency, and a negative PCR control is used to check for contamination in the PCR.

The size of the ASO probe is not limited except by technical parameters of the art. Generally, too short a probe will not be unique to the location, and too long a probe may cause loss of sensitivity. The oligonucleotides are preferably 15-21 nucleotides in length, with the mismatch twoards the center of the oligonucleotide.

The region of sample DNA on which ASO hybridization is performed to detect the mutation of this invention is preferably amplified by PCR using a forward primer. For exon 9 the forward primer and reverse primers were GGGAAAAATATGACAAAGAAAGC (SEQ ID NO: 3) and CTGAGATCAGCCAAATTCAGTT (SEQ ID NO: 4) respectively and the sequencing primer was TAGCTAGAGACAATGAATTAAGGGAAA (SEQ ID NO: 5). for exon 20 the forward and reverse primers were CTCAATGATGCTTGGCTCTG (SEQ ID NO: 6) and TGGAATCCAGAGTGAGCTTTC (SEQ ID NO: 7) respectively. In this case, amplification by PCR or a comparable method is not necessary but can oplionally be performed.

Optionally, one or more than one of the amplified regions described above, (including the 306 nucleotide region generated using primers of SEQ ID NO:3-8, or shorter portions of either of these regions, can be analyzed by sequencing in order to detect the mutation. Sequencing can be performed as is routine in the art. The only limitation on choice of the region to be sequenced, in order to identify the presence of the mutation, is that the region selected for sequencing must include the nucleotide that is the subject of the mutation. The size of the region selected for sequencing is not limited excepi by technical parameters as is known in the art, and longer regions comprising part or all of the DNA or RNA between selected amplified regions using ihe.primers SEQ ID NOs: 3 & 4 and 6 & 7 disclosed herein can be sequenced.

Variations of the methods disclosed above are also suitable for detecting the mutation. For example, in a variation of ASO, the ASO's are given homopolymer tails with terminal deboyribonucleotidyl transferase, spotted onto nylon membrane, and covalently bound by UV irradiation. The target DNA is amplified with biotinylated primers and hybridized to the membrane containing the immobilized oligonucleotides, followed by detection. An example of this reverse doi blot technique is ihe INNO-LIPA kit from tnnogerietics ( Belgium).

With the identification and sequencing of the mutated gene and the gene product, i.e. SEQ ID NO:1 having a mutation at E545K and H1047R, probes and antibodies raised to the gene product can be used in a variety of hybridization and immunological assays to screen for and detect the presence of either a normal or mutated gene or gene product.

Expression of the mutated gene in heterologous cell systems can be used to demonstrate structure function relationships. Ligating the DNA sequence into a plasmid expression vector to transfect cells is a useful method to test the influence of the mutation on various cellular biochemical parameters. Plasmid expression vectors containing either the entire normal or mutant human or mouse sequence or portions thereof, can be used in in vitro mutagenesis experiments which will identify portions of the protein crucial for regulatory function.

The DNA sequence can be manipulated in studies lo understand the expression of the gene and its product, and to achieve production of large quantities of the protein for functional analysis, for antibody production, and for patient therapy. Changes in the sequence may or may not alter ihe expression pattern in terms of relative quantities, tissue-specificity and functional properties.

A number of methods are available for analysis of variant (e.g., mutant or polymorphic) nucleic acid sequences. Assays for detections polymorphisms or mutations fall into several categories, including, but not limited to direct sequencing assays, fragment polymorphism assays, hybridization assays, and computer based data analysis. Protocols and commercially available kits or services for performing multiple variations of these assays are commercially available and known to those of skill in the art. In some embodiments, assays are performed in combination or in combined pans (e.g., different reagents or technologies from several assays are combined toyicld one assay). The following illustrative assays may be used to screen and identify nucleic acid molecules containing the mutations of PI3K-α mutation of interest.

### Fragment Length Polymorphism Assays

In some embodiments of the present invention, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE 1 [Third Wave Technologies. Madison, Wis.] enzyme). DNA fragments from a sample containing a SNP or a mutation will have a different banding pattern than wild type.

### PGR Assays

in some embodiments of the present invention, variant sequences are detected using a PCR-based assay. In some embodiments, the PCR assay comprises the use of oligonucleotide nucleic acid primers that hybridize only lo the variant or wild type allele of PI3Kα (e.g., to the region of mutation or multiple mutations). Both sets of primers are used to amplify a sample of DNA. If only the mutant primers result in a PGR product, then the subject's tumor or cancer expresses a somatic mutation in an PI3K-α mutation allele. PCR amplification conditions are tailored to the specific oligonucleotide primers or oligonucleotide probes used, the quality and type of DNA or RNA being screened, and other well known variables that can be controlled using appropriate reagents and/or PCR cycling conditions known to those of ordinary skill in the art.

### RFLP Assays

In some embodiments of the present invention, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

### Direct Sequencing Assays

In some embodiments of the present invention, variant sequences are detected using a direct sequencing technique. In these assays, DNA samples are first, isolated from a subject using any suitable method. In some embodiments, the region of interest is cloned into a suitable vector-and amplified bygrowtli in aJiost cell (e.g., a bacteria). In other embodiments, DNA in the region of interest is amplified using PCR.

Following amplification, DNA in the region of interest (e.g., the region containing the SNP or mutation of interest) is sequenced using any suitable method, including but not limited to manual sequencing using radioactive marker nucleotides, or automated sequencing. The results of the sequencing are displayed using any suitable method. The sequence is examined and the presence or absence of a given SNP or mutation is determined.

### CFLP Assays.

In other embodiments, variant sequences are detected using a CLEAVASE fragment length polymorphism assay (CFLP; Third Wave Technologies, Madison, Wis.; See e.g.. U.S. Pat. Nos. 5,843,654; 5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific; thermostable nuclease that recognizes and cleaves the junctions between these single-stranded-and double-stranded regions. The region of interest is first isolated, for example, using PCR. Then, DNA strands are separated by healing. Next, the reactions are cooled to allow intra-strand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### Hybridization Assays

In some embodimenls of thee preseni invention, variant sequences are detected by hybridization analysis in a hybridization assay. In a hybridization assay, the presence or absence of a given mutation is determined based on the ability of the DNA from the sample to hybridize to a complementary DNA molecule (e.g., a oligonucleotide probe or probes as illustrated herein). A variety of hybridization assays using a variety of technologies for hybridization and detection are available. Relevant and useful hybridization assays for practicing the methods of the preseni invention are provided below.

### Direct Detection of Hybridization

In some embodiments,hybridization of a probe to the sequence of interest (e.g., a SNP or mutation) is detected directly by visualizing a bound probe (e.g., a Northern or Southern assay; See e.g., Ausabel et al. (eds.) (1991) Current Protocols Molecular Biology, John Wiley & Sons, NY). In a these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g.. on an agarose gel) and transferred to a membrane. A labeled (e.g.. by incorporating a radionucleotide) probe or probes specific for the SNP or mutation being detected is allowed to contact the membrane under a condition or low, medium, or high stringency conditions. The unbound probe is removed and the presence of binding is detected by visualizing the labeled probe.

### Detection of Hybridization Using “DNA Chip” Assays

In some embodiments of the present invention, variant sequences are detected using a DNA chip hybridization assay. In this assay, a series of oligonucleotide probes are affixed to a solid support. The oligonucleotide probes are designed to be unique to a given SNP or mutation. The DNA sample of interest is contacted with the DNA “chip” and hybridization is detected.

In some embodiments, an illustrative and commercially available DNA chip assay can include a GENECHIP® (commercially available from Affymetrix, Santa Clara, Calif. USA); See e.g., U.S. Pat. Nos. 6,045,996:5,925,525; and 5,858,659; each of which is herein incorporated by reference) assay. The GENECHIP(c)® technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in ihe semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent rcportcrgroup. The labeled DNA is then incubated with the array, using a fluidics station. The array is then inserted into the scanner, where patterns, of hybridization are detected. The hybridization data are collected as light emitted from the fluorescent reporter groups already incorporated into the target, which is bound to the probe array. Probes that perfectly match the large generally produce stronger signals than those that have mismatches. Since the sequence and position of each probe on the array are known, by complementarity, the identity of the target nucleic acid applied to the probe array can be determined.

### Enzymatic Detection of Hybridization

In some embodiments of the present invention, hybridization can be detected by enzymatic cleavage of specific structures (INVADER assay, Third Wave Technologies; See e.g., U.S. Pat. Nos. 5,846,717, 6,090,543; 6,001,567; 5,985,557; and 5,994,069; each of which is herein incorporated by reference). The INVADER assay detects specific DNA and RNA sequences by using structure-specific enzymes to cleave a complex formed by the hybridization of overlapping oligonucleotide probes. Elevated temperature and an excess of one of ihc probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader. The INVADER assay detects specific mutations in unamplified genomic DNA. The isolated DNA sample is contacted with the first probe specific either for a mutation of Ihe preseni invention or wild type PI3K-α sequence and allowed to hybridize. Then a secondary probe, specific to the first probe, and containing the fluorescein label, is hybridized and the enzyme is added. Binding is detected by using a fluorescent plate reader and comparing the signal of ihc lest sample to known positive and negative controls.

In some embodiments, hybridization of a bound probe is detected using a TaqMan assay (PE Biosystems, Foster City, Calif.: See e.g., U.S. Pat. Nos. 5,962,233 and 5,538,848. each of which is herein incorporated by reference). The assay is performed during a PCR reaction. The TaqMan assay, exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-qucneher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorometer.

In accordance with the present invention, diagnostic kits are also provided which will include the reagents necessary, for the above-described diagnostic screens. For example, kits may be provided which include oligonucleotide probes or PGR primers are present for the detcetion and/or amplification of mutant PI3K-α and comparable wild-type PI3K-α -related nucleotide sequences. Again, such probes may be labeled for easier detection of specific hybridization. As appropriate to the various diagnostic embodiments described above, the oligonucleotide probes in such kits may be immobilized to substrates and appropriate controls may be provided. Examples of such oligonucleotide probes include oligonucleotides comprising or consistingof at. feast one of SEQ ID NOs:3&4 and 6&7.

Determining the presence ofabsence of imitations in the amnio acid-sequence of PI3Kα can be determined using any method for the sequence analysis of amino acids. Non-limiting examples include: western blot analysis or ELISA assays, or direct protein sequencing of the PI3Kα in the subject's tumor, in some embodiments, particularly useful antibodies have selectivity for wild type PI3K-α versus the mutant PI3Kα for example, an antibody useful in the assay would bind to wild type PI3K-α or a portion wild type PI3Kα but not to a PI3Kα having a mutation at the amino acid of interest. Particularly useful antibodies could include antibodies which bind the wild type PI3K α which has histidinen, position 1047 but does not bind a mutant PI3Kα which has an amino acid other than histidine, such as arginine, in other words the antibody specifically bind to an epitope comprising histidine at position 1047 . Likewise, particularly useful are antibodies which bind the wild type PI3Kα which has glutamic acid at position 545 but does not bind a mutant PI3Kα which has an amino acid other than glutamic acid at position 545, such as lysine at that position.

Another embodiment of the inveniion provides a method comprising the use of at least one antibody which binds selectively to the wild type. PI3α protein as compared with binding to a mutated form of PI3Kα. Alternately the antibody binds selectively to a mutated form of PI3Kα as compared with binding to the wild type PI3Kα protein and can differentiate between wild-type PI3Kα and PI3Kα-H047R or between wild-type PI3Kα and PI3Kα-E545K. Methods for isolating suitable amounts of target protein from a complex mixture in relatively small amounts (less than 1 mg) are commonly known by those skilled in the art. In one illustrative embodiment, a tumor cell or plurality of tumor cells from a subject's tumor or cancer are lysed using commonly available lysing reagents in the presence of protease inhibitors. The lysate is cleared and the supernatant is either electrophoresed and subjected to a Western Blot using mutation specific antibodies, or alternatively, the mutated PI3Kα-H1047R or PI3Kα-E545K are selectively immunoprecipitated and further dissociated from the capture antibody and subjected to Western Blotting or protein sequenced directly.

“Antibody” includes, any immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′)₂ and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antihody can be of any the five major classes of immunoglobulins: IgA, IgD, IgE, IgG, and IgM, or subclasses (isotypes) thereof (e.g. IgGI, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunii structures and three-dimensional configurations. Antibodies can be naked or conjugated to other molecules such as toxins, radioisotopes and the like.

“Antibody fragment” can refer to a portion of an intact antibody. Examples of antibody fragments include, but are not limited to, linear antibodies; single-chain antibody molecules; Fc or Fe peptides, Fab and Fab, fragments, and multispecific antibodies formed from antibody fragments.

“Chimeric antibodies” refers to antibodies wherein ihe amino acid sequcnce of the immunoglobulin molecule is derived from iwo or mote species. Typically, the variable region of both light and heavy chains corresponds to the variable region of antibodies derived from one species ofmammals (e.g. mouse, rat, rabbit, etc) with the desired specificity, affinity, and capability while the constant regions are homologous to the sequences in antibodies derived from another (usually human) to avoid eliciting an immune response in that species.

“Humanized” form of non-human (e.g., rabbit) antibodies include chimeric antibodies that contain minimal sequence, or no sequence, derived from non-human immunoglobulin. For the most part, humanized antibodies are/human immunoglobulins (recipient antibody) in which residues from a hypei variable region of the recipient are replaced by residues from a hypervariable region of a non-human species (donor antibody) such.as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues thai are not found in the recipient antibody or in the donor antibody. Most often, the humanized antibody can comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically dial of a human immunoglobulin. Methods used to generate humanized antibodies are well known in the field of immunology and molecular biology.

“Hybrid antibodies” can include immunoglobulin molecules in which pairs of heavy and light chains from antibodies with different antigenic determinant regions are assembled together so that two different epitopes or two different antigens can be recognized and bound by the resulting tetramer.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes al least 3-5 and-more usually, at least 5 or 8-10 amino acids in a unique spatial conformation.

“Specifically binds” to or shows “specific binding” twoards an epitope means thai the antibody reacts or associates more frequently, and/or more rapidly, and/or greater duration, and/or with greater affinity with the epitope than with alternative substances.

### Preparat ion of Antibodies

### Polyclonal Antibodies

Polyclonal antibodies are preferahly raised in animals by multiple subcutaneous (sc) or intraperitoneal (ip) injections of the relevant antigen and an adjuvant. Alternatively, antigen may be injected directly into the animal's lymph node (see Kilpatrick et al., Hybridomia, 16:381-389, 1997). An improved antibody response may be obtained by conjugating the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride or other agents known in the art.

Animals are immunized against the antigen, immunogenic conjugates or derivatives by combining, e.g., 100 μg of the protein or conjugate (for mice) with 3 volumes, of Freuud's complete adjuvant and injecting the solution intradermally at multiple sites. One month later, the animals are boosted with 1/5 to 1/10 the original amount of peptide or conjugate in Freuud's complete adjuvant by subcutaneous injection al multiple sites. At 7-14 days post-booster injection, the animals are bled and the serum is assayed for antibody titer. Animals are boosted until the titer plateaus. Preferably, the animal is boosted with the conjugate of the same antigen, but conjugated through a different cross-linking reagent. Conjugates also can be made in recombinant cell culture as protein fusions. Also, aggregating agents such as alum are suitably used to enhance the immune response.

### Monoclonal Antibodies

Monoclonal antibodies can be made using the hybridoma method first described by Kohler et al., Nature, 256:495 (1975)., or by recombinant DNA methods. In the hybridoma method, a mouse or other appropriate host animal, such as rats, hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hyhridomu cell (Coding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells, for example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells and are sensitive to a medium. Human myeloma and mouse-human heteromyeloma cell lines also have been described for the production, of human monoclonal antibodies (Kozbor. J, Immunol., 133: 3001 (1984); Brodeur et al., Monoclonal Antibody Production Techniques and Applications, pp. 51-63 (Marcel Dekkter, Inc., New York, 1987)): Exemplary murine myeloma lines include those derived from MOP-21 and M. C.-11mouse tumors available from the Salk Institute Cell Distribution Center, San Diego, Calif. USA. and SP-2 or X63-Ag8-653 cells available from the American Type Culture Collection. Rockvillc, Md. USA. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding-assay, such as radioimmunoassay (RIA) or enzyme-linked immunoabsorbent assay (ELISA). The binding affinity of the monoclonal antibody can be determined, for example, by BIAcore or Scatchard analysis (Munson et al., Anal. Biochem., 107:220 (1980)).

After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture, media for this purpose include, for example. D-MEMO or RPMI 1640 medium. In addition, the hybridoma cells can be grown in vivo as ascites tumors in an animal. The monoclonal antibodies secreted by the subclones are suitably separated from the cullure medium, ascites fluid, or serum by conventional immunoglobulin purification procedures such as protein A-Sepharose, hydroxylapatite chromatography, gel electrophoresis, dialysis, or affinity chromatography.

### Recombinant Production of Antibodies

The amino acid sequence of an immunoglobulin of interest can be determined by direct protein sequencing, and suitable encoding nucleotide sequences can be designed according to a universal codon table.

Alternatively, DNA encoding the monoclonal antibodies can be isolated and sequenced from the hybridoma cells using conventional procedures (e.g., by using oligonucleotide probes that are capable of binding specifically to genes encoding the heavy and light chains of the monoclonal antibodies). Sequence determination will generally require isolation of at least a portion of the gene or cDNA of interest. Usually this requires cloning the DNA of mRNA encoding the monoclonal antibodies. Cloning is carried out using standard techniques (see, e.g.,Sambrook et al. (1989) Molecular Cloning: A Laboratory Guide, Vols 1-3, Cold Spring Harbor Press, which is incorporated herein by reference). For example, a cDNA library can be constructed by reverse transcription of polyA+ mRNA, preferably membrane-associated mRNA, and the library screened using probes specific for human immunoglobulin polypeptide gene sequences. In a preferred embodiment, the polymerase chain reaction (PCR) is used to amplify cDNAs (or portions of full-length cDNAs) encoding an immunoglobulin gene segment of interest (e.g., a light chain variable segment). The amplified sequences can be cloned readily into any suitable vector, e.g., expression vectors, minigene vectors, or phage display vectors. It will be appreciated that the particular method of cloning used is not critical, so long as it is possible to determine the sequence of some portion of the immunoglobulin polypeptide of interest.

One source for RNA used for cloning and sequencing is a hybridoma produced by obtaining a B cell from the transgenic mouse ami fusing the B cell to an immortal cell. An advantage of using hybridonias is that they can be easily screened, and a hybridoma that produces a human monoclonal antibody of interest selected. Alternatively, RNA can be isolated from B cells (or whole spleen) of the immunized animal. When sources other than hybridomas are used, it may be desirable io screen for sequences encoding immunoglobulins or immunoglobulin polypeptides with specific binding characteristics. One method for such screening is the use of phage display technology. Phage display is described in e.g., Dower et al., WO 91/1727 L McCafferty et al., WO 92/01047, and Caton and Koprowski, Proc. Natl. Acad. Sci. USA. 87:6450-6454 (1990), each of which is incorporated herein by reference. In one embodiment using phage display technology, cDNA from an immunized transgenic mouse (e.g., total spleen cDNA) is isolated, PCR is used to amplify cDNA sequences that encode a portion of an immunoglobulin polypeptide, e.g., CDR regions, and the amplified sequences are inserted into a phage vector, cDNAs encoding peptides of interest, e.g., variable region peptides with desired binding characteristics, are identified by standard techniques such as panning. The sequence of the amplified or cloned nucleic acid is then determined. Typically the sequence encoding an entire variable region of the immunoglobulin polypeptide is determined, however, sometimes only a portion of a variable region need be sequenced, for example, the CDR-encoding portion. Typically the sequenced portion will be at least 30 bases in length, and more often bases coding for at least about one-third or at least about one-half of the length of the variable region will be sequenced. Sequencing can be carried out on clones isolated from a cDNA library or, when PCR is used, after subcloning the amplified sequence or by direct PCR sequencing of the amplified segment. Sequencing is carried out using standard techniques (see, e.g., Sambrook et al. (1989) Molecular Cloning: A Laboratory Guide, Vols 1-3, Gold Spring Harbor Press, and Sanger, F. et al. (1977) Proc. Natl. Acad, Sci. USA 74:5463-5467, which is incorporated herein by reference). By comparing live sequence of the cloned nucleic acid with published sequences of human immunoglobulin genes and cDNAs, an artisan can determine readily, depending on the region sequenced, (i) the germline segment usage of the hybridoma immunoglobulin polypeptide (including the isotypc of the heavy chain) and (ii) the sequence of the heavy and light chain variable regions, including sequences resulting from N-region addition and the process of somatic mutation. One source of immunoglobulin gene sequence information is the National Center for Biotechnology Information, National Library of Medicine, National Institutes of Health, Betliesda, Md.

Once isolated, the DNA may beoperably linked to expression control sequences or placed into expression vectors, which are then transfected into host cells such as E. coli cells, simian COS cells. Chinese hamster ovary (CHO) cells, or myeloma cells that do not otherwise produce immunoglobulin protein, to direct the synthesis of monoclonal antibodies in the recombinant host cells.

Expression control sequences denote DNA sequences necessary for the expression of an operably linked coding sequence in a particular host organism., The control sequences that are suitable for prokaryotes, for example, include a promoter, optionally an operator sequence, and a ribosome-binding site. Eukaryotic cells are known to utilize promoters, polyadcnylalion signals, and enhancers.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome-binding site is operably linked to a coding sequence if it is positioned so as to facilitate translation. Generally, operably linked means thai the DNA sequences being linked are contiguous, and, in the case of a secretory leader, contiguous and in reading phase. However, enhancers do not have to be contiguous. Linking can be accomplished by ligation at convenient restriction sites. If such sites do not exist synthetic oligonucleotide adaptors or linkers can be used in accordance with conventional practice.

Cell, cell line, and cell culture are often used interchangeably and all such designations include progeny. Transformants and transformed cells include the primary subject cell and cultures derived therefrom without regard for the number of transfers. It also is understood that all progeny may not be precisely identical in DNA content, due to deliberate: or inadvertent mutations. Mutant progeny that have the same function or biological activity as screened for in the originally transformed cell are included.

Isolated nucleic acids also are provided that encode specific antibodies, optionally operably linked to control sequences recognized by a host cell, vectors and host cells comprising ihe nucleic acids, and recombinant techniques for the production of the antibodies, which may comprise culturing the host cell so that the nucleic acid is expressed and, optionally, recovering the antibody from the host cell culture or culture medium.

A variety of vectors are known in the art. Vector components can include one or moreof the following: a signal sequence (that, for example, can direct, secretion of the antibody), an origin of replication, one or more selective marker genes (that, for example, can confer antibiotic or other drug resistance, complement auxotrophic deficiencies, or supply critical nutrients not available in the media), an enhancer element, a promoter, and a transcription termination sequence, all of which are well known in the art.

Suitable host cells include prokaryote, yeast, or higher eukaryote cells. Suitable prokaryotes include eubacteria, such as Gram-negative or Gram-positive organisms, for example, Enterohactcriaecae such as Escherichia, e.g., E. coli, Enterobacter, Erwinia, Klebsiella, Proteus, Salmonella, e.g., Salmonella typhimurium, Serratia, e.g., Serratia marceseans, and Shigella, as well as Bacilli such as B. subtilis and B. licheniformis, Pseudomonas, and Streptomyces. In addition to prokaryotes, eukaryotic microbes such as filamentous fungi or yeast are suitable cloning or expression hosts for antibody-encoding vectors. Saccharbmyces cercvisiae, or common baker's yeast, is the most commonly used among lower eukaryotic host microorganisms. However, a number of other genera, species, and strains are commonly available, such as Pichia, e.g. P. pastoris, Schizosaccharomyces pombe; Kluyyeromyces, Yarrowia; Candida; Trichoderma reesia; Neurospora crassa; Schwanniomyces such as Schwanniomyces occidentalis; and filamentous fungi such as, e.g., Neurospora, Penicillium, Tolypocladium, and Aspergillus hosts such as A nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar). Aedes aegypti (mosquito). Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

However, interest has been greatest in vertebrate cells, and propagation of vertebrate cells in culture (tissue culture) has become routine. Examples of useful mammalian host cell-lines are Chinese hamster ovary cells, including CHOKI cells (ATCC CCL61) and Chinese hamster ovary ccll/-DHFR (DXB-11, DG-44; Urlaub et al. Proc. Natl. Acad. Sci. USA 77; 4216 ( 1980)): monkey kidney CV1 line transformed by SV40 (COS-7. ATCC CRL 1651): human embryonic kidney line (293 or 293 cells subcloned for growth in suspension culture, [Graham et al., J. Gen Virol. 36: 59 (1977)]: baby hamster kidney cells (BHK, ATCC CGL 10): mouse Sertoli cells (TM4, Mather, Biol. Reprod. 23: 243-251 (1980)); monkey kidney cells (CV1 ATCC CCL 70): African green,monkey kidney cells (VERO-76, ATCC CRL-1587): human cervical carcinoma cells (HELA. ATCC CCL 2); canine kidney cells (MDCK. ATCC CCL 34); buffalo rat liver cells (BRL 3A, ATCC CRL 1442); human lung cells (WI38, ATCC CCL 75); human hepatoma cells (Hep G2, HB 8065): mouse mammary tumor (MMT 060562, ATCC CCL51): TRI cells (Mather et al., Annals N.Y. Acad. Sci. 383: 44-68 (1982)); MRC 5 cells and FS4 cells.

The host cells can be cultured in a variety of media. Commercially available media such as Ham's F10 (Sigma). Minimal Essential Medium ((MEM), (Sigma)., RPMI-1640 (Sigma), and Dulbecco's Modified Eagle's Medium ((DMEM), Sigma) are suitable for culturing the host cells. In addition, any of the media described in Ham et al, Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 can be used as culture media for the host cells. Any of these media can be supplemented as necessary with hormones and/or oilier growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin TM. drug), trace elements (defined as inorganic compounds usually present at final concentrations in the micromolar range), and glucose or an equivalent energy source. Any other necessary supplements also can be included at appropriate concentrations that would be known to those skilled in the art. The culture conditions, such as temperature, pH, and the like, are those previously used with the host cell selected for expression, and will be apparent to the artisan.

The antibody composition can be purified using, for example, hydroxylapatite chromatography, cation or anion exchange chromatography, or preferably affinity chromatography, using the antigen of interest or protein A or protein G asan affinity ligand. Protein A can be used to purify antibodies thai are based on human gamma 1, gamma 2, or gamma 4heavy chains (Lindmark et al., J. Immunol. Meth. 62: 1-13 (1983)). Protein G is recommended for all mouse isotypes and for human gamma.3 (Guss et al., 20 EMBO J. 5: 15671575 (1986)). The mairix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX.TM. resin (J. T. Baker, Phillipsburg, 25 N.J.) is useful for purification. Other techniques for protein purification such as ethanol precipitation. Reverse Phase HPLC. chromatofocusing, SDS-PAGE, and ammonium sulfate precipitation are also possible depending on the specific binding agent or antibody to be recovered.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3-5, and more usually, at least 5 or 8-10 amino acids in a unique spatial conformation.

“Specifically binds” to or shows “specific binding” twoards an epitope means that the antibody reacts or associates more frequently, and/or more rapidly, and/or greater duration, and/or with greater affinity with the epitope than with, alternative substances.

In some embodimenls, once the subject's tumor has been analyzed to determine whether the tumor harbors a wild type PI3K-α versus a mutant PI3K-α, for example, PI3K-α E545K of PI3K-α H1047R, using any one or more of the assays and methods described above, a treatment, regimen can be prepared for the subject. If the subject's tumor harbors a PI3K-α having a mutation at position 1047, (for example, H1047R), the treatment regimen comprises administering to the subject a therapeutically effective amount of a PI3K-α selective inhibitor compound, or a dual PI3K-α/mTOR selective inhibitor, or a combination of a PI3K-α selective inhibitor or a mTOR selective inhibitor. If the subject's tumor harbors a PI3K-α having a mutation al position 545, (for example, E545K), the treatment regimen comprises administering to the subject a therapeutically effective amount of a combination of a PI3K-α selective inhibitor and a PI3K-β selective inhibitor, a dual PI3K-α/mTOR selective inhibitor, or a combination of a PI3K-α selective inhibitor and a mTOR selective inhibitor.

In another embodiment, the present invention provides kits comprising materials useful for carrying out the methods of the invention. The diagnostic/screening procedures described herein may be performed by diagnostic laboratories, experimental laboratories, or practitioners. The invention provides kits which can be used in these different settings.

Basic materials and reagents required for identifying a Pl3K-α mutation in a subject's tumor or cancer according to methods of the present invention may be assembled together in a kit. In certain embodiments, the kit comprises at least one PI3K-α amino acid sequence determining reagent that specifically detects a mutation in a nucleic acid or protein obtained from a subject's tumor disclosed herein, and instructions for using the kit according to one or more methods of the invention. Each kit necessarily comprises reagents which render the procedure specific. Thus, for delecting mRNA harboring the PI3K-α H047R or E545K mutation, the reagent will comprise a nucleic acid probe complementary to mRNA, such as, for example, a cDNA or an oligonucleotide. The nucleic acid probe may or may not be immobilized on a substrate surface (e.g., a microarray). For detecting a polypeptide product encoded by at least one PI3K-α mutation gene, the reagent will comprise an antibody that specifically binds to the mutated PI3K-α or a wild-type PI3K-α.

Depending on the procedure, the kit may further comprise one or more of: extraction buffer and/or reagents, amplification buffer and/or reagents, hybridization buffer and/or reagents, immunodetection buffer and/or reagents, labeling buffer and/or reagents, and detection means. Protocols for using these buffers and reagents for performing different steps of the procedure may also be included in the kit.

Reagents may be supplied in a solid (e.g.. lyophilized) or liquid form. Kits of the present invention may optionally comprise one or more receptacles for mixing samples and/or reagents (e.g., vial, ampoule, test tube, ELISA plate, culture plate, flask or bottle) for each individual buffer and/or reagent. Each component will generally be suitable as aliquoted in its respective container or provided in a concentrated form. Other containers suitable for conducting certain steps for the disclosed methods may also be provided. The individual containers of the kit are preferably maintained in close confinement for commercial sale.

In certain embodiments, the kits of the present invention further comprise control samples. For example, a kit may include samples of total mRNA derived from tissue of various physiological states, such as, for example, wild-type PI3K-α, PI3K-α H1047R mRNA or PI3K-α E545K mRNA to be used as controls. In other embodiments, the inventive kits comprise at least one prostate disease expression profile map as described herein for use as comparison template. Preferably, the expression profile map is digital information stored in a computer-readable medium;

Instructions for using the kit according to one or more methods of the invention may comprise instructions for processing the prostate tissue sample and/or performing the test, instructions for interpreting the results as well as a notice in the form prescribed by a governmental agency (e.g., EDA) regulating the manufacture, use or sale of pharmaceuticals or biological products.

### Representative Compounds

Representative compounds of Formula I are depicted in the following tables. The examples are merely illustrative and do not limit the scope of the invention in any way. Compounds of the invenlion are named according to systematic application of the nomenclature rules agreed upon by the International Union of Pure and Applied Chemistry (IUPAC). International Union of Biochemistry and Molecular Biology (IUBMB), and the Chemical Abstracts Service (CAS). Specifically, the names in the tables below were generated using ACD/Labs naming software 8.00 release, product version 8.08 or later.

In one embodiment, compounds of the invention are listed below.

In one embodiment, compounds of the invenlion are listed in Table I

TABLE 1

| CMPD | STRUCTURE | NAME |
|---|---|---|
| 2 | 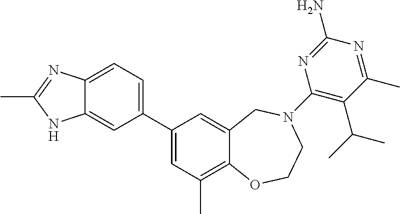 | 4-methyl-5-(1-methylethyl)-6-[9- methyl-7-(2-methyl-1H-benzimidazol- 6-yl)-2,3-dihydro-1,4-benzoxazepin- 4(5H)-yl]pyrimidin-2-amine |
| 3 | 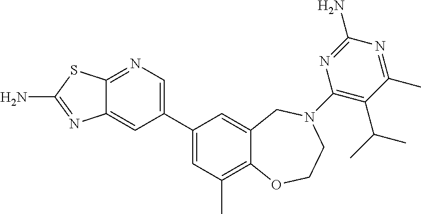 [SMILES: Cc1cc(-c2cnc3sc(N)nc3c2)cc2c1OCCN(c1nc(N)nc(C)c1C(C)C)C2] | 6-{4-[2-amino-6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 5 | 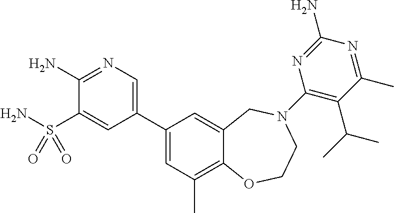 [SMILES: Cc1cc(-c2cnc(N)c(S(N)(=O)=O)c2)cc2c1OCCN(c1nc(N)nc(C)c1C(C)C)C2] | 2-amino-5-{4-[2-amino-6-methyl-5- (1-methylethyl)pyrimidin-4-yl]-9- methyl-2,3,4,5-tetrahydro-1,4- benzoxazepin-7-yl}pyridine-3- sulfonamide |
| 6 | 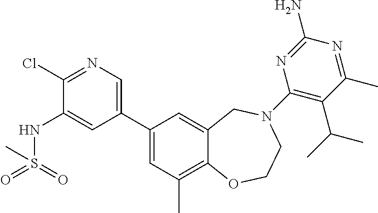 [SMILES: Cc1cc(-c2cnc(Cl)c(NS(C)(=O)=O)c2)cc2c1OCCN(c1nc(N)nc(C)c1C(C)C)C2] | N-(5-{4-[2-amino-6-methyl-5-(1- methylethyl)pyrimidin-4-yl]-9- methyl-2,3,4,5-tetrahydro-1,4- benzoxazepin-7-yl}-2-chloropyridin- 3-yl)methanesulfonamide |
| 7 | 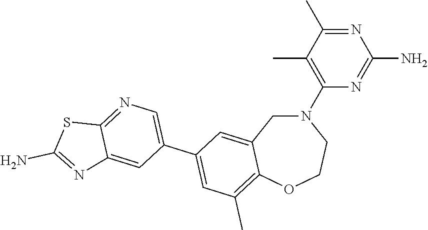 [SMILES: Cc1cc(-c2cnc3sc(N)nc3c2)cc2c1OCCN(c1nc(N)nc(C)c1C)C2] | 6-[4-(2-amino-5,6-dimethylpyrimidin- 4-yl)-9-methyl-2,3,4,5-tetrahydro-1,4- benzoxazepin-7-yl][1,3]thiazolo[5,4- b]pyridin-2-amine |
| 8 | 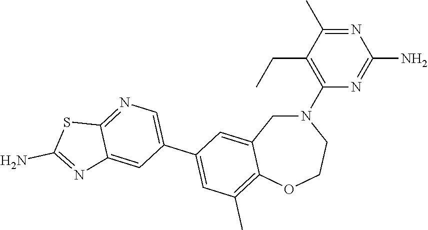 [SMILES: CCc1c(C)nc(N)nc1N1CCOc2c(C)cc(-c3cnc4sc(N)nc4c3)cc2C1] | 6-[4-(2-amino-5-ethyl-6- methylpyrimidin-4-yl)-9-methyl- 2,3,4,5-tetrahydro-1,4-benzoxazepin- 7-yl][1,3]thiazolo[5,4-b]pyridin-2- amine |
| 9 | 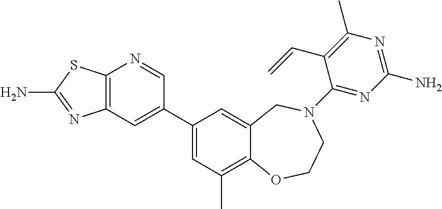 [SMILES: C=Cc1c(C)nc(N)nc1N1CCOc2c(C)cc(-c3cnc4sc(N)nc4c3)cc2C1] | 6-[4-(2-amino-5-ethenyl-6- methylpyrimidin-4-yl)-9-methyl- 2,3,4,5-tetrahydro-1,4-benzoxazepin- 7-yl][1,3]thiazolo[5,4-b]pyridin-2- amine |
| 10 | 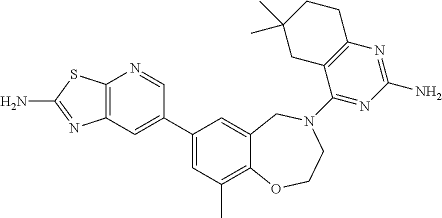 [SMILES: Cc1cc(-c2cnc3sc(N)nc3c2)cc2c1OCCN(c1nc(N)nc3c1CC(C)(C)CC3)C2] | 4-[7-(2-amino[1,3]thiazolo[5,4- b]pyridin-6-yl)-9-methyl-2,3-dihydro- 1,4-benzoxazepin-4(5H)-yl]-6,6- dimethyl-5,6,7,8- tetrahydroquinazolin-2-amine |
| 11 | 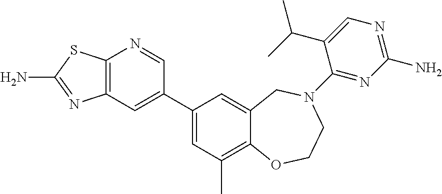 [SMILES: Cc1cc(-c2cnc3sc(N)nc3c2)cc2c1OCCN(c1nc(N)ncc1C(C)C)C2] | 6-{4-[2-amino-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 12 | 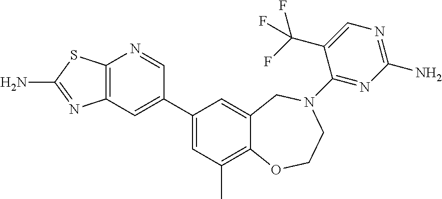 [SMILES: Cc1cc(-c2cnc3sc(N)nc3c2)cc2c1OCCN(c1nc(N)ncc1C(F)(F)F)C2] | 6-{4-[2-amino-5- (trifluoromethyl)pyrimidin-4-yl]-9- methyl-2,3,4,5-tetrahydro-1,4- benzoxazepin-7-yl}[1,3]thiazolo[5,4- b]pyridin-2-amine |
| 13 | 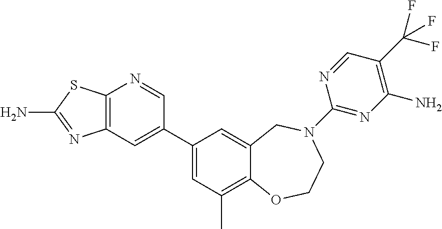 [SMILES: Cc1cc(-c2cnc3sc(N)nc3c2)cc2c1OCCN(c1ncc(C(F)(F)F)c(N)n1)C2] | 6-{4-[4-amino-5- (trifluoromethyl)pyrimidin-2-yl]-9- methyl-2,3,4,5-tetrahydro-1,4- benzoxazepin-7-yl}[1,3]thiazolo[5,4- b]pyridin-2-amine |
| 14 | 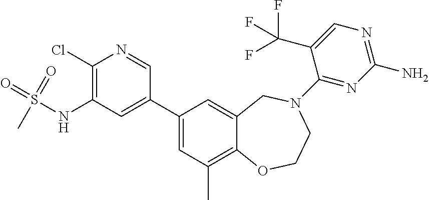 [SMILES: Cc1cc(-c2cnc(Cl)c(NS(C)(=O)=O)c2)cc2c1OCCN(c1nc(N)ncc1C(F)(F)F)C2] | N-(5-{4-[2-amino-5- (trifluoromethyl)pyrimidin-4-yl]-9- methyl-2,3,4,5-tetrahydro-1,4- benzoxazepin-7-yl}-2-chloropyridin- 3-yl)methanesulfonamide |
| 15 | 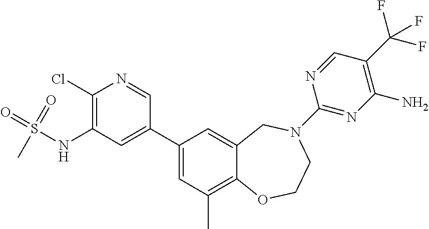 [SMILES: Cc1cc(-c2cnc(Cl)c(NS(C)(=O)=O)c2)cc2c1OCCN(c1ncc(C(F)(F)F)c(N)n1)C2] | N-(5-{4-[4-amino-5- (trifluoromethyl)pyrimidin-2-yl]-9- methyl-2,3,4,5-tetrahydro-1,4- benzoxazepin-7-yl}-2-chloropyridin- 3-yl)methanesulfonamide |
| 16 | 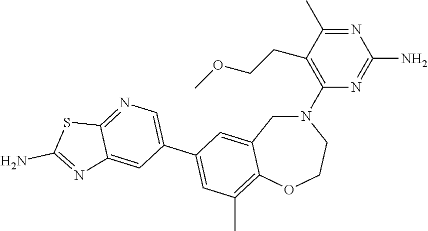 [SMILES: COCCc1c(C)nc(N)nc1N1CCOc2c(C)cc(-c3cnc4sc(N)nc4c3)cc2C1] | 6-(4-{2-amino-6-methyl-5-[2- (methyloxy)ethyl]pyrimidin-4-yl}-9- methyl-2,3,4,5-tetrahydro-1,4- benzoxazepin-7-yl)[1,3]thiazolo[5,4- b]pyridin-2-amine |
| 17 | 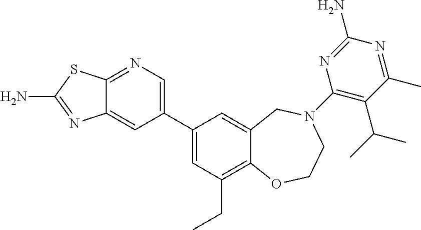 [SMILES: CCc1cc(-c2cnc3sc(N)nc3c2)cc2c1OCCN(c1nc(N)nc(C)c1C(C)C)C2] | 6-{4-[2-amino-6-methyl-5-(1- methylethyl)pyrimidin-4-yl]-9-ethyl- 2,3,4,5-tetrahydro-1,4-benzoxazepin- 7-yl}[1,3]thiazolo[5,4-b]pyridin-2- amine |
| 18 | 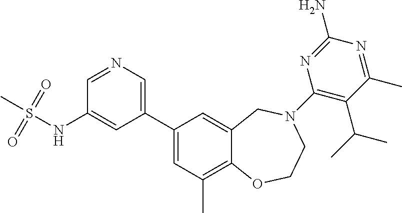 | N-(5-{4-[2-amino-6-methyl-5-(1- methylethyl)pyrimidin-4-yl]-9- methyl-2,3,4,5-tetrahydro-1,4- benzoxazepin-7-yl}pyridin-3- yl)methanesulfonamide |
| 19 | 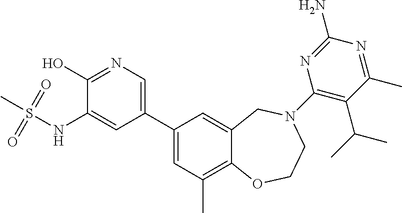 | N-(5-{4-[2-amino-6-methyl-5-(1- methylethyl)pyrimidin-4-yl]-9- methyl-2,3,4,5-tetrahydro-1,4- benzoxazepin-7-yl}-2- hydroxypyridin-3- yl)methanesulfonamide |
| 20 | 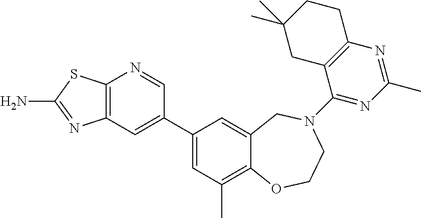 [SMILES: Cc1nc2c(c(N3CCOc4c(C)cc(-c5cnc6sc(N)nc6c5)cc4C3)n1)CC(C)(C)CC2] | 6-[9-methyl-4-(2,6,6-trimethyl- 5,6,7,8-tetrahydroquinazolin-4-yl)- 2,3,4,5-tetrahydro-1,4-benzoxazepin- 7-yl][1,3]thiazolo[5,4-b]pyridin-2- amine |
| 21 | 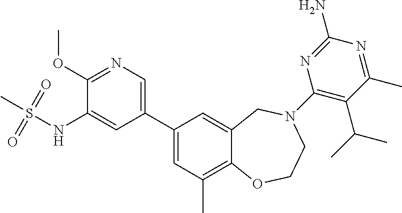 | N-[5-{4-[2-amino-6-methyl-5-(1- methylethyl)pyrimidin-4-yl]-9- methyl-2,3,4,5-tetrahydro-1,4- benzoxazepin-7-yl}-2- (methyloxy)pyridin-3- yl]methanesulfonamide |
| 22 | 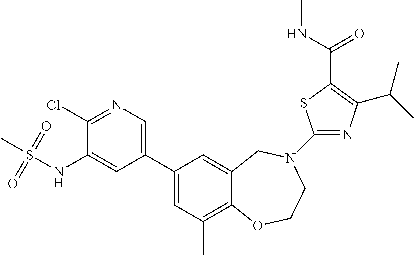 [SMILES: CNC(=O)c1sc(N2CCOc3c(C)cc(-c4cnc(Cl)c(NS(C)(=O)=O)c4)cc3C2)nc1C(C)C] | 2-[7-{6-chloro-5- [(methylsulfonyl)amino]pyridin-3-yl}- 9-methyl-2,3-dihydro-1,4- benzoxazepin-4(5H)-yl]-N-methyl-4- (1-methylethyl)-1,3-thiazole-5- carboxamide |
| 23 | 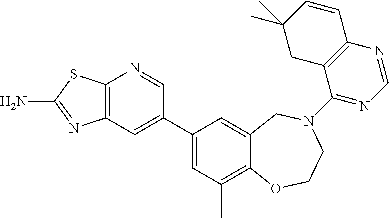 [SMILES: Cc1cc(-c2cnc3sc(N)nc3c2)cc2c1OCCN(c1ncnc3c1CC(C)(C)C=C3)C2] | 6-[4-(6,6-dimethyl-5,6- dihydroquinoazolin-4-yl)-9-methyl- 2,3,4,5-tetrahydro-1,4-benzoxazepin- 7-yl][1,3]thiazolo[5,4-b]pyridin-2- amine |
| 24 | 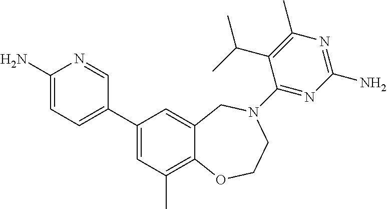 [SMILES: Cc1cc(-c2ccc(N)nc2)cc2c1OCCN(c1nc(N)nc(C)c1C(C)C)C2] | 4-[7-(6-aminopyridin-3-yl)-9-methyl- 2,3-dihydro-1,4-benzoxazepin-4(5H)- yl]-6-methyl-5-(1- methylethyl)pyrimidin-2-amine |
| 25 | 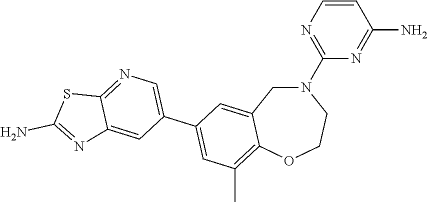 [SMILES: Cc1cc(-c2cnc3sc(N)nc3c2)cc2c1OCCN(c1nccc(N)n1)C2] | 6-[4-(4-aminopyrimidin-2-yl)-9- methyl-2,3,4,5-tetrahydro-1,4- benzoxazepin-7-yl][1,3]thiazolo[5,4- b]pyridin-2-amine |
| 26 | 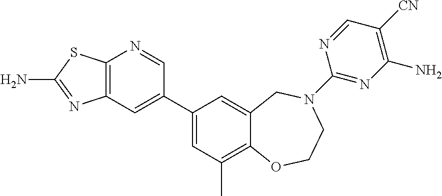 [SMILES: Cc1cc(-c2cnc3sc(N)nc3c2)cc2c1OCCN(c1ncc(C#N)c(N)n1)C2] | 4-amino-2-[7-(2- amino[1,3]thiazolo[5,4-b]pyridin-6- yl)-9-methyl-2,3-dihydro-1,4- benzoxazepin-4(5H)-yl]pyrimidine-5- carbonitrile |
| 27 | 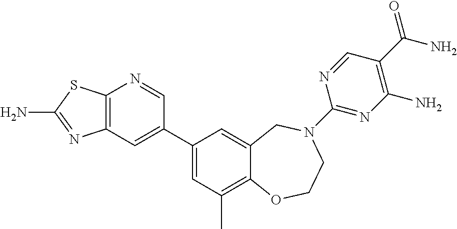 [SMILES: Cc1cc(-c2cnc3sc(N)nc3c2)cc2c1OCCN(c1ncc(C(N)=O)c(N)n1)C2] | 4-amino-2-[7-(2- amino[1,3]thiazolo[5,4-b]pyridin-6- yl)-9-methyl-2,3-dihydro-1,4- benzoxazepin-4(5H)-yl]pyrimidine-5- carboxamide |
| 28 | 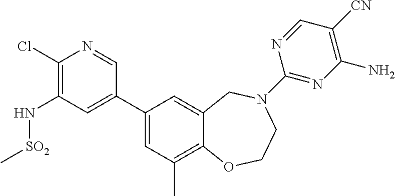 [SMILES: Cc1cc(-c2cnc(Cl)c(NS(C)(=O)=O)c2)cc2c1OCCN(c1ncc(C#N)c(N)n1)C2] | N-{5-[4-(4-amino-5-cyanopyrimidin- 2-yl)-9-methyl-2,3,4,5-tetrahydro-1,4- benzoxazepin-7-yl]-2-chloropyridin-3- yl}methanesulfonamide |
| 30 | 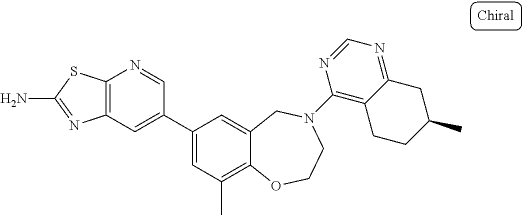 [SMILES: Cc1cc(-c2cnc3sc(N)nc3c2)cc2c1OCCN(c1ncnc3c1CC[C@H](C)C3)C2] | 6-{9-methyl-4-[(7S)-7-methyl-5,6,7,8- tetrahydroquinazolin-4-yl]-2,3,4,5- tetrahydro-1,4-benzoxazepin-7- yl}[1,3]thiazolo[5,4-b]pyridin-2- amine |
| 31 | 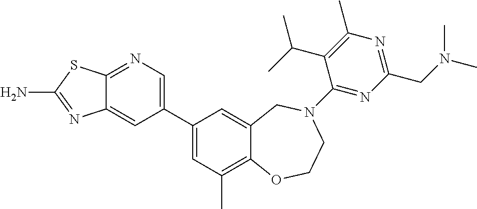 [SMILES: Cc1cc(-c2cnc3sc(N)nc3c2)cc2c1OCCN(c1nc(CN(C)C)nc(C)c1C(C)C)C2] | 6-(4-{2-[(dimethylamino)methyl]-6- methyl-5-(1-methylethyl)pyrimidin-4- yl}-9-methyl-2,3,4,5-tetrahydro-1,4- benzoxazepin-7-yl)[1,3]thiazolo[5,4- b]pyridin-2-amine |
| 32 | 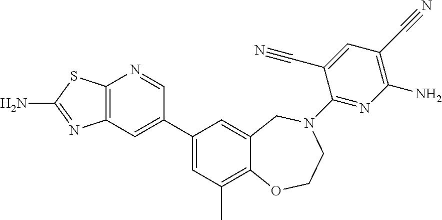 [SMILES: Cc1cc(-c2cnc3sc(N)nc3c2)cc2c1OCCN(c1nc(N)c(C#N)cc1C#N)C2] | 2-amino-6-[7-(2- amino[1,3]thiazolo[5,4-b]pyridin-6- yl)-9-methyl-2,3-dihydro-1,4- benzoxazepin-4(5H)-yl]pyridine-3,5- dicarbonitrile |
| 33 | 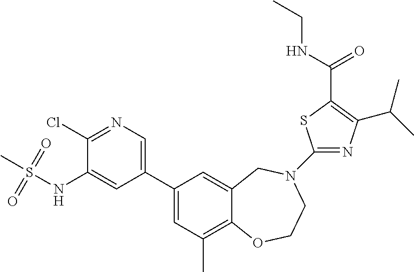 [SMILES: CCNC(=O)c1sc(N2CCOc3c(C)cc(-c4cnc(Cl)c(NS(C)(=O)=O)c4)cc3C2)nc1C(C)C] | 2-[7-{6-chloro-5- [(methylsulfonyl)amino]pyridin-3-yl}- 9-methyl-2,3-dihydro-1,4- benzoxazepin-4(5H)-yl]-N-ethyl-4-(1- methylethyl)-1,3-thiazole-5- carboxamide |
| 34 | 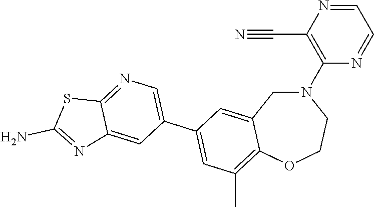 [SMILES: Cc1cc(-c2cnc3sc(N)nc3c2)cc2c1OCCN(c1nccnc1C#N)C2] | 3-[7-(2-amino[1,3]thiazolo[5,4- b]pyridin-6-yl)-9-methyl-2,3-dihydro- 1,4-benzoxazepin-4(5H)-yl]pyrazine- 2-carbonitrile |
| 35 | 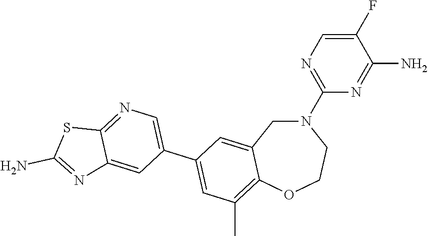 [SMILES: Cc1cc(-c2cnc3sc(N)nc3c2)cc2c1OCCN(c1ncc(F)c(N)n1)C2] | 6-[4-(4-amino-5-fluoropyrimidin-2- yl)-9-methyl-2,3,4,5-tetrahydro-1,4- benzoxazepin-7-yl][1,3]thiazolo[5,4- b]pyridin-2-amine |
| 36 | 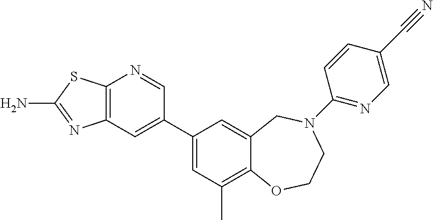 [SMILES: Cc1cc(-c2cnc3sc(N)nc3c2)cc2c1OCCN(c1ccc(C#N)cn1)C2] | 6-[7-(2-amino[1,3]thiazolo[5,4- b]pyridin-6-yl)-9-methyl-2,3-dihydro- 1,4-benzoxazepin-4(5H)-yl]pyridine- 3-carbonitrile |
| 37 | 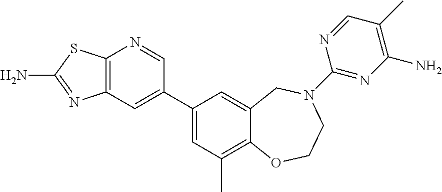 [SMILES: Cc1cnc(N2CCOc3c(C)cc(-c4cnc5sc(N)nc5c4)cc3C2)nc1N] | 6-[4-(4-amino-5-methylpyrimidin-2- yl)-9-methyl-2,3,4,5-tetrahydro-1,4- benzoxazepin-7-yl][1,3]thiazolo[5,4- b]pyridin-2-amine |
| 38 | 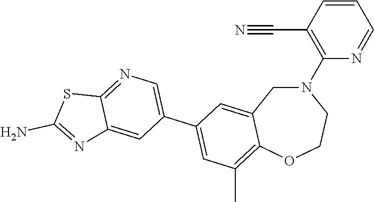 [SMILES: Cc1cc(-c2cnc3sc(N)nc3c2)cc2c1OCCN(c1ncccc1C#N)C2] | 2-[7-(2-amino[1,3]thiazolo[5,4- b]pyridin-6-yl)-9-methyl-2,3-dihydro- 1,4-benzoxazepin-4(5H)-yl]pyridine- 3-carbonitrile |
| 39 | 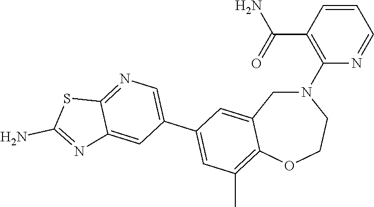 [SMILES: Cc1cc(-c2cnc3sc(N)nc3c2)cc2c1OCCN(c1ncccc1C(N)=O)C2] | 2-[7-(2-amino[1,3]thiazolo[5,4- b]pyridin-6-yl)-9-methyl-2,3-dihydro- 1,4-benzoxazepin-4(5H)-yl]pyridine- 3-carboxamide |
| 40 | 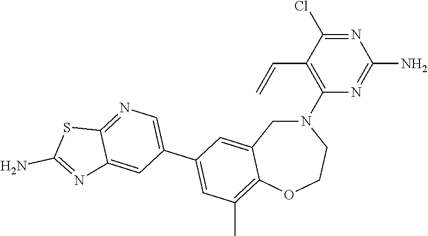 [SMILES: C=Cc1c(Cl)nc(N)nc1N1CCOc2c(C)cc(-c3cnc4sc(N)nc4c3)cc2C1] | 6-[4-(2-amino-6-chloro-5- ethenylpyrimidin-4-yl)-9-methyl- 2,3,4,5-tetrahydro-1,4-benzoxazepin- 7-yl][1,3]thiazolo[5,4-b]pyridin-2- amine |
| 41 | 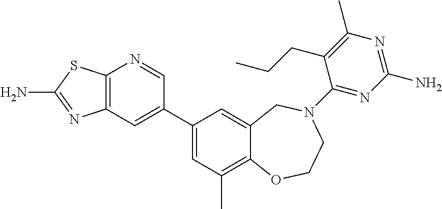 [SMILES: CCCc1c(C)nc(N)nc1N1CCOc2c(C)cc(-c3cnc4sc(N)nc4c3)cc2C1] | 6-[4-(2-amino-6-methyl-5- propylpyrimidin-4-yl)-9-methyl- 2,3,4,5-tetrahydro-1,4-benzoxazepin- 7-yl][1,3]thiazolo[5,4-b]pyridin-2- amine |
| 42 | 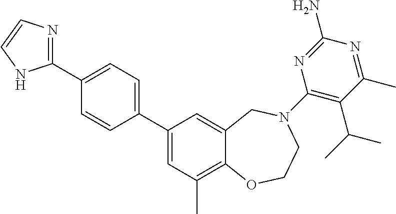 [SMILES: Cc1cc(-c2ccc(-c3ncc[nH]3)cc2)cc2c1OCCN(c1nc(N)nc(C)c1C(C)C)C2] | 4-{7-[4-(1H-imidazol-2-yl)phenyl]-9- methyl-2,3-dihydro-1,4-benzoxazepin- 4(5H)-yl}-6-methyl-5-(1- methylethyl)pyrimidin-2-amine |
| 43 | 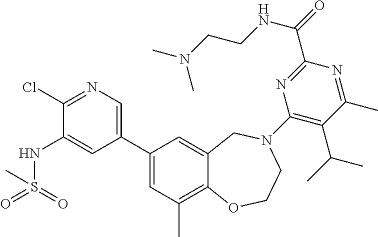 [SMILES: Cc1cc(-c2cnc(Cl)c(NS(C)(=O)=O)c2)cc2c1OCCN(c1nc(C(=O)NCCN(C)C)nc(C)c1C(C)C)C2] | 4-[7-(6-chloro-5- [(methylsulfonyl)amino]pyridin-3-yl}- 9-methyl-2,3-dihydro-1,4- benzoxazepin-4(5H)-yl]-N-[2- (dimethylamino)ethyl]-6-methyl-5-(1- methylethyl)pyrimidine-2- carboxamide |
| 44 | 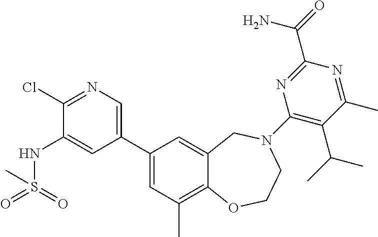 [SMILES: Cc1cc(-c2cnc(Cl)c(NS(C)(=O)=O)c2)cc2c1OCCN(c1nc(C(N)=O)nc(C)c1C(C)C)C2] | 4-[7-{6-chloro-5- [(methylsulfonyl)amino]pyridin-3-yl}- 9-methyl-2,3-dihydro-1,4- benzoxazepin-4(5H)-yl]-6-methyl-5- (1-methylethyl)pyrimidine-2- carboxamide |
| 45 | 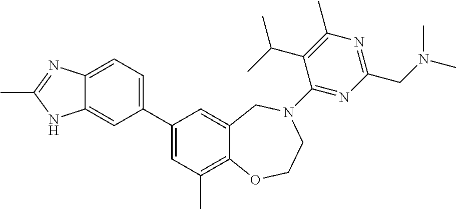 | N,N-dimethyl-1-{4-methyl-5-(1- methylethyl)-6-[9-methyl-7-(2- methyl-1H-benzimidazol-6-yl)-2,3- dihydro-1,4-benzoxazepin-4(5H)- yl]pyrimidin-2-yl}methanamine |
| 46 | 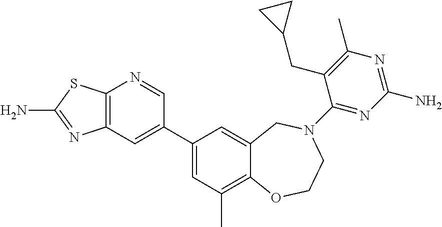 [SMILES: Cc1cc(-c2cnc3sc(N)nc3c2)cc2c1OCCN(c1nc(N)nc(C)c1CC1CC1)C2] | 6-{4-[2-amino-5-(cyclopropylmethyl)- 6-methylpyrimidin-4-yl]-9-methyl- 2,3,4,5-tetrahydro-1,4-benzoxazepin- 7-yl}[1,3]thiazolo[5,4-b]pyridin-2- amine |
| 47 | 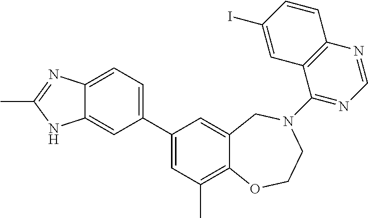 [SMILES: Cc1nc2ccc(-c3cc(C)c4c(c3)CN(c3ncnc5ccc(I)cc35)CCO4)cc2[nH]1] | 4-(6-iodoquinazolin-4-yl)-9-methyl-7- (2-methyl-1H-benzimidazol-6-yl)- 2,3,4,5-tetrahydro-1,4-benzoxazepine |
| 48 | 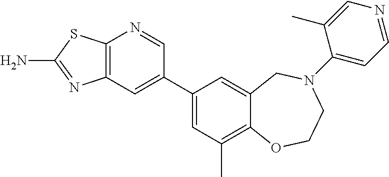 [SMILES: Cc1cnccc1N1CCOc2c(C)cc(-c3cnc4sc(N)nc4c3)cc2C1] | 6-[9-methyl-4-(3-methylpyridin-4-yl)- 2,3,4,5-tetrahydro-1,4-benzoxazepin- 7-yl][1,3]thiazolo[5,4-b]pyridin-2- amine |
| 49 | 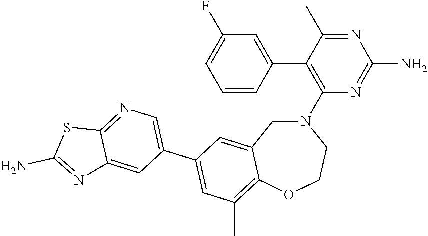 [SMILES: Cc1cc(-c2cnc3sc(N)nc3c2)cc2c1OCCN(c1nc(N)nc(C)c1-c1cccc(F)c1)C2] | 6-{4-[2-amino-5-(3-fluorophenyl)-6- methylpyrimidin-4-yl]-9-methyl- 2,3,4,5-tetrahydro-1,4-benzoxazepin- 7-yl}[1,3]thiazolo[5,4-b]pyridin-2- amine |
| 50 | 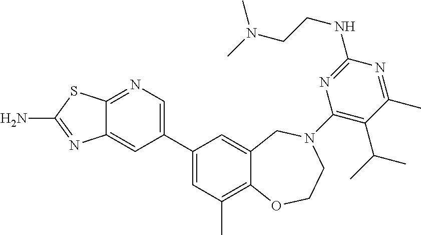 [SMILES: Cc1cc(-c2cnc3sc(N)nc3c2)cc2c1OCCN(c1nc(NCCN(C)C)nc(C)c1C(C)C)C2] | N′-{4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2-yl}- N,N-dimethylethane-1,2-diamine |
| 51 | 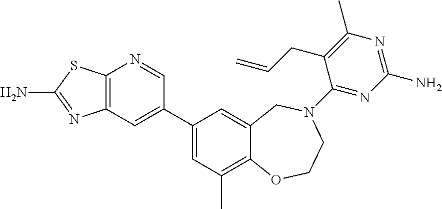 [SMILES: C=CCc1c(C)nc(N)nc1N1CCOc2c(C)cc(-c3cnc4sc(N)nc4c3)cc2C1] | 6-{4-(2-amino-6-methyl-5-prop-2-en- 1-ylpyrimidin-4-yl)-9-methyl-2,3,4,5- tetrahydro-1,4-benzoxazepin-7- yl][1,3]thiazolo[5,4-b]pyridin-2-amine |
| 52 | 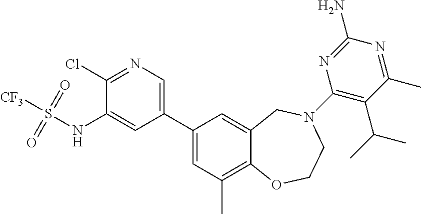 [SMILES: Cc1cc(-c2cnc(Cl)c(NS(=O)(=O)C(F)(F)F)c2)cc2c1OCCN(c1nc(N)nc(C)c1C(C)C)C2] | N-(5-{4-[2-amino-6-methyl-5-(1- methylethyl)pyrimidin-4-yl]-9- methyl-2,3,4,5-tetrahydro-1,4- benzoxazepin-7-yl}-2-chloropyridin- 3-yl)-1,1,1- trifluoromethanesulfonamide |
| 53 | 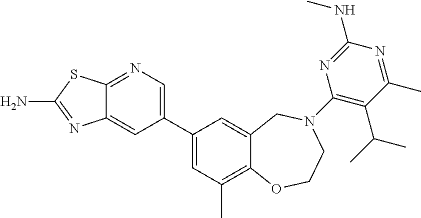 [SMILES: CNc1nc(C)c(C(C)C)c(N2CCOc3c(C)cc(-c4cnc5sc(N)nc5c4)cc3C2)n1] | 6-{9-methyl-4-[6-methyl-2- (methylamino)-5-(1- methylethyl)pyrimidin-4-yl]-2,3,4,5- tetrahydro-1,4-benzoxazepin-7- yl}[1,3]thiazolo[5,4-b]pyridin-2- amine |
| 54 | 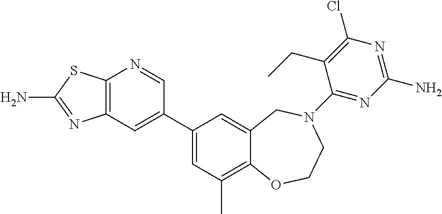 [SMILES: CCc1c(Cl)nc(N)nc1N1CCOc2c(C)cc(-c3cnc4sc(N)nc4c3)cc2C1] | 6-[4-(2-amino-6-chloro-5- ethylpyrimidin-4-yl)-9-methyl-2,3,4,5- tetrahydro-1,4-benzoxazepin-7- yl}[1,3]thiazolo[5,4-b]pyridin-2-amine |
| 55 | 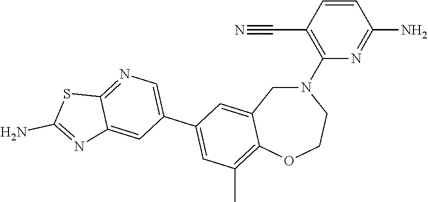 [SMILES: Cc1cc(-c2cnc3sc(N)nc3c2)cc2c1OCCN(c1nc(N)ccc1C#N)C2] | 6-amino-2-[7-(2- amino[1,3]thiazolo[5,4-b]pyridin-6- yl)-9-methyl-2,3-dihydro-1,4- benzoxazepin-4(5H)-yl]pyridine-3- carbonitrile |
| 56 | 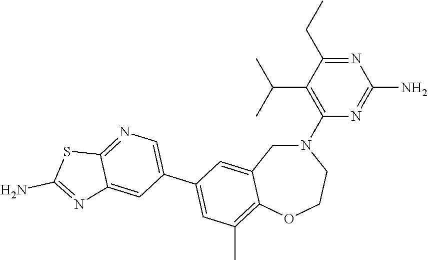 [SMILES: CCc1nc(N)nc(N2CCOc3c(C)cc(-c4cnc5sc(N)nc5c4)cc3C2)c1C(C)C] | 6-{4-[2-amino-6-ethyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 57 | 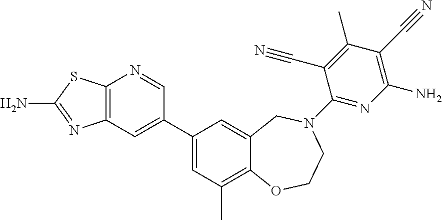 [SMILES: Cc1cc(-c2cnc3sc(N)nc3c2)cc2c1OCCN(c1nc(N)c(C#N)c(C)c1C#N)C2] | 2-amino-6-[7-(2- amino[1,3]thiazolo[5,4-b]pyridin-6- yl)-9-methyl-2,3-dihydro-1,4- benzoxazepin-4(5H)-yl]-4- methylpyridine-3,5-dicarbonitrile |
| 58 | 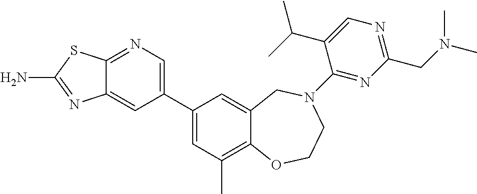 [SMILES: Cc1cc(-c2cnc3sc(N)nc3c2)cc2c1OCCN(c1nc(CN(C)C)ncc1C(C)C)C2] | 6-(4-{2-[(dimethylamino)methyl]-5- (1-methylethyl)pyrimidin-4-yl}-9- methyl-2,3,4,5-tetrahydro-1,4- benzoxazepin-7-yl)[1,3]thiazolo[5,4- b]pyridin-2-amine |
| 59 | 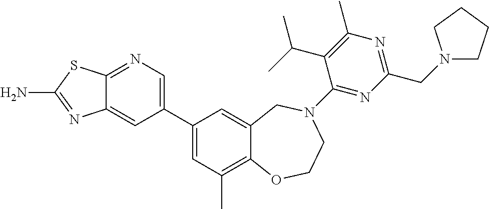 [SMILES: Cc1cc(-c2cnc3sc(N)nc3c2)cc2c1OCCN(c1nc(CN3CCCC3)nc(C)c1C(C)C)C2] | 6-{9-methyl-4-[6-methyl-5-(1- methylethyl)-2-(pyrrolidin-1- ylmethyl)pyrimidin-4-yl]-2,3,4,5- tetrahydro-1,4-benzoxazepin-7- yl}[1,3]thiazolo[5,4-b]pyridin-2- amine |
| 60 | 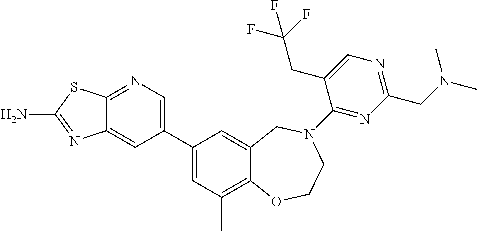 [SMILES: Cc1cc(-c2cnc3sc(N)nc3c2)cc2c1OCCN(c1nc(CN(C)C)ncc1CC(F)(F)F)C2] | 6-(4-{2-[(dimethylamino)methyl]-5- (2,2,2-trifluoroethyl)pyrimidin-4-yl}- 9-methyl-2,3,4,5-tetrahydro-1,4- benzoxazepin-7-yl)[1,3]thiazolo[5,4- b]pyridin-2-amine |
| 61 | 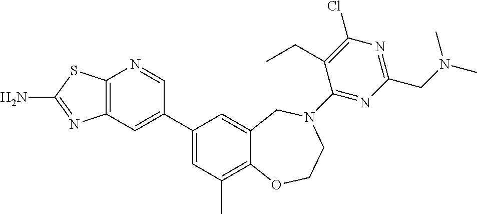 [SMILES: CCc1c(Cl)nc(CN(C)C)nc1N1CCOc2c(C)cc(-c3cnc4sc(N)nc4c3)cc2C1] | 6-(4-{6-chloro-2- [(dimethylamino)methyl]-5- ethylpyrimidin-4-yl}-9-methyl- 2,3,4,5-tetrahydro-1,4-benzoxazepin- 7-yl)[1,3]thiazolo[5,4-b]pyridin-2- amine |
| 62 | 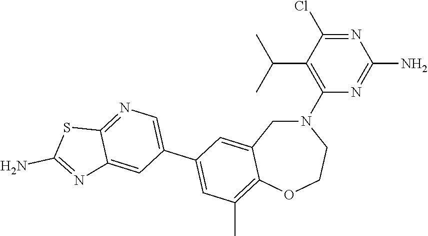 [SMILES: Cc1cc(-c2cnc3sc(N)nc3c2)cc2c1OCCN(c1nc(N)nc(Cl)c1C(C)C)C2] | 6-{4-[2-amino-6-chloro-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 63 | 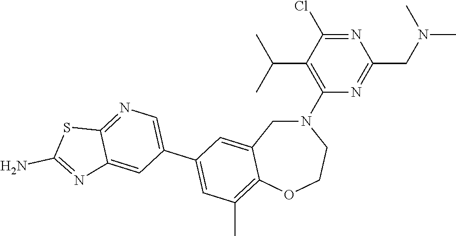 [SMILES: Cc1cc(-c2cnc3sc(N)nc3c2)cc2c1OCCN(c1nc(CN(C)C)nc(Cl)c1C(C)C)C2] | 6-(4-{6-chloro-2- [(dimethylamino)methyl]-5-(1- methylethyl)pyrimidin-4-yl}-9- methyl-2,3,4,5-tetrahydro-1,4- benzoxazepin-7-yl)[1,3]thiazolo[5,4- b]pyridin-2-amine |
| 64 | 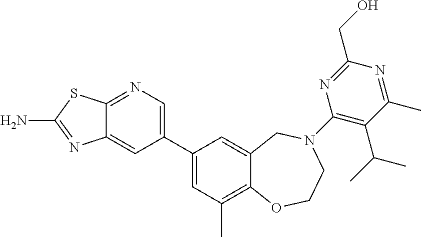 [SMILES: Cc1cc(-c2cnc3sc(N)nc3c2)cc2c1OCCN(c1nc(CO)nc(C)c1C(C)C)C2] | {4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2- yl}methanol |
| 65 | 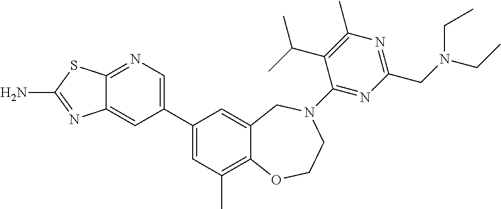 [SMILES: CCN(CC)Cc1nc(C)c(C(C)C)c(N2CCOc3c(C)cc(-c4cnc5sc(N)nc5c4)cc3C2)n1] | 6-(4-{2-[(diethylamino)methyl]-6- methyl-5-(1-methylethyl)pyrimidin-4- yl}-9-methyl-2,3,4,5-tetrahydro-1,4- benzoxazepin-7-yl)[1,3]thiazolo[5,4- b]pyridin-2-amine |
| 66 | 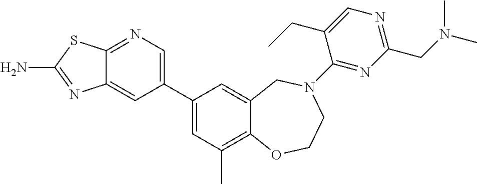 [SMILES: CCc1cnc(CN(C)C)nc1N1CCOc2c(C)cc(-c3cnc4sc(N)nc4c3)cc2C1] | 6-(4-{2-[(dimethylamino)methyl]-5- ethylpyrimidin-4-yl}-9-methyl- 2,3,4,5-tetrahydro-1,4-benzoxazepin- 7-yl)[1,3]thiazolo[5,4-b]pyridin-2- amine |
| 67 | 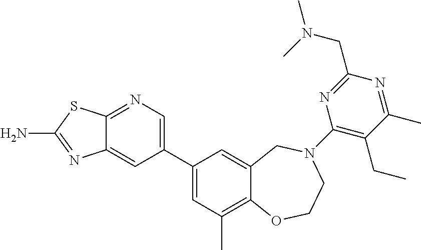 [SMILES: CCc1c(C)nc(CN(C)C)nc1N1CCOc2c(C)cc(-c3cnc4sc(N)nc4c3)cc2C1] | 6-(4-{2-[(dimethylamino)methyl]-5- ethyl-6-methylpyrimidin-4-yl}-9- methyl-2,3,4,5-tetrahydro-1,4- benzoxazepin-7-yl)[1,3]thiazolo[5,4- b]pyridin-2-amine |
| 68 | 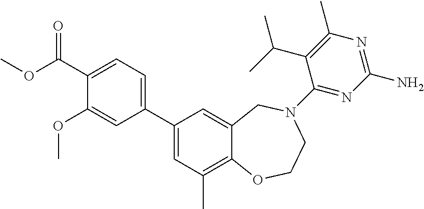 [SMILES: COC(=O)c1ccc(-c2cc(C)c3c(c2)CN(c2nc(N)nc(C)c2C(C)C)CCO3)cc1OC] | methyl 4-{4-[2-amino-6-methyl-5-(1- methylethyl)pyrimidin-4-yl]-9- methyl-2,3,4,5-tetrahydro-1,4- benzoxazepin-7-yl}-2- (methyloxy)benzoate |
| 69 | 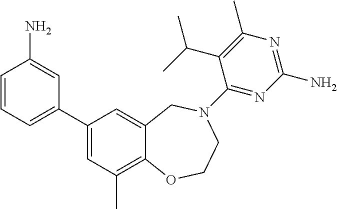 [SMILES: Cc1cc(-c2cccc(N)c2)cc2c1OCCN(c1nc(N)nc(C)c1C(C)C)C2] | 4-[7-(3-aminophenyl)-9-methyl-2,3- dihydro-1,4-benzoxazepin-4(5H)-yl]- 6-methyl-5-(1-methylethyl)pyrimidin- 2-amine |
| 70 | 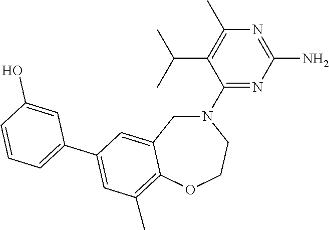 [SMILES: Cc1cc(-c2cccc(O)c2)cc2c1OCCN(c1nc(N)nc(C)c1C(C)C)C2] | 3-{4-[2-amino-6-methyl-5-(1- methylethyl)pyrimidin-4-yl]-9- methyl-2,3,4,5-tetrahydro-1,4- benzoxazepin-7-yl}phenol |
| 71 | 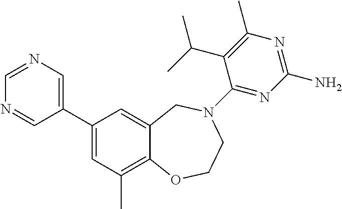 [SMILES: Cc1cc(-c2cncnc2)cc2c1OCCN(c1nc(N)nc(C)c1C(C)C)C2] | 4-methyl-5-(1-methylethyl)-6-(9- methyl-7-pyrimidin-5-yl-2,3-dihydro- 1,4-benzoxazepin-4(5H)-yl)pyrimidin- 2-amine |
| 72 | 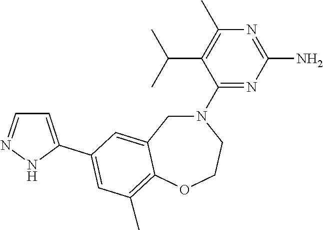 [SMILES: Cc1cc(-c2ccn[nH]2)cc2c1OCCN(c1nc(N)nc(C)c1C(C)C)C2] | 4-methyl-5-(1-methylethyl)-6-[9- methyl-7-(1H-pyrazol-5-yl)-2,3- dihydro-1,4-benzoxazepin-4(5H)- yl]pyrimidin-2-amine |
| 73 | 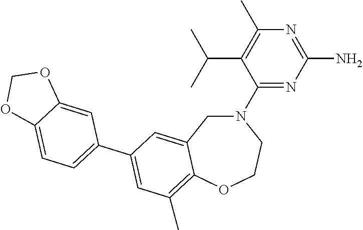 [SMILES: Cc1cc(-c2ccc3c(c2)OCO3)cc2c1OCCN(c1nc(N)nc(C)c1C(C)C)C2] | 4-[7-(1,3-benzodioxol-5-yl)-9-methyl- 2,3-dihydro-1,4-benzoxazepin-4(5H)- yl]-6-methyl-5-(1- methylethyl)pyrimidin-2-amine |
| 74 | 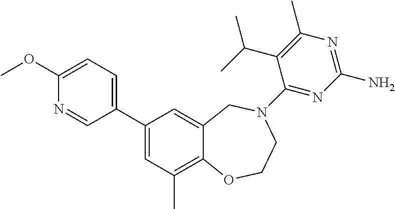 [SMILES: COc1ccc(-c2cc(C)c3c(c2)CN(c2nc(N)nc(C)c2C(C)C)CCO3)cn1] | 4-methyl-5-(1-methylethyl)-6-{9- methyl-7-[6-(methyloxy)pyridin-3-yl]- 2,3-dihydro-1,4-benzoxazepin-4(5H)- yl}pyrimidin-2-amine |
| 75 | 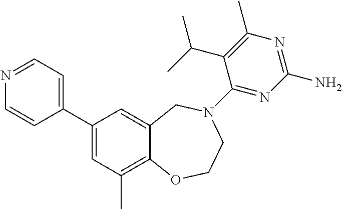 [SMILES: Cc1cc(-c2ccncc2)cc2c1OCCN(c1nc(N)nc(C)c1C(C)C)C2] | 4-methyl-5-(1-methylethyl)-6-(9- methyl-7-pyridin-4-yl-2,3-dihydro- 1,4-benzoxazepin-4(5H)pyrimidin- 2-amine |
| 76 | 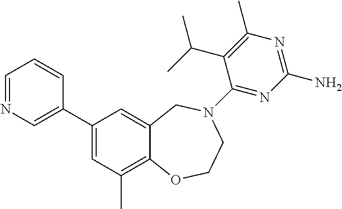 [SMILES: Cc1cc(-c2cccnc2)cc2c1OCCN(c1nc(N)nc(C)c1C(C)C)C2] | 4-methyl-5-(1-methylethyl)-6-(9- methyl-7-pyridin-3-yl-2,3-dihydro- 1,4-benzoxazepin-4(5H)-yl)pyrimidin- 2-amine |
| 77 | 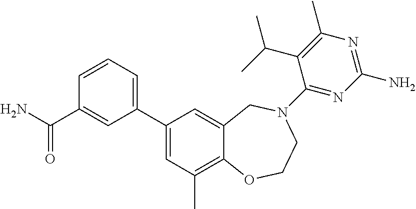 [SMILES: Cc1cc(-c2cccc(C(N)=O)c2)cc2c1OCCN(c1nc(N)nc(C)c1C(C)C)C2] | 3-{4-[2-amino-6-methyl-5-(1- methylethyl)pyrimidin-4-yl]-9- methyl-2,3,4,5-tetrahydro-1,4- benzoxazepin-7-yl}benzamide |
| 78 | 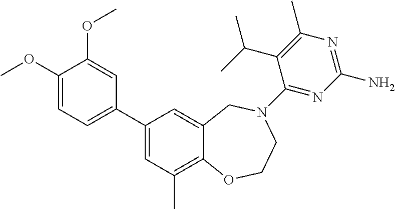 [SMILES: COc1ccc(-c2cc(C)c3c(c2)CN(c2nc(N)nc(C)c2C(C)C)CCO3)cc1OC] | 4-{7-[3,4-bis(methyloxy)phenyl]-9- methyl-2,3-dihydro-1,4-benzoxazepin- 4(5H)-yl}-6-methyl-5-(1- methylethyl)pyrimidin-2-amine |
| 79 | 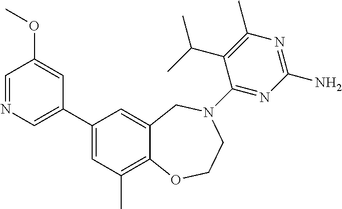 [SMILES: COc1cncc(-c2cc(C)c3c(c2)CN(c2nc(N)nc(C)c2C(C)C)CCO3)c1] | 4-methyl-5-(1-methylethyl)-6-{9- methyl-7-[5-(methyloxy)pyridin-3-yl]- 2,3-dihydro-1,4-benzoxazepin-4(5H)- yl}pyrimidin-2-amine |
| 80 | 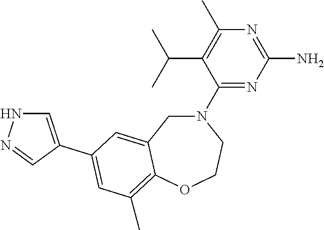 [SMILES: Cc1cc(-c2cn[nH]c2)cc2c1OCCN(c1nc(N)nc(C)c1C(C)C)C2] | 4-methyl-5-(1-methylethyl)-6-[9- methyl-7-(1H-pyrazol-4-yl)-2,3- dihydro-1,4-benzoxazepin-4(5H)- yl]pyrimidin-2-amine |
| 81 | 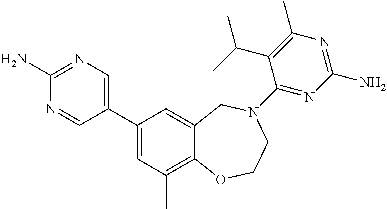 [SMILES: Cc1cc(-c2cnc(N)nc2)cc2c1OCCN(c1nc(N)nc(C)c1C(C)C)C2] | 4-[7-(2-aminopyrimidin-5-yl)-9- methyl-2,3-dihydro-1,4-benzoxazepin- 4(5H)-yl]-6-methyl-5-(1- methylethyl)pyrimidin-2-amine |
| 82 | 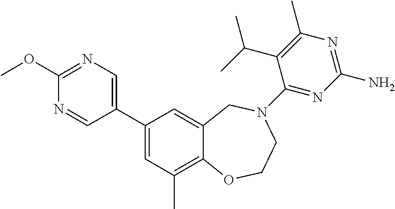 [SMILES: COc1ncc(-c2cc(C)c3c(c2)CN(c2nc(N)nc(C)c2C(C)C)CCO3)cn1] | 4-methyl-5-(1-methylethyl)-6-{9- methyl-7-[2-(methyloxy)pyrimidin-5- yl]-2,3-dihydro-1,4-benzoxazepin- 4(5H)-yl}pyrimidin-2-amine |
| 83 | 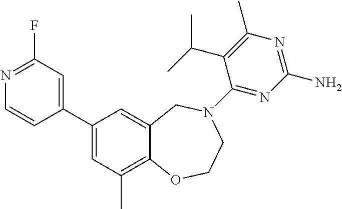 [SMILES: Cc1cc(-c2ccnc(F)c2)cc2c1OCCN(c1nc(N)nc(C)c1C(C)C)C2] | 4-[7-(2-fluoropyridin-4-yl)-9-methyl- 2,3-dihydro-1,4-benzoxazepin-4(5H)- yl]-6-methyl-5-(1- methylethyl)pyrimidin-2-amine |
| 84 | 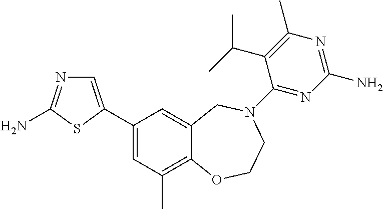 [SMILES: Cc1cc(-c2cnc(N)s2)cc2c1OCCN(c1nc(N)nc(C)c1C(C)C)C2] | 4-[7-(2-amino-1,3-thiazol-5-yl)-9- methyl-2,3-dihydro-1,4-benzoxazepin- 4(5H)-yl]-6-methyl-5-(1- methylethyl)pyrimidin-2-amine |
| 85 | 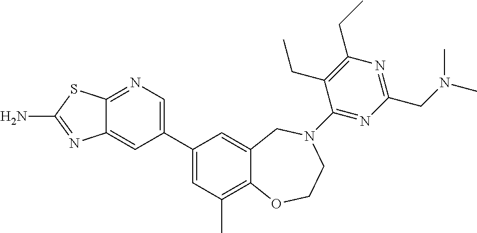 [SMILES: CCc1nc(CN(C)C)nc(N2CCOc3c(C)cc(-c4cnc5sc(N)nc5c4)cc3C2)c1CC] | 6-(4-{2-[(dimethylamino)methyl]-5,6- diethylpyrimidin-4-yl}-9-methyl- 2,3,4,5-tetrahydro-1,4-benzoxazepin- 7-yl)[1,3]thiazolo[5,4-b]pyridin-2- amine |
| 86 | 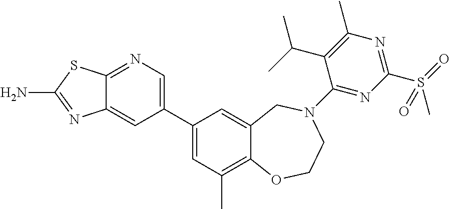 [SMILES: Cc1cc(-c2cnc3sc(N)nc3c2)cc2c1OCCN(c1nc(S(C)(=O)=O)nc(C)c1C(C)C)C2] | 6-{9-methyl-4-[6-methyl-5-(1- methylethyl)-2- (methylsulfonyl)pyrimidin-4-yl]- 2,3,4,5-tetrahydro-1,4-benzoxazepin- 7-yl}[1,3]thiazolo[5,4-b]pyridin-2- amine |
| 87 | 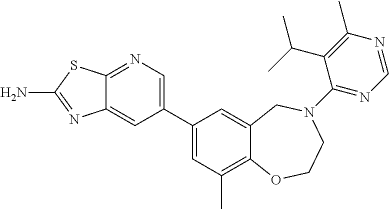 [SMILES: Cc1cc(-c2cnc3sc(N)nc3c2)cc2c1OCCN(c1ncnc(C)c1C(C)C)C2] | 6-{9-methyl-4-[6-methyl-5-(1- methylethyl)pyrimidin-4-yl]-2,3,4,5- tetrahydro-1,4-benzoxazepin-7- yl}[1,3]thiazolo[5,4-b]pyridin-2- amine |
| 88 | 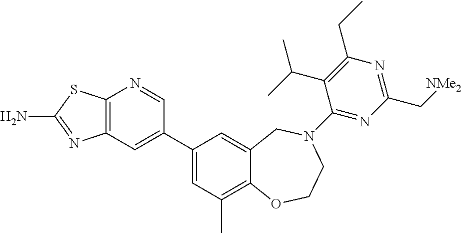 [SMILES: CCc1nc(CN(C)C)nc(N2CCOc3c(C)cc(-c4cnc5sc(N)nc5c4)cc3C2)c1C(C)C] | 6-(4-{2-[(dimethylamino)methyl]-6- ethyl-5-(1-methylethyl)pyrimidin-4- yl}-9-methyl-2,3,4,5-tetrahydro-1,4- benzoxazepin-7-yl)[1,3]thiazolo[5,4- b]pyridin-2-amine |
| 89 | 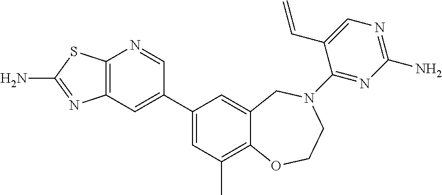 [SMILES: C=Cc1cnc(N)nc1N1CCOc2c(C)cc(-c3cnc4sc(N)nc4c3)cc2C1] | 6-[4-(2-amino-5-ethenylpyrimidin-4- yl)-9-methyl-2,3,4,5-tetrahydro-1,4- benzoxazepin-7-yl][1,3]thiazolo[5,4- b]pyridin-2-amine |
| 90 | 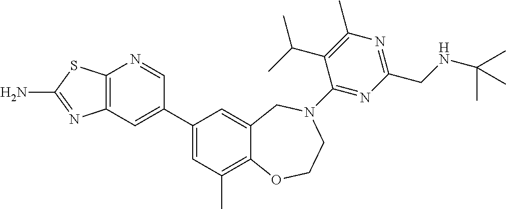 [SMILES: Cc1cc(-c2cnc3sc(N)nc3c2)cc2c1OCCN(c1nc(CNC(C)(C)C)nc(C)c1C(C)C)C2] | 6-{4-[2-{[(1,1- dimethylethyl)amino]methyl}-6- methyl-5-(1-methylethyl)pyrimidin-4- yl]-9-methyl-2,3,4,5-tetrahydro-1,4- benzoxazepin-7-yl}[1,3]thiazolo[5,4- b]pyridin-2-amine |
| 91 | 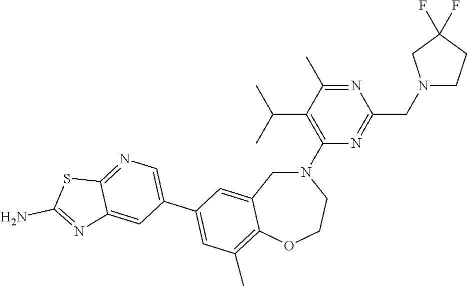 [SMILES: Cc1cc(-c2cnc3sc(N)nc3c2)cc2c1OCCN(c1nc(CN3CCC(F)(F)C3)nc(C)c1C(C)C)C2] | 6-(4-{2-[(3,3-difluoropyrrolidin-1- yl)methyl]-6-methyl-5-(1- methylethyl)pyrimidin-4-yl}-9- methyl-2,3,4,5-tetrahydro-1,4- benzoxazepin-7-yl)[1,3]thiazolo[5,4- b]pyridin-2-amine |
| 92 | 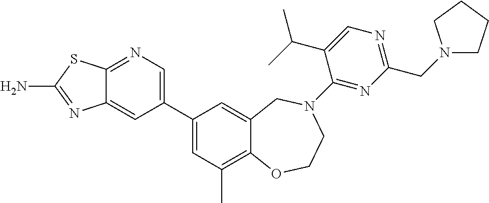 [SMILES: Cc1cc(-c2cnc3sc(N)nc3c2)cc2c1OCCN(c1nc(CN3CCCC3)ncc1C(C)C)C2] | 6-{9-methyl-4-[5-(1-methylethyl)-2- (pyrrolidin-1-ylmethyl)pyrimidin-4- yl]-2,3,4,5-tetrahydro-1,4- benzoxazepin-7-yl}[1,3]thiazolo[5,4- b]pyridin-2-amine |
| 93 | 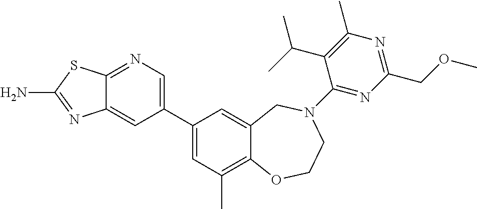 [SMILES: COCc1nc(C)c(C(C)C)c(N2CCOc3c(C)cc(-c4cnc5sc(N)nc5c4)cc3C2)n1] | 6-(9-methyl-4-{6-methyl-5-(1- methylethyl)-2- [(methyloxy)methyl]pyrimidin-4-yl}- 2,3,4,5-tetrahydro-1,4-benzoxazepin- 7-yl)[1,3]thiazolo[5,4-b]pyridin-2- amine |
| 94 | 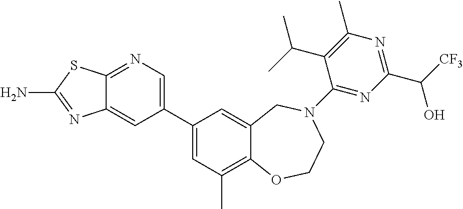 [SMILES: Cc1cc(-c2cnc3sc(N)nc3c2)cc2c1OCCN(c1nc(C(O)C(F)(F)F)nc(C)c1C(C)C)C2] | 1-{4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2-yl}- 2,2,2-trifluoroethanol |
| 95 | 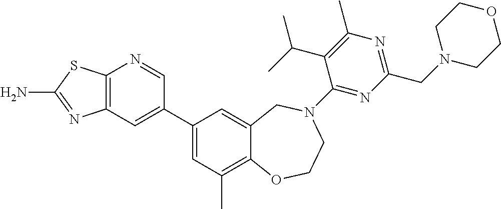 [SMILES: Cc1cc(-c2cnc3sc(N)nc3c2)cc2c1OCCN(c1nc(CN3CCOCC3)nc(C)c1C(C)C)C2] | 6-{9-methyl-4-[6-methyl-5-(1- methylethyl)-2-(morpholin-4- ylmethyl)pyrimidin-4-yl]-2,3,4,5- tetrahydro-1,4-benzoxazepin-7- yl}[1,3]thiazolo[5,4-b]pyridin-2- amine |
| 96 | 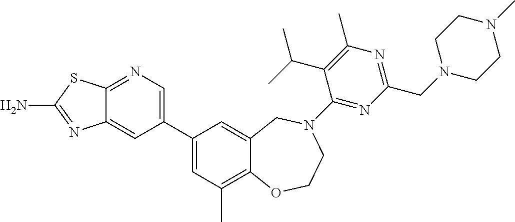 [SMILES: Cc1cc(-c2cnc3sc(N)nc3c2)cc2c1OCCN(c1nc(CN3CCN(C)CC3)nc(C)c1C(C)C)C2] | |
| 97 | 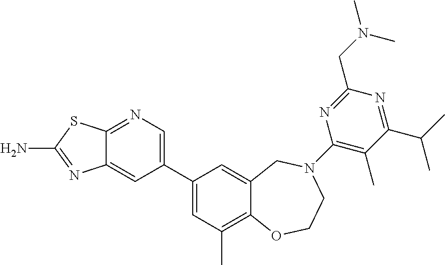 [SMILES: Cc1cc(-c2cnc3sc(N)nc3c2)cc2c1OCCN(c1nc(CN(C)C)nc(C(C)C)c1C)C2] | |
| 98 | 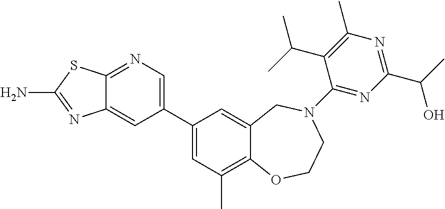 [SMILES: Cc1cc(-c2cnc3sc(N)nc3c2)cc2c1OCCN(c1nc(C(C)O)nc(C)c1C(C)C)C2] | 1-{4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2- yl}ethanol |
| 99 | 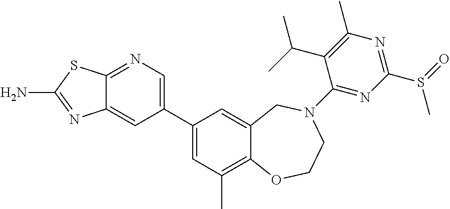 [SMILES: Cc1cc(-c2cnc3sc(N)nc3c2)cc2c1OCCN(c1nc(S(C)=O)nc(C)c1C(C)C)C2] | 6-{9-methyl-4-[6-methyl-5-(1- methylethyl)-2- (methylsulfinyl)pyrimidin-4-yl]- 2,3,4,5-tetrahydro-1,4-benzoxazepin- 7-yl}[1,3]thiazolo[5,4-b]pyridin-2- amine |
| 100 | 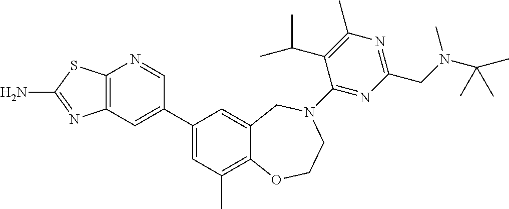 [SMILES: Cc1cc(-c2cnc3sc(N)nc3c2)cc2c1OCCN(c1nc(CN(C)C(C)(C)C)nc(C)c1C(C)C)C2] | 6-{4-[2-{[(1,1- dimethylethyl)(methyl)amino]methyl}- 6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 101 | 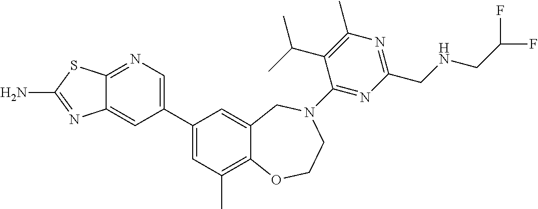 [SMILES: Cc1cc(-c2cnc3sc(N)nc3c2)cc2c1OCCN(c1nc(CNCC(F)F)nc(C)c1C(C)C)C2] | 6-{4-[2-{[(2,2- difluoroethyl)amino]methyl}-6- methyl-5-(1-methylethyl)pyrimidin-4- yl]-9-methyl-2,3,4,5-tetrahydro-1,4- benzoxazepin-7-yl}[1,3]thiazolo[5,4- b]pyridin-2-amine |
| 102 | 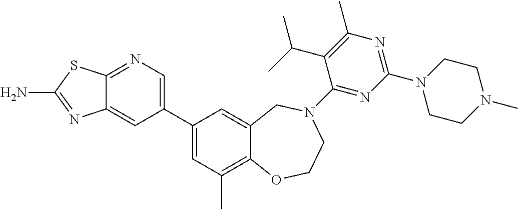 [SMILES: Cc1cc(-c2cnc3sc(N)nc3c2)cc2c1OCCN(c1nc(N3CCN(C)CC3)nc(C)c1C(C)C)C2] | 6-{9-methyl-4-[6-methyl-5-(1- methylethyl)-2-(4-methylpiperazin-1- yl)pyrimidin-4-yl]-2,3,4,5-tetrahydro- 1,4-benzoxazepin-7- yl}[1,3]thiazolo[5,4-b]pyridin-2- amine |
| 103 | 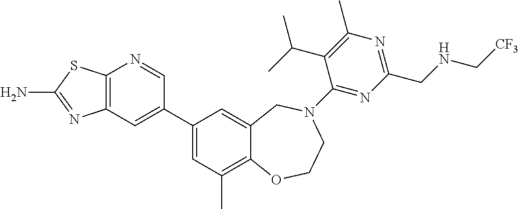 [SMILES: Cc1cc(-c2cnc3sc(N)nc3c2)cc2c1OCCN(c1nc(CNCC(F)(F)F)nc(C)c1C(C)C)C2] | 6-{9-methyl-4-[6-methyl-5-(1- methylethyl)-2-{[(2,2,2- trifluoroethyl)amino]methyl}pyrimidin- 4-yl]-2,3,4,5-tetrahydro-1,4- benzoxazepin-7-yl}[1,3]thiazolo[5,4- b]pyridin-2-amine |
| 104 | 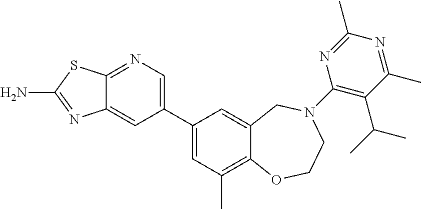 [SMILES: Cc1nc(C)c(C(C)C)c(N2CCOc3c(C)cc(-c4cnc5sc(N)nc5c4)cc3C2)n1] | 6-{4-[2,6-dimethyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 105 | 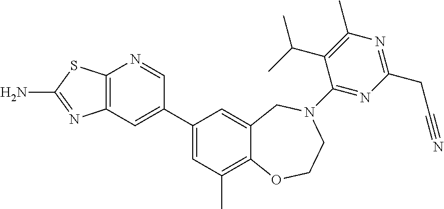 [SMILES: Cc1cc(-c2cnc3sc(N)nc3c2)cc2c1OCCN(c1nc(CC#N)nc(C)c1C(C)C)C2] | {4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2- yl}acetonitrile |
| 106 | 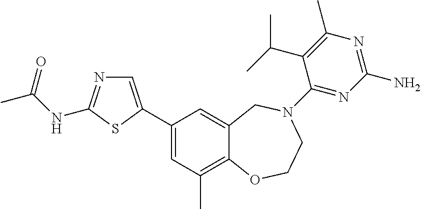 [SMILES: CC(=O)Nc1ncc(-c2cc(C)c3c(c2)CN(c2nc(N)nc(C)c2C(C)C)CCO3)s1] | N-(5-{4-[2-amino-6-methyl-5-(1- methylethyl)pyrimidin-4-yl]-9- methyl-2,3,4,5-tetrahydro-1,4- benzoxazepin-7-yl}-1,3-thiazol-2- yl)acetamide |
| 107 | 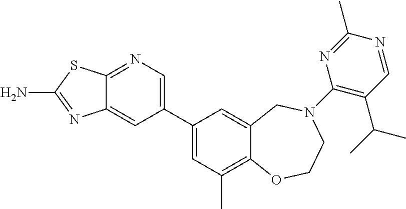 [SMILES: Cc1ncc(C(C)C)c(N2CCOc3c(C)cc(-c4cnc5sc(N)nc5c4)cc3C2)n1] | 6-{9-methyl-4-[2-methyl-5-(1- methylethyl)pyrimidin-4-yl]-2,3,4,5- tetrahyro-1,4-benzoxazepin-7- yl}[1,3]thiazolo[5,4-b]pyridin-2- amine |
| 108 | 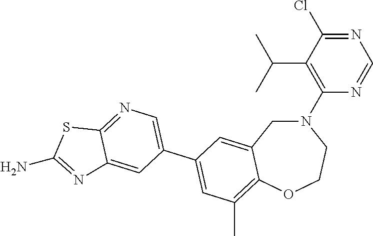 [SMILES: Cc1cc(-c2cnc3sc(N)nc3c2)cc2c1OCCN(c1ncnc(Cl)c1C(C)C)C2] | 6-{4-[6-chloro-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 109 | 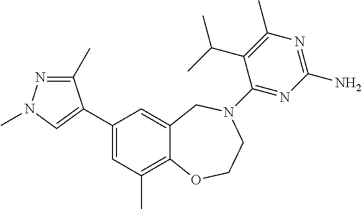 [SMILES: Cc1cc(-c2cn(C)nc2C)cc2c1OCCN(c1nc(N)nc(C)c1C(C)C)C2] | 4-[7-(1,3-dimethyl-1H-pyrazol-4-yl)- 9-methyl-2,3-dihydro-1,4- benzoxazepin-4(5H)-yl]-6-methyl-5- (1-methylethyl)pyrimidin-2-amine |
| 110 | 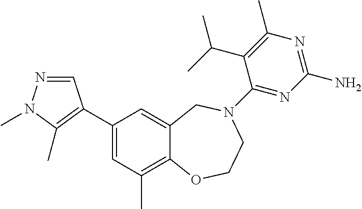 [SMILES: Cc1cc(-c2cnn(C)c2C)cc2c1OCCN(c1nc(N)nc(C)c1C(C)C)C2] | 4-[7-(1,5-dimethyl-1H-pyrazol-4-yl)- 9-methyl-2,3-dihydro-1,4- benzoxazepin-4(5H)-yl]-6-methyl-5- (1-methylethyl)pyrimidin-2-amine |
| 111 | 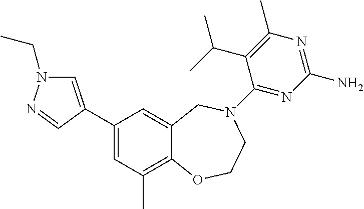 [SMILES: CCn1cc(-c2cc(C)c3c(c2)CN(c2nc(N)nc(C)c2C(C)C)CCO3)cn1] | 4-[7-(1-ethyl-1H-pyrazol-4-yl)-9- methyl-2,3-dihydro-1,4-benzoxazepin- 4(5H)-yl]-6-methyl-5-(1- methylethyl)pyrimidin-2-amine |
| 112 | 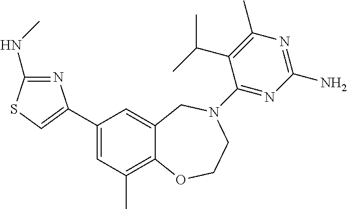 [SMILES: CNc1nc(-c2cc(C)c3c(c2)CN(c2nc(N)nc(C)c2C(C)C)CCO3)cs1] | 4-methyl-5-(1-methylethyl)-6-{9- methyl-7-[2-(methylamino)-1,3- thiazol-4-yl]-2,3-dihydro-1,4- benzoxazepin-4(5H)-yl}pyrimidin-2- amine |
| 113 | 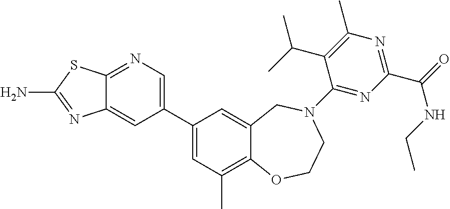 [SMILES: CCNC(=O)c1nc(C)c(C(C)C)c(N2CCOc3c(C)cc(-c4cnc5sc(N)nc5c4)cc3C2)n1] | 4-[7-(2-amino[1,3]thiazolo[5,4- b]pyridin-6-yl)-9-methyl-2,3-dihydro- 1,4-benzoxazepin-4(5H)-yl]-N-ethyl- 6-methyl-5-(1- methylethyl)pyrimidine-2- carboxamide |
| 114 | 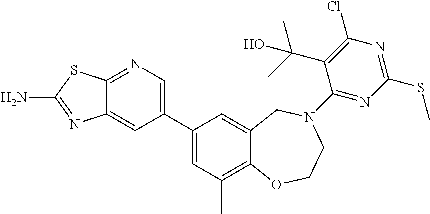 [SMILES: CSc1nc(Cl)c(C(C)(C)O)c(N2CCOc3c(C)cc(-c4cnc5sc(N)nc5c4)cc3C2)n1] | 2-{4-[7-(2-amino[1,3]thiazolo[5,4- b]pyridin-6-yl)-9-methyl-2,3-dihydro- 1,4-benzoxazepin-4(5H)-yl]-6-chloro- 2-(methylthio)pyrimidin-5-yl}propan- 2-ol |
| 115 | 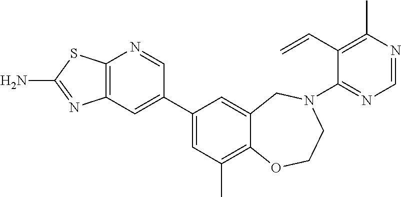 [SMILES: C=Cc1c(C)ncnc1N1CCOc2c(C)cc(-c3cnc4sc(N)nc4c3)cc2C1] | 6-[4-(5-ethenyl-6-methylpyrimidin-4- yl)-9-methyl-2,3,4,5-tetrahydro-1,4- benzoxazepin-7-yl][1,3]thiazolo[5,4- b]pyridin-2-amine |
| 116 | 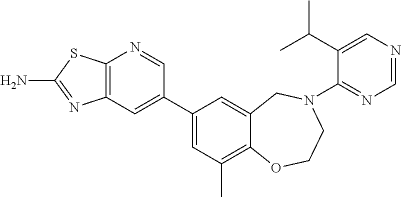 [SMILES: Cc1cc(-c2cnc3sc(N)nc3c2)cc2c1OCCN(c1ncncc1C(C)C)C2] | 6-{9-methyl-4-[5-(1- methylethyl)pyrimidin-4-yl]-2,3,4,5- tetrahydro-1,4-benzoxazepin-7- yl}[1,3]thiazolo[5,4-b]pyridin-2- amine |
| 117 | 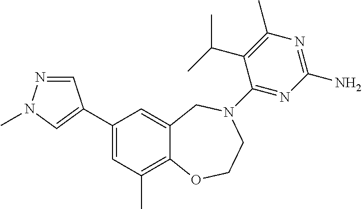 [SMILES: Cc1cc(-c2cnn(C)c2)cc2c1OCCN(c1nc(N)nc(C)c1C(C)C)C2] | 4-methyl-5-(1-methylethyl)-6-[9- methyl-7-(1-methyl-1H-pyrazol-4-yl)- 2,3-dihydro-1,4-benzoxazepin-4(5H)- yl]pyrimidin-2-amine |
| 118 | 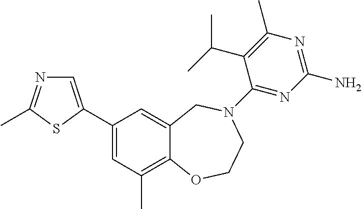 [SMILES: Cc1ncc(-c2cc(C)c3c(c2)CN(c2nc(N)nc(C)c2C(C)C)CCO3)s1] | 4-methyl-5-(1-methylethyl)-6-[9- methyl-7-(2-methyl-1,3-thiazol-5-yl)- 2,3-dihydro-1,4-benzoxazepin-4(5H)- yl]pyrimidin-2-amine |
| 119 | 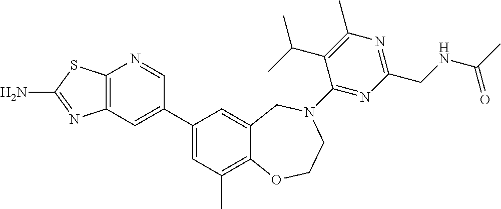 [SMILES: CC(=O)NCc1nc(C)c(C(C)C)c(N2CCOc3c(C)cc(-c4cnc5sc(N)nc5c4)cc3C2)n1] | N-({4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2- yl}methyl)acetamide |
| 120 | 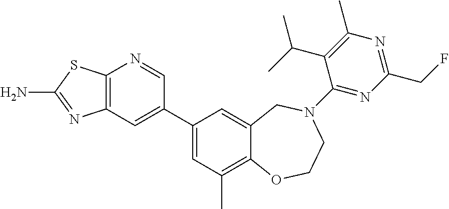 [SMILES: Cc1cc(-c2cnc3sc(N)nc3c2)cc2c1OCCN(c1nc(CF)nc(C)c1C(C)C)C2] | 6-{4-[2-(fluoromethyl)-6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 121 | 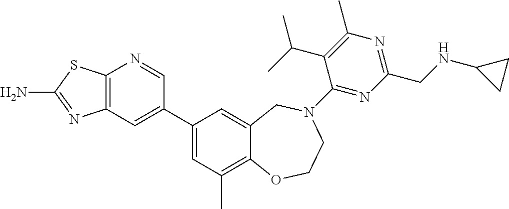 [SMILES: Cc1cc(-c2cnc3sc(N)nc3c2)cc2c1OCCN(c1nc(CNC3CC3)nc(C)c1C(C)C)C2] | 6-(4-{2-[(cyclopropylamino)methyl]- 6-methyl-5-(1-methylethyl)pyrimidin- 4-yl}-9-methyl-2,3,4,5-tetrahydro-1,4- benzoxazepin-7-yl)[1,3]thiazolo[5,4- b]pyridin-2-amine |
| 122 | 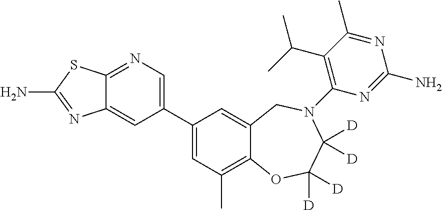 [SMILES: [2H]C1([2H])Oc2c(C)cc(-c3cnc4sc(N)nc4c3)cc2CN(c2nc(N)nc(C)c2C(C)C)C1([2H])[2H]] | 6-{4-[2-amino-6-methyl-5-(1- methylethyl)pyrimidin-4-yl]-2,3,4,5- tetrahydro-1,4-benzoxazepin-7- yl}[1,3]thiazolo[5,4-b]pyridin-2- amine-d_4_ |
| 123 | 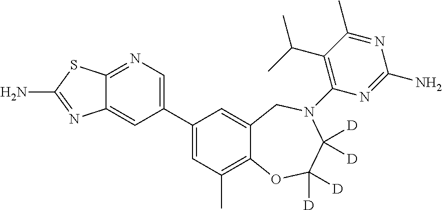 [SMILES: [2H]C1([2H])Oc2c(C)cc(-c3cnc4sc(N)nc4c3)cc2CN(c2nc(N)nc(C)c2C(C)C)C1([2H])[2H]] | 6-{4-[2-amino-6-methyl-5-(1- methylethyl)pyrimidin-4-yl]-2,3,4,5- tetrahydro-1,4-benzoxazepin-7- yl}[1,3]thiazolo[5,4-b]pyridin-2- amine-d_6_ |
| 124 | 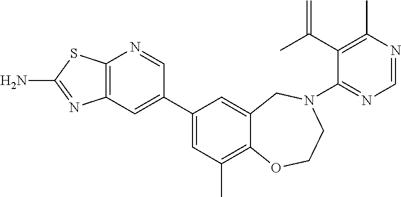 [SMILES: C=C(C)c1c(C)ncnc1N1CCOc2c(C)cc(-c3cnc4sc(N)nc4c3)cc2C1] | 6-{9-methyl-4-[6-methyl-5-(1- methylethenyl)pyrimidin-4-yl]- 2,3,4,5-tetrahydro-1,4-benzoxazepin- 7-yl}[1,3]thiazolo[5,4-b]pyridin-2- amine |
| 125 | 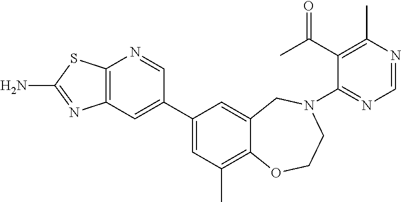 [SMILES: CC(=O)c1c(C)ncnc1N1CCOc2c(C)cc(-c3cnc4sc(N)nc4c3)cc2C1] | 1-{4-[7-(2-amino[1,3]thiazolo[5,4- b]pyridin-6-yl)-9-methyl-2,3-dihydro- 1,4-benzoxazepin-4(5H)-yl]-6- methylpyrimidin-5-yl}ethanone |
| 126 | 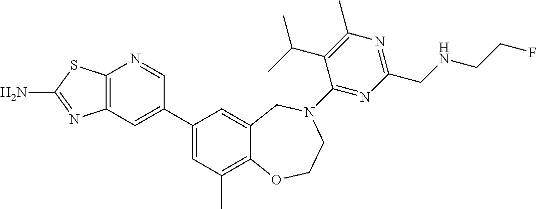 [SMILES: Cc1cc(-c2cnc3sc(N)nc3c2)cc2c1OCCN(c1nc(CNCCF)nc(C)c1C(C)C)C2] | 6-{4-[2-{[(2- fluoroethyl)amino]methyl}-6-methyl- 5-(1-methylethyl)pyrimidin-4-yl]-9- methyl-2,3,4,5-tetrahydro-1,4- benzoxazepin-7-yl}[1,3]thiazolo[5,4- b]pyridin-2-amine |
| 127 | 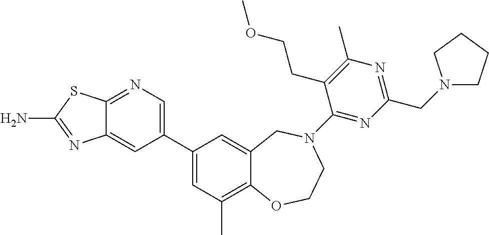 [SMILES: COCCc1c(C)nc(CN2CCCC2)nc1N1CCOc2c(C)cc(-c3cnc4sc(N)nc4c3)cc2C1] | 6-(9-methyl-4-{6-methyl-5-[2- (methyloxy)ethyl]-2-(pyrrolidin-1- ylmethyl)pyrimidin-4-yl}-2,3,4,5- tetrahydro-1,4-benzoxazepin-7- yl)[1,3]thiazolo[5,4-b]pyridin-2-amine |
| 128 | 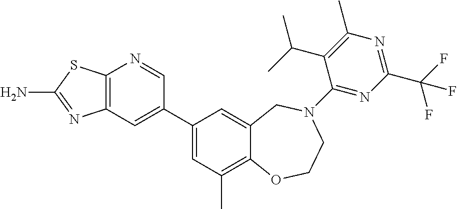 [SMILES: Cc1cc(-c2cnc3sc(N)nc3c2)cc2c1OCCN(c1nc(C(F)(F)F)nc(C)c1C(C)C)C2] | 6-{9-methyl-4-[6-methyl-5-(1- methylethyl)-2- (trifluoromethyl)pyrimidin-4-yl]- 2,3,4,5-tetrahydro-1,4-benzoxazepin- 7-yl}[1,3]thiazolo[5,4-b]pyridin-2- amine |
| 129 | 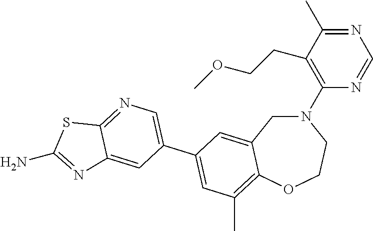 [SMILES: COCCc1c(C)ncnc1N1CCOc2c(C)cc(-c3cnc4sc(N)nc4c3)cc2C1] | 6-(9-methyl-4-{6-methyl-5-[2- (methyloxy)ethyl]pyrimidin-4-yl}- 2,3,4,5-tetrahydrio-1,4-benzoxazepin- 7-yl)[1,3]thiazolo[5,4-b]pyridin-2- amine |
| 130 | 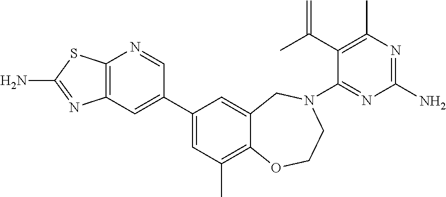 [SMILES: C=C(C)c1c(C)nc(N)nc1N1CCOc2c(C)cc(-c3cnc4sc(N)nc4c3)cc2C1] | 6-{4-[2-amino-6-methyl-5-(1- methylethenyl)pyrimidin-4-yl]-9- methyl-2,3,4,5-tetrahydro-1,4- benzoxazepin-7-yl}[1,3]thiazolo[5,4- b]pyridin-2-amine |
| 131 | 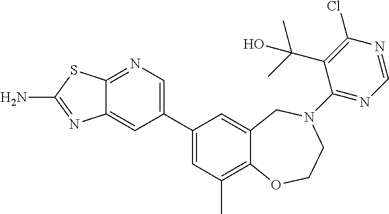 [SMILES: Cc1cc(-c2cnc3sc(N)nc3c2)cc2c1OCCN(c1ncnc(Cl)c1C(C)(C)O)C2] | 2-{4-[7-(2-amino[1,3]thiazolo[5,4- b]pyridin-6-yl)-9-methyl-2,3-dihydro- 1,4-benzoxazepin-4(5H)-yl]-6- chloropyrimidin-5-yl}propan-2-ol |
| 132 | 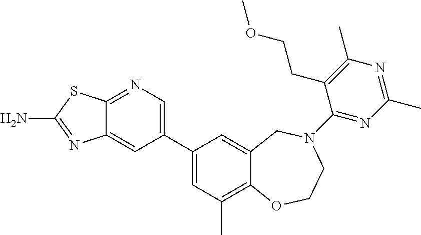 [SMILES: COCCc1c(C)nc(C)nc1N1CCOc2c(C)cc(-c3cnc4sc(N)nc4c3)cc2C1] | 6-(4-{2,6-dimethyl-5-[2- (methyloxy)ethyl]pyrimidin-4-yl}-9- methyl-2,3,4,5-tetrahydro-1,4- benzoxazepin-7-yl)[1,3]thiazolo[5,4- b]pyridin-2-amine |
| 133 | 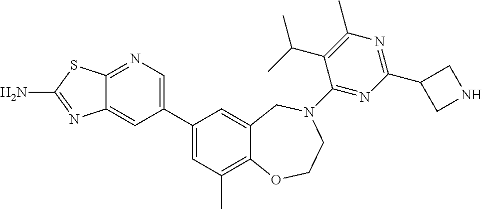 [SMILES: Cc1cc(-c2cnc3sc(N)nc3c2)cc2c1OCCN(c1nc(C3CNC3)nc(C)c1C(C)C)C2] | 6-{4-[2-azetidin-3-yl-6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 134 | 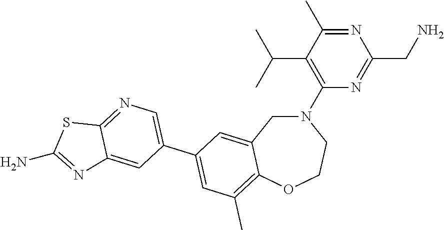 [SMILES: Cc1cc(-c2cnc3sc(N)nc3c2)cc2c1OCCN(c1nc(CN)nc(C)c1C(C)C)C2] | 6-{4-[2-(aminomethyl)-6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 135 | 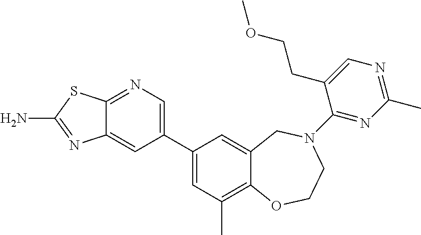 [SMILES: COCCc1cnc(C)nc1N1CCOc2c(C)cc(-c3cnc4sc(N)nc4c3)cc2C1] | 6-(9-methyl-4-{2-methyl-5-[2- (methyloxy)ethyl]pyrimidin-4-yl}- 2,3,4,5-tetrahydro-1,4-benzoxazepin- 7-yl)[1,3]thiazolo[5,4-b]pyridin-2- amine |
| 136 | 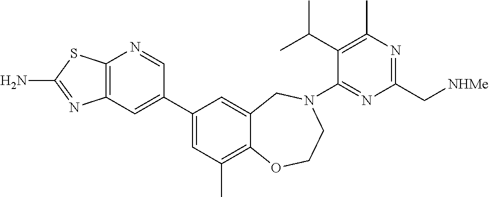 [SMILES: CNCc1nc(C)c(C(C)C)c(N2CCOc3c(C)cc(-c4cnc5sc(N)nc5c4)cc3C2)n1] | 6-(9-methyl-4-{6-methyl-2- [(methylamino)methyl]-5-(1- methylethyl)pyrimidin-4-yl}-2,3,4,5- tetrahydro-1,4-benzoxazepin-7- yl)[1,3]thiazolo[5,4-b]pyridin-2-amine |
| 137 | 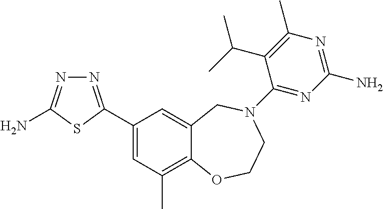 [SMILES: Cc1cc(-c2nnc(N)s2)cc2c1OCCN(c1nc(N)nc(C)c1C(C)C)C2] | 4-[7-(5-amino-1,3,4-thiadiazol-2-yl)- 9-methyl-2,3-dihydro-1,4- benzoxazepin-4(5H)-yl]-6-methyl-5- (1-methylethyl)pyrimidin-2-amine |
| 138 | 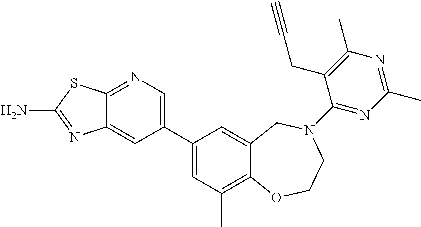 [SMILES: C#CCc1c(C)nc(C)nc1N1CCOc2c(C)cc(-c3cnc4sc(N)nc4c3)cc2C1] | 6-[4-(2,6-dimethyl-5-prop-2-yn-1- ylpyrimidin-4-yl)-9-methyl-2,3,4,5- tetrahydro-1,4-benzoxazepin-7- yl][1,3]thiazolo[5,4-b]pyridin-2-amine |
| 139 | 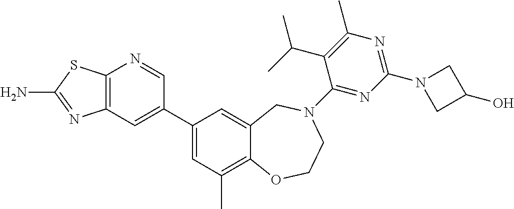 [SMILES: Cc1cc(-c2cnc3sc(N)nc3c2)cc2c1OCCN(c1nc(N3CC(O)C3)nc(C)c1C(C)C)C2] | 1-{4-[7-(2-amino[1,3]thiazolo[5,4- b]pyridin-6-yl)-9-methyl-2,3-dihydro- 1,4-benzoxazepin-4(5H)-yl]-6-methyl- 5-(1-methylethyl)pyrimidin-2- yl}azetidin-3-ol |
| 140 | 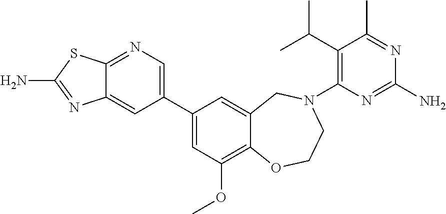 [SMILES: COc1cc(-c2cnc3sc(N)nc3c2)cc2c1OCCN(c1nc(N)nc(C)c1C(C)C)C2] | 6-{4-[2-amino-6-methyl-5-(1- methylethyl)pyrimidin-4-yl]-9- (methyloxy)-2,3,4,5-tetrahydro-1,4- benzoxazepin-7-yl}[1,3]thiazolo[5,4- b]pyridin-2-amine |
| 141 | 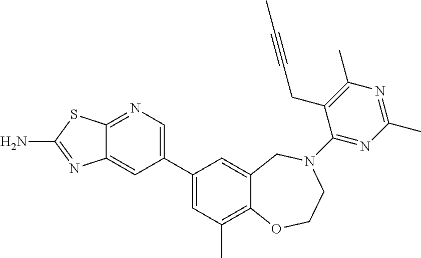 [SMILES: CC#CCc1c(C)nc(C)nc1N1CCOc2c(C)cc(-c3cnc4sc(N)nc4c3)cc2C1] | 6-[4-(5-but-2-yn-1-yl-2,6- dimethylpyrimidin-4-yl)-9-methyl- 2,3,4,5-tetrahydro-1,4-benzoxazepin- 7-yl][1,3]thiazolo[5,4-b]pyridin-2- amine |
| 142 | 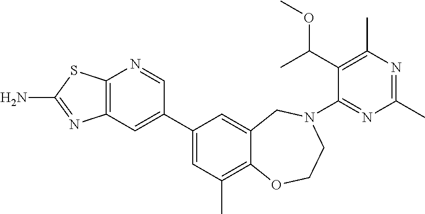 [SMILES: COC(C)c1c(C)nc(C)nc1N1CCOc2c(C)cc(-c3cnc4sc(N)nc4c3)cc2C1] | 6-(4-{2,6-dimethyl-5-[1- (methyloxy)ethyl]pyrimidin-4-yl}-9- methyl-2,3,4,5-tetrahydro-1,4- benzoxazepin-7-yl)[1,3]thiazolo[5,4- b]pyridin-2-amine |
| 143 | 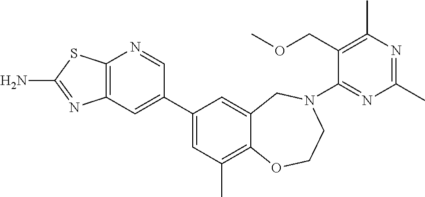 [SMILES: COCc1c(C)nc(C)nc1N1CCOc2c(C)cc(-c3cnc4sc(N)nc4c3)cc2C1] | 6-(4-{2,6-dimethyl-5- [(methyloxy)methyl]pyrimidin-4-yl}- 9-methyl-2,3,4,5-tetrahydro-1,4- benzoxazepin-7-yl)[1,3]thiazolo[5,4- b]pyridin-2-amine |
| 144 | 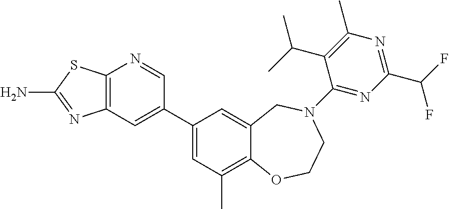 [SMILES: Cc1cc(-c2cnc3sc(N)nc3c2)cc2c1OCCN(c1nc(C(F)F)nc(C)c1C(C)C)C2] | 6-{4-[2-(difluoromethyl)-6-methyl-5- (1-methylethyl)pyrimidin-4-yl]-9- methyl-2,3,4,5-tetrahydro-1,4- benzoxazepin-7-yl}[1,3]thiazolo[5,4- b]pyridin-2-amine |
| 145 | 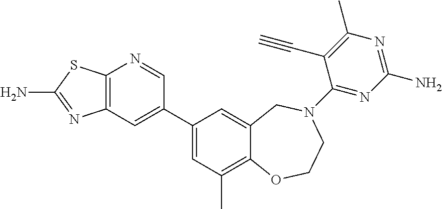 [SMILES: C#Cc1c(C)nc(N)nc1N1CCOc2c(C)cc(-c3cnc4sc(N)nc4c3)cc2C1] | 6-[4-(2-amino-5-ethynyl-6- methylpyrimidin-4-yl)-9-methyl- 2,3,4,5-tetrahydro-1,4-benzoxazepin- 7-yl][1,3]thiazolo[5,4-b]pyridin-2- amine |
| 146 | 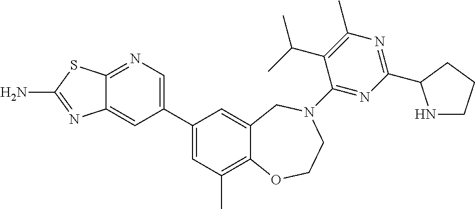 [SMILES: Cc1cc(-c2cnc3sc(N)nc3c2)cc2c1OCCN(c1nc(C3CCCN3)nc(C)c1C(C)C)C2] | 6-{9-methyl-4-[6-methyl-5-(1- methylethyl)-2-pyrrolidin-2- ylpyrimidin-4-yl]-2,3,4,5-tetrahydro- 1,4-benzoxazepin-7- yl}[1,3]thiazolo[5,4-b]pyridin-2- amine |
| 147 | 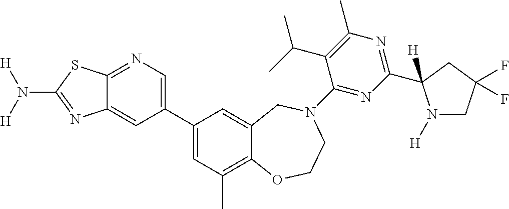 [SMILES: Cc1cc(-c2cnc3sc(N)nc3c2)cc2c1OCCN(c1nc([C@@H]3CC(F)(F)CN3)nc(C)c1C(C)C)C2] | 6-(4-{2-[(2S)-4,4-difluoropyrrolidin- 2-yl]-6-methyl-5-(1- methylethyl)pyrimidin-4-yl}-9- methyl-2,3,4,5-tetrahydro-1,4- benzoxazepin-7-yl)[1,3]thiazolo[5,4- b]pyridin-2-amine |
| 148 | 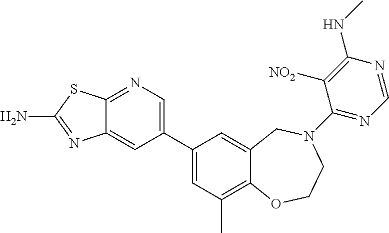 [SMILES: CNc1ncnc(N2CCOc3c(C)cc(-c4cnc5sc(N)nc5c4)cc3C2)c1[N+](=O)[O-]] | 6-{9-methyl-4-[6-(methylamino)-5- nitropyrimidin-4-yl]-2,3,4,5- tetrahydro-1,4-benzoxazepin-7- yl}[1,3]thiazolo[5,4-b]pyridin-2- amine |
| 149 | 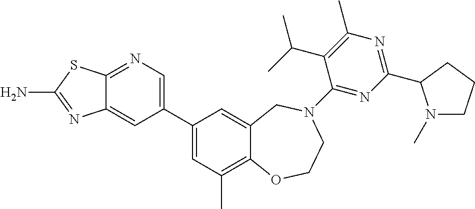 [SMILES: Cc1cc(-c2cnc3sc(N)nc3c2)cc2c1OCCN(c1nc(C3CCCN3C)nc(C)c1C(C)C)C2] | 6-{9-methyl-4-[6-methyl-5-(1- methylethyl)-2-(1-methylpyrrolidin-2- yl)pyrimidin-4-yl]-2,3,4,5-tetrahydro- 1,4-benzoxazepin-7- yl}[1,3]thiazolo[5,4-b]pyridin-2- amine |
| 150 | 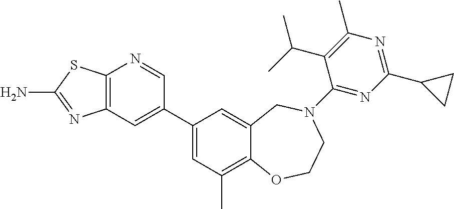 [SMILES: Cc1cc(-c2cnc3sc(N)nc3c2)cc2c1OCCN(c1nc(C3CC3)nc(C)c1C(C)C)C2] | 6-{4-[2-cyclopropyl-6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |
| 151 | 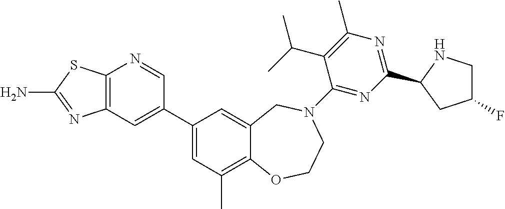 [SMILES: Cc1cc(-c2cnc3sc(N)nc3c2)cc2c1OCCN(c1nc([C@@H]3C[C@@H](F)CN3)nc(C)c1C(C)C)C2] | 6-(4-{2-[(2S,4R)-4-fluoropyrrolidin-2- yl]-6-methyl-5-(1- methylethyl)pyrimidin-4-yl}-9- methyl-2,3,4,5-tetrahydro-1,4- benzoazepin-7-yl)[1,3]thiazolo[5,4- b]pyridin-2-amine |
| 152 | 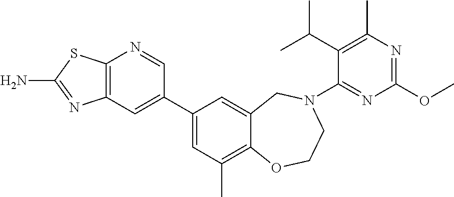 [SMILES: COc1nc(C)c(C(C)C)c(N2CCOc3c(C)cc(-c4cnc5sc(N)nc5c4)cc3C2)n1] | 6-{9-methyl-4-[6-methyl-5-(1- methylethyl)-2-(methyloxy)pyrimidin- 4-yl]-2,3,4,5-tetrahydro-1,4- benzoxazepin-7-yl}[1,3]thiazolo[5,4- b]pyridin-2-amine |
| 153 | 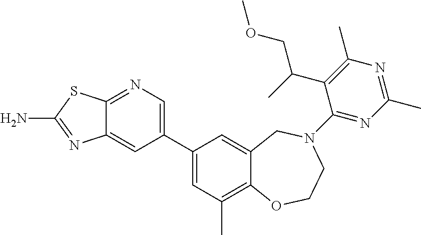 [SMILES: COCC(C)c1c(C)nc(C)nc1N1CCOc2c(C)cc(-c3cnc4sc(N)nc4c3)cc2C1] | 6-(4-{2,6-dimethyl-5-[1-methyl-2- (methyloxy)ethyl]pyrimidin-4-yl}-9- methyl-2,3,4,5-tetrahydro-1,4- benzoxazepin-7-yl)[1,3]thiazolo[5,4- b]pyridin-2-amine |
| 154 | 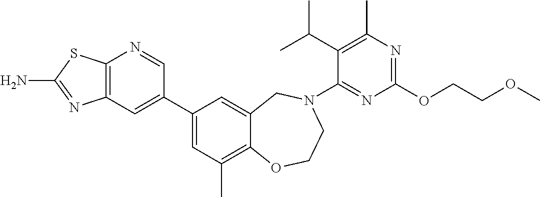 [SMILES: COCCOc1nc(C)c(C(C)C)c(N2CCOc3c(C)cc(-c4cnc5sc(N)nc5c4)cc3C2)n1] | 6-{9-methyl-4-[6-methyl-5-(1- methylethyl)-2-{[2- (methyloxy)ethyl]oxy}pyrimidin-4- yl]-2,3,4,5-tetrahydro-1,4- benzoxazepin-7-yl}[1,3]thiazolo[5,4- b]pyridin-2-amine |
| 155 | 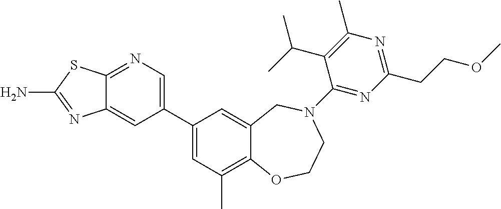 [SMILES: COCCc1nc(C)c(C(C)C)c(N2CCOc3c(C)cc(-c4cnc5sc(N)nc5c4)cc3C2)n1] | 6-(9-methyl-4-{6-methyl-5-(1- methylethyl)-2-[2- (methyloxy)ethyl]pyrimidin-4-yl}- 2,3,4,5-tetrahydro-1,4-benzoxazepin- 7-yl)[1,3]thiazolo[5,4-b]pyridin-2- amine |
| 156 | 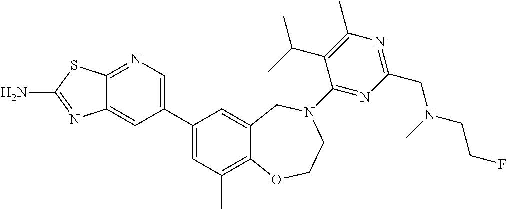 [SMILES: Cc1cc(-c2cnc3sc(N)nc3c2)cc2c1OCCN(c1nc(CN(C)CCF)nc(C)c1C(C)C)C2] | 6-{4-[2-{[(2- fluoroethyl)(methyl)amino]methyl}-6- methyl-5-(1-methylethyl)pyrimidin-4- yl]-9-methyl-2,3,4,5-tetrahydro-1,4- benzoxazepin-7-yl}[1,3]thiazolo[5,4- b]pyridin-2-amine |
| 157 | 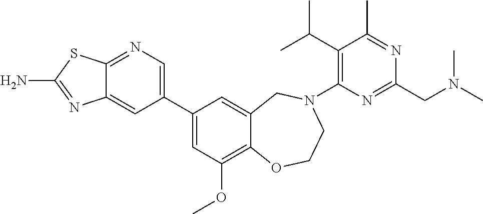 [SMILES: COc1cc(-c2cnc3sc(N)nc3c2)cc2c1OCCN(c1nc(CN(C)C)nc(C)c1C(C)C)C2] | 6-[4-{2-[(dimethylamino)methyl]-6- methyl-5-(1-methylethyl)pyrimidin-4- yl}-9-(methyloxy)-2,3,4,5-tetrahydro- 1,4-benzoxazepin-7- yl][1,3]thiazolo[5,4-b]pyridin-2-amine |
| 158 | 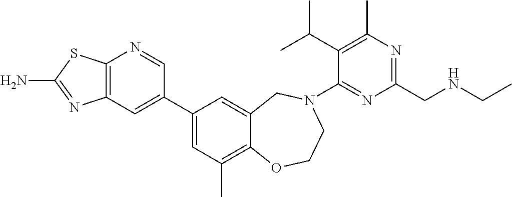 [SMILES: CCNCc1nc(C)c(C(C)C)c(N2CCOc3c(C)cc(-c4cnc5sc(N)nc5c4)cc3C2)n1] | 6-(4-{2-[(ethylamino)methyl]-6- methyl-5-(1-methylethyl)pyrimidin-4- yl}-9-methyl-2,3,4,5-tetrahydro-1,4- benzoxazepin-7-yl)[1,3]thiazolo[5,4- b]pyridin-2-amine |
| 159 | 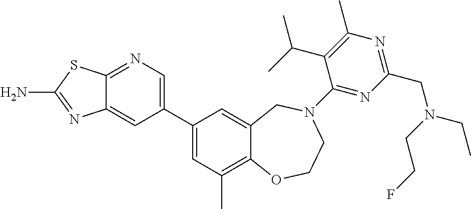 [SMILES: CCN(CCF)Cc1nc(C)c(C(C)C)c(N2CCOc3c(C)cc(-c4cnc5sc(N)nc5c4)cc3C2)n1] | 6-{4-[2-{[ethyl(2- fluoroethyl)amino]methyl}-6-methyl- 5-(1-methylethyl)pyrimidin-4-yl]-9- methyl-2,3,4,5-tetrahydro-1,4- benzoxazepin-7-yl}[1,3]thiazolo[5,4- b]pyridin-2-amine |
| 160 | 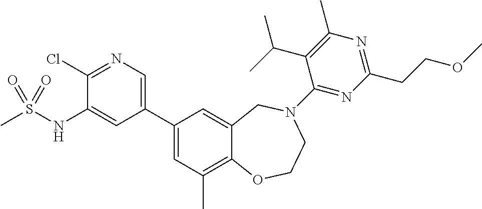 | N-[2-chloro-5-(9-methyl-4-{6-methyl- 5-(1-methylethyl)-2-[2- (methyloxy)ethyl]pyrimidin-4-yl}- 2,3,4,5-tetrahydro-1,4-benzoxazepin- 7-yl)pyridin-3-yl]methanesulfonamide |
| 161 | 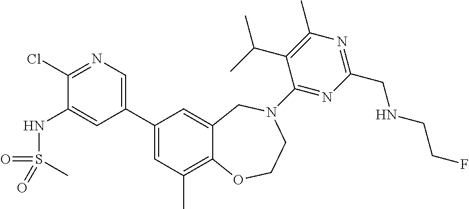 [SMILES: Cc1cc(-c2cnc(Cl)c(NS(C)(=O)=O)c2)cc2c1OCCN(c1nc(CNCCF)nc(C)c1C(C)C)C2] | N-(2-chloro-5-{4-[2-{[(2- fluoroethyl)amino]methyl}-6-methyl- 5-(1-methylethyl)pyrimidin-4-yl]-9- methyl-2,3,4,5-tetrahydro-1,4- benzoxazepin-7-yl}pyridin-3- yl)methanesulfonamide |
| 162 | 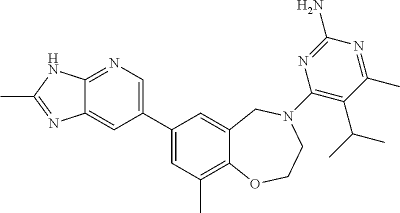 | 4-methyl-5-(1-methylethyl)-6-[9- methyl-7-(2-methyl-3H-imidazo[4,5- b]pyridin-6-yl)-2,3-dihydro-1,4- benzoxazepin-4(5H)-yl]pyrimidin-2- amine |
| 163 | 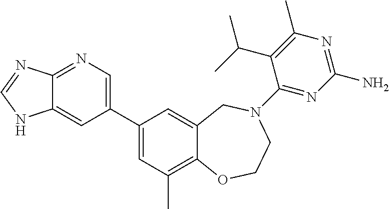 [SMILES: Cc1cc(-c2cnc3nc[nH]c3c2)cc2c1OCCN(c1nc(N)nc(C)c1C(C)C)C2] | 4-[7-(1H-imidazo[4,5-b]pyridin-6-yl)- 9-methyl-2,3-dihydro-1,4- benzoxazepin-4(5H)-yl]-6-methyl-5- (1-methylethyl)pyrimidin-2-amine |
| 164 | 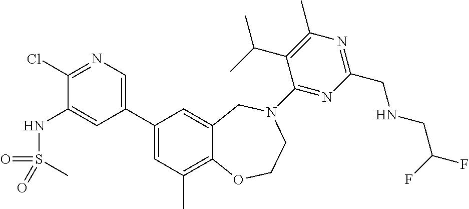 [SMILES: Cc1cc(-c2cnc(Cl)c(NS(C)(=O)=O)c2)cc2c1OCCN(c1nc(CNCC(F)F)nc(C)c1C(C)C)C2] | N-(2-chloro-5-{4-[2-{[(2,2- difluoroethyl)amino]methyl}-6- methyl-5-(1-methylethyl)pyrimidin-4- yl]-9-methyl-2,3,4,5-tetrahydro-1,4- benzoxazepin-7-yl}pyridin-3- yl)methanesulfonamide |
| 165 | 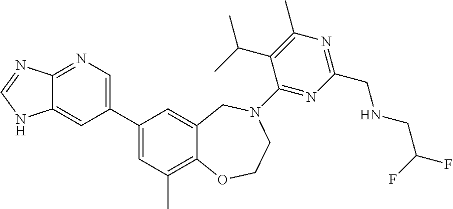 [SMILES: Cc1cc(-c2cnc3nc[nH]c3c2)cc2c1OCCN(c1nc(CNCC(F)F)nc(C)c1C(C)C)C2] | 2,2-difluoro-N-({4-[7-(1H- imidazo[4,5-b]pyridin-6-yl)-9-methyl- 2,3-dihydro-1,4-benzoxazepin-4(5H)- yl]-6-methyl-5-(1- methylethyl)pyrimidin-2- yl}methyl)ethanamine |
| 166 | 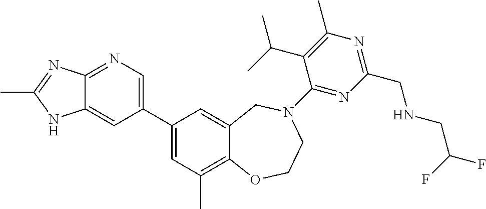 [SMILES: Cc1nc2ncc(-c3cc(C)c4c(c3)CN(c3nc(CNCC(F)F)nc(C)c3C(C)C)CCO4)cc2[nH]1] | 2,2-difluoro-N-({4-methyl-5-(1- methylethyl)-6-[9-methyl-7-(2- methyl-1H-imidazo[4,5-b]pyridin-6- yl)-2,3-dihydro-1,4-benzoxazepin- 4(5H)-yl]pyrimidin-2- yl}methyl)ethanamine |
| 167 | 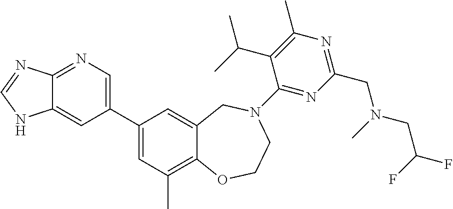 [SMILES: Cc1cc(-c2cnc3nc[nH]c3c2)cc2c1OCCN(c1nc(CN(C)CC(F)F)nc(C)c1C(C)C)C2] | 2,2-difluoro-N-({4-[7-(1H- imidazo[4,5-b]pyridin-6-yl)-9-methyl- 2,3-dihydro-1,4-benzoxazepin-4(5H)- yl]-6-methyl-5-(1- methylethyl)pyrimidin-2-yl}methyl)- N-methylethanamine |
| 168 | 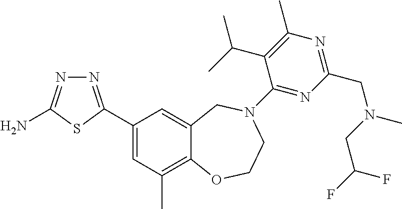 [SMILES: Cc1cc(-c2nnc(N)s2)cc2c1OCCN(c1nc(CN(C)CC(F)F)nc(C)c1C(C)C)C2] | 5-{4-[2-{[(2,2- difluoroethyl)(methyl)amino]methyl}- 6-methyl-5-(1-methylethyl)pyrimidin- 4-yl]-9-methyl-2,3,4,5-tetrahydro-1,4- benzoxazepin-7-yl}-1,3,4-thiadiazol- 2-amine |
| 169 | 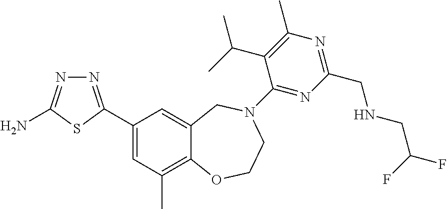 [SMILES: Cc1cc(-c2nnc(N)s2)cc2c1OCCN(c1nc(CNCC(F)F)nc(C)c1C(C)C)C2] | 5-{4-[2-{[(2,2- difluoroethyl)amino]methyl}-6- methyl-5-(1-methylethyl)pyrimidin-4- yl]-9-methyl-2,3,4,5-tetrahydro-1,4- benzoxazepin-7-yl}-1,3,4-thiadiazol- 2-amine |
| 170 | 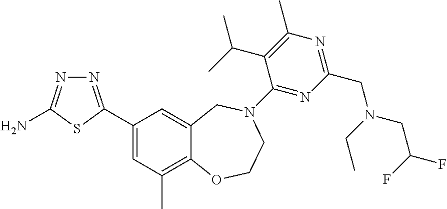 [SMILES: CCN(Cc1nc(C)c(C(C)C)c(N2CCOc3c(C)cc(-c4nnc(N)s4)cc3C2)n1)CC(F)F] | 5-{4-[2-{[(2,2- difluoroethyl)(ethyl)amino]methyl}-6- methyl-5-(1-methylethyl)pyrimidin-4- yl]-9-methyl-2,3,4,5-tetrahydro-1,4- benzoxazepin-7-yl}-1,3,4-thiadiazol- 2-amine |
| 171 | 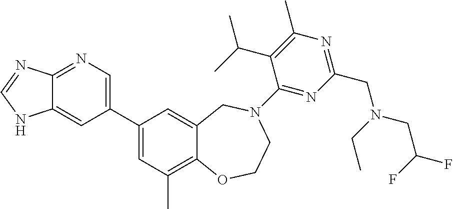 [SMILES: CCN(Cc1nc(C)c(C(C)C)c(N2CCOc3c(C)cc(-c4cnc5nc[nH]c5c4)cc3C2)n1)CC(F)F] | N-ethyl-2,2-difluoro-N-({4-[7-(1H- imidazo[4,5-b]pyridin-6-yl)-9-methyl- 2,3-dihydro-1,4-benzoxazepin-4(5H)- yl]-6-methyl-5-(1- methylethyl)pyrimidin-2- yl}methyl)ethanamine |
| 172 | 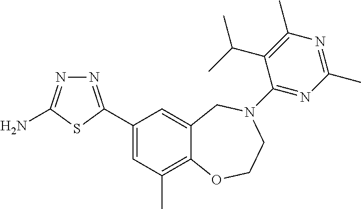 [SMILES: Cc1nc(C)c(C(C)C)c(N2CCOc3c(C)cc(-c4nnc(N)s4)cc3C2)n1] | 5-{4-[2,6-dimethyl-5-(1- methylethyl)pyrimidin-4-yl]-9- methyl-2,3,4,5-tetrahydro-1,4- benzoxazepin-7-yl}-1,3,4-thiadiazol- 2-amine |
| 173 | 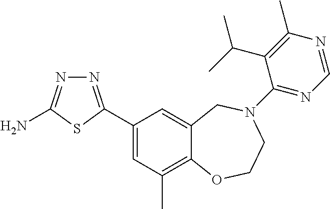 [SMILES: Cc1cc(-c2nnc(N)s2)cc2c1OCCN(c1ncnc(C)c1C(C)C)C2] | 5-{9-methyl-4-[6-methyl-5-(1- methylethyl)pyrimidin-4-yl]-2,3,4,5- tetrahydro-1,4-benzoxazepin-7-yl}- 1,3,4-thiadiazol-2-amine |
| 174 | 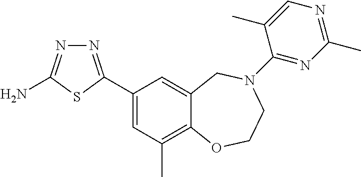 [SMILES: Cc1ncc(C)c(N2CCOc3c(C)cc(-c4nnc(N)s4)cc3C2)n1] | 5-[4-(2,5-dimethylpyrimidin-4-yl)-9- methyl-2,3,4,5-tetrahydro-1,4- benzoxazepin-7-yl]-1,3,4-thiadiazol-2- amine |
| 175 | 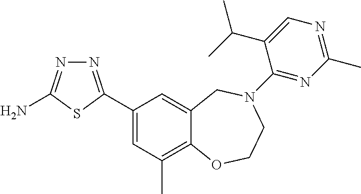 [SMILES: Cc1ncc(C(C)C)c(N2CCOc3c(C)cc(-c4nnc(N)s4)cc3C2)n1] | 5-{9-methyl-4-[2-methyl-5-(1- methylethyl)pyrimidin-4-yl]-2,3,4,5- tetrahydro-1,4-benzoxazepin-7-yl}- 1,3,4-thiadiazol-2-amine |
| 176 | 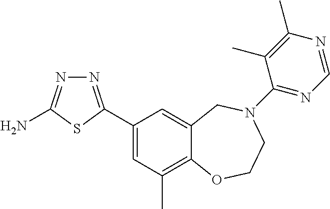 [SMILES: Cc1cc(-c2nnc(N)s2)cc2c1OCCN(c1ncnc(C)c1C)C2] | 5-[4-(5,6-dimethylpyrimidin-4-yl)-9- methyl-2,3,4,5-tetrahydro-1,4- benzoxazepin-7-yl]-1,3,4-thiadiazol-2- amine |
| 177 | 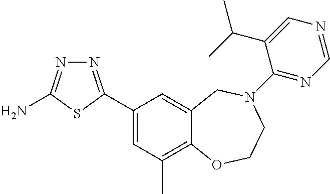 [SMILES: Cc1cc(-c2nnc(N)s2)cc2c1OCCN(c1ncncc1C(C)C)C2] | 5-{9-methyl-4-[5-(1- methylethyl)pyrimidin-4-yl]-2,3,4,5- tetrahydro-1,4-benzoxazepin-7-yl}- 1,3,4-thiadiazol-2-amine |
| 178 | 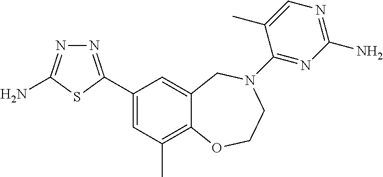 [SMILES: Cc1cnc(N)nc1N1CCOc2c(C)cc(-c3nnc(N)s3)cc2C1] | 4-[7-(5-amino-1,3,4-thiadiazol-2-yl)- 9-methyl-2,3-dihydro-1,4- benzoxazepin-4(5H)-yl]-5- methylpyrimidin-2-amine |
| 179 | 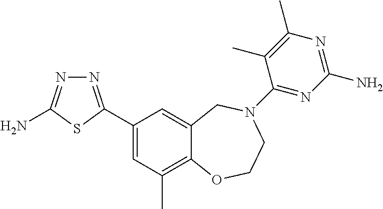 [SMILES: Cc1cc(-c2nnc(N)s2)cc2c1OCCN(c1nc(N)nc(C)c1C)C2] | 4-[7-(5-amino-1,3,4-thiadiazol-2-yl)- 9-methyl-2,3-dihydro-1,4- benzoxazepin-4(5H)-yl]-5,6- dimethylpyrimidin-2-amine |
| 180 | 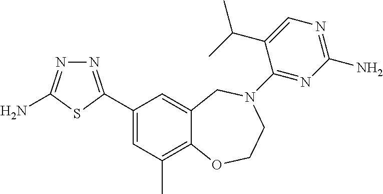 [SMILES: Cc1cc(-c2nnc(N)s2)cc2c1OCCN(c1nc(N)ncc1C(C)C)C2] | 4-[7-(5-amino-1,3,4-thiadiazol-2-yl)- 9-methyl-2,3-dihydro-1,4- benzoxazepin-4(5H)-yl]-5-(1- methylethyl)pyrimidin-2-amine |
| 181 | 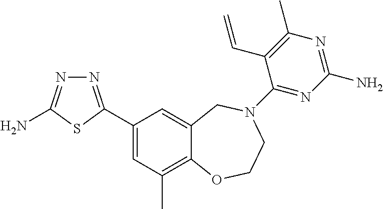 [SMILES: C=Cc1c(C)nc(N)nc1N1CCOc2c(C)cc(-c3nnc(N)s3)cc2C1] | 4-[7-(5-amino-1,3,4-thiadiazol-2-yl)- 9-methyl-2,3-dihydro-1,4- benzoxazepin-4(5H)-yl]-5-ethenyl-6- methylpyrimidin-2-amine |
| 182 | 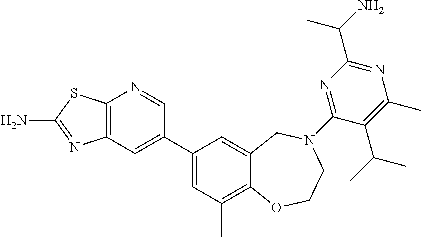 [SMILES: Cc1cc(-c2cnc3sc(N)nc3c2)cc2c1OCCN(c1nc(C(C)N)nc(C)c1C(C)C)C2] | 6-{4-[2-(1-aminoethyl)-6-methyl-5-(1- methylethyl)pyrimidin-4-yl]-9- methyl-2,3,4,5-tetrahydro-1,4- benzoxazepin-7-yl}[1,3]thiazolo[5,4- b]pyridin-2-amine |

In other embodiments, the compounds of the invention include the compounds depicted, below:

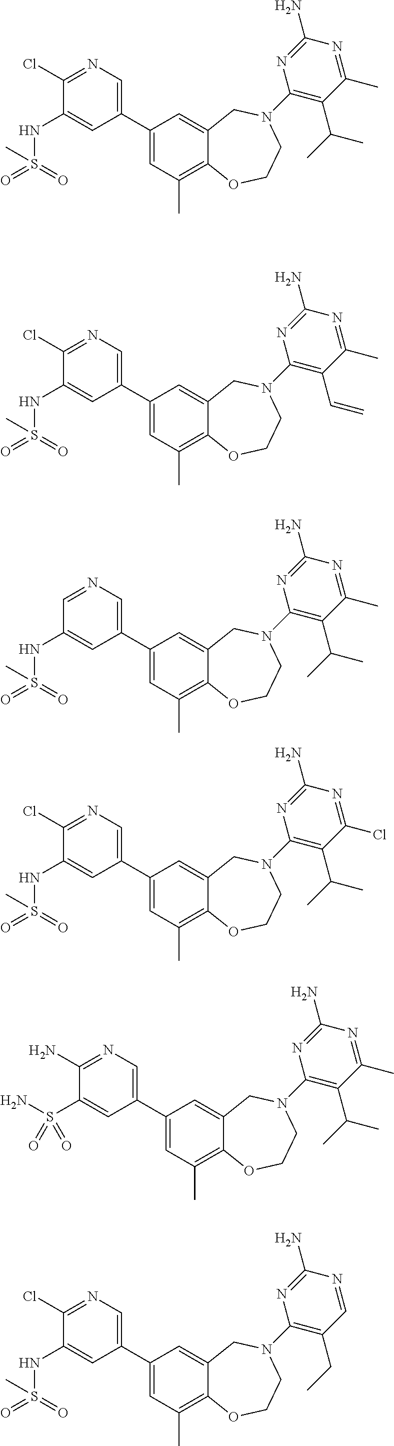 [SMILES: Cc1cc(-c2cnc(Cl)c(NS(C)(=O)=O)c2)cc2c1OCCN(c1nc(N)nc(C)c1C(C)C)C2 ; C=Cc1c(C)nc(N)nc1N1CCOc2c(C)cc(-c3cnc(Cl)c(NS(C)(=O)=O)c3)cc2C1 ; Cc1cc(-c2cncc(NS(C)(=O)=O)c2)cc2c1OCCN(c1nc(N)nc(C)c1C(C)C)C2 ; Cc1cc(-c2cnc(Cl)c(NS(C)(=O)=O)c2)cc2c1OCCN(c1nc(N)nc(Cl)c1C(C)C)C2 ; Cc1cc(-c2cnc(N)c(S(N)(=O)=O)c2)cc2c1OCCN(c1nc(N)nc(C)c1C(C)C)C2 ; CCc1cnc(N)nc1N1CCOc2c(C)cc(-c3cnc(Cl)c(NS(C)(=O)=O)c3)cc2C1]

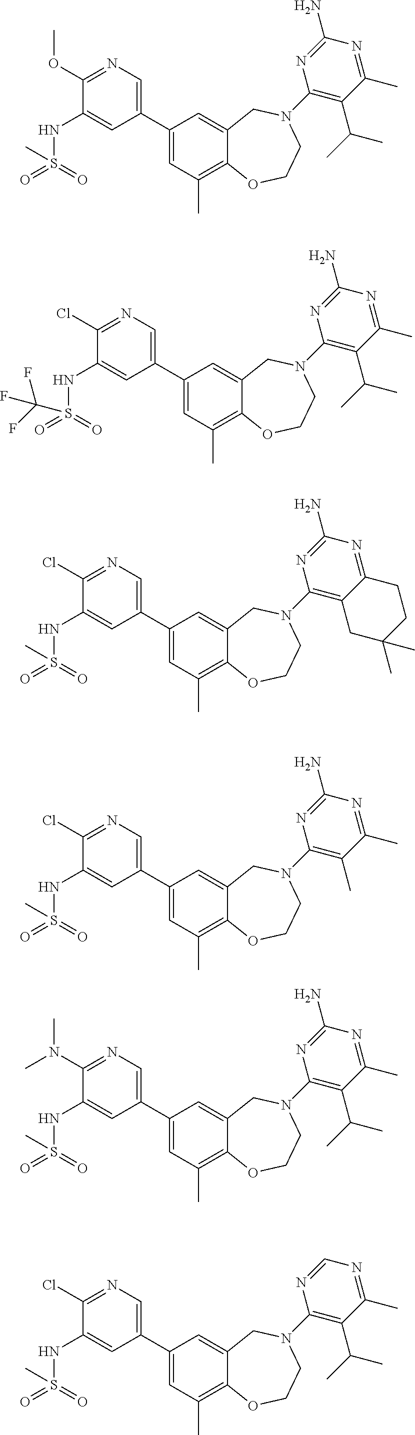 [SMILES: COc1ncc(-c2cc(C)c3c(c2)CN(c2nc(N)nc(C)c2C(C)C)CCO3)cc1NS(C)(=O)=O ; Cc1cc(-c2cnc(Cl)c(NS(=O)(=O)C(F)(F)F)c2)cc2c1OCCN(c1nc(N)nc(C)c1C(C)C)C2 ; Cc1cc(-c2cnc(Cl)c(NS(C)(=O)=O)c2)cc2c1OCCN(c1nc(N)nc3c1CC(C)(C)CC3)C2 ; Cc1cc(-c2cnc(Cl)c(NS(C)(=O)=O)c2)cc2c1OCCN(c1nc(N)nc(C)c1C)C2 ; Cc1cc(-c2cnc(N(C)C)c(NS(C)(=O)=O)c2)cc2c1OCCN(c1nc(N)nc(C)c1C(C)C)C2 ; Cc1cc(-c2cnc(Cl)c(NS(C)(=O)=O)c2)cc2c1OCCN(c1ncnc(C)c1C(C)C)C2]

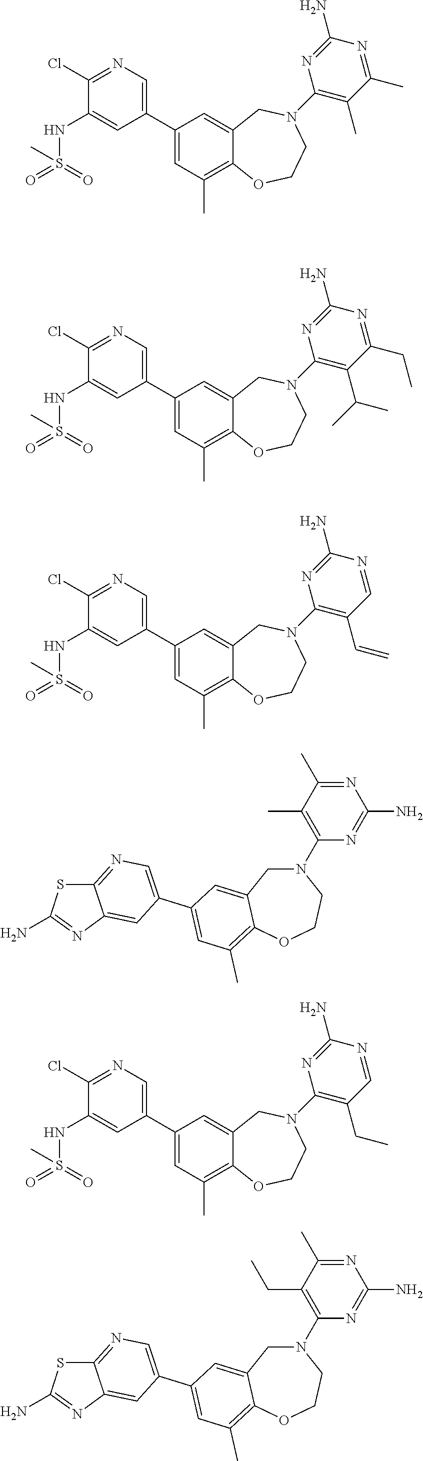 [SMILES: Cc1cc(-c2cnc(Cl)c(NS(C)(=O)=O)c2)cc2c1OCCN(c1nc(N)nc(C)c1C)C2 ; CCc1nc(N)nc(N2CCOc3c(C)cc(-c4cnc(Cl)c(NS(C)(=O)=O)c4)cc3C2)c1C(C)C ; C=Cc1cnc(N)nc1N1CCOc2c(C)cc(-c3cnc(Cl)c(NS(C)(=O)=O)c3)cc2C1 ; Cc1cc(-c2cnc3sc(N)nc3c2)cc2c1OCCN(c1nc(N)nc(C)c1C)C2 ; CCc1cnc(N)nc1N1CCOc2c(C)cc(-c3cnc(Cl)c(NS(C)(=O)=O)c3)cc2C1 ; CCc1c(C)nc(N)nc1N1CCOc2c(C)cc(-c3cnc4sc(N)nc4c3)cc2C1]

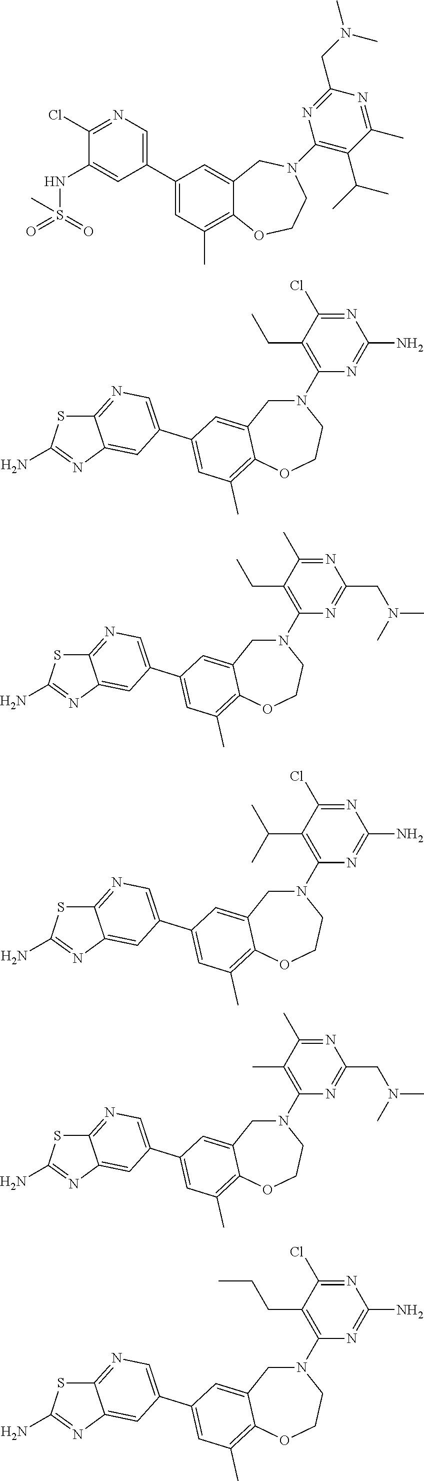

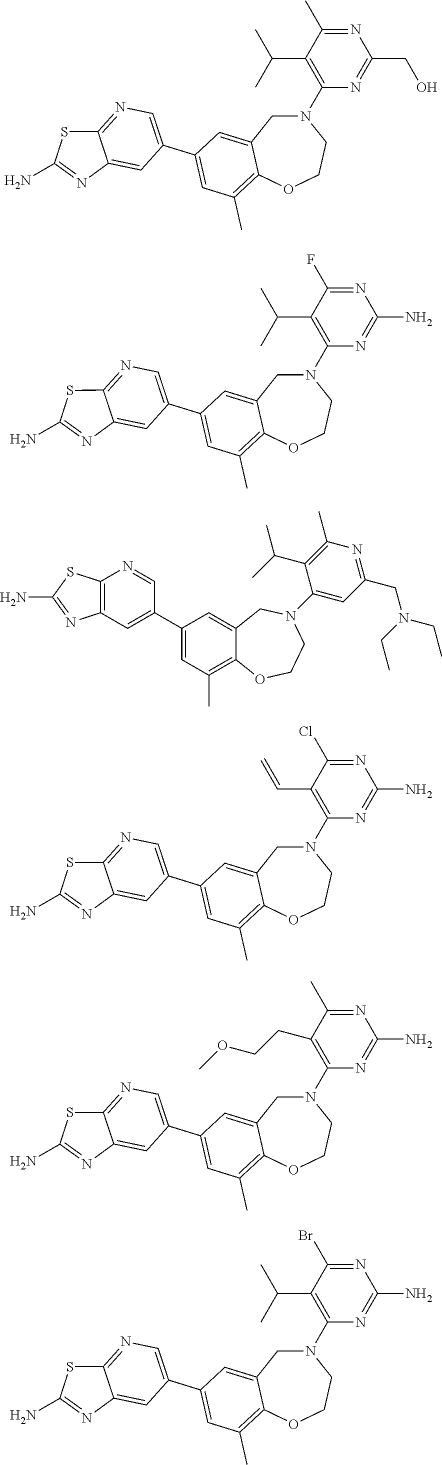

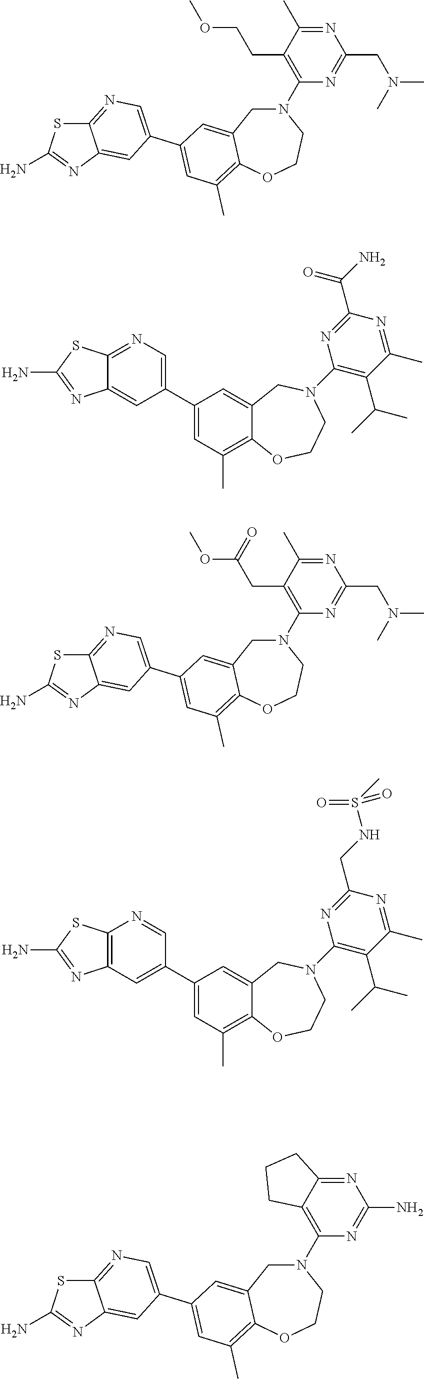 [SMILES: COCCc1c(C)nc(CN(C)C)nc1N1CCOc2c(C)cc(-c3cnc4sc(N)nc4c3)cc2C1 ; Cc1cc(-c2cnc3sc(N)nc3c2)cc2c1OCCN(c1nc(C(N)=O)nc(C)c1C(C)C)C2 ; COC(=O)Cc1c(C)nc(CN(C)C)nc1N1CCOc2c(C)cc(-c3cnc4sc(N)nc4c3)cc2C1 ; Cc1cc(-c2cnc3sc(N)nc3c2)cc2c1OCCN(c1nc(CNS(C)(=O)=O)nc(C)c1C(C)C)C2 ; Cc1cc(-c2cnc3sc(N)nc3c2)cc2c1OCCN(c1nc(N)nc3c1CCC3)C2]

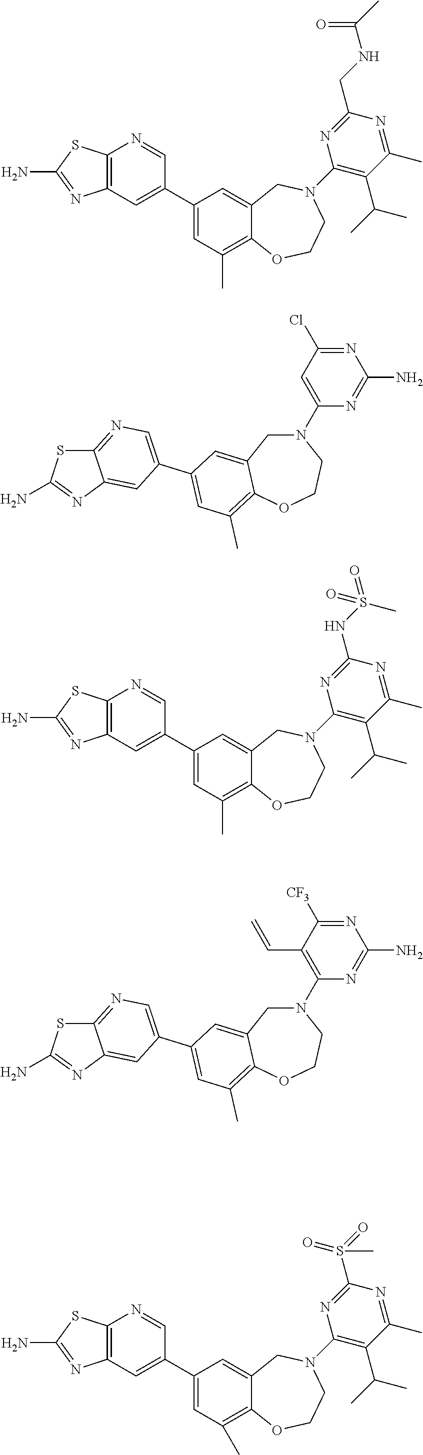 [SMILES: CC(=O)NCc1nc(C)c(C(C)C)c(N2CCOc3c(C)cc(-c4cnc5sc(N)nc5c4)cc3C2)n1 ; Cc1cc(-c2cnc3sc(N)nc3c2)cc2c1OCCN(c1cc(Cl)nc(N)n1)C2 ; Cc1cc(-c2cnc3sc(N)nc3c2)cc2c1OCCN(c1nc(NS(C)(=O)=O)nc(C)c1C(C)C)C2 ; C=Cc1c(N2CCOc3c(C)cc(-c4cnc5sc(N)nc5c4)cc3C2)nc(N)nc1C(F)(F)F ; Cc1cc(-c2cnc3sc(N)nc3c2)cc2c1OCCN(c1nc(S(C)(=O)=O)nc(C)c1C(C)C)C2]

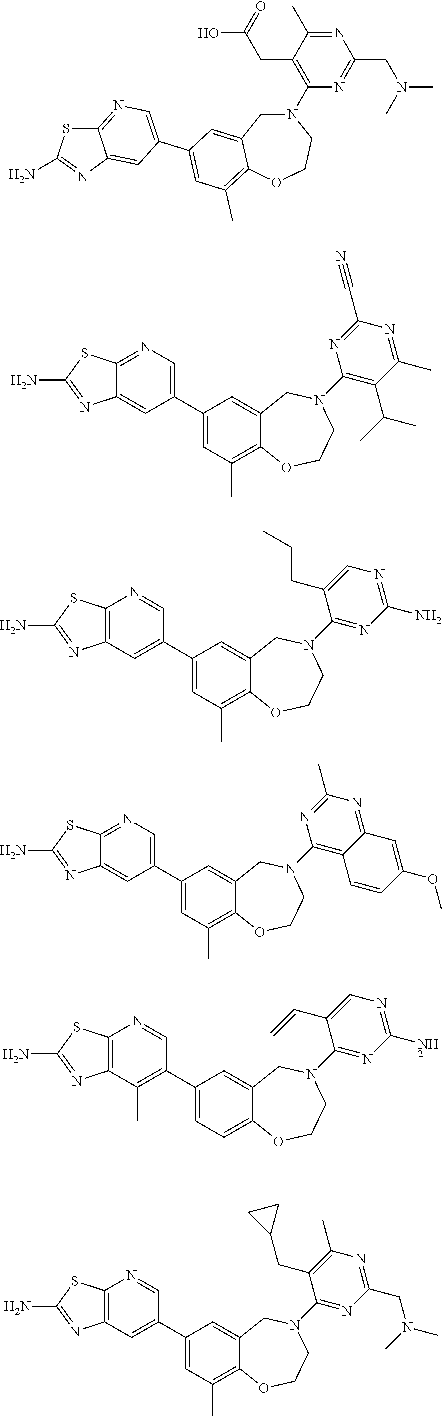 [SMILES: Cc1cc(-c2cnc3sc(N)nc3c2)cc2c1OCCN(c1nc(CN(C)C)nc(C)c1CC(=O)O)C2 ; Cc1cc(-c2cnc3sc(N)nc3c2)cc2c1OCCN(c1nc(C#N)nc(C)c1C(C)C)C2 ; CCCc1cnc(N)nc1N1CCOc2c(C)cc(-c3cnc4sc(N)nc4c3)cc2C1 ; COc1ccc2c(N3CCOc4c(C)cc(-c5cnc6sc(N)nc6c5)cc4C3)nc(C)nc2c1 ; C=Cc1cnc(N)nc1N1CCOc2ccc(-c3cnc4sc(N)nc4c3C)cc2C1 ; Cc1cc(-c2cnc3sc(N)nc3c2)cc2c1OCCN(c1nc(CN(C)C)nc(C)c1CC1CC1)C2]

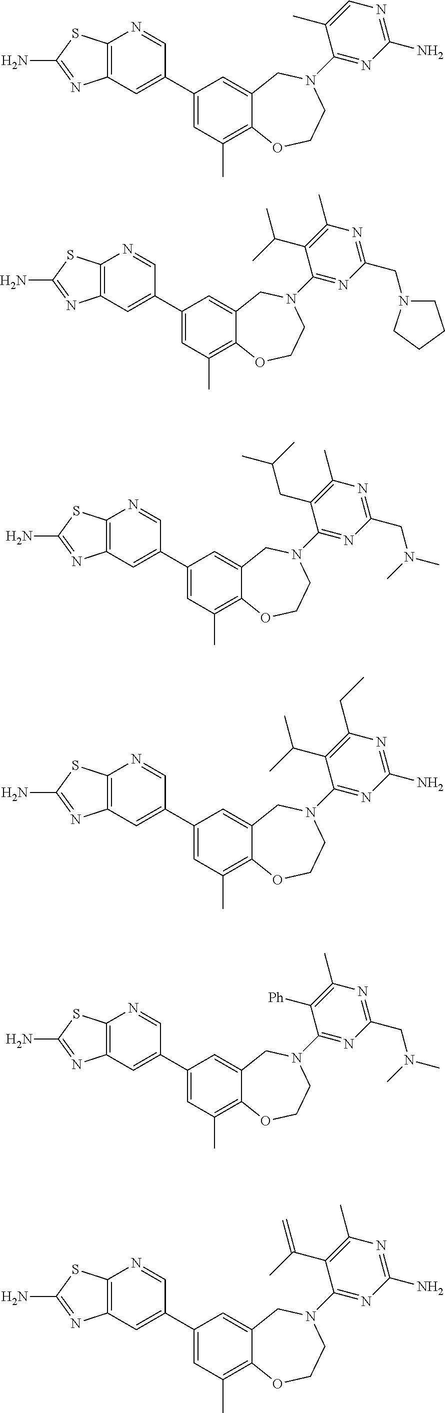 [SMILES: Cc1cnc(N)nc1N1CCOc2c(C)cc(-c3cnc4sc(N)nc4c3)cc2C1 ; Cc1cc(-c2cnc3sc(N)nc3c2)cc2c1OCCN(c1nc(CN3CCCC3)nc(C)c1C(C)C)C2 ; Cc1cc(-c2cnc3sc(N)nc3c2)cc2c1OCCN(c1nc(CN(C)C)nc(C)c1CC(C)C)C2 ; CCc1nc(N)nc(N2CCOc3c(C)cc(-c4cnc5sc(N)nc5c4)cc3C2)c1C(C)C ; Cc1cc(-c2cnc3sc(N)nc3c2)cc2c1OCCN(c1nc(CN(C)C)nc(C)c1-c1ccccc1)C2 ; C=C(C)c1c(C)nc(N)nc1N1CCOc2c(C)cc(-c3cnc4sc(N)nc4c3)cc2C1]

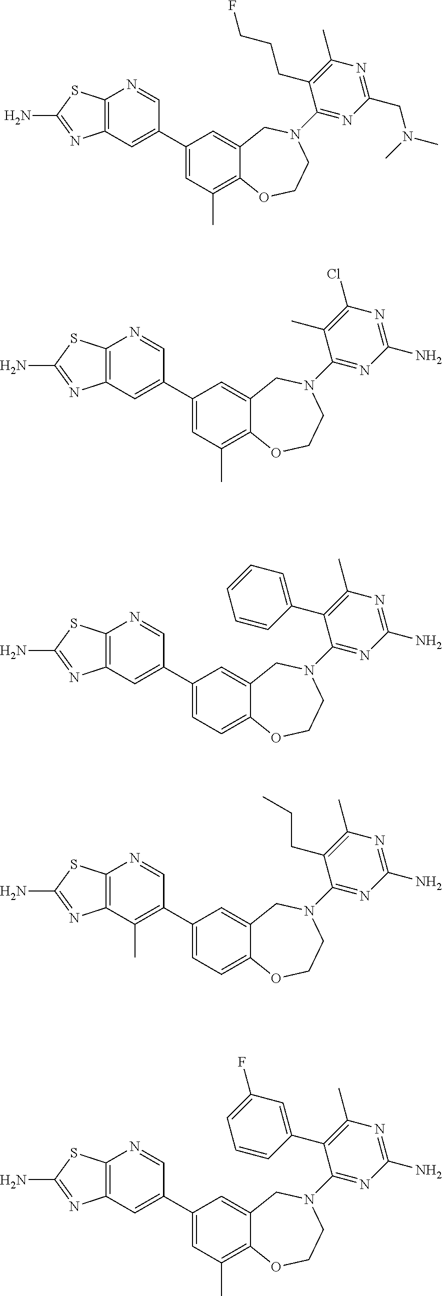 [SMILES: Cc1cc(-c2cnc3sc(N)nc3c2)cc2c1OCCN(c1nc(CN(C)C)nc(C)c1CCCF)C2 ; Cc1cc(-c2cnc3sc(N)nc3c2)cc2c1OCCN(c1nc(N)nc(Cl)c1C)C2 ; Cc1nc(N)nc(N2CCOc3ccc(-c4cnc5sc(N)nc5c4)cc3C2)c1-c1ccccc1 ; CCCc1c(C)nc(N)nc1N1CCOc2ccc(-c3cnc4sc(N)nc4c3C)cc2C1 ; Cc1cc(-c2cnc3sc(N)nc3c2)cc2c1OCCN(c1nc(N)nc(C)c1-c1cccc(F)c1)C2]

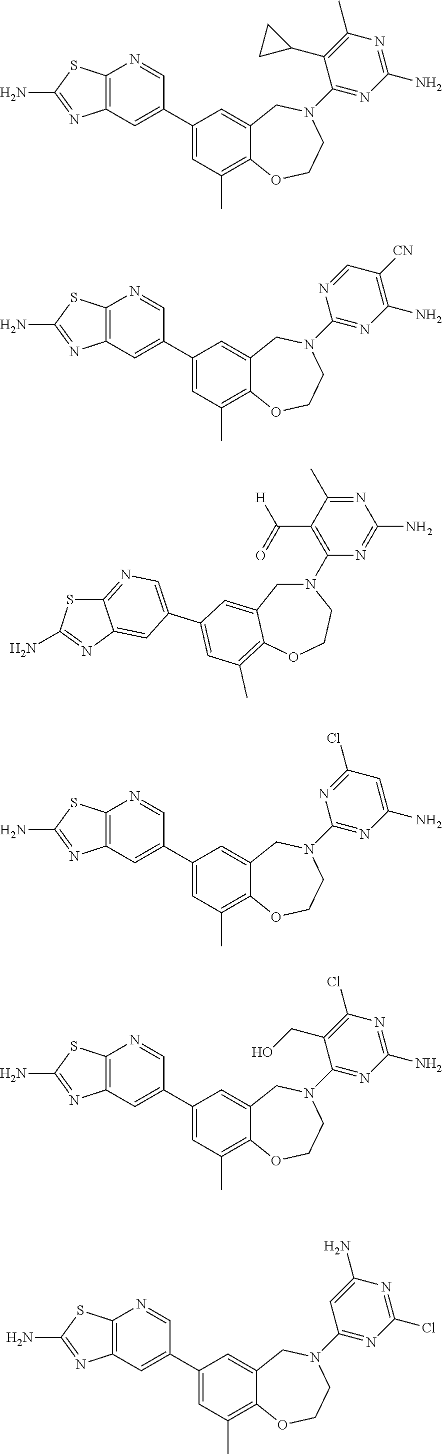

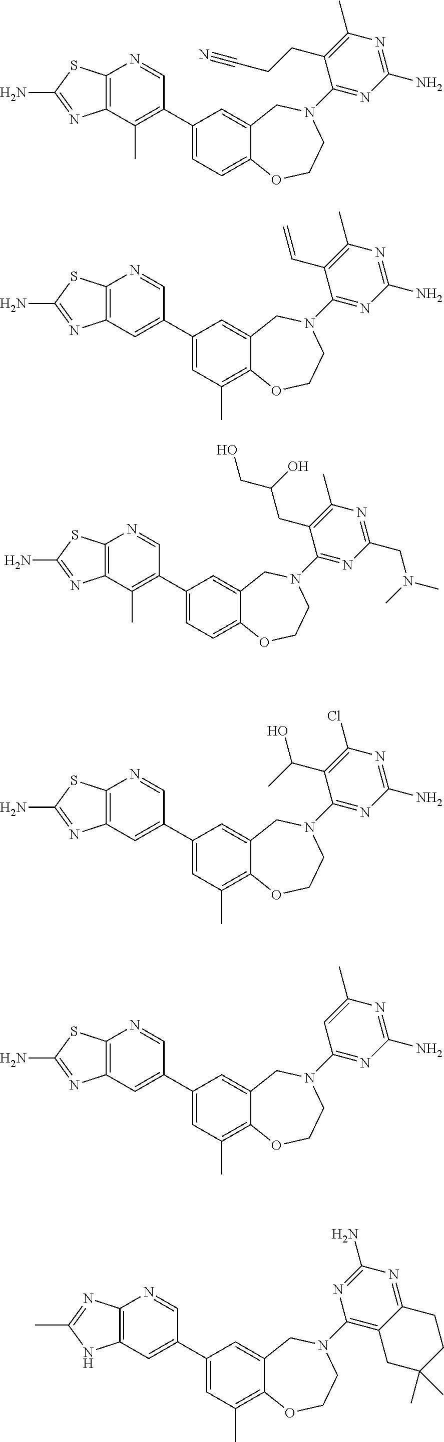

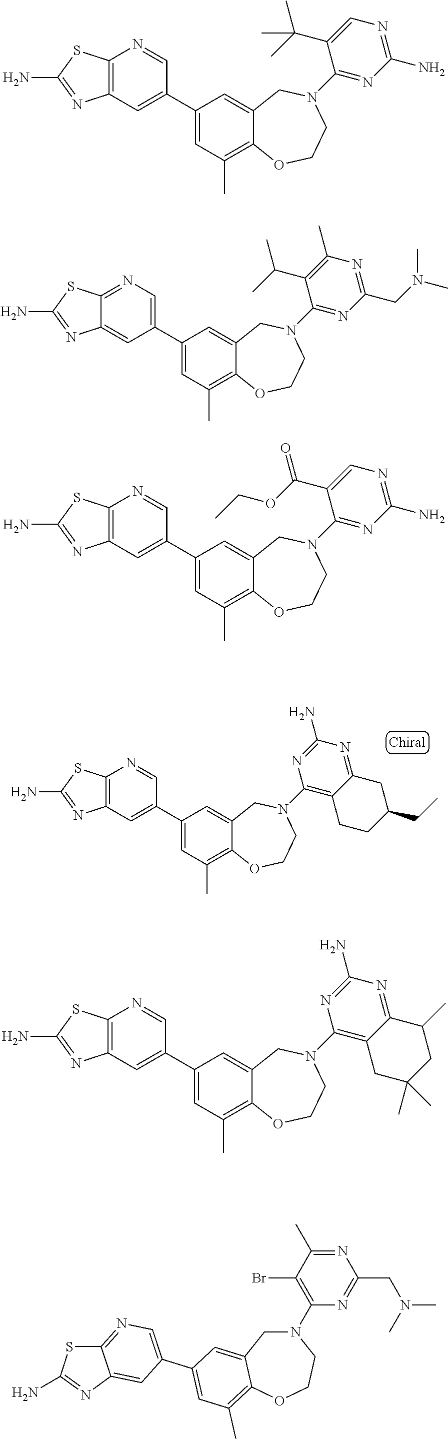 [SMILES: Cc1cc(-c2cnc3sc(N)nc3c2)cc2c1OCCN(c1nc(N)ncc1C(C)(C)C)C2 ; Cc1cc(-c2cnc3sc(N)nc3c2)cc2c1OCCN(c1nc(CN(C)C)nc(C)c1C(C)C)C2 ; CCOC(=O)c1cnc(N)nc1N1CCOc2c(C)cc(-c3cnc4sc(N)nc4c3)cc2C1 ; CC[C@H]1CCc2c(nc(N)nc2N2CCOc3c(C)cc(-c4cnc5sc(N)nc5c4)cc3C2)C1 ; Cc1cc(-c2cnc3sc(N)nc3c2)cc2c1OCCN(c1nc(N)nc3c1CC(C)(C)CC3C)C2 ; Cc1cc(-c2cnc3sc(N)nc3c2)cc2c1OCCN(c1nc(CN(C)C)nc(C)c1Br)C2]

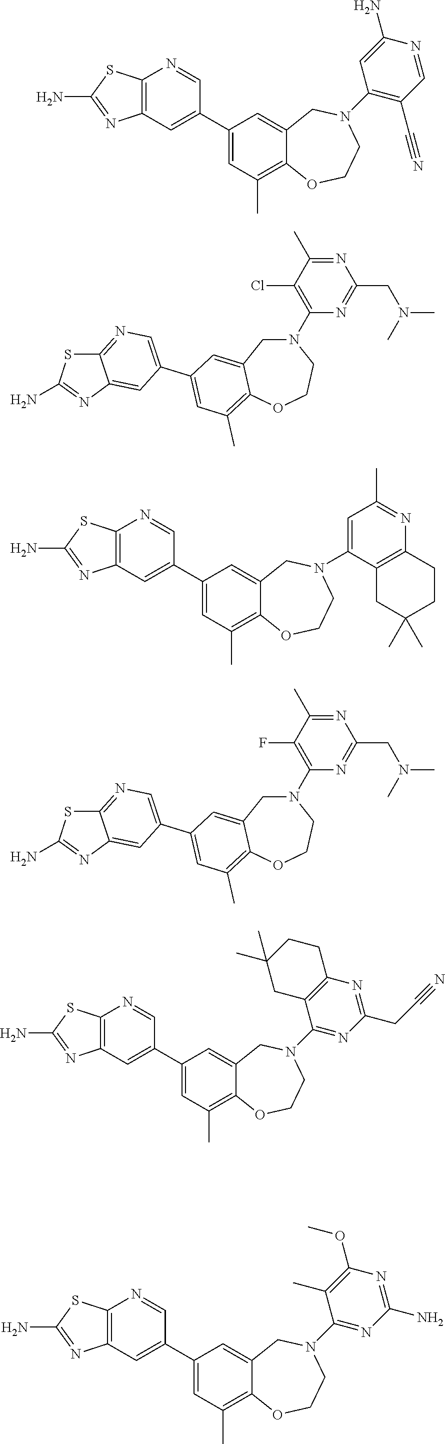

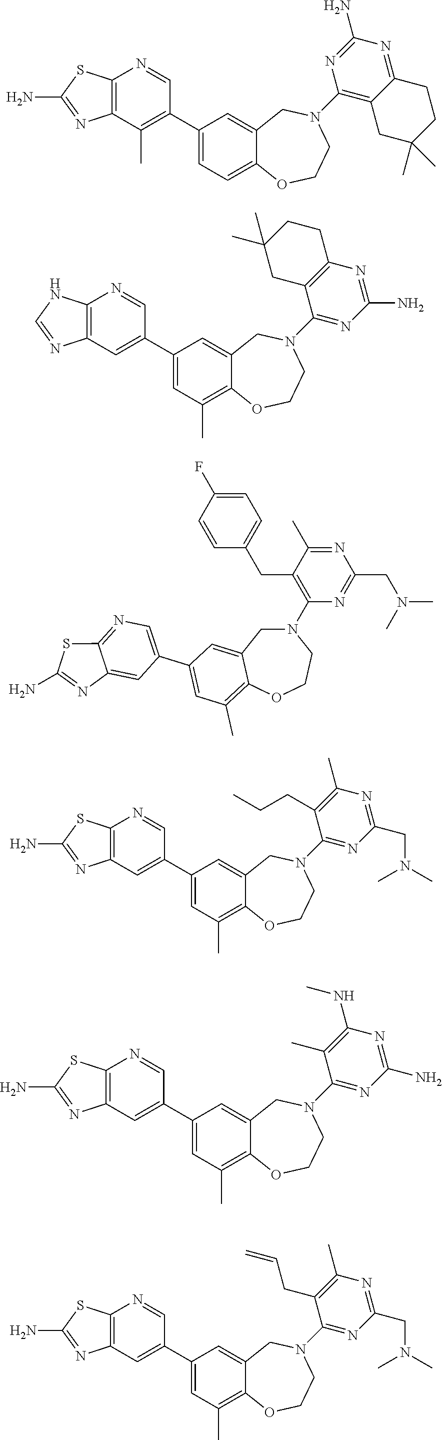

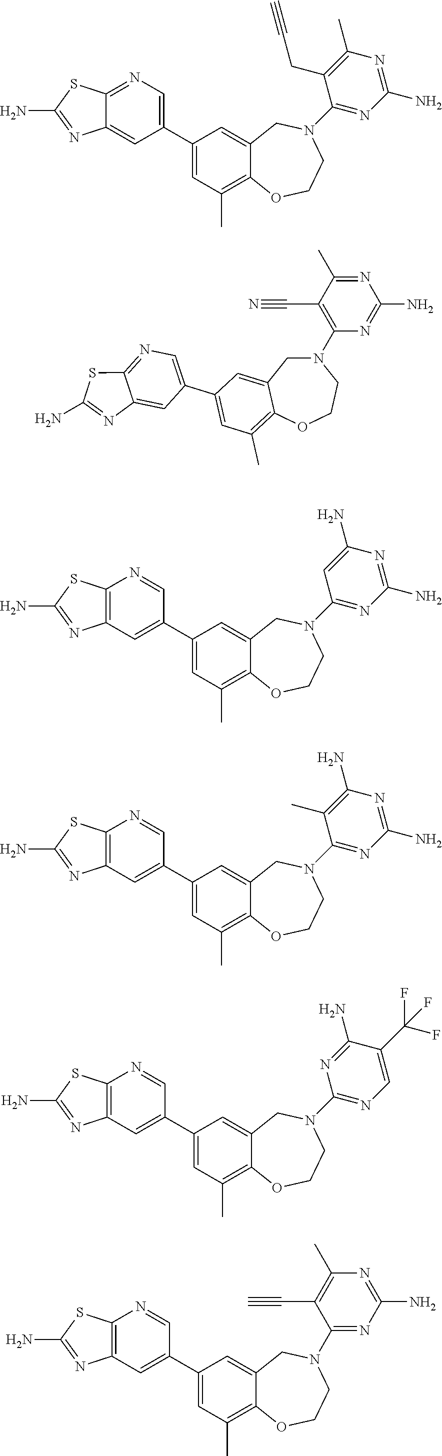 [SMILES: C#CCc1c(C)nc(N)nc1N1CCOc2c(C)cc(-c3cnc4sc(N)nc4c3)cc2C1 ; Cc1cc(-c2cnc3sc(N)nc3c2)cc2c1OCCN(c1nc(N)nc(C)c1C#N)C2 ; Cc1cc(-c2cnc3sc(N)nc3c2)cc2c1OCCN(c1cc(N)nc(N)n1)C2 ; Cc1cc(-c2cnc3sc(N)nc3c2)cc2c1OCCN(c1nc(N)nc(N)c1C)C2 ; Cc1cc(-c2cnc3sc(N)nc3c2)cc2c1OCCN(c1ncc(C(F)(F)F)c(N)n1)C2 ; C#Cc1c(C)nc(N)nc1N1CCOc2c(C)cc(-c3cnc4sc(N)nc4c3)cc2C1]

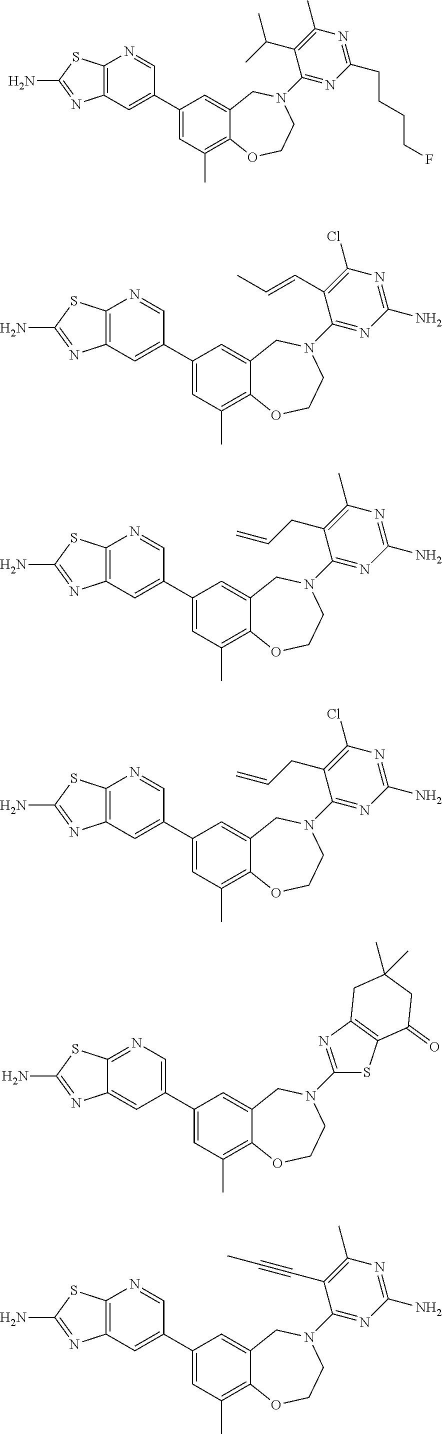 [SMILES: Cc1cc(-c2cnc3sc(N)nc3c2)cc2c1OCCN(c1nc(CCCCF)nc(C)c1C(C)C)C2 ; C/C=C/c1c(Cl)nc(N)nc1N1CCOc2c(C)cc(-c3cnc4sc(N)nc4c3)cc2C1 ; C=CCc1c(C)nc(N)nc1N1CCOc2c(C)cc(-c3cnc4sc(N)nc4c3)cc2C1 ; C=CCc1c(Cl)nc(N)nc1N1CCOc2c(C)cc(-c3cnc4sc(N)nc4c3)cc2C1 ; Cc1cc(-c2cnc3sc(N)nc3c2)cc2c1OCCN(c1nc3c(s1)C(=O)CC(C)(C)C3)C2 ; CC#Cc1c(C)nc(N)nc1N1CCOc2c(C)cc(-c3cnc4sc(N)nc4c3)cc2C1]

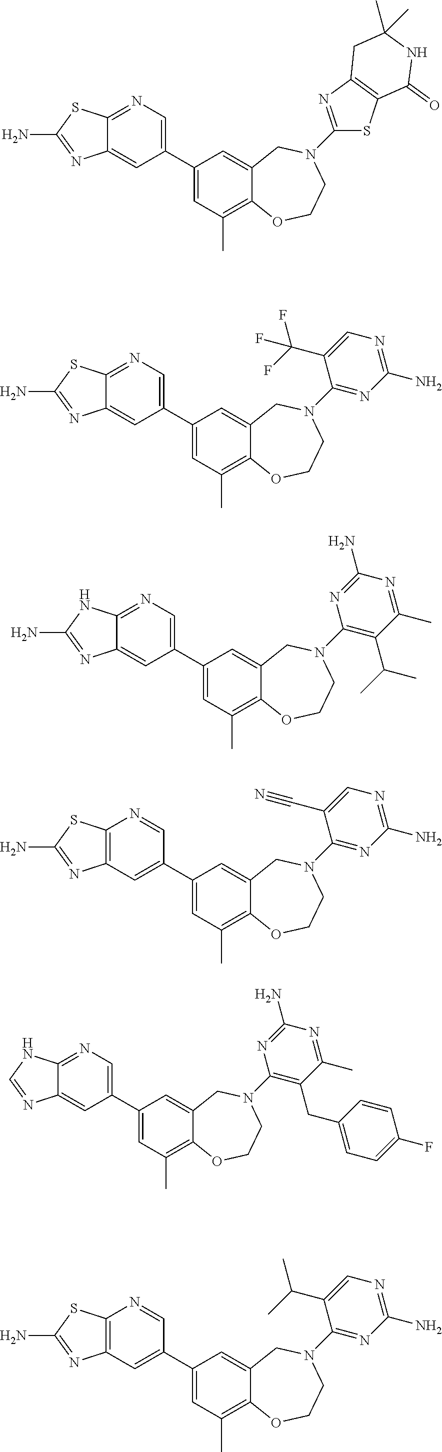 [SMILES: Cc1cc(-c2cnc3sc(N)nc3c2)cc2c1OCCN(c1nc3c(s1)C(=O)NC(C)(C)C3)C2 ; Cc1cc(-c2cnc3sc(N)nc3c2)cc2c1OCCN(c1nc(N)ncc1C(F)(F)F)C2 ; Cc1cc(-c2cnc3[nH]c(N)nc3c2)cc2c1OCCN(c1nc(N)nc(C)c1C(C)C)C2 ; Cc1cc(-c2cnc3sc(N)nc3c2)cc2c1OCCN(c1nc(N)ncc1C#N)C2 ; Cc1cc(-c2cnc3[nH]cnc3c2)cc2c1OCCN(c1nc(N)nc(C)c1Cc1ccc(F)cc1)C2 ; Cc1cc(-c2cnc3sc(N)nc3c2)cc2c1OCCN(c1nc(N)ncc1C(C)C)C2]

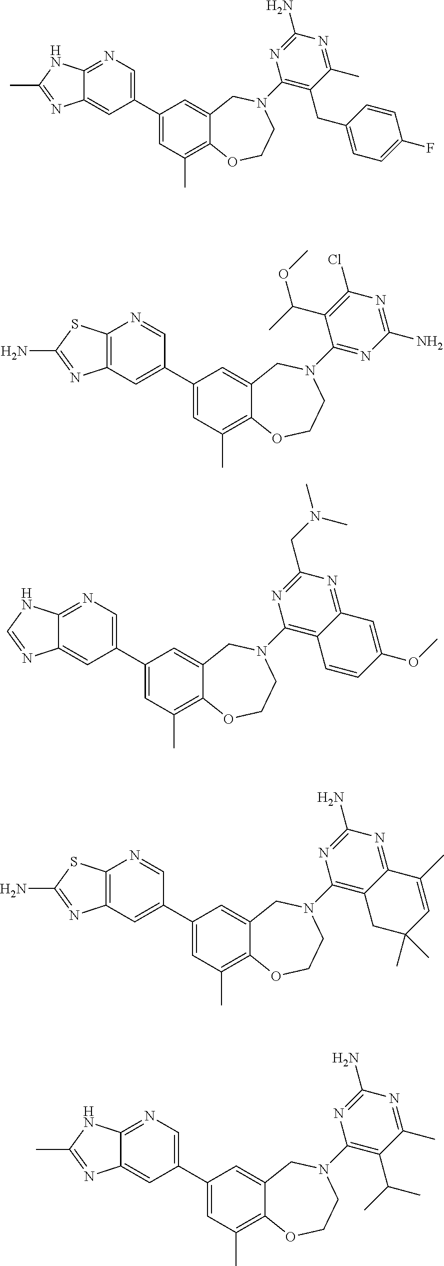

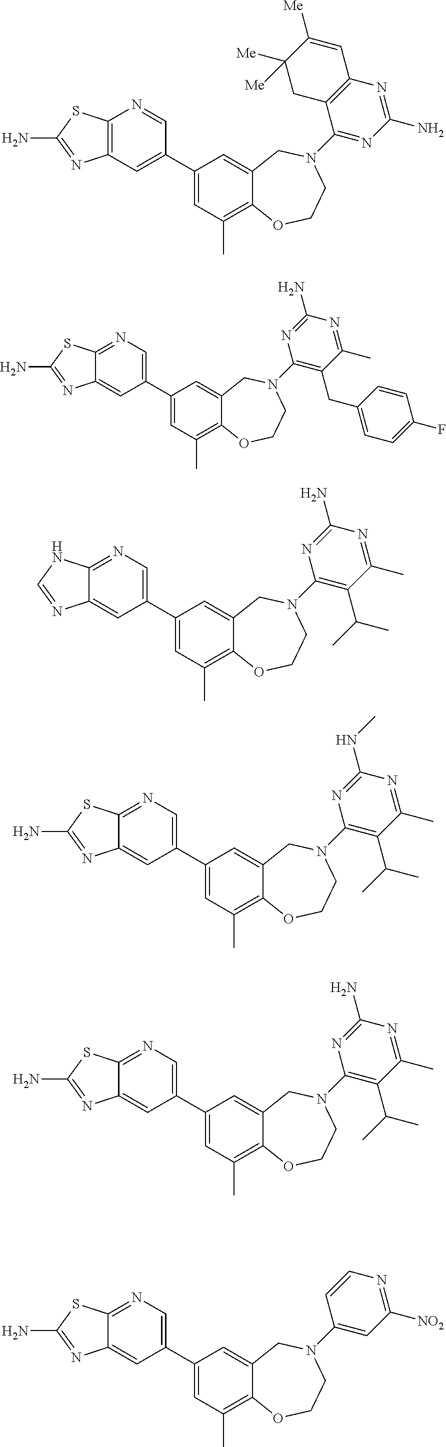 [SMILES: CC1=Cc2nc(N)nc(N3CCOc4c(C)cc(-c5cnc6sc(N)nc6c5)cc4C3)c2CC1(C)C ; Cc1cc(-c2cnc3sc(N)nc3c2)cc2c1OCCN(c1nc(N)nc(C)c1Cc1ccc(F)cc1)C2 ; Cc1cc(-c2cnc3[nH]cnc3c2)cc2c1OCCN(c1nc(N)nc(C)c1C(C)C)C2 ; CNc1nc(C)c(C(C)C)c(N2CCOc3c(C)cc(-c4cnc5sc(N)nc5c4)cc3C2)n1 ; Cc1cc(-c2cnc3sc(N)nc3c2)cc2c1OCCN(c1nc(N)nc(C)c1C(C)C)C2 ; Cc1cc(-c2cnc3sc(N)nc3c2)cc2c1OCCN(c1ccnc([N+](=O)[O-])c1)C2]

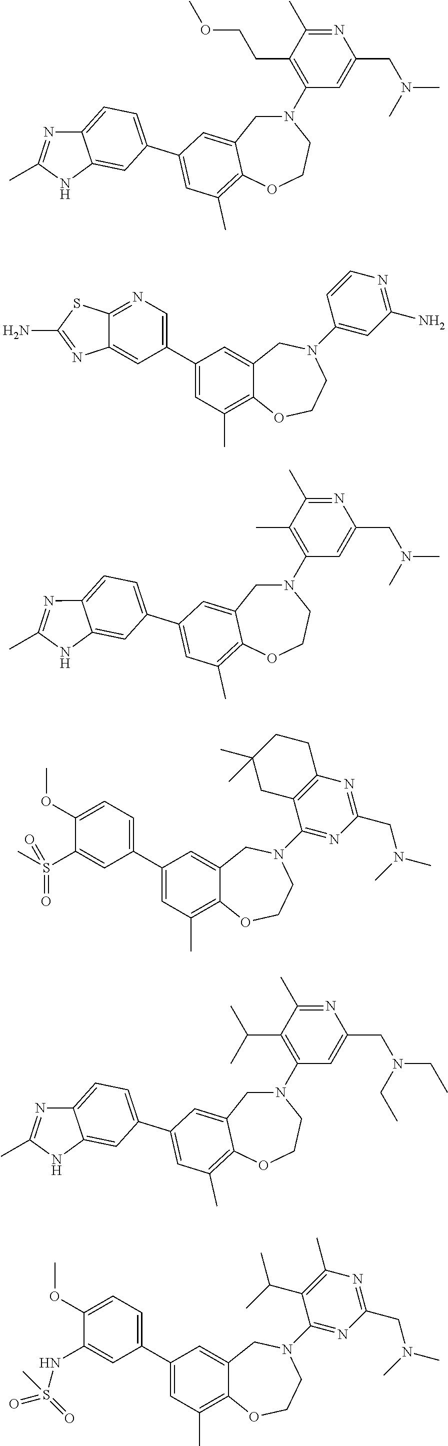

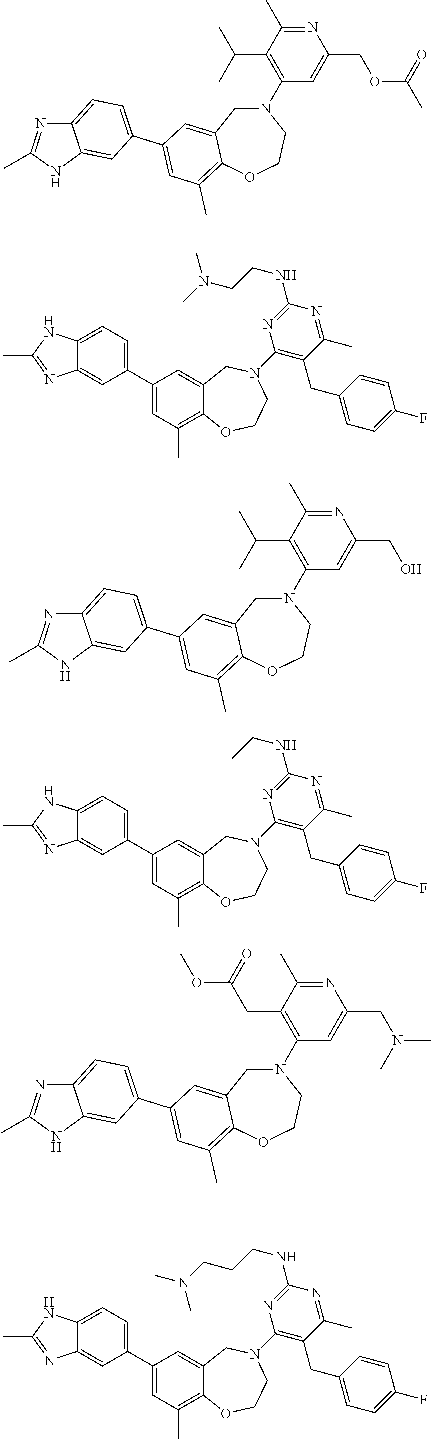

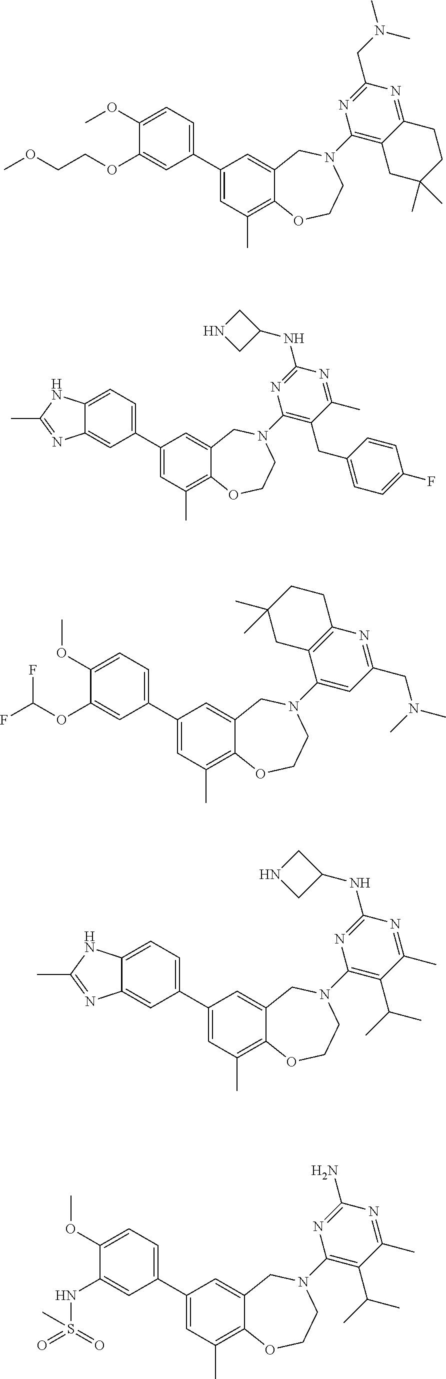

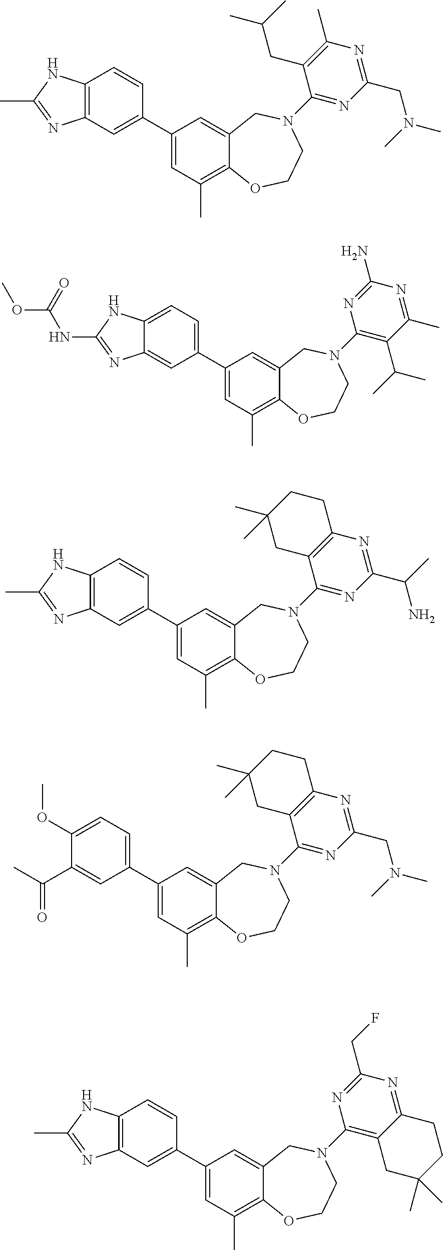

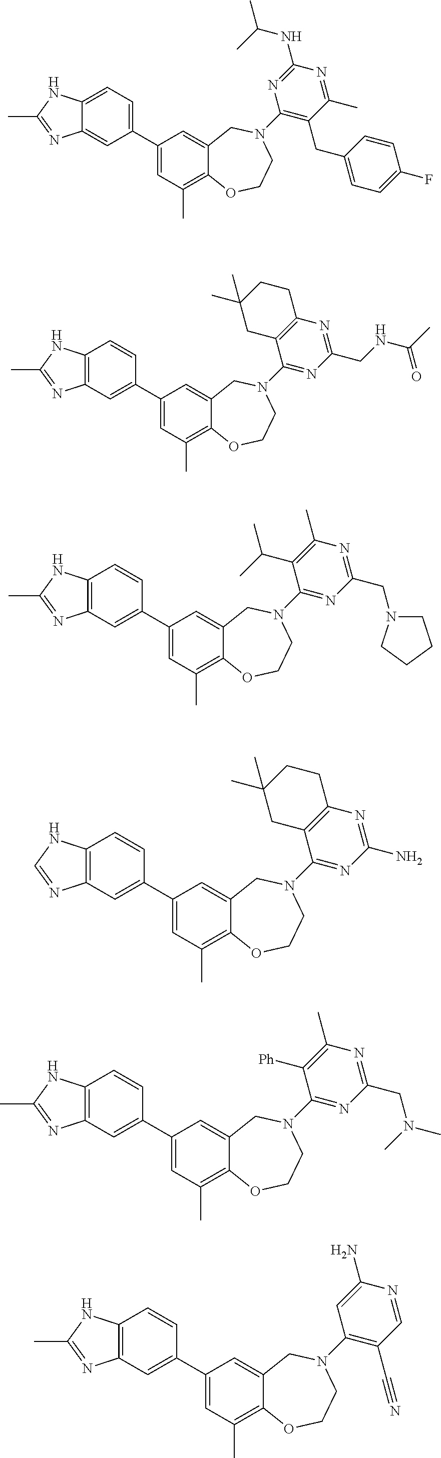

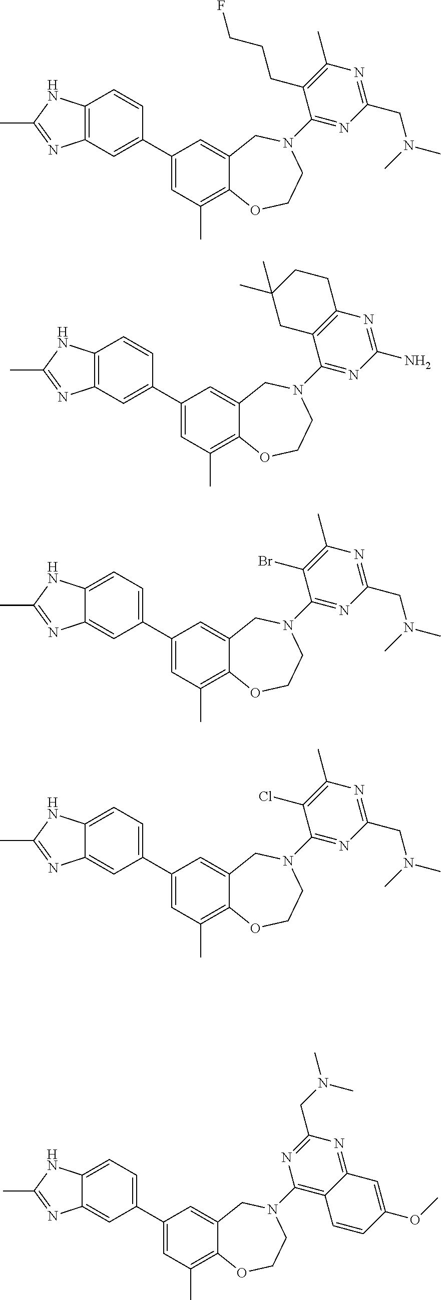

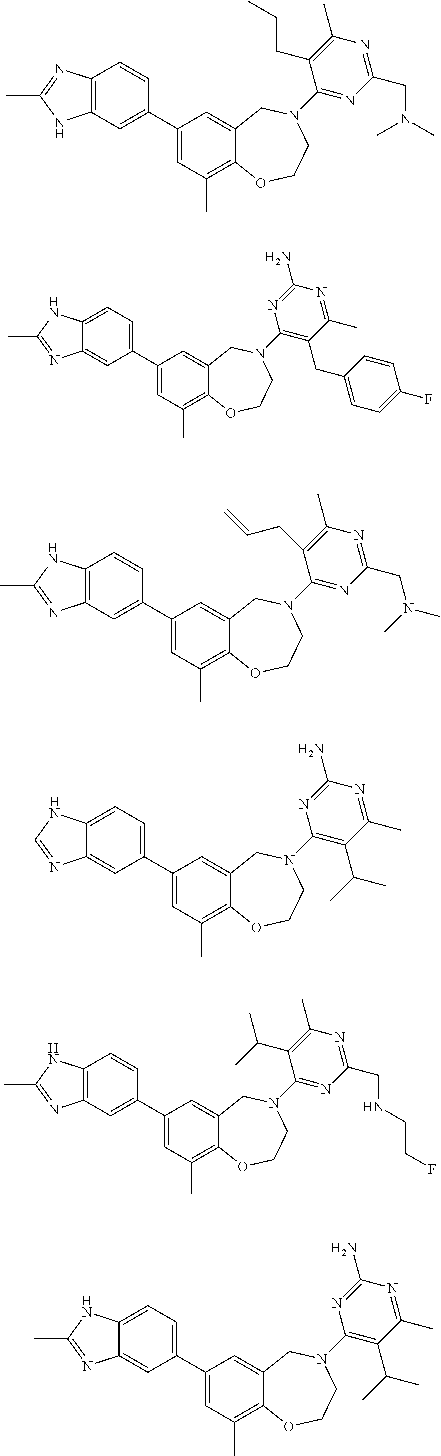

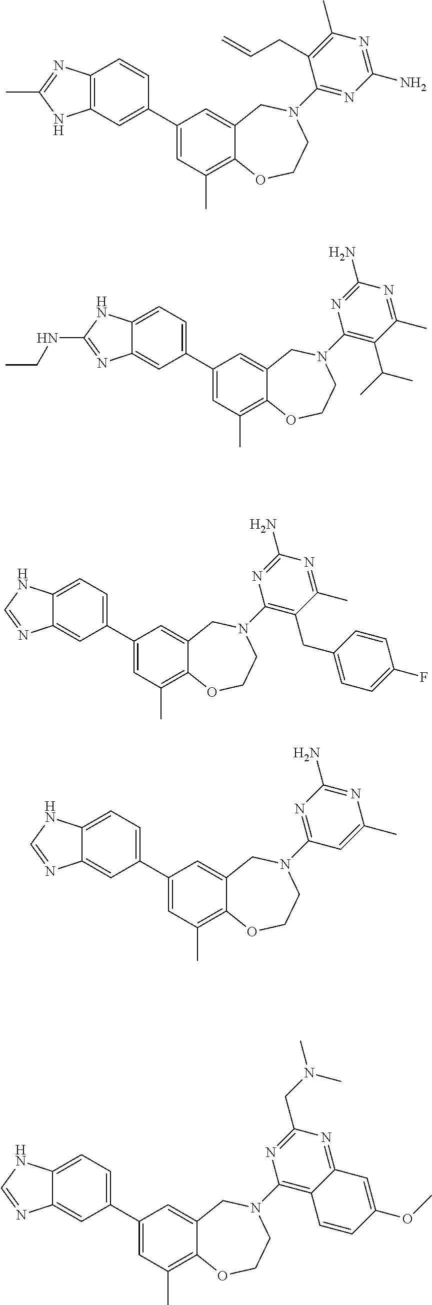 [SMILES: C=CCc1c(C)nc(N)nc1N1CCOc2c(C)cc(-c3ccc4nc(C)[nH]c4c3)cc2C1 ; CCNc1nc2cc(-c3cc(C)c4c(c3)CN(c3nc(N)nc(C)c3C(C)C)CCO4)ccc2[nH]1 ; Cc1cc(-c2ccc3[nH]cnc3c2)cc2c1OCCN(c1nc(N)nc(C)c1Cc1ccc(F)cc1)C2 ; Cc1cc(N2CCOc3c(C)cc(-c4ccc5[nH]cnc5c4)cc3C2)nc(N)n1 ; COc1ccc2c(N3CCOc4c(C)cc(-c5ccc6[nH]cnc6c5)cc4C3)nc(CN(C)C)nc2c1]

Useful Intermediates: 4-[6,7-bis(methyloxy)quinolin-4-yl]7-bromo-2,3,4,5- tetrahydro-1,4-benzoxazepine; 4-{4-[6,7-bis(methyloxy)quinolin-4-yl]-2,3,4,5-tetrahydro- 1,4-benzoxazepin-7-yl}-2-nitroaniline; 4-{4-[6,7-bis(methyloxy)quinolin-4-yl[-2,3,4,5- tetrahydro-1,4-benzoxazepin-7-yl}benzene-1,2-diamine: N-[5-(4-{5-[(4- fluorophenyl)methyl]-6-methylpyrimidine-4-yl-56 -2,3,4,5-tetrahydro-1,4-benzoxazepin-7-yl)- 1,3-thiazol-2-yl]acetamide; 7-bromo-4-{5-[(4-fluorophenyl)methyl]-6-methylpyrimidin-4- yl}-2,3,4,5-tetrahydro-1,4-benzoxazepine; 4-[6,7-bis(methyloxy)quinazolin-4-yl]-7-bromo- 2,3,4,5-tetrahydro-1,4-benzoxazepine; 7-bromo-4-[6-(methyloxy)quinazolin-4-yl]-2,3,4,5- tetrahydro-1,4-benzoxazepine.

### General Administration

In one aspect, the invention provides pharmaceutical compositions comprising an inhibitor of PI3K and/or mTOR according to the invention and a pharmaceutically acceptable, carrier, excipient, or diluent. In certain other specific embodiments, administration is by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, of rectally, in the form of solid, semi-solid, lyophilized powder, or liquid dosage forms, such as for example, tablets, suppositories, pills, soft clastic and hard gelatin capsules powders, solutions, suspensions, or aerosols, or the like, specifically in unit dosage forms suitable for simple administration of precise dosages.

The compositions will include a conventional pharmaceutical carrier or excipient and a Compound of the invention as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary, substances such as welting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example. U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that

Compositions suitable for parenteral injection may comprise physiologically acceptable sterile aqueous or nonaqueous solutions, dispersions, suspensions or emulsions, and sterile powders for reconstilution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active Compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders as for example paraffin, (f) absorption accelerators, as for example, quaternary ammonium compounds, (g) welling agents, as for example, cetyl alcohol, and glycerol monostearate, magnesium stearale and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active Compound or compounds in a certain part Of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoale, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbiian; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butler, polyethyleneglycol or a suppository wax, which are solid al ordinary temperatures but liquid at body temperature and therefore, melt while in a suitable body cavity and release the active component therein.

Dosage forms for topical administration of a Compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a Compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a conipound(s) of the invention, or a pharmaceutically acceptable salt thereof, with the rest being suitable pharmaceutical excipients.

Actual methods of preparing such dosage forms are known, or will be apparent, to those skilled in this art: for example, see Remington's Pharmaceutical Sciences, 18th Ed., (Mack Publishing Company, Easton. Pa., 1990). The composition to be administered will, in any event, contains therapeutically effective amount of a Compound of the invention, or a pharmaceutically acceptable salt thereof, for treatment of a disease-state in accordance with the teachings of this invention.

The compounds of the invention, or their pharmaceutically acceptable salts or solvates, are administered in a therapeutically effective amount which will vary depending upon a variety of factors including the activity of the specific Compound employed, the metabolic stability and length of action of the compound, the age, body weight, general health, sex, diet, mode and time of administration, rate of excretion, drug combination, the severity of the particular disease-states, and the host undergoing therapy. The compounds of the present invenlion can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and ihe pharmacological activity of the Compound being used. The determination of optimum dosages for a particular patient is well known to one of ordinary skill in the art.

If formulated as a fixed dose, such combination products employ the compounds of this invention within ihe dosage range described above and ihe other pharmaceutically active agent(s) within its approved dosage range. Compounds of the instant invention may alternatively be used sequentially with known pharmaceutically acceptable agent(s) when a combination formulation is inappropriate.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif), or are prepared by methods known to those skilled in the art following procedures set forth in references such as Ficser and Fieser's Reagents for Organic Synthesis, Volumes 1-17 (John Wiley and Sons, 1991); Rodd's Chemistry of Carbon Compounds, Volumes 1-5 and Supplemental (Elsevier Science Publishers, 1989): Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials niay be characterized using conventional means, including physical constants and spectral data.

Unless specified to the contrary, the reactions described herein take place at atmospheric pressure and over a temperature range from about −78° C. to about 150° C., more, specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These, techniques generally modify appropriate functional groups in a given compound. These modified functional groups regenerate original functional groups by routine manipulation or in vivo. Amides and esters of the compounds of the present invention may be prepared according lo conventional methods. A thorough discussion of prodrugs is provided in T. Higuchi and V. Stella, “Pro-drugs as Novel Delivery Systems,” Vol 14 of ihe A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of the Invention may exist as single stereoisomers, racemates, and as mixtures of enantiomers and diastereomers. The compounds, may also exist as geometric isomers. All such single stereoisomers racemates and mixtures thereof, and geometric isomers are intended to be within the scope of this invention.

Some of the compounds of the invention contain an active ketone —C(O)CF₃ and may exist in part or in whole as the —C(OH₂)CF₃ form. Regardless of whether the Compound is. drawn as the —C(O)CF₃ , or —C(OH₂)CF₃ form, both are included within the scope of the Invention. Although an individual Compound may be drawn as the —C(O)CF₃ form, one of ordinary skill in the art would understand that the Compound may exist in part or in whole as the —C(OH₂)CF₃ form and that the ratio of the two forms may vary depending on the Compound and the conditions in which it exists.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention. Further, for example, in this application R¹ can be 5-oxo-1H-1,2,4-triazol-3-yl, depicted structurally as

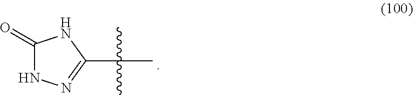 [SMILES: *c1n[nH]c(=O)[nH]1]

Both 5-oxo-1H-1,2,4-triazol-3-yl and the structure 100 include, and are equivalent to, 3-hydroxy-4H-1,2,4-triazolo-yl and its structure

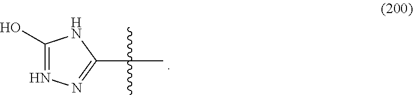 [SMILES: *c1nnc(O)[nH]1]

In another example, in this application R¹ can be 2-imino-1(2H)- hydroxy-pyrimidin-5-yl, depicted structurally as

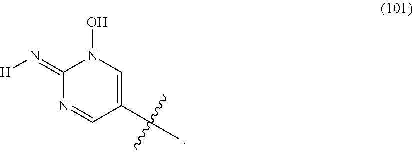 [SMILES: *c1cn/c(=N\[H])n(O)c1]

Both 2-imino- 1(2H)- hydroxy-pyrimidin-5-yl and the structure 101 include, and are equivalent to, N-oxide of 2- amino-pyrimidin-5-yl and its structure 201:

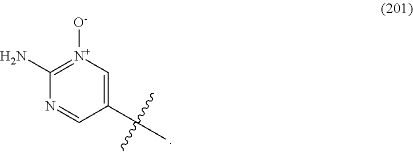 [SMILES: *c1cnc(N)[n+]([O-])c1]

Regardless of which structure or which terminology is used, each tautomer is included within the scope of the Invention.

The present invention also includes N-oxide derivatives and protected derivatives of compounds of the Invention. For example, when compounds of the Invention contain an oxidizable-nitrogen atom, the nitrogen atom can be converted to an N-oxide by methods well knownin the art. When compounds of the Invention contain groups such as hydroxy, carboxy, thiol or any group containing a nitrogen atom(s), these groups can be protected with a suitable “protecting group” or “protective group”. A comprehensive list of suitable protective groups can be found in T.W. Greene, Protective Groups in Organic Synthesis, John Wiley & Sons, Inc. 1991, the disclosure of which is incorporated herein by reference in its entirely. The protected derivatives of compounds of the Invention can be prepared by methods well known in the art.

Methods for the preparation and/or separation and isolation of single stereoisomers from racemic mixtures or non-racemic mixtures of stereoisomers are well known in the art. For example, optically active (R)- and (S)- isomers may be prepared using chiral synthons or chiral reagents, or resolved using conventional techniques. Enantiomers (R- and S-isomers) may be resolved by methods known to one of ordinary skill in the art, for example by: formation of diastereosiomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted, into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvatcd as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. The following examples illustrate but do not limit the invention. All references cited herein are incorporated by reference in their entirety.

An intermediate of formula 4 where PG is a nitrogen-protecting group, R^{5a} and R^{5c} are independently hydrogen or alkyl, R^{5h} is hydrogen or halo. R^{5b} is (C₁₋₃) alkyl, and R^{5d}, R^{5c}, R^{5f}, and R^{5g} are hydrogen can be prepared according to Scheme 1.

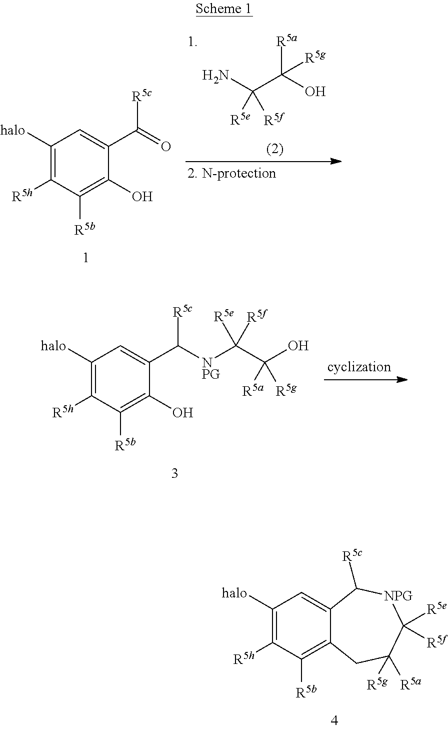

In particular, an intermediate of formula 4a can be prepared according to Scheme 1a.

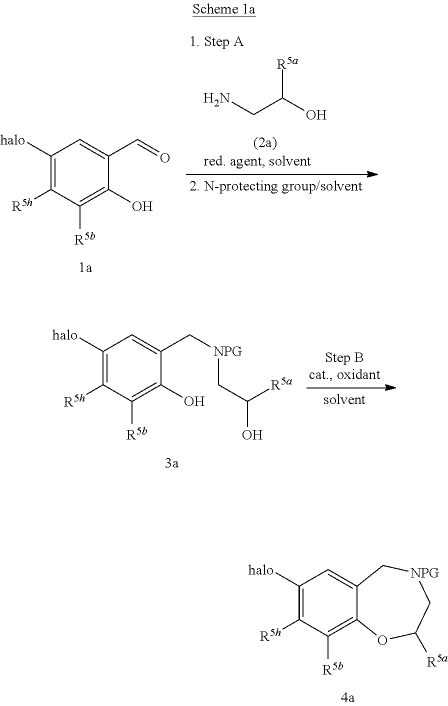

An intermediate of formula 2a where R^{5a}, is hydrogen or methyl is commercially available. The intermediate of formula 1a is treated with an intermediate of formula 2a in the presence of a reducing agent such as sodium borohydride, in a solvent(s) such as tetrahydrofuran and/or methanol and allowed to react at a temperature of about 40° C. for approximately 4 hours. The solvent is then removed and the reaction is taken up in a solvcnt(s) such as ethyl acetate and/or saturated sodium bicarbonate. To this suspension a nitrogen-protecting group precursor, such as di-tert-butyl dicarbonate, is added and the mixture is allowed to stir at room temperature overnight to yield an intermediate of formula 3a where PC is a nitrogen-protecting group.

Intermediate 3a is then ireated with a catalyst, such as triphenylphosphine, in the presence of a dehydrating agent such as diisopropyl azodicarboxylate, in a solvent such as DCM. The reaction is allowed to proceed at room temperature for approximately 12 hours and the resulting product is optionally purified by column chromatography to yield an intermediate of formula 4a. Alternatively, the intermediate of formula 4a can be prepared by treating the intermediate of formula 3a with Burgess' reagent.

An intermediate of formula 5 where each R is hydrogen or both R's when taken together form a cyclic boronic ester. PG is a nitrogen-protecting group. R^{5a} and R^{5c} are independently hydrogen or alkyl, R^{5h} is hydrogen or halo, R^{5h} is (C₁₋₃)alkyl, R^{5c}, R^{5f} and R^{5g} are hydrogen, and R¹ is as defined in the Summary of the Invention for a Compound of Formula 1 can be prepared according to Scheme 2.

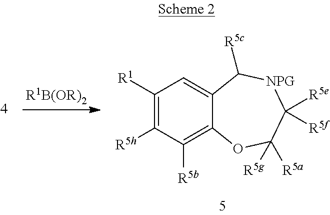

where, the intermediate of formula 4 is prepared as described in Scheme 1.

In particular, an intermediate of formula 5a where R^{5a} is hydrogen or alkyl, R^{5h} is hydrogen or halo, R^{5b} is (C₁₋₃)alkyl, and R¹ is as defined in the Summary of the Invention for a Compound of Formula I, can be prepared according to Scheme 2a.

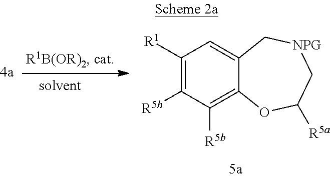

The intermediate of formtda 4a, prepared as described in Scheme 1a, is treated with a boronic acid of formula R¹B(OH)₂ or

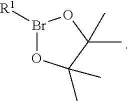

which are commercially available or can be prepared using procedures known to one of ordinary skill in the art. The reaction is carried out in the presence of a catalyst such as Pd(dppf)₂Cl₂, a base such as potassium carbonate, and in a solvent such as DME at about 80° C. for about 2 hours. The product can then be purified by chromatography to yield an intermediate of formula 5a.

Alternatively, an intermediate of formula 5, as defined above, can be prepared as described in Scheme 4.

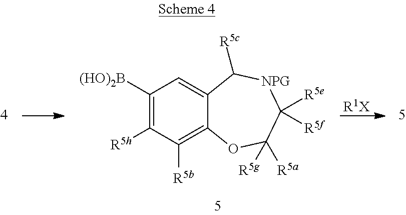

In particular, an intermediate of formula 5b where PG is a nitrogen-protecting group and R¹ and R^{5b} are as defined in the Summary of the Invention for a Compound of Formula I can be prepared according to Scheme 4a.

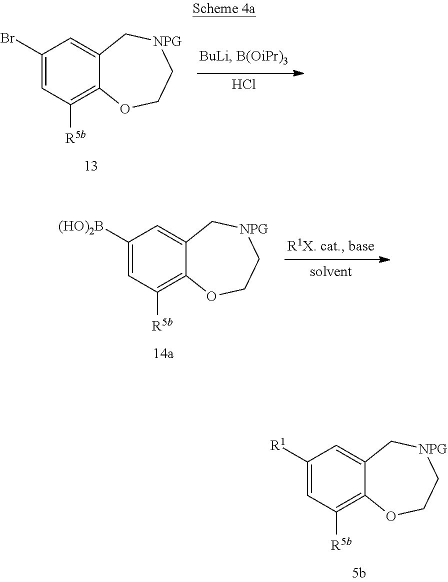

An intermediate formula 13, where PG is a nitrogen-protecting group, is prepared as described in Scheme 1a, 13 is treated with triisopropylborate in a solvent such as THF at a temperature of about −60° C., followed by dropwise addition of a base such as n-butyllithium in tetrahydrofuran. The reaction was allowed to proceed for about 30 minutes, was treated with an acid such as hydrochloric acid, and allowed to warm to room temperature to yield an intermediate of formula 14a. Intermediate 14a is then treated with an intermediate of formula R¹X (where X is a halide, and which is commercially available or can be prepared using procedures known to one of ordinary skill in the art), in the presence of a base such as potassium carbonate, in the presence of a catalyst such as

tetrakis(triphenylphosphine)palladium(0), and in a solvent(s) such as 1,2-dimethoxyethane and/or water. The reaction is allowed to proceed under nitrogen and stirred at reflux for about 3 hours io yield an intermediate of formula 5b.

In particular, a Compound of the Invention where Y is =CH- or =N-, R^{5a}, R^{5c}, R^{5d}, R^{5c}, R^{5f}, R^{5g}, and R^{5h} are hydrogen: R¹ is benzimidazol-6-yl substituted at the 2-position with one R⁷; R⁷ is alkyl; R² and R^{5h} and all other groups are independently as defined in the Summary of the Invention for a Compound of Formula 1, can be prepared according to Scheme 6a.

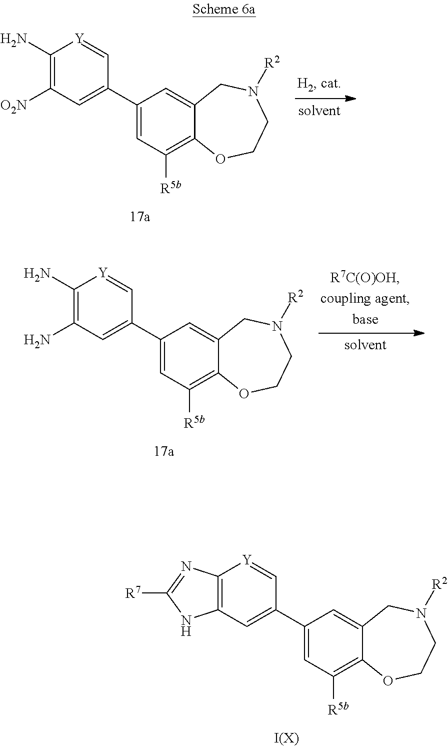

The nitro of the intermediate of formula 17a, prepared as described above in Scheme 4, is reduced in the presence of H₂ and palladium on carbon in a solvent(s) such as methanol and/or acetic acid to yield an intermediate of formula 18a. The intermediate of formula 18a is then treated with an intermediate of formula R⁷C(O)OH, in the presence of a coupling agent such as HATU. in the presence, of a base such as DIEA, in a solvent(s) such as DMF and/or acetic acid. The product can be purified by column chromatography to yield a Compound of Formula 1(x).

A Compound of the Invenlion of Formula 1 where R^{5a}, and R^{5c} are independently hydrogen or alkyl, R^{5h} is hydrogen or halo, R^{5h} is (C₁₋₃)alkyl, R^{5c}, R^{5f}, and R^{5g} are hydrogen, and R¹ and R² are independently as defined in ihe Summary of the Invention for a Compound of Formula 1 can be prepared as described in Scheme 5,

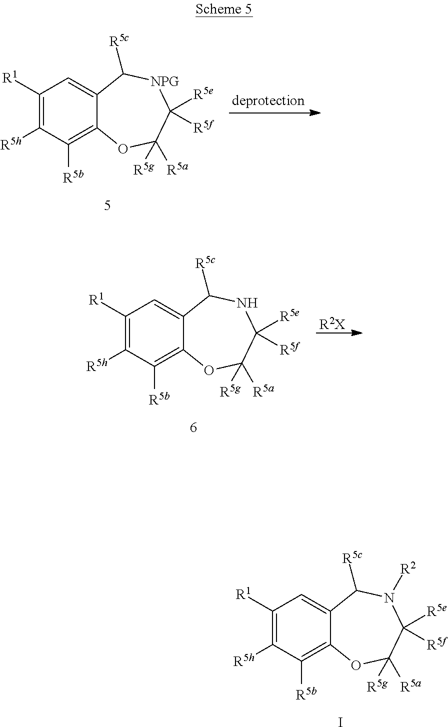

where X is halo or hydroxy.

In particular, a Compound of Formula 1(w) where R^{5a} is hydrogen or alkyl. R^{5h} is hydrogen or halo, R^{5h} is (C₁₋₃)alkyl, and R¹ and R² are independently as defined in the Summary of the Invention for a Compound of Formula 1 can be prepared as described in Scheme 5a.

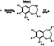

The protecting group on the intermediate of formula 5a is removed. When the protecting group is Boc, it can be removed with HCl to yield an intermediate of formula 6a. The intermediate of formula R²X (where X is a leaving group such as halo) is commercially available or can be prepared using procedures described herein or procedures known to one of ordinary skill in the art. The intermediate of formula 6a is then treated with R²X, in the presence of a base such as Hünig's base or NMP, in a solvent such as DMF, at a temperature of about 50° C. The product can be purified by column chromatography to yield an intermediate of Formula 1(w).

In particular, a Compound of Formula 1(a) where R¹, R², and R^{5h} are independently as defined in the Summary of the Invention for a Compound of Formula 1 can be prepared according to Scheme 5b.

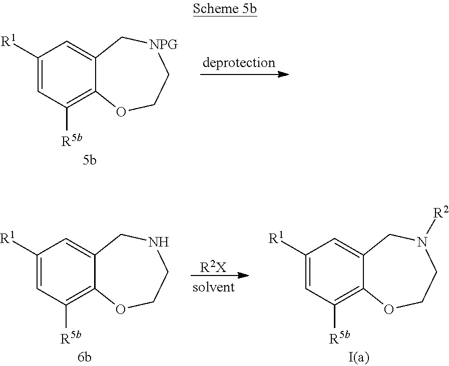

The protecting group on intermediate of formula 5b, prepared as described in Scheme 4a, is removed. When the protecting group is Boc, it can be removed with HCl to yield an intermediate of formula 6b. Intermediate 6b is then treated with an intermediate of formula R²X where X isa leaving group such as halo using standard alkylating conditions to yield a Compound of Formula 1(a).

A Compound of Formula 1(aa) where one of Y₁ and Y₂ is =CH- and the other is =N-, R¹ is benzimidazol-6-yl substituted at the 2-position with one R⁷; R^{5b}, R⁷ and R² are independently as defined in the Summary of the Invention for a Compound of Formula 1 can be prepared according to Scheme 6a using conditions known to one of ordinary skill in the art.

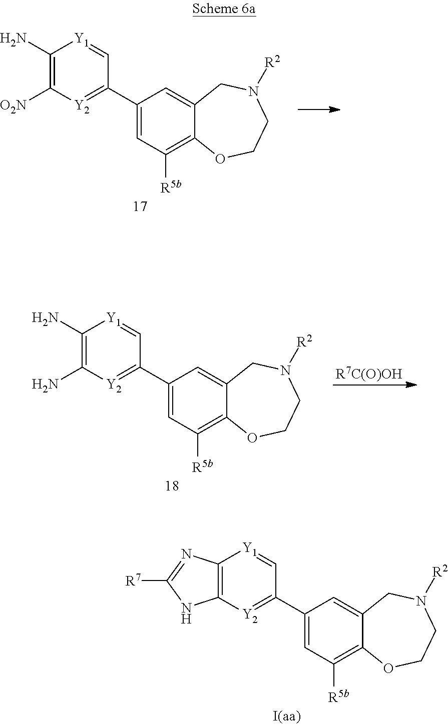

An intermediate of formula 17 is prepared by 1) treating an intermediate of formula 14a, prepared as described in Scheme 4a, with an intermediate of formula

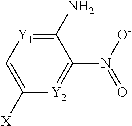

where X is halo using standard Suzuki coupling conditions; followed by 2) treating the with and intermediate of formula R²X using standard alkylating conditions, 17 is then hydrogenated in the presence of palladium on carbon in a solvent as acetic acid to yield the intermediate of formula 18, 18 is then treated with an acid of formula R⁷C(O)OH to yield the Compound of Formula 1(aa).

Alternatively, a Compound of Formula 1(aa) can be prepared according to Scheme 6b.

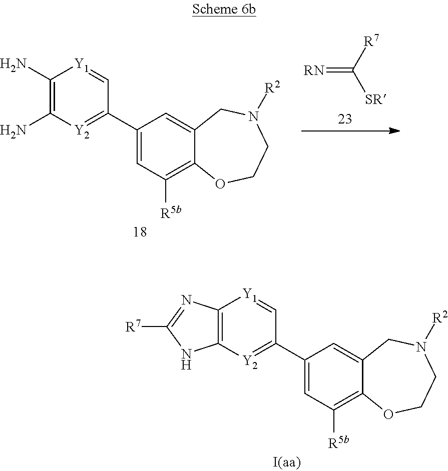

The intermediate of formula 18 is treated with an intermediate of formula 23 in the presence of glacial, acetic acid, optionally in the presence of triethyl orthoformate, and heated to yield an a Compound of Formula 1(aa).

A Compound of Formula 1(v) where R²and R^{5b} are as defined in the Summary of the Invention for a Compound of Formula 1 can be prepared according to Scheme 7a.

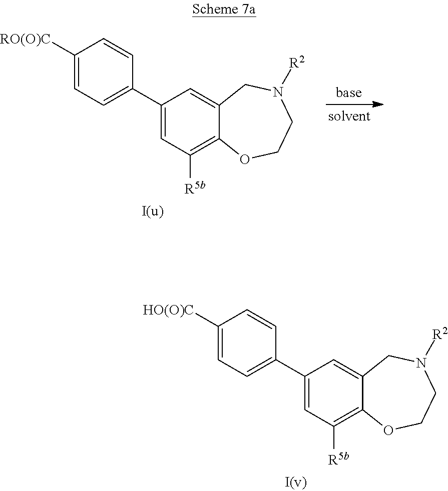

The Compound of Formula 1(u) where R is alkyl, prepared using procedures according to Scheme 5b, is treated with a base such as LiOH, in a solvent(s) such as THF and/or water to yield the hydrolyzed Compound of Formula 1(y).

A Compound of Formula 1(z) where R², R^{5b}, R⁸, and R^{8a} are independently as defined in the Summary of the Invention for a Compound of Formula 1 can be prepared according to Scheme 7b.

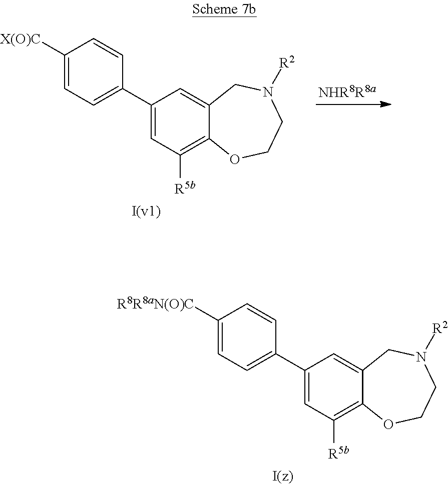

The Compound of Formula 1(v1) where X is halo or hydroxy can be prepared according to Scheme 7a or prepared by making the acid chloride from a Compound of Formula 1(v). The Compound of Formula 1(v1) is then treated with an amine of formula NHR⁸R^{8a} optionally in the presence of a base such as DIEA in a solvent such as THF to yield a Compound of Formula 1(z).

A Compound of Formula 1 where R¹, R², R^{5a}, R^{5b}, R^{5c}, R^{5d}, R⁵³, R^{5f}, R^{5g}, and R^{5h} are as defined above can be prepared according to the following scheme (where R is -B(OH)₂ and Y is halo, or R is halo and Y is -B(OH)₂) using Suzuki coupling procedures known to one of ordinary skill in the art.

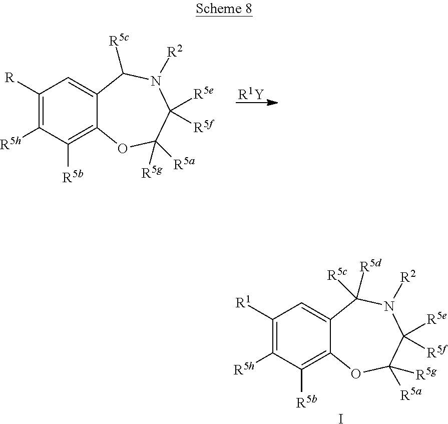

In particular, a Compound of Formula 1(a) where R¹, R^{5b}, and R² are independently as defined in the Summary of the Invention for a Compound of Formula 1 can be prepared as described in Scheme 8a.

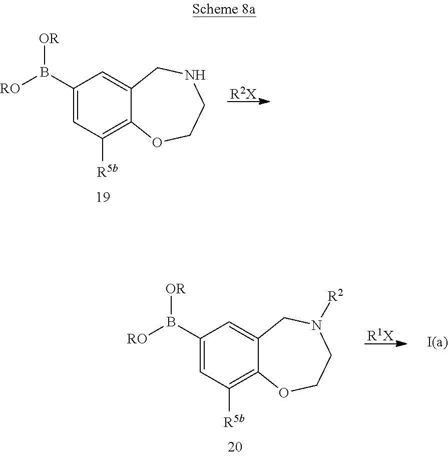

An intermediate of formula 19 (where each R is hydrogen or the two R's together forma boronic ester), which can be prepared by following step 1 of Scheme 4a and subsequent deprotection, is treated with an intermediate of formula R²X in a solvent such as dioxane/H₂O and in the presence of a base such as DIPEA. The resulting mixture is heated to about 90° C. to yield an intermediate of formula 20, 20 is treated with an intermediate of formula R¹X where X is halo and R¹ is as defined in ihe Summary of ihe Invention for a Compound of Formula 1 in a solvent such as DMF/water, in the presence of a base such as DIEA, in the presence of a catalyst such as (1,1′- bis(diphenylphosphino)ferrocene[dichloropalladium(11). The reaction is heated to about 95° C., 20 is then optionally purified to yield a Compound of Formula 1(a).

Alternatively, a Compound of Formula 1(a) where R¹, R^{5b}, and R² are independently as defined in the Summary of the Invention for a Compound of Formula 1 can be prepared as described in Scheme 8b.

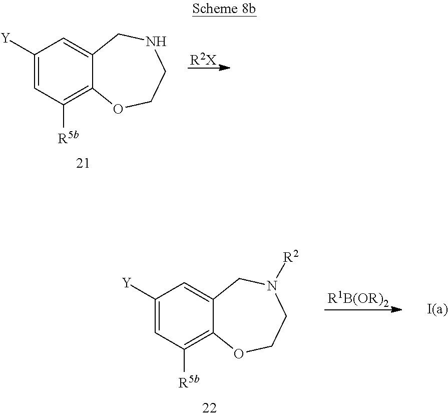

All intermediate of formula 21 where Y is halo, which can be prepared by following Scheme 1a followed by deprptection, is treated with an intermediate of formula R²X where X is halo, a base such as DIEA in a solvent such as 1-butanol and heated to yield an intermediate of formula 22, 22 is then treated with an intermediate of formula R¹B(OR)₂ (where each R is hydrogen or the two R together form a boronic ester), in the presence of a base such as potassium carbonate and in the presence of a catalyst such as dichloro[1,1 -bis(diphenyl- phosphino[ferrocenepalladium (11) dichloromethane adduct in a solveni such as dimethoxyethane/water. The reaction was heated and yielded a Compound of Formula 1(a).

### Synthetic Examples

### Reagent Preparation 1

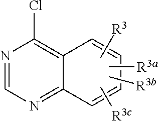

STEP 1: A solution of methyl 2-amino-5-bromo-4-methoxybenzoate (75 mg, 0.29 mmol) and ammonium formate (38 mg, 0.8 mmol) in formamide (1 mL) was heated at 165° C. for 18h. The mixture was allowed to cool to room temperature then diluted with an excess of water. The solid formed was collected by filtration and washed with water then ethyl acetate and dried to give 6-broma-7-methoxyquinazolin-4(3H)-one (53 mg, 72% yield) as a pale yellow solid. MS (EI) for C₉H₇BrN₂O₂: 255, 257 (MH⁺).

STEP 2:6-bromo-7-methoxyquinazolin-4(3H)-one (53 mg, 0.21 mmol) was taken into thionyl chloride (1.5 mL) followed by addition of catalytic DMF. The mixture was heated to 80° C. for 2 h then concentrated. The residue was partitioned with ethyl acetate and saturated aqueous sodium bicarbonate. The organic phase was washed with brine then dried over anhydrous sodium sulfate, filtered and concentrated to give 6-bromo-4-chloro-7- methoxyquinazoline (36 mg, 62 % yield) as a brown solid. MS (EI) for C₉H₆BrCln₂O: 275 (MH⁺).

Using analogous synthetic techniques and substituting with alternative starting reagents in step 1 the following reagents were prepared.

4-chloro-7-(methylsulfonyl)quinazoline. Synthesized according to the method of reagent preparation 1 using 7-(methylsulfonyl)quinazolin-4(3H)-one in step 2. ¹H NMR (400 MHz, d₆-DMSO): 8.36 (d, 1H), 8.34 (s, 1H), 8.18 (d, 1H) (dd, 1H), 3.36 (s. 3H).

4,7-dichloro-6-iodoquinazoline. Synthesized according to the method of reagent preparation 1 using methyl 2-amino-4-chloro-5-iodobenzoate in step 1. MS (EI) for C₈H₃Cl₂: 325 (MM⁺).

4-chloro-6-iodo-8-methylquinazoline.. Synthesized according to the method of reagent preparation 1 using 2-amino-5-iodo-3-methylbenzoic acid in step 1. MS (EI) for C₉H₆C11N₂: 305 (MM⁺).

4-chloro-6-(phenylmethoxy)-quinazoline. Prepared according to the method of reagent preparation 1 using 2-amino-5-benzyloxybenzoate acid methyl ester (J. Org. Chem. 2001, 66(8), 2784-2788) in step 1. MS (EI) for C₁₅H₁₁ClN₂O: 271 (MH⁺).

4.6-dichloro-7-methoxy-quinazoline. Prepared according to the method of reagent preparation 1 using 5-chloro-4-methoxyanthranilic acid (US 80-126838) in step 1. MS (EI) for C₉H₆Cl₂N₂O: 271 (MH⁺).

4-chloro-7,8-dimethoxy-qiuinazoline. Prepared according to the method of reagent preparation 1 using 2-amino-3,4-dimethoxybenzoic acid methyl ester (US 4287341) in step 1.MS (EI) for C₁₀H₉ClN₂O₂: 225 (MH⁺).

7-(benzyloxy)-4-chloro-8-methoxyquinazoline. Prepared according to the method of reagent preparation 1 using 2-amino-3-methoxy-4-(phenylmethoxy)benzoic acid (J. Med. Chem.1992, 35(14), 2703-10) in step 1, MS (EI) for C₁₆H₁₃ClN₂O₂: 301(MH⁺).

4,6-dichloro-7,8;dimethoxyquinazoline. Prepared according to the method of reagent preparation 1 using 2-amino-5-chloro-3,4-dimethoxybenzoic acid (US 4287341) in step 1. MS (EI) for C₁₀H₈Cl₂N₂O₂: 260 (MH⁺).

6-bromo-4,7-dichloroquinazoline. Synthesized according to the method of reagent preparation 1 by using 2-amino-5-bromo-4-chlorobenzoic acid in step 1. MS (EI) for C₈H₃BrCl₂N₂: 277 (MH⁺).

4-chloro-6-iodo-7-methoxyquinazoline. Synthesized according to the method of reagent preparation 1 by N-iodosuccinimide iodination of methyl 2-amino-4- methoxybenzoate to give methyl 5-iodo-2-amino-4-methoxybenzoate then proceeding with step 1. ¹H NMR (400 MHz, CDCl₃): 8.97, (s, 1H), 8.75, 7.31 (s, 1H), 4.08 (s, 3H), GC-MS for C₉H₆C11N₂O: 319 (M⁺).

7-bromo-4-chloro-8-methoxyquinazoline and 7-bromo-4-chloro-6- methoxyquinazoline. Synthesized according to the method of reagent preparation 1 by nitration and hydrogenation of methyl 4-bromo-3-methoxybenzoate to give a separable mixture of methyl 4-bromo-3-methoxy-2-aminobenzoate and methyl 4-bromo-5-methoxy-2- aminobenzoate then proceeding with step 1 individually. 7-bromo-4-chloro-8- methoxyquinazoline: ¹H NMR (400 MHz, CDCI₃): 9.09 (s, 1H), 7.92 (d, 1H), 7.87 (d, 1H), 4.21 (s, 3H), G₉H₆BrClN₂O: 272 (M⁺). 7-bromo-4-chloro-6-methoxyquinazoline: ¹HI NMR (400 MHz, CDCl₃): 8.95, (s, 1H), 8.40 (d, 1H), 7.45 (d, 1H), 4.18 (s, 3H), GC-MS for G₉H₆BrClN₂O: 272 (M⁺).

8-bromo-4-chloro-6-methyl-quinazoline. Synthesized according to the method of reagent preparation 1 using 2-amino-3-bromo-5-methybenzoic acid in step 1. GC-MS (EI) for C₉H₆BrClN₂: 257 (M⁺).

4-chloro-6-(methylsulfonyl)quinazoline. Synthesized according to the method of reagent preparation 1 using 6-(methylsulfonyl)quinazolin-4(3H)-one in step 2, 6-(methylsulfonyl)quinazolin-4(3H)-one was obtained by the one step oxidation of 6-(methylthio)quinazolin-4(3H)-one (J. Med. Chem. 1983, 26(3), 420-5). MS (EI) for C₉H₇ClN₂O₂: 242 (M⁺).

### Reagent Preparation 2

### 4-chloro-5-methyl-6-(phenylmethyl)pyrimidine

Prepared from 4,6-dichloro-5-methylpyrimidine and benzyl zinc bromide (0.5 M solution in tetrahydrofuran) according to the procedure described in WO 2007/146824 as a colorless oil. ¹H NMR (400 MHz,. CDCl₃): 8.78 (s, 1H), 7.33-7.18 (m, 5H), 4.19 (s, 2H), 2.36 (s, 3H): MS (EI) for C₁₂H₁₁ClN₂: 219 (MH⁺).

Reagent Preparation 3: 4-chloro)-6,6-dimethyl-5,6,7,8-tetrahydroquinazoline

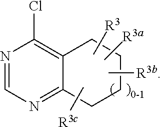

STEP 1: To a cooled (0° C.) solution of 4,4-dimethycyclohexanone (21 g, 0.17 mol) and dimethyl carbonate (45 g, 0.50 mol) in THF (400 mL) was added NaH (60% wt/wt in mineral oil. 17 g, 0.43 mol) portionwise over 30 minutes. The resulting slurry was allowed to stir at ambient temperature for 30 minutes followed by two hours at reflux. The reaction mixture was cooled (0° C.) and MeOH (30 mL) was added dropwise over 20 minutes. The resulting slurry was partitioned between 10% aqueous citric acid and ethyl acetate. The organic layer was washed with brine, dried over magnesium sulfate and concentrated in vacuo. Purification by vacuum distillation provided methyl 2-hydroxy-5,5-dimethylcyclohex- 1-enecarboxylate(22.5 g, 75% yield), ¹H NMR (400, MHz, CDCl₃) δ 12.15 (s, 1H), 3.75 (s, 3H), 2.29 (t, 2H), 2.03 (s, 2H), 1.44 (t, 2H), 0.96 (s, 6H); MS (EI) for C₁₀H₁₆O₃: 184 (M⁺)

STEP 2: A solution of methyl 2-hydroxy-5,5-dimethycyclohex-1-enecarboxylate (10.0 g, 54 mmol) and ammonium acetate (10 g, 130 mmol) ian ethanol (50 mL) was heated to reflux for 2 hours. The reaction was concentrated to onethird original volume, and then diluted withy ethyl acetate (100 mL). The organic solution was washed with water (100 mL) and brine (50 mL) and then dried over anhydrous sodium sulfate. After nitration and concentration, the residue was purified by silica gel column chromatography (ethyl acetate/hexanes, 1:8) to afford methyl 2-amino-5,5-dimethylcyclohex-1-enecarboxylate (7.42 g. 75% yield) as a yellow solid. MS (EI) for C₁₀H₁₇NO₂: 184 (MH⁺).

STEP 3: 2-amino-5m5-dimethylcyclohex-1 -enecarboxylate (7.42 g, 40mmol) was dissolved in N,N-dimethylformamide dimethylacetal (50 mL) and heated to 110° C. for 18 hours. The resulting solution was cooled to room temperature and concentrated to provide methyl 2-((dimethylamino)methyleneamino)-5,5-dimethycyclohex-1-enecarboxylate (9.5 g, 98% yield) as an oil. ¹H NMR (400 MHz, CDCl₃): 3,65 (s, 3H), 3.49 (s, 1H), 2.95 (s, 6H), 2.35 (m, 2H), 2.15 (br s. 2H), 1.41 (t. 2H). 0.95(s, 6H); MS (EI) for C₁₃H₂₂N₂O₂: 239 (MH⁺).

STEP 4: A solution of methyl 2((dimethylamino)methyleneamino)-5,5- dimethylcyclohex-1-enecarboxylate (9,5 g, 40 mol) in 7.0M ammonia in methanol (35 mL) was stirred at 25° C. for 90 minutes then concentrated to an oil. The residue was purified by silica gel column chromatography (ethyl acetate/hexanes, 1:8) to give 6,6-dimethyl 5,6,7,8- tetrahydroquinazolin-4(3H)-one (6.41 g, 90% yield) as a white solid. ¹H NMR (400 MHz, d₆- DMSO): 7.96 (s, 1H), 2.52 (t, 2H), 2.14(s, 2H), 1.48 (t, 2H), 0.93 (s, 6H); MS (EI) for C₁₀H₁₄N₂O: 179 (MH⁺).

STEP 5: To 6,6-dimethyl-5,6,7,8-tetrahydroquinazolin-4(3H)-one (6.41 g, 36 mmol) in chloroform (10 mL) added phosphorus oxychlqride (10 mL) and refluxed for 2 The mixture was concentrated to an oil, then diluted with ethyl acetate (80 mL) and washed with saturated sodium carbonate (50 mL) and brine (25 mL). The solution was dried over anhydrous sodium sulfate, filtered and concentrated, then the residue purified by silica gel column chromatography (ethyl acetate/hexanes, 1:8) to give 4-chloro-6,6-dimethyl- 5,6,7,8-tetrahydroquinazoline (5.3 g, 75% yield) as a yellow solid. ¹H NMR (400 MHz, CDCl₂): 8.72 (s, 1H), 2.52 (t, 2H), 2.14 (s, 2H), 1.48 (t, 2H), 0.93 (s, 6H): MS (EI) for C₁₀H₁₃ClN₂: 197 (MH⁺).

Using analogous synthetic techniques and substituting with alternative starting reagents in step 1 or 2 the following reagents were prepared. Alternative starting materials were available commercially unless otherwise indicated.

4-chloro-6-methyl-6,7-dihydro-5H-cyclopenta[d]pyrimidne. Prepared according to the method of reagent preparation 3; using 4-methyl-2-oxo-cyclopentanecarboxylic acid methyl ester (J. Chem. Soc. Perkin Trans I 1987, 7, 1485-8) in step 2. ¹H NMR (400 MHz, CDCI₃): 8.78 (s, 1H), 3.20 (m, 2H), 2.70(m, 3H), 1.22 (d, 3H), GC/MS (EI) for C₈H₉CIN_{2: 168} (M⁺).

4-chloro-6-cyclopropyl-5,6,7,8-tetrahydropyrido-[4,3-d]pyrimidine. Prepared according to the method of reagent preparation 3 using 1-cyclopropyl-4-oxo-3- piperdinecarboxylic acid methyl ester (Heterocycles, 1999, 50(2), 867-874) in step 2. ¹H NMR (400 MHz, CDcl₃): 8.78 (s, 1H), 3.79 (s, 2H), 2.98 (m, 4H), 1.88 (m, 1H), 0.60(m, 2H), 0.54 (m, 2H), MS (EI) for C₁₀H₁₂ClN₃: 210 (MH⁺).

4-chloro-6-cyclopropyl-6,7-dihydro-5H-pyrrolo[3,4-d]primidine. Prepared according to the method of reagent preparation 3 using 1-cyclopropyl-4-oxo-3- pyrrolidinecarboxylic acid methyl ester in step 2. MS (EI) for C₉H₁₀H_{ClN}₃: 1.96 (MH⁺).

4-chloro-6-p-tolyl-6,7-dihydro-5H-pyrrolo[3,4-d]pyrimidine. Prepared according to the method of reagent preparation 3 using l-(4-methylphenyl)-4-oxo-3- pyrrolidinecarboxilic acid ethyl ester in step 2. ¹H NMR (400 MHz, CDCl₃): 8.92 (s, 1H), 7.14 (d, 2H), 6.62 (d, 2H), 4.70 (m, 4H), 2.30 (s, 3H), MS (EI) for C₁₃H₁₂ClN₃: 246 (MH⁺).

4-chloro-7-methyl-7-phenyl-5,6,7,8-tetrahydroquinazoline. Prepared according to the method of reagent preparation 3 using 4-methyi-2-oxo-4-phenyl cyclohexanecarboxylic acid methyl ester (J. Org. Chem. 1991, 56(21), 6199-205) in step 1 EMS (EI) for C₅H₁₅ClN₂: 259 (MH⁺).

4-chloro-5-phenyl-6,7-dihydro-5H-cyclopenta[d]pyrimidine. Synthesized according to the method of reagent preparation 3 using ethyl 2-oxo-5- phenylcyclopentanecarboxylate in step 2. MS (EI) for C₁₃H₁₁ClN₂: 231 (MH⁺).

4-chloro-7,7-dimethyl-5,6,7,8-tetrrahydroquinazoline: Synthesized according to the method of reagent preparation 3 using ethyl 4,4-dimethyl-2-oxoeyclohexanecarboxylate in step 2. ¹H NMR (400 MHz, CDCl₃): 8.91 (s, 1H), 2.90 (s, 2H), 2.88 (tr, 2H), 1.73 (tr, 2H), 1.07 (s, 6H); MS (EI), for C₁₀H₁₃ClN₂: 197 (MH⁺).

4′-chloro-7′,8′-dihydro-5′H-spiro[cyclopropane-1,6′-quinazoline]. Prepared according to the method of reagent preparation 3 using spiro[2,5]octan-6-one in step 1. ¹H NMR (400 MHz, CDCl₃) δ 8.73 (s, 1H), 3.00 (t, 2H), 2.63 (s, 2H), 1.69 (t, 2H), 0.52 (s, 4H): MS (EI) for C₁₀H₁₁ClN₂: 194 (M⁺).

b 4-chloro-6,6-difluoro-5,6,7,8-tetrahydroquinazolline. Synthesized according to the method of reagent preparation 3 using 4,4-difluorocyclohexanone in step 1. MS (EI) for C₈H₇Clf₂N₂: 204 (M⁺).

(R)-4-chloro)-7-methyl-5,6,7,8-tetrahydroquinazoline. Synthesized according to the method of reagent preparation 3 using (R)-3-methylcyclohexanone in step 1. MS (EI) for C₉H₁₁ClN₂: 182 (M⁺).

4-chloro-2,6-dimethyl-5,6,7,8-tetrahydroquinazoline. Synthesized according to the method of reagent preparation 3 using 4-methylcyclohexanone in step 1 and 1,1-dimethoxy- N,N-dimethylethanamine in step 3. MS(EI) for C₁₀H₁₃ClN₂: 196 (M⁺).

4rchlqro-6-ethyl-2-mniethyl-5,.6,7,8-tetrahydroquinazoline. Syndesozed according to the method of reagent preparation 3 using 4-ethylcyclohexanone in step 1 and 1.1-dimethoxy-N,N-dimethylethanamine in step 3. MS (EI) for C₁₁H₁₅ClN₂: 210 (M⁺).

4-chloro-7-(trifluoromethyl)-5,6,7,8-tetrahydroquinazoline. Synthesized according to the method of reagent preparation 3 using methyl 2-hydroxy-4-(trifluormethyl)cyclohex-1- enecarboxylate in step 2. MS (EI) for C₉H₈ClF₃N₂: 236 (M⁺).

(trans)-chloro-6,7-dimethyl-5,6,7,8-tetrahydroquinazoline. Synthesized according to the method of reagent preparation 3 using (trans) 3,4-dimethycyclohexanone in step 1. MS (EI) for C₁₀H₁₃ClN₂: 1.96 (M⁺).

4-chloro-6-(trifluoromcehyl)-5,6,7,8-tetrahydroquinaoline. Synthesized according to the method of reagent preparation 3 using 4-(trfluormethyl )cyclohcxanone in step 1. MS (EI) for C₉H₈ClF₃N₂: 236 (M⁺).

(S)-4-chloro-7-methyl-5,6,7,8-tetrahydroquinazoline. Synthesized according to the method of reagent preparation 3 using (S)-3-methyleyclohexanone (US 20060293364) in step 1. MS (EI) for C₉H₁₁ClN₂: 182 (M⁺).

4-chloro-5-(trfluoromethyl)-5,6,7,8-tetrahydroquinazoline. Synthesized according to the method of reagent preparation 3 using methyl 2-hydroxy-6-(trifluormethyl)cyclohex-1- enecarboxylate in step 2. MS (EI) for C₉H₈ClF₃N₂: 236 (M⁺).

4-chloro-7-vinyl-5,6,7,8-tetrahydroquinazoline. Synthesized according to the method of reagent preparation 3 using 3-vinylcyclohexanone (J. Med. Chem. 1.987, 30, 1177- 1186) in step 1. MS (EI) for ₁₀H₁₁ClN₂: 194 (M⁺).

4-chloro-8,8-dimethyl-5,6,7,8tetrahydroquinazoline. Synthesized according to the method of reagent preparation3 using 2,2-dimethylcyclohexanone in step 1. MS (EI) for C₁₀H₁₃ClN₂: 196 (M⁺).

4-chloro-6,6,7-trimethyl-5,6-dihydroquinazoline. Synthesized according to the method of reagent preparation 3 using 3,4,4-trimethylcyclohex-2-enone (J. Am. Chem. Soc. 1994, 116, 2902-2913) in step 1. MS (EI) for C₁₁H₁₃ClN₂: 208 (M⁺).

(S)-4-chloro-8-vinyl-6,7,8,9-tetrahydro-5H-cyclohepta[d]pyrmidine. Synthesized according to the method of reagent preparation 3 using (S)-3-vinylcycloheptanone (prepared using procedure for(S)-3-vinylcyclohexanone in Org. Lett. 2003, 5, 97-99, but starting with (Z)-cyclohept-2-enone) in step 1. MS (EI) for C₁₁H₁₃ClN₂: 208 (M⁺).

4-chloro-6,6-dimethyl-5-dihydroquinazoline. Synthesized according to the method of reagent preparation 3 using 4,4-dimethylcyclohex-2-enone in step 1. MS (ES) for C₁₀H₁₁ClN₂: 195 (MH⁺).

4-chloro-6,6,8-trimethyl-5,6-dihydroquinazoline. Synthesized according to the method of reagent preparation 3 using 2,4,4-trmethylcyclohex-2-enone in step 1. MS (EI) for C₁₀H₁₁ClN₂: 209 (MH⁺).

4-chloro-6,6,7,8-tetramethyl-5-dihydroquinazoline. Synthesized according to the method, of reagent preparation 3 using 2,3, 4,4-trimethylcyclohex-2-enone (J. Org. Chem. 1981, 46, 1515-1521 ) in step 1. MS (EI) for C₁₂H₁₅ClN₂: 223 (MH⁺).

(S)-4-chloro-7-ethyl-5,6,7,8-tetrahydroquinazoline. SyntliesizedaCcording to the method of reagent preparation 3 using (S)-3-ethylcyclohexanone (Tetrahedron: Asymmetry, 1997, 8, 1253-1257) in step 1. MS (EI) for C₁₀H₁₃ClN₂: 197 (MH⁺).

### Reagent Preparation 4

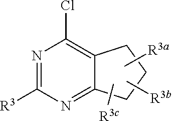

Step 1: A solution of methyl 4-methyl-2-oxocyclopentanecarboxylate (0.42 g, 2.69 mmol), 2-methyl-2-thiopseudourea sulfate (1.10 g, 7.9 mmol) and potassium hydroxide (0.50 g, 8.9 mmol) in water (12 mL) was stirred at 25° C. for3D minutes, and then heated to reflux for 4 hours. The reaction was cooled to 0° C. by adding ice and a precipitate was formed. The solid product was removed by filtration and the filler cake dried to give 6- methyl-2-(methylthio)-6,7-dihydro-3H-cyclopenta[d]pyrimidin-4(5H)-one (0.19 g. 43% yield) as a white solid. (¹H NMR (400 MHz, d6-DMSG): 2.87 (m, 2H). 2.53 (s. 3H), 237 (m, 2H), 2.28 (s, 3H), 1.49 (m, 1H), 1.,02 (d, 3H).

Step 2: A solution of 6-methyl-2-(methylthio)-6,7dihydro-3H- cyclopenta[d]pyrimidin-4(5H)-one (0.19 g, 0.97 mmol) in phosphorous oxychloride (5.0 mL) was heated to 95° C. for 1 hour. After cooling the reaction was concentrated, and the residue dissolved in ethyl, acetate (50 mL) and washed with cold water (25 mL), 0.1 M aqueous sodium hydroxide (25 mL) and brine (20 mL). The organic phase was dried over anhydrous sodium sulfate, filtered and concentrated. The residue was chromatographed on silica gel (diethyl eiher/hexanes, 1:10) and the product containing fractions concentrated. The residue thus obtained was purified further by preparative reverse phase WPLC (0.1% aquepus ammonium acetate-acetonitrile to give 4-chloro-6-methyl-2-(methylthio)-6,7-dihydro-5H- cyelopenta[d]pyrimidine (25 mg, 12% yield) as an oil. ¹H NMR (400 MHz, d₆-DMSO); 3.12 (m, 2H), 2.61 (m, 2H), 2.56 (s, 3H), 1.25 (m, 1H), 1.18 (d, 3H); MS (EI) forC₉H₁₁ClN₂S: 215(MH⁺).

Using analogous synthetic techniques and substituting with alternative starting reagents the following reagents were prepared. 4-chloro-2-(methylthio)-6,7-dihydro-5H-cyclopenta[d]pyrimidine. Synthesized according to the method of reagent preparation 4 by replacement of step 1 with l,2,3,4,5,6,7- hexahydro-2-thioxo-4H-cyclopentapyrimidin-4-one S-alkylation with iodomethane and proceeding to step 2. ¹H NMR (400 MHz, CDCl₃): 3.00 (tr. 2H), 2.92 (tr, 2H), 2.56 (s, 3H), 2.14 (m, 2H).

2-(benzylthio)4-chloro-6,7dihydro-5H-cyclopenta[d]pyrimidine. Synthesized according to the method of reagent preparation 4 by replacement of step 1 with 1,2,3,5,6,7- hexahydro-2-thioxo-4H-cyclopentapyrimidin4-one S-alkylation with benzyl bromide and proceeding to step 2. ¹H NMR (400 MHz, CDCl₃): 7.43 (d, 2H), 7.27 (tr, 2H), 7.22-7.18 (m, 1H), 4.38 (s, 2H), 2.95 (tr, 2H), 2.86 (tr, 2H), 2.08 (m, 2H).

4-chloro-2-(ethylthio)-6,7-dihydro-5H-cyclopcenta[d]pyrimidine. Synthesized according to the method of reagent preparation 4 by replacement of step 1 with 1,2,3,5,6,7- hexahydro-2-thioxo-4H-cyclopentapyrimidin-4-oneS-alkylation with iodoethane and proceeding to step 2. ¹H NMR,(400 MHz, CDCl₁); 3.08 (q, 2H), 2.93 (tr, 2H), 2.86 (tr, 2H), 2.08 (m, 2H), 1.32 (tr, 3H).

### Reagent Preparation 5

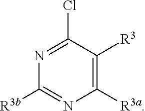

STEP 1: A solution of ethyl 4-methyl-3-oxopentanoate (3.0 g, 19.0 mmol) and potassium carbonate (7.86 g, 56.9 mmol) in THF (40 mL) was stirred at room temperature for 3 h under N₂ (g). The mixture was cooled to 0° C. and methyl iodide (3.23 g, 22.8 mmol) was added dropwise over 5 min. The reaction mixture was allowed to warm to room temperature and stirred for 16 h. Subsequent filtration and concentration provided ethyl 2.4-dimethyl-3- oxopentanoate-(2.89 g, 89% yield) as a clear yellow oil that was used without further purification. MS (EI) for C₉H₁₆O₃: 172 (MH⁺).

STEP 2: To anhydrous ethanol (110 mL) was added sodium metal (1.16 g, 50.4 mmol) and the mixture was stirred until dissolution was complete. To this solution was added thiourea (1.79 g, 23.5 mmol) and ethyl 2,4-dimethyl-3-oxopentanoate (2.89 g, 16. 8 mmol). The reaction mixture was stirred at 85° C. for 20 h then cooled and concentrated. The residue was diluted with water, the pH adjusted, to 4 with 1 N hydrochloric acid then extracted with ethyl acetate (3×80 mL). The combined organic layers were washed with brine, dried over anhydrous sodium sulfate, filtered and concentrated to provide 6-isopropyl-5-methyl-2- thioxo-2,3-dihydropyrimidin-4(1H)-one (2.40 g, 78% yield) as a tan solid that was used without further purification. C₈H₁₂N₂OS: 185 (MH⁺).

STEP 3: To a solution of 30% hydrogen peroxide (12 mL) and water (23 mL) was slowly added 6-isopropyl-5-methyl-2-thioxo-2,3-dihydropyimidin-4(1H)-one (1.0 g, 5.4 mmol). The reaction mixture was stirred at 70° C. for 3 h. After cooling to room temperature, saturated sodium carbonate was slowly added until the pH reached 10. To this mixture was slowly added a 1 M solution of sodium thiosulfate until residual peroxide was quenched, whereupon the aqueous solution was concentrated to dryness. The residue was suspended in chloroform (100 mL), filtered to remove inorganic salts and the filtrate concentrated to provide 6-isopropyl-5-methylpyrimidin-4-ol (0.25 g, 30% yield) as a white solid that was used without further purification. MS (EI) for C₈H₁₂N₂O: 153 (MH⁺).

STEP 4: To 6-isopropyl-5-methylpyrimidin-4-ol (0.25 g, 1.6 mmol) was added neat phosphorous oxychloride (5 mL) and the mixture stirred at 70° C. for 3 h. After cooling to room temperature the solution was concentrated, diluted with water then neutralized by portionwise addition of saturated sodium carbonate solution. The aqueous mixture was extracted with ethyl acetate and the organic solution washed with brine then dried over anhydrous sodium sulfate. Filtration and concentration provided 4-chloro-6-isopropyl-5- methylpyrimidine (30 mg, 11% yield) as a brown oil that was used without further purification. MS (EI) for C₈H₁₁ClN₂: 170 (MH⁺).

Using analogous synthetic techniques and substituting with alternative starting reagents in step 1 the following reagents were prepared.

4-chloro-5-(cyclopropylmethyl)-6-methylpyrimidine. Syhthesized accbrding to the method of reagent preparation 5 using methyl 3-oxobutanoate and (bromomethyl)cyclopropane in step 1. MS (EI) for C₉H₁₁ClN₂: 182 (MH).

4-chloro-5-(4-chlorobenzyl)-6-methylpyrimidine. Synthesized accorind to the method of reagent preparation 5 using methyl 3-oxobutanoate and 1-(bromomethyl)-4- chlorobenzene in step 1. MS (EI) for C₁₂H₁₀Cl₂N₂: 254 (MH⁺).

4-chloro-5-(3,5-difluorobenzyl)-6-methylpyrimidine. Synthesized according to the method of reagent preparation 5 using, methyl 3-oxobutanoate and 1 -(bromqmethyl)-3,5- difluorobenzene in step 1. MS (EI) for C₁₂H₉ClF₂N₂: 255 (MH⁺).

4-chloro-6-methyl-5-(3-(trifluoromethyl)benzyl)pyrimidine. Synthesized according to the method of reagent preparation 5 using methyl 3-oxoutanoate and 1-(chloromethyl)-3-(trifluoromethyl)benzene in step 1. MS (EI) for C₁₃H₁₀ClF₃N₂: 287 (MH⁺).

4-chloro-5-(1-(3-fluorophenyl)ethyl)-6-methylpyrimidine. Synthesized according to the method of reagent preparations using methyl 3-oxobutanoate and 1-(3- fluorophenyl)ethyl methanesulfonate in step 1. MS (EI) for G₁₃H₁₂ClFN₂: 251 (MH⁺).

4-chloro-5-(4-chloro-3-fluorobenzyl)-6-methylpyrimidine. Synthesized according to the method of reagent preparation 5 using- methyl-3-oxobutanoate and 4-(bromomethyl)-1 - chloro-2-fluorobenzene in ste 1. MS (EI) for C₁₂H₉Cl₂FN₂: 272 (MH⁺).

4-chloro-5-(4-fluorobenzyl)-6-methylpyrimidine. Synthesized according to the method of reagent preparation 5 using methyl, 3-oxobutanoate and 1-(bromomethyl)-4- fluorobenzene in step 1. MS (EI) for C₁₂H₁₀ClFN₂: 237 (MH⁺). 3

4-chloro-5-(2-fluorobenzyl)-6-methylpyrimidine. Prepared according to the method of reagent preparation 5 by using methyl 3-oxobutanoate and 1-(bromomethyl)-2- fluorobenzene in step 1¹H NMR (400 MHz, CDCI₃): 8.79 (1H), 7.28 to 7.12 (m, 1H), 7.14 to 6.97 (m, 2H), 6.82 (dd, 1H), 4.19 (s, 2H), 2.47 (s,.3H), GC-MS for C₁₂H₁₀ClFN₂: 236 (M⁺).

4-chloro-5-ethyl-6-isopropylpyrimidine. Prepared according to reagent preparation 5 by using ethyl isobutyrylacetate and iodoethane in step 1. MS (EI) for C₉H₁₃ClN₂: 184(M⁺).

5-benzyl-4-chloro-6-methylpyrimidine. Prepared according to reagent preparation 5 by using ethyl 2-benzylacetoacetate in step 2. MS (EI) for C₁₂H₁₁ClN₂: 219 (MH⁺).

4-chloro-6-ethyl-5-methyl-pyrimidine. Prepared according to reagent preparation 5 by using methyl3-oxopentanoate in step 1. ¹H NMR (400 MHz, CDCl₃): 8.74 (s, 1H), 2.85 (q, 2H), 2.39 (s, 3H), 1.30 (t, 3H), MS (EI) for C₇H₉ClN₂: 158 (MH⁺).

4-chloro-5,6,7,8-tetrahydroquinazoline. Synthesized according to the method of reagent preparation 5 using ethyl 2-oxocyclohexanecarboxytate in step 2^{. 1}H NMR (400 MHz, CDCl₃); 8.7 (s, 1H), 2.90 (m, 2H), 2.78 (m, 2H), 1.88 (m, 4H), MS (EI) for C₈H₉ClN₂: 169 (MH⁺).

4-chloro-5,6-diethyl-pyrimidine. Prepared according to reagent preparation 5 by using methyl 3-oxopentanoate and iodoethane in step 1.

4-chloro-6-methyl-5-(1-methylethyl)-pyrimidine. Prepared according to reagent preparation 5 by using methyl 3-oxobutanoate and 24-iodopropane in step 1. ¹HNMR (400 MHz, PMSO-d₆): 8.70 (s, 1H), 3.49 (h, 1H), 2.60 (s, 3H), 1.34 (d, 6H); MS (EI) for C₈H₁₁ClN₂: 171 (MH⁺).

4-chloro-5-isobutyl-6-methylpyrimidine. Prepared according to reagent preparation 5 by using methyl 3-oxobutanoate and 1-odo-2-methylpropane in step 1. MS (EI) for C₉H₁₃ClN₂: 184 (M⁺).

5-benzyl-4-chloro-6-ethylpyrimidine. Prepared according to reagent preparation 5by using methyl 3-oxopentanoate and benzyl bromide in step 1, ¹H NMR (400 MHz, CDCl₃): 8.83 (s, 1H), 7.27 (m, 3H), 7.08 (m, 2H), 4.22 (s, 2H) , 2.79 (q, 2H), 1.20 (t, 3H); MS (EI) for C₁₃H₁₃ClN₂: 234 (MH⁺).

4-chloro-5-(3-fluorobennzyl-6-methylpyrimidine. Prepared according to reagent preparation-5 by using methyl 3-oxobutanoate and 3-fluorobenzylbromide in step 1. MS (EI) for C₁₂H₁₀ClFN₂: 237 (MH⁺).

4-chloro-5-(3-chlorobenzyl)-6-methylpyrimidine. Prepared according to reagent preparation 5 by using methyl 3-oxobutanoate and 3-chlorpbenzylbromide in step 1. MS (EI) for C₁₂H₁₀Cl₂N_{2: 253} (MH⁺).

4-chloro-6-methyl-5-phenoxy-pyrimidine. Prepared acccrding to reagent preparation 5 by using ethyl 3oxo-2phenoxybutanoate in Step 2. MS (EI) for C₁₁H₉ClN₂O: 221 (MH⁺).

4-chloro-6-methyl-5-(1 -phenyl)pyrimidine. Prepared according to reagent preparation 5 by using methyl 3-oxobulanoate and (1-bromoethyl)benzene in step 1. MS (EI) for C₁₃H₁₃ClN₂: 233 (MH⁺).

4-chloro-5-(2-chlorobenzyl)-6-methylpyrimidine. Prepared according to reagent preparation 5 by using methyl 3-oxobutanoate and 2-chlorobenzyl bromide in step 1.

4-chloro-6-methyl-5-(4-methylbenzyl)pyrimidine. Prepared according to reagent preparation 5 by using methyl 3-oxobutanoate and 4-methylbeuzyl bromide in step 1. ¹H NMR (400 MHz, CDCl₃): 8.76 (s, 1H), 7.10 (d, 2H), 6.99 (d, 2H), 4.15(s, 2H), 2.50 (s, 3H), 2.32 (s, 3H); MS (EI) for C₁₃H₁₃ClN_{2:} 233 (MH⁺).

4-chloro-5-(4-methoxybenzyl)-6-methylpyrimidine. Prepared according to reagent preparation 5 by using methyl 3-oxobutanoate and 4-methoxybenzyl bromide in step 1. ¹H NMR (400 MHz, CDCl₃): 8.76 (s, 1H), 7.02 (d, 2H), 6.83 (d, 2H), 4.13 (s, 2H), 3.78 (s, 3H) 2.5 (s, 3H); MS (EI) for C₁₃H₁₃ClN₂O: 249 (MH⁺).

4-chloro-5-(3-methoxybenzyl)-6-methylpyrimidine. Prepared accroding to reagent preparation 5 by using methyl 3-oxobutanoate and 3-menethoxybenzyl bromide in step 1. ¹H NMR (400 MHz, DMSO-d₆): 8.81 (s, 1H), 7.22 (m, 1H), 6.81 (m, 1H), 6.70 (s, 1H), 6.63 (d, 1H), 4.17 (s, 2H), 3.71 (s, 3H), 2.47 (s, 3H); MS (EI), for C₁₃H₁₃ClN₂O: 249 (MH⁺).

4-chloro-6-methyl-5-(3-methylbenzyl)pyrimidine. Prepared according to reagent preparation 5 by using methyl 3-oxobutanoate and 3-methylbenzyl bromide in step 1. ¹H NMR (400 MHz, CDCl₃): 8.77 (s, 1H), 7.18 (m, 1H) 7.05 (d, 1H), 6.88 (m, 2H), 4.16 (s, 2H), 2.50 (s, 3H), 2.31 (s, 3H); :MS (EI) for C₁₃H₁₃ClN₂: 233 (MH⁺);

5-benzyl-4-chloropyrimidine. Prepared according g to reagent preparation 5 by using ethyl 2-benzyl-3-hydroxyacrylate (J. Am. Chem. Soc. 1974, 96, 2121-2129) in step 2. MS (EI) for C₁₁H₁₃ ClN₂: 205 (MH⁺).

4-chloro-5-(3-chloro-5-fluorbenzyl)-6-methylpyrimidine. Prepared according to reagent preparation 5 by using methyl 3-oxobutanoate and 3-chloro-5-fluorobenzyl bromide in step 1. MS (EI) for C₁₂H₉Cl₂FN₂: 271 (MH⁺).

4-chloro-5-(2-methoxybenzyl)-6-methylpyrimidine. Prepared according to reagent preparation 5 by using methyl 3-oxobutanoate and 2-methoxylbenzyl in step 1. ¹H NMR (400 MHz; methanol-d₄): 8.71 (s, 1H), 7.23 (m, 1H), 6.98 (d, 1H), 6.83 (m, 1H), 6.71 (d, 1H), 4.16 (s, 2H), 3.85 (s, 3H); 2.45 (s, 3H),

4-chloro-6-methyl-5-(2-methylbenzyl)pyrimidine. Prepared according to reagent preparation 5 by using methyl 3-oxobultanoate and 2-methylbenzyl bromide in step 1. ¹H NMR (400 MHz. mclhanol-d.1): 8.77 (s. lH). 7.23 (d. Ill), 7.12 (m. li-l). 7.03 (m, 1H). 6.45(d, 1H), 4.16 (s, 2H), 2.43 (s, 3H), 2.42 (s, 3H).

4-chloro-5-(3,4-difluorobenzyl)-6-methylpyrimidine. Prepared according to reagent preparation 5 by using methyl 3-oxobutanoate and 3,4-difluorobenzyl bromide in step 1. MS (EI)for C₁₂H₉ClF₂N₂: 255 .(MM*).

4-chloro-6-methyl-5-(4-(trifluoromethyl)benzy)pyrimidine. Prepared according to reagent preparation 5 by using methyl 3-oxobutanoate and 1-(chloromethyl)-4-(trifluoro- methyl)benzene in step 1. MS (EI) for C ₁₃H₁₀ClF₃N₂: 287 (MH⁺).

5-benzyl-4-chloro-6-(trifuoromethyl)pyrimidine. Prepared according to reagent preparation 5 by using ethyl 4,4,4-trifluoroacetoacelate and benzyl bromide in step 1. MS (EI) for C₁₂H₈ClF₃N₂: 272 (M⁺).

4-chloro-6,6-dimethyl-6,7-dihydro-5H-cyclopena[d]pyrimidine. Synthesized according to the method of reagent preparation 5 using eyhyl 4,4-dimethyl-2-oxo- cyclopentanecarboxylate in step 2. MS (EI) for C₆H₁₁ClN₂: 183 (MH⁺).

### Reagent Preparation 6

### 6-chloro-5-methyl-N-phenylpyrimidin-4-amine

STEP 1: To a mixture of 4,6-dichloro-5-methylpyrimidine (2.27g, 3.9 mmol) and aniline (1.0 g, 10.7 mmol) in isopropanol (15mL) was added concentrated aqueous hydrochloric acid (1.5 mL) and heated to reflux for 2.5 h. The mixture was then concentrated and the residue triturated with ethyl acetate:isopropanol 4:1, The solid was collected by filtration and washed with additional ethyl acetate:isopropanol 4:1 then dried lo give 6- chloro-5-methyl-N-phenylpyrimidin-4-amine (2.0 g, 67% yield), ¹ H NMR (400 MHz, d₆- DMSO): 8.85 (s, 1H), 8.26 (s, 1H), 7.60 (d, 2H), 7.35 (tr, 2H), 7.11 (tr, 1H), 2.31 (s, 3H), MS (EI) for C₁₁H₁₀ClN₃: 220 (MH⁺).

### Reagent Preparation;8

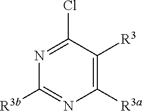

STEP 1: To a suspension of potassium tert-butoxide (10.6 g, 95.0 mmol) in tetrahydrofuran (100 mL) were added methyl accloacelate (10.0 g, 86.0 mmol) and tert-butanol (0.83 mL, 8.6 mmol) at room temperature. The resulting solution was stirred for 1 h, and then 4-fluorobenzylbromide (11.2 mL, 90 mmol) was added. The reaction mixture was stirred at roomtemperattire for 18 h. and then partitioned between water and ethyl acetate. The aqueous layer was extracted with ethyl acetate (3 x), the combined organic extracts were washed with brine, dried over sodium sulfate, filtered and concentrated. Column chromatography of the residue on silica (5-20% ethyl acetate in hexanes) gave methyl 2-(4- fluorobenzyl)-3-oxobutanoate (14.5 g, 75% yield) as a colorless oil which was used in the next step without further purification.

STEP 2: To a suspension of acetamidine hydrochloride (0.54 g, 5.71 mmol) in methanol (8 mL) was added a 30% solution of sodium methoxide in methanol (1.1 mL, 5.7 mmol), and the resulting solution was stirred at room temperature for 45 min. Then, a solution of methyl 2-(4-fluorobenzyl)-3-oxobutanoate (0.80 g, 3.57 mmol) in methanol (3 mL) was added dropwise, and the resulting mixture was stirred at room temperature for 22 h. Water (100 mL) was added, and the mixture was extracted, with chlorofotm (4×50 mL). The combined organic extracts were dried over sodiuim sulfate, filtered and concentrated to provide 5-(4-fluorobenzyl)-2,6-dimethylpyrimidin-4-ol (0.74 g, 89% yield) as a colorless solid. ¹H NMR (400 MH, methanol-d₆): 7.21 (m, 2H), 6.96 (m, 2H), 3.84 (s, 2H), 2.35 (s, 3H), 2.25 (s, 3H): MS (EI), for C₁₃H₁₃FN₂O: 233 (MH⁺).

STEP 3: A solution of 5-(44fluorobenzyl)-2.6-dirticthylpyritnidiii-4-or(730 nig, 3.14 mmol) in phosphorus oxychloride (10 mL) was stirred at 60° C. for 90 min. The reaction mixture was concentrated and ethyl acetate (50 mL) was added to the residue. The organic solution was washed with saturated sodium bicarbonate (50 mL), water (50 mL), and brine (50 mL), dried over sodium sulfate, filtered and concentrated. Column chromatography of the residue on silica (5-40% ethyl acetate in hexanes) afforded 4-chloro-5-(4-fluorobenzyl)-2,6- dimethylpyrimidine (527 mg, 6.7% yield) as a colorless solid. ¹H NMR (400 MHz, CDCl₃): 7.21 (m, 2H), 6.98 (, 2H), 4.12 (s, 2H), 2.67 (s, 3H), 2.45 (s, 3H); MS (EI) for C₁₃H₁₂ClFN₂: 250 (M⁺).

Using analogous synthetic technicques and substituting with alternative starting reagents in step 1 the following reagents were prepared.

4-Chloro-7-methyl-5,6,7,8-tetrahydroquinazoline. Prepared according to the method of reagent preparation 8 by using ethyl 4-methyl-2-oxocyclohexanecarboxylate and formamidine formate in step 2. GC-MS for C₁₀H₁₃ClN₂: 182 (M⁺).

4-Chloro-6-ethyl-5,6,7,8-tetrahydroquinazoline. Prepared according to the method of reagent preparation 8 by using methyl 5-ethyl-2-oxocyclohexanecarboxylate and formamidine formate in step 2. GC-MS for C₁₀H₁₃ClN₂: 196 (M⁺).

4-Chloro-5-ethyl-2,6-dimethylpyrimidine. Synthesized according to the method of reagent preparation 8 by using ethyliodide in step 1. MS (EI) for C₈H₁₁ClN₂: 171 (MH⁺).

4-Chloro-5-(cyclopropylmethyl)-2,6-dimethylpyrimidine. Synthesized according to the method of reagent preparation 8 by using cyclopropylmethylbromide in step 1. MS (EI) for C₁₀H₁₃CIN₂: 197 (MH+).

4—Chloro-2,6,6-trimethyl-5,6,78,-tetrahydroquinazoline. Synthesized according to the method of reagent preparation 8 by using methyl 5,5-dimethyl-2-oxocyclohexane- carboxylate in step 2. MS (EI) for C₁₁H₁₅ClN₂: 2.11 (MH⁺).

4-Chloro-6,6-dimethyl-2-(pyridin-2-yl)-5,6,7,8-tetrahydroquinazoline. Synthesized according to the method of reagent preparation 8 by using 2-hydroxy-5,5- dimethylcyclohex-1-enecarboxylate and picolinimidamide hydrochloride in step 2. MS (ES) for C₁₅H₁₆ClN₃: 274(MH⁺).

2-(4-chloro-6,6-dimethyl-56,7,8-tetrahydroquinazolin-2-yl)propan-2-ol. Synthesized according to the method of reagent preparation 8 using 2-hydroxy-5,5- dimethylcyclohex-1-enecarboxylate and 2-hydroxy-2-mehylpropanimidainide hydrochloride in step 2. MS(ES) for C₁₃H₁₉ClN₂: 255 (MH⁺).

4-chlro-2,6-dimethyl-5-(1-methylethyl)pyrimidine. Synihesized according to the method of reagent preparation 8 by using 2-iodopropane in step 1. MS (EI) for C₉H₁₃ClN₂: 185 (MH⁺).

(7S)-4-chloro-7-ethyl-2-methyl-5,6,7,8-tetrahydroquinazoline. Synthesized according to the method of reagent preparation 8 by using methyl (4S)-4-ethyl-2- oxocyclohexanecarboxylate (reagent preparation 3) in step 2. MS (EI) for C₁₁H₁₅ClN₂: 211 (MH⁺).

4-Chloro-6,6-dimethyl-2-(2-pyrrolidin-1-ylethyl)-5,6,7,8-tetrahydroquinazoline. Synthesized according to the method of reagent preparation 8 by using 1-pyrrolidinepropanimidamide in step 2. MS (EI) for C₁₆H₂₄ClN₃: 294 (MH⁺).

### Reagent Preparation 9

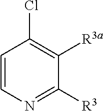

STEP 1: To a solution of phenylmethyl 2-methyl-4-oxo-3,4-dihydropyridine- 1(2H)-carboxylate (J. Biaorg. Med. Chem, 2007, 1106-1116) (2.4 g,.97.8 mmol) in THF (35 mL) was added dropwise a 1M solution of lithium bis(trimethylsilyl)amide in THF (11 mL) at −78° C. The solution was warmed up to 0° C. stirred at this temperature for 1 h, then cooled again to −78° C. 3-Fluorobenzadehyde (1.3 mL, 12.7 mmol) was added in one portion. The reaction was stirred for 4 h while allowing it to slowly warm up to 0° C. Then, saturated ammonium chloride (20 mL) was added, and the layers were separated. The aqueous layer was extracted with ethyl acetate (2×20 mL) and the combined organic layers were washed with saturated sodium chloride (50 mL), dried over sodium sulfate, filtered and concentrated.

Column chromatography on silica (gradient 20 to 100% ethyl acetate in hexanes) afforded phenylmethyl 3-[(3-fluorophenyl)(hydroxy)methyl]-2-methyl-4-oxo-3,4-dihydropyridine- 1(2H)-carboxylate (2.4 g, 66% yield) as mixture of diastereomer, MS (EI) for C₂₁H₂₀FNO₄: 370.1 (MH⁺).

STEP 2: Mesyl chloride (0.31 mL, 3.97 mmol) was added in one portion to a solution of phenylmethyl 3-[(3-fluorophenyl)(hydroxy)methyl]-2-methyl-4-oxo-3,4- dihydropyridine-1(2H)-carboxylate (0.73 g, 1.98 mmol) in anhydrous pyridine (5 mL) a 0° C. The reaction.mixture was -warmed upapropm temperature and stirrcdTor I h. Water (5mL) and ethyl acetate (5 mL)-were added, the layers were separated, and the aqueous layer was extracted with ethyl acetate (3×5 mL). The combined organic layers were washed with saturated sodium chloride (15 mL) dried over sodium, sulfate, filtered and concentrated to afford phenylmethyl 3-{(3-fluorophenyl)[methylsulfonyl)oxy]methyl}-2-methyl-4-oxo-3,4- dihydropyridine-1(2H)-carboxylate. MS (EI) for C₂₂H₂₂FNO₆S: 448.1 (MH⁺).

STEP 3: Phenylmethyl 3-{(3-fluorophenyl)[methylsulfonyl)oxy]methyl }-2- methyl-4-oxo-3,4-dihydropyridine-1(2H)-carboxylate from step 2 was dissolved in THF (30 mL) and potassium tert-butoxide (1.11 g, 9,9 mmol) was added in one portion. After 15 min the reaction mixture was quenched with saturated ammonium chloride (20 mL). The layers were separated and the aqueous layer was extracted with 5:1 chloroform/isopropanol (3×20 mL). The combined organic layers were dried over sodium sulfate, filtered and concentrated. Column chromatography in silica (10% methanol in dichloromethane) afforded 3-[(3- fluorophenyl)methyl)-2-methylpyridin-4(1H)-one (0.230 g, 53% for two steps) ¹H NMR (400 MHz, CDCl₃): 7.30 (d, 1H), 7.18-7.13 (m, 1H), 6.97 (d, 1H), 6.87-6.79 (m, 2H), 6.35 (d, 1H), 3.91 (s, 2H), 2.22 (s, 3H), MS (EI) for C₁₃H₁₂FNO: 218.1 (MH⁺).

STEP 4: A solution of 3-[(3-fluoropheny)methyl))-2-methylpyridin-4(1H)-one (0.07 g, 0.32 mmol) in phosphorous oxychloride (3 mL),was heated to 55° C. for 16 h. Then the solution was cooled to room temperature and concentrated. The remaining residue was dissolved in ethyl acetate (10 mL), washed with 5% sodium bicarbonate (2×5 mL), and saturated sodium chloride (5 mL), dried over sodium sulfate, filtered and concentrated to afford 4-chloro-3-[(3-fluoropheny)methyl]-2-methylpyridine. ¹H NMR (400 MHz, CDCl₃): 8.33 (d, 1H), 7.30-7.23 (m, 2H), 6.92-6.85 (m, 2H)-I), 6.76 (d, 1h), 4.22 (s, 2H), 2.54 (s, 3H). MS (EI) for C₁₃H₁₁CIF: 236.0 (MH⁺).

Using analogous synthetic techniques and substituting with alternative starting reagents in step 1 the following reagents were prepared.

3-benzyl-4-chloro-2-methylpyridine. Synthesized according to the method of reagent preparation 9 using benzaldchyde in step 1. ¹H NMR (400 MHz, CDCl₃): 8.30 (d, 1M), 7.29-7.19 (m, 4H), 7.08 (d. 2H), 4.22 (s, 2H), 2.51 (s, 3H); MS (EI) for C₁₃H₁₂ClN: 218 (MH⁺).

4-chloro-3-(4-fluorobenzyl)-2-methylpyridine. Synthesized, according to the method of reagent of preparation 9 using 4-fluorobenzaldehyde in step 1. ¹H NMR (400 MHz, CDCl₃): 8.32 (d, 1H); 7.29 (d, 1H), 7.05-6.95 (m, 4H), 4.19 (s, 2H), 2.54 (s, 3H): MS (EI) for C₁₃H₁₁ClFN: 236 (MH⁺).

### Reagent Preparation 10

STEP 1: To a solution of ethyl 3-bromobutanoate (6.0 mL, 42 mmol) in N,N- dimethylformamide (20 mL) at 0° C. was added piperidine (8.0 mL, 80 mmol) and the mixture was warmed to room temperature stirred 16 h. The reaction mixture was diluted with ethyl acetate (200 mL) and washed with a solution of brine and 2.0 M aqueous sodium hydroxide (4.1v/v). The organic phase was then dried over anhydrous sodium sulfate, filtered and concentrated to give ethyl 4-piperidin-1-ylbutanoate (6.8 g, 81% yield) as brown oil. MS (EI) for C₁₁H₁₂NO₂: 200 (MH⁺)

Step 2: To a solution of potassium hydroxide (11 g, 0.20 mol) in water (40 mL) was added a solution of ethyl 4-piperidin-1-ylbutanoate (6.8 g, 34 mmol) in ethanol (30 mL) and the mixture was stirred at 35° C. for 2 hours. The reaction was quenched by dropwise addition of 37% aqueous hydrochloric acid (15 mL) and the mixture was concentrated then dried under vacuum. The residue was suspended in chloroform (100 mL) followed by addition of catalytic N,N-dimethylformamide (0.2 mL) then dropwise addition of oxalyl chloride (15 mL, 170 mmol) and the mixture was stirred at 25° C. for 18 hours. The reaction mixture was concentrated to afford crude 4-piperidin-1-ylbutanoyl chloride hydrochloride. To a suspension of the 4-piperin-1-ylbutanoyl chloride hydrochloride (ca. 40 mmol) and 2- methyl-2-thiepseudpurea sulfate (5.6 g, 20 mmol) in acetonitrile (100 mL) was added triethylamine (20 mL, 0.27 mol) in portions while cooling in an ice bath. The reaction was then allowed to warm to 25° C. over 1 h. The reaction mixture was filtered, through Celite with an acetonitrile wash (100 mL). The filtrate was concentrated to afford methyl N,N′-bis-(4- piperidin-1 -ylbutanoyl)imidothiocarbamate (10.6 g, 79% yield) as a brown oil that was used without further purification. MS (EI) for C₂₀H₃₆N₄O₂S: 397 (MH⁺).

Using analogous synthetic techniques and substituting with alternative starting reagents bis-[2-(methoxy)ethoxyl][(methylthio)methylidene]biscarbamate was prepared according to the method of reagent preparation 10 using2-methoxyethyl chloroformate in step 2. MS (EI) far C₁₀H₁₈N₂O₆S: 295 (MH⁺).

Reagent Preparation 11

STEP 1: To a solution of 6-bromo-2-methyl-1H-imidazo[4,5-b]pyridine (3.40 g, 16.0 mmol) and diisopropylethylamine (6.5 mL, 65 mmol) in N,N-dimethyformamide (20 mL) cooled in an ice bath was added dropwise isobuityl chloroformate (2.51 mL, 19.2 mmol) and the mixture was warmed to room temperature. After 1 hour the reaction was diluted with ethyl acetate (80 mL) and washed with water (60 mL), 10% aquepus citric acid (4 mL) and brine (20 mL). The organic phase was dried over anhydrous sodium sulfate, filtered and concentrated to a slurry. The residue was triturated diethyl ether (100 mL) and the solid isolated by filtration to give isobutyl 6-bromo-2-methyl-1H-imidazo[4,5-b ]pyridine-1-carboxytate (2.3 g, 46% yield), MS (EI) for C₁₂H₁₄BrN₃O₂: 313 (MH⁺).

Using analogous synthetic techniques and substituting with alternative starting reagents in step 1 isobutyl 2-(4-bromophenyl)-1H-imidazole-1-carboxylate was prepared according to the method of reagent preparation 11 using 2-(4-bromophenyl)-1-imidazole and isobutyl chloroformate in step 1. MS (EI) for C₁₄H₁₅BrN₂O₂: 324 (MH⁺).

Isobutyl 6-bromo-1H-benzol[d]imidazole-1-carboxylate. Prepared according to the method of reagent preparation 11 using 5-bromo-1H-benzol[d]imidazole in step 1 MS (EI) for C₁₂H₁₃BrN₂O₂: 297/299 (MH⁺).

### Reagent Preparation 12

### 5-Bromo-1-ethyl-1H-benzimidazole

5-bromo-1-ethyl-1H-benzimidazole was prepared in 3 steps from 1,4-dibromo-2- nitrobenzene according to the method described in (Bioorg. and Med. Chem. Leu. 2003, 13, 2485-2488). MS (EI) for C₉H₉BrN₂: 226 (MH⁺).

### Reagent Preparation 13

### N-(5-bromthiazol[5,4-b]pyridin-2-yl)benzamide

STEP 1: To a solution of ammonium thiocyanate (0.4 g, 5.0 mmol) in acetone (5mL) was slowly added benzoyl chloride (0.6 mL, 5.0 mmol) and the suspension was heated to reflux for ten minutes. A solution of 6-bromo-2-chloro-3-pyridinamine (1.0 g, 4.8 mmol) in acetone (10 mL) was then added and the reaction mixture was refluxcd for one hour. After cooling to room temperature the mixture was poured into water and partitioned with ethyl acetate (250 mL). The layers were separated and the aqueous layer was further extracted with ethyl acetate (2x, 100mL). The combined organic layers were washed with brine (2x. 100mL), dried over sodium sulfate, filtered and concentrated until a suspension formed. The white solid was collected by filtration to give N-(6-bromo-2-chloropyridin-3- ylcarbamothioyl)benzainide (1.6g, 89%). ¹H NMR (400 MHz, d₆-DMO): 12.62 (brs, 1H), 12.00 (br s, 1H), 8.37 (d, 1H), 8.00 (2d, 2H), 7.79 (d, 1H), 7.69 (t, 1H), 7.57 (t,2H),MS (EI) for C₁₃H₉BrClN₃OS: 370 (MH⁺).

STEP 2: A solution of N-(64-bromo-2-chloropyridin-3-ylcarbamothioyl)benzamide (1.5 g, 4.0 mmol) and sodium ethoxide (0.54 g, 8.0mmol) in 1-methyl-2-pyrrolidinone (10 mL) was heated to 120° C. for 8 hours. After cooling the reaction mixture to room temperature the mixture was poured into water; The resulting solid was collected by filtration, then washed sequentially with water and diethyl ether. The filter cake was dried to give N-(5-bromothiazol[5,4-b]pyridin-2-yl)benzamide (1.02 g, 76%); ¹H NMR (400MHz, d₆-DMSO): 13.2 (brs, 1H), 8.16-8.10 (m, 3H), 7,72 (d, 1H), 7.70 (t, 1H), 7.59 (t, 2H), MS (EI) for C₁₃H₈Brn₃OS: 336 (MH⁺).

### Reagent Preparation 14

STEP 1: To a solution of 2-amino-5-bromopyridine (5.0 g, 29 mmol) in dioxane (60 mL) was added ethoxycarbonylisothiocyanate (3.4 mL, 29 mmol) in a dropwise manner and the mixture was allowed to stir for 18h at room temperature. The mixiure was then concentrated and the residue triturated with 10% ethyl acetate in hexanes. The solid was collected by filtration and dried to afford ethyl {[(5-bromopyridin- 2yl)amino]carbonothioyl}carbamate (6.2 g, 69%) as a colorless solid. MS (EI) for C₉H₁₀BrN₃O_{2S:} 305 (MH⁺).

STEP 2: {[(5-Bromopyridin-2yl)amino]carbonothioyl}carbamate was converted to 6-bromo-[1,2,4]triazolo[1,5-a]pyridin-2-amine according to methods in the literature, see 1) Monatshefte fuer Chemie. 1983,114(6-7), 789-98 and 2) Synthesis, 2003, 11, 1649-1652. Thus, a mixture of hydroxylamine hydrochloride (375 mg, 5.4 mmol) and DIPEA (560 uL, 3.2 mmol) in 1:1 methanol:ethanol (8 mL) was stirred for 10 minutes at room temperature followed by addition of {[(5bromopyridin-2yl)amino]carbonothioyl}carbamate(500 mg, 1.62 mmol) and the resulting suspension was stirred for 2 h at room temperature then brought to 60° C. for an additional 2 h. The resulting solution was then cooled to room temperature and concentrated. The residue was then partitioned with ethyl acetate and saturated aqueous sodium bicarbonate. The organic solution was washed with brine, dried over anhydrous sodium sulfate then filtered and concentrated to give 6-bromo-[1,2.4]triazolo[1,5-a]pyridin-2- amine (340 mg, 98 % yield) as a colorless crystalline solid. MS (EI) for C₆H₅BrN₄: 214 (MH⁺).

STEP 3: A solution of 6-bromo-[1,2,4]triazolo[1,5-a]pyridin-2-amine (340 mg, 1.6 mmol), di-tert-butyl dicarbonate (370 mg, 1.6 mmol)and catalytic. DMAP was stirred at 35° C. in THF (5 mL) for 18h. An additional equivalent of di-tert-butyl dicarbonate was then added and stirring was continued for 48 h. The solution was then partitioned with ethyl acetate and water. The organic phase was washed with brine, dried over anhydrous sodium sulfate then filtered and concentrated. The residue was taken into dichloromcethane and insoluble starting maierial was removed by filtration. The filirate was concentrated and purified by silica gel chromatography to afford bis-(1,1-dimethylethyl( (6-bromo[1,2,4triazolo[1,5-a]pyridine-2-yl)imidodicarbonate (284 mg, 43% yield) as an off white solid. ¹H NMR (400 MHz, d₆-DMSO): 9.45 (s, 1H), 7.91 (d, 1H), 7.86 (d, 1H), 1.41 (s, 18H).

Using analogous synthetic techniques and substituting with alternative starting reagents bis (1,1-dimethylethyl) (5-bromo-4-methyl-1,3-thiazol-2-yl)imidodicarbonate was prepared according to the method of reagent preparation 14 uUsing 5-bromo-4-methylthiazol- 2-amine in step 3 and conducting the protection step at reflux temperature. ¹H NMR (400 MHz, CDCI₃): 2.30 (s, 3H), 1.53 (s, 18H).

### Reagent Preparation 15

### 6-bromo-1-trityl-1H-imidazo[4,5-b]pyridine and 6-bromo-3-trityl-3H-imidazo[4,5-b]pyridine

STEP 1: A suspension of 2,3-diamino-5-bromopyridine (3.0 g, 16.00 mmol) in formic acid (30 mL) was heated lo reflux for 3 hours. After cooling the reaction mixture to room temperature it was concentrated and the residue was taken into 50% ethyl acetate in toluene (100 mL) then concentrated and the process repeated once more to remove excess formic acid. The resulting solid was triturated with ethyl acetate and the solid residue collected by filtration to give 6-bromo-1H-imidazo[4,5-b]pyridine (3,7 g, 95%). GCMS (EI) for C₆H₄BrN₂: 198 (M⁺).

STEP 2: To a solulion of 6-bromo-1 H-imidazo[4,5-b]pyridine (2.7 g, 11.0 mmol) in dimethylformamide (30 mL) at 0° C. was added 60% sodium hydride in mineral oil (0.53 g, 13.2 mmol) and the reaction mixture was stirred for 30 minutes, followed by the addition of a solution of triphenylmethyl chloride (3.2 g, 11.55 mmol) in diinethylformamide (5 mL). The reaction mixture was stirred at room temperature for 24 hours then quenched by the careful addition of water then partitioned with ethyl acetate (250 mL). The organic phase was washed with 10% aqueous citric acid (2x, 100 mL). brine (100 mL), saturated sodium bicarbonate (100 mL), brine (100 mL) then dried over anhydrous sodium sulfate, filtered and concentrated. Silica gel chromatography (hexane ethyl acetate 9:1 to 4:1) provided 6-bromo- 3-trityl-3H-imidazo[4,5-b]pyridine (1.8 g, 37%). ¹H NMR (400 MHz. CDCI₃): 8.18 (d, 1H), 8.14 (d, 1H), 8.02 (s, 1H), 7.36-7.28 (m, 10H), 7.18-7.14 (m, 5H) and 6-bromo-1-trilyl-1H- imidazo[4,5-b]pyridine (2.9 g, 60%) 1H NMR (400 MHz, CDCI₃): 8.50 (d, 1H), 8.14 (s, 1H), 7.38-7.34 (m, 10H), 7.16-7.12 (m, 5H), 6.84 (d, 1H).

### Reagent Preparation 16

### N-(7-Bromo-[1,2,4]triazolo[1, 5-a]pyridin-2-yl)acetamide

STEP 1: To a solution of 7-Bromo-[1,2,4)triazizolo[1,5-a]pyridin-2-ylamine (prepared using the procedure in WO2006038116) (0.150 g, 0.704 mmol), diisopiopylethylainine (0.363 g, 2.81 mmol), catalytic DMAP (0.09 g, 0.07 mmol) in anhydrous THF (4 mL) was added acetic anhydride (0.216 g, 2.11 mmol). The reaction mixture was stirred al 50° C. for 22 h under (g). After cooling to room temperature the mixture was concentrated, diluted vvidi ethyl acetatef (50 mL), washed with saturated sodium bicarbonate (40 mL), brine (40 mL), and dried over-anhydrous sodium sulfate. Filtration and concentration followed by column chromatography of the residue op silica (95:5 dicholprmethane/methanol) afforded N-(7-bromo-[1,2,4]triazolo[1,5-a]pyridin-2- yl)acetamide (0.170 g, 95% yield) as a brown oil. MS (EI) for C₈H₇BrRN₄O: 256 (MH⁺).

### Reagent Preparation 17 : 1-(4-chloro-6,6-dimethyl-5,6,7,8-tetrahydroquinazolin-2-yl)- N,N-dimethylmethanamine

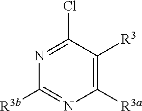

Step 1: To a solution of methyl5,5-dimethyl-2-oxocyclohexanecarboxylate (6.0 g, 33 mmol) and 2-chloroacetimidamide hydrochloride (4.6 g, 36 mmol) in methanol (30 mL) was added sodium methoxide (4.4 M in MeOH, 9.0 mL, 40 mmol). The reaction mixture was stirred al ambient temperature for three hours, and then 1 concentrated. The resulting residue was partitioned between ethyl aceiale and aqueous sodoimin bicarbonate. The organic layer was washed with brine, dried over magnesium sulfate and concentrated. Purification by silica gel chromatography provided 2-(chloromethyl)-6,6-dimethyl-5,6,7,8-tetrahydroquinazolin-4- ol (4.2 g, 57% yield) as a white solid. MS (ES) for C₁₁H₁₅ClN₂O: 227 (MH⁺).

Step 2: To a solution of 2-(chloromethyl)-6,6-dimethyl-5,6,7,38- tetrahydroquinazolin-4-ol (2.5 g; 11 mmol) in THF (10 mL) was added dimethyl amine (2M in THF, 16.5 mL, 33 mmol). The reaction mixture was healed (60° C.) for two hours and then partitioned between ethyl acetate and sodium bicarbonate. The organic layer was washed with brine, dried over magnesium sulfate, filtered and concetrated to provide 2- ((dimethylamino)methyl)-6,6-dimethyl-5,6,7,8,-tetrahydroquinazolin-4-ol, which was used in step 3 without further purification MS (ES) for C₁₃H₂₁N₃O: 236 (MH⁺).

Step 3: To a solution of the final residue from step 2 in CHCl₃ (10 mL) was added POCI₃ (10 mL). The reaction mixture was heated (90° C.) for two hours and concentrated. This residue was partitioned between dichloromethane and aqueous sodium bicarbonate and the resulting organic layer was washed with brine, dried over magnesium sulfate, filtered and concentrated in vacuo. Purification by silica gel chromatography (5-10% concentrated aqueous ammonia in methanol), in chloroform provided 1-(4-chloro-6,6-dimethyl-5,6,7,8- tetrahydroquinazolin-2-yl)-N,N-dimethylmethanamine (1.3 g, 48% yield). ¹H NMR (400 MHz, CD₃OD) δ 4.52 (s, 2H), 3.02 (s, 6H), 2.98 (t, 2H), 2.61 (s, 2H), 1.71 (t, 2H), 1.06 (s, 6H); MS (ES)for C₁₃H₂₀CIN₃: 254 (MH⁺)

Using analogous synthetic techniques and stituting with alternative starting reagents the following compounds of the invention were prepared. Alternative starting materials were obtained, commercially unless otherwise indicated.

(S )-4-chloro-2-((3-fluoropyrrolidin-1 -yl)methyl)-6,6-dimethyl-5,6,7,8- tetrahydroquinazoline. Synthesized according to the meihod of reagent preparation 17 using (S)-3-fluoropyrrolidine in step 2. MS (ES) for C₁₅H₂₁ClFN₃: 298 (MH⁺).

(R)-4-chloro-2-((3-fluoropyrrolidin-yl)methyl)-6,6-dimethyt-5,6,7,8- tetrahydroquinazoline. Synthesized according to the method of reagent preparation 17 using (R)-3-fluoropyrrolidine in step 2. MS (ES) for C₁₅H₂₁ClFN₃: 298 (MH⁺).

4-chloro-2-((3,3-difluoropyrrolidin-1-yl)methyl)-6,6-dimethyl-5,6,7,8- tetrahydroquinazoline. Synthesized according to the method of reagent preparation 17 using 3,3-difluoropyrrolidine in step 2. MS (ES) for C₁₅H₂₀ClF₂N₃: 316 (MH⁺).

N-((4-cbloro-6,6-dimethyl-5,6,7,-tetrahydroquinazolin-2-yl)methyl)-N- methylethanamine. Synthesized according to the method of reagent preparation 17 using N- methylethanamine in step 2. MS (ES) for C₁₄H₂₂CIN₃: 268 (MH⁺).

4-chloro-6,6-dimethyl-2-(piperidin-1-ylmethyl)-5,6,7,8-tetrahydroquinazoline. Synthesized according to the method of reagent preparation 17 using piperidinc in step 2. MS (ES) for C₁₆H₂₄ClN₃: 294 (MH⁺).

N-((4-chloro-6,6-dimethyl-5,6,7,8-terahydroquinazolin-2-yl)methyl)-N- methylpropan-2-amine. Synthesized according to the method of reagent preparation 17 using N-methylpropan-2-amine in step 2. MS (ES) for C₁₅H₂₄CIN₃: 282 (MH⁺).

N-((4-chloro-6,6-dimethyl-5,6,7,8-tetrahydroquinazolin-2-yl)methyl)-N- methylcyclopropanamine. Synthesized according to the method of reagent preparation 17using N-methylcyclopropanamine in step 2l MS (ES) for C₁₅H₂₂CIN₃: 280 (MH⁺).

Benzyl (4-chloro-6,6-dimethyl-5,6,7,8-tetraliydroquinazolin-2- yl)methyt(isopropyl)carbamate. Synthesized according to the method of reagent preparation 17 using propane-2-aminc in step 2 followed by Cbz protection. MS (ES) for C₂₂H₂₈ClN₃O₂: 402 (MH⁺).

4-chloro-6,6-dimethyl-2-(pyrrolidin-1-ylmethyl)-5,6,78,-tetrahydroquinazoline. Synthesized according to the method of reagent preparation 17 using pyrrolidine in step 2. MS (ES) for C₁₅H₂₂ClN₃: 280 (MH⁺).

(S)-1-(4-chloro-7-ethyl-5,6,7,8-tetrahydroquinazolin-2-yl-N,N- dimethylmethanamine. Synthesized according 10 the method of reagent preparation 17 using (S)-methyl 4-ethyl-2-hydroxycyclohex-1-enecarboxylate in step 1. MS (ES) for C₁₃H₂₀ClN₃: 254 (MH⁺).

(4-chloro-5-[(4-fluorophehyl)methyl]-[6-methylpyrimidin-2-yl methyl acetate. Synthesized according to the method of reagent preparation 17 using 2-(chloromethyl)-5-[(4- fluorophenyl)methyl[-6-methy]pyrimidin-4-ol and sodium acetate in acetic acid in step 2. MS (ES) for C₁₅H₁₄ClFN₂O₂: 309 (MH⁺).

4-chloro-2-methoxymethyl)-6,6-dimethyl-5,6,7,8-tetrahydroquinazoline. Synthesized according to the method of reagent preparation 17 using sodium methoxide in step 2. MS (ES) for C₁₂H₁₇ClN₂O: 241 (MH⁺).

Benzyl (4-chloro-6,6-dimethyl-5,6,7,8-tetrahydroquinazolin-2-yl)methyl(ethyl)- carbamate. Prepared according to the method of reagent preparation 17 by using ethylamime in step 2 followed by Cbz protection. MS (EI) for C₂₁H₂₆ClN₃O₂: 388 (MH⁺).

Benzyl (4-chloro-6,6-dimethyl-5,6,7,8-tetrahydroquinazolin-2-yl)methyl(2- fluoroethyl)carbamate. Prepared according to the method of reagent preparation 17 by using fluoroethylamine in step 2 followed by Cbz protection. MS (EI) for C₂₁H₂₅ClFN₃O₂: 406(MH⁺).

N-[(4-chloro6,6-dimethyl-5,6,7,8-tetrahydroquinazolin-2- yl)methyl]cyclopropanamine. Prepared according to the method of reagent preparation 17 by using cyclopropylamine in step 2. MS (EI) for C₁₄H₂₀CIN₃: 266 (MH⁺).

Benzyl (4-chloro-6,6-dimethyl-5,6,7,8-tetrahydroquinazolin-2- yl)methyl(cyclobutyl)carbamate. Prepared according to the method of reagent preparation 17 by using cyclobutylamine in step 2 followed by Cbz protection. MS (Ef) for C₂₃H₂₈ClN₃O₂: 414 (MH⁺).

1-(4-Chloro-5-(cyclopropylmethyl)-6-methylpyrimidin-2-yl)-N,N- dimethyhlmethanamine. Prepared according to the method of reagent preparation 17 by using methyl 2-(cyclopropylmethyl)-3-oxobutanoate (reagent preparation 8) in step 1. MS (EI) for C₁₂H₁₈ClN₃: 240 (MH⁺).

N((4-chloro-6,6-dimethyl-5,6,7,8-tetrahydroquinazolin-2-yl)methyl)-N- ethylethanamine. Prepared according to the method of reagent preparation 17 by using diethylamine in step 2. MS (EI) for C₁₅H₂₄ClN₃: 282 (MH⁺).

4-((4-Chloro-6,6-dimethyl-5,6,7,8-tetrahydroquinazolin-2-yl)methyl)morpholine. Prepared according to the method of reagent preparation 17 by using inorpholihe in step 2. MS (EI) for C₁₅H₂₂ClN₃O: 296 (MH⁺).

N-((4-chloro-6,6-dimethyl-5,6,7,8-tetrahydroquinazolin-2-yl)methyl)-N- ethylpropan-2-amine. Prepared according to the method or reagent preparation 17 by using ethylisopropylamine in step 2. MS (EI) for C₁₆H₂₆ClN₃: 2 (MH⁺).

N-((4-chloro-6,6-dimethyl-5,6,7,8-tetrahydroquinazolin-2-yl)methyl)2- methylpropan-2-amine. Prepared acccording to the method of reagent preparation 17 by using tert-butylamine in step 2. MS (EI) C₁₅H₂₄ClN₃: 282 (MH⁺).

N-((4-chloro-6,6-dimethyl-5,6,7,8-tetrahydroquinazolin-2-yl)methyl)-2- methylpropan-1-amine. Prepared according to the method of reagent preparation 17 by using iso-butylamine in step 2. MS (EI) for C₁₅H₂₄ClN₃: 282 (MH⁺).

Benzyl (4-chloro-6,6-dimethyl-5,6,7,8-tetrahydroquinazolin-2-yl)methyl(2,2- difluoroethyl)carhamate. Prepared according to the method of reagent preparation 17 by using 2,2-difluoroethylamine in step 2 followed by Cbz protection. MS (EI) for C₂₁H₂₄ClF₂N₃O₂: 424 (MH⁺).

N-((4-chloro-6,6-dimethyl-5,6,7,8-tetrahydroquinazolin-2-yl)methyl)-2.2.2- trifluoroethanamine. Prepared according t the method of reagent preparation 17 by using 2,2,2-trifluoroethylamine in step 2. MS (EI) for C₁₃H₁₇CIF₃N₃: 308 (MH⁺).

N-((4-chloro-6,6-dimethyl-5,6,7,8-tetrahydroquinazolin-2-yl)methyl)-1- cyclopropylethanamine. Prepared according to the method of reagent preparation 17 by using 1-cyclopropylethanamine in step 2. MS (EI) for C₁₆H₂₄CIN₃; 294 (MH⁺).

(4-Chloro-6,6-dimethyl-5,67,8-tetrahydroquinazolin-2-yl)methyl acetate. Prepared according to the method of reagent preparation 17 by using potassium acetate in step 2.MS (EI) for C₁₃H₁₇ClN₂O₂: 269 (MH⁺).

Benzyl (4-chloro-6,6-dimethyl-5,67,8-tetrahydroquinazolin-2- yl)methyl (eyclopentyl)carbamate. Prepared according to tee method of reagent preparation 17 by using cyclopentylamine in step 2 followed by Cbz protection. MS (EI) for C₂₄H₃₀ClN₃O₂: 428 (MH⁺).

Ethyl2-((4-chloro-6,6-dimethyl-5,6,7,8-tetrahydroquinazolin-2- yl)methylamino)propanoate. Prepared accordingao the method of reagent preparation 17 by using alanine ethyl ester in step 2. MS (EI) for C₁₆H₂₄ClN₃O₂: 326 (MH⁺).

1-(4-Chloro-5,6-dimethylpyrimidin-2-yl)-N,N-dimethylmethanamine. Prepared according to the method of reagent preparation 17 by using methyl 2-methyl-3-oxobutanoate in step 1 in step 2. MS (EI) for C₉H₁₄CIN₃: 200 (MH⁺).

1-(4-chloro-5-(4-fluorobenzyl)-6-methylpyrimidin-2-yl)-N,N- dimethylmethanamine. Synthesized according to the method of reagent preparation 17 using methyl 2-(4-fluorobenzyl)-3-oxobutanoate in step 1 . ¹H NMR (400 MHz, CDCl₃): 7.08-7.05 (m, 2H), 7.00-6.96 (m, 2H), 4.14 (s, 2H), 3.68 (s, 2H), 2.51 (s, 3H), 2.38 (s, 6H).

1-(4-chloro-5-isopropyl-6-methylpyrimidin-2-yl)-N,N-dimethylmethanamine. Synthesized according to the method of reagent preparation 17 using methyl 2-acetyl-3- methylbutanoate in step 1. MS (EI) for C₁₁H₁₈N₃Cl: 228, 230-(MH⁺, CI isotope pattern).

(S)-benzyl sec-butyl((4chloro-6,6-dimethyl-5,6,7,8tetrahydroquinazoline-2- yl)methyl) carbamate Synthesized according to the method of reagent preparation 17 using (S)-butan-2-amine in step 2 followed Cbz-protection prior to step 3. MS (ES) for C₂₃H₃₀ClN₂: 416 (MH⁺).

(R)-benzyl sec-benyl((4-chloro-6,6-dimethyl-5,6,7,8-tetrahydroquinazolin-2- yl)methyl)carbaniate Synthesized according to the method of reagent preparation 17 using (R)-butan-2-amine in step 2 followed Cbz-protcction prior lo step 3. MS (ES) for C₂₃H₃₀ClN₃O₂: 416 (MH⁺).

1-(4-chloror6-ethyl-5-methylpyrimidin-2-yl)-N,N-dimethylmethanamine Synthesized according to the method of reagent preparation 17using methyl 2-methyl-3- oxopentanoate in step 1. MS (ES) for C₁₀H₁₆ClN₃: 214 (MH⁺).

1 -(4-chloro-5-isopropylpyrimidin-2-yl)-N,N-dimethylmethanamine Synthesized according to the method of reagent preparation 17 using methyl 2-methyl-3-oxopentanoate (Elaridi et al. Tetrahedron: Asymmetry 2005, 16(7) 1309-1319) in step 1.

N-((4-chloro-6,6-dimethyl-5,6,7,8-tetrahydroquinazolin-2-yl)methyl)-N-methyl-2- nitrobenzenesulfonamide Synthesized according to the method of reagent preparation 17using methylamine in step 2 followed by protection as the 2-nitrobenzencsuulfonamide prior to step 3. ¹H NMR (400 MHz, CDCI₃) ε 8.18-8.13 (m, 1H), 7.71-7.62 (m, 2H), 7.61-7.57 (m, 1H), 4.69 (s, 2H), 3.08 (d, 3H), 2.73 (t, 2H)., 2.47 (s, 2H), 1.60 (t, 2H), 1.01 (s, 6H); MS (ES) for C₁₈H₂₁CIN₄O₄S: 425 (MH⁺).

N-((4-cldoio-6,6-dimethyl-5,6,7,8-tetrahydroquinazolin-2- yl)methyl)methanesulfonamide Synthesized according to the method of reagent preparation 17 using atnmpnia in step 2 followed by mesylation prior to step 3. ¹H NMR (400 MHz, CDCl₃) δ 4.49 (d, 2H), 3.01 (s, 3H), 2.90 (t, 2H), 2.54 (s, 2H), 1.67 (t, 2H), 1.05 (s, 6H): MS (ES) for C₁₂H₁₈CIN₃O₂S: 304 (MH⁺).

1-(4-chloro-5-ethyl-6-methylpyrimidin-2-yl)-N,N-dimethylmethanamine. Synthesized according to the method of reagent preparation 17 using ethyl 2-ethyl-3- oxobutanoate in step . ¹H NMR (400 MHz, CDCI₃) δ 3.64 (s, 2H), 2.78 (q. 2H) 2.58 (s, 3H), 2.36(s, 6H), 1.19 (t, 3H); MS (ES) for C₁₀H₁₆ClN₃: 214 (MH⁺).

4-chloro-6,6-dimethyl-2-({[2-(methyloxy)ethyl]oxy}methyl)-5,6,7,8- tetrahydroquinazoline. Synthesized according to the method of reagent preparation 17 using sodium hydride and 2-methoxyethanol in N,N-dimethylformamide) in step 2: MS (ES) for C₁₄H₂₁CIN₂: 285(MH⁺).

N-[4(4-chloro-6,6-dimethyl-5,6,7,8-tetrahydroquinazolin-2-yl)methyl]-2- (methyloxy)ethanamine. Synthesized according to the method of reagent preparation 17 using 2-methoxyethanamine in step 2. MS (ES) for C₁₄H₂₂ClN₃O: 284 (MH⁺).

N-((4-chloro-5-(4-florobenzylyl)-6-methylpyrimidin-2- yl)methyl)cyclopropanamine. Prepared according to the method of reagent preparation 17 by using methyl 2-(4-fluorobenzyl)-3-oxobutanoate in step 1and cyclopropylamine in step 2. MS (EI) for C₁₆H₁₇CIFN₃: 306 (MH⁺).

1-(4-chloro-7-methoxy-6,6dimethyl-5,6,7,8-tetrahydroquinazolin-2-yl)-N,N- dimethylmethanamine. Prepared according to the method of reagent preparation 17 using methyl 5,5-dimethyl-2-oxocyclohex-3T-enecarboxylate (Can. J. Chem., 1981. 59, 601-608) in step 1. MS (ES) for C₁₄H₂₂ClN₃O: 284 (MH⁺).

### Reagent Preparation 18: Phenylmethyl (2R)-2-(4-chloro-6,6-dimethyl-5,6,7,8- tetrahydroquinazolin-2-yl)pyrrolidine-1-carboxylate

STEP 1: To sodium mmethoxide (30wt % in methanol, 8 mg, 0.05 mmol) was added a solution of (R)-benzyl 2-cyanopyrrolidine- 1-carboxylate (189 mg, 0.82 mmol) in methanol (1 mL) at room temperature and the reaction mixture was stirred for one hour. Ammoniun chloride (44 mg, 0.82 mmol) was introduced and the stirring was continued for an additional two hours, followed by the addition of methyl 5,5-dimethyl-2-oxocyclohexanecarboxylate (100 mg, 0.54 mmol) and sodium methoxide (30wt % in methanol. 293 mg, 1.63 mmol). The stirring was continued for two more hours. The reaction mixture was quenched with water (10 mL), neutralized with 1 N hydrohloric acid and extracted with ethyl acetate (3x 10 mL). The combined extract was washed with water (20 mL) and brine, dried over sodium sulfate, filtered, concentrated and purified by gradient flash chromatography (25% to 95% ethyl acetate in hexane) to give phenylmethyl (27R)-2-(4-hydroxy-6,6-dimethyl-5,6,7,8- tetrahydroquinazolin-2-yl)pyrrolidine-1-carboxylate (186 mg, 90%). MS (EI) for C₂₂H₂₇N₃O₃: 3.81 (MH⁺).

STEP 2: A mixture phenylmethy (2R)-2-(4-hydroxy-6,6-dimethyl-5,6,7,8- tetrahydroquinazolin-2-yl)pyrrolidine-1-carboxylate (150 mg, 0.39 mmol), and phosphorous oxychloride (1 mL) in chloroform (3 mL) was stirred at 80° C. for one hour. After cooling to room temperature the reaction mixture was concentrated and the residue was partitioned between saturated sodium bicarbonate (20 mL) and ethyl acetate (20 mL). The mixture was stirred for 15 minutes and pH was maintained above 7 by the addition of solid sodium bicarbonate. The organic layer was separated and washed with water (10 mL) and brine, dried over sodium sulfate, filtered and concentrated to give phenylmethyl (2R)-2-(4-chIoro-6,6- dimethyl-5,6,7,8-tetrahydroquinazolin-2-yl)pyrrolidine-1-carboxylate (1.17 mg, 74%). MS (EI) for C₂₂H₂₆ClN₃O₂: 400 (MH⁺).

Using analogous synthetic techniques and substituting with alternative starting materials in step 1 the following reagents of the invention were prepared. Alternative starting materials were obtained commercially unless otherwise indicated.

Phenylmethyl (2S)-2-(4-chloro-6,6-dimethyl-5,6,7,8tetrahydroquinazolin-2- yl)pyrrolidine-1-carboxylate. Prepared according to the method of reagent preparation 18by using (S)-benzyl 2-cyanopyrrolidine-1-carboxylate in step 1 (118 mg, 75%). MS (EI) for C₂₂H₂₆ClN₃O₂: 400 (MH⁺).

Phenylmethyl 2-(4-chloro-6,6-dimethyl-5,6,7,8-tetrahydroquinazolin-2- yl)pyrrolidine-1-carboxylate. Prepared according to the method of reagent preparation 18 by using (R.S)-benzyl 2-cyanopyrrolidine-1-carboxylate in step 1 (118 mg, 75%). MS (EI) for C₂₂H₂₆ClN₃O₂: 400 (MH⁺).

### Reagent Preparation 19: Phenylmethyl {[6-bromo-3-({[2- (trimethylsilyl)ethyl]}methyl)-3H-imidazo[4,5-b]pyridine-2-yl]methyl}methylcarbamate

STEP 1: To a mixture of 2-[(benzyloxycarbonyl)(meyhyl)amino]acetic acid (0.42 g, 1.88 mmol), 0-(7-azabenzotriazol-1-yl)-N,N,N′,N′-tetramethyluronium hexafluorophosphate (0.75 g, 1.97 mmol) in N,N-dimethylformamide (3.0 mL), N,N-diisopropylethylamine (0.72 mL, 4.12 mmol) was added and the reaction mixture was stirred for 30 minutes at room temperature, followed by the addition of 5-bromo-2,3- diaminopyridine (0.35 g, 1.86 mmol), then stirred for 16 hours. It was diluted with ethyl acetate (50 mL), washed with aqueous lithium chloride (2×20 mL) and brine, dried over sodium sulfate, filtered and concentrated. Gradient flash chromatography (35% to 85% ethyl acetate in hexane) provided phenylmethyl {2-[(2-amino-5-bromopyridin-3-yl)amino]-2- oxoethyl}methylcarbamate (0.70 g, 96%), MS (EI) for C₁₆H₁₇BrN₄O₃: 394 (MH⁺).

STEP 2: A solution of phenylmethyl {2-[(2-amino-5-bromopyridin-3-yl)amino]- 2-oxoethyl}methylcarbamate (0.30 g, 0.76 mmol) in acetic acid (7.5 mL) was heated in a microwave apparatus (250 W) for 30 min. at 120° C. After cooling it to room temperature the reaction mixture was concentrated and the pH was adjusted to 8 by the addition of saturated aqueous sodium bicarbonate. The precipitating soild was collected by filtration, washed with water and dried in vacuo to give phenylmeihyl [(6-bromo-1H-imidazo[4,5-b]pyridin-2- yl)methyl]methylcarbamate (0.22 g, 76%), MS (EI) for C₁₆H₁₅BrN₄O₂: 376 (MH⁺).

STEP 3: To a solution of phehylmelhyl [(6-bromo)-1 H-imidazo[4,5-b]pyridin-2- yl)methyl]methylcarbamate (0.22 g, 0.59 mmol) in N,N-dimethylformamide (3.0 mL) was added 60% sodium hydride in mineral oil (56 mg, 1.48 mmol) and the reaction mixture was stirred for 30 minutes at room temperature, followed by the addition of 2- (trimethylsilyl)ethoxymethyl chloride (0.11 mL, 0.62 mmol). The reaction mixture was stirred at room temperature for 16 hours then it was quenehed by the careful addition of saturated aqueous ammonium chloride and partitioned with ethyl acetate (20 mL) and water (20 mL). The organic layer was separated and washed with 10% aqueous citric acid (2 x 20 mL) and brine (20 mL), dried over sodium sulfate, filtered and concentrated. Gradient flash chromatography (15% to 35% ethyl acetate in hexane) gave phenylmethyl {[6-bromo-3-({[[2- (trimethylsily)ethyl]oxy}methyl)-3H-imidazo[4,5-b]pyridin-2-yl]methyl]methylcarbamate (0.28 g, 93%). MS (EI) for C₂₂H₂₉BrN₄O₂Si: 506 (MH⁺).

Using analogous synthetic techniques and substituting with alternative starting materials and reagents in step 1 or step 2 and step 3 the following reagents of the invention were prepared. Alternative stalling materials were obtained commercially unless otherwise indicated.

Phenylmethyl {(1R)-1-[6-bromo-3-({[2-(trimethylsilyl)ethyl]oxy}methyl)-3H- imidazo[4,5-b]pyridin-2-yl]ethyl]({[2-(trimethylsilyl)ethyl]oxy}methyl)carbamate. Synthesized according to the method of reagent preparation 19 by using 5-bromo-2,3- diaminopyridine and N-(benzyloxycarbonyl)-D-alanine in stop 1 and 2- (trimethylsilyl)ethoxymethyl chloride in step 3. MS (EI) for C₂₈H₄₃BrN₃₄O₄Si₂: 636. (MH⁺).

Phenylmethyl {(1S)-1-[6-bromo-3-([[2-(trimethylsilyl)ethyl]oxy]methyl)-3H- imidazo[4,5-b]pyridin-2-yl]ethyl]([[2-(trimethylsilyl)ethy]oxy]methyl)carbamate. Synthesized according to the method of reagent preparation 19 by usinig 5-bromo-2,3- diaminopyridine and N-(benzyloxycarbonyl)-L-alanine in step 1 and 2- (trimethylsilyl)ethoxymelhyl chloride in step 3. MS (EI) for C₂₈H₄₃BrN₃₄O₄Si₂: 636 (MH⁺).

7-Bromo-2-methyl-3-({[2-(methyloxy)ethyl]oxy}tmethyl)-3H-imidazo[4,5- c]pyridine and 7-bromo-2-methyl-1-({[2-(methyloxy)ethyl]oxy}methyl)-1H-imidazo[4,5- c]pyridine. Synthesized according to the method of reagent preparation 19 by using 5- bromopyridine-3,4-diamine and triethyl orthoacetate in step 2 and methoxyethoxymethyl chloride in step 3. ¹H NMR (400 MHz, CDCI₃): 8.83 (s, 2H), 8.44 (s, 2H), 5.88 (s, 2H), 5.66 (s, 2H) 3.36 (s, 3H), 3.37 (s, 3H), 2.98 (s, 4H), 2.91 (s, 4H), 2.73 (s, 3H), 2.75 (s. 3H): MS (EI) for C₁₁H₁₄BrN₃O₂: 301 (MH⁺).

1-(6-Bromo-3H-imidazo[4,5-b]pyridin-2-yl)ethanol. Synthesized according to the method of reagent preparation 19 by using D,L-lactic acid in step 1. MS (EI) for C₈H₈BrN₃O: 241 (MH−).

Tert-butyl 6-bromo-2-(difluoromethyl)-1H-benzo[d]imidazole-1-carboxylate. Synthesized according to the method of reagent preparation 19 using 4-bromobenzene-1,2- diamine and difluoroacetic acid in step 1 and BOC protection with di-tert-butyl dicarbonate in step 3. MS (EI) for 6-bromo-2-(difluoromethyl)-1H-benzo[d]imidazole (step 2) C₈H₅BrF₂N₂: 247, 249 (MH⁺, Br isotope pattern).

1,1-Dimethylethyl 6-bromo-2,4-dimethyl-1H-benzimidazole-1-carboxylate. Synthesized according to the method of reagent preparation 19 using 5-bromo-3- methylbenzene-1,2-diamine and acetylation using acetyl chloride in tetrahydrpfuran in step 1 the BOC protection with di-tert-butyl dicarbonate in step 3. MS (EI) for C₁₄H₁₇BrN₂O₂: 267. 269 (M-Boc, Br isotope pattern).

1.1-Dimethylethyl 5-bromo-6-fluoro-2-methyl-1H-benzimidazole-1-carboxylate. Synthesized according to the method of reagent preparation 19 using 4-bromo-5- fluorobenzene-l,2-diamine and triethyl orthoacetate in step 2 and BOC protection with di-tert-butyl dicarbonate in step 3. MS (EI) for C₁₃H₁₄BrFN₂O₂: 271, 273 (M-Boc, Br isotope pattern).

2-Methylpropyl 5-bromo-4-fluoro-2-methyl-1H-benzimidazole-1-carboxylate. Synthesized according to the method of reagent preparation 19 using 5 4-bromo-3- fluorobenzene-1,2-diamine and acetylation with acetic anhydride in tetrahydrofurane n step 1 then treatment with isobutyl chloroformate in step 3. MS (EI) for C₁₃H₁₄BrN₂O₂: 328, 330 (MH⁺, Br isotope pattern).

6-Bromo-2-ethyl-3-({[2-(trimethylsilyl)ethyl]oxy-}methyl)-3H-imidazo[4,5- [d]pyridine. Synthesized according to the method of reagent preparation 19 by using 5-bromo- 2,3-diaminopyridine and trimethyl orthopropionate in step 2 and 2- (trimethylsilyl)ethoxymeihyl chloride in step 3. MS (EI) for C₁₄H₂₂BrN₃OSi: 357 (MH⁺).

2-Methylpropyl 6-bromo-2-cyclopropyl-3H-imidazo[4,5-b]-3- carboxylate. Synthesized according to the method of reagent preparation 19 by using 5- bromo-2,3-diaminopyridine and acylation with cyclopropylcarbonyl chloride in step 1 and treatment with isobutyl chloroformate in step 3. MS (EI) for C₁₄H₁₆BrN₃O₂: 339 (MH⁺).

2-Methylpropyl 5-bromo-2-(fluoromethyl)-1H-benzimidazole-1-carboxylate. Synthesized according to the method of reagent preparation 19 using 4-bromobenzene-1,2- diamine and fluoroacetic acid in Step 1 then treatment with isobutyl chloroformate in step 3. MS (EI) for C₁₃H₁₄BrFN₂O₂: 330(MH⁺).

### Reagent Preparation 20

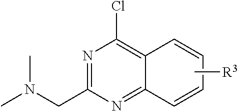

STEP 1: To a solution of 4-methoxyanthranilic acid (5.0 g, 30.0 mmol) in a mixture of 10% methanol in tetrahydrofuran (100 mL) was added dropwise (trimethylsilyl)diazomethane (2.0 M solution in diethyl ether, 18.0 mL, 36.0 mmol) at 0° C. The reaction mixture was stirred for 16 hours at room temperature then quenched by the addition of glacial acetic acid (0.1 mL). The reaction mixture was concentrated and the residue was partitioned between saturated sodium bicarbonate (50 mL) and ethyl acetate (250 mL). The organic layer was separated and washed with water (50 mL), saturated sodium bicarbonate (50 mL) and brine (50 mL), dried over sodium sulfate, filtered and concentrated to give methyl 2-amino-4-methoxybenzoate as an oil (5.4 g, quantitative), MS (EI) for C₉H₁₁NO₃: 182(MH⁺).

STEP 2: To a mixture of methyl 2-amino-4-methoxybenzoate (5.4 g, 30.0 mmol) and chloroacetonitrile (2.8 mL, 45.0 mmol) was added anhydrous hydrogen chloride (4M solution in 1,4-dioxane, 20.0 mL, 80 mmol) and the reaction mixture was stirred at 50° C. for 30 minutes. After cooling it to room, temperature the resulting slurry was diluted with diethyl ether (100 mL) and the stirring was continued for an additional 30 minutes. The off-white precipitate was collected by filtration, washed with diethyl ether and dried in vacuo to provide 2-(chloromethyl)-7-(methyloxy)quinazolin-4-ol hydrochloride (7.5 g, 96%). MS (EI) for C₁₀H₉ClN₂O₂: 225 (MH⁺).

STEP 3: To a solution of dimethylamine (2M solution in tetrahydrofuran, 40.0 mL, 80.0 mmol) was added 2-(chloromethyl)-7-(methyloxy)quinazolin-4-ol hydrochloride (7.5 g. 29 mmol) and the reaction mixture was stirred for 90 minutes at 50° C. After cooling it to room temperature the reaction mixture was concentrated and the residue was partitioned between water (100 mL) and ethyl acetate (250 mL). The organic layer was separated and washed with water (100 mL), saturated sodium bicarbonate (100 mL) and brine (100 mL), dried over sodium sulfate, filtered and concentrated to give 2-[(dimethylamino)methyl]-7- (methyloxy)quinazolin-4-ol (6.6 g, 97%). MS (EI) for C₁₂H₁₅N₃O₂: 234 (MH⁺).

STEP 4: A solution of 2-[(dimethylamino)methyl]-7-(methyloxy)quinazolin-4-ol (6.6 g, 28.0 mmol) in a mixuire of chloroform (15.0 mL) and phosphorous oxychloride (15.0 mL) was heated to reflux for 90 minutes. After cooling it to room temperature the reaction mixture was concentrated and the residue was partitioned between saturated sodium bicarbonate (100 mL) and ethyl acetate (400 mL) and the mixture was stirred for 30 minutes. The organic layer was separated and washed with saturated sodium bicarbonate (2x 100 mL) and brine (200 mL), dried,over sodium sulfate, filtered and concentrated. Purification by silica gel column chromatography using 15% methanol containing 0.5% triethylamine in ethyl acetate provided 1-[4-chloro-7-(methyloxy)quinazolin-2-yl]-N,N-dimethylmethanamine (7.0 g, quantitative). MS (EI) for C₁₂H₁₄ClN₃O: 252 (MH⁺).

Using analogous synthetic techniques and substituting with alternative starting materials in step 2 the following reagents of the invention were prepared. Alternative starlting materials were obtained commercially unless otherwise indicated.

1-(4-chloro-6-fluoroquinazolin-2-yl)-N,N-dimethylmethanamine. Prepared according to the method of reagent preparation 20 by using methyl 2-amino-5-fluorobenzoate in step 2. MS (EI) for C₁₁H_{ClFN}₃: 240 (MH⁺).

### Reagent Preparation 21:5-Bromo-1-methyl-1H-pyrrolo[2,3-b]pyridine

STEP 1: To a mixture of 5-bromo-1H-pyrrolo[2,3-b]pyridine (207 mg. 1.05 mmol), sodium hydride (29 mg, 1.21 mmol) in tetrahydrofuran (5mL) was added iodomethane (164 mg, 1.15 mol) then stirred for 2 h at room temperature. The reaction mixture was carefully quenched with water then extracted with ethyl acetate (3x). The combined organic layers were dried over magnesium sulfate, filtered and concentrated under reduced pressure. The crude product was purified by silica gel chromatography to give 5- bromo-1-methyl-1H-pyrrolo[2,3-b]pyridine. MS (EI)for C₈H₇BrN₂: 209, 211 (MH⁺, Br pattern).

Using analogous synthetic techniques and substituting with alternative starting reagents in step 1 the following reagent was prepared. 5-bromo-1-ethyl-1H-pyrrolo[2.3- b]pyridine. Synthesized according to the method of reagent preparation 21 using iodoethane. MS (EI) for C₉H₉BrN₂: 223, 225 (MH⁺, Br pattern).

### Reagent Preparation 22: (4-(4-Bromopbenyl)-1H-imidazol-2yl)methanol

STEP 1: To a solution of ethyl thiooxamate (10.0 g, 75 mmol) in dichloromethane (400 mL) was slowly added trimethyloxonium tetrafluoroborate (13.1 g, 89 mmol) at 0° C. After 10 min the ice bath was removed. and the reaction mixture was stirred overnight.The solvent was removed to afford ethyl 2-imino-2-(methylthio)acetate (12.0g. 66.6%) as tetrafluoroborate salt which was used without further purification.

STEP 2: A mixture of 2-amino-4-bromoacetophenone hydrochloride (4.0 g, 16.0 mmol), sodium acetate (6 lg, 90.0 mmol), acetic acid (4.6 mL, 80.0 mmol) and ethyl 2- imino-2-(methylthio)acetate (7.7g, 32.0 mmol) in dioxane (40 mL) was stirred at 95° C. overnight. The reaction mixture was carefully neutralized with saturated NaHC03 solution and extracted with ethyl acetate. The organic solution was dried over sodium sulfate and concentrated. Purification by silica gel column chroinatography (ethyl acetate:hexanes 1:1) afforded ethyl 4-(4-bromophenyl)-1H-imidazole-2-carboxylate (3.53 g, 75.0%). MS (EI) for C₁₂H₁₁BrN₂O₂: 296 (MH⁺).

STEP 3: To a solution of ethyl 4-(4-bromophenyl)-1H-imidazole-2-carboxylate (1.30 g, 4.40 mmol) in THF (30 mL) was slowly added Red-AI ( 65 wt % in toluene, 2.0 mL, 6.16 mmol) at −25° C. The recatopm mixture was stirred for 4 h at the same temperature then slowly warmed to 0° C. over 1 h and quenched with 20 % sodium tartrate solution (30 mL). The reaction was extracted with ethyl acetate (70 mL) and the organic layer was left for 3h at room temperature. A solid separated and was collected by filtration, washed with ethyl acetate and dried to afford (4-(4-bromophenyl)-1H-imidazol-2yl)methanol (778 mg, 71.0 %). MS (EI) for C₁₀H₉BrN₂O: 254.1 (MH⁺).

### Reagent Preparation 25

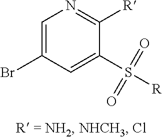

STEP 1: To a solution of (R)-pyrrolidin-3-ol (32 mg, 0.37 mmol) and potassium carbonate (102 mg, 0.74 mmol) in dioxane (2 mL) and water (400 uL) was added 2-amino-5- bromopyridine-3-sulfonyl chloride (100 mg, 0.37 mmol, prepared according to the methods in WO2008144463). The reaction mixture was stirred for 2 h at rt. Saturated sodium bicarbonate was then added, and the aqueous solution was extracted twice with ethyl acetate. The combined organic extracts were dried over magnesium sulfate, filtered, and concentrated in vacuo to provide (R)-1-(2-amino-5bromopyridin-3-ylsulfonyl)pyrrolidin-3-ol (87.3 mg, 0.27 mmol, 73% yield) as a white solid. ¹H NMR (400 MHz, DMSO₆-d6) δ 8.31 (d, 1H), 7.92 (d, 1H), 6.85 (br s, 2H), 5.02 (br s, 1H), 4.23 (dt, 1H), 3.38-3.25 (m, 3H), 3.14-3.06 (m, 1H), 1.92-1.81 (m, 1H), 1.77-1.67 (m, 1H); MS (EI) for C₉H₁₂BrN₃O₃S: 322, 324 (Br isotopes, MH⁺).

Using analogous synthetic techniques and substituting with alternative starting reagents in step 1 the following reagents were prepared. Alternative starting inaterialswere obtained commercially unless otherwise indicated.

2-amino-5-bromo-N-(2-methoxyethyl)pyridine-3-sulfonamide. Prepared according to the methods described in reagent preparation 25 using 2-methoxyethanamine in step 1.

2-amino-5-bromo-N-(2,2,2-trifluoroethyl)pyridine-3-sulfonamide. Prepared according to the methods described in reagent preparation 25 using 2,2,2-trifluoroethanamine in step 1.

2-amino-5-bromo-N-(2-hydroxyethyl)-N-methylpyridine-3-sulfonamide. Prepared according to the methods described in reagent preparation 25 using 2-(methylamino)ethanol in step 1.

2-amino-5-bromo-N-(2-hydroxypropyl)pyridine-3-sulfonamide. Prepared according to the methods described in reagent preparation 25 using, 1-aminopropan-2-ol in step 1. MS (EI) for C₈H₁₂BrN₃O₃S: 310 , 312 (Br isotopes. MH⁺).

2-amino-N-(azetidin-3-yl)-5-bromopyridine-3-sulfonamide. Prepared according to the methods described in reagent preparation 25 using tert-butyl 3-aminoazetidine-l- carboxylate in step 1.

2-amino-5-bromo-N-(2,3-dihydroxypropyl)pyridine-3-sulfonamide. Prepared according to the methods described in reagent preparation 25 using 3-aminopropane-1,2-diol in step 1. MS (EI) for C₈H₁₂BrN₃O₄S: 326, 328 (Br isotopes. MH⁺).

1-(2-amino-5-bromopyridin-3-ylsulfonyl)piperidin-3-ol. Prepared according to the methods described in reagent preparation 25 using pipcfidin-3-ol in step 1. MS (EI) for C₁₀H₁₄BrN₃O₃S: 336, 338 (Br Isotopes. MH⁺).

2-amino-N-(3-amino)-2,2-dimethylpropyl)-5-bromopyridine-3-sulfonamide. Prepared according to the methods described in reagent preparation 25 using 2,2- dimethylpropane-1,3-diamine in step 1. MS (EI) for C₁₀H₁₇BrN₄O₂S: 337, 339 (Br isotopes, MH⁺).

2-amino-5-bromo-N-(3-hydroxy-2,2-dimethylpropyl)pyridine-3-sulfonamide. Prepared according to the methods described in reagent preparation 25 using 3-amino-2,2T dimethylpropan-1-ol in step 1. MS (EI) for C₁₀H₁₆BrN₃O₃S: 338, 340 (Br isptopes. MH⁺).

2-amino-5-bromo-N-(1-hydroxy-2-methylpropan-2-yl)pyridine-3-sulfonamide. Prepared according to the methods described in reagent preparation 25 using 2-amino-2- methylpropan-ol in step 1. MS (EI) for C₉H₁₄BrN₃O₃S: 324 326 (Br isotopes, MH⁺).

terl-butyl 4-((2-amino)-5-bromopyridine-3-sulfonamido)methyl)piperidine-1- carboxylate. Prepared according to the methods described in reagent preparation 25 using tert-butyl 4-(aminomethyl)piperidine-1-ccarboxylate in step 1. MS (EI) for C₁₆H₂₅BrN₄O₄S: 393, 395 (Br isotopes, MH⁺t-butyl).

2-amino-5-bromo-N-((1-methylpiperidin-4-yl)methyl)pyridine-3-sulfonamide. Prepared according to the methods described in reagent preparation 25 using (1- methylpiperidin-4-yl)methanamine in step 1. MS (EI) for C₁₂H₁₉BrN₄O₂S: 363, 365 (Br isotopes, MH⁺).

tert-butyl 1-((2-amino-5-bromopyridine-3- sulfonamido)mcthyr)cyclopropyrcarbamate. Prepared according to the methods described in reagent preparation 25 using tert-butyl 1-(aminomethyl)cyclopropylcarbamate in step 1. MS (EI) for C₁₄H₂₁BrN₄O₄S: 365, 367 (Br isotopes, MH⁺t-butyl).

tert-butyl trans-4-(2-anmio-5-bromopyridine-3-sulfonamido)cyclohexylcarbamate. Prepared according to the methods described in reagent preparation 25 using tert-butyl trans-4-aminocyclohexylcarbamate in step 1.

benzyl 1-(2-amino-5-bromopyridine-3-sulfonamido)propan-2-ylcarbamate. Prepared according to the method described in reagent preparation 25 using benzyl 1-aminopropan-2-ylcarbamate in step 1.

2-amino-5-bromo-ethylpyridine-3-sulfonamide. Prepared according to the methods described in reagent preparation 25 using ethylamine in step 1. ¹H NMR (400 MHz, CDCl₃) δ 8.28 (d, 1H), 8.07 (d, 1H), 5.63 (brs, 2H), 4.61 (t, 1H), 3.06-2.97 (m, 2H), 1.14 (t, 3H); MS (EI) for C₇H₁₀BrN₃O₂S: 280, 282 (Brisotopes, MH⁺).

2-amino-5-bromo-N-isopropylpyridine-3-sulfonamide. Prepared according to the methods described in reagent preparation 25 using isopropylamine in step 1. ¹H NMR (400 MHz, CDCl₃) δ 8.28 (d, 1H) 8.09 (d, 1H), 5.59 (hr s, 2H), 4.52 (d, 1H), 3.50-3.39 (m, 1H). 1.11 (d, 6H): MS (EI) for C₈H₁₂BrN₃O₂S: 294, 296 (Br isotopes, MH⁺).

2-amino-5-bromo-N-(2-(dimethylamino))ethyl)pyridine-3-sulfonamide. Prepared according to the methods described in reagent preparation 25 using N,N-dimethylethane-1,2- diamine in step 1.¹H NMR (400 MHz, CDC₃) δ 8.27 (d, 1H), 8.08 (d, 1H), 5.66 (br s, 2H), 2.99-2.93 (m, 2H), 2.36-2.30 (m, 2H), 2.12 (s, 6H); MS (EI) for C₉H₁₅BrN₄O₂S: 323, 325 (Br isotopes, MH⁺).

2-amino-5-bronmo)-N-(2-hydroxyethyl)pyridine-3-sulfonamide. Prepared according to the methods described in reagent preparation 25 using 2-aminoethanol in step 1. ¹H NMR (400 MHz, CDCI₃) δ 8.29 (d, 1 H), 8.08 (d, 1H), 5.65 (br s, 3H), 5.23 (br s, 1H), 3.76-3.67 (m, 3H), 3.16-3.07 (m, 3H); MS (EI) for C₇H₁₀BrN₃O₅S: 296, 298 (Br isotopes, MH⁺).

1-(2-amino-5-bromopyridin-3-ylsulfonyl)-3-(hydroxymethyl)azetidin-3-ol. Prepared according to the methods described in reagent preparation 25 using 3-(hydroxymethyl)azetidin-3-ol (prepared according to procedures described in WO2007044515) in step 1. ¹H NMR (400 MHz, CD₃OD) δ 8.28 (d, 1H), 8.00 (d, 1H), 3.90-3.84 (m, 2H), 3.70-3.64 (m, 2H), 3.32-3.29 (m, 2H): MS (EI) for C₉H₁₂BrN₃O₄S: 338, 340 (Br isotopes, MH⁺).

2-(2-amino-5-bromopyridine-3-sulfonamido)acetaniide. Prepared according to the methods described in reagent preparation 25 using 2-aminoacetamide hydrochloridein step 1. ¹H NMR (400 MHz, DMSO-d₆) δ 8.26 (d, 1H), 8.18 (br s, 1H), 7.90 (d, 1H), 7.34 (brs, 1H), 7.12 (br s, 1H), 6.84 (br s, 2H), 3.45 (s, 2H); MS:(EI) for C₇H₉BrN₄O₃S: 309, 311 (Br isotopes, MH⁺).

tert-butyl 3-(2-amino-5-bromnopyridine-3-sulfonamido)-2- hydroxypropylcarbamate. Prepared according to the methods described in reagent preparation 25 using tert-butyl 3-amino-2-hydroxypropylcarbamate in step 1. ¹H NMR (400 MHz, DMSO-d₆) δ8.26 (d, 1H), 7.88 (d, 1H), 6.82 (bis, 2H), 6.74 (t, 1H), 5.02 (d, 1H), 3.50-3.42 (m, 1H), 2.88 (t, 2H), 2.82 (dd, 1H), 2.57 (dd, 1H), 1.37 (s, 9H); MS (EI) for C₁₃H₂₁BrN₄O₅S: 369, 371 (Br isotopes, MH⁺t-Bu).

5-bromo-3-(3-(dimethylamino)azetidin-1-ylsulfonyl)pyridin-2-amine. Prepared according to the methods described in reagent preparation 25 using N,N-dimethylazetidin-3- amine hydrochloride in step 1. ¹H NMR (400 MHz, DMSO-d₆) δ 8.39 (d, 1H), 7.92 (d, 1H), 6.90 (br s, 2H), 3.88-3.76 (m, 2H), 3.63-3.54 (m, 2H), 3.07-2.97 (m, 1H), 1.96 (s, 6H): MS (EI) for C₁₀H₁₅BrN₄O₂S: 335, 337 (Br isotopes, MH⁺).

5-bromo-N-2-hydroxyethyl)-2-(methylamino)pyridine-3-sulfonamide. Prepared according to the methods described in reagent preparation 25 using 5-bromo-2- (methylamino)pyridine-3-sulfonyl chloride (prepared from 5-bromo-N-methylpyridin-2- amine using analogous conditions to those described in WO2008144463) and 2-aminoethanol in step 1. ¹H NMR (400 MHz, CDCl₃) δ 8.28 (d, 1H), 8.00 (d, 1H), 7.10-7.03 (m, 1H), 6.48- 6.39 (m, 1H), 3.93 (t, 1H), 3.60 (q, 2H), 3.04-2.96 (m, 5H); MS (EI) for C₈H₁₂BrN₃O₃S: 310, 312 (Br isotopes, MH⁺).

N-(1-(2-amino-5-bromopyridin-3-ylsulfonyl)azetidin-3-yl)-N-methyl-2- nitrobenzenesulfonamide. Prepared according to the methods described in reagent preparation 25 using N-(azetidin-3-yl)-N-methyl-2-nitrobenzenesulfonamide in step 1. ¹H NMR (400 MHz, CDCl₃) δ 8.32 (d, 1H),8.06-8.03 (m, 1H), 8.00 (d, 1H), 7.77-7.72 (m, 2H), 7.70-7.65 (m, 1H), 5.78 (brs. 2H), 4.90-4.80 (m, 2H), 4.01 (dd, 2H), 4.01 (dd, 2H), 2.91 (s, 3H): MS (EI) for C₁₅H₁₆BrN₅O₆S: 506, 508 (Br isotopes, MH⁺).

tert-butyl 4-(2-amino-5-bromopyridin-3-ylsuIfonyl)piperazine-1-carboxylate. Prepared according to the methods described in reagent preparation 25 using tert-butyl piperazine-1-carboxylate in step 1. ¹H NMR (400 MHz, DMSO-d₆) δ 8.34 (d, 1H), 7.86 (d, 1H), 6.90 (br s, 2H), 3.4-3.35 (m, 4H), 3.09-3.02 (m, 4H), 1.37 (s, 9H): MS (EI) for C₁₄H₂₁BrN₄O₄S: 367, 365 (Br isotopes, MH⁺-t-Bu).

3-(3-amino-3-methylazetin-1-ylsulfonyl)-5-bromopyrind-2-amine. Prepared according to the methods described in reagent preparation 25 using 3-methylazelidin-3-amine hydrochloride (prepared by procedures described in WO2007007057 followed by benzylidene deprotection) in step 1. ¹H NMR (400 MHz. DMSO-6) δ 8.37 (d, 1H), 7.88 (d, 1H), 6.86 (br s, 2H), 3.58-3.47 (m, 4H), 2.06 (br s, 2H), 1.22 (s, 3H); MS (EI) for C₉H₁₃BrN₄O₂S: 321, 323 (Br isotopes, MH⁺).

tert-butyl 2-(2-amino-5-bromopyridine-3-sulfonamido)-2-methylpropylcarbamate. Prepared according 10 the methods described in reagent preparation 25 using tert-butyl 2- amino-2-methylpropylearbamate in step 1. ¹H NMR (400 MHz, CDCI₃) δ 8.26 (d, 1H), 8.08 (d, 1H), 5.89 (br s, 1H), 5.60 (br s, 2H), 5.04 (t, 1H), 3.12 (d, 2H), 1.46 (s, 9H), 1.19 (s, 6H); MS (EI) for C₁₄H₂₃BrN₄O₄S: 367, 369 (Br isotopes, MH⁺-t-Bu).

tert-butyl 5-((2-amino-5-bromopyridine-3- sulfonamido)methyl)hexahydrocyclopenta[c]pyrrole-2(1H)-carboxylate. Prepared according to the methods described in reagent preparation 25 using tert-butyl 5-(aminomethyl)hexahydrocyclopenta[c]pyrrole-2(1H)-carboxylate (prepared from substrates described in WO2004006846) in step 1. ¹H NMR (400 MHz, CDCl₃) δ 8.28 (d, 1H), 8.06 (d, 1H), 5.65 (br s, 2H), 5.03 (t, 1H), 3.41 (br s, 2H), 3.17 (br s, 2H), 2.93 (t, 2H), 2.63-2.54 (m, 2H), 2.14-1.98 (m, 3H), 1.46 (s, 9H), 1.09-0.98 (m, 2H); MS (EI)for C₁₈H₂₇BrN₄O₄S: 419, 421 (Br isotopes, MH⁺-t-Bu).

tert-butyl 1-(2-amno-5-bromopyridine-3-sulfonamido)butan-2-ylcarbamate. Prepared according lo the methods described in reagent preparation 25 using tcrt-butyl 1- aminobutan-2-ylcarbamate in step . ¹H NMR (400 MHz, DMSO-d6) δ 8.28 (d, 1H), 7.89 (d, 1H), 6.78 (br s, 2H), 6.57 (d, 1H), 3.33-3.26 (m, 1H), 2.77-2.65 (m, 2H), 1.53-1.30 (m, 1H), 1.37 (s, 9H), 1.28-1.15 (m, 1H), 0.76 (t, 3H): MS (EI) for C₁₄H₂₃BrN₄O₄S: 367, 369 (Br isotopes, MH⁺-t-Bu).

tert-butyl 4-(2-amino-5-bromopyridine-3-sulfonamido)-2-methylbutan-2- ylcarbamate. Prepared according to the methods described in reagent preparation 25 using tert-butyl 4-amino-2-methylbutan-2-ylcarbamale in step 1. ¹H NMR (400 MHz, CDCI₃) δ 8.27 (d, 1H), 8.06 (d, 1H), 5.64 (br s, 2H), 5.07 (br s, 1H), 4.41 (br s, 1H), 2.98 (q, 2H), 1.93- 1.85 (m, 2H), 1.41 (s, 9H), 1.22 (s, 6H): MS (EI) for C₁₅H₂₅BrN₄O₄S: 381, 383 (Br isotopes, MH⁺-t-Bu).

b 2-amino-N-(2-amino-2-methylpropyl)-5-bromopyridine-3-sulfonamide. Prepared according to the methods described in reagent preparation 25 using 2-methylpropane-1,2- diamine in step 1. ¹H NMR (400 MHz, CDCl₃) δ 8.27 (d, 1H), 8.07 (d, 1H), 5.69 (br s. 2H), 2.73 (s, 2H), 1.12 (s, 6H); MS (EI) for C₉H₁₅BrN₄O₂S: 323, 325 (Br isotopes, MH⁺).

tert-butyl 1-(2-amino-5-bromopyridin-3-ylsulfonyl)azetidin-3-ylcarbamate. Prepared according to the methods described in reagent preparation 25 using tert-butyl azetidin-3-ylcarbamate in step 1. ¹H NMR (400 MHz, CDCI₃) δ 8.31 (d, 1H), 8.00 (d, 1H), 5.76 (br s, 2H), 4.80 (br s, 1H), 4.50-4.3.6 (m, 1H), 4.11 (t, 2H), 3.75 (t, 2H), 1.42 (s, 9H); MS (EI) for C₁₃H₁₉BrN₄O₄S: 407, 409 (Br isotopes, MH⁺).

tert-butyl 1-(2-amino-5-bromopyridin-3-ylsulfonyl)piperidin-4-ylcarbamate sulfonamide. Prepared according to the methods described in reagent preparation 25 using tert-butyl piperidin-4-ylcarbamate in step 1.

2-amino-5-bromo-N-(2-hydroxy-2-methlpropyl)pyridine-3-sulfonamide. Prepared according to the methods described in reagenl preparation 25 using 1-amino-2- methylpropan-2-ol instep 1.

2-Amino-5-bromo-N,N-dimethylpyridine-3-sulfonamide. Prepared according to the method of reagent preparation 25 by using dimethylamine in step 1. MS (EI) for C₇H₁₀BrN₃O₂S: 280 (MH⁺).

5-Bromo-3-(morpholinosulfonyl)pyridin-2-amine. Prepared according to the method of reagent preparation 25 by using morpholine in step 1, MS (EI) for C₉H₁₂BrN₃O₃S: 322 (MH⁺);

5-Bromo-3-(4-methylpipazin-1-ylsulfonyl)pyridin-2-amine. Prepared according to the method of reagent preparation 25 by using N-methylpiperazine in step 1. MS (EI) for C₁₀H₁₅BrN₄O₂S: 335 (MH⁺).

3-(Azetidin-1-ylsulfonyl)-5-bromopyridin-2-amine. Prepared according to the method of reagent preparation 25 by using N-methylpiperazine in step 1. MS (EI) for C₈H₁₀BrN₃O₂S: 292 (MH⁺).

2-Amino-5-bromo-N-methylpyridine-3-sulfonamide. Prepared according to the method of reagent preparation 25 by using methylamine in step 1. MS (EI) for C₆H₈BrN₃O₂S: 266 (MH⁺).

1 -(2-Amino-5-bromopyridin-3-ylsulfonyl)azetidin-3-ol. Preparcd according to the method of reagent preparation 25 by using azetidinol in step 1. MS (EI) for C₈H₁₀BrN₃O₃S: 308 (MH+).

5-Bromo-3-(pyrrolidin-1 -ylsulfonyl)pyridin-2-amine. Prepared according to the method of reagent preparation 25 by using pyrrolidine in step 1. MS (EI) for C₉H₁₂BrN₃O₂S: 306 (MH⁺).

1-(2-Amino-5-bromopyridin-3-ylsulfonyl)pyrrolidin-3-ol. Prepared according to the method of reagent preparation 25 by using 3-pyrrolidinol in step 1. MS: (EI) for C₉H₁₂BrN₃O₃S: 322 (MH⁺).

2-Amino-5-bromo-N-cyclobutylpyridine-3-sulfonamide. Prepared according to the method of reagent preparation 25 by using cyclobutylamine in step 1. MS (EI) for C₉H₁₂BrN₃O₂: 306(MH⁺).

2-Amino-5-bromopyridine-3-sulfonamide. Prepared according to the method of reagent preparation 25 by using ammoniumhydroxide in step 1. MS (EI) for C₅H₆BrN₃O₂S: 252 (MH⁺).

2-Amino-5-bromo-N-ethyl-N-methylpyridine-3-sulfonamide. Prepared according to the method of reagcnl preparation 25 by using N-methylethylamine in step 1. MS (EI) for C₈H₁₂BrN₃O₂S: 294 (MH⁺).

5-Bromo-3-(3,3-difluoroazetidin-1-ylsulfonyl)pyridinr-2-amine. Prepared according to the method of reagent preparation 25by using 3,3-difluoroazetidine in step 1. MS (EI) for C₈H₈BrF₂N₃O₂S: 328 (MH⁺).

2-Amino-5-bromo-N-(1-hydroxypropan-2-yl)pyridine-3-sulfonamide. Prepared according to the method of reagent preparation 25 by using 2-aminopropan-1-ol in step 1. MS (EI):Tor C₈H₁₂BrN₃O₃S: 3.10 (MH⁺).

2-Amino-5-bromo-N-(2-fluorethyl)pyridine-3-sulfonamide. Prepared according to the method of reagent preparation 25 by using 2-fluoroethylamine in step 1. MS (EI) for C₇H₉BrFN₃O₂S: 298 (MH⁺).

tert-Butyl 1-(2-amino-5-bromopyridin-3-ylsulfonyl)pyrrolidin-3-ylcarbamate. Prepared according to the method of reagent preparation 25 by using tert-butyl pyrrolidin-3- ylcarbamate in step 1. MS (EI) for C₁₄H₂₁BrN₄O₄S: 365 (MH³⁰ ).

1-(2-Amino-5-bromopyridin-3-ylsulfonyl)piperidin-4-ol. Prepared according to the method of reagent preparation 25 by using 4-hydroxypiperidine in step 1. MS (EI) for

C₁₀H₁₄BrN₃O₃S: 336(MH⁺)

tert-Butyl 1-(2-amino-5-bromopyridin-3-ylsulfonyl)piperidin-3-ylcarbamate. Prepared according to the method of reagent preparation 25 by using tert-butyl piperidin-3- ylcarbamate in step 1. MS (EI) for C₁₅H₂₃BrN₄O₄S: 379 (MH⁺-tBu).

tert-Butyl 2-(2-amino-5-bromopyridine-3-sulfonamido)ethylcarbamate. Prepared accordingto the method of reagent preparation 25 by using tert-butyl 2-aminoethylcarbamate in step 1. MS (EI) for C₁₂H₁₉BrN₄O₄S: 339 (MH⁺-tBu).

2-Amino-5-bromo-N-(3-hydroxypropyl)pyridine-3-sulfonamide. Prepared according to the method of reagent preparation 25 by using 3-hydroxypropylaniine in step 1. MS (EI) for C₈H₁₂BrN₃O₃S: 310 (MH⁺).

tert-Butyl 3-(2-amino-5-bromopyridine-3-sulfonamido)propylcarbamate. Prepared according to the method of reagent preparation 25 by using tert-butyl 2-aminopropylcarbamate in step 1 . MS (EI) for C_H₂₁BrN₄O₄S: 353 (MH⁺-tBu).

2-Amino-5-bromo-N-(3,3,3-trifluoro-2-hydroxypropyl)pyridine-3-sulfonamide. Prepared according to the method of reagent preparation 25 by using 3-amino-1,1,1- trifluoropropan-2-ol in step 1. MS (EI) for C₈H₉BrF₃N₃O₃S: 364 (MH⁺).

tert-Butyl 5-(2-amino-5-bromopyridin-3-ylsulfonyl)hexahydropyrrolo[3,4- c]pyrrole-2(1H)-carboxylate. Prepared according to the method of reagent preparation 25 by using tert-butyl hexahydropyrrolo[3,4-c]pyrrole-2(1 H)-carboxylate in step 1. MS (EI) for C₁₆H₂₃BrN₄O₄S: 391 (MH⁺-tBu)

tert-Butyl 1-(2-amino-5-bromopyridin-3-ylsulfonyl)-3-methylpyrrolidin-3- ylcarbamate. Prepared according to the method of reagent preparation 25 by using tert-butyl 3-methylpyrrolidin-3-ylcarbamate in step 1. MS (EI) for C₁₅H₂₃BrN₄O₄S: 379 (MH⁺-tBu).

(1S,4S)-tert-Butyl 5-(2-amino-5-bromopyridin-3-ylsulfonyl)-2,5- diazabicyclo[2.2]heptane-2-carboxylate. Prepared according to the method of reagent preparation 25 by using (1S, 4S)-tert-buty, 2,5-diazabicyclo[2.2.1]heptane-2-carboxylate in step 1. MS (EI) for C₁₅H₂₁BrN₄O₄S: 377 (MH⁺-tBu).

(R)-tert-Butyl 2-((2-amino-5-bromopyridine-3-sulfonamido)methy))pyrrolidine-1- carboxylate. Prepared according to the method of reagent preparation 25 by using, (R)-tert- butyl 2-(aminomethyl)pyrrolidine-1-carboxylate in step 1. MS (EI) for C₁₅H₂₃BrN₄O₄S: 335 (MH⁺-Boc).

(S)-tert-Butyl 2-((2-amino-5-bromopyridine-3-sulfonamido)pyrrolidine-1- carboxylate. Prepared according to the method of reagent preparation 25 by using (S)-tert- butyl 2-(aminomethyl)pyrrolidine-1-carboxylate in step 1. MS (EI) for C₁₅H₂₃BrN₄O₄S: 335 (MH⁺-Boc).

(1R, 4R)-tert-Butyl 5-(2-amino-5-bromopyridin-3-ylsulfonyl)-2,5- diazabicyclo[2.2]hepiane-2-carboxylate. Prepared according to the method of reagent preparation 25 by using (1R,4R)-tert-butyl 2,5-diazabicyclo[2.2.1]heptane-2-carboxylate in step 1. MS (EI) for C₁₅H₂₁BrN₄O₄S: 377 (M⁺-Boc).

tert-Butyl 4-(2-amino-5-bromopyridine-3-sulfonamido)piperdine-1-carboxylate. Prepared according to the method of reagent preparation 25 by using tert-butyl 4- aminopiperidine-1-carboxylate in step 1. MS (EI) for C₁₅H₂₃BrN₄O₄S: 379 (MH⁺-Boc).

5-Bromo-3-((1S,4S)-5methyl-2,5-diazabicyclo[2,2,1]heptan-2- ylsulfohyl)pyridin-2-amine. Prepared according to the method of reagent preparation 25 by using (1S,4S)-2-methyl-2,5-diazabicyclo[2.2,1 ]heptarie in step 1, MS (EI) for C₁₁H₁₅BrN₄O₂S: 347 (MH⁺).

(S)-tert-Butyl 1-(2-amino-5-bromopyridin-3-ylsulfonyl)pyrrolidin-3-ylcarbamate. Prepared according to the method of reagent preparation 25 by using (S)-tert-butyl pyrrolidin- 3-ylcarbamate in step 1. MS (EI) for C₁₄H₂₁BrN₄O₄S: 421 (MH⁺).

(R)-tert-Butyl 1-(2-amino-5-bromopyridin-3-ylsulfonyl)pyrrolidin-3-ylcarbamate. Prepared according to the method of reagent preparation 25 by using (R)-tert-butyl pyrrolidin-ylcarbamate in step 1. MS (EI) for C₁₄H₂₁BrN₄O₄S; 421 .(MH⁺).

tert-Butyl 8-(2-amino-5-bromopyridin-3-ylsulfonyl)-8-azabicylo[3,2.1]octan-3- ylcarbamate. Prepared accordingto the method of reagent preparation 25 by using tcrt-butyl 8-azabicyclo[3.2.1]octan-3-ylcarbarbamate (WO 2009055077) in step 1. MS (EI) for C₁₇H₂₅BrN₄O₄S: 461 (MH⁺).

2,2,2-Trichloroethyl 3-(2-amino-5-bromopyridine-3-sulfonamido)-8- azabicyco[3,2,1]octane-8-carboxylate. Prepared according to the method of reagent preparation 25 by using 2,2,2-trichloroethyl 3-amino-8-azabicyclo[3.2.1 ]octane-8- carboxylate (WO 2009055077) in step 1. MS (EI) for C₁₅H₁₈BrCl₃N₄O₄S: 535 (MH⁺).

(R)-tert-Butyl 3-((2-amino-5-bromopyridine-3-sulfonamido)methyl)pyrrolidine-1- carboxylate. Prepared according to the method of reagent preparation 25 by using (S)-tert- butyl 3-(aminomethyl)pyrrolidine-1-carboxylale instep 1. MS (EI)for C₁₅H₂₃BrN₄O₄S: 435(MH⁺).

(S)-tert-Butyl 3-((2-amino-5-bromopyridine-3-sulfonamido)methyl)pyrrolidine-1- carboxylate. Prepared according to the method of reagent preparation 25 by using (R)-tert- butyl 3-(aminomethyl)pyrrolidine-1-carboxylate in step 1. MS (EI) for C₁₅H₂₃BrN₄O₄S: 435 (MM⁺).

(R)-tert-Butyl 3-(2-amino-5-bromopyridine-3-sulfonamido)pyrrolidine-1- carboxylate. Prepared according to the method of reagent preparation 25 by using (R)-tert- butyl 3-aminopyrrolidine-1-carboxylate in step 1. MS (EI) for C₁₄H₂₁BrN₄O₄S: 421 (MH⁺).

(S)-tert-Butyl 3-(2-amino-5-bromopyridine-3-sulfonamido)pyrrolidine-1- carboxylate. Prepared according to the method of reagent preparation 25 by using (S)-tert- butyl 3-aminopyrrolidine-1-carboxylate in step 1. MS(EI) for C₁₄H₂₁BrN₄O₄S: 421 (MH⁺).

tert-Butyl 3-((2-amino-5-bromopyridine-3-sulfonamido)methyl)piperdine-1- carboxylate. Prepared according to the method of reagent preparation 25 by using tert-butyl 3-(aminomethyl)piperidine-1-carboxylate in step 1. MS (EI) for C₁₆H₂₅BrN₄O₄S: 449 (MH⁺).

tert-Butyl 2-((2-amino-5-bromopyridine-3-sulfonamido)methyl)piperidine-1- carboxylate. Prepared according to the method of reagent preparation 25 by using tert-butyl 2-(aminomethyl)piperidine-1-carboxylate in step 1. MS (EI) for C¹⁶H₂₅₆BrN₄O₄S: 449 (MH⁺).

(R)-tert-Butyl 3-((2-amino-5-bromopyridine-3-sulfonamido)methyl)piperidihe-1- carboxylate. Prepared according to the method of reagent preparation 25 by using (S)-tert- butyl 3-(aminomethyl)piperidine-1-carboxylate in step 1. MS (EI) for C₁₆H₂₅BrN₄O₄S: 449 (MH⁺).

(S)-tert-Butyl 3-((2-amino-5-bromopyridine-3-sulfonamido)methyl)piperidine-1- carboxylatc. Prepared according to the method of reagent preparation 25 by using (R)-lcrt- butyl 3-(aminomethyl)piperidine-1-carboxylate in step 1. MS (EI) for C₁₆H₂₅BrN₄O₄S: 449(MH⁺).

S)-2-amino-5-bromo-N-((1-methylpiperdine-3-yl)methylpyridine-3-sulfonamide. Prepared according to the method of reagent preparation 25 by using (R)-(1- methylpiperidin- 3-yl)methanamine in step 1. MS (EI) for C₁₂H₁₉BrN₄O₂S: 363 (MH⁺).

2-amino-5-bromo-N-[(3R)-1-methylpyrrolidin-3-yl]pyridine-3-sulfonamide. Synthesized according to the method of reagent preparation 25 by using (R)-1- methylpyrrolidin-3-amine hydrochloride (synthesized according to the method of Journal of Medicinal Chemistry (2002)., 45(3), 721-739) in step 1. MS (EI) for C₁₀H₁₅BrN₄O₂S: 334 336 (MM⁺, Br isotope pattern).

2-amino-5-bromo-N-{[(3S)-1-methylpyyrrolidin-3-yl]methyl]pyridine-3- sulfonamide. Synthesized according to the method of reagent preparation 25 by using (R)-(1- methylpyrrolidin-3-yl)methanamine hydrobromide (synthesized according to the methods of WO 2006028904 for the synthesis of benzyl [[(R)-1-(tert-butoxycarbonyl)pyyrrolidin-3- yl]methyl (carbamate, WO 2006002047 for yhe synthesis of (S)-benzyl pyrrolidin-3- ylmethylcarbamate and Journal of Medicinal Chemistry (2002) 45(3), 721-739 for the synthesis of (R)-benzyl (1-methylpyrroiidin-3-yl)methylcarbamate, using (R)-3- (aminomethyl)-1-(tert-butyloxycarbonyl)pyrrolidine as starting material) in step 1. MS (EI) for C₁₁H₁₇BrN₄O₂S: 348, 350 (MH⁺, Br isotope pattern).

tert-Butyl 6-(2-amino-5-bromopyridin-3-ylsulfonyl)-2,6-diazaspiro[3,3]heptane-2- carboxylate. Prepared according to the method of reagent preparation 25 by using tert-butyl 2,6-diazaspiro[3,3]heptane-2-carboxylate in step 1. MS (EI) for C₁₅H₂₁BrN₄O₄S: 377 (MH⁺-tBu).

(S)-tert-Bulyl 1-(5-bromo-2-chloropyridin-3-ylsulfonyl)pyrrolidin-3-ylcarbamate. Prepared accordingg, to the methods idescribed in reagent preparation 25 using 5-bromo-2- chloropyridine-3-sulfonyl chloride and (S)-tert-butyl pyrrolidin-3-ylcarbamate in step 1. ¹H NMR (400 MHz, CDC₃) δ 8.61 (d, 1H), 8.52 (d, 1H), 4,67 (s, 1H), 4.25 (s, 1H), 3.57 (m, 4H), 3.34 (m, 1H), 2.22 (m, 1H), 1.92 (m, 1H), 1.45 (s, 9H), MS (ES) for C₁₄H₁₉BrClN₃O₄S: 440, 442 (Br isotopes, MH⁺).

tert-Butyl 3-((2-amino-5-bromopyridine-3-sulfonamido)methyl)azetidine-1 - carboxylate. Prepared according to the methods described in reagent preparation 25 using tert-butyl 3-(aminomethyl)azetidine-1-carboxylate instep 1. MS (ES) for C₁₄H₂₁BrN₄S: 421, 423 (Br isolopes. MH⁺).

### Reagent Preparation 26: N-(5-bromo-2-methylpyridin-3-yl)methanesulfonamide

STEP 1: A solution of 5-bromo-2-methylpyridin-3-amine (187 mg,3 1.0 mmol) and diisopropylethylamine (523 uL, 3.0 mmol) in dichloromethane (5 mL) was cooled to 0° C., and then methanesulfonyl chloride (155uL, 2.0 mmol) was added slowly. The reaction mixture was stirred at 0° C. for 8 min and was then warmed to rt. After stirring for 1 h, the volatile materials were removed in vacuo. The residue was then dissolved in methanol (2,5 mL) and aqueous sodium hydroxide (2 M, 1.5 mL, 3mmol)was added. The reaction mixture was stirred for 1 h 40 min at rt. Water was then added to the mixture which was subsequently extracted twice with dichloromcthane. The combined organic extracts were extracted with aqueous citric acid (10%). The organic phase was discarded, and the aqueous phase was basified to pH˜7.5 with aqueous sodium hydroxide (1 M). The aqueous mixture was extracted three times with dichloromethane. The combined organic extracts were dried over magnesium sulfate, filtered, and concentrated in vacuo.The residue was purified by flash chromatography (50% hexanes : 50% ethyl acetate) to provide N-(5-bromo-2-methylpyridin- 3-yl)methanesulfonamide (111 mg, 0.42 mmol, 42% yield) as a white solid. ¹H NMR (400 MHz, DMSO-d6) δ 9.58 (s, 1H), 8.44 (d, 1H), 7!87 (d, 1H), 3.10 (s, 3H), 2.47 (s, 311); MS (EI) for C₇H₉BrN₂O₂S: 265, 267 (Br isotopes, MH⁺).

Using analogous synthetic techniques and substituting with alternative starting reagents in step 1 the following reagents were prepared. Alternative starting materials were obtained commercially unless otherwise indicated.

N-(5-Bromo-2-chlorophenyl)methanesulfonamide. Prepared according to the methods described in reagent preparation 26 using 5-bromo-2-chloroaniline in step 1. ¹H NMR (400 MHz, CDCl3) δ 7.83 (d, 1H), 7.32-7.23 (m, 2H), 6.80 (br s. 1H), 3.06 (s, 3H); MS (EI) for C₇H₇BrClNO₂S: 282, 284, 286 (Br+CI isotopes, MH⁺).

N-(5-Bromo-2-methoxypyridin-3-yl)methanesulfonamidc. Prepared according to the methods described in reagent preparation 26 using 5-bromo-2-methoxypyridin-3-amine in step 1. ¹H NMR (400 MHz, CDCl₃) δ 7.97 (d, 1H), 7.90 (d, 1H), 6.73 (br s, 1H), 4.00 (s, 3H), 3.05 (s, 3H); MS (EI) for C₇H₉BrN₂O₃S: 281, 283 (Br isotopes, MH⁺).

N-(5-Bromo -2-cyanopyrlin-3-yl)methanesulfonamide. Prepared according to the methods described in reagent preparation 26 using 3-amino-5-bromopicolinonitrile in step 1. ¹H NMR (400 MHz, CDCI₃) δ 8.55 (d, 1H), 8.29 (d, 1H), 7.00 (br s, 1H), 3.21 (s,3 3H); MS (EI) for C₇H₆BrN₃O₂S: 276, 278 (Br isotopes, MH⁺).

N-(5-Bromopyridin-3-yl)methanesulfonamidc. Prepared according to the methods described in reagent preparation 26 using 5-bromopyridin-3-amine in step 1. MS (EI) for C₆H₇BrN₂O₂S: 251, 253 (Br isotopes, MH⁺).

N-(5-Bromo-2-chloropyridin-3-yl)-2-chloro-6-methylbenzcnesulfonamide. Prepared according to the methods described in reagent preparation 26 using 5-bromo-2- chloropyridin-3-amineand 2-chloro-6-methylbenzene-1-sulfouyl chloride in step 1. MS (EI) for C₁₂H_{BrCl}₂N₂O₂S: 393, 395, 397 (Br+CI isotopes, MH⁺).

N-(5-Bromo-2-fluoropyridin-3-yl)methanesulfonamide. Prepared according to the methods described in reagent preparation 26 using 5-bromo-2-fluoropyridin-3-am3ine in step 1. MS (EI) for C₆H₆BrFN₂O₂S: 269, 271 (Br isotopes, MH⁺).

N-(5-Bromo-2-chloropyridin-3-yl)acetamide. Prepared according to the methods described in reagent preparation 26 using 5-bromo-2-chlorbpyridin-3-amine and acetyl chloride in step 1.

Methyl 5-bromo-2-chloropyridin-3-ylcarbamate. Prepared according to the methods described in reagenl preparation 26 using 5-bromo-2-chloropyridin-3-amine and methyl chloroformate in step 1

### Reagent Preparation 27: 5-bromo-2-chloro-3-(methylsulfonylmethl)pyridine

STEP 1.5: A mixture of 5-bromo-2-chloro-3-(chlorohmethyl)pyridine (124 mg, 0.52 mmol) and sodium methanesulfinate (52 mg, 0.52 mmol) in dioxane (1.4 mL) and water (1.4 mL) was heated to 110° C. in a microwave reactor for 15 min. After cooling to rt, water was added to the reaction mixture which was subsequently extracted twice with ethyl acetate. The combined organic extracts were dried oyer magnesium sulfate, filtered, and concentrated in vacuo to provide 5-bromo-2-chloro-3-(methylsulfonylmethyl)pyridine (140 mg, .049 mmol. 94% yield) as a yellow solid. ¹H NMR (400 MHz, DMSO-d6) δ 8.63 (d,1H), 8.21 (d, 1H), 4.70 (s, 2H), 3.10 (s, 3H), MS (EI) for C₇H₂BrClNO₂S: 284, 286, 288 (Br+Cl isotopes, MH⁺).

Using analogous synthetic techniques and substituting with alternative starting reagents in step 1 the following reagent was prepared. Alternative starting materials were obtained commercially unless otherwise indicated.

5-Bromo-3-(methylSulfonylmethyl)pyridin-2-amine. Prepared according to the methods described in reagenl preparation 27 using 5.-bromo-3-(bromomethyl)pyridin-2-amine hydrochloride in step 1. ¹H NMR (400 MHz, DMSO-d6) δ 8.03 (d, 1H), 7.59 (d, 1H), 6.35 (brs, 2H), 4.44 (s, 2H), 2.95 (s, 3H); MS (EI) for C₇H₇BrN₂O₂S: 265, 267 (Br isotopes, MH⁺).

### Reagent Preparation 28: N-(5-bromo-2-chloropyridin-3-yl)-N-methylmethanesulfonamide

STEP 1: A solution of N-(5-bromo-2-chloroppyridin-3-yl)methanesulfonamide (96 mg, 0.34 mmol, reagent preparation 24) in DMF (1 mL) was treated with potassium carbonate (93 mg, 0.68 mmol) and iodomethane (33 uL, 0.51 mmol) at rt for 18 h. Water was then added, and the resulting aqueous mixture was extracted twice with ethyl acetate. The combined organic extracts were washed with aqueous lithium chloride (10%) followed by water, dried over magnesium sulfate, filtered, and concentrated in vacuo to provide N-(5- bromo-2-chloropyridin-3-yl)-N-methylmethanesulfonamide (91.2 mg, 0.304 mmol, 90% yield) as a light yellow solid. ¹H NMR (400 MHz, CDCI₃) δ 8.46 (d, 1H), 8.00 (d, 1H), 3.32(s, 3H), 3.07 (s, 3H): MS (EI) for C₇H₈BrClN₂O₂S: 299, 301, 303 (Br+CI isotopes, MH⁺).

### Reagent Preparation 29: 5-bromo-2-chloro-3-(difluoromethoxy)pyridine

To a solution of 5-bromo-2-chloropyridin-3-ol (150 mg, 0.72 mmol) in DMF (5mL) was added potassium carbonate (298 mg, 2.2 mmol). The mixture was heated to 70° C. and bromodifluoromethane was bubbled through for 3 min. After cooling to rt. water was added, and the resulting aqueous mixture was extracted twice with ethyl acetate. The organic extracts were washed with aqueous lithium chloride (1.0%): followed by water, dried over magnesium sulfate, filtered, and concentrated in vacuo to provide 5-bromo-2-chloro-3- (difluoromethoxy)pyridine (159 mg, 0.61 mmolm 85% yield) as a brown oil. ¹H NMR (400 MHz, CDC₃) δ 8.36 (d, 1H), 7.76 (d, 1H), 6.61 (t, 1H); MS (EI) for C₆H₃BRclF₂NO: 258(M⁺).

### Reagent Preparation 30: N-(5-bromo-2-ethoxypyridin-3-Yl)methanesulfonamide

STEP 1: A solution of 5-bromo-2-chloro-3-nitropyridine (100 mg, 0.42 mmol) and 1.8-diazabicylo[5,4,0]undee-7-ene (315 uL, 2.11 mmol) in ethanol (1 mL) was heated to 50° C. for 50 min and then cooled to rt. Water was added and the resulting aqueous mixture was extracted twice with ethyl acetate. The combined organic extracts were washed with 1 N HC1, dried over magnesium sulfate, filtered, and concentrated in vacuo. The residue was purified by flash chromatography (gradient, 100% hexanes to 90% hexanes : 10% ethyl acetate) to provide 5-bromo-2-ethoxy-3-nitropyridine (52.2 mg, 0.211 mmol, 50% yield) as a yellow oil. ¹H NMR (400 MHz, CDCl₃) δ 8.42 (d, 1H), 8.36 (d, 1H), 4.55 (q, 2H), 1.45 (t, 3H); MS (EI) for C₇H₇BrN₂O₃: 246, 248 (M).

STEP 2: To a solution of 5-bromo-2-ethoxy-3-nitropyridine (75.2 mg, 0.304 mmol) in ethyl acetate (3 mL) was added tin(II) chloride (289 mg, 1.52 mmol), and the mixture was heated to reflux for 2 h. After cooling to rt, 50% aqueous sodium hydroxide was added dropwise until a sticky brown solid completely formed. Sodium sulfate was then added, and the mixture was stirred for several minutes. The solids were then removed by filtration. The filtrate was dried over sodium sulfate, filtered, and concentrated in vacuo to provide 5-bromo-2-ethoxypyridin-3-amine (53 mg, 0.25 mmol, 80% yield) as a dark blue film, ¹H NMR (400 MHz, CDCI₃) δ 7.56 (d, 1H), 6.97 (d, 1H), 4.37 (q, 2H), 3.85 (br s, 2H), 1.40 (dd, 3H); MS (EI) for C₇H₉BrN₂O: 217, 219 (Br isotopes, MH⁺).

STEP 3: A solution of 5-bromo-2-ethoxypyridin-3-amine (53 mg, 0.25 mmol) and diisopropylethylamine (96 uL, 0.55 mmol) in dichloromethane (1 mL) was cooled to 0° C. and methanesulfonyl cchloride (39 uL, 0.5 mmol) was added. The mixture was allowed to warm to rt over 15 h, and then water was added. The resulting mixture was extracted with dichloromethane. The organic extract was dried over magnesium sulfate, filtered, and concentrated in vacuo. The residue was dissolved in methanol (500 uL) and dioxane (500 uL), and then sodium hydroxide (2 M, 190 uL, 0.38 mmol) was added. The mixture was heated to 60° C. and 3 drops of aqueous sodium hydroxide (50%) were added. After stirring a further 30 min, the mixture was cooled to rt. -Dilution with water was followed by acidification wilh aqueous citric acid (10%) and then two extractions with ethyl acetate. The combined organic extracts were washed with water, dried over magnesium sulfate, filtered, and concentreated vacuo. The residue was purified by flash chromatography (gradient 100% hexanes to 70% hexanes:30% ethyl acetate) to provide N-(5-bromo-2-ethoxpyridin-3- yl)methanesulfonamide (32.1 mg, 0.11 mmol, 43% yield) as a colorless film. ¹H NMR (400 MHz, CDCI3) δ 7.95 (d, 1H), 7.89 (d, 1H), 6.75 (br s, 1H), 4.42 (q, 2H), 3.05 (s, 3H), 1.41 (t, 3H); MS (EI)for C⁸H₁₁BrN₂O₃S: 295, 297 (Br isotopes, MH⁺).

Using analogous synthetic techniques and substituting with alternative starting reagents in step 1 the following reagent was prepared. Alternative starting materials were obtained commercially unless otherwise indicated.

N-(2-(Benzyloxy)-5bromopyridin-3-yl)methanesulfonamide. Prepared according to the methods described in reagent preparation 30 using benzyl alcohol in step 1. ¹H NMR (400 MHz, CDCl₃) δ 8.00 (d, 1H), 7.91 (d, 1H), 7.44-7.34 (m, 5H), 6.71 (br s, 1H), 5.40 (s, 2H), 2.99 (s, 3H): MS (EI) for C₁₃H₁₃BrN₂O₃S: 357, 359 (Br isotopes, MH⁺).

### Reagent Preparation 31: N-(2-amino-5-brmopyridin-3-yl)methanesulfonamide

STEP 1: To a solution of 5-bromo-3-nitropyridin-2-amine (218 mg, 1 mmol) in THF (5 mL) was added DMAP (183 mg, 1.5 mmol) and di-tert-butyl dicarbonate (655 mg, 3 mmol). After stirring 40 min at rt. the volatile materials were removed in vacuo, and the resulting residue was purified by flash chromatography (gradient, 100% hexanes to 70% hexanes: 30% ethyl acetate). The isolated material indicated the addition of two Boc groups by ¹H NMR. This material was dissolved in ethyl acetate (8 mL) and was treated with excess N,N-dimethylethylenediamine. After stirring for 17 h at rt. The reaction mixture was diluted wiih ethyl acetate. The resulting solution was washed with aqueous citric acid (10%) followed by water, dried over magnesium sulfate, filtered, and concentrated in vacuo to provide tert-butyl 5-bromo-3nitropyridine-2-ylcarbamate (27.0 mg, 0.85 mmol, 85%. yield) as an orange solid. ¹H NMR (400 MHz, CDCl3) δ 9.48 (br s, 1H), 8.74 (d, 1H), 8.63 (d, 1H), 1.56 (s, 9H); MS (EI) for C₁₀H₁₂BrN₃O₄: 316, 318 (Br isotopes, MH).

STEP 2: Iron powder (293 mg, 5.2 mmol) was added to a solution of tert-butyl 5- bromo-3-nitropyridin-2-ylcarbamate (167 mg, 0.52 mmol) in acetic acid (2.5 mL). The mixture was stirred at 60° C. for 1 h 20 in before cooling to rt. The mixture was then diluted with ethyl acetate, and solids were removed by filtration through celite. The filtrate was washed with water followed by saturated aqueous sodium bicarbonate. The organic phase was dried -oyer-magnesium sulfate, filtered, and concentrated in vacuo to provide tert-butyl 3- amino-5-bromopyridin-2-ylcarbamate (96.3 mg, 0.33 mmol 64% yield) as a gray solid. ¹H NMR (400 MHz, CDCI₃) δ 7.83 (d, 1H), 7.20 (d, 1H), 6.95 (br s, 1H), 4.42(br s, 2H), 1.51 (s, 9H): MS (EI) for C₁₀H₁₄BrN₃O₂:2.32, 234 (Br isotopes, MH⁺-t-butyl).

STEP 3: A solution of tert-butyl 3-amino-5-bromopyridin-2-ylcarbamate (96.3 mg, 0.33 mmol) and diisopropylethylamine (128 11L. 074 mmol) iii dichloromethane (2 mL) was cooled to 0° C., and to it was added methanesulfonyl chloride (52 uL, 0.67 mmol). The mixture was allowed to warm 10 rt over 2 h. The mixture was then diluted with dichloromethane and was then washed with aqueous citric acid (10%) followed by water. The organic phase was then dried over magnesium sulfate, filtered, and concentrated in vacuo. The residue, was purified by flash chroniatography (gradient, 100% hexanes to 70% liexancs : 30% ethyl acetate) to provide tert-butyl 5-bromo-3-(N- -(methylsulfonyl)methylsulfonamido)pyridin-2-ylcarbamate (77 mg, 0.17 mmol, 52% yield) as a colorless film. ¹H NMR (400 MHz, CDCl₃) δ 8.64 (d, 1H), 7.79 (d, 1H), 7.10 (s, 1H), 3.44 (s, 6H), 1.52 (s, 9H); MS (EI) for C₁₂H₁₈BrN₃O₆S₂: 388, 390 (Br isotopes, MH⁺-t-butyl).

STEP 4: A solution of tert-butyl 5-bromo-3-(N- (methylsulfonyl)methylsulfonamido)pyrindin-2-ylcarbamate (68 mg, 0.5 mmol) and N,N-dimethylethylenediame (169uL, 1.5 mmol) in dioxane (1 mL) was stirred at rt for 70 min. After diluting with ethyl acetate, the mixture was washed with queous acid (10%) followed by water. The organic phase was then dried over magnesium sulfate, filtered, and concentrated in vacuo. The residue was then diluted with dichloromethane which was then washed with 1 N HCI. After partitioning, the organic phase,was dried over magnesium sulfate, filtered, and concentrated in vacuo lo provide tert-butyl 5-bromo-3- (methylsulfonamido)pyridin-2-ylcarbainate (57 mg, 0.15 mmol, quantitative yield) as a white solid. ¹H NMR (400 MHz, CDCl₃) δ 8.24 (d, 1H). 8.07 (d, 1H), 2.98 (s, 3H), 1.54 (s, 9H): MS (EI) for C₁₀H₁₆BrN₃O₄S: 310, 312 (Br isotopes, MH⁺-t-butyl).

STEP 5: A solution of tert-butyl 5-bromo-3-(methylsulfonamido)pyridin-2- ylcarbamate (57 mg, 0.15 mmol) in methanol (1 mL) and HCI (4 M in dioxane, 375 uL, 1.5 mmol) was heated to 60° C. for 90 min. The volatile materials were then removed in vacuo to provide N-(2-amino-5-bromopyridin-3-yl)methanesulfonamide as its hydrochloride salt in quantitative yield. ¹H NMR (400 MHa, DMSO-d6) δ 9.10 (brs, 1H), 7.95 (d, 1H), 7.54 (d, 1H), 6.42 (br s, 1H), 3.02 (s, 3H): MS (EI) for C₆H₈BrN₃O₂S: 266, 268 (Br isotopes, MH⁺).

### Reagent Preparation 32: 5-bromo-1-(tetrahydro-2H-pyran-2-yl)-1H-pyrazolo[3,4-b]pyridine

To a solution of 5-bromo-1H-pyrazolo[3,4-b]pyridine (1.4 g, 7.2 mmol) and dihydropyran (3.3 mL, 36.0 mmol) in tetrahydrofuran (20 mL) was added (±) camphorsulfonic acid (250 mg) and the reaction nrixture was stirred at 65° C. for 16 hours. After cooling lb robin temperature it was diluted with ethyl acetate (25.0 mL), washed with saturated aqueous, sodium bicarbonate (2 x 100 mL) and brine (100 mL), dried over sodium sulfate, filtered and concentrated. Gradient cbluinri chromatography (10% to 30% ethyl acetate in hexane) provided 5-bromo-1-(tetrahydro-2H-pyran-2-yl)-1H-pyrazolo[3,4- b]pyridine (1.8 g, 90%), MS (EI) for C₁₁H₁₂BrN₃O: 283 (MH⁺).

### Reagent Preparation 33: 2-Amino-5-bromo-N,N-dimethylnicotinamide

To a suspension of 2-amino-5-bromonicotinic acid (0.35 g. 1-.61 mmol) in tetrahydrofuran (5 mL) was added dimethylamine (0.8 mL of a 2M solution in tetrahydrofuran, 1.60 mmol), diethylphosphoryl cyanide (0.29 g, 1.77 mmol), and triethylamine (0.34 g, 3.38 mmol) at 0° C. The mixture was stirred at 0° C. for 30 min and then at room temperature for 4 h. Concentration and purification by column chromatography on silica (5-10% methanol in dichloromethane) gave the title Compound as a white solid. MS (EI) for C₈H₁₀BrN₃O: 244 (MH⁺).

Reagent Preparation 34: 5-Bromo-3-(etthylsulfonyl)pyridin-2-amine

STEP 1: 2-Amino-5-bromopyridine-3-sulfonyl chloride (94 mg, 0.35 mmol) was taken into THF (2 mL) followed by addition of anhydrous hydrazine (40 uL, 1.4 mmol) and the mixture was stirred for 10 minutes at room temperature. The mixture was concentrated and dried to give 2-amino-5-bromopyridine-3-sulfonohydrazide as a white solid, which was then taken into ethanol (2 mL) followed by addition of spdium acetate (320 mg, 3.9 mmol) and ethyl iodide (140 uL. 1,75 mmol). The mixture was refluxed for 12h then cooled to room temperature and concentrated. The residue was partitioned with ethyl acetate and water and the organic phase washed with brine then dried over sodium sulfate, filtered and concentrated to give 5-bromo-3-(ethylsulfonyl)pyridin-2-amine (67 mg, 72%) as a yellow oil. MS (EI) for C₇H₉N₂SO₂Br: 265, 267 (MH⁺).

Using analogous synthetic techniques and substituting with alternative starting reagents in step 1 the following reagents were prepared.

5-Brmoo-3-(methylsulfonyl)pyridin-2-amine. Synthesized according to the method of reagent preparation 34 using iodomcthane. GCMS (EI) for C₆H₇N₂SO₂Br: 250, 252 (M⁺).

3-(2-amino-5-bromopyridin-3-ylsulfonyl)propane-1,2-diol. Synthesized according to the method of reagent preparation 34 using 3-bromopropane-1,2-diol followed by silica gel chromatography using ethyl ether then ethyl acetate as eluent. MS (EI) for C₇H₉N₂SO₂Br: 311, 313 (MH⁺).

3-(2-amino-5-bromopyridine-3-ylsulfonyl)propan-1-ol. Synthesized according to the method of reagent preparation 34 using 3-bromopropan-1-ol followed by sillca gel chromatography using ethyl ether as elueiit. MS (EI) for C₇H₉N₂SO₂Br: 295, 297 (MH⁺).

(S)-3-(2-amino-5-bromopyridin-3-ylsulfonyl)-2-methylpropan-1-ol. Synthesized according to the method of reagent preparation 34 using (S)-3-bromo-2-methylpropan-1-ol followed by silica gel chromatography using 4:1 ethyl ether:hexanes as eluent. MS (EI) for C₇H₉N₂SO₂Br: 309, 311 (MH⁺).

(R)-3-(2-amino-5-bromopyridin-3-ylsulfonyl)-2-methylpropan. Synthesized according ip the methed of reagent preparation 34 using (R)-3-brbriiP-2-mcthyIpropan-1 -ol followed by silica gel chromatography using 4:1 ethyl ether:hexanes as eluent. MS (EI) for C₇H₉N₂SO₂Br: 309, 311 (MH⁺).

### Reagent Preparation 35: 6-bromo-2-methyl-1-({[2-(trimethylsilyl)ethyl[oxy}methyl)-1H-imidazo[4,5-b]pyridine

To a solution of 6-bromo-2-methyl-1 H-imidazo[4,5-b]pyridine (3.0 g, 14.1 mmol) in a mixture of N,N-dimethylformamide and tetrahydrofuran (30 mL, 2:1) at 0° C. was added 60% sodium hydride in mineral oil (0.68 g,17.0 mmol) and the reaction mixture was stirred for 30 minutes, followed by the addition of 2-(trimethylsilyl)ethoxymethyl chloride (2.7 mL, 14.9 mmol). The reaction mixture was stirred for 16 hours at room temperature then it was quenchcd by the careful additipn of water and diluted with ethyl acetate (250 mL), washed with brine (3x 150 mL), dried over sodium sulfate, filtered and concentrated. Gradient column chromatography (10% to 30% ethyl acetate in hexane) provided 6-bromo-2-methyl- 1-({[2-(trimeihylsilyl)ethyl]oxy)methyl)-1H-imidazo[4,5-b]pyridine (4.4 g, 92%). ¹H NMR (400 MHz, CDCl₃): 8.41 (s, 1H), 8.12 (s, 1H), 5.67 (s, 2H), 3.62 (m, 2H), 2.76(s, 3H), 0.96 (m, 2H), 0.00 (s, 9H), MS (EI) for C₁₃H₂₀BrN₃OSi: 342, 344 (MH⁺, Br iotope pattern).

### Reagent Preparation 36: 6-bromo-N-ethyl-3-(methoxymethyl)-3H-imidazo[4,5- b]pyridin-2-amine and 6-bromo-N-ethyl-N,3-bis(methoxymetlhyl)-3H-imidazo[4,5- b]pyridin-2-amine

Step 1: To a cooled (0° C.) solution of 5-bromopyridine-2,3-diamine (5.0 g, 27 mmol) in NMP (20 mL) was added isothiocyanatoethane (2.3 mL, 26 mmol). The resulting solution was heated (65° C.) for four hours and then cooled to ambient temperature before 1,3-diisopropylcarbodiimide (4.2 mL, 27 mmol) - was added. The reaction mixture was stirred for 18 hours, diluted with water and the resulting suspension was collected by filtration. Trituration with ethyl acetate provided 6-bromo-N-ethyl-3H-imidazo[4,5-b]pyridin-2-amine (4.8 g, 75% yield) as a brown solid. ¹H NMR (400 MHz, d₆-DMSO) δ 11.41 (bs, 1H), 7.91 (s, 1H), 7.53 (s, 1H), 7.17 (s, 1H), 3.33 (q, 2H), 1.17 ([, 3M): MS (ES) for C₈H₉BrN₄: 241 (MH⁺).

Step 2: To a cooled (0° C.) solution of 6-bromo-N-ethyl-3H-imidazo[4,5- b]pyridin-2-amine (0.36 g, 1.5 mmol) in DMF was added NaH (60% dispersion in mineral oil, 0.060 g, 1.5 mmol) portion wise over 15 minutes. The reaction --mixture was stirred for 15 minutes and then chloro(methoxy)methane (0.12 mL, 1.5 mnipl) was added dropwise over 15 minutes. The resulting slurry svas allowed to warm.io ambient temperature and was stirred for two hours and was partitioned between ethyl acetate and saturated aqueous sodium bicarbonate. The organic layer was washed with brine, dried over magnesium sulfate, filtered and concentrated in vacuo. Purification by silica gel chromatography provided both 6-bromo- N-ethyl-N,3-bis(methoxymethyl)-3H-imidazo[4,5-b]pyridin-2-aniine (0.091 g, 18%) and 6- bromo-N-ethyl-3-(methoxymetthyl)-3H-imidazo[4.5-b]pyridin-2-amine (0.15 g,. 35% yield). Bisprotected product: MS (ES) for C₁₂H₁₇BrN₄O₂: 329 (MH⁺). Monoprotected product: ¹H NMR (400 MHz, CDCI3) δ 8.03 (d, 1H), 7.73 (d, 1H), 5.42 (s, 2H), 4.98 (s, 1H), 3.59 (q, 211), 3.36 (s, 3H), 1.34 (t, 3H); MS (ES) for C₁₀H₁₃BrN₄O: 285 (MH⁺).

### Reagent Preparation 37: 7-Bromo-2H-pyrido[2,3-e][1,2,4]thiadiazin-3(4H)-one 1,1-dioxide

STEP 1: 2-Amino-5-bromopyridine-3-sulfonyl chloride (reagent preparation 25) (95.5 mg, 0.35 mmol) was treated with 0.5M ammonia in dioxane solution (7 mL) and the mixture was stirred for 1 h at room temperature. Concentrated aqueous ammonia (2 mL) was then added to the mixture then stirred aii additional 12h. The mixture was then concentrated and the residue suspended in water (5 mL). The solid was collected by filtration and dried to give 2-amino-5-bromopyridine-3-sulfonamide (55.7 mg, 89%).

STEP 2: 2-Amino-5-bromopyridine-3-sulfonamide as obtained above (0.22 mmol) was taken/into THF (2 mL) followed by addition of diisopropylethylamine (115uL. 0.66 mmol). Phosgene (20W % in toluene, 120uL, 0.22 mmol) was added carefully and the mixture was allowed to stir for 1 h at room temperature. The mixture was partitioned with ethyl acetate and 0.5 M aqueous hydrochloric acid. The organic phase was then extracted once with saturated aqueous sodium bicarbonate. The organic layer was discarded and the aqueous phase carefully acidified to pH 1-2 with concentrated aqueous hydrochloric acid. The aqueous mixture was then extracted once with ethyl acetate, dried over sodium sulfate, filtered and concentrated to give 7-bromo-2H-pyrido[2,3-e][1,2,4]thiadiazin-3(4H)-one 1,1-dioxide (17.3 mg, 28%) as a solid. MS (EI) for C₆H₄N₃O₃SBr: 277, 279 (M).

### Reagent Preparation 38: 2-amino-5-bromopyridine-3.sulfonic acid

STEP 1: 2-Amino-5-bromopyridine-3-sulfonyl chloride (100 mg, 0.37 mmol) was taken into 1:1 aqueous dioxanc (3 mL) and the mixture was basified lo pH 14 by drop wise addition of 50% aqueous sodium hydroxide solution. The mixture was warmed to 75° C. for 0.5h then cooled to room temperature and concentrated. The residue was taken into water (2 mL) and carefully acidified to pH 1-2 by concentrated aqueous hydrochloric acid addition and cooled to 0° C. After 1 h at 0° C. the crystalline solid obtained was collected by filtration and dried to give 2-amino-5-bromopyridine-3-sulonic acid as a solid. ¹H NMR (DMSO-d₆): 8.24 (d, 1H), 8.06 (d, 1H), MS (EI) for C₅H₅N₂SO₃Br: 253, 255 (MH⁺, Br pattern).

### Reagent Preparation 39: N-(5-bromo-2-(dimethlamino)pyridin-3-yl)methanesulfonamide

STEP 1: 5-Bromo-2-chloro-3-nitropyridine (J. Heterocyclic Chem. 2003, 40, 261) (128 mg, 0.54 mmol) was taken into THF (0.25 mL) followed by addition of 4 W % aqueous dimethylamine (0.25 mL) and the resulting solution was stirred for 1 h at room temperature. The mixture was then partitioned with ethyl ether and 1 M aqueous hydrochloric acid. The organic solution was then washed with additional 1 M aqueous hydrochloric acid (3x) then dried over magnesium sulfate, filtered and concentrated to give 5-bromo-N,N-dimethyl-3- nitropyridirn-2-amine. MS (EI) for C₇H₈N₃O₂Br: 246, 248 (MH⁺, Br pattern).

STEP 2:5-Bromo-N,N-dimethyl-3-nitropyiidin-2-amine as obtained in step 1 (0.54 mmol) was taken into ethyl acetate (10 mL) followed by addition of tin (II) chloride (522 mg, 2.8 mmol) and the mixture was heated to reflux for 15 minutes then cooled to room temperature. 50W % aqueous sodium hydroxide was added drop wise to the mixture until a precipitate formed then solid sodium sulfate was added. The mixture was filtered and the filter cake washed with ethyl acetate. The organic filtrate was concentrated to give 5-bromo- N2,N2-dimethylpyridince-2,3-diamine (53 mg, 45%) was an amorphous residue. MS (EI) for C₇H₁₀N₃Br: 216. 218 (MH⁺, Br pattern).

STEP 3:5-Bromo-N2,N2-dimethyipyridine-2,3-diamine (53 mg, 0.25 mmol) was taken into THF (2 mL) followed by addition of diisopropylethylamine (213 uL, 1.25 mmol) and methanesulfonyl chloride (95 ul, 1.25 mmol). The mixture was allowed to stir, for 48 h at room temperature then partitioned with ethyl acetate and water. The organic phase was washed with brine then dried over sodium sulfate, filtered and concentrated. The residue was taken into methanol (3 mL) followed by addition of potassium hydroxide (108 mg, 10 eq) in a minimum of water. The mixture was stirred for 15 minutes at room temperature then partitioned with ethyl acetate and 10% aqueous citric acid. The organic solution was dried over magnesium sulfate, filtered and concentrated. The residue was purified by silica gel chromatography to give N-(5-bromo-2-(dimethylamino)pyridine-3-yl)methanesulfonamide (27.9 mg, 39%): MS (EI) for C₈H₁₂N₃SO₂Br: 294, 296 (MH⁺, Br pattern).

Using analogous synthetic techniques and substitutihg with alternative starting. reagents, in step 1 the following reagents were prepared.

N-(,2-Benzylamino)-5bromopyridin-3-yl)methanesulfonamide. Synthesized according to the method of reagent preparation 39 using benzylamine in step 1, MS (EI) for C₁₃H₁₄N₃SO₂Br: 356, 358 (MH⁺, Br pattern).

N-(5-Bromo-2-(phenylamino)pyridin-3-yl)methanesulfonamide. Synthesized according to the method of reagent preparation 39 using heat aniline at 75° C. in step 1. MS (EI) for C₁₂H₁₂N₃SO₂Br: 342, 344 (MH⁺, Br pattern).

N-(5-Bromo-2-(methylamino)pyridin-3-yl)methanesulfonamide. Synthesized according to the method of f reagent preparation 39 using methyamine in step 1. MS (EI) for C₇H₁₀N₃SO₂Br: 280, 282 (MH⁺, Br pattern).

### Reagent Preparation 40: 1,1-dimethylethyl {(3S)-1-[(5-bromo-2-hydroxypyridin-3- yl)sulfonyl]pyrrolidin-3-yl}carbamate and 1,1-dimethylethyl](3S)-1-({5-bromo-2-[3S)- 3-({[[1,1-dimethylethyl)oxy]carbonyl{amino)pyrrolidin-1-yl]pyridin-3- yl }sulfonyl)pyrrolidin-3-yl ]carbamate.

STEP 1: To a solution of 3-amino-5-bromo-2-chloropyridine (0.23 g, 1.1 mmol) in acetoniirile (3.0 mL) at −15° C. was added a solution of sodium ntrite (0.091 g, 1.3 mmol) in water (1.20 mL), followed bytiie addition of concentrate hydrochloric acid (.1.8 mL, 21.3mmol) and the reaction mixture was stirred for 5 minutes. A 30 wt % solution of sulfur dioxide in acetic acid 3.0 mL, 1.3 mmol) was prepared and introduced into the reaction mixture, followed by the addition of a solution of copper(II) chloride 0.09 i g, 0.68 mmol) in water (1.2 mL). The stirring was continued for an additional 3 hours at −5° C. The pH of the mixture was adjusted to 8 by the addition of a solution of potassium hydrogenphosphate and 2M aqueous sodium hydroxide and partitioned with ethyl acetate (50 mL). The organic layer was separated and washed with water (10 mL) and brine (10 mL), dried over sodium sulfate, filtered and concentrated to give 5-bromo-2-chloropyridine-3-sulfonyl chloride (0.20 g, 63%).

STEP.2: A mixture of 5-bromo-2-chloropyridine-3-sulfonyl chloride (0.19 g, 0.65 mmol), (3S)-(-)-3-(tert-butoxycarbonylamino)pyrrolidine (0.18 g, 0.98 mmol) and N,N-diisopropylethylamine (0.34 mL, 1.95 mmol) in dichloromethane (1:5 mL) was stirred for 16 hours at room temperature. The reaction mixture was partitioned between dichloromethane (50 mL) and brine (10 mL). The organic layer was separated, dried over sodium sulfate, filtered and concentrated. The resulting crude product was-dissolved in a mixture of 1,4- dioxaiie (1.5 mL) and 2M aqueous sodium hydroxide (1.5 mL) and stirred al 100° C. for 2 hours. After cooling to room temperature the reaction mixture was concentrated and the residue was partitioned between brine (20 mL) and ethyl acetate (50 mL). The organic layer was separated and washed with brine (20 mL), dried over sodium sulfate, filtered and concentrated. Gradient flash chrmatography (25% to 50% ethyl acetate in hexane) followed by 10% methanol in dichloromethanc provided 1,1-dimethylethyl [(3S)-1-({5-bromo-2-[(3S)- 3-({[(1,1-dimethylethyl)oxy]carbonyl}amino)pyrrolidin-1-yl]pyridin-3- yl}sulfonyl)pyrrolidin-3-yl]carbamate (80 mg, 21%), MS (EI) for C₂₃H₃₆BrN₅O₆S: 591 (MH⁺); and 1,1 -dimethylethyl {(3S)-1-[(5-bromo-2-hydroxypyridin-3-yl)sulfonyl]pyrrolidin- 3-yl carbamate (35 mg, 13%); MS (EI) for C₁₄H₂₀BrN₃O₅S: 423 (MH⁺).

### Reagent Preparation 41: 4-[(2-amino-5-bromopyridin-3-yl)sulfonyl]-2-methylbutan-2-ol and 4-[(2-amino-5-bromopyridin-3-yl)sulfinyl]-2-methylbutan-2-ol

STEP 1: To a solution of 2-amino-5-bromopyridine-3-sulfonyl chloride (reagent preparation 25, step 1) (0.40 g, (1.47 mmol) in a mixture of 1.4-dioxane (8.0 mL) and water (1.0 mL) was added triphenylphosphine (1.64 g, 6.25 mmol) and the reaction mixture was stirred for 50 minutes at room temperature. Potassium carbonate (0.35 g, 2.50 mmol) was introduced, followed by 4-bromo-2-methyl-2-butanol (Tetrahedron Letters 2000, 41(38), 7337-7340) (0.31 g, 1.86 mmol) and the reaction mixture was stirred at 80° C. for 16 hours. After cooling lo room temperature the reaction mixture was concentrated and the residue was partitioned between brine (50 mL) and ethyl acetate (100 mL). The organic layer vyas separated and washed with brine (50 mL), dried over sodium sulfate, filtered and concentrated. Gradient flash chromatography (25% to 50% ethyl acetate in hexane) provided 4-[(2-amino-5-bromopyridin-3-yl)thio]-2-methylbulan-2-ol (0.18 g, 42%); MS (EI) for C₁₀H₁₅BrN₂OS: 292 (MH⁺).

STEP 2A: To a solution of 4-[(2-amino-5-bromopyridin-3-yl)thio]-2-methylbutan- 2-ol (90 mg, 0.31 mmol) in a mixture of methanoL(750 μL), acetone (750 μL) and water (450 μL) was added potassium peroxymonosulfate (285 mg, 0.46 mmol) and the reaction mixture was stirred for 15 minutes at room temperature. The reaction mixture was partitioned between water (20 mL) and ethyl acetate (50 mL). The organic layer was separated and washed with water (20 mL) and brine (20 mL), dried over sodiuni sulfate, filtered and concentrated. Purification by flash chromatography (35% to 80% ethyl acetate in hexane) gave 4-[(2-amino-5-bromdpyridin-3-yl)sulfonyl]-2-methylbutan-2-ol (48%); MS (EI) for C₁₀H₁₅BrN₂O₃S: 323 (MH⁺).

Step 2B: To a solution, of 4-[(2-amino5-bromopyridin-3-yl)thio]-2-methylbutan- 2-ol (83 mg. 0.28 mmol) in a mixture of methanol (750 μL), acetone (750 μL) and water (450 μL) was added potassium peroxymonosulfate (131 mg, 0.21 mmol) and the reaction mixture was stirred for 90 minutes at 0° C. The reaction mixture was partitioned between water (20 mL) and ethyl acetate (50 mL). The organic layer was separated and washed with water (20 mL) and brine (20 mL), dried over sodium sulfate, filtered and concentrated. Purification by flash chromatography (35% to 80% ethyl acetate in hexane) gave 4-[(2-amino-5- bromopyridin-3-yl)sulfinyl]-2-methylbutan-2ol (52 mg, 60%); MS (EI) for C₁₀H₁₅BrN₂O₂S: 308 (MH⁺).

Using analogous synthetic techniques and substituting with alternative starting materials in step 1 the following reagents of the invention were prepared. Alternative starting materials were obtained commercially unless otherwise indicated.

(2S)-3-[(2-amino-5-bromopyridin-3-yl)sulfonyl]-2-methylpropan-1-ol. Prepared according to the method of reagent preparation 41 by using (S)-(+)-3-bromo-2-methyl-1- propanol in step 1. MS (EI) for C₉H₁₃BrN₂O₃S: 310 (MH⁺).

(2S)-3-[(2-amino-5-bromoyridin-3-yl)sulfinyl]-2-methylpropan-1-ol. Prepared according to the method of reagent preparation 41 by using (S,)-(+)-3-bromo-2-methyl-1- propanol in step 1. MS (EI) for C₉H₁₃BrN₂O₃S: 294 (MH⁺).

### Reagent Preparation 42: (4-chloro-5,6,7,8-tetrahydroquinazolin-7-yl)methanol.

Ozone was bubbled through a cooled (−78° C.) solution of 4-chloro-7-vinyl- 5,6,7,8-tetrahydroquinazoline(reagent preparation 3, 0.35 g, 1.8 mmol) in methanol (5 mL) and dichloromcthane (30 mL) until a blue color persisted. The solution was then sparged with N₂for 10 minutes and sodium borohydride (14 g, 3.6 mmol) was added portionwise. After 30 minutes the reaction mixture was partitioned between dichloromcthane and saturated aqueous sodium bicarbonate. The organic layer was washed with brine, dried over magnesium sulfate, filtered and then concentrated in vacuo to provide (4-chloro-5,6,7,8- tetrahydroquinazolin-7-yl)methanol (0.32 g, 90% yield) as a waxy solid. MS (ES) for C₉H₁₁ClN₂O: 1.99 (MH⁺).

### Reagent example 43: 1-(4-chloro-5,6,7,8-tetrahydroqiiinazlin-7-yl)ethanol

Step 1: Ozone was bubbled through a cooled (−78° C.), solution of 4-chloro-7- vinyl-5,6,7,8-tetrahydroquinazoline (reagent preparation 3, 0.38 g, 2.0 mmol) in dichloromethane (45 mL) until a blue color persisted. The sdlution was then sparged with N₂ for 10 minutes and triphenylphosphine (0.52 g. 2.0 mmol) was added portionwise. After one hour, the reaction mixture was partitioned between dichloromethane and saturated, aqueous sodium bicarbonate. The organic layer was washed with brine, dried oyer magnesium sulfate, filtered and then concentrated in vacuo. Purification by silica gel chromatography provided 4- chloro-5,6,7,8-tetrahydroquinazoline-7-carbaldehyde (0.33 g, 85% yield) as a viscous oil. MS (ES) for C₉H₉ClN₂O: 197 (MH⁺).

Step 2: To a cooled (0° C.) solution of 4-chloro-5,6,7,8-tetrahydroquinazoline-7- carbaldehyde (0.10 g, 0.51 mmol) in THF (5 mL) was added a solution of MeMgBr (3.0 M in ethyl ether, 0.40 mL, 1.2 mmol). The resulting mixture was stirred at ambient temperature for 30 minutes and then partitioned between ethyl acetate and saturated sodium bicarbonate. The organic layer was washed with brine, dried over magnesium sulfate, filtered and concentrated in vacuo. Purification by silica gel chromatography provided 1-(4-chloro-5,6,7,8- tetrahydroquinazolin-7-yl)ethanol (0.09 g, 83% yield) as a waxy solid. MS (ES) for C₁₀H₁₃ClN₂O: 213(MH⁺).

### Reagent example 44: 4-chloro-7-(methoxymethyl)-5,6,7,8-tetrahydroquiniizoline

To a slurry of (4-chloro-5,6,7,8-tetrahydroquinazolin-7-yl)methanol (reagent preparation 42, 0.80 g, 0.40 mmol), potassium carbonate (0.11 g, 0,81 mmol) and THF (15 mL) was added iodomcthane (0.09 mL, 0.60 mmol). The reaction mixture was stirred for 18 hours and then partitioned between ethyl acetate and water. The organic layer was washed with brine, dried over magnesium sulfate, filtered and concentrated in vacuo. Purification by silica gel .chromatography provided 4-chloro-7-(methoxymethyl)-5,6,7,8- tetrahydroquinazoline (0.03 g, 35% yield) as a waxy solid. MS (ES) for C₁₀H₁₃ClN₂O: 213 (MH⁺).

### Reagent Preparation 45: 2-(azidomethyl)-4-chloro-6,6-dimethyl-5,6,7,8-tetrahydroquinazoline

STEP 1: To a solution of 2-(chloromethyl)-6,6-dimethyl-5,6,7,8- tetrahydioquinazolin-4(3H)-one (150 mg, 0.66 mmol, reagent preparation 17) in DMF (3 mL) was added sodium azide (215 mg, 3.3 mmol). The resulting mixture was stirred at rt for 35- min. Water was added and the resulting mixture was extracted twice with ethyl acetate. The combined organic extracts were washed with aqueous lithium chloride (10.%), dried over magnesiumsulfate, filtered, and concentrated in vacuo to provide 2-(azidomethyl)-6,6- dimethyl-5,6,7,8-tettahydroquinalin-4(3H)-one (1,51 mg, 0.65 mmol, 98% yield) as a waxy yellow solid. ¹H NMR (400 MHz, CDCl₃) δ 11.70 (br s, 1H), 4.41 (s, 2H), 2.66(t, 2H), 2.33 (s, 2H), 1,58 (t, 3H), 1.00 (s, 6H): MS (EI) for C₁₁H₁₅N₁₅N₅O: 234(MH⁺).

STEP 2: A solution of 2-(azidomethyl)-6,6-dimethyl-5,6,7,8- tetrahydroquinazolin-4(3H)-one (151 mg, 0.65 mmol) in chloroform (1.2 mL) was treated with phosphorus oxychloride (600 uL) al 60° C. for 1 H 20 min. After cooling to rt, the volatile materials were removed in vacuo, and the resulting residue was dissolved in ethyl acetate. The organic solution was washed with saturated aqueous sodium bicarbonate, and the aqueous phase was back extracted with ethyl acetate. The combined organic extracts were dried over magnesium sulfate, filtered, and concentrated in vacuo to provide 2-(azidomethyl)- 4-chloro-6,6-dimethyl-5,6,7,8-tetrahydroquinazoline (136 mg, 0.54 mmol, 83% yield) as an orange oil. ¹H NMR (400 MHz, CDCl₃) δ 4.47 (s, 211), 2.94 (t, 2H), 2.55 (s, 2H), 1.68 (t, 2H), 1.05 (s, 6H): MS (EI) for C₁₁H₁₄ClN_{b}: 252 (MH⁺).

### Reagent Preparation 46: 1-(4-chloro-6,6-dimethyl-5,6,7,8-tetrahydroquazolin-2-yl)-N,N-dimethylethanamine

STEP 1: To a solution of dimcthylamine (2M solution in tetrahydrofurnn, 4.0 mL, 8.0 mmol) was added 2-(1-chloroethyl)-6,6-dimethyl-5,6,7,8-tetrahydroquinazolin-4-ol (synthesized according to the method of reagent preparation 18 using 2-chloropropionitrile in step 1) (50 mg, 0.21 mmol) and the reaction mixture was stirred in a sealed tube for 16 hours at 80° C. After cooling to room temperature the reaction mixture was concentrated and the residue was partitioned between brine (50 mL) and ethyl acetate (50 mL). The organic layer was separated and washed with brine (20 mL), dried over sodium sulfate, filtered and concentrated to give 2-[1-(dimethylamino)eihyl]-6,6-dimethyl-5.6,7,8;S-tetrahydroquinazolin- 4-ol (50 mg, 96%), MS (EI) for C₁₄H₂₃N₃O: 250 (MH⁺).

STEP 2: A solution Of 2-[1-(dimethylainino)ethyl]-6,6-dimethyl-5,6,7,8- tetrahydroquinazolin-4-ol (50 mg, 0.20 mmol) in a mixture of chloroform (1.5 mL) and phosphorous oxyehloridc (0.5 mL) was healed to reflux for 9.0 minutes. After cooling to room temperature the reaction mixture was concentrated and the residue was partitioned between saturated aqueous sodium bicarbonate (20 mL) and ethyl acetate (20 mL). The mixture was stirred for 15 minutes and pH was maintained above 7 by the addition of solid sodium bicarbonate. The organic layer was separated and washed with water (10 mL) and brine, dried over sodium sulfate, filtered and concentrated to give, 1-(4-chloro-6,6-dimethyl,-5,6,7,8- tetrahydroquinazolin-2-yl)-NN-dimethylethanamine (46 mg, 85%), MS (EI) for C₁₄H₂₂CIN₃: 268(MH⁺).

Using analogous synthetic techniques and substituting with alternative starting materials in step 1 the followingreagent was prepared. Alternative starting materials were obtained commercially unless otherwise indicated.

4-chloro-6,6-dimethyl-2-(1-pyrrolidin-1-ylethyl)-5,6,7,8-tetrahydroquinazoline. Prepared according to the method of reagent preparation 46 by using pyrrolidine in step 1. MS (EI) for C₁₆H₂₄ClN₃: 294 (MH⁺).

### Reagent Preparation 47: methyl 6-bromo-1H-imidazo[4,5-c]pyridin-2-ylcarbamate

A solution of 2-bromo-5-nitropyridin-4-amine (1.5 g, 6.9 mmol) in acetic acid (20 mL) was added in portions into a 75° C. suspension of iron powder (1.5 g 27 mmol) in acetic acid (20 mL). The reaction mixture was stirred at 75° C. for 2 h, cooled to room temperature, and filtered through celite. To the filtrate was added 1,3-bis(methoxycarbonyl)-2-methyl-2- thiopseudourea (1.4 g, 6.9 mmol), and the mixture was stirred at 65° C. for 60 h. The reaction mixture was cooled to room temperature and concentrated. The solid residue was triturated with dichloromethane and dried to give the title Compound (1.8 g, -quantitative yield) as an orange solid. MS (EI) for C₈H₇BrN₄O₂: 271/273 (MH⁺).

### Reagent Preparation 48: tert-butyl 3-(bis(tert-butoxycarbonyl)amino)-5-bromo-1H-indazole-1-carboxylate

To a cooled (0° C.) solution of 5-bromo-1H-indazol-3-amine (0.30 g, 1.4 mmol). DIPEA (2.5 mL, 14 mmol) and di-tert-butyl dicarbonate (1.5 g, 7.0-mmol) in THF (15 mL) was added DMAP (0.09 g, 0.70 mmol). The reaction mixture was then stirred at ambient temperature for three hours. The resulting solution was diluted with ethyl acetate (75 mL) and washed with saturated aqueous ammonium chloride (2 x 50 mL). The organic layer was washed widi brine, dried over magnesium sulfate, filtered and concentrated in vacuo. Purification by silica gel chromaltigraphy provided tert-butyl 3-(bis(tert- butoxycarbonyl)amino)-5-bromo-1H-indazole-1-carboxylate (0.44 g, 61%) as a waxy solid. ¹H NMR (400 MHz, CD.CI₃) δ 8.04 (t, 1H), 7.68 (dd, 1H), 7.66-7.58 (m, 1H), 1.53 (s, 18H), 1.43 (s, 9H); MS (EI) for C₂₂H₃₀BrN₃O₆: 512 (MH⁺).

### Reagent Preparation 49: 6-chlor-N-phenylpyrimidine-4-amine

STEP 1: 6-Chloropyrimidin-4-ol (500mg, 3.85 mmol), aniline (420 μL, 4.62 mmol), and N,N-diisopropylethylamine (1 mL) in diethylene glycol dimethyl ether (5 mL) was heated to 120° C. and stirred for 8h. The mixture was cooled to room temperature then diluted with actone:diethyl ether solution (1:1, 15 ml) to give a precipitate. The solid collected by filtration and washed with acctone then dried to afford 6- (pheylaniino)pyrimidin-4-ol (255 mg, 35.5% ), MS (EI) for C₁₀H₉N₃O: 188.2 (MH⁺).

STEP 2: 6-(Phenylamino)pyrimidin-4-ol (253 mg, 1.35 mmol) was dissolved in neat phosphorous oxychloride (5 mL) and stirred for 3h at 95° C. then cooled to room temperature and concentrated. The residue was poured into an ice water slurry and extracted with dichloromethane. The extract was washed saturated aqueous sodium bicarbonate solution, dried over sodium sulfate, filtered and the solvent evaporated to afford 6-chloro-N- phenylpyriniidine-4-amine (220 mg) which was used without further purification.

Using analogous synthetic techniques and substituting with alternative starting reagents in step 1 the following reagents were prepared.

6-Chloro-N-(4-methoxyphienyl)pyrimidin-4-amine. Synthesized according to the method of reagent preparation 49 using 4-methoxyaniline in step 1.

6-Chloro-N-(3-meythoxyphenyl)pyrimidin-4-amine. Synthesized according to the method of reagent preparation 49 using 3-methoxyaniline in step 1.

6-Chloro-N-(4-methoxyphenyl)-5methylpyrimidin-4-amine. Synthesized according to ihe method of reagent preparation 49 using 6-chloro-5-methylpyrimidin-4-ol and 4-methoxyaniline in step 1.

6-ChlbroT5-methyl-N-phenylpyrimidin-4-amine. Synthesized according to the method of reagent preparation.49 using 6-chloro-5-methylpyrimidin-4-ol and aniline in step 1.

### Reagent Preparation 50:5-(4,4,5,5-tetramethyl-1,3,2-dioxaborolan-2-yl)-1-methyl-1H-indazole

STEP 1: A suspension of 5-bromo-1 H-indazole (200 mg, 1.02 mmol), cesium carbonate (661 mg, 2.00 mmol), and iodomethane (156 mg, 1.10 mmol) in dimethylformamide (3 mL) was stirred at room temperature for 15 h. The mixture was partitioned between 5% lithium chloride and ethyl acetate, the aqueous layer was extracted with ethyl acetate (2 x), the combined organic extracts were washed with 1 N sodium hydroxide, and brine, dried over anhydrous sodium sulfate, filtered and concentrated. Column chromatography on silica (hexanes/ethyl acetate 4:1) gave 5-bromo-1-methyl-7H-indazole (150 mg, 70% yield) as an orange solid. MS (EI) for C₈H₇BrN₂: 212 (MH⁺).

STEP 2: A suspension of 5-bromo-1-methyl-1H-indazole (150 mg, 0.71 mmol). bis(pinacolato)diboron (200 mg, 0.78 mmol), potassium acctate (206 mg, 2.10 m mol), and dichlorol[1,1-bis(diphenylphosphino]ferrocenepalladium(II) dichloromethane adduct (36 mg, 0.04 mmol) in dimethyl sulfoxide (4 mL) was degassed with nitrogen, and then stirred at 80° C. for 18 h. The reaction mixture was cooled to room temperature and partitioned between water and ethyl acetate. The mixture was filtered through celite and then the layers were separated. The aqueous layerwas extracted with ethyl acetate (2 x), the combined organic extracts were washed with brine, dried over anhydrous sodium sulfate, filtered and concentrated. Column chromatography on silica (hexanes/ethyl acetate 7:3) provided 5- (4,4,5,5-tetramethyl-1,3,2-dioxaborolan-2-yl)- 1-methy-1H-indazdole (58 mg, 86% yield) as a yellow oil. MS (EI) for C₁₄H₁₉BN₂O₂: 259 (MH⁺).

### Reagent Preparation 51: 1,1-dimethylethyl 7-bromo-9-methyl-2,3-dihydro-1,4-benzoxazepine-4(5H)-carboxylate

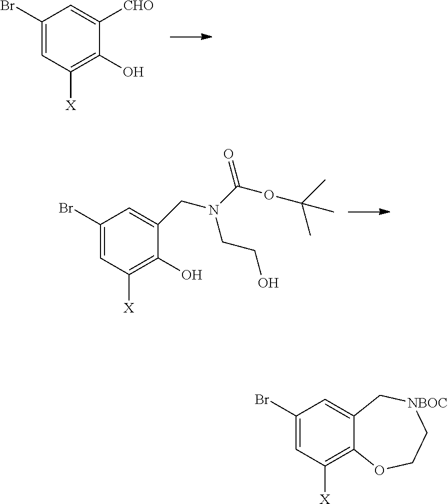

STEP 1: 5bromo-2-hydroxy-3-methylbenzaldehyde (64.6 g, 0.3 mol) was taken into a mixture of THF (80 mL) and methanol (80 mL) in a 1L3 neck flask equipped with a mechanical stirring apparatus and the mixture was gently warmed until a homogeneous solution was obtained. On cooling to room temperature, ethanolamine (23 mL, 0.38 mol) was added over 5 minutes. The resulting solution was stirred for 1 h al room temperature then cooled to 0° C. Sodium borohydride (4.26 g, 112 mmol) was added in portions followed by THF (65 mL) and the mixture was stirred for 1 h. Di-tert-bulyl dicarbonate (82 g, 0.375 mol) was then added asa concentrated solution in THF over 30 minutes. The resulting mixture was then allowed to warm to room temperature and and stirred an additioiial 2h. The mixture was concentrated to a thick residue and partitioned with ethyl acctateand water. The organic phases was washed twice with 1M aqueous hydrochloric acid once with water then brine dried over anhydrous sodium sulfate then filtered and concentrated. The residue was taken into a minimum of warm hcxanes and allowed to stand. The crystalline solid obtained was collected by filtration, washed with hexanes and dried to afford 1,1,-dimethylethyl [(5-bromo- 2-hydroxy-3-methylphenyl)](2-hydroxyethyl)carbamate (40 g). The mother liquor was concentrated and further purified by gradient silica gel chromatography using 3:1 hexanes:ethyl acetate to 100% ethyl acetate and the combined product fractions combined and concentrated. The residue was crystallized from a minimum of warm hexanes and combined with the previous crop to give 1,1-dimethylethyl [(5-bromo-2-hydroxy-3- methylphenyl)](2-hydroxyethyl)carbamate (61.87 g 57% yield) as a colorless crystalline solid.

STEP 2:1,1-dimethylethyl[(5-bromo-2-hydroxy-3-methylphenyl)](2- hydrbxyethyl)carbamate (10.0 g, 27.8 mmol) was taken into dichloromethane (50 ml) and the resulting solution cooled to 0° C. Diisopropylethylamine (5.8 mL, 33.4 mmol) was added to the solution followed by tosyl chloride (5.3 g, 27.8 mmol) and the mixture was allowed to warm to room temperature then stirred for 12h. The resulting slurry was concentrated and partitioned with ethyl ether and 1M aqueous hydrochloric acid. The organic solution was dried over sodium sulfate, filtered and concentrated to a colorless aniouphous residue. The residue obtained was taken into THF (50 mL) and cooled to 0° C. Sodium bis(trimethylsilyl)amide (5.3 g, 28.9 mmol) was added and stirring was continued for 1 h at which point additional sodium bis(trimethylsilyl)amide (5.3 g) was padded and the mixture was allowed to warm to room temperature and stirred for 12 h. The resulting slurry was partitioned with ethyl ether and 1M aqueous hydrochloric acid ahd the organic solution was dried over sodium sulfate, filtered and concentrated to a colorless amouphous residue. The residue was purified by silica gel chromatography to afford 1.1-dimethylethyl 7-bromo-9- methyl-2,3-dihydro-1,4-benzoxazepine-4(5H)-carboxylaic (6.9 g, 73 % yield) as a colorless oil that slowly crystallized. ¹HNMR (400 MHz, CDCI₃): 7.22 (s, 1.5H), 7.19 (s, 0.5H), 4.41 (br.s, 0.6H), 4.34 (br. s, 14H), 3:99 (m, 21-1). 3.79 (m, 2H), 2.20 (s, 3H). 1.4 (s. 9H); ¹HNMR (400 MHz, DMSO-d₆): 7.31 (br. s, 1H), 7.22 (br. s, 1H), 4.38 (br. s, 0.6H), 4.32 (s, 1.4H), 4.03 (m, 1H), 3.96 (m. 1H), 3.68 (m, 2H), 2,16 (s, 3H), 1.32 (s, 9H); MS (EI) for C₁₅H₂₀BrNO₃: 343 (MH⁺).

Proceeding according to the method of reagent preparation 1 and replacement of 5- bromo-2-hydroxy-3-methylbenzaldehyde in step 1 with alternative reagents, the following were prepared:

1,1-dimethylethyl 7-bromo-9-fluoro-2,3-dihydro-1,4-benzoxazepine-4(5H)- carboxylate. ¹H NMR (400 MHz, CDCI₃): 7.19 (m, 1.5H), 7.10 (s, 0.5H), 4.46 (br. s, 0.6H), 4,39 (br.s, 1.4H), 4.09 (m, 2H), 3.81 (m, 2H), 1.40 (s, 9H): ¹H NMR (400 MHz, DMSO-d₆): 7.50 (d, 1H), 7.27 (s, 1H), 4.45 (m, 2H), 4.11 (m, 2H), 3.92 (br.s, 2H), 1.29 (s, 9H); MS (EI) for C₁₄H₁₇BrFNO₃: 290 (M⁺-BOC).

1,1-dimethyleihyl 7-bromo-9-chloro-2,3idihydro-1,4-benzoxazepine-4(5H)- carboxylate. ¹NMR (400 MHz, CDC₃) δ 7.43 (d, 1H), 7.26 (d, 1H), 4.40 (s, 2H), 4.10 (m, 2H), 3.82 (m, 2H), 1.42 (s, 9H): MS (ES) for C₁₄H₁₇BrClNO₃: 362 (MH⁺).

1,1-dimethylethyl 7-bromo-9-ethyl-2,3-dihydro-1,4-benzoxazepine-4(5H)- carboxylate. MS (ES) for C₁₆H₂₂BrNO₃: 356, 358 (MH⁺).

1,1-dimethylelhyl 7-bromo-9-metthyloxy-2,3-dihydro-1,4-benzoxazepine-4(5H)- carboxylale. ¹H NMR (400 MHz, CDCl₃): 7.06-6.94 (m, 2H), 4.44 (bs, 2H), 4.04 (dd, 2H). 3.84 (s, 3H), 3.82-3.78 (m, 2H), 1.42 (s, 9H).

### Reagent Preparation 52: 4-Cldoro-5-isopropyl-6-methylpyrimidin-2-amine

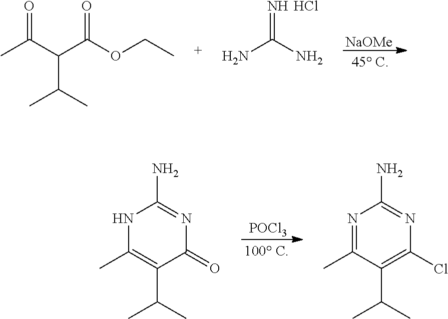

STEP 1: To a solution of ethyl 2-isopropylaectoacetate (22.0 g, 0.18 mol) and guanidine hydrochloiide (18.0 g, 0.19 mol) in methanol (100mL) was added sodium methoxide (0.38mol, 86.4 mL, 25 % methanol solution) at 0° C. via dropping funnel over 30 min. The reaction mixture was allowed to room temperature, then heated to 50° C. for 18 hrs. The mixture was concentrated, diluted with ethyl acetate (20 mL) and adjusted to pH 6-7 with 6N aqueous hydrochloric acid. The resulting solid was filtered and washed with water. The filtrates were concentrated and repeated filtration afforded a second crop of solid. The combined solids were dried under vacuum to give 2-amniino-5-isopropyl-6-methylpyrimidin- 4(1H)-one as a pale yellow solid (16.8 g, 56 %); ¹H NMR (400 MHz, DMSO-d₆): δ 10.5 (s, 1H), 6.17 (s, 2H), 2.85 (m, 1H), 2.03 (s, 3H), 1.15 (d, 6H); MS (EI) for C₈H₁₃N₃O: 168.2 (MH⁺).

STEP 2: To a solution of 2-amino-5-isopropyl-6-methylpyrimidin-4(1H)-one (4.93 g, 29.5 mmol) in phosphorus oxychloride (50 mL) was refluxed for 18 hrs. The reaction mixture was concentrated and the residue partitioned with a mixture of ethyl acetate and water (10 mL each). The biphasic mixture was quenched with solid sodium bicarbonate addition until the aqueous phase pHwas 6-7. The aqueous layer was extracted with ethyl acetate (3×100 mL) and the combined organic solutions dried over magnesium sulfate, filtered and concentrated to afford 4-chloro-5-isopropyl-6-methylpyrimidin-2-amine as a pale brown solid (4.92 g, 90%): ¹H NMR (400 MHz, DMSO-d₆): δ 6.71 (s, 2H), 3.26 (m, 1H), 3.25(s, 3H), 1.21 (d, 6H): MS(EI) for C₈H₁₂ClN₃: 186.1 (MH⁺).

Proceeding according to the method of reagent preparation 2 and replacing ethyl 2-isopropylacetoacetate in step 1 with alternative reagents, the following were prepared:

4-Chloro-5,6-dimethylpyrimidin-2-amine. ¹H NMR (400 MHz, DMSO-d₆): δ 6.69 (br s, 2H), 2.27 (s, 3H), 2.10 (s, 3H): MS (EI) for C₆H₈N₃Cl: 58.2 (MH^{+l ).}

4-Chloro-5-(2-methyoxyethyl)-6-methylpyrimidine-2-amine. MS (EI) for C₈H₁₂N_{3OCI:} 202.1 (MH⁺).

4-Chloro-6-ethyl-5-isopropylpyrimidin-2-amine. ¹H NMR (400 MHz, CDCl₃): δ 4.98 (br s, 2H), 3.42-3.26 (m, 1H), 2.72 (q, 2H), 1,34 (d, 6M), 1.27 (t, 3H),

4-Chloro-5-ethyl-6-methylpyrimidin-2-amine. ¹H NMR (400 MHz, DMSO-d₆): δ 6.73 (br s,2H), 2.60-2.47 (m, 2H), 2.30 (s, 3H), 1.04 (t, 3H): MS (EI) for C₇HₘN₃CI: 172.1 (MH⁺).

4-Chloro-5-isopropylpyrimidin-2-amine. MS (EI) for C₇H₁₀ClN₃: 172.1 (M⁺).

4-Chloro-6-methyl-5-propylpyrimidin-2-aimine. ¹H NMR (400 MHz, DMSO-d₆): 6.82 (s, 2H), 2.54-2.48 (m, 2H, overlapped), 2.32 (s, 3H) 1.56-1.37 (m, 2H), 0.93 (dt, 3H); MS (EI) for C₈H₁₂ClN₃: 186.1 (MH⁺).

4-Chloro-5-(cyclopropyylmethyl)-6-methylpyrimidin-2-amine. ¹H NMR (400 MHz, DMSO₆): 4.03 (br s, 2H), 2.55 (d, 2H), 2.35 (s, 3H), 0.99-0.88 (m, 1H), 0.49-0.34 (m, 2H), 0.22 (m, 2H); MS (EI) for C₉H₁₂ClN₃: 198.1 (MH⁺).

4-Chloro-6,6-dimethyl-5,6,7,8-tetrahydroquinazolin-2-amine. MS (EI) for C₁₀H₁₄ClN₃: 212 (M⁺).

5-Allyl-4-chloro-6-methylpyrimidin-2-amine. MS (EI) for C₈H₁₀ClN₃: 184 (MH⁺).

4,6-Dichloro-5-ethylpyrimidin-2-amine. ¹H NMR (400 MHz, DMSO-d₆): 11.11 (s, 1H), 7.33 (s, 1H), 6.71 (s, 2H), 2.62 (q, 2H), 2.33 (q, 2H), 1.07 (t, 3H), 0.96(t, 3H).

### Reagent Preparation 53: 1-(4-Chloro-5-isopropyl-6-methylpyrimidin-2-yl)-N,N,-dimethylmethanamine

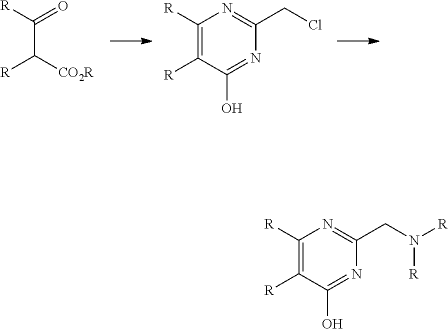

STEP 1: A pressure vessel was charged with methyl acetoacetate (40.0 g, 34.4 mmol), potassium carbonate (480 g, 34.7 mmol), and THF (200 mL). The heterogeneous mixture was slirred at rt for 45 min before adding 2-iodopropane (36.6 mL, 36.6 mmol), sealed and heated to 80° C. for 7-2 h with mixing. The reaction was then cooled to rt. Water was added in portions until all solid was dissolved to afford a homogeneous biphasic mixture. The mixture was partitioned, and the organic layer was/dried over sodium sulfate, filtered and concentrated under vacuum to afford a yellow oil. Distillation under vacuum afforded methyl 2-acetyl-3-methylbutanoate as a clear colorless oil. (20.0 g. 8:2 mix of methyl 2-acetyl-3- methylbutanoate: methyl aceloacetate. ¹H NMR (400 MHz, CDCI₃): δ 3.73 (s, 3H), 3.20 (d, 1H), 2.50-2.35 (m, 1H), 2.22 (s, 3H), 0.95 (dd, 6H); MS (EI) for C₈H₁₁O₃: 159.2 (MH⁺).

STEP 2: A round bottom flask was charged with methyl 2-aectyl-3- methylbutanoate as obtainedt in step 1 (14.3 g, 72.4 mmol), methanol (30 ), and 2- chloroacetamidine hydrochloride (12.8g, 99.5 mmol). The mixture was cooled to 0° C. followed by addition of 25 wt % sodium methoxide in methanol (48.8 mL, 181 mmol, 2.5 eq.). The reaction was warmed to rt. allowed to stir overnight, then filtered. The filter cake was rinsed with ethyl acetate and the organic solutions were combined, concentrated to a slurry, and the residue was purified by gradient silica gel chromatography (60:40 hexanes:ethyl acetate to 1:1 hexanes:ethyl acetate) to afford pure 2-(chloromethyl)-5-isopropyl-6- methylpyrimidin-4-ol as a yellow solid (3.17 g, 22 % yield). ¹H NMR (400 MHz, CDCl₃): δ 4.43 (s, 2H), 3.18-3.00 (m, 1H), 2.34 (s, 3H), 1.33 (d, 6H); MS (EI) for C₉H₁₃N₂OCl: 201.1 (MH⁺).

STEP 3:. A round-bottom flask was charged with 2-(chloromethyl)-5-isopropyl-6- methylpyrimidin-4-ol (500 mg, 2.5 mmol), THF (7 mL), and 2.0M dimethylamine in THF (2.5 mL, 5.0 mmol). The reaction was heated to 60° C. overnight, cooled to rt and concentrated under vacuum to afford crude 2-[(dimethylamino)methyf]-5-isopropyl-6- methylpyrimidin-4-ol as a brown oil. Neat phosphorous oxychloride (3 mL) was added and heated to 60° C. for 2 h. The react ion was cooled to rt, and concentrated under, vacuum. Ice cold water was added to the residue and then basified with 6N aqueous sodiumni hydroxide to pH 7. The aqueous mixture was extracted four times with ethyl acetate. The organic layers were combined dried over sodium sulfate, filtered and concentrated. The residue was purified by silica gel plug filtration, eluting with 95:5 ethyl acetate:methanol, then 90:10 ethyl acetate:methanol to afford pure 1-(4-chloro-5-isopropyl-6-methylpyrimidin-2-yl)-N,N- dimethylmethanamine as a brown oil (459 mg, 80 % yield). ¹H NMR (400 MHz, DMSO-d₆): δ 4.52 (s, 2H), 3.61-3.46 (m, 1H), 2.89 (s, 6H), 2.65 (s, 3H), 1.35 (d, 611): MS (EI) for C₁₁H₁₈N₃CI: 228.2 (MH^{+).}

Proceeding according to the method of Reagent Preparation 3 and isolating the second eluting compound in step 2 to afford 2-(methoxymethyl)-5-isopropyl-6- methylpyrimidin-4-ol, then procecding with step 3,4-chloro-5-isopropyl-2-(methoxymethyl)- 6-methylpyrimidine was prepared.

Proceeding according to the method of Reagent Preparation 3 and replacing methyl 2-acetyl-3-methylbutanoate in step 2 with alternative reagents, the following were prepared:

1-(4,6-dichloro-5-isopropylpyriinidin-2-yl)-N,N-dimethylmethanamine MS (EI) for C₁₀H₁₅N₃Cl₂: 248.1 (MH⁺).

1-(4-Chloro-5-isopropylpyrimidin-2-yl)-N,N-diimethymethanamine. ¹H NMR (400 MHz, CDCl₃) δ 8.59 (s, 1H), 3.87 (s, 2H), 3.38-3.19 (m, 1H), 2.52 (s, 6H), 1.40-1.23 (m, 6H): MS (EI) for C₁₀H₁₆ClN₃: 214, 216 (MH⁺, CI isotopes).

1 -(4-Chloro-5-ethylpyrimidin-2-yl)-N,N-dimethylmethanamine. ¹H NMR (400 MHz, CDCl₃) δ 8.52 (s, 1H), 3.70 (s, 2H), 2.74 (q, 2H); 2.37 (s, 6H), 1.28 (t, 3H); MS (EI) for C₉H₁₄ClN₃: 200, 202 (MH⁺, CI isotopes);

1-(4-Chloro-5,6-diethylpyrimidin-2-yl)-N,N-dimethylmethanamine. ¹H NMR (400 MHz, CDCl₃) δ 3.65 (s, 2H), 2.84 (q, 2H), 2.77 (q, 2H), 2.36 (s, 6H), 1.29 (t, 3H), 1.20 (t, 3H): MS (EI) for C₁₁H₁₈ClN₃: 228, 230 (MH⁺, CI isotopes).

1-(4-Chloro-6-ethyl-5-isopropylpyrimidin-2-yl)-N,N-dimethylmethanamine. ¹H NMR (400 MHz, DMSO-d₆): 3.68 (s, 2H), 3.48 (dt, 1H), 2.88 (q, 2H), 2.33 (s, 6H), 1.35 (d, 6H), 1.20 (q, 3H): MS (EI) for C₁₂H₂₀ClN₃: 242.1 (MH⁺).

1-(4-Chloro-5-(2,2,2-trifluoroethyl)pyrimidin-2-yl)-N,N-dimethylmethanamine. MS (EI) for C₉H₁₁ClF₃N₃: 254 (MH⁺).

1-(4,6-Dichloro-5-ethylpyrimidin-2-yl)-N,N-dimethylmethanamine. ¹H NMR (400 MHz, DMSO-d₆): 3.58 (s, 2H), 2.82 (q, 2H), 2.23 (s, 6H), 1.16 (t, 3H); MS (EI) for C₉H₁₃Cl₂N₄: 234 (MH⁺).

1-(4-Chloro-5-ethyl-6-methylpyrimidin-2-yl)-N,N-dimethylmethanamine. MS (EI) for C₁₀H₁₆ClN₃: 214.1 (MH⁺)

1-(4-Chloro-6-isopropyl-5-methylpyrimidin-2-yl)-N,N-dimethylmethanamine MS (EI) for C₁₁H₁₈ClN₃: 228.(H⁺)

Proceeding according to the method of Reagent Preparation 3 and replacing dimethylamine in step 3 with alternative reagents, the following were prepared:

4-Chloro-2-((3,3-difluoropyrrolidin-1-yl)methyl)-5-isopropyl-6-methylpyrimidine MS (EI) for C₁₃H₁₈N₃ClF₂: 272.2 (MH⁺).

4-Chloro-5-isopropyl-6methyl-2-[(4-methylpiperazin-1-yl)methyl]pyrimidine. ¹H NMR (400 MHz, CDCI₃) δ 3.72 (s, 2H), 3.58-3.44 (m, 1H), 2.78-2.34 (m, 11H), 2.28 (s, 3H), 1.37 (d, 6H); MS (EI) for C₁₄H₂₃ClN₄: 283 285 (MH⁺, CI isotopes).

4-[(4-Chloro-5-isopropyl-6-methylpyrimidin-2-yl)methyl]morpholine. MS (EI) for C₁₃H₂₀ClN₃O: 270.0 (MH⁺).

4-Chloro-5-isopropyl-6-methyl-2-(pyrrolidin-1-ylmethyl)pyrimidine. MS (EI) for C₁₃H₂₀ClN₃: 254 (MH⁺).

N-[(4-Chloro-5-isoproypl-6-methylpyrimidin-2-yl)methyl]-N-ethylethanamine. MS (EI) for C₁₃H₂₂ClN₃: 256 (MH⁺).

N-[(4-Chloro-5-ispropyl-6-methylpyrimidin-2-yl)methyl]-2-methylpropan-2-amine. ¹H NMR (400 MHz, DMSO-d₆): 3.76 (s, 2H), 3.30-3.24 (m, 1H), 2.57 (s, 3H), 1.32 (d, 6H), 1.06 (s, 9H); MS (EI) for C₁₃H₂₂ClN₃: 256,(MH⁺).

Proceeding according to the method of Reagent Preparation 3 and replacing methyl 2-acetyl-3-methylbutanoate in step 2 and dimeehylainine in siep 3 with alternative reagents, the following were prepared:

4-Chloro-5-isopropyl-2-(pyrrolidin-1ylmethyl)pyrimidine. ¹H NMR (400 MHz, CDCI₃) δ 8.56 (s, 1H), 3,88 (s, 2H), 3.40-3.15 (m, 1H), 2.82-2.54 (m, 4H), 1.99-1.79 (m, 4H), 1.31 (d, 6H); MS (EI) for C₁₂H₁₈ClN₃: 240, 242 (MH⁺, CI isotopes).

Proceeding according to the method of Reagent Preparation 3 and replacing methyl 2-acetyl-3-methylbutanoate and 2-chloroacetamidine hydrochloride in step 2 with alternative reagents, the following were prepared:

4-Chloro-2,6,6-trimethyl-5,6,7,8-tetrahydroquinazoline. MS (EI) for C₁₁H₁₅ClN₂: 211 (MH⁺).

### Reagent Preparation 54: N-(5-Bromo-2-chloropyridin-3-yl)methanesulfonamide

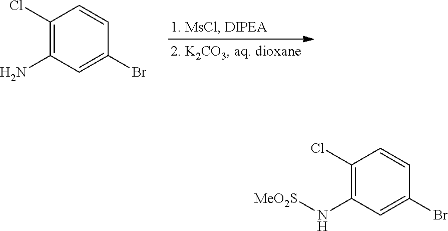 [SMILES: Nc1cc(Br)ccc1Cl ; CS(=O)(=O)Nc1cc(Br)ccc1Cl]

STEP 1: A solution of 5-bromo-2-chloropyridin-3-amine (1.0 g, 4.8 mmol) and diisopropylethylamine (1.85 mL, 10.6 mmol) in dichloromethane (25 mL) was cooled to 0° C., and then methanesulfonyl chloride (750 μL, 9.6mmol) was added slowly. The reaction mixture was stirred at 0° C. for 15 min and was then warmed to rt. After stirring for 2 h, water was added, and the biphasic mixture was partitioned. The organic phase was dried over magnesium sulfate, filtered, and concentrated in vacuo. The residue was then dissolved in dioxane (10 mL) and water (10 mL). Potassium carbonate (2.76 g, 20 mmol) was added, and the reaction, mixture was stirrced for 15 h at rt. Water was then added to the mixture which was subsequently acidified with aqueous citric acid (10%). The aqueous mixture was extracted twice with ethyl acetate. The combined organic extracts were dried oyer magnesium sulfate, filtered, and concentrated in vacuo. The residue was purified by flash chromatography (gradient, 100% hexanes to 50% hexancs in ethyl acetate) to provide N-(5- bromo-2-chloropyridin-3-yl)methanesulfonamide. (520 mg, 1.82 mnol, 38% yield) as a light pink solid. ¹H NMR (400 MHz, CDCI₃) δ 8.27 (d, 1H), 8.14 (d, 1H), 6.83 (br s, 1H), 3.11 (s, 3H): MS (EI) for C₆H₆BrClN₂O₂S: 285, 287, 289 (Br, Cl isotope pattern. MH⁺).

Proceeding according to the method of Reagent Preparation 4 and replacing Methanesulfonyl chloride with trifluoromethanesulfonic anhydride, the following was prepared:

N-(5-Bromo-2-chloropyridin-3-yl)-1.1,1 -trifluoromethanesulfonamide. MS (EI) for C₆H₃BrClF₃N₂O₂S: 338.9 (MH⁺).

### Reagent Preparation 55: 4-Chloro-6-methyl-5-vinylpyrimidin-2-amine

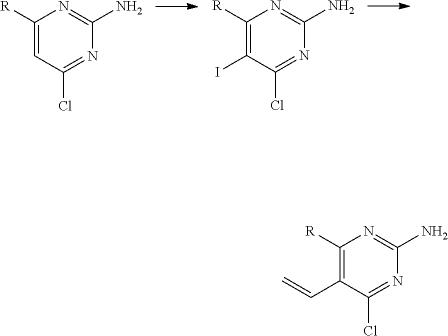

STEP 1: To a 50 mL pressure vessel were added 4-chloro-5-iodo-6- methylpyrimidin-2-amine (2.0 g, 7.43 mmol), 4,4,5,5-tetramethyl-2-vinyl-1,3,2- dioxaborolane (1.37 g, 8.17 mmol), dichloro[1,1 -bis(diphenyl)phosphinol]ferrocenepalladium (II) dichloromethane adduct (285 mg, 0.37 mmol, 5 mol %) and 2M sodium carbonate solution (7 mL) and 1,2-dimethoxyethane (20 mL). The reaction mixture was purged with nitrogen for 5 minutes and heated to 95° C. for 12 hours. The reaction was then cooled to room temperature and filtered through a pad of silica gel using ethyl acetate and the eluent concentrated. The residue was purified by gradient silica gel chromatography (hexanes:ethyl acetate 80:20 to 70:30) to afford 628 mg of 4-chloro-6-methyl-5-vinylpyrimidin-2-amine (53% yield), ¹H NMR (400 MHz, CDCl₃):6.60 (dd, 1H), 5.58 (dd, 1H), 5.47 (dd, 1H), 5.04 (s, 211), 2.44 (s, 3H): MS (EI) for C₇H₈ClN₃: 170.0 (MH⁺).

Proceeding according to the method of reagent preparation 5 and replacing 4- chloro-5-iodo-6-methylpyrimidin-2-amine with alternatiye reagents, the following were prepared:

4,6-dichloro-5-vinylpyrimidin-2-amine. ¹H NMR (400 MHz, DMSO-d₆): δ 7.59 (br m, 2H), 6.53 (dd, 1H), 5.66 (dd, 2H).

4-Chloro-5-vinylpyrimidin-2-a.ine. ¹H NMR (400 MHz, DMSO-d₆): 8.57 (s, 1H). 7.25 (s, 3H), 6.66 (dd, 1H), 5.77(d, 1H), 5.23 (d, 1H): MS (EI) for C₆H₆ClN₃: 156.1 (MH⁺).

Proceeding according to the method of reagent preparation 5 and replacing 4,4,5,5-tetramethyl-2-vinyl-1,3,2-dioxaborolane with alternative reagents, the following were prepared:

4-Chloro-5-3-fluorophenyl)-6-methylpyrimidin-2-amine. ¹H NMR (400 MHz, DMSO-d₆) δ 7.54-7.43 (m, 1H), 7.28-7.01 (m, 5H), 2.02 (sm 3H): MS (EI) for C₁₁H₉N₃FCI: 238.1 (MH⁺).

### Reagent Preparation 56: (S)-4-Chloro-7-methyl-5,67,8-tetrahydroquinazoline

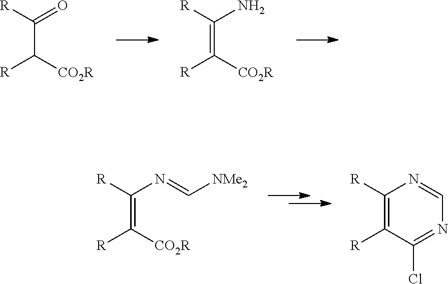

STEP 1: To a cooled (0° C.) solution of (S)-3-methylcyclohexanone (US20060293364)(2.0 g, 18 mmol) and dimethyl carbonate (2.0 mL, 22 mmol) in diethylether (40 mL) was added sodium hydride (60% wt/wt in mineral oil, 1.0 g, 25 mmol) portionwise over 30 minutes. The resulting slurry was allowed to stir at ambient temperature for 30 minutes followed by two hours at reflux. The reaction mixture was cooled (0° C.) and methanol (30 mL) was added dropwise over 20 minutes. The resulting slurry was partitioned between 10% aqueous citric acid and ethyl acetate. The organic layer was washed with brine, dricdover magnesium sulfate and concentrated in vacuo. Purification by silica gel column chromatography provided (4S)-methyl 4-methyl-2-oxocyclohexanecarboxylate (3.0 g, 100% yield). MS (ES) for C₉H₁₄O₃: 171 (M⁺).

STEP 2: A solution of (4S)-methyl 4-methyl-2-oxocyclohexanecarboxylate (3.0 g, 18 mmol) and ammonium acetate (3.4 g, 45 mmol) in ethanol (50 mL) was heated to reflux for 2 hours. The reaction was concentrated to one third original volume, and then diluted with ethyl-acetate (100 mL). The organic solution was washed with water (100 mL) and brine (50 mL) and then dried over anhydrous sodium sulfate. After filtration and concentration, the residue was dissolved in N,N-dimethylformamide dimethylacetal (50 mL) and heated to 110° C. for 18 hours. The resulting solution was cooled to room temperature and concentrated to provide (S.Z)-methyl 2-((dimethylamino)methyleneamino)-4- methylcyclohex-1-enecarboxylate (3.0 g, 88% yield) as an oil. MS (El) for C₁₂H₂₀N₂O₂: 224 (M⁺).

STEP 3: A solution of (S,Z)-melhyli 2-((dimethylamino)methyleneamino-4- methylcyclohex-1-enecarboxylate (3.5 g, 16 mmol) in 7.0M ammonia in methanol (35 mL) was stirred at 25° C. for 90 minutes then concentrated. The resulting oil was dissolved in chloroform (5 mL) and phosphorus oxychloride (5 mL) and refluxed for 2 hours. The mixture was concentrated to an oil, diluted with ethyl acetate (50 mL) and washed with saturated sodium carbonate (50 mL) and brine (25 mL). The solution was dried over anhydrous sodium sulfate, filtered and concentrated. The residue was purified by silica gel column chromatography (ethyl acetate:hexanes, 1:8) to give (S)-4-chloro-7-methyl-5,6,7,8- tetrahydroquinazoline (0.25 g, 8% yield) as a yellow oil. MS (ES): for C₉H₁₁ClN₂: 1.83 (MH⁺).

Using analogous synthetic techniques and substituting (S)-3-methyleyclohexanone with 4,4-dimethylcylohex-2-enone in step 1. 4-chloro-6,6-dimethyl-5,6-dihydroquinazoline was prepared. MS (ES) for C₁₀H₁₁ClN₂: 195 (MH⁺).

### Reagent Preparation 57: 4-Chloro-5-isopropyl-6-methylpyrimidine

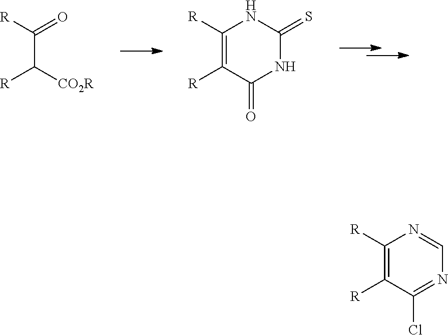

STEP 1: 5-isopropyl-2-mercato-6-methylpyrimidin-4-ol (0.37 g, 2.01 mmol, example 5, step 1) was added in portions into a mixture of 12% aqueous hydrogen peroxide (6 mL) and tetrahydrofuran (5 mL) al 70° C. and the resulting solution was stilted at this temperature for 30 min. After coolingio room temperature the pH was adjusted to 9 with saturated aqueous sodium carbonate, and the resulting mixture was stirred at room temperature for 30 min. A 10% aqueous solution of sodium thiosulfate was added until the reaction with iodine-starch paper was negative. The mixture was extracted with etltyl acetate (3×50 mL), and the combined organic layers were washed with brine (50 mL), dried over sodium sulfate, filtered and concentrated to provide 5-isopropyl-6-methylpyrimidin-4-ol (0.32 g, quantitative yield) as a colorless solid. MS (EI) for C₈H₁₂N₂O: 153 (MH⁺).

STEP 2: A solution of 5-isopropyl-6-methylpyrimidin-4-ol (0.32 g, 2.01 mmol) in phosphorus oxychiloride (5 mL) was stirred at 60° C. for 2 h. The reaction mixture was concentrated, ethyl acetate (10 mL) and saturated sodium bicarbonate (10 mL) was added to the residue, and the mixture was stirred at room temperature for 1 h. More ethyl acetate (50 mL) was added, the layers were separated, and the organic layer was washed with saturated sodium bicarbonate (5 mL), and brine (5 mL), dried over sodium sulfate, filtered and concentrated. Column chromatography of the residue on silica (0-30% ethyl acetate in hexanes) afforded 4-Chloro-5-isopropyl-6-methylpyrimidine (46 mg, 13% yield) as a colorless oil. MS (EI) for C₈H₁₁ClN₂: 171 (M⁺).

### Reagent Preparation 58: 1-[4-(7-Bromo-9-methyl-2,3-dibydro-1,4-benzoxazepin-4(5H)- yl)-5-isopropyo-6-methylprimidin-2-yl]-2,2,2-trifluoroethanol

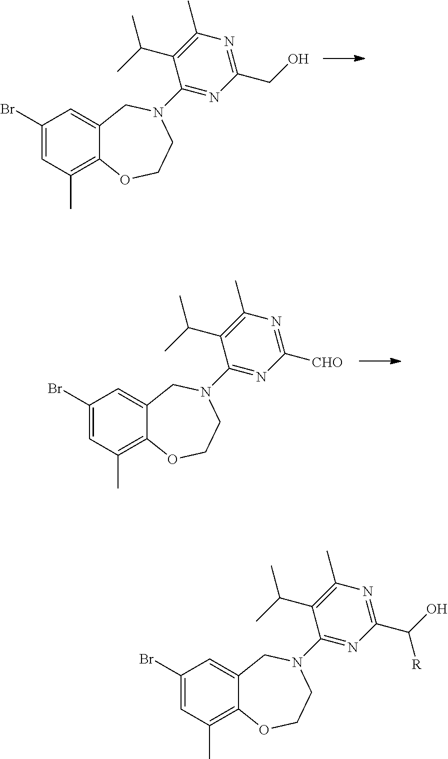

STEP 1: Dess-Martin periodinane (0.3l g, 0.74 mmol) was added to a cooled solution of [4-(7-bromo-9-methyl-2,3-dihydro-1,4-benzoxazepin-4(5H)-yl)-5-isopropyl-6- methylprrimidin-2-ylmethanol (0.20 g 0.49 mmol) and chloroform (10mL). The reaction mixture was allowed to warm to ambient temperature over 1 hour and was partitioned between saturated aqueous sodium bicarbonate and dichloromethane. The organic phase was then washed with brine, dried over magnesium sulfate, filtered and concentrated in vacuo. Purification by column chromatography provided 4-(7-bromo-9-methyl-2,3-dihydro-1,4- benzoxazepin-4(5H)-yl)-5-isopropyl-6-methylpyrimidine-2-carbaldehyde (0.15 g, 75% yield) as a waxy solid. MS (ES) C₁₉H₂₂BrN₃O₂: 404, 406 (MH⁺).

STEP 2: To a cooled (0° C.) solution of 4-(7-bromo-9-methyl-2,3-dihydro-1,4- benxoxazepin-4(5H)-yl)-5-isopropyl-6-methyrpyrimidine-2-carbaldehyde (0.09 g, 0.20 mmol) and cesium carbonate (0.19 g, 0.57 mmol) in THF (5 mL) was added trifluoromethyltrimethylsilane (0.08 mL, 0.54 mmol). The resulting mixture was stirred at ambient temperature for 24 hours and methanol (1 mL) was added. the resulting slurry was concentrated in vacuo and partitioned between dichloromethane and water. The organic layer was washed with brine, dried over MgSO₄, filtered and concentrated in vacuo. Purification by silica gel column chromatography provided 1-[4-(7-bromo-9-methyl-2,3-dihydro-1,4- benzoxazepin-4(5H)-yl)-5-isopropyl-6-methylpyrimidin-2yl]2,2,2-trifluoroethanol (25 mg, 28% yield) as a clear waxy solid. MS (ES) C₂₀H₂₃BrF₃N₃O₂: 474, 476 (MH⁺).

### Reagent Preparation 59: 1-[4-(7-Bromo-9-methyl-2,3-dihydro-1,4-benzoxazepin-4(5H)- yl)-5-isopropyl-6-methylpyrimidin-2-yl]ethanol

STEP 1: To a cooled (0° C.) solution of 4-(7-bromo-9-methyl-2,3-dihydro-1,4- benzoxasepin-4(5H)-yl)-5-isoprppyl-6-methylpyrimidine-2-carbaldeliyde (0.09 g, 0.20 mmol) in THF (5 mL) was added methytmagensium bromide (200 mL, 3.0 M solution in hexanes). The reaction mixture was allowed to stir at room temperature for 1 hour and saturated aqueous ammonium chloride (5 mL) was added. The resulting slurry was partitioned between dichloromethane and water and the organic layer was washed with brine, dried over magnesium sulfate, filtered and concentrated in vacuo. Purification of the residue by silica gel column chromatography provided 1-[4(7-bromo-9-methyl-2,3-dihydro-1,4- benzoxasepin-4(5H)-yl )-5-isopropyl-6-methylpyrimidin-2-yl]ethanol (30 mg, 38% yield). MS (ES) C₂₀H₂₆BrN₃O₂: 420, 427 (MH⁺).

### Example 1

### 6-{4-[2-Amino-6-methyl-5-(1-methylethyl)pyrimidin-4-yl]-9-methyl-2,3,4,5- tetrahydro-1,4-benzoxazepin-7-yl)}[1,3]thiazolo[5,4-b]pyridin-2-amine

STEP 1: To a 100 mL pressure vessel were added tert-butyl 7-bromo-9- methyl-2,3-dihydro-1,4-benzoxasepine-4(5H)-carboxylate (9.2 g, 0.026 mol), bispinacolato(diboron) (8.2 g, 0.032 mol) and potassium acetate (7.6 g, 0.078 mol) in dioxane (50 mL). Dichloro[1,1-bis(diphenyl)phosphino]ferrocenepalladium (II) dichloromethane adduct (530 mg, 0.65 mmol, 2.5 mol %) was added and nitrogen was bubbled through the reaction mixture for 5 minutes. The reaction mixture was heated to 95° C. for 12 hours, cooled to room temperature and filtered through a pad of Celite®. The Celite® pad was washed with ethyl acetate (2×100 mL) and the combined organic layers were dried over sodium sulfate. filtered, and concentrated. The residue was purified by gradient silica gel flash chromography (hexanes:ethyl acetate 80:20to 70:30) to afford tert-butyl 9-methyl-7- (4,4,5,5-tetramethyl-1,3,2-dioxaborolan-2-yl)-2,3-dihydro-1,4-benzoxazepine-4(5H)- carboxylate (10.31 g, quantitative yield). ¹H NMR (400 MHz, CDCI₃): δ 7.66-7.43 (m, 2H), 4.45 (d, H), 4.03 (s, 2H), 3.87-3.75 (m, 2H), 2.23 (s, 3H), 1.41 (s, 9H), 1.36-1.26 (s, 12H): MS (EI) for C₂₁H₃₂BNO₅: 288.2, 290.2 (MH⁺-Boc).

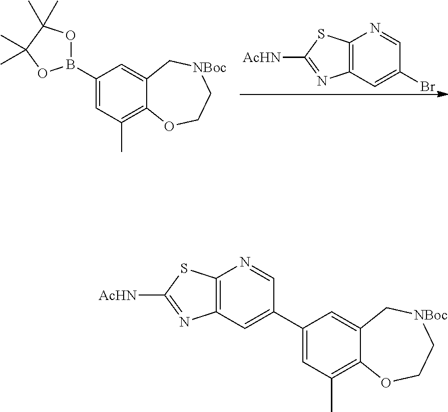 [SMILES: Cc1cc(B2OC(C)(C)C(C)(C)O2)cc2c1OCCN(C(=O)OC(C)(C)C)C2 ; CC(=O)Nc1nc2cc(Br)cnc2s1 ; CC(=O)Nc1nc2cc(-c3cc(C)c4c(c3)CN(C(=O)OC(C)(C)C)CCO4)cnc2s1]

STEP 2: A glass pressure vessel was charged with tert-butyl 9-methyl-7-(4,4,5,5- tetramethtyl-1,3,2-dioxaborolan-2-yl)-2,3-dihydrobenzol[1,4]oxazepine-4(5H)-carboxylate (12.0 g, 30.8 mmol), N-(6-bromothiazolo[5,4]pyridine-2-yl)acetamide (8.35 g, 30.8 mmol). DME (75 mL), and 2M Na₂CO₃(aq) (31.2 mL, 61.6 mmol). The reaction mixture was purged with nitrogen followed by addition of dichloro[1,1-bis- (diphenyl)phosphino]ferrocenepalladium (II) dichloromcthane adduct (1.27 g, 5 mol %) then heated to 80° C. for 23 h. The vessel was then cooled to rt, and the reaction mixture was filtered through Celite® and the filter cake rinsed with ethyl acetate. The combined organic filtrate was concentrated to a brown residue which was take into ethanol (30 mL) and ethyl acetate (10 mL and allowed to stir at rt for 1 h until a fine precipitate formed. The precipitate was collected by filtration, rinsed with ethanol and dried under vacuum to afford to pure product as a light tan solid (5.9g). The filtrate was concentrated and purified by silica gel flash chromatography (1:1 hexanes:ethyl acetate) to afford additional tert-butyl 7-(2- acetamidothiazolo[5,4-b]pyridin-6-yl)-9-methyl-2,3-dihydrobenzo[1,4]oxazepine-4(5H)- carboxylate (0.74 g) to give a combined yield of (6.67 g, 48% yield), ¹H NMR (400 MHz, DMSO-d₆): δ 12.56 (s, 1H), 8.73 (s, 1 H), 8.25 (m, 1H), 7.54 (m, 2H), 4.47 (m, 2H), 4.20- 3.97 (m, 2H)m 3.73 (s, 2H), 2.26 (s, 3H), 2.24 (s, 3H), 1.34 (s, 9H): MS (EI) for C₂₃H₂₆N₄O₄S: 555.1 (MH⁺).

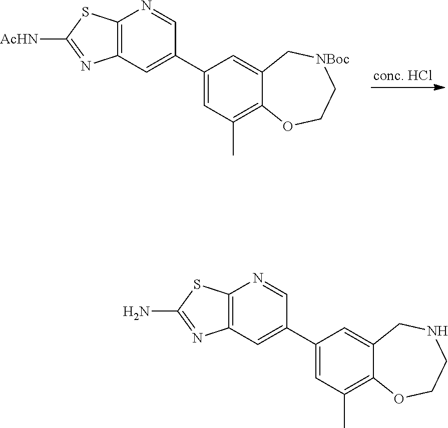 [SMILES: CC(=O)Nc1nc2cc(-c3cc(C)c4c(c3)CN(C(=O)OC(C)(C)C)CCO4)cnc2s1 ; Cc1cc(-c2cnc3sc(N)nc3c2)cc2c1OCCNC2]

STEP 3: A 500 mL round bottom flask was charged with tert-butyl 7-(2- acetamidothiazolo-[5,4-b]pyridin-6-yl)-9-methyl-2,3-dihyrobenzo[1,4]oxoazepine-4(5H)- carboxylate (6.67 g, 14.7 mmol), ethanol (8 mL), and concentrated aqueous hydrochloric acid (37 mL). The resulting slurry was stirred at ft for 20 min then heated to reflux overnight with stirring. The reaction mixture was then cooled to rt. and concentrated to 1/3 the volume. Acetonitrile (20 mL) was added to produce a fine precipitate that was collected by filtration, rinsed with acetonitrile and dried under vacuum to give pure 6-(9-methyl-2,3,4,5-tetrahydro- 1,4-benzoxazepin-7-yl)[1,3]thiazolo[5,4-b pyridin-2-amine hydrochloride salt (5.65 g, 14.7 mmol, quantitative yield). ¹H NMR (400 MHz, DMSO-d⁶): δ 9.67 (br s, 2H), 8.47 (m, 2H), 7.91 (d, 1H), 7.70 (dd, 2H), 4,36 (br s, 2H), 4.23 (br s, 2H), 3.49 (br s, 2H), 2.29 (s, 3H); MS (EI) C₁₆H₁₆N₄OS: 313.1 (MH⁺).

STEP 4: A mixture of 6-(9-methyl-2,3,4,5-tetrahydro-1.4-benzoxazepin-7- yl)[1,3-thiazolo[5,4-b]pyridin-2-amine dihydrochloride (0.26 g, 0.67 mmol), 4-chloro-5- isopropyl-6-methylpyrimidin-2-amine (0.12 g, 0.67 mmol) and N,N-diisopropylethylamine (0.6 mL, 3.35 mmol) in N,N-dimethylacetamide (3.0 mL) was healed at reflux for 30 minutes. Afler cooling to room temperature the reaction mixture was diluted with water (75 mL) and the precipitate thus formed was collected by filtration, washed with hexanes and driedin vacuo. Gradient silica gel chromatography (dichloromethane:methanol 95:5 to 85:15) provided 6-{4-[2-amino-6-methyl-5-(1-methylethyl)pyrimidin-4-yl]-9-methyl-2,3,4,5- tetrahydro-1,4)benzoxazepin-7-yl)ο[1,3[thiazolo[5,4-b]pyridin-2-amine (0.16 g, 52%). ¹H NMR (400 MHz, CD₃OD) δ 8.66 (d, 1H), 8.01 (d, 1H), 7.52 (d, 1H), 7.47 (d, 1H), 4.89 (s, 2H), 4.45 (m, 2H), 4.04 (m, 2H), 3.08 (m, 1 H). 2.44 (s, 3H), 2.30 (s, 3H), 1.36 (d, 6H): MS (ES) for C₂₄H₂₇N₇OS: 462 (MH⁺).

Proceeding according to the method of Example 1 and replacing 4-chloro-5 isopropyl-6-methylpyrimidin-2-amine in step 4 with alternative reagents, the following compounds of the invention were prepared:

b 4-(2-Amino-5,6-dimethylpyrimidin-4-yl)-9methyl-2,3,4,5-tetrahydro-1,4- benzoxazepin-7-yl][1,3]thiazolo[5,4-b]pyridin-2-amine. ¹H NMR (400 MHz, DMSO-d₆): δ 8.37 (d, 1H), 7.90-7.77 (m, 3H), 7.50 (dd, 2H), 6.51 (br s, 2H), 4.63 (s, 2H), 4.32 (m, 2H), 3.83 (m, 2H), 2.25 (s, 3H), 2.18 (s 3H), 2.05 (s, 3M); MS (EI) for C₂₂H₂₃N₇OS: 434.2 (MH⁺).

6-[4-(2-Amino-5-ethyl-6-methylpyrimdin-4-yl)-9-methyl-2,3,4,5-tetrahydro-1,4- benzoxazepin-7-yl][1,3]thiazolo[5,4-b]pyridin-2-amine. ¹H NMR (400 MHz, DMSO-d₆): δ 12.50 (s, 1H), 8.41 (s, 1H), 7.93 (s, 2H), 7.86 (s, 1H), 7.62 (s, 1H), 7.49 (s, 2H), 4.92 (s, 2H), 4.39 (s, 2H), 4.08 (s, 2H), 3.40 (buried q, 2H), 2.29 (s, 3H), 2.24 (s,3H), 1.08 (t,3H); MS (EI) for C₂₃H₂₅N₇OS: 448.2 (MH⁺).

6-[4-(2-Amino-5-ethenyl-6-methylpyrimidin-4-yl)-9-methyl-2,3,4,5-tetrahydro-1,4- benzoxazepin-7-yl][3]thiazolo[5,4-b]pyridin-2-amine. ¹H NMR (400 MHz, DMSO-d₆): 833 (d, 1H), 7.86 (s, 2H), 7.78 (d, 1H), 7.44 (s. 1H), 7.36 (s, 1H), 6.56 (dd, 1H), 6.12 (s, 2H), 5.37 (dd, 1H), 5.23 (dd, 1H),.4.66 (s, 2H), 4.23 (s, 2H), 3.83 (s, 2H), 2.24 (s, 3H), 2.19 (s, 3H), 1.90 (s, 3H, OAc); MS (EI) for C₂₃H₂₃N₇OS: 446.1 (MH⁺).

6-[4-[2-Amino-5-(1-methylethyl)pyrimidin-4-yl]-9-methyl-2,3,4,5-tetrahydro-1,4- benzoxazepin-7-yl][1,3]thiazolo[5,4-b]pyridin-2-amine. ¹H NMR (400 MHz, DMSO-d₆): 8.35 (d, 1H), 7.94 (s, 1H), 7.87 (s, 2H), 7.79 (d, 1H), 7.49 (s, 1H), 7.40 (d, 1H), 6.02 (d, 2H). 4.44 (s, 2H), 4.27 (s, 2H), 3.69 (s, 2H), 2.97 (dt, 1H), 2.26 (s, 3H), 1. 17 (d, 6H); MS (EI) for C₂₃H₂₅N₇OS: 448.2 (MH⁺).

4-Amino-2-[7-(2-amino[1,3]thiazolo[5,4-b]pyridin-6-yl)-9-methyl-2,3-dihydro-1,4- benzoxazepin-4(5H)-yl]pyrimidine-5-carbonitrile. ¹H NMR (400 MHz,. DMSO-d₆): 8.40 (d, 1H), 8.28 (d, 1H), 8.01 (s, 2H), 7.84 (d, 1H), 7.64 (s, 1H), 7.47 (s, 2H), 7.42-7.23 (m, 1H), 4.87 (m, 2H), 4.14 (t, 4H), 2.23 ( s, 3H): MS (EI) for C₂₁H₁₈N₈OS: 431.1 (MH⁺).

4-Amino-2-[7-(2-amino[1,3]thiazolo[5,4-b]pyridin-6-yl)-9-methyl-2,3-dihydro-1,4- benzoxazcpin-4(5H)-yl]pyrimidine-5-carboxamide. ¹H NMR (400 MHz, DMSO-d₆): δ 8.38 (d, 3H), 7.84 (d, 4H), 7.61 (s, 2H), 7.08-6.84 (m, 1H), 4,82 (s, 2H), 4.13 (d, 4H), 2.23 (s, 3H): MS (EI) for C₂₁H₂₀N₈O₂S: 449.1 (MH⁺).

6-[7-(2-Amino[1,3]thiazolo[5,4-b]pyridin-6-yl)-9-methyl-2,3-dihydro-4- benzoxazepin-4(5H)-yl]pyridine-3-carbonitrile. ¹H NMR (400 MHz. DMSO-d₆): 8.47 (d, 1H), 8.37 (d, 1H), 7,93-7.80 (m, 4H), 7.75 (s, 1H), 7.48 (s, 1H), 7.13 (d, 1H), 4.87 (s, 2H), 4.23 (s, 2H), 4.15 (s, 2H), 2.22 (s, 3H); MS (EI) for C₂₂H₁₈N₆OS: 415.1 (MH⁺).

6-[4-(4-Amino-5-methylpyrimidin-2-yl)-9-methyl-2,3,4,5-tetrahydro-1,4- benzoxazepin-7-yl][1,3]thiazolo[5,4-b]pyridin-2-amine. ¹H NMR (400 MHz, DMSO-d₆): 8.36(d, 1 H), 7.85 (s, 2H), 7.80 (d, 1H), 7.54 (s, 2H), 7.40 (d, 1H), 6.32 (s, 2H), 4.76 (s, 2H), 4.05 (d, 4H), 2.21 (s, 3H), 1.88 (s, 3H, OAc), 1.79 (s, 3H ); MS (EI) for C₂₁H₂₁N₇OS: 420.1 (MH⁺).

6-[4-(2-Amino)-6-methyl-5-propylpyimidin-4-yl)-9-methyl-2,3,4,5-tetrahydro-1,4- benzoxazepin-7-yl][1,3]thiazolo[5,4-b]pyridin-2-amine. ¹H NMR (400 MHz, DMSO-d₆): 8.36 (d, 1H), 7.85 (s, 2H), 7.81 (d, 1H), 7.47 (d, 2H), 5.92 (s, 2H),4.43 (s, 2H), 4.27 (s, 2H), 3.63 (s, 2H), 2.46-2.36 (m, 2H), 2.26 (s, 3H), 2.18 (s, 3H), 1.49-1.30 (m, 2H), 0.73 (t, 3H): MS (EI ) for C₂₄H₂₇N₇OS: 461.9 (MH⁺).

6-(4-[2-Amino-5-(cyclopropylmethyl)-6-methylpyrimidin-4-yl]-methyl-2,3,4,5- tetrahydro-1,4-benzoxazepin-7-yl][1,3]thiazolo[5,4-b]pyridin-2-amine. ¹H NMR (400 MHz, DMSO-d₆): 8.42 (d, 1H), 7.93 (s, 2H), 7.87 (d, 1H), 7.56 (d, 2H), 6.04 (s, 2H), 4.47 (s, 2H), 4.31 (s, 2H), 3.71 (s, 2H), 2.53 (d, 2H), 2.33 (s, 3H), 2.30 (s, 3H), 1.89 (d, 2H, OAc), 0.88 (s, 1H), 0.47-0.27 (m, 2H), 0.00 (q, 2H); MS (EI) for C₂₅H₂₇N₇OS: 473.9 (MH⁺).

6-(4-{2[(Dimethylamino)methyl]-6-ethyl-5-(1 -methylethyl)pyrimidin-4-yl}-9- methryl-2,3,4,5-tetrahydro-1,4-benzoxazepin-7-yl)[1,3]thiazolo[5,4-b]pyridin-2-amine. ¹H NMR (400 MHz, DMSO-d₆): 8.34 (d, 1H), 7.87 (s, 2H), 7.78 (d, 1H), 7.49(s, 1H), 7.41 (s, 1H), 4.39 (s, 2H), 4.29 (s, 2H), 3.67 (s, 3H), 2.80-2.67 (m, 2H), 2.53 (d, 1H), 2.26 (s, 3H), 2.16 (s, 6H), 1.31 (d, 6H), 1.20 (t, 3H): MS (EI) for C₂₈H₃₅N₇OS: 518.3 (MH⁺).

6-[4-(2-Amino-5-ethenylpyrimidin-4-yl)-9-methyl-2,3,4,5-tetrahydro-1,4- benzoxazepin-7-yl][1,3]thiazolo[5,4-b]pyridin-2-amine. ¹H NMR (400 MHz, DMSO-d₆): 8.34 (dd,1H), 7,88 (d, 1H), 7.86 (s, 2H), 7.77 (d, 1H), 7.46 (d, 1H), 7.37 (d, 1H), 6,56 (dd, 1H), 6.26 (s, 2H), 5.43 (dd, 1H), 5.08 (dd, 1H), 4.69 (s, 2H), 4.24 (d, 2H), 3.92 (d, 2H), 2.24 (s, 3H), 1.90 (d, 2H OAC); MS (EI) for C₂₂H₂₁N₇OS: 431.9 (MH⁺).

6-{9-Methyl-4-[6-methyl-5-(1-methylethyl)-2-(morpholin-4-ylmethyl)pyrimidin-4- yl]-2,3,4,5-tetrahydro-1,4-benzoxazepin-7-yl][1,3]thiazolo[5,4-b]pyridin-2-amine. ¹H NMR (400 MHz, DMSO-d₆): 8.34 (d, 1H), 7.88 (s, 2H), 7.77 (t,1H), 7.48 (d, 1H), 7.39 (d, 1H), 4.45 (s, 2H), 4.32 (s, 2H), 3.68 (s, 2H), 3.51-3.41 (m, 4H), 1.27 (dd, 2H), 2.51 (s, 1H). 2.46 (s, 4H), 2.3 (s, 3H), 2.22 (d, 3H), 1.91 (s, 2H, OAc), 1.31 (d, 6H); MS (EI) for C₂₉H₃₅N₇OS: 546.2 (MH⁺).

6-(4-{2-Amino-6-methyl-5-[2-(methyloxy)ethyl]pyrimidin-4-yl]-9-methyl-2,3,4,5- tetrahydro-1,4-benzoxazepin-7-yl)[1,3]thiazolo[5,4-b]pyridin-2-amine. ¹H NMR (400 MHz, DMSO-d₆): δ 8.38 (d, 1H), 7.93-7.79 (m, 3H), 7.50 (s, 2H), 6.03 (s, 2H), 4.39 (s, 2H), 4.24 (m, 2H), 3.64 (m, 2H), 3.45 (t. 2H), 3.20 (s, 3H), 2.75 (t, 2H), 2.27 (s, 3H), 2.21 (s, 3H), 2.07 (s, 21H-OAc peak); MS (EI) for C₂₄H₂₇N₇O₂S: 478.2 (MH⁺).

6-[4-(4-Aminopyridin-2-yl)-9-methyl-2,3,4,5-tetrahydro-1,4-benzoxazepin-7- yl][1,3]thiazolo[5,4-b]pyridin-2-amine. ¹H NMR (400 MHz, DMSO-d₆): δ 8.28 (d, 1H), 7.79 (s, 2H), 7.73 (d, 1H), 7.63 (d, 1H), 745 (s, 1H), 7.35 (d, 1H), 6,39 (s, 214), 5.61 (d, 1H), 4.72 (s, 2H), 4.01 (br d, 4H), 2.16 (s, 3H), 1.75 (s, 2H-OAc peak); MS (EI) for C₂₀H₁₉N₇OS: 406.1 (MH⁺).

3-[7-(2-Amino[1,3]thiazolo[5,4-b]pyridin-6-yl)-9-methyl-2,3-dihydro-4- benzoxazepin-4(5H)-yl]pyrazine-2-carbonitrile. ¹H NMR (400 MHz, DMS₆): δ 8.41 (d, 1H), 8.34 (d, 1H), 8.06 (d, 1H), 7.87 (s, 2H), 7.79 (d, 1H), 7.57 (s, 1H), 7.48 (s, 1H), 5.08 (s, 2H), 4.38 (m, 2H), 4.23 (m, 2H), 2.23 (s, 3H); MS (EI) for C₂₁H₁₇N₇S: 416.1 (MH⁺).

6-[4-(4-Amino-5-fluoropyrimidin-2-yl)-9-metyl-2,3,4,5-tetrabydro-1,4- benzoxazepin-7-yl][1.3]thiazolo[5.4-b]pyridin-2-amine. ¹H NMR (400 MHz, DMSO-d₆): δ 8.36 (d, 1H), 7.86 (s, 2H), 7.81 (d, 1H), 7.76 (d, 1H), 7.52 (s, 1H), 7.42 (d, 1H), 6.91 (br s, 2H), 4.75 (s, 2H), 4.06 (br s, 4H), 2.25 (s, 3H); MS (EI) for C₂₀H₁₈N₇OSF: 424.1 (MH⁺),

2-[7-(2-Amino[1,3]thiazolo[5,4-b]pyridin-6-yl)-9-methyl-2,3-dihydro-1,4- benzoxazepin-4(5H)-yl]pyridine-3-carbontrile. ¹H NMR (400 MHz, DMSO-d₆): δ 8.39- 8.29 (m, 2H), 8.03 (dd, 1H), 7.87 (s, 2H), 7.78 (d, 1H), 7.55 (d, 1H), 7.45 (d, 1H), 6.82 (dd, 1H), 5.06 (s, 2H), 4.32 (m, 2H), 4.17 (m, 2H), 2.23 (s, 3H): MS (EI) for C₂₂H₁₈N₆OS: 415.1 (MH⁺).

2[7-(2-Amino[1,3]thiazolo[5,4-b]pyridin-6-yl)-9-methyl-2,3-dihydro-1,4- benzoxazepin-4(5H)-yl]pyridine-3-carboxamide. ¹H NMR (400 MHz, DMSO-d₆): δ 8.33 (d, 1H), 8.88 (dd, 1H), 8,02 (s, 1H), 7.85 (s, 2H), 7.79 (d, 1H), 7.65-7.54 (m, 2H), 7.41 (d, 2H), 6.71 (dd, 1H), 4.76 (s, 2H), 4.29 (m, 2H), 3.84 (m, 2H), 2.21 (s, 3H); MS (EI) for C₂₂H₂₀N₆O₂S: 433.1 (MH⁺).

6-[4-(2-Amino-6-chloro-5-ethenylpyrimidin-4-yl)-9-methyl-2,3,4,5-tetrahydro-1,4- benzoxazepin-7-yl][1,3]thiazolo[5,4-b]pyridin-2-amine. ¹HH NMR (400 MHz, DMSO-d₆): δ 8.33 (d, 1H), 7.91-7.77 (m,3H), 7.41 (d, 2H), 6.6 (br s, 2H), 6.55 (dd, 1H), 5.40 (dd,2H), 4.71 (s, 2H), 4.29-4.11 (m, 2H), 3.75 (m, 2H), 2.22 (s, 3H): MS(EI) for C₂₂H₂₀N₇OSCI: 466.1 (MH⁺).

6- {4-(2-Amino-5-(3-fluorophenyl)-6-methylpyridin4-yl]-9-methyl-1-2,3,4,5- tetrahydro-1,4-benzoxazepin-7-yl][1,3]thiazolo[5,4-b]pyridin-2-amine. ¹H NMR (400 MHz, DMSO-d₆): δ 8.31 (d, 1H), 7,87 (s, 2H), 7.76 (s, 1H), 7.49-7.33 (m, 2H), 7.09 (d, 2H), 7.02 (t, 1H), 6.95 (s, 1H),6.18 (s, 2H), 4.34 (s, 2H), 4.06 (s, 2H), 2.20 (s, 3H), 1.86 (s, 3H); MS (EI) for C₂₇H₂₄N₇OSF: 514.1 (MH⁺),

6-Amino-2-[7-(2-amino[3]thiazolo[5,4-b]pyridin-6-yl)-9-methyl-2,3-dihydro-1,4- benzoxazepin-4(5H)-yl]pyridin-3-carbonitrile. ¹H NMR (400 MHz, DMSO-d₆): δ 8.34 (d, 1H), 7.86 (s, 2H), 7.79 (d, 1H), 7.59 (d, 1H), 7.48-7.38 (m, 2H), 6.69 (s, 2H), 5.81 (d, 1H), 4.87 (s, 2H), 4.20 (m, 2H), 4.11 (m, 2H); 2.24 (s. 3H): MS (EI) C₂₂H₁₉N₇OS: 430.1 (MH⁺).

6-{4-[2-Amino-6-ethyl-5-(1-methylethyl)pyrimidin-4-yl]-9-methyl-2,3,4,5- tetrahydro-1,4-benzoxazepin-7yl][1,3]thiazolo[5,4-b]pyridin-2-amine. ¹H NMR (400 MHz, DMSO-d₆): δ 8.34 (s, 1H), 7.87 (s, 2H), 7.78 (s, 1H), 7.51 (s, 1H), 7.38 (s, 1H), 6.02 (br s., 2H), 4.23 (br s,4H), 3.53 (br s, 2H), 3.22 (m, 1H), 2.58,(q, 2H), 2.26 (s, 3H), 1.91 (s, 2H- OAc peak), 1.31 (d, 6H), 1.16(t, 3H); MS (EI) for C₂₅H₂₉N₇OS: 476.2 (MH⁺)

6-{4-[2-Amino-6-chloro5-(1-methylethyl)pyrimidin-4-yl]-9-methyl-2,3,4,5- tetrahydro-1,4-benzoxazepin-7-yl][1,3]thiazolo[5,4-b]pyridin-2-amine. 1H NMR (400 MHz, DMSP-d₆): δ 8.45 (d, 1H), 7.55-7.48 (m, 2H), 7.33 (d, 1H), 6.54 (s,. 2H), 4.37 (s, 2H), 4.26 (m. 2H), 3.65 (m, 2H), 3.15-3.02 (m, 1H), 2.27 (s, 314), 1.30 (d, 6H); MS (EI) for C₂₃H₂₄N₇OSCI: 482.1 (MH⁺).

6-(4-{6-Chloro-2-](dimethylamino)methyl]-5-(1-methylethyl)pyrimidin-4-yl)-9- methyl-2,3,4,5-tetrahydro-1,4-benzoxasepin-7-yl)[1,3]thiazolo[5,4-b]pyrimdin-2-amine. ¹H NMR (400 MHz, DMSO-d₆); δ 8.33 (s, 1H), 7.85(s, 1H), 7.77 (s, 1H), 7.44 (d, 2H), 4.56 (s, p 2H), 4.31 (br s, 2H), 3.77 (br s, 2H); 3.25 (buried s, 2H), 3.23-3.04 (m, 1H), 2.21 (s, 3H), 2.07 (s, 6H), 1.35 (d, 6H): MS (EI) for C₂₆H₃₀N₇OSCl: 524.2 (MH⁺),

6-(4-{2-[(3,3-Difluoropyrolidin-1-yl)methyl]-6-methyl-5-(1-methylethyl)pyrimidin- 4-yl]-9-methyl-2,3,4,5-tetrahydro-1,4-benzoxazepin-7-yl)[1,3]thiazolo[5,4-b]pyridin-2- amine. ¹H NMR (400 MHz, DMSO-d₆): δ 8.32 (s, 1H), 7.84 (s, 2H), 7.75 (s, 1H), 7.45 (, 1H), 7.37 (s, 1,14), 4.42 (s, 2H), 4.30 (s, 2H), 3.66 (s, 2H), 3.52 (s, 2H), 3.12 (m, 1H), 2.85 (t, 2H), 2.69 (t, 2H), 2.47 (buried s, 3H), 245 (s, 3H), 2.21 (m, 2H), 1.88 (s, 1H-OAc peak), 1.29 (d, 6H): MS (EI) for C₂₉H₃₃N₇OSF₂: 566.2 (Mil⁺)

4-[7-(2-Amino[1,3]thiazolo[5,4-b]pyridin-6-yl)-9-methyl-2,3-dibydro-1.4- benzoxazepin-4(5H)-yl]-6,6-dimethyl-5,6,7,8-tetrahydroquinazolin-2-amine, 1H NMR (400 MHz, DMSO-d₆): 8.36 (d, 1H), 7.87 (s, 2H), 7.81 (d, 1H), 7.48 (s, 2H), 5.84 (s, 2H), 4.46 (s, 2), 4.27 (s, 2H), 3.73 (s, 2H), 2.50 (t, 2H), 2.34 (s, 2H), 2.26 (s, 3H), 1.54 (t, 2H), 0.86 (s, 6H); MS (EI) for C₆H₂₉N₇OS: 488 (MH⁺),

6-{4-[2-Amino-5-(trifluoromethyl)pyrimidin4-yl]-9-methyl-2,3,4,5-tetrahydro-1,4- benzoxazepin-7-yl)][1,3]thiazolo[5,4]pyridin-2-amine. ¹H NMR (400 MHz, DMSO-₆): 8.38 (d, 1H), 8.19 (s, 1H), 7.94-7.70 (m, 3H), 7.57 (s, 1H), 7.45 (s, 1H), 6.93 (s. 2H), 4.84 (s. 2H), 4.27 (s. 2H), 3.90 (s. 2H), 2.22 (s. 3H): MS (EI) for C₂₁H₁₈F₃N₇OS: 474 (MH⁺).

6-{4-[4-amino-5-(trifluoromethyl)pyrimidin-2-yl]-9-methyl-2,3,4,5-tetrahydro-1,4- benzoxazepin-7-yl}[1,3]thiazolol[5.4-b]pyridin-2-amine. ¹H NMR (400 MHz. DMSO-d₆): 8.41 (s, 1H), 8.07 (s, 1H), 7.85 (s. 4H), 7.69 (s. 1H), 7.46 (s, 2H); 4.81 (s, 2H), 4.11 (d, 4H), 2.23 (s, 3H); MS (EI) for C₂₁ H₁₈F₃N₇OS: 474 (MH⁺).

6-[9-Methyl-4-(3-methylpyridin-4-yl)-2,3,4,5-tetrabydro-1,4-benzoxazepin-7- yl][1,3]thiazolo[5,4-b]pyridin-2-amine. ¹H NMR (400 MHz, DMSO -d₆): 8.39 (s. 1H), 8.18 (s. 2H), 7.85 (m, 3H), 7.59 (s. 1H), 7.51 (s. 1H), 6.91 (s, 1H), 4.41 (s, 2H) , 4.30-4.22 (m. 2H), 3.65-3.51 (m, 2H), 2.26 (s, 3H), 2.24 (s. 3H): MS (EI) for C_{22H}₂₁N₅OS: 404 (MH⁺),

6-[4-(2-Amino-6-methyl-5-prop-2-en-1-ylpyrimdin-4-yl)-9-methyl-2,3,4,5- tetrahydro-1,4-benzoxazepin-7-yl][3]thiazolo[5,4-b]pyridin-2-amine. ¹H NMR (400 MHz. DMSO-d₆): 8.33 (d, 1H), 7.87 (s, 2H), 7.77 (d. 111), 7.47 (s, 1H), 7.34 (s, 1H), 6.00 (m, 3M), 5.20 (d, 111), 4.98 (d, 1H), 4.43 (s. 2H), 4.25 (s. 2H), 3.68 (s, 2H), 3.16 (s, 2H), 2.25 (s. 3H), 2.10 (s, 3H); MS (EI) for C₂H₂₅N₇OS: 460 (MH⁺),

6-[4-(2-Amino-6-chloro-5-ethylpyrimidin-4-yl)-9-methyl-2,3,4,5-tetrahydro-1,4- benzoxazepin-7-yl][1,3]thiazplo]5.4-b]pyridin-2-amine. ¹H NMR (400 MHz. DMSO-d₆): 8.36 (d, 1 H), 7.86 (s, 2H), 7.81 (d, 1H), 7.48 (s, 2H), 6.46 (s, 2H), 4.57 (s. 2H), 4,31 (s, 2H), 3.77 (s. 2H), 2.53 (m, 2H), 2.25 (s. 3H), 1.14 (t, 3H); MS (EI) for C₂₂H₂₂ClN₇S: 468 (MH⁺).

6-(4-{6-Chloro-2-[{dimethylamino)methyl]-5-ethylpyrimidin-4-yl-9-methyl-2,3,4,5- tetrahydro-1,4-benzoxazeping-7-yl)[1,3]thiazolo[5,4-b]pyridin-2-amine. ¹H NMR (400 MHz, DMSO-d₆): 8.37 (d. 1H), 7.87 (s, 2H), 7.81 (d. 1H),7.54 (d. 1H), 7.46 (d, 1H), 4.74 (s. 2H), 4.42-4.26 (m. 2H), 3.98-3.82 (m. 2H), 3.34 (s, 2H), 2.68 (t. 2H), 2.24 (d. 3H), 2.11 (s. 6H), 1.21 (t. 3H): MS (EI) for C₂₅H₂₀ClN₇OS: 510(MH⁺).

6-{4-[2-{[(1,1 -Dimethylethyl)amino]methyl}-6-methyl-5-(1-melhylethyl)pyrimidin- 4-yl ]-9-methyl-2,3,4,5-tetrahydro-1,4 -benzoxazepin-7-yl}[1,3]thiazdlo[5,4-b]pyridin-2- amine. ¹H NMR (400 MHz, DMSO-d₆): 8.35 (d. 1H), 7.86 (s, 2H), 7.79 (d, 1H), 7.48 (s. 1H), 7.43 (s. 1H), 4.46 (s. 2H), 4.30 (s, 2H), 3.70 (s. 2H), 3.56 (s, 2H), 3.27 (m, 1H), 2.46 (s. 3H), 2.25 (s, 3H), 1.31 (d. 6H), 0.94 (s, 9H); MS (EI) for C₂₉H₃₇N₇OS: 532 (MH⁺)

6-[9-Methyl-4-(2,6,6-trimethyl-5,6,7,8-tetrahydroquinazolin-4-yl)-2,3,4,5-tetrahydro- 1,4-benzoxazepin-7-yl][1,3]thiazolo[5,4-b]pyridin-2-amine. ¹H NMR (400 MHz. methanol- d₄): 8.34 (d, 1H), 7.82 (d. 1H), 7.42.(d. 1H), 7.40 (d. 1H),466 (s, 2H),4.30 (m, 2H), 3,97 (m. 2H), 2,74 (t. 2H), 2.46 (s, 2H), 2.40 (s, 3H), 2,31 (s,3H), 1.66 (t. 2H), 0.91 (s. 6H); MS (EI) for C₂₇H₃₀N₆OS: 487 (MH⁺).

6-[4-(6,6-Dimethyl-5,6-dihydroquinazolin-4-yl)-9-methyl-2,3,4-tetrahydro-1,4- benzoxazepin-7-yl][1,3]thiazolo[5,4-b]pyridin-2-amine. ¹H NMR (400 MHz, methanol- d₄): 8.54 (s. 1H), 8.37 (d, 1H),7.83 (d, 1H), 7.52 (d, 1H), 7,44 (d. 1H), 6,59 (d, 1H), 6.34 (d, 1H), 5.12 (s. 2H), 4.47 (m. 2H), 4.30 (m. 2H), 2.92 (s, 2H), 2.29 (s.3H), 1.08 (s. 6H): MS (EI) for C₂₆H₂₆N₆OS: 471 (MH⁺).

6-[9-Methyl-4-[6-methyl-5(1-methylethyl)-2-(pyrimidin-4- yl]-2,3,4,5-tetrahydro-1,4-benzoxazepin-7-yl][1,3]thiazold[5,4-b]pyridin-2-amine. ¹NMR (400 MHz. methanol-d₆), 8.35 (d. 1H), 7.82 (d. 1H), 7.41 (m, 2H), 4.61 (s, 2H), 4.38 (m, 2H), 4.03 (s. 2H), 3.85 (m. 2H), 3.3S (m, 1H), 301 (m, 4H), 2.56 (s. 3H), 2.31 (s. 3H), 1.91 (s. 3H), 1.88 (m. 4H), 1.39 (d. 6H); MS (EI) for C₂₉H₃₅N₇OS: 530 (MH⁺).

6-(4-{2-[(Dimethylamino)methyl]5-(2,2,2-trifluoroethyl)pyrimidin-4-yl}-9-methyl- 2,3,4,5-tetrahydro-1,4-benzoxazepin7-yl)[1,3]thiazolo[5,4-b]pyridin-2-amine. ¹H NMR (400 MHz, methanol-d₄): 8.44 (d, 1H), 8.27(s, 1H), 7.60 (d, 1H), 7.49 (d, 1H), 7,38 (d, 1H), 4.91 (s, 2H), 4.44 (m. 2H), 4.04 (m, 2H), 3.83 (s, 2H), 3.73 (q, 2H), 2.47 (s, 6H), 2.27 (s, 3H), 1.94 (s. 3H); MS (EI) for C₂₅H₂₆F₃N₇OS: 531 (MH⁺).

6-(4-{2-[(Diethylamino)methyl]-6-methylI-5-(1-methylethyl)pyrimidin-4-yl (-9- methyl-2,3,4,5-tetrahydro-1,4-benzoxazepin-7-yl)[1,3]thiazolo[5.4-b]pyridin-2-amine. ¹H NMR (400 MHz. methanol-d₄): 8.33 (d, 1H), 7.8.1 (d, 1H), 7.39 (m, 2H),4,58 (s, 2H), 4.36 (m. 2H), 3.93 (s, 2H), 3.84 (m. 2-1H), 3.39 (m. 1H), 2.90 (q. 4H), 2,56 (s, 3H), 2.31 (s. 3H), 1.90 (s, 3H), 1.39 (d. 6H), 1.13 (t. 6H); MS (EI) for C₂₉H₃₇N₇S: 532 (MH⁺).

6-{9-Methyl-4-[6-methyl-5-(1-metthylethyl)pyrimidin-4-yl]-2,3,4,5-tetrahydro-1,4- benzoxazepin-7-yl}[1,3]thiazolo[5,4-b]pyridin-2-amine. ¹H NMR (400 MHz, methanol- d₄): 8.66 (d, 1H), 8.45 (s, 1H), 8.01 (d. 1H), 7.56 (d, 1H), 7.46 (d. 1H), 5.10 (s. 2H), 4.52 (m, 2H), 4.16 (m, 2H), 3.21 (m. 1H), 2.62 (s. 3H), 2.27 (s. 3H), 1.43 (d. 6H); MS (EI) for C₂₄H₂₆N₆OS: 447 (MH⁺).

6-(4-{2-[(Dimethylamino)methyl]-5-(1-methylethyl)pyrimidin-4-yl ]-9-methyl- 2,3,4,5-tetrahydro-1,4-benzoxazepin-7-yl)[1,3]thiazolo[5,4-b]pyridin-2-amine. ¹H NMR (400 MHz. DMSO-d₆) δ 8.35 (d, 1H), 8.34 (s, 1H), 7.87 (br. s, 2H), 7.79 (d, 1H), 7.346 (s. 2H), 4.61 (s, 2H), 4.35-4.28 (m, 2H), 3.87-3.81 (m, 2H), 3.38 (s. 2H), 3.14-3.04 (m, 1H), 2.24 (s. 3H), 2.12 (s, 6H), 1.23 (d, 6H): MS (EI) for C₂₆H₃₁N₇OS: 490,(MH⁺),

6(4-{2-[(Dimethylamino)methyl]-5-ethylpyrimidin-4-yl]-9-methyl-2,3,4,5- tetrahydro-1,4-benzoxazepin-7-yl)[1,3]thiazolo[5,4-b]pyridin-2-amine. ¹H NMR (400 MHz, DMSO-d₆) δ 8.36 (d. 1H), 8.12 (s. 1H), 7.86 (s. 2H), 7.80 (d, 1H), 7.56 (d, 1H), 7.44 (d. 1H), 4.72 (s. 2H), 4.33-4.24 (m, 2H), 3.99-3.92 (m, 2H), 3.36 (s. 2H), 2.67 (q. 2H), 2.23 (s, 3H), 2.12 (s. 611), 1.14 (t. 3H); MS (EI) for C₂₅H29N₇OS: 476 (MH⁺).

6-(4-{2-[(Dimethylamino))methyl]-5,6-diethylpyrimidin-4-yl]-9-metbyl-2,3,4,5- tetrahydro-1,4-benzoxazepin-7-yl)[1,3]thiazolo-[5,4-b]pyridin-2-amine. ¹NMR (400 MHz. DMSO-d₆) δ 8.35 (d, 1H), 7.86 (s. 2H), 7.79 (d, 1H), 7.49 (s, 1 H), 7.46 (s, 1H),4.57 (s, 2H), 4.34-4.27 (m. 2H), 3.83-3.76 (m, 2H), 3.36 (s. 2H), 2.70-2.58 (m. 4H), 2.24 (s. 3H), 2.15 (s, 6H), 1.19-1.09 (m, 6H); MS (EI) for C₂₇H₃₃N₇S: 504 (MH⁺),

6-{9-Methyl-4-[5-(1-methylethyl)-2-(pyrrolidin)-1-ylmethyl)pyrimidin-4-yl]-2,3,4,5- tetrahyclro-l,4-benzoxazepin-7-yl}[3]thiazolo[5.4-b]pyridin-2-amine. ¹H NMR (400 MHz. DMSO-cW S 8.34 (d. 1H), 8.33 (s. 1H), 7.86 (s. 2H), 7.78 (d. 1H), 7.48-7.43 (111. 2H), 4.62 (s, 2H), 4.36-4.29 (m. 2H), 3.86-3.80 (m, 2H), 3.52 (s. 2H), 3.15-3.05 (m, 1H), 2.44-2.38 (m, 4H), 2.24 (s. 3H), 1.61-1.53 (m. 4H), 1.23 (d,6H); MS (EI) for C₂₈H₃₃N₇OS: 516 (MH⁺).

6-(9-Methyl-4- [6-methyl-5-(1 -methylethyl)-2-[(4-methylpiperazin-1- yl)methyl]pyrimidin-4-yl}-2,3,4,5-tetrahydro-1,4-benzoxazdepin-7-yl)[1,3]thiazolo[5,4- b]pyridin-2-amine. ¹NMR (400 MHz. DMSO-d₆) δ 8.33 (d, 1H), 7.88 (s, 2H), 7.77 (d, 1H), 7.47 (d, 1H), 7.38 (d, 1 H), 4.43 (s, 2H), 4.38-4.24 (m, 2H), 3.76-3.57 (m, 2H), 3.36 (s, 2H), 3.31-3.23 (m, 1H), 2.46 (s, 3H), 2.41-2.14 (m. 1H), 2.10 (s. 3H), 1.31 (d. 6H): MS (EI) for C₃₀H₃₈N₈OS: 559 (MH⁺).

6-(4-{2r-[(Dimethylamino)methyl]-5-ethyl-6-methylpyrimidin-4-yl}-9-methyl-2,3,4,5- tetrahydro-1,4-benzoxazepin-7-yl)[1,3]thiazolo[5,4-b]pyridin-2-amine. ¹H NMR (400 MHz. DMSO-d₆) δ 8.33 (m. 1H), 7.85 (s. 2H), 7.77 (m, 1H), 7.44 (t. 2H), 4.54 (d. 2H), 4.27 (d. 2H), 3.76 (d, 2H), 2.59 (q, 2H), 2.33 (s. 3H), 2.22 (d. 3H), 2.11 (s, 6H), 1,88 (s, 2H), 1.11. (ni, 3H): MS .(EI) for C₂₆H₃₁N₇OS: 490.2 (MH⁺).

6-(4{2-[(Dimethylamino)methyl]-6-isopropyl-5-methylpyrimidin-4-yl}-9-methyl- 2,3,4,5-tetrahydro-1,4benzoxazepin-7-yl)[1,3]thiazolo[5,4]pyridin-2-amine. ¹NMR (400 MHz, DMSO-d6) 8 8.33(1. 111),,7.86 (s, 211), 7.78 (t. 111), 7.51 (s. Ill), 7.44 (d. 1H); 4.57(s, . 2H), 4.30 (s, 2H), 3.81 (s, 2H), 3.62 (s; 2H); 3.18 (m, 1 H), 2.32 ( s, 6H), 2.21 (d, 6H), 1.14 (t, 6H): MS (EI) for C₂₇H₃₃N₇N₇OS: 490.2 (MH⁺).

6-(4-{2-[(Dimethylamino)methyl]-6-methyl-5-(1-methylethyl)pyrimidin-4-yl}-9- methyl-2,3,4,5-tetrahydro-1,4-benzoxazepin-7-yl)[1,3]thiazolo[5,4-b]pyridin-2-amine. ¹H NMR (400 MHz. DMSO-d₆): 8,35 (d, 1H); 7.88 (br. s, 2H), 7.78 (d, 1H), 7.48 (d. 1H), 7.40 (d. 1H), 4.40(s, 2H), 4.28 (m. 2H), 3.67 (m. 2H),3.35 (s. 2H), 3.27 (m, 1H), 2.45 (s, 3H), 2.25:(s, 3H), 2.14 (s, 6H), 1.30 (d,6H); MS (EI) for C₂₇H₃₃N₇OS: 504 (MH⁺).

6-(9-Methyl-4-{6-methyl-5-(1-methylethyl)-2-[(methoxyloxy)methyl]pyridin-4-}- 2,3,4,5-tetrahydro-1,4)benzoxazepin-7-yl)[1.3]thiazolo[5,4-b]pyridin-2-amine. ¹ H NMR (400 MHz, DMSO-d₆): 8.35 (d, 1H), 7.87 ( s, 2H), 7.79 (d. 1H), 7.49 (d, 114),7.42 (d, 1H), 4.41 (s, 2H), 4.30 (s. 2H), 4.27 (m. 2H), 3.68 (m, 2H), 3.32 (m, 1H), 3.26 (s. 3H), 2.47 (s. 3H), 2.26 (s, 3H), 1.29 (d,6H); MS (EI) for C₂₆H₃₀N₆O₂S: 491 (MH⁺).

6-(9-Methyl-4-[(7S)-7-methyl-5,6,7,8-tetrahydroquinazolin-4-yl]-2,3,4,5-tetrahydro- 1,4-benzoxazepin-7-yl}[1,3]thiazolo[5,4-b]pyridin-2-amine. ¹M NMR (400. MHz. CD₃OD) δ 8.58 (d. 1H), 8.52 (s. 1H), 7.96 (d, 1H), 7.57 (d, 1H), 7.46 (d. 1H), 5.20 (d. 1H), 5,13 (d. 1H), 4.52 (m. 2H), 4.29 (m, 2H), 2.98 (m, 1H), 2,93 (m. 1H), 2.74 (m. 1H), 2.38 (m. 1H), 2.29 (s, 3H), 2.00 (m, 2H), 1.27 (m, 1H), 1.12.(d, 3H); MS (ES) C₂₅H₂₆N₂₆N₆OS: 459 (MH⁺),

2-Amino-6-[7-(2-amino[1,3]thiazold[5,4-b]pyridin-6-yl)-9-methyl-2,3-dihydro-1,4- benzoxazepin-4(5H)-yl]pyridine-3,5-dicarbonitrile. ¹H NMR (400 MHz. d₆-DMSO) δ 8.38 (d, 1H), 8.12 (d. 1H), 7.85 (s. 2H), 7.84 (d, 1H), 7.68 (d, 1H), 7.48 (d. 1H); 7.48 (bs. 2H), 5.00 (s, 2H), 4.27 (m. 4H), 2.24 (s, 3H); MS (ES) C₂₃H₁₈N₈OS: 455 (MH⁺).

2-Amino-6-[7-(2-amino[1,3]thiazolo[5,4-b]pyridin-6-yl)-9-methyl-2,3-dihydro-1,4- benzoxazepin-4(5H)-yl]pyridine-3,5-dicarbonitrile. ¹H NMR (400 MHz, d₆-DMSO) δ9.34 (d, 1H), 8.43 (d. 1H), 8.16 (bs, 2H), 7.87(d. 1H), 7.66 (d. 1H), 7.45 (d. 1H), 7.42 (bs, 2H), 5.00 (s. 2H), 4.28 (m, 4H), 2.37 (s, 3H), 2.23 (s. 3H): MS (ES) C₂₄H₂₀N₈OS: 469 (MH⁺).

Proceeding according to the method of example 1 and replacing tert-butyl 7- bronio-9-methyl-2,3-dihydro-1.4-benzoxazepine-4(5H)-carboxylate with tert-butyl 7-bromo- 9-ethyl-2,3-dihydro-1,4-benzoxazepine-4)5h)-carboxylate, the following compounds of the invention were prepared:

6-{4-[2-Amino-6-methyl-5-(1-methylethyl)pyridin-4]-9-ethyl-2,3,4,5- tetrahydro-1,4-benzoxazepin-7-yl}[1,3]thiazolo-8 5,4-b]pyridin-2-mine. ¹H NMR (400MHz. d₆-DMSO) δ 8.35 (d, 1H), 7.87 (s, 2H), 7.80 (d, 1H), 7.49 (d, 1H), 7.39 (d, 1H), 6.06 (s, 2H), 4.23 (m 2H), 4.23 (s. 2H), 3.54 (m. 2H), 3.22 (m, 1H), 2.68 (q, 2H), 230 (s. 3H), 1.26 (d, 6H), 1.20 (t. 3H); MS (ES) for C₂₅H₂₉N₇OS: 476 (MH⁺).

Proceeding according to the method of example 1 and replacement of starting reagents in step 2 with tert-butyl 7-bromo-9-chloro-2,3-dihydro-1,4-benzoxazepine-4(5H)- carboxylate and N,[6-(4,4,5,5-tetramethyl-1,3,2-dioxaborolan-2-yl)[1,3]thiazolo[5,4- b]pyriidin-2-yl]acetamide, the following compounds of the invention were prepared:

6-{4-[2-amino-6-methyl-5-(1-methylethyl)pyrimidin-4-yl]-9-chloro-2,3,4,5- tetrahydro-1,4- benzoxazepin-7-yl}[1,3]thiazolo[5,4-b]pyridin-2-amine (EXEL-04519289). ¹H NMR (400 MHz, CD₃OD) δ 8.63 (d, 1H),7.99 (d. 1H), 7.70 (d, 1H), 7.66 (s. 1H), 4.94 (s. 2H), 4.51 (m, 2H), 4.06 (m, 2H ), 3.06 (m, 1H), 2.45 (s. 3H), 1.37 (d. 6H); MS (ES) for C₂₃H₂₄ClN₇OS: 482 (MH⁺).

6-{4-[2-{[(1,-dimethylethyl)(methyl)amino]methyl}-6-methyl-5-(1- methylethyl)pyrimidin-4-yl]-9-methyl-2,3,4,5-tetrahydro-1,4-benzoxazepin-7- yl){[1,3]thiazo[5,4-b]pyridin-2-amine. ¹H NMR (400 MHz, d₆-DMSO) δ 8.34 (d, 1H),7.86 (s, 2H), 7.77 (d. 1H),7,47(s, 1H),7,39 (s. 1H), 4.40(s, 2H), 4.30 (s. 2H), 3,68 (s, 2H), 3.39 (m, 3H), 2.46 (s. 3H), 2.25 (s. 3H), 2.13 (s,3H), 1.31 (d, 6H), 0.98 (s,9H); MS (EI) for C₃₀H₃₉N₇OS: 546 (MH⁺),

6-[4-[2-{[(2.2-difluoroethyl)amino]methyl}-6-methyl-5-(1-methylethyl)pyrimidin-4- yl]-9-methy1,2,3,4,5-tetrahydro-1,4-benzoxazepin-7-yl}[1,3]thiazolco[5.4-b]pyridin-2-amine.

¹H NMR (400 MHz. methanol-d₆): 8.70 (d. 1H), 8.07 (d, 1H), 7.62 (d, 1H), 7.55 (d, 1H), 6,29 (m, 1H), 5.10 (s, 2H), 4.60 (m, 2H), 4.53 (s. 2H), 4.20 (m. 2H), 3.65 (m. 2H), 3.16 (m,1H), 2.65 (s, 3H), 2.31 (s. 3H), 1.41 (d. 6H), MS (EI) for C₂₇H₃₁F₂N₇OS: 54.0 (MH⁺).

6-{9-methyl-4-[6-methyl-5-(1-methylethyl)-2-{[(2,2,2- trifluoroethyl)amino]methyl}pyrimidin-4-yl]-2,3,4,5-tetrahydro-1,4-benzoxazepin-7- yl][1,3]thiazolo[5,4-b]pyimidin-2-amine. ¹H NMR (400 MHz. d₆-DMSO): 8.35 (d. 1H), 7.86(s. 2H), 7.79 (d. 1H), 7.49 (s, 1H), 7.44 (s. 1H), 4.46 (s, 2H), 4.31 (s. 2H), 3.70 (s, 4H), 3.17 (d. 3H), 2.70 (d, 2H), 2.47 (s, 3H), 2.25 (s, 3H), 1,89 (s, 1H), 1.34 (s, 1H), 1.31 (d. 6H), MS (EI) for C₂₇H₃₀F₃N₇OS: 558.2 (MH⁺).

6-{4-[2,6-dimethyl-5-(1-methyl)pyridin-4-yl]-9-methyl-2,3,4,5-tetrahydro- 1,4-benzoxazepin-7-yl}[1,3]thiazolo[5,4-b]pyridin-2-amine. ¹H NMR (400 MHz, d₆- DMSO): 8.35 (d. 1H), 7.88 (s, 2H), 7.79 (d. 1H), 7.51 (d, 1H), 7.42 (d, 1H), 4.35 (s. 2H), 4.31-4.18 (m, 2H), 3.69-3.59 (m, 2H), 3.32-3.22 (m, 1H); 2.44 (s, 3H), 2.35 (s, 3H), 2.27 (s. 3H), 1.29 (d, 6H), MS (EI) for C₂₅H₂₈N6OS: 461 (MH⁺).

{4-[7-(2-amino[1,3]thiazolo[5,4-b]pyridin-6-yl)-9-methyl-2,3-dihydro-1,4- benzoxazepin-4(5H)-yl]-6-methyl-5-(1-methylethyl)pyrimidin MS (ES) for C₂₆ H₂₇N₇OS: 486 (MH⁺) ¹H NMR (400 MHz. d₆-DMSO) δ 8.36 (s. 1 H), 7.83 (s. 2H), 7.80 (s, 1H), 7.51 (s. 1H), 7.42 (s, 1H), 4.46 (s, 2H), 4.32 (s, 1H), 4.09 (s, 2H), 3.74 (m, 2H), 3.23(m, 1H), 2.48 (s, 3H)>2.27 (s. 3H), 1.31 (d, 6H). N-(5-{4-[2-amino-6-methyl-5-(1-methylethyl)pyrimidin-4-yl]-9-methyl-2,3,5,5-tetrahydro- 1,4-benzoxazepin-7-yl}-1,3-thiazol-2yl-2-yl)acetamide. ¹H NMR 400 MHz, d₆-DMSO): 7.69 (s, 1H), 7.38 (s, 1H), 7.23 (s, 1H),5.97 (s, 2H), 4.19 (br ir, 2H), 4.16 (s, 2H), 3.49 (br r, 2H), 3.15 (m, 1H), 2.27 (s. 3H), 2.21 (s, 3H), 2.12 (s. 3H), 1.22 (d. 6H), MS (EI) for C₂₃H₂₈N₆O₂S: 454 (MH⁺)

6- ]9-methyl-4-[2-methyl-5-(1 -methylethyl)pyrimidin-4-yl ]-2,3,4,5-tetrahydro-1.4- benzoxazepin-7-yl}[1,1,3]thiazolo[5,4-b]pyridin-2-amine. ¹ H NMR (400 MHz, d₆-DMSO): 8.35 (d, 1H), 8.28 (s. 1H), 7.89 (s, 2H), 7.79 (d, 1H), 7.51-7.47 (m. 1H), 7.47-7.43 (m, 1H), 4.56 (s, 2H), 4.37-4.24 (m, 2H), 3.87-3.75 (m, 2H), 3.13-2.98(m, 1H), 2.38 (s, 3H), 2.25 (s, 3H), 1.21 (d, 6H), MS (EI) for C₂₄H₂₆N₆OS: 447(MH⁺).

6-[4-[6-chloro-5-(1-methylethyl)pyrimidin-4-yl]-9-methyl-2,3,4,5-tetrahydro-1,4- benzoxazepin-7-yl}[1,3]thiazolo[5.4-b]pyridin-2-amine. ¹H NMR (400 MHz, d-fi DMSO): 8.33 (m. 2H), 7.86 (s, 2H), 7.78 (d. 1H), 7.50 (s, 1H), 7145 (s, 1H), 4.56 (s. 2H), 4.34 (br s. 2H), 3.78 (br s, 2H), 3.19 (m, 1H), 2.25 (s. 3H), 1.37 (d, 6H), MS (EI) for C₂₃H₂₃ClN₆OS: 467.1 (MH⁺), 6-[4-(5-ethenyl-6-methylpyrimidin-4-yl)-9-methyl-2,3,4,5-tetrahydro-1,4- benzoxazepin-7-yl][1.3]thiazolo[5.4-b]pyridin-2-amine. ¹H NMR (400 MHz, d₆-DMSO): 8.32 (dd, 2H), 7.86 (s. 2H), 7.78 (t. 1H), 7.44 (d, 1H), 7.38 (d, 1H), 6.75 (dd, 1H), 5.58 (dd, 1H), 5.37 (dd, 1H), 4.82 (s. 2H), 4.29 (s. 2H), 3.90 (s; 2H), 2.33 (s. 3H), 2.22 (s, 3H), MS (EI) for C₂₃H₂₂N₆OS: 431.1 (MH⁺),

6-{9-methyl-4-[5-(1 -methylethyl)pyrimidin-4-yl]-2,3,4,5-tetrahydro1,4- benzoxazepin-7-yl}[1,3]thiazolo[5,4-b]pyridin-2-amine. ¹H NMR (400 MHz. d₆-DMSO): 8.48(d. 1H), 8.41 (d. 1H), 8.35 (t. 1H), 7.87 (s. 2H), 7.79 (t, 1H),7.49(d, 1H), 7.42 (d, 1H), 4.61 (s, 2H), 4.35 (s. 2H), 3.82 (s, 2H), 3.9 (dd, 1H), 2.25 (s. 3H), 1.22 (t, 6H,),. MS (EI) for C₂₃H₂₄N₆OS: 433.2 (MH⁺),

6-{4-[2-amino-6-methyl-5-(1-methylethyl)pyrimidin-4-yl -2,3,4,5-tetrahydro-1.4- benzoxazepin-7-yl}[1,3[thiazolo[5.4-b]pyridin-2-amine--d_{4.} ¹H NMR (400 MHz, DMSO-d6): δ 8.32 (t 1J), 7.85 (s. 2H), 7.77 (t. 1H), 7.55 (d, 2H), 7.05 (t. 1H), 5.98 (s. 2H), 4.26 (s, 2H), 3.15 (m. 1H), 2.27 (s, 3H), 1.87 (s. 3H), 1.22 (d, 6H), MS (EI) for C₂₃H₂₁N₇OSD: 452.2 (MH⁺).

6-{4]-[2-amino-6-methyl-5-(1-methylethyl)pyrimidin-4-yl]-2,3,4,5-tetrahydro-1,4- benzoxazepin-7-yl{[1,3]thiazolo[5,4-b]pyridin-2-amine-d_{6.} ¹H NMR (400 MHz, DMSO-d₆) δ 8.25:(s, 1H), 7,84 (s, 2H), 7.79 (s. 1H), 7.05 (d. 1H), 6.19 (bs. 1H), 4.01 (s, 1H), 3.12 (m. 1H), 2.28 (s, 3H), 1.21 (s, 6H), MS (EI) for C₂₃H₁₉N_{7OSD}_{6:} 454.2 (MH⁺).

6-{9-methyl-4-[6-methyl-5-(1-methylethenyl)pyrimidin-4-yl]-2,3,4,5-tetrahydro-1,4- benzoxazepin-7-yl}[1.3]thiazolo[5.4-b]pyridin-2-amine. ¹H NMR (400 MHz. d₆-DMSO): 8.32 (d. 2H), 7.86 (s. 2H), 7.77 (t. 1H), 7.44 (s, 1H), 7.39 (d. 1H), 5.48 (s, 1H), 5.13 (s, 1H), 4.84 (s, 2H), 4.23 (d. 2H), 4.05 (s. 2H), 2.28 (d. 3H), 2.22 (s. 3H), E92 (s. 3H), 1.88 (s. 2H), MS (EI) for C₂₄H₂₄N₆OS: 445.2 (MH⁺).

1-{4-[7-(2-amino[1,3]thiazolo[5,4-b]pyridin-6-yl)-9-methyl-2,3-dihydro-1,4- benzoxazepin-4(5H)-yl]-6-methylpyrimidin-5-yl}ethanone. ¹H NMR (400 MHz, d₆-DMSO): 840:(s, 1H), 8.35 (d, 1H), 7.8 (s, 2H), 7.80 (d. 1H), 7.45;(d. 1H), 7.37 (d. 1H), 4.80 (s. 2H), 4.38-4.25 (m. 2H), 3,90-3.69 (m. 2H), 2.43 (s. 3H), 2.25 (s. 3H), 2.20 (s, 3H), MS (EI) for C₂₃H₂₂N₆O₂S: 447.1 (MH⁺).

6-[4-[[2- (2-fluoroethyl)amine]methyl}-6-methyl-5-(1-methylethyl)pyrimidin-4-yl]- 9-methyl-2,3,4,5-tetrahydro-1,4-benzoxazepin-7-yl][1,3]thiazol]5.4-b]pyridin-2-amine. ¹H NMR (400 MHz, CD₃OD) δ 8.71 (d, 1H); 8.11 (d, 1H), 7.69 (s. 1H); 7.55 (s. 1H), 5.16 (s. 2H), 4.75 (m,2H); 4.62 (t, 2H), 4.52 (s, 2H), 4.23 (t, 2H), 3.50 (dt, 2H), 3.16 (m, 1H), 2.66 (s. 3H), 2.30 (s, 3H), 1.43 (d, 61-1), MS (ES) for C₂₇H₃₂₁N₇OS: 522(MH⁺).

6-(9-methyl-4-{6-methyl-5-[2-(methyloxy)ethyl]-2-(pyrrolidin-1-ylmethyl)pyrimidin- 4-yl}-2,3,4,5-tetrahydro-1.4-benzoxazepin-7-yI)[1,3]thiazolo[5.4-b]pyridin-2-amine. ¹H NMR (400 MHz. d₆-DMSO): 8.37 (d. 1H), 7.87 (s. 2H), 7.83 (d, 1H), 7.49 (d. 2H), 4.56 (s, 2H), 4.35-4.24 (m, 2H), 3.83-3.72 (m. 2H), 3.54 (t. 2H), 3.50 (s, 2H), 3.21 (s. 3H), 2.91 (t, 2H), 2.48-2.41 (m. 4H), 2.38 (s. 3H), 2.25 (s. 3H), 1.64-1.55 (m, 4H), MS (EI) for C₂₉H₃₅N₇O₂S: 554 (MH⁺).

6-{9-methyl-4-[6-methyl-5-(1-methylethyl)-2-(trifluoromethyl)pyrimidin-4yl]- 2,3,4,5-tetrahydro-1,4-benzoxazepin-7-yl}[1,3]thiazolo[5.4-b]pyridin-2-amine. ¹H NMR (400 MHz. d₆-DMSO) δ 8.35 (d. 1H), 7.88 (s. 2H), 7.79 (d, 1H), 7.48 (d, 2H), 4.58 (s, 2H), 4.32 (s, 2H), 3.81 (s, 2H), 3.24 (dd, 1H), 2.55 (s, 3H), 2.24 (d. 3H), 1.34 (d, 6H), MS (EI) for C₂₅H₂₅F₃N₆OS: 515 (MH⁺).

6-(9-methyl-4-{6-metyl-5-[2-(methyloxy)ethyl]pyrimidin-4-yl]-2,3,4,5-tetrahydro- 1,4-benzoxazepin-7-yl)[1,3]thiazolo[5.4- b]pyridin-2-amine. ¹H NMR (40.0 MHz, methanol- d₄): 8.33 (s, 1H), 8,36 (d, 1H), 7.84 (d. 1H), 7.45 (d. 1H), 7.41 (d. 1H), 4.66 (s, 2H), 4.62 (br s. 2H), 4.35(m, 2H), 3.89 (m. 2H), 3.58.(t, 2H), 3.24 (s.3H), 3.04 (t, 2H), 2.47 (s, 3H), 2.31 (s, 3H), MS (EI) for C₂₄H₂₄N₆O₂S: 463 (MH⁺).

6-{4-[2-amino-6-methyl-5(1-methylethenyl)pyrimidin-4-yl]-9-methtyl-2,3,4,5- tetrahydro-1,4-benzoxazepin-7-yl][1,3]thiazolo[t,4-b]pyridin-2-amine. ¹H NMR (400 MHz. d₆-DMSO): 8.34 (d. 1H), 7.85 (s. 2H), 7.78 (t, 1H), 7.53-7.36 (m. 2H), 5.98 (s, 2H), 5.36 (s. 1H), 4.99 (s, 1H), 4.72 (s. 2H), 4,15 (s, 2H), 3.97 (s, 2H), 2.23 (s. 3H), 2.09 (s, 3H), 1.89 (s. 1H), 1.87 (s,3H), MS (EI) for C₂₄H₂₅N₇OS: 460.2 (MH⁺),

2-{4-[7-)2-amino[1,3]thiazolo[5,4--b]pyridine-6-yl)-9-methyl-2,3-dihydro-1,4- benzoxazepin-4(5H)-yl]-6;-chloropyrimidin-5-yl)propan-2ol. ¹H NMR (400 MHz, methanol-d₄): 8.33 (d. 1H), 8.09 (s. 1H), 7.80 (d. 1H), 7.43 (d. 1 H), 7.36 (d. 1H), 4.80 (s, 2H), 4.62 (s. 3H), 4.22 (m, 2H), 3.92 (m, 2H), 2.26 (s. 3H), 1.80 (s. 6H), MS (EI) for C₂₃H₂₃ClN₆O₂S: 483 (MH⁺).

6-(4-{2,6-dimethyl-5-[2-(methyloxy)ethyl]pyrimidin-4-yl{-9-metyl-2,3,4,5- tetrahydro-1,4-benzoxazepin-7-yl)[1,3]thiazolo[5,4-b]pyridin-2-amine. ¹H NMR (400 MHz, d₆-DMSO): 8.38 (d, 1H), 7.87 (s, 2H) 7.84 (d. 1H); 7.55-7.47 (m,2H),4.51 (s,2M), 4.30- 4.22 (m, 2H), 3.80-3.73 (m, 2H) 3.51(t. 2H); 3.20 (s, 3H), 2.89 (t. 2), 2.36 (s. 6H), 2.26 (s. 3H), MS (EI) for C₂₅H₂₈N₆O₂S: 477 (MH⁺)

6-{4-[2-azetidin-3-yl-6-methyl:-5-(1-methyl)pyrimidin-4-yl}-9-methyl-2,3,4,5- tetrahydro-1,4-benzoxazepin-7-yl}[1,3]thiazolo[5,4-b]pyridin-2-amine. ¹H NMR (400 MHz, d₆-DMSO): 8.36 (d. 2H), 7.88 (s, 2H), 7.80(d. 1 H), 7.50 (s. 1H), 7.45 (s, 1H), 4.46 (s. 2H), 4.32 (m, 2H), 3.80 (s, 2H), 3.72 (m, 2H), 3.60 (m 2H), 3.26 (m. 1H), 2.46 (s, 3H), 2.25 (s. 3H), 1.31 (d. H), MS (EI) for C₂₃H₃₁N₇OS: 502 (MH⁺),

6-{4-[2-(aminoethyl)-6-methyl-5-(1-methylethtyl)pyrimidin-4-yl]-9-methyl-2,3,4,5- tetrahydro-1,4-benzoxazepin-7-yl}[1,3]thiazolo[5,4-b]pyridin-2-amine. ^{‘}H NMR (400 MHz, d₆-DMSO): 8.35 (d, 1H), 7.87 (br s, 2M), 7.80 (d, 1H), 7,50 (br d, 1H), 7.45 (br d, 1H), 4.45(s, 2H), 4.29 (br t. 2H) 3.72 (br s, 2H), 3.60 (s, 2H), 3.28 (m, 1H), 2.47 (s. 3H), 2.26 (s, 3H), 1.86 (s, 6H-OAc peak), 1.31 (d, 6H), MS (EI) for C₂₅H₂₉N₇OS: 476.2 (MH⁺), 6-(9-methyl-4-[2-methyl-5-[2-(methyloxy)ethyl]pyrimidin-4-yl]-2,3,4,5-tetrahydro- 1,4-benzoxazepin-7-yl)[1,3]thiazolo[5,4-b-]pyridin-2-amine. ¹H NMR (400 MHz, d₆- DMSO): 8.37 (d. 1H), 8.11 (s. 1H), 7.87 (s. 2H), 7.82 (d. 1H), 7.53 (d. 1H), 7.48 (d, 1H), 4.66 (s, 2H), 4.35-4.23 (m, 2), 3.96-3.85 (m. 2H), 3.54 (t, 2H), 3.21 (s, 3H), 2.87 (t, 2H), 2.37 (s. 3H), 2.24 (s. 3H), MS (EI) for C₂₄H₂₆N₆O₂S: 463 (MH⁺).

6-(9-methyl-4-{6-methyl-2-[(methylamino)methyl]-5-(1-methylethyl)pyrimidine-4- yl}-2,3,4,5-tetrahydro-1,4-benzpxazepin-7-yl)[1,3]thiazolo[5,4-b]pyridin-2-amine. ⁺H NMR (400 MHz, d₆-DMSO) δ 8.3.6 (d, 1H), 7,88 (s. 2H), 7.80 (d. 1H), 7.58 - 7,43 (in, 2H), 4.48 (s, 2H), 4.31 (d. 2H), 3.72 (s.2H), 3.56 (s, 2H), 3.26 (m. 1H), 2,45 (s,3H), 2.24 (s. 3H), 2.18 (s. 3H), 1.31 (d, 6H), MS (EI) for C₂₆H₃₁N₇OS: 490(MH⁺).

6-[4-(2,6dimethyl-5-prop-2-yn-1-ylpyrimidin-4-yl)-9-methyl-2,3,4,5-tetrahydro-1,4- benzoxazepin-7-yl]]1,3]thiazolo[5,4-b]pyridin-2-amine. ¹NMR (400 MHz. d₆-DMSO): 8.36 (d, 1H), 7.88 (s. 2H), 7.80 (d. 1H), 7.67 (d. 1H), 7.53 (d. 1H), 4.62 (s, 2H), 4.35-4.29(m. 2H), 3.88-3.82 (m, 2H), 3.39-3.35 (m. 2H), 3.28-3.24(m. 1H), 2.41 (s, 3H), 2.37 (s. 3H), 2.27 (s. 3H), MS (EI) for C₂₅H₂₄N₆OS: 457 (MH⁺).

6-[4-(5-but-2-yn-1-yl-2,6-dimethylpyrimidin-4-yl)-9-methyl -2,3,4,5-tetrahydro-1,4- benzoxazepin-7-yl][1.3]thiazolo[5.4-b]pyridin-2-amine. ¹H NMR (400 MHz. DMSO-d₆) ε 8.34 (t, 1H), 7.88 (s4H), 7.77 (t, 1H), 7,64 (t. 1 H), 7.50 (d, 1H), 4.60 (s, 2;H), 4.30(s, 2H), 3.81 (s, 2H), 3.28 (s, 2H), 2.38, 3H), 2.34(s, 3H), 2,24 (s. 3H), 1.85 (s, 3H), MS (EI) for C₂₆H₂₆N₇OS: 471.2 (MH⁺).

6-(4-[2,6-dimethyl-5-[1 -(methyloxy)ethyl]pyrimidin-4-yl}-9-methyl-2,3,4,5- tetrahydro-1,4-benzoxazepin-7-yl)[1,3]thazolo[5,4-b]pyridin-2-amine. ¹H NMR (400 MHz, d₆-DMSO): 8.36 (d. 1H), 7.88 (s, 2H), 7.80(d. 1H),7.54 -7.41 (m. 2H), 4.66-4.50 (m. 2H), 4.39 (dd, 2H), 4.24 - 4.07 (in. 1H), 3.88 - 3.77 (m. 1H), 3.70 (dd. 1H), 2.94 (s. 3H), 2.45 (s. 3H), 2.35 (d. 3H), 2.30 - 2,18 (m, 3H), 1.58 (d, 3H), MS (EI) for C₂₅H₂₈N₆O₂S: 477.2 (MH⁺).

6-(4-{2,6-dimethyl-5-[(methyloxy)methyl]pyrimidin-4-yl }-9-methyl-2,3,4,5- tetrahydro-1,4-benzoxazepin-7-yl)[1.3 ]thiazolo[5.4-b]pyridin-2-amine. ¹H NMR (400 MHz, df,-DMSO): 8.36 (d, 1H), 7.87 (s, 2H), 7.79 (d, 1H), 7.47 (s. 2H), 4.75 (s. 2H), 4.37 - 4.26(m. 2H), 4.20 (s. 2H), 3.94 (d, 2H), 3.34 (s. 31-1), 2.32 (d, 6H), 2.24 (s, 3H), MS (EI) for C₂₄H₂₆N₆O₂S: 463.2 (MH⁺),

6- (4-[2-(difluoromethyl)-6-methyl-5-(1-methylethyl)pyrimidin-4-yl]-9-methyl 2,3,4,5-tetrahydro-1,4-benzoxazepin-7-yl}[1,3]thiazolo[5.4-b]pyridin-2-amine. ¹H NMR (400 MHz. dr,-DMSO) 5 8.36 (d, 1H), 7.86 (s, 2H), 7.80 (d. 1H), 7.50 (d, 1H), 7.45 (d. 1H), 6.64. (t. 1H), 4.49 (s. 2H), 4.29 (i, 2H), 3.75 (t. 2H); 3.25 (m, 1H), 2.54 (s. 3H), 2.26 ;(s. 3H), 1.33 (d. 6H), MS (ES) for C₂₅H₂₆F₂N₆OS: 497 (MH⁺).

6-[4-(2-amino-5-ethynyl-6-methylpyrimidin-4-yl)-9-methyl-2,3,4,5-tetrahydr-1,4- benzoxazepin-7-yl][3]thiazolo[5,4-b]pyridin-2-amine. ¹H NMR (400 MMz. d6-DMSO) δ 8.34 (d, 1H). 7.86 (s, 2H), 7.78 (d, 1H),7.58 (s, 1H); 7.44(s. 1H); 6.49 (s, 2H), 5.07 (s, 2H), 4,67(s, 1H), 4.32 (s, 2H), 4.6 (s, 2H), 2.23 (s,6H), MS (EI) for C₂₃H₂₄N₇OS: 444(MH⁺).

6-[9-methyl-4-[6-methyl-5-(1-methylethyl)-2-pyrrolidine-2-ylpyrimidin-4-yl]-2,3,4,5- terahydro-1,4-benzoxazepin-7-yl}[1,3]thiazolo[5.4-b]pyridin-2-amine. ¹H NMR (400 MHz. d,,-DMSO): 8.37 (d. 1H), 7.87 (s, 2H), 7.81 (d. 111), 7.47 (s. 2H),. 4.54 (s, 2H), 4.32 (in. 2H), 3.91 (in. 1H), 3.73 (m. 211), 3.23 (m, 111), 2.84-(m. 1H), 2.63 (m. 1H), 2.45 (s, 3H), 2.22 (s, 3H), 1.98(ni. 1H), I.52(m. 3H), 1.30 (dd. 6H), MS (EI) IMC2sH3% OS: 516 (MH⁺),

6-(4-{2-[(2S)-4,4-diflupropynglidin-2-yl]-6-methyl-5-(1-methylethyl)pyrimidin-4- yl}-9-methyl-2,3,4,5-tetrahydro-1,4-benzoxazepin-7-yl)[1,3]thiazolo[5,4-b]pyridine-2-amine. ¹H NMR (400MHz. dj-McOH): 8.36(d, 1H), 7.83:(d. 1H), 7.41 (s. III), 7.40 (s. 1H), 4.54( s, 2H), 4.36 (m, 2H), 4.21 (ir. 1H), 3.84 (tr.2H), 3.35 (in. 1H), 3H)(q. 1H), 2.96 (q, 1H), 2.58-2.47 (in.; 1H),2.52(s, 3H), 2.27 (s, 3H); 2.23-2.11 (m, 1H), 1.35 (dd. 6H); MS (EI) for C₂₈ H₃₁F₂N₇OS:. 553 (MH⁺).

6-{9-methyl-4[6-(methylamino)-5-nitripyridimin-4-yl-]-2,3,4,5-tetrahydro-1,4- benzoxazepin-7-yl][1,3]thiazolo[5,4-b]pyridine-2amine. ¹H NMR (400 MHz, d₆-DMSO):

9.98 (s, 1H); 7.36 (s, 1H),7.29(d,2H), 7.10 (d, 2H), 6.92 (s, 1H), 6.80 (d, 1H), 4.40 (s, 2H); 4.22(m 1H), 3.53 (m, 2H), 2.80 (s, 3H), 1.99 (s. 3H), MS (EI) for C₂₁H₂₀N₈O₃S: 465(MH⁺). MS(EI) for C₂₁H₂₀N₈O₃s: 465 (MH⁺).

6-(9-methyl-4-[6-methyl-5-(1-methylethyl)-2-(1-methylpyrrolidin-2-yl)pyrimidin-4- yl]-2,3,4,5-terahydro-1,4-benzoxazepin-7-yl}[1.3]thiazolo[5,4-b]pyridin-2-amine. ¹H NMR (400 MHz, d,i-DMSO): 8.35 (d, 1H), 7.87 (s.2H), 7.79 (d, 1H), 7.48 (d, 1H), 7.42 (d, 1H), 4.42 (s. 2H), 4.28 (m, 2H), 3.70(m, 2H), 3.26 (m. 1H), 3.20 (m. 2H), 2.98 (m 1H), 2.45 (s, 3H), 2.25 (s, 3H), 2.12 (s, 3H), 1.96 (m. 2H), 1.77 (m, 1H), 1.66 (m, 1H), 1.30 (dd, 6H ), MS (EI) for C₂₉H₃₅N₇OS: 531 (MH⁺). MS (EI) for C₂₉H₃₅N₇S: 53.1 (MH⁺).

6-{4-[2-cyclopropyl-6-metlhyl-5-(1-methylethyl)pyrimidin-4-yl]-9-methyl-2,3,4,5- tetrahydro-1,4-benzoxazepin-7-yl}[1,3]thiazol[5,4-b]pyridin-2amine. ¹H NMR (400, MHz, CD₃OD) δ 8.58 (s, 1H), 7.97 (d, 1H), 7.50 (s, 1H), 7.41 (s, 1H), 5.03 (s, 2H), 4.54 (m, 2H), 4.05 (t. 211), 3.20 (m. 1H), 2.58 (s, 3H), 2.22 (s,3H), 1.94 (in. 1H), 1.42(t, 6H), 1.01 (d. 2H), 0.82 (d, 2H), MS (ES) for C₂₇H₃₀N₆OS: 487 (MH⁺).

6-(4-(2-[(2S,4R)-4-fluoropyrroldin-2-yl]-6-methyl-5-(1-methylethyl)pyrimidin-4- yl}-9-methyl-2,3,4,5-tetrahydro-1,4-benzoxazepin-7-yl)[1,3]thiazolo-[5,4-b]pyridin-2-amine. ¹H NMR (400 MHz, CD₃OD) δ 8.36 (d, 1H), 7.83 (d, 1H), 7.41 (d, 2H), 5.17 (dt. 1H), 4.66 (s, 2H),4.42 (dd, 1H),4.37 (t, 2H), 3.85 (t, 2H), 3.34 (m, 1H), 3.18 (m, 2H), 2.54 (s. 3H), 2.44(m, 2H), 2.27 (s, 3H0 , 1.39 (d, 6H), MS (ES) for C₂₈H₃₂FN₇OS: 534 (MH⁺),

6-{9-methyl-4-[6-methyl-5-(1-methylethyl)-2-(methyloxy)pyrimidin-4-yl]-2,3,4,5- tetrahydro-1,4-benzoxazepin-7-yl}[1,3]thiazolo[5,4-b]pyridin-2-ainine. ¹H NMR (400 MHz. d₆-DMSO): 8.34 (t, 1H), 7.87 (s. 2H), 7.78 (t. 1H), 7.49 (d. 1H), 7.42 (d. 1H), 4.47 (s. 2H), 4.31 (s, 2H), 3.68 (s. 5H. overlapped), 3.25 -3.11 (m. 1H), 2.40 (s, 3H), 2.25 (s, 3H), 1.27. (t. 6H), MS (EI) for C₂₅H₂₈N₆O₂S: 477.2 (MH⁺).

6-(4-{2,6-dimethyl-[1-methyl-2-(methyloxy)ethyl]pyrimidin-4-yi }-9-methyl- 2,3,4,5-tetrahydro-1,4-benzoxazepin-7yl)[1,3]thiazolo[5,4-b]pyridin-2-amine. ¹H NMR (400MHz, d₆-DMSO): 8,37 (d. 1H), 7.89 (s. 2H): 7.81 (d, 1H), 7.52 (d, 1H),7.46 (d, 1H), 4.41 (d, 1H), 4.34 -4.10 (m, 3H), 3.71-3.63 (m,2H), 3.61 (d,2H), 3.53-3.40 (m, 1H), 3.20 (s. 3H), 2.41 (s, 3H), 2.39 (s, 3H), 2.28 (s, 3H), 1.23 (d. 3H), MS (EI) for C₂₆H₃₀N₆O₂S: 491(MH⁺).

6-{9-methyl-4-[6-methyl-5-(1-methylethyl)-2-{[2-(methyloxy)ethyl]oxy}pyrimidin- 4-yl]-2.3.4.5-tetrahydro- 1,4-benzoxazepin-7-yl][1,3]thiazolo[5,4-b]pyridin-2-amine. ¹H NMR (400 MHz, d₆-DMSO): 8.35 (d. 1H), 7.87 (s. 2H), 7.79 (d, 1H), 7.48 (s. 1H), 7.45 (d. 1:H), 4.49 (s, 2H), 4.31 (s, 2H), 4.18 -4.02 (mi 2H), 3.69 (s: 2H), 3.49 - 3.40 (m. 2H). 3.22 - 3.13 (m, 411, overlapped), 2.39 (s. 3H),2.24(s, 3H), 1.27 (t, 6H), MS (EI) for C₂₇H₃₂N₆O₃S: 521.2 (MH⁺).

6-(9-methyl-4-(6-methyl-5-(1-methylethyl)-2-[2-(methyloxy)ethyl]pyrimidin-4-yl]- 2,3,4,5-tetrahydro-1.4-benzoxazepin-7-yl)[1.3]thiazolo[5.4-b]pyridin-2-amine. ¹H NMR (400 MHz, d₆-DMSO): 8.37 (d, 1H), 7.92 (s. 2H), 7.79 (d. 1H), 7.52 (d. 1H), 7.44 (d, 1H), 4.42 (s, 2H), 4.28 (m. 2H), 3.68 (m. 2H), 3.63 (m, 2H), 3.24 (m. 1H), 3.12 (s. 3H), 2.79 (m 2H), 2.43 (s. 3H), 2.26 (s. 3H), 1.28 (d. 6H), MS (EI) for C₂₇H₃₂N₆O₂S: 505 (MH⁺),

6-{4-[2-{[(2-fluoroethyl)(metthyl)amino]methyl}-6-methyl-5-(1- methylethyl)pyrimidin-4-yl]-9-methyl-2,3,4,5-tetrahydro-1,4-benzoxazepin-7-

yl}[1,3]thiazolo[5.4-b]pyridin-2-amine. ¹H NMR (400MHz. d₄-MeOH): 8.33 (d, 1H), 7.79(d. 1H), 7.39 (s. 1H), 7.36 (s. 1H), 4.57 (tr. 1H), 4.54( s, 2H), 4.45 (tr, 1H), 4.34 (tr. 2H), 3.82(tr. 2H), 3.67 (d. 2H), 3.38 (m. 1H), 2.86 (m. 1H), 2.80 (m. 1H), 2.53 (s. 3H), 2.36 (d. 3H), 2.30 (s, 3H), 1.38 (d, 6H), MS (EI) for C₂₈H₃₄FN₇OS: 537 (MH⁺).

6-[4-[2-[(dimethylamino)methyl]-6-methyl-5-(1-methylethyl)pyrimidin-4-yl}-9- (methyloxy)-2,3,4,5-tetrahydro-1,4-benzoxazepin-7yl[1,3-thiazolo[5,4-b]pyridin-2-amine. ¹H NMR (400 MHz, DMSO-d₆) δ 8.37 (dd, 1H), 7.86 (s. 2H), 7.83 (d, 1H), 7.16 (dt. 2H), 4.39 (s, 2H), 4.20 (d. 2H), 3.84 (s, 3H), 3.63 (d. 2H), 3.37 (d, 2H), 3.25 (m. 1H), 2.44 (s, 3H), 2.15 (s, 6H), 1,30 (t, 6H), MS (EI) for C₂₇H₃₃N₇O₂S: 520.2 (MH⁺).

6- (4-{2-[(ethylamino)methyl]-6-methyl-5-(1-methylethyl)pyrimidin-4-yl}-9-methhyl- 2,3,4,5-tetrahydro-4-benzoxazepin-7-yl)[1,3]thiazolo[5.4-b]pyridin-2-amine. ¹H NMR (400 MHz.d₆-DMSO): 8.35 (d. 1H), 7.87 (s, 2H), 7.79 (d. 1H), 7.49 (d. 1H), 7.44 (d. 1H), 4.46 (s. 2H); 4.33-4.23-(m, 2H), 3.76-3.64 (m, 2H), 3.56 (s. 2H), 3.29-3.18 (m 1H), 2.46 (s, 3H); 2.43 (q. 2H), 2.25 (s, 3H), 1.89 (s, 3H), 1.31 (d,6H), 0.90.(t, 31H), MS (EI) for C₂₇H₃₃N₇OS: 504-(MH⁺).

6-( 4-[2-{[ethyl(2-fluoroethyl)amino]methyl}-6-methyl-5-(1-methylethyl)pyrimidin- 4-yl]-9-methyl-2,3,4,5-tetrahydro-1,4-benzoxazepin-7-yl}[1,3]thiazolo[5.4-b]pyridin-2- amine. ¹HNMR (400 MHz. d₄-MeOH): 8.32 (d. 1H), 7.79(d. 1H), 7.40 (s. 1H), 7.35(s. 1H), 4.54 (tr. 1H), 4.53 (s, 2H), 4.42 (tr, 1H), 4.34 (tr, 2H), 3.81 (tr, 2H), 3.77 (s, 2H), 3.38 (m, 1), 2.98 (tr. 1H), 2.91 (tr, 1H), 2.72 (q,2H), 2.53 (s, 3H), 2,29 (s, 3H), 1.38 (d, 6H), 1.04(tr. 3H), MS (EI) for C₂₉H₃₆FN₇OS: 551 (MH⁺),

N-[2-chloro-5-(9-methyl-4-{6-methyl-5-(1-methylethyl)-2-[2- (methyloxy)ethyl]pyrimidin-4-yl}-2,3,4-tetrahydro-1,4,-benzoxazepin-7yl)pyridin-3- yl]methanesulfulamide. ¹H NMR (400 MHz, d₆-DMSO): 9.89 (s, 1H), 8,51 (d. 1H), 8.02 (d, 1H), 7.54 (d, 1H), 7.44 (d, 1H), 4:45 (s. 2 H), 4.30 (m, 2H), 3:69 (in,2I-I), 3.60 (ni, 2H), 3.33(s. 3H), 3.24 (m, 1H), 3.12 (s. 3H), 2.79 (m, 2H), 2.44 (s. 3H), 2.24 (s, 3H), 1.27 (d, 6H), MS (EI) for C₂₇H₃₄ClN₅O₄S: 560 (MH⁺).

### EXAMPLE 2

### {4-[7-(2-Amino[1,3]thiazolo[5,4-b]pyridin-6-yl)-9-methyl-2,3-dihydro- 1,4-benzoxazepin-4(5H)-yl]-6-methyl-5-(1-methylethyl)pyrimidin-2-yl]methanol

STEP 1: A mixture of N-(6-bromo[3]thiazolo[5.4-b]pyridin-2-yl)acetamide (4.6 g, 17.0 mmol), bis(pinacolato)diboron (8.6 g, 39.0 mmol), bis(diphenylphosphino)ferrocene]dichloropalladium(II) (1.2 g. 1.75 mmol), and potassium acetate (0.25 g, 0.75.mmol) in 1,4-dioxane (50 mL) was degassed with nitrogen and heated at 130° C. in a microwave apparatus for 1 hour. The reaction mixture was cooled to room temperature and diluted with diethyl ether (100 mL). The solid was collected by. filtration. The crude filler cake was suspended in water, filtered and washed with hcxane to give N-[6- (4,4,5,5-tetramethyl-1,3,2-dioxaborolan-2-yl)[1,3]yhiazolo[5.4-b]pyridin-2-yl]acetamide as light brown solid (2.8 g. 52%), ^{‘}H NMR (400 MHz. DMSO-d₆): 12.60 (s. 1H), 8.60 (s. 1H), 17 (s. 1H), 2.19 (s. 3H), 1.29 (s. 12H); MS (EI) for C₁₄H₁₈BrN₃O₃S: 320 (MH⁺).

STEP 2: A mixtiue of 2-(chloromethyl)-6-methyl-5-(1-methylethyl)pyrimidin-4- ol(1.20 g, 6.00 mmol), cesium-acctate (11.46 g. 60 mmpl) in glacial acetic acid (20 mL) was heated at 130° C. in a microwave apparatus for 1 hour. After cooling to room temperature the reaction mixture was partitioned between water and ethyl acetate. The organic layer was separated washed with saturated aqueous sodium hydrogen carbonate, brine, dried over anhydrous magnesium sulfate, filtered, and concentrated to give [4-hydroxy-6-meihyl-5-(1- methylethyl)pyrimidin-2-yl]methy acetate which was used without further purification in the next step. (1.3 g, quant.), MS (EI) for C₁₁H₁₆N₂O₃: 225 (MH⁺),

STEP 3.: A solution of [4-hydroxy-6-imethyl-5-(1-methylethyl)pyrimidin-2- yl]methyl acetate:(1.3 g. 6.00 nimol), prepared in step 2, in phosphorus oxychloride (8.0 mL, 85.84 mmol) was heated to reflux for 1 hour. After cooling to room temperature, the reaction mixture was concentrated and the resulting residue was partitioned between saturated aqueous sodium hydrogen carbonate and ethyl acetate. The organic layer wasseparated. washed with brine, dried over anhydrous magnesium sulfate, filtered and concentrated. The residue was purified by gradient silica gel chromatography (hexane:ethyl acetate 9:1 to 1:1) to provide [4-chloro-6-methyl-5-(1-methylethyl)pyrimidin-2-yl methyl acetate. MS (EI) for C₁₁H₁₅ClN₂O₂: 243 (MM⁺).

STEP 4: A solution of 1.1 -dimeihylethyl 7-bromo-9-methl-2,3-dihydro-1.4- bcnzoxazepine-4(5H)-carboxylate (9.0 g. 26.3 mmol) in a mixture of methanol (30 mL) and 4N hydrogen chloride in 1,4-dioxane (10 mL) was refluxed for 30 minutes. After cooling to room temperature, the reaction mixture was concentrated to a reduced volume and the precipitate that formed was collected by filtration washed several times with ethyl acetate and hexanes then dried in vacuo to give 7-bromo-9-methyl-2,3,4,5-tetrahydro-1,4- benzoxazepine hydrochloride (5.7 g. 78%) as a while solid. ¹H NMR (400 MHz. DMSO-d₆): 9.57 (br.s. 1H), 7.52 (d. 1H), 7.47 (d. 1H), 4.26 (s, 2H), 4.18 (m, 2H), 3.43 (m. 2H), 2.19 (s, 3H); MS (EI),for C₁₀H₁₂BrNO: 243 (MH⁺).

STEP 5: A mixture of 7-bromo-9-methyl-2,3,4,5-tetrahydro-1.4-benzoxazepine hydrochloride (0,82 g, 2.90 mmol), [4-chloro-6-methyl-5-(1-methylethyl)pyrimidin-2- yl]methyl acetate (0.70 g. 2.90 mmol) prepared in step 3and N,N-diisppropylethylaniine (1.9mL, 10.9 mmpl) in N,N-dimethylacetamide (3.0 mL) was healed at 130° C. for 3 hours. After cooling to room temperature, the reaction mixture was partilioned between water and dichloromethane. The organic layer was separated washed wilh brine, dried over anhydrous sodium sulfate, filtered and concentrated. Gradient silica gel chromatography oil the residue (hexane:ethyl acetate 9.1 to 1:1) provided [4-(7-bromo-9-methyl-2,3-dihydro-1,4-

benzoxazepin-4(5H)-yl)6-methyl-5-(1-methylethyl)pyrimidin-2yl]methyl acetate (0.65 g. 50%), MS (EI) for C₂₁H₂₆BrN₃O₃: 448 (MH⁺),

STEP 6: A mixture of [4-(7-bromo-9-methyl-2.3-dihydro-l,4-benzoxazepin- 4(5H)-yl)-6-methy-5-(1 -methylethyl)pyrimidin-2-yl]meth yl acetate (68 mg, 0.15 mmol) prepared in step 5, N- 6-(4,4,5.5-tetramethyl-1,3,2-dioxaborolan-2-yl)[1,3]thiazolo[5,4- b]pyridin-2-yl]acetamide (48 mg, 0.15 mmol) prepared in step 1. bis(diphenyphosino)ferrocene]dichloropalladium(II) (48 mg, 0.075 ) and cesium carbonate (0.25g, 0.75 mmol) in a mixtureof 1,4-dioxane (2.6 mL) and water (0.4 mL) was heated at 130° C. using a micrwave apparatus for 2 hours. The reaction mixture was cooled to room temperature and partitioned between ethyl aceiateand saturated aqueous sodium hydrogen carbonate. The organic layer was separated washed with brine, dried over anhydrous magnesium sulfate, filtered and concentrated. Silica gel chromatography of the reside (chloroform : methanol 95:5) provided {4-7-(2-amino[1.3]thiazolo[5.4-b]pyridin-6- yl)-9-methyl-2.3-dihydro-1,4-benzoxazepin-4(5H)-yl]6-methyl-5(1-methylethyl)pyrimidin- 2-yl (methanol (24 mg. 34%), ¹NMR (400 MHz. CD₃OD) ε 8.56 (d, 1H), 7.95 (d, 1H), 7.56(d. 1H), 7.45 (d. 1H), 5.05 (s. 2H), 4.56 (s. 2H), 4.48 (m, 2H), 4.15 (m, 2H), 3.21 (m. 1H), 2.63 (s, 3H), 2.27 (s. 3H), 1,43 (d. 6H); MS (ES) for C₂₅H₂₈N₆O₂S: 477 (MH⁺).

Proceeding according to the method of Example 2 and replacing [4-(7-bromo-9- methyl-2.3-dihydro-1.4-benzoxazepin-4(5H)-yl)-6-methyl-5-(1-melhylethyl)pyrimidiim-2- yl]methyl acetate in step 6 with 1-[4-(7-bromo-9-methyl-2,3-dihydro-1,4-benzoxazepin- 4(5H)-yl)-5-isopropyl-6-methylpyrimidin-2-yl]ethanol, the following compound of the invention was prepared:

1-{4-[7-(2-Amino[1m3]thiazolo[5,4-b]pyridin-6-yl)-9-methyl-2,3-cdihydro-4- benzbxazepin-4(5H)-yl]-6-methylo-(1-methylethyl)pyrimidin-2-yl (ethanol. ¹H NMR (400MHz. DMSO-dr,): 8.36 (d. 1H), 7,88 (s. 2H), 7.80 (d. 1H), 7.49 (d. 1H), 7.45 (d. 1H), 4.70(br.s. 1H), 4.48 (m. 1H), 4.47 (s. 2H), 4.30 (m. 2H)), 3.72 (m, 2H), 3.26 (m. 1H), 2.49 (s, 3H), 2.24 (s. 3H), 1,32 (dd, 3H), 1.26 (d, 6H), MS (EI) for C₂₆H₃₀N₆O₂S 491 (MH⁺).

Proceeding according to the method of Example 2 and replacing [4-(7-bromo-9- methyl-2,3-dihydro-1,4-benzoxazepin-4(5H)-yl)-6-methyl-5-(1-methylethyl)pyrimidin-2- yl]methyl acctale in sicp 6 with 1-[4-(7-bromo-9-methyl-2,3-dihydro-1,4-benzoxazepin- 4(5H)-yl)-5-isopropyl-6-methylpyrimidin-2-yl]-2,2,2-trifluoroethanol, the following compound of the invenlion was prepared:

1-[4-[7-(2-Amino[1,3]thiazolo[5,4-b]pyridin-6-yl)-9-methyl-2,3-dihydro-1,4- benzoxazepin-4(5H)-yl]-6-methyl-5-(1-methylethyl)pyrimidin-2-yl]-2,2,2-trifluorethanol. ¹H NMR (400 MHz. DMSO-d₆): 8.35 (d, 1H), 7.88 (s,. 2H), 7.80 (e, 1H), 7.49 (d, 1H), 7.42(cl, 1H), 6.57 (br. s, 1H), 4.88 (m, 1H), 4.51 (s. 2H), 4.32 (m. 2H), 3.74 (m, 2H), 3.26 (m, 1H), 2.52 (s, 3H),2.25 (s,3H), 1.47 (t, 6H); MS (EI) for C₂₆H₂₇F₃N₆O₂S. 543 (MH⁺),

1-{4-[7-(2-amino[3]thiazolo[5.4-b]pyridin-6-yl)-9-methyl-2,3-dihydro-1.4- benzoxazepin-4(5H)-yl]-6-methyl-5-(1-methylethyl)pyrimidin-2-yl]ethanol. ¹H NMR (400MHz, d₆-DMSO): 8.36 (d, 1H), 7.87 (s. 2H), 7.80 (d, 1H), 7.49 (d, 1H), 7.45 (d, 1H),4.72(brs, 1H), 4.50 (m, 1H), 4.47 (s, 2H), 4.30 (m. 2H), 3.76 (m. 2H), 3.24 (m, 1H), 2.46 (s, 3H), 2.25 (s, 3H), 1.32 (dd, 3H), 1:24 (d, 6H), MS (Ei) for C₂₆H₃₀N₆O₂S: 491 (MH⁺),

6-{4-[2-amino-6-mcethyl-5-(1-methylethyl)pyrimidin-4yl]-9-(methoxy)-2,3,4,5- tetrahydro-1,4-benzoxazepin-7-yl}[3]thiazolo[5,4-b]pyridin-2-amine. ¹H NMR (400 MHz. DMSO-d₆) 8.33 (s. 1H), 7.79(s. 3H), 7.20 (s, 1H), 7.07 (s, 1H), 6.24 (bs. 2H), 4.30 (s, 2H), . 4.18 (s, 2H), 3.80 (s, 3H), 3.54 (s. 2H), 3.09 (m, 1H), 2.24 (s, 3H), 1.18 (d, 6H), MS (EI) for C₂₄H₂₇N₇O₂S: 478.2 (MH⁺),

### EXAMPLE 3

### N,N-Dimethyl-1-{4-methyl-5-(1-methylethyl)-6-[9-methyl-7-(2-methyl-1H-benzimidazol- 6-yl)-2,3-dihydro1,4-benzoxazepin-4(5H)-yl]pyrimidin-2-yl}methanamine

STEP 1: A mixture of 7-bromo-9-methyl-2,3.4,5-tetrahydro-1,4-benzoxazepine hydrochloride (1.0 g, 3.61 mmol), 1-[4-chloro-6-methyl-5-(1-methylethyl)pyrimdin-2-yl]- N,N-dimethylmethanamine (0:82 g, 3.61 mmol), and N,N-diisopropyiethylamine (3.1 mL, 18.1 mmol) in N,N-dimethylacetamide (5.0 mL) was heated at 130° C. for 3 hours. After cooling to room temperature, the reaction mixture was partitioned between water and ethyl acetate. The organic layer was separated washed with brine (2 x 100 mL), dried over anhydrous sodium sulfate, filtered and concentrated. Gradient silica gel chromatography of the residue (chloroform : methanol 95:5to 9:1) provided 1-[4-(7-bromo-9-methyl-2.3- dihydro- 1,4-benzoxazepin-4(5H)-yl)-6-methyl-5-(1-methylethyl)pyrimidin-2-yl]-N,N- dimethylmethanamine (0.64 g, 41%), MS (EI) for C₂₁H₂₉BrN₄O: 433 (MH⁺),

STEP 2: A mixture of ]-[4-(7-bromo-9-methyl-2,3-dihydro-1,4-benzoxazepin- 4(5H)-yl)-6-methyl-5-(1-methylethyl)pyrimidin-2-yl]-N,N-dimethylmethanamine (90 mg, 0.21 mmol), (1-[{[(1,1-dimethylethyl)oxy]carbonyl}-2-methyl-1H-benzimidazol-6-yl)boronic acid (60 mg, 0.22.mmol), bis(diphenylphqsphino)ferrocenc)dichlpropalladium (II) (32 mg, 0.042 mmol) and cesium carbonate (0.36 g, 1.10 mmol) in a mixture of 1,4-dioxane (2.7 mL), and water (0.3 niL) was healed at 130° C. using it microwave apparatus for 1 hour. The reaction mixture was cooled to room temperature and partitioned between water and ethyl acetate. The organic layer was separated washed with brine, dried over anhydrous magnesium sulfate, filtered, and concentrated. The resulting residue was dissolved in methanol (5.0) mL) and concentrated hydrochloric acid (0.5mL) was added to the solution. The reaction mixture was heated to reflux, for 5 minutes. After cooling to room temperature, the mixture was concentrated and the residue purified by preparative reverse phase HPLC (0.1% aqueous ammonium acetate buffered aqueous acetonitrile mobile phase) to give N,N-dimethyl-1-{4- methyl-5(1--methylethyl)-6-[9-methyl-7-(2-methyl-1H-benimidazol-6-yl)-2.3-dihydro-1,4- benzoxazepin-4(5H)-yl]pyrimidin-2-yl}methananamine (42 mg. 42%), ¹HNMR (400 MHz. DMSO-d₆): 7,62 (s, 1H), 7.49 (d, 1 H), 7.41 (d. 1H), 7.35 (dd. 1H), 7.34 (s. 1H),7,30 (br.s. 1H), 4.37(s. 2H), 4.26 (s, 2H), 3.66 (s, 2H), 3.38 (s. 2H), 3.33 (m, 1H), 2.47 (s. 3H), 2.26 (s, 3H), 2.16 (s, 6H), 1.86 (s, 3H), 1,32 (d, 6H): MS (EI) for C₂₉H₃₆N₆O: 485 (MH⁺),

Proceeding according lo the method of Example 3 and replacing 1-[4-chloro-6- methyl-5-(1-methylethyl)pyrimidin-2-yl]-N,N-dimethylmethanainine in step 1 with 4-chloro- 5-isopropyl-6-methylpyrimidin-2-amine and replacing (1-{[(1.1- dimethylethyl)oxy]carbonyl]-2-methyl-1H-benzimidazol-6-yl)boronic acid in step 2 with alternative reagents, the following compounds of the invention eere prepared:

Methyl 4-{4-[2-Amino-6-methyl-5-(1-methylethyl)pyrimidin-4yl]-9-methyl-2,3,4,5- tetrahydro-1,4-benzoxazepin-7-yl]-2-(methyloxy)benzoaic. ¹H NMR (400 MHz. d₆-DMSO) δ 7.73 (d, 1H), 7.55 (d, 1H), 7.43 (d, 1H), 7.3.1 (d, 1H), 7,26 (dd, 1H), 6.05 (s. 2H), 4.25 (s, 4H), 3.91 (s, 3H), 3.79 (s, 3H), 3.53 (s, 3H), 3.19 (dd, 1H), 2.29 (d, 6H), 1.24 (d, 6H), MS (EI) for C₂₇H₃₂N₄O_{4:} : 477.11 (MM⁺),

4-[7-(3-Aminophenyl)-9-metyl-2,3-dihydro-1,4-benzoxazepin-4(5H)-yl)-6-methyl- 5-(1-methylethyl)pyrimidin-2-amine. ¹H NMR (400 MHz, d₆-DMSO) δ 7.30 (s, 1H), 7.20 (d. 1H), 7.06 (t, 1H), 6.77 (d. 1H), 6.70 (d, 1H), 6.51 (dd, 1H), 6.01 (s. 2H), 4.20 (d. 4H), 3.53 (s. 2H), 3.24-3.16 (m. 1H), 2.30 (s. 3H), 2.25 (s, 3H), 1.25 (d. 6H), MS (EI) for C₂₄H₂₉N₅O: 404.13 (MH⁺).

3-{4-[2-Amino-6-methyl-5-(1-methylethyl)pyrimidin-4-yl]-9-meithyl-2,3,4,5- tetrahydro-1,4-benzoxazepin-7-yl}phenol. ¹H NMR (400 MHz, d₆-DMSQ) δ 7.35 (s. 1H), 7.26 - 7.18 (m. 2H), 7.00 (d. 1H), 6.95 (d, 1H), 6.72 (dd, 1H), 6.02 (s. 2H), 4.21 (d. 4H), 3.54(s, 2H), 3.23 (dd, 1H), 2.30 (s. 3H), 2.26 (s, 3H), 1.26 (d,6H), MS (EI) for C₂₄H₂₈N₄O₂:405.08 (MH⁺).

4-Methyl-5-(1-methylethyl)-6-(9-methyl-7-pyrimidin-5-yl-2,3-dihydro-1,4- benzoxazexpin-4(5H)-yl)pyrimidih-2-amine. ¹H NMR (400 MHz, d₆-DMSO) δ 9.14 (s, 1H), 9.08 (s. 2H), 7.60 (d, 1H), 7.48 (d. 1H), 6.02 (s, 2H), 4.28 (s, 4H), 3.53 (s, 2H), 3.21 - 3.11(m, 1H), 2.28 (d, 6H), 1.23 (d, 6H), MS (EI) for C₂₂H₂₆N₆O: 391.10 (MH⁺),

4-Methyl-5-(1-methylethyl)-6-(9-methyl-7-(1H-pyrazol-5-yl)-2,3-dihydro-1,4, bcnzbxazoxepin-4(5H)-yl]pyrimidin-2-amine. ¹H NMR; (400 MHz, d₆-DMSO) 5.8.29 (s, 1H), 7.67 (s. 1H), 7.54 (s. 1H), 7.49 (s, 1H), 6.59 (d, 1H), 6.01 (s, 2H), 4.20 (s, 4H); 3.52 (s, 2H), 3.25-3.13 (m, 1H), 2.29 (s, 3H), 2,24 (s. 3H), 1.24 (d, 6H),MS (EI) for C₂₁H₂₆N₆O: 379.08 (MH⁺).

4-[7-(1,3-Benzodioxol-5-yl)-9-methyl-2,3-dihydro-1,4-benzoxazepin-4(5H)-yl]-6- methyl-(1-methylethyl)pyrimidin-2-anine. ¹H NMR (400 MHz, d₆-DMSO) δ]7.36 (d, 1H), 7.24 (d, 1H), 7.16 (d, 1H), 7.07 (dd. 1H), 6.98 (d, 1H), 6.05 (s, 2H), 6.02 (s, 1H), 4.19 (s, 4H), 3.52 (s. 2H), 3.25 - 3.15 (m, 211), 2.29 (s. 3H),2.25(s. 3H), 1.24 (d.6H), MS (EI) for C₂₅H₂₈N₄O₃: 433.09 (MH⁺),

4-Methyl -5-(1-methylethyl)-6-(9-methyl-7-pyridin-4-yl-2,3-dihydro-1,4- benzoxazepin-4(5H)-yl)pyrimidin-2-amine. ¹H NMR (400 MHz. D₆-DMSO): δ 8.60 (dd. 2H), 7.63 (d, 2H),7.48 (s, 1H),7.60(s, 1H), 6.02 (s, 2H), 4.26 (s,4H), 3.45 (m, 2H), 3.17 (m, 1H), 2.29.(s. 6H), 1.23 (d, 6H); MS (EI)for C₂₃H₂₇N_{5O found} 390.09 (MH⁺).

4-Methyl-5-(1-methylethyl)-6-(9-methyl-7-pyridin-3-yl-2,3-diliydro-1,4- benzoxazepin-4(5H)-yl)pyrimidin-2-amine. ¹H NMR (400 MHz, D₆-DMSO): δ 8.84 (d, 1H), 8.53 (dd, 1H), 8.01 (m, III), 7.48 (m, 1H), 7:42 (in, 1H), 7.39 (d, 1H), 6.02 (s, 2H), 4.25 (s, 4H), 3.60 (s, 2H), 3.20 (q, 1H), 2.29 (m. 6H), 1.24 (cl, 6H); MS (EI) for C₂₃H₂₇N₅O. found390.09 (MH⁺).

3-(4- 8 2-Amino-6-methy-1-5-(1- iiiethy I etliy Ii)pyr im id i n-4-y 11-9-met Hy 1-2,3tetrahydro-1,4-benzoxazepin-7-yl}benzamide. ¹H NMR (400 MHz, D₆-DMSO): δ 8.09 (d. 2H), 7.78 (dd, 2H), 7.52 (l, 2H), 7.46 - 7.25 (m,2H), 6.02 (s, 2H), 4:25 (s, 4H), 3.53 (s, 2H), 3.21 (q, 1H), 2.29 (s, 6H), 1.24 (d, 6H); MS (EI) for C₂₅H₂₉N₂₉N₅O₂. found 432.09 (MH⁺).

4-{7-[3.4-Bis(methyloxy)phenyl]-9-methyl-2,3-dihydro-1,4-benzoxazepin-4(5H)-yl}- 6-methyl-5-(1-methylethyl)pyrimidin-2-amine. ¹H NMR (400MHz, D₆-DMSO): δ 7.41 (d, 1H), 7.29 (d, 1H), 7.13 (m, 2H), 7.02 (d, 1H), (s, 6.02(s, 2H), 4.19 (s, 4H), 3.83 (s, 3H), 3.78 (s. 3H), 3.52 (s, 2H), 3.24 (q, 1H), 2.30 (s, 3H), 2.26 (s, 3H), 1.26 (d, 6H); MS (EI) for C₂₆H₃₂N₄O₃, found 449.09 (MH⁺).

4-Methyl-5-(1-methylethyl)-6-(9-methyl-7-[5-(methyloxy)pyridin-3-yl]-2,3-dihydro- 1.4-benzoxazepin-4(5H)-yl}pyrimidin-2-amine. ¹H NMR (400 MHz, D₆-DMO): δ 8.43 (d. 1H), 8.25 (d. H), 7.55 (m. 2H), 7.41 (d, 1H), 6.03 (s,.2H), 4.24 (s, 4H), 3.90 (s. 3H), 3.53(m, 2H), 3.20 (q, 1H), 2.29 (s, 3H), 2.28 (s, 3H), 1.24 (d, 6H); MS (EI) for C₂₄H₂₉N₅O₂, found 420.12 (MH⁺).

4-Methyl-5-(1-methylethyl)-6-[9-methyl-7-(1H-pyrazol-4-yl)-2,3-dihydro-1,4- benzoxazepin-4(5H)-yl]pyrimidin-2-amine. ¹H NMR (400 MHz, D₆-DMSO): 8 8.34 (s, 1H), 7.93 (s, 2H), 7.35 (s, 1H), 7.25 (s, 1H), 6.02 (s, 2H), 4.17 (s, 4H), 3.49 (s. 2H), 3.19 (q, 1H), 2.29 (s, 3H), 2.21 (s. 3H), 1.23 (d. 6H); MS (EI) for C₂₁H₂₆N₆O. found 379.14 (MH⁺).

4-[7-(2-Aminopyrimidin-5-yl)-9-methyl-2.3-dihydro-1,4-benzoxazepin-4(5H)-yl]-6- methyl-5-(1-methylethyl)pyrimidin-2-amine. ¹H NMR (400 MHz, D₆-DMSO): δ 8.50 (s. 2H), 7.38 (s. 1H), 7.25 (s, 1 H), 6.72 (s, 2H), 5.99 (s, 2H), 420 (s, 4H), 3.51,(s. 2H); 3.19 (q. 1H), 2.29 (s. 3H), 2.24 (s, 3H), 1.23 (d, 6H); MS (EI) for C₂₂H₂₇N₂₇N₇O found 406.08 (MH⁺).

4-Methyl-5-(1-methylethyl)-6-[9-methyl-7-[2-(methyloxy)pyrimidin-5-yl]-2,3- dihydro-4-benzoxazepin-4-(5H)-yl}pyrimidin-2-amine. ¹H NMR (400 MHz, D₆-DMSO): δ 8.86 (s. 2H), 7.50(s, 1H), 7.37 (s, 1H), 6.02 (s, 2H), 4.25 (s, 4H); 3.95 (s. 3H), 3.52 (s. 2H), 3.17 (q, 1H), 2.28 (s. 3H), 2.26(s, 3H), 1.23 (d. 6H): MS (EI) for C₂₃H₂₈N₆O₂, found 420.51 (MH⁺).

4-[7-(2-Fluoropyridin-4-yl)-9-methyl-2,3-dihydro-1,4-benzoxazepin-4(5H)-yl]-6- methyl-5-(1-methylethyl)pyrimidin-2-amine. ¹HNMR (400 MHz, D₆-DMSO): δ 8.27 (d. 1H), 7.66 (m, 2H), 7.56 (d, 1H), 7.46 (s. 1H), 6.01 (s, 2H), 4.29 (s.4H), 3.53 (s, 2H), 3.14 (q, 1H), 228 (s. 6H), 1.89 (s, 1H), 1.22 (d, 6M): MS (EI) for C₂₃H₂₆FN₅O. found408.51 (MH⁺).

4-[7-(2-Amino-1,3-thiazo-5-yl)-9-methyl-2,3-dihydro-1,4-benzoxazepin-4(5H)-yl]-6- methyl-5-(1-methylethyl)pyrimidin-2-amine. ¹H NMR (400 MHz. D₆-DMSO): δ 7.24 (m. 2H), 7.06 (m, 3H); 6.01 (s, 2H), 4.17(m. 2H),4.11 (s. 2H), 3.51 (s. 2H), 3.19 (q, 1H); 2.30 (s. 3H), 2.20 (s, 3H), 1.25 (d, 6H); MS (EI) for C₂₁H₂₆N₆OS. found 411.50 (MH⁺).

4-[7-(6-Aminopyridin-3-yl)-9-methyl-2,3-dibydro-1,4-benzoxazepin-4(5H)-yl]-6- methyl-5-(1-methylethyl)pyrimidin-2-amine. ¹H NMR (400 MHz, methanol-d₆): 8.09 (d, 1H), 7.71 (dd, 1H), 7.25 (m, 2H), 6.66,(d. 1H), 4.47 (s. 2H), 429 (m. 2H), 3,78.(m. 2H), 3.24 (m, 1H), 2.39 (s, 3H), 2.28 (s. 3H), 1.95 (s. 3H), 1.32 (d. 6H); MS (EI) for C₂₃H₂₈N₆O: 405(MH⁺).

4-Methyl-5-(1-methlethyl)-6-{9-methyl-7-[6-(methyloxy)pyridin-3-yl]-2.3-dihydro- 1,4-benzoxazepin-4(5H)-yl}pyrimidin-2-amine. ¹H NMR (400 MHz, d₆-DMSO) δ 8.40 (d. 1H), 7.94 (dd, 1H), 7.42 (s. 1H), 7.30 (s. 1H), 6.90 (d, 1H), 6.02 (s. 2H), 4.22 (s. 4H), 3.88 (s. 3H), 3.53 (s, 2H), 3.19 (dd, 1H), 2.28 (d. 6H), 1.24 (d, 6H), MS (EI) for C₂₄H₂₉N₅O₂: 420.12 (MH⁺).

Proceeding accdrdiiig to the method of Example 3 and replacing 1-[4-chloro-6- methyl-5-(1-methylethyl)pyrimidin-2-yl]-N,N-dimethylmethanamine in step 1 with 4-chloro- 5-isopropyl-6-methylpyrimidin-2-amine the following compound of the invention was prepared:

4-Methyl-5-(1-methylethyl)-6-[9-methyl-7-(2-methyl-1 H-benzimidazol-6-yl)-2,3- dihydro-1,4-benzoxasepin-4(5H)-yl]pyrimidin-25amine. ¹H NMR (400 MHz, d₆-DMSO) δ 7.66 (s. 1H), 7.50 (s, 1H), 7.43 (d, 1H), 7.35 (d. H), 7.31 (s. 1H), 6.02 (s, 2H), 4.21 (s, 4H), 3.54(m. 2H), 3.26 (m. 1H), 2.50 (s. 3H), 2.30 (s, 3H). 2.28 (s, 3H), 1.27 (d, 6H): MS (ES) for C₂₆H₃₀N₆O: 443 (MH⁺).

4-[7-(1,3-dimethy-1H-pyrazol-4-yl)-9-methyl-2,3-dihydro-1,4-benzoxazepin-4(5H)- yl]-6-methyl-5-(1-methylethyl)pyrimidin-2-amine. ¹H NMR (400 MHz, d₆-DMSO): 7.76 (s, 1H), 7.14 (s. 1H), 7.03 (s. 1H), 6.00 (s, 2H), 4.19 (br tr. 2H), 4.15 (s, 2H), 3.76 (s. 3H), 3.51(br tr. 2H), 3.23 (m. 1H), 2.29 (s. 3H), 2.24 (s, 3H),2.22 (s. 2H), 1.25 (d. 6H), MS (EI) for C₂₃H₃₀N₆O: 408 (MH⁺).

4-[7-(1,5-dimethy-1H-pyrazol-4-yl)-9-methyl-2,3-dihydro-1,4-benzoxazepin-4(5H)- yl]-6-methyl-5-(1-methylethyl)pyrimidin-2-amine. ¹H NMR (400MHz, d₆-DMO). 7.47 (s. 1H), 7.13 (s, 1H), 7.00 (s. 1H), 6.00 (s. 2H), 4.20 (br tr. 2H), 4.15 (s, 2H), 3.76 (s. 3H), 3.52(br tr. 2H), 3.24 (m. 1H); 2.33 (s. 3H), 2.29 (s, 3H), 2.23 (s. 3H), 1.25 (d. 6H), MS (EI) for C₂₃H₃₀N₆O: 408 (MH⁺).

4-[1-(1-ethyl-1H-4-yl)-9-methyl-2,3-dihydro-1,4-benzoxazepin-4(5H)-yl]-6- methyl-5-(1-melhylethyl)pyrimidin-2-amine. ¹H NMR (400 MHz, d₆-DMSO): 8.06 (s, 1H), 7.74 (s. 1H), 7.32 (s. 1H), 7.21 (s. 1H), 6.01 (s. 2H), 4.17 (br s, 4H), 4.13 (q, 2H), 3.49 (br tr. 2H), 3.19 (m. 1H), 2.29 (s. 3H), 2.21 (s. 3H), 1.38 (tr. 3H), 1.24 (d, 6H), MS (EI) for C₂₃H₃₀N₆O: 408 (MH⁺).

4-methyl-5-(1-methylethyl)-6-{9-methyl-7-[2-(methylamino)-1,3-thiazol-4-yl]-2,3- dihydro-1,4-benzoxazepin-4(5H)-yl}pyrimidin-2-amine. ¹H NMR (400 MHz. d₆-DMSO): 7.78 (s, 1 H),.7.69 (s, 1H), 7.63 (s. 1H), 6.00 (s, 2H), 4.22 (br s, 411), 4.03 (s, 3H), 3.52 (br tr, 2H), 3.18 (m, 1H), 2.294 (s, 3H), 2.25 (s. 3H), 1.24(d. 6H), MS (EI) for C₂₂H₂₈N₆OS: 426 (MH⁺).

4-methyl-5-(1-methylethyl)-6-[9-methyl-7-(1 -methyl-1H-pyrazol-4-yl)-2.3-dihydro- 1,4-benzoxazepin-4(5H)-yl]pyrimidin-2-amine. ¹H NMR (400 MHz. d₆-DMSO): 8.01 (s, 1H), 7.74 (s, l.H), 7.31 (s. 111), 7.20 (s, 1H), 6.07 (br s. 21-1), 4.20(br s. 411), 3.85 (s, 3H), 3.52 (brs, 3H), 3.17 (m. 1H), 2.30 (s, 3)H, 2.21 (s. 3H), 1.24 (d, 6H), MS (EI) for C₂₂H₂₈N₆O: 394 (MH⁺).

4-methyl-5-(1-methylethyl)-6-[9-methyl-7-(2-methyl-1,3-thiazol-5-yl-dihydro- 1,4-benzoxazepin-4(5H)-yl]pyrimidim-2-amine. ¹HNMR (400 MHz, d₆-DMSO): 7.90 (s, 1H), 7;42 (s. 1H), 7.25 (s, 1H), 6.00 (,s, 1H),4.22 (br tr, 2H), 4.18(s, 2H), 3.52 (s, 2H), 3.16(m. 1H), 2.66 (s. 3H), 2.29 (s. 3H), 2.23 (s. 3H), 1.24 (d, 6H), MS (EI) for C₂₂H₂₇N₅OS: 411 (MH⁺).

N-([4-[7-(2-amiuo[1,3]thiazolo[5,4-b]pyridin-6-yl)-9-methyl-2,3-dihydro-1,4- benzoxazepin-4(5H)-yl]-6-methyl-5-(1-methylethyl)pyrimidin-2-yl]methyl)acetamide. ¹H NMR (400MHz. d₆-DMSO) δ 8.35 (d. 1H), 8.08 (t. 1H), 7.87 (s. 1H), 7.79 (d. 1H), 7.51 (s. 1H), 7.40 (s. 1H), 4.41 (s. 2H), 4.30 (m. 2H), 4.19 (d, 2H), 3.70 (m. 2H), 3.26 (m, 1H), 2.47(s, 3H), 2.27 (s, 3H), 1,85 (s. 3H), 1.31 (d, 6H), MS (ES) for C₂₇H₃₁N₇O₂S: 518(MH⁺),

6-[4-[2-(fluoromethyl)-6-methyl-5-(1-methylethyl)pyrimidin-4-yl]-9-methyl-2,3,4,5- tetrahydro-1,4-benzoxazepin-7-yl}[1.3]thiazolo[5.4-b]pyimidin-2-amine. ¹H NMR (400 MHz, CD₃OD) δ 8.61 (d, 1H), 7,98 (d, 1H), 7.56 (s. 1H), 7.46 (s, 1H); 5.40 (d, 2H), 5.08 (s. 2H), 4.50(m. 2H), 4.17 (m. 2H), 3.22 (m. 1H), 2.64 (s, 3H), 2.27 (s. 3H), 1.44 (d, 6H), MS (ES) for C₂₅H₂₇FN₆OS: 479 (MH⁺).

6-[4-{2-[(dimethylamino)6-methyl-5-(1-methylethyl)pyrimidin-4-yl}-9- (methyloxy)-2.3.4.5-tetrahydro-1,4-benzpxazepin-7-yl][1,3]thiazolo[5,4-b]pyridin-2-amine. ¹H NMR (400 MHz, DMSO-d₆) δ 8.37 (dd, 1H), 7.86(s. 2H), 7.83 (d, 1H), 7.16 (dt. 2H), 4.39 (s,.2H), 4.20 (d. 2H), 3.84 (s, 3H), 3.63 (d. 2H), 3.37 (d. 2H), 3.25 (m. 1H), 2.44 (s. 3H), 2.15 (s, 6H), 1.30(t, 6H), MS(EI) for C₂₇H₃₃N₇O₂S: 520.2 (MH⁺).

4-methyl-5-(1-methylethyl)-6-[9-methyl-7-(2-methyl-3H-imidazo[4,5-b]pyridin-6- yl)-2,3-dihydro-1,4-benzoxazepin-4(5H)-yl]pyrimidin-2-amine. ¹H NMR (400 MHz, d₆- DMSO): 8.48(br s. 1H), 8.00 (brs, 1H), 7.50 (d. 1H), 7.36 (d. 1H), 6.02 (s, 2H), 4.30-4.17 (m. 4H), 3.58-3.48 (m, 2H), 3.33 (s, 3H), 3.29-3.19 (m, 1H), 2.53 (s. 3H), 2.29 (s. 3H), 2.29(s. 3H), 1.26 (d. 6H), MS (EI) for C₂₅H₂₉N₇O: 444 (MH⁺).

4-[7-(1H-imidazo[4.5-b]pyridin-6-yl)-9-methyl-2,3-dihydro-1,4-bcnzoxazcpin-4)5H)- yl]-6-methyl-5-(1-methylethyl)pyrimidin-2-amine. ¹H NMR (400 MHz: methanol-d₄): 8.61(t, 1H), 8.41 (d. 1H), 8.16 (t, 1H), 7.44 (d, 1H), 7.37 (d.. 1H), 4.37 (s. 2H), 4.28 (m. 2H),3.68(m, 2H), 3.38 (m, 1H), 2.38 (s, 3), 2.33 (s. 3H), 1.33 (d. 6H), MS (EI) for C₂₄H₂₇N₇O: 430.1 (MH⁺), N-(2-chloro-5-{4-[2-{[(2.2-difluoroethyl)amino]methyl}-6-methyl-5-(1- methylethyl)pyrimidin-4-yl}-9-methyl-2.3.4.5-tetrahydro- 1,4-benzoxazepin-7-yl}pyridin-3- yl)methanesulfonamide. ¹H NMR (400 MHz, CD₃OD) δ 8.48 (d, 1H), 8.17 (d, 1H), 7.50 (s. 1H), 7.50 (s. 1H), 6.29 (t, 1H), 5.04 (s, 2H), 4.57 (t, 2H), 4.50 (s, 2H), 4.19 (t. 21H), 3.64 (dd, 2H), 3.19 (m, 1H), 3.13 (s, 3H), 2.66 (s, 3H), 2.30 (s, 3H), 1.43 (d, 6H), MS (ES) for C₂₇H₃₃ClF₂N₆O₂S: 596 (MH⁺).

2,2-difluoro-N-({4-[7-(1H-imidazo]4,5-b]pyridin-6-yl)-9-methyl-2,3-dihydro-1,4- benzoxazepin-4(5H)-yl]-6-methyl-5-(1-methylethyl)pyrimidin-2-yl}methyl)ethanamine. ¹H NMR (400 MHz, CD₃OD) δ 9.44 (s, 1H), 8.99 (d, 1h), 8.59 (d, 1H), 7.68 (s, 1H), 7.60 (s, 1H), 6.26 (t, 1H), 5.09 (s, 2H), 4.59 (t, 2H), 4.48 (s, 2H), 4.18 (t, 2H), 3.61 (dd,2H), 3.18 (m, 1H), 2.65 (s, 3H), 2.33 (s, 3H), 1.44 (d, 6H), MS (ES) for C₂₇H₃₁F₂N₇O: 508 (MH⁺).

2,2-difluoro-N-([4-methyl-5-(1-methylethyl)-6-[9-methyl-7-(2-methyl-1H- imidazo[4,5-]pyridin-6-yl)-2.3-dihydro-1,4-benzoxazepin-4(5H)-yl]pyrimidin-2- yl]methyl)ethanamine. ¹H NMR (400 MHz, CD₃OD) δ 8.88 (d, 1H), 8.44 (d, 1H), 7.65 (d, 1 H), 7.58 (d, 1H), 6.26(t, 1H), 5.08 (s, 2H), 4.59 (t, 2H), 4.49 (s, 2H), 4.18 (t, 2H), 3.62 (td, 2H), 3,18 (m, 1H), 2.91 (s, 3H), 2.65 (s, 3H), 2.32 (s, 3H), 1.41 (d, 6H), MS (ES) for C₂₈H₃₃F₂N₇O: 522 (MH⁺).

2, 2difluoro-N-([4-[7-(1H-imidazo]4,5-b]pyridin-6-yl)-9-methyl-2,3-dihydro-1,4- benzoxazepin-4(5H)-yl ]-6-methyl-5-(1methylethyl)pyrimidin-2-yl]methyl)-N- methylethanamine. ¹H NMR (400 MHz, d₆-DMSO): 8.61 (br s, 1H), 8.47 (s, 1H), 8.17 (br s, 1H), 7.51 (d, 1H), 7.42 (d, 1H), 6.03 (u, 1H), 4.42 (s, 2H), 4.36-4.24 (m, 2H), 3.71-3.66 (m, 2H), 3.66 (s, 2H), 3.38-3.23 (m, 3H), 2.92 (td, 2H), 2.48 (s, 3H), 2.33(s, 3H), 2.27 (s, 3H), 1.32 (d, 6H), MS (EI) for C₂₈H₃₃F₂N₇O: 522 (MH⁺).

N-ethyl-2,2-difluoro-N-({4-[7-(1H-imidazo]4,5-b]pyridin-6-yl)-9-methyl-2,3- dihydro-1,4-benzoxazepin-4(5H)-yl]-6-methyl-5-(1 -methylethyl)pyrimidin-2- yl]methyl)ethanamine. ¹H NMR (400 MHz, CD₃OD) δ 9.48 (s, 1H), 9.01 (d, 1H), 8.60 (d, 1H), 7.71 (d, 1H), 7.56 (t, 1H), 6.08 (t, 1H), 5.17 (s, 2H), 4.60 (t, 2H), 4.24 (s, 2H), 4.18 (t. 2H), 3.31 (dt, 2H), 3.19 (m, 1H), 3.06 (q, 2H), 2.66 (s, 3H), 2.29 (s, 3H), 1.44 (d, 6H), 1.11 (m, 3H), MS (ES) C₂₉H₃₅F₂N₇O: 536 (MH⁺).

6-{4-[2-(1--aminoethyl)-6-mehyl-5-(1-methylethyl)pyrimidin-4-yl]-9-methyl-2,3,4,5- tetrahydro-1,4-benzoxazepin-7-yl}[1,3]thiazolo[5,4-b]pyridin-2-amine. ¹H NMR-(400 MHz, d₆-DMSO) δ 8.36 (d, 1H), 7.88 (s, 2H), 7.80 (d, 1H), 7.47 (s, 1H), 7.46 (s, 1H), 4.48 (s, 2H), 4.31 (t, 2H), 3.75 (m, 1H), 3.72 (t, 2H), 3.26(m, 1H), 2.47 (s, 3H), 2.25 (s, 3H), 1.32 (d, 3H) 1.29 (d,3H), 1.18 (d, 3H), MS (ES) for C₂₆H₃₁N₇OS:490 (MH⁺).

### EXAMPLE 4: 4-[7-{6-Chloro-5]methylsulfonyl)amino]pyridin-3-yl}-9-methyl-2,3- dihydro-1,4-benzoxazepin-4(5H)-yl]-6-methyl-5-(1-methylethyl)pyrimidine-2-carboxide

STEP 1: A mixture of 1,1-dimethylethyl9-methyl-7-(4,4,5,5-tetramethyl-1,3,2- dioxaborolan-2-yl)-2,3-dihydro-1,4-benzoxazepine-4(5H)-carboxylate (1.0 g, 2.5 mmol), N- (5-bromo-2-chloropyridin-3-yl)methanesulfonamide (733 mg, 2.5 mmol), cesium carbonate (2.4 g, 7,5 mmol), and 1,1-Bis(diphenylphosphino)ferrocene-palladium(II)dichloride diclormethane complex (204 mg, 0.25 mmol) in dioxane (2.4 mL) and water (600 uL) was stirred at 110° C. for 2 h and was then cooled to rt. The mixture was then diluted with water and extracted three times with ethyl acetate. The organic extracts were combined dried over sodium-sulfate, filtered, and concentrated in vacuo. The residue was purified by gradient silica gel chromatography (100% hexanes to 50% ethyl acetate in 50% hexanes) to provide 1,1-dimethyllethyl 7-[6-chloro-5[(methylsulfonyl)amino]pyridin-3-yl]-9-methyl-2,3- dihydro-1,4-benzoxazepine-4(5H)-carboxylate (712 mg, 1.52 mmol, 61% yield) as a light brown film. ¹H NMR (400 MHz, CDCI₃) δ 8.39 (d, 1H), 8.16-8.06 (m, 1H), 7.37-7.18 (m, 2H), 6,86 (br s, 1H), 4.56-4.43 (m, 2H), 4.09-4.05 (m, 2H), 3.88-3.80 (m, 2H), 3.09 (s, 3H), 2.33 (d, 3H), 1.44 (d, 1H); MS (EI) for C₂₁H₂₆ClN₃O₅S: 468, 470 (MH⁺, Cl isotopes).

STEP 2: To a solution of 1,1 -dimethylethyl 7-{6-chloro-5- [(methylsulfonyl)amino[pyridin-3-yl]-9-methyl-2,3-dihydro-1,4-benzoxazepine-4(5H)- carboxylate (712 mg, 1.5 mmol ) in methanol (5 mL) was added hydrogen chloride in dioxane (4 M, 3.75 mL, 15 mml), and the resulting solution was heated TO 60° C. for 30 min. After cooling to rt the volatile materials were removed in vacuo to provide N-[2-chloro-5-(9- methyl-2,3,4,5-tetrahydro-1,4-benzoxazepin-7-yl)pyridin-3yl methanesulfonamide hydrochloride salt in quantitative yield. ¹H NMR (400 MHz, DMSO-d₆) δ 9.90 (s, 1H), 9.39 (br s, 2H), 8.57 (d, 1H), 8.05 (d, 1H), 7.70 (d, 2H), 4.5-4.01 (m, 4H), 3.55-3.47 (m, 2H), 3.17 (s, 3H), 2.30 (s, 3H); MS (EI) for C₁₆H₁₈ClN₃O₃S: 370 (MH⁺, Cl isotopes).

STEP 3: A mixture of N-[2-chloro-5-(9-methyl-2,3,4,5-tetrahydro-1,4- benzoxazepin-7-yl)pyridin-3-yl]methanesulfonamide (414 mg, 0.94 mmol), [4-chloro-6- methyl-5-(1-methylethyl)pyrimidin-2-yl]methyl acetate (211 mg, 0.94 mmol), and diisopropylethylamine (491 uL, 2.8 mmol) in NMP (940 uL) was heated to 120° C. for 16 h before cooling to rt. The mixture was then diluted with water and extracted three-times with ethyl acetate. The organic extracts were combined, dried over sodium sulfate, filtered, and concentrated in vacuo. The residue was purified by gradient silica gel chromatography (100% hexanes to 25% hexanes in ethyl acetate) to provide {4-[7-[6-chloro-5- [(methylsulfonyl)amino-]pyridin-3[-9-methyl-2,3-dihydro-1,4benzoxazepin-4(5H)-yl]-6- methyl-5-(1-methylethyl)pyrimidin-2-yl}methyl acetate (298 mg, 0.519 mmol, 55% yield) as a brown oil. ¹H NMR (400 MHz, CDCI₃) δ 8.40 (d, 1H), 8.14 (d, 1H), 7.33 (d, 1H), 7.23 (d, 1H), 5.09 (s, 2H), 4.37 (s, 2H), 4.32-4.26 (m, 2H), 3.80-3.73 (m, 2H), 3.43-3.33 (m, 1H), 3.10 (s, 3H), 2.56 (s, 3H), 2.33, (s, 3H), 2.18 (s, 3H), 1.36 (d, 6H); (EI) for C₂₇H₃₂ClN₅O₅S: 574, 576 (MH⁺, CI isotopes).

STEP 4: To a solution of (4-[7-{6-chloro-5-[(methylsulfonyl)amino]pyridin-3- yl}-9-methyl-2,3-dihydro-1,4-benzoxazepin-4(5H)-yl]-6-methyl-5-(1-methylethyl)pyrimidin- 2-yl}methyl acetate (298mg, 0.519 mmol) in methanol (2 mL) was added aqueous potassium hydroxide (1 M, 1.56 mL, 1.56 mmol). The solution was stirred at rt for 50 min and was then diluted with ethyl acetate. Brine and saturated aqueous ammonium chloride were then added and the phases were partitioned. The aqueous layer was extracted with ethyl acetate. The combined organic extracts were dried over sodium sulfate, filtered, and concentrated in vacuo to provide N-(2-chloro-5-{4-[2-(hydroxymethyl)-6-methyl-5-(1-methylethyl)pyrimidin-4-yl ]- 9-methyl-2,3,4,5-tetrahydro-1,4benzoxazepin-7]pyridin-3-yl)methanesulfonamide (222 mg, 0.447 mmol, 80% yield) as a yellow-orange film. ¹H NMR (400 MHz, CDCI₃) δ 8.41 (d, 1H), 8.15 (d, 1H), 7.32 (d, 1H), 7.29 (d, 1H), 4.56( s, 2H), 4.47 (s, 2H), 4.32-4.25 (m, 2H), 3.84-3.77 (m, 2H), 3.43-3.29 (m, 1H), 3.11 (s, 3H), 2.56 (s, 3H), 2.32 (d, 3H), 1.38 (d, 6H), 1.29-1.20 (m, 7H); (EI) for C₂₅H₃₀ClN₅O₄S: 532, 534 (MH⁺, CI isotopes).

STEP 5: A solution of N-(2-chloro-5-{4-[2-(hydroxymethyl)-6-methyl-5-(1- methylethyl)pyrimidin-4-yl]-9-methyl-2,3;4,5-tetrahydro-1,4-benzoxazepin-7-yl }pyridin-3- yl)methanesulfonamide(210 mg, 0.39 mmol) in dichloromethane (2 mL) was treated with 1,1,1-tri(acetyloxy)-1,1-dihydro-l2-benziodoxol-3-(1H),-one (251 mg, 0.59 mmol) for 30 min at rt. The mixture was diluted with dichloromcthane and washed with aqueous sodium bisulfate. The aqueous phase was extracted with dichlqronicthane. The combined organic extracts were dried over sodium sulfate, filtered, and concentrated in vacuo. The residue was dissolved in tert-butanol (1.5 mL) and acetonitrile (500 uL). To this solution were added 2- methyl-2-butene (approximately 1 mL) and a solution of sodium chlorite (176 mg, 1.95 mmol) and potassium dihydrogen phosphate(212 mg, 1.56 mmol) in water (1.5 mL). The mixture was stirred for 1 h at rt and then water and ethyl acetate were added. An insoluble white precipitate was removed by filtration and was discarded. The filtrate was partitioned, and the aqueous phase was extracted with ethyl acetate. The combined organic extracts were dried over sodium sulfate, filtered, and concentrated in vacuo. The residue was purified by gradient silica gel chromatography (100% dichloromethane to 20% methanol in dichloromethane) to provide 4-[7-{6-chloro-5-[(methylsulfonyl)amino]pyridin-3-yl)-9- methyl-2,3-dihydro-1,4benzoxazepin-4(5H)-yl]-6-methyl-5-(1-methylethyl)pyrimidine-2- carboxylic acid (136 mg, 0.25 mmol, 64% yield) as a yellow solid. ¹H NMR (400 MHz, DMSO-d₆) δ 8.13 (br s, 1H), 7.87 (s, 1H), 7.46-7.35 (m, 2H), 4.44 (s, 2H), 4.34-4.23 (m, 2H). 3.80-3.70 (m, 2H), 3.33-3.23 (m, 1H), 2.92 (s, 3H), 2.53 (s, 3H), 2.26 (s, 3H), 1.34 (d, 6H): (EI) for C₂₅H₂₈ClN₅O₅S; 546, 548 (MH⁺, CI isotopes).

STEP 6: To a solution of 4-[7-{6-chloro-5-[(methylsulfonyl)amino]pyridin-3yl}- 9 -methyl-2,3-dihydro-1,4-benzoxazepin-4(5H)-yl]-6-methyyl-5-(1-methylethyl)pyrimidine-2- carboxylic acid (73 mg, 0.13 mmol) in DMF (400 uL) was added O-(7-azabenzotriazol-1- yl)N,N,N′,N′-tetramethyluronium hexafluorophosphate (103 mg, 0.27 mmmol) and aqueous ammonia (28-30%, 35 uL, 0.67 mmol). After stirring 40 min. additional O-(7- azabenzotriazol-1-ylN,N,N′,N′-tetramethyluronium hexafluorophosphate (103 mg, 0.27 mmol) and aqueous ammonia (28-30%, 35 uL, 0.67 mmol) were added. The reaction mixture was stirred a further 1.5 h at rt and was then diluted with aqueous lithium chloride (10%). The aqueous solution was extracted twice with ethyl acetate. The combine organic extracts were dried over sodium sulfate, filtered, and concentrated, in vacuo. The residue was purified by preparative reverse phase HPLC to provide 4-[7-{6-chloro-5- [(methylsulfonyl)amino]pyridin-3-yl )-9-methyl-2,3,-dihydro-1,4-benzoxazepin-4(5H)-yl]-6- methyl-5-(1-methylethyl)pyrimidine-2-carboxamide (25.3 mg, 0.046 mmol, 35% yield) as a pale yellow solid. ¹H NMR (400 MHz, DMSO-₆) ε 9.82 (s, 1H), 8.55 (s, 1H), 8.03 (d, 1H), 7.75 (d, 1H), 7.54 (s, 2H), 7.50 (d, 1H), 4.55 (s, 2H), 4.36-4.8 (m, 2H), 3.83-3.75 (m, 2H), 3.31-3.21 (m, 1H), 3.17 (s, 3H), 2.53 (s, 3H), 2.25(s, 3H), 1.33 (d, 6H): MS (EI) for C₂₅H₂₉ClN₆O₄S: 545, 547 (MH⁺, Cl isotopes).

Proceeding according to the method of Example 4 and replacing ammonia in step 6 with N,N-dimethylethylenediamine the following compound of the invention was prepared:

4-[7-{6-Chloro-5-[(methylsulfonyl)amino]pyridin-3-yl}-9-methyl-2,3-dihydro-1,4- benzoxazepin-4(5H)-yl[-N-[2-(dimethylamino)ethyl]-6-methyl-5-(1-methylethyl)pyrimidine- 2-carboxamide. ¹H NMR (400 MHz, DMSO-d₆) δ 8.39 (t, 1H), 8.29 (s, 1H), 7.94 (d, 1H), 7.51-7.42 (m, 2H), 4.55 (s, 2H), 4.36-4.29 (m, 2H), 3.85-3.74 (m, 2H), 3.37-3.22 (m, 3H), 2.99 (s, 3H), 2.61-2.53 (m, 5H), 2.34 (s, 6H), 2.25 (s, 3H), 1.34 (d, 6H); MS (EI) for C₂₉H₃₈ClN₇O₄S: 616, 618 (MH⁺,CI isotopes).

Proceeding according to the method of Example 4 and replacing [4-chlqro-6- methyl-5-(1-methylethyl)pyrimidin-2-yl]methyl acetate in step 3 with alternative reagents and omission of steps 4-6, the following compounds of the invention were prepared:

N-(5-{4-[2-Amino)-6-methyl-5-(1-methylethyl)pyrimidin-4-yl]-9-methyl-2,3,4,5- tetrahydro-1,4-benzoxazepin-7-yl}-2-chloropyidin-3yl)methanesulfonamide. ¹H NMR (400 MHz, DMSO-d₆) δ 9.94 (brs, 1H), 8.49 (s, 1H), 8.00 (d, 1H), 7.52 (d, 1H), 7.39 (d, 1H), 6.08 (br s, 2H), 4.32-4.19 (m, 4H), 3.61-3.49 (m, 2H), 3.23-3.07 (m, 4H), 2.30 (s, 3H), 228 (s, 3H), 1.25 (d, 6H): MS (EI) C₂₄H₂₉ClN₆O₃S: 519 (MH⁺, Cl isotopes).

N-(5-{4[2-Amino-5-(trifluoromethyl)pyrimidin-4-yl]-9-methyl-2,3,4,5-tetrahydro- 1,4-benzoxazepin-7-yl}-2-chloropyridin-3-yl)methanesulfonamide. ¹H NMR (400 MHz,. DMSO-d₆): 9.81 (s, 1H), 8.19 (s, 2H), 8.00 (s, 1H), 7.57 (s, 1H), 7.43 (s, 1H), 7.11-6.62 (m, 2H), 4.83 (s, 2H), 4.29 (s, 2H), 3.89 (s, 2H), 3.13 (s, 3H), 2.22 (s, 3H); MS.(EI) for C₂₁H₂₀Clf₃N₆O₃S: 529 (MH⁺).

N-(5-{4-[4-Amino-5-(trifluoromethyl)pyrimidin-2-yl]-9-methyl-2,3,4,5-tetrahydro- 1,4-benzoxazepin-7-yl}-2-chloropyridin-3-yl)methanesulfonamide. ¹H NMR (400 MHz, DMSO-d₆): 9.81 (s, 1H); 8.44 (s, 1H), 8.08 (s, 1H), 7.96 (s, 1H), 7.67 (s, 1H), 7.40 (s, 1H), 7.18-6.71 (m, 2H), 4.80 (s, 2H), 4.12 (d, 4H), 3.07 (s, 3H), 2.23 (s, 3H): MS (EI) for C₂₁H₂₀ClF_{3l N}₆O₃S: 529 (MH⁺).

2-[7-[6-Chloro-5-](methylsulfonyl)amino]pyridin-3-yl}-9-methyl-2,3-dihydro-1,4- benzoxazepin-4(5H)-yl]-N-methyl-4-(1-methylethyl)-1,3-thiazole-5-carboxamide. ¹H NMR (400 MHz, DMSO-d₆) δ 8.35 (s, 1H), 7.95 (t, 1H), 7.47 (dd, 4H), 4.73 (s, 2H), 4.19 (d, 2H), 4.01 (s, 2H), 3.62 (m, 1H), 3.05 (s, 3H), 2.62 (d, 3H), 2.24 (s, 3H), 1.08 (d, 6H); MS (EI) for C₂₄H₂₈ClN₅O₄S_{2:} 550.1 (MH⁺).

2-[7-{6-Chloro-5-[(methylsulfonyl)amino]pyridin-3-yl}-9-methyl-2,3-dihydro-1,4- benzoxazepin-4(5H)-yl]-N-ethyl-4-(1-methylethyl)-1,3-thiazole-5-carboxamide. ¹H NMR (400 MHz, DMSO-d₆) δ 9.84 (s, 1H), 8.48 (d, 1H), 7.97 (d, 1H), 7.49 (t, 3H), 4.68 (d, 2H). 4.20 (s, 2H), 3.95 (d, 2H), 3.59 (m, 1H), 3.12 (m, 5H), 2.25 (s, 3H), 1.09 (d, 6H), 1.01 (t, 3H); MS (EI) for C₂₅H₃₀ClN₅O₄S: 564.1 (MH⁺).

N-{5-[4-(4-Amino-5-cyanopyrimidin-2-yl)-9-methyl-2,3,4,5-1,4- benzoxazepin-7-yl]-2-chloropyridin-3-yl }methanesulfonamide. ¹H NMR (400 MHz, DMSO- d₆): 9.82 (s, 1H), 8.51 (d, 1H), 8.25 (d, 1H), 8.00 (d, 1H), 7.63 (s, 1H), 7.37 (d, 3H), 4.87 (d, 2H), 4.13 (d, 4H), 3.15 (d, 3H), 2.23 (s, 3H); MS (EI)for C₂₁H₂₀ClN₇O₃S: 486.1 (MH⁺).

Proceeding according to the method of example 4 and replacing N-(5-bromo-2- chloropyridin-3-yl)methanesulfonamide in step 1 with alternative reagents and ]4-chloro-6- methyl-5-(1-methylethyl)pyrimidin-2-yl]methyl acetate in step 3 with alternative reagents and omission of steps 4-6, the following compounds of the invention were prepared:

2-Amino-5-{4-[2-amino-6-methyl-5-(1-methylethyl)pyrimidin-4-yl]-9-methyl- 2,3,4,5-tetrahydro-1,4-benzoxazepin-7-yl]pyridine-3-sulfonamide. ¹H NMR (400 MHz, DMSO-d₆) δ 8.44 (d, 1H), 8.07 (d, 1H), 7.48 (br s, 2H), 7.36 (d, 1H), 7.24 (d, 1H), 6.61 (br s, 2H), 6.01 (s, 2H), 4.26-4.15 (m, 4H), 3.57-3.47 (m, 2H), 3.25-3.14 (m, 1H), 2.30 (s, 3H), 2.26 (s, 3H), 1.25 (d, 6H): MS (EI) for G₂₃H₂₉N₇O₃S: 484 (MH⁺).

N-(5-{4-[2-Amino-6-methyl-5-(1-methylethyl)pyrimidin-4-yl]-9-methyl-2,3,4,5- tetrahydro-1,4-benzoxazepin-7-yl ]pyridin-3-yl)methanesulfonamide. ¹H NMR (400 MHz, DMSO-d₆) δ 8.57 (s, 1H), 8,37 (s, 1H), 7,77 (s, 1H), 7.47 (s, 1H), 7.34 (s, 1H), 5.96 (d, 1H), 4.24 (s, 4H), 3.52 (d, 2H), 3.10 (s, 3H), 2.29 (d, 6H), 1.26 (d, 6H): MS (EI) for C₂₄H₃₀N₆O₃S: 483.2 (MH⁺).

N-(5-{4-[2-Amino-6-methyl-5-(1-methylethyl)pyrimidin-4-yl]-9-methyl-2,3,4,5- tetrahydro-1,4-benzoxazepin-7-yl,)-2-hydroxypyridin-3-yl)methanesulfonamide. ¹H NMR (400 MHz, DMSO-d₆) δ 12.2 (s, 1H), 8.79 (d, 1H), 7.64 (t, 1H), 7.43 (d, 1H), 7.30 (d, 1H), 7.17 (d, 1H), 6.20 (bs, 2H), 4.24 (d, 4H), 3.57 (s, 2H), 3.16 (m, 1H), 3.11 (s, 3H), 2.31 (s, 3H), 2.24 (s,3H), 1.26 (d, 6H); MS (EI) for C₂₄H₃₀N₆O₄S: 499.2 (MH⁺).

N-[5-[4-[2-Amino-6-methyl-5-(1-methylethyl)pyrimidin-4-yl]-9-methyl-2,3,4,5- tetrahydro-1,4-benzoxazepin-7-yl]-2-(methyloxy)pyridin-3-yl]methanesulfonamide. ¹H NMR (400 MHz, DMSO-d₆)- δ 9.33 (d, 1H), 8.27 (t, 1H), 7.82 (t, 1H), 7.40 (m, 3H), 4.67 (s, 2H), 4.36 (d, 2H), 3.96 (s, 3H), 3.86 (s, 2H), 3.07 (s, 3H), 3.00 (m, 1H), 2.37 (s, 3H), 2.24 (s, 3H), 1.27 (d, 6H): MS (EI) for C₂₅H₃₂N₆O₄S: 513.2 (MH⁺).

4-{7-[4-(1H-Imidazol-2-yl)phenyl]-9-methyl-2,3-dihydro-1,4-benzoxazepin-4(5H)- yl )-6-methyl-5-(1-methylethyl)pyrimidin-2-amine. 1H NMR (400 MHz, DMSO-d₆) δ 7.97 (d, 2H) 7.67 (d, 2H), 7.49 (s, 1H), 7.36 (s, 1H), 7.13 (bs, 2H), 6.00(s, 2H), 4.22 (s, 4H), 3.51 (s, 2H), 3.20 (m, 1H), 2.26 (t, 6H), 1.23 (d, 6H): MS (EI) for C₂₇H₃₀N₆): 455.2 (MH⁺).

N-(5(4-[2-Amino-6-methyl-5-(1-methylethyl)pyrimidin-4-yl]-9-methyl-2,3,4,5- tetradhydro-1,4-benzoxazepin-7-yl}-2-chloropyridin-3-yl)-1,1,1-trifluoromethanesulfonamide. ¹H NMR (400 MHz, DMSO-d₆) δ 8.01 (t, 1H), 7.81 (t, 1H), 7.33 (d, 2H), 7.14 (bs, 1H),4.63 (s, 2H), 4.35 (s, 2H), 3.80 (d, 2H), 2.99 (dt, 1H), 2.37 (d, 3H), 2.22 (s,3H), 1.26 (d, 6H); MS (EI) for C₂₄H₂₆CIF₃N₆O₃S: 571.1 (MH⁺).

4-(6-Iodoquinazolin-4-yl)-9-methyl-7-(2-methyl-1H-benzimidazol-6-yl)-2,3,4,5- tetrahydro-1,4,benzoxazepine. ¹H NMR (400 MHz, methanol-d₆): 8.57 (s, 1H), 8,44 (d, 1H), 8.04 (dd, 1H), 7.8 (s, 1H), 7.57-7.45 (m, 5H), 5.00 (s, 2H), 4.51 (m, 2H), 4.23 (m, 2H), 2.58 (s, 3H), 2.34 (s, 3H), 1.96 (s, 3H); MS (EI) for C₂₆H₂₂IN₅O: 547 (MH⁺).

4-[7-(2-amino[1,3]thiazolo[5,4-b]pyridin-6-yl)-9-methyl-2,3-dihydro-1,4- benzoxazepin-4(5H)-yl]-N-ethyl-6-methyl-5-(1-methylethyl)pyrimidine-2-carboxamide. ¹H NMR (400 MHz, d₆-DMSO): 8.40 (d, 1H), 8.25 (t, 1H), 7.87 (s, 2H), 7.84 (d, 1H), 7.57 (d, 1H), 7.50 (d, 1H), 4.59 (s, 2H), 4.35-4.28 (m, 2H), 3.82-3.76 (m, 2H), 3.31-3.23 (m,. 2H). 3.17-3.08 (m, 1H), 2.53 (s, 3H), 2.24 (s, 3H), 1.33,(d, 6H), 0.93 (t, 3H), MS (EI) for C₂₇H₃₁N₇O₂S: 518 (MH⁺).

N-(2-chloro-5-(4-[2-{ (2-fluoroethyl)amino]methyl}-6-methyl-5-(1- methylethyl)pynmidin-4-yl[-9-methyl-2,3,4,5-tetrahydro-1,4-benzoxazepin-7-yl}pyridin-3- yl)methanesulfonamide. ¹H NMR (400 MHz, CD₃OD) δ 8.39 (d, 1H), 8.13 (d, 1H), 7.41 (s, 1H), 7.41 (s, 1H), 4.60 (s, 2H), 4.50 (dt, 2H), 4.35(m, 2H), 3.85 (m, 3H), 3.84 (s, 3H), 3.35 (m, 1H), 3.08 (s, 3H), 2.94 (m, 2H), 2.54 (s, 3H), 2.29 (s, 3H), 1.39 (d, 6H), MS (ES) for C₂₇H₃₄CIFN₆O₃S: 577 (MH⁺).

N-(2-chloro-5-{4-[2-{l(2.2-difluoroethyl)amino[methyl}-6-methyl-5-(1- methylethyl)pyrimidin-4-yl[-9-methyl-2,3,4,5-tetrahydro-1,4-benzoxazepin-7-yl}pyridin-3- yl)methanesulfonadmide. ¹H NMR (400 MHz, CD₃OD) δ 8.48 (d, 1H), 8.17 (d, 1H), 7.50 (s, 1H), 7.50 (s, 1H), 6.29 (t, 1H), 5.04 (s, 2H), 4.57 (t, 2H), 4.50 (s, 2H), 4.19 (t, 2H), 3.64 (dd, 2H), 3.19 (m, 1H), 3.13 (s, 3H), 2.66 (s, 3H), 2.30 (s, 3H), 1.43 (d, 6H), MS (ES) for C₂₇H₃₃ClF₂N₆O₃S: 596 (MH⁺).

### EXAMPLES 5

### 6-{9-Methyl-4-[6-methyl-5-(1-methylethyl)-2-(methylsulfonyl)pyrimidin- 4-yl[-2,3,4,5-tetrahydro-1,4-benzoxazepin-7-yl][1,3thiazolo[5,4pyridin-2-amine

STEP 1: Sodium metal (640 mg, 27.8 mmol) was added to ethanol (40 mL) and was stirred at it until it completely reacted to form a sodium ethoxide solution. Ethyl 2- acetyl-3-methylbutanoate (2.08 mL, 1.6 mmol) and thiourea (1.06 g, 14 mmol) were then added. The resulting reaction mixture was heated to 80° C. and stirred for 6 h before being cooled to rt. The volatile materials were removed in vacuo. The residue was dissolved in water which was subsequently acidified by addition of acetic acid. The while precipitate that formed was isolated by filtration and then dried in vacuo to provide 6-methyl-5-(1- methylethyl)-2-thioxo-2,3-dihydropyrimidin-4(1H)-one(1.19 g, 6.46 mmol, 56% yield) as a white crystalline solid. ¹H NMR (400 MHz, DMSO-d₆) δ 12.29-11.88 (m, 2H), 2.89-2.77 (m, 1H), 2,13 (s, 3H), 1.16 (d, 6H); (EI) for C₈H₁₂N₂OS: 185 (MH⁺).

STEP 2: To a solution of 6-methyl-5-(1-methylethyl)-2-thioxo-2,3- dihydropyrimidin-4(1H)-one (1.19 g, 6.46 mmol) in DMF (7 mL) was added iodomethane (811 uL, 13 mmol), and the resulting mixture was stirred at rt for 90 min. The mixture was then diluted with 10% aqueous lithium chloride and extracted twice with ethyl acetate. The organic extracts were combined, washed once with 10% aqueous lithium chloride, dried over sodium sulfate, filtered, and conceniraicsd in vacuo to provide 6-methyl-5-(1-methylethyl)-2- (methylthio)pyrimidin-4(3H)-one (1.11 g, 5.6 mmol, 86% yield) as a pale yellow solid. ¹H NMR (400 MHz, CDCI₃) δ 11.73 (s, 1H) 3.09-2.96 (m, 1h), 2.56 (s, 3H), 2.32 (s, 3H). 1.31 (d, 6H); (EI) for C₉H₁₄N₂OS: 199 (MH⁺).

STEP 3: To 6-methyl-5-(1-methylethyl)-2-(methylthio)pyrimidin-4(3H)-one (1.11 g 5.6 mmol) was added chldroform (8 mL) and phosphorus oxychloride (8 mL), and the resulling mixture was heated to 70° C. for .45 min. After cooling to rt, the mixture was concentrated in vacuo. The residue was diluted with dichloromethane and then washed with saturated aqueous sodium bicarbonate. The aqueous wash was extracted with dichloromcthane. The organic extracts were combined, dried over sodium sulfate, filtered, and concentrated in vacuo to provide 4-chloro-6-meihyl-5-(1-methylethyl)-2- (methylthio)pyrimidine (1.20g, 5.55 mmol, 99% yield) as a yellow oil. ¹H NMR (400 MHz, CDCI₃) δ 3.51-3.39 (m, 1H), 2.57-2.51 (m, 6H), 1.36 (d, 6H), (EI) for C₉H₁₃CIN₂OS: 217, 219(MH⁺, CI isotopes).

STEP 4: A mixiture of 6-(9-methyl- 2,3,4,5tetrahydro-1,4-benzoxazepin-7- yl)[1,3]thiazolo[5,4-b]pyridin-2-amine hydrochloride (400 mg, 1.04 mmol), 4-chloro-6- metcihyl-5-(1 -methylethyl)-2-(methylthio)pyrimidine (226 mg, 1.04 mmol), and diisopropylethylamine (724 uL. 4.16 mmol) in NMP (2 mL) was heated to 120° C. for 18 h then cooled to rt. The mixture was then diluted with water and extracted several times, with 10% methanol in ethyl acetate. The organic extracts were combined, dried over sodium sulfate, filtered, and concentrated in vacuo. The residue was purifiedby gradient silica gel chromatography (100% dichloromethane to 10% methanol in dichloromethane) to provide 70% pure 6-(9-methyl-4-[6-methyl-5-(1-methylethyl)-2-(methylthio)pyrimidin-4-yl]-2,3,4,5- tetrahydro-1,4-benzoxazepin-7-yl [1.3]thiazolo[5,4-b]pyridin-2-amine (235 mg, 0.476 mmol, 46% yield) as a viscous brown syrup. ¹H NMR (400 MHz, CDCI₃) δ 8.42 (d, 1H), 7.83 (d, 1H), 7.34 (d, 1H), 7,26.(t, 1H), 5,66 (br s. 1H), 4.4 (s, 2H), 4.35-4.27 (m, 2H), 3.85-3.74 (m, 2H), 3.37-3.26 (m, 1H), 2.50 (s, 3H), 2.44 (s, 3H), 2.34 (s, 3H), 1.36 (d, 6H), (EI) for C₂₅H₂₈N₆O₅S₂: 493 (MH³⁰).

STEP 5: To a solutioin of 6-{9-methyl-4-[6-methyl-5-(1-methylethyl)-2- (methylthio)pyrimidin-4-yl]-2,3,4,5-tetrahydro-1,4-benzoxazepin-7-yl][1,3]thiazolo[5,4- b]pyridin-2-amine(235 mg, 0.476 uiniol) in dichloromethane (5 mL) Was added 3- chloroperbenzoic acid (207 mg, 1.2 mmol). The mixture was stirred for 1 h at rt and then was diluted with dichloromethane. The organic mixture was washed, with aqueous saturated sodium bicarbonate. The aqueous wash was extracted with dichloromethane. The combined organic extracts were dried over sodium sulfate, filtered, and concentrated in-vacuo. A portion of the residue was purified by gradient silica gel chromatography (100% hexanes to 100% ethyl acetate) to provide 6-[9-methyl-4--[6-methyl-5-(1-methylethyl)-2- (methylsulfonyl)pyrimidin-4-yl]-2,3,4,5-tetrahydro-1,4-benzoxazepin-7-yl][1,3]thiazolo[5,4- b]pyridin-2-amine as a yellow solid. ¹H NMR (400 MHz, DMSO-d₆) δ 8,38 (d, 1H), 7,86 (s, 2H), 7.83 (d, 1H), 7.51 (s, 1H), 7.48 (s, 1H), 4.68 (s, 2H), 4.41-4.34 (m, 2H), 3.87-3.79 (m, 2H), 3.29-3.18 (m, 1H), 3.07 (s, 3H), 2.56 (s, 3H), 2.23 (s, 3H), 1.35 (d, 6H): MS (EI) for C₂₅H₂₈N₆O₃S₂: 525 (MH⁺),

Proceeding accordingito the method of example 5, the following compound of the invention was prepared:

6- [9-methyl-4-]6-methyl-5-(1 -methylethyl)-2-(methylsulfinyl)pyrimidin-4-yl ]- 2,3,4,5-tetrahydro-1,4-benzoxazepin-7-yl ][1,3thiazolo[5,4-b]pyridin-2-amine. ¹H NMR (400 MHz, methanol-d₄): 8.37 (d, 1H), 7.84 (d, 1H), 7.45 (d, 1H), 7.39 (d, 1H), 4.70 (m, 2H). 4.37 (m, 2H), 3.91 (m, 2H), 2.66 (s, 3H), 2.60 (s, 3H), 2.28 (s, 3H), 1.41 (m, 6H), MS (EI) for C₂₅H₂₈N₆O₂S₂: 509 (MH⁺).

### EXAMPLE 6

### N′-[4-[7-(2-Amino[1,3thiazolo[5,4-]pyridin-6yl)-9-methyl-2,3-dihydro- 1,4-benzoxazepin-4(5H)-yl]-6-methyl-5-(1-methylethyl)pyrimidin-2yl)-N,N-dimethylethane-1,2-diamine

STEP 1: A solution of 6-[9-methyl-4-[6-methyl-5-(1-methylethyl)-2- (methylsulfonyl)pyrimidin-4-yl]-2,3,4,5-tetrahydro-li4-benzoxazepin-7-yl][91,3thiazolo[5,4- b]pyridin-2-amine (100 mg, 0.19 mmol) in N,N-dimethylethylenediamine (1 mL) was heated to 100° C. for 2 h. After cooling to rt. the volatile materials were removed in vacuo. The reisdue was purified by preparative reverse phase HPLC to provide N′-[4--[7-(2- amino[81,3]thiazolo[5,4-b]pyridin-6-yl)-9-methyl-2.3-dihydro-1,4-benzoxazepin-4(5H)-yl]-6- methyl-5-(1-methylethyl)pyrimidin-2-yl}-N,N-dimethylethane-1,2-diamime (35.0 mg. 0.066 mmol. 35% yield) as a white powder. ¹H NMR (400 MHz. DMSO-d₆) δ 8.34 (d. 1H), 7.87 (s, 2H), 7.78 (d, 1H), 7.48 (s, 1H), 7.39 (s. 1H), 6.24-6.15 (m, 1H). 4,33 (br s. 2H), 4.29-4.23 (m, 2H), 3.64-3.55 (m, 2H), 3.24-3.11 (m, 3H), 2.29 (s, 3H), 2.27-2.22 (m, 5H), 2.09 (s, 6H), 1.26 (d, 6H); MS (EI) for C₂₈H₃₆N₈OS: 533 (MH⁺).

Proceeding according io the method of Example 6 and replacing N,N- dimethylethylenediamine with mclhyiamine the following compound of the invention was prepared:

6-[9-methyl-4-[6-methyl-2-(methylamino)-5-(1-methylethyl)pyrimidin-4-yl]-2,3,4,5- tetrahydro-1,4-benzoxazepin-7-yl)[1,3]thiazolo[5,4-b]pyridin-2-amine. ¹H NMR (400 MHz, DMSO-d₆)-δ 8.34 (d, 1H), 7.87 (s, 2H), 7.77 (d, 1H), 7.49 (d, 1H), 7.39 (d, 1H), 6,40 (g, 1H), 4.32 (s, 2H), 4.30-4.19 (m, 2H), 3.68-3.50 (m, 2H), 3.26-3.09 (m, 1H), 2.66 (d, 3H), 2.30 (s, 3H), 2.26 (s, 3H), 1.26 (d, 6H): MS (EI) for C₂₅H₂₉N₇OS: 476 (MH⁺).

6-]9-methyl-4-[6-methyl-5-(1-methylethyl)-2-(4-methylpiperzaing-1-yl)pyrimidin-4- yl]-2,3,4,5-tetrahydro-1,4-benzoxazepin-7-yl ][1,3]thiazolo[5,4-b]pyridin-2-amine. ¹H NMR (400 MHz, methanol-d₄): 8.34 (d, 1H), 7.81 (d, 1H), 7.38 (m, 2H), 4.62 (s, 2H), 4.50 (s, 2H). 4.30 (m, 2H), 3.74 (m, 2H), 3.62 (m, 4H), 2.40 (m, 4H), 2.38 (s, 3H), 2.31 (s, 3H), 2.29 (s, 3H), 1.34 (d, 6H), MS (EI) for C₂₉H₃₆N₆S: 545 (MH⁺).

1-[4-[7-(2-amino[1,3]thiazolo[5,4-b]pyridin-6-yl)-9-methyl-2,3-dihydro-1,4- benzoxazepin-4(5H)-yl]-6-methyl-5-(1-methylethyl)pyrimidin-2-yl)azetidin-3ol. ¹H NMR (400 MHz, CD₃OD) ε 8.35 (d, 1H), 7.82 (d, 1H), 7.44 (s, 1H), 7.38 (s, 1H), 4.91 (s, 2H), 4.83(m, 1 H), 4.50 (m, 3H), 4.24 (m, 2H), 4.01 (m, 2H), 3.80 (m, 2H), 3.12(m, 1H), 2.47 (s, 3H), 2.26 (s, 3H), 1.39 (d, 6H), MS (ES) for C₂₇H₃₁N₇O₂S: 518 (MH⁺).

### EXAMPLE7

### 4-[7--(5-amino)-1,3,4-thiadiazol-2-yl)-9-methyl-2,3-dihydro-1,4- benzoxazepin-4(5H)-yl]-6-methyl-5-(1-methylethyl)pyrimidin-2-amine

STEP 1: A mixture of 1,1-dimethylethyl-7-bromo)-9-methyl-2,3-dihydro-1,4- benzoxazepine-4(5H)-carboxylate, (4.90 g, 14.92 minmol) and zinc cyanide (1.76 g, 14.92 mmol) in N,N-dimethyfornamide (30 mL) was degassed with nitrogen then. terakis(triphenylphosphine)palladium(0) (0.86 g, 0.75 mmol) was added to the mixture and it was heated at 85° C. for 2.5 hours. After cooling to room temperature the reaction mixture was partitioned between water (100 mL) and ethyl acetate (300 mL). The organic layer was separated washed with water (2× 150 mL) and brine, dried over anhydrous magnesium sulfate then, filtered and concentrated. Gradient silica gel column chromatography (hexane:ethyl acetate 99:1 to 9:1) provided 1,1-dimethylethyl-7-cyano-9-methyl-2,3-dihydro-1,4- benzoxazepine-4(5W)-carboxylate (4.02 g. 93%), MS (EI) for C₁₆H₂₀N₂)₃: 232 (M-tBu⁺).

STEP 2: A mixture of 1 ,l-dime.lhylcthylr7-cyanb-9-mcih:yl-2.3-clihydro-1,4- benzoxazcpine-4(5H)-carboxylate (2.3 g, 7.98 mmol) and thiosemicarbazide (0.76 g, 8.38 mmol) in trifluorbacetic acid (20 mL) was heated to reflux for 6 hours. After cooling to room temperature the reaction mixture was concentrated then taken into 1,4-dioxane and concentrated (3× 50 mL) to give crude 5-(9-methyl-2;3,4,5-tetrahydro-1,4-benzoxazepin-7- yl)-1,3,4-thiadiazol-2-amine. MS (EI) for C₁₂H₁₄N₄OS: 263 (MH⁺). To a solution of 5-(9- methyl-2,3,4,5-tetrahydro-1.4-benzoxazepin-7-yl)-1,3,4-thiadiazol-2-amine as obtained above in a mixture of water (70 mL) and tetrahyrofuran (1 mL) wvas added 2M aqueous sodium hydroxide (20 mL, 40 mmol) and the reaction mixture was cooled to 0° C., followed by the addition of di-tert-butyldicarbonate (1.92 g. 8.78 mmol) then stirred at room temperature for 18 hours. Thereaction mixture wasqiartitioheel between water (200 mL) and ethyl acetate (300 mL). The organic layer-was separated washed with water (25 mL) and brine then dried over anhydrous magnesium sulfate, filteredd and concentrated. Gradient column chromatography (hexane:ethyl acetate 9:1 to 3:2) provided 1,1 -dimetliylethyl-7--(5- amino-1,3,4-thiadiazol-2-yl)-9-methyl-2,3-dihydro-1,4-benzoxazepine-4(5H)-carboxylate (1.22 g, 41%). ¹H NMR (400 MHz, DMSO-d₆): 7.42 brs,(2H), 7.33 (s, 2H), 4.42 (brs. 2H). 4.02 (m, 2H), 3.72 (brs, 2H), 2.22(s, 3H), 1.35 (s, 9H): MS(EI) for C₁₇H₂₂N₄O₃S: 363(MH⁺).

STEP 3: A solution of 1.1 -dimethylethyl-7--(5-amino-1.3.4-thiadiazol-2-yl)-9- methyl-2,3-dihydro-1,4-benzoxazepine-4(5H)-carboxylate (1.20 g, 3.31 mmol) in a mixture of methanol (25 mL) and 4N hydrochloric acid in 1.4-dioxane (5 mL) was refluxed for 30minutes. After cooling to room temperature the reaction mixture was concentrated and the precipitate was collected by filtration, washed with ethyl acetate and hexanes then dried in vacuo to give 5-(9-methyl-2,3,4,5-tetrahydro-1,4-beenzoxazepin-7-yl)-1,3,4-thiadiazol-2- amine dihydrochloride salt (1.0 g, 94%) as a white solid. ¹H NMR (400 MHz, DMSO-d₆): 9.71 (s, 2H), 7.78 (s, 1H), 7.68 (s, 1H), 4.38 (brs, 2H), 4.24 (m, 2H), 3.45 (brs, 2H), 2.24 (s. 3H): MS (EI) for C₁₂H₁₄N₄OS: 263 (MH⁺).

STEP 4: A mixture of 5-(9-methyl-2,3,4,5-tetrahydro-1,4-benzoxazepin-7-yl)- 1,3,4-thiadiazol-2-amine dihydrochloride salt (0.22 g, 0.70 mmol), 4-chloro-6-methyl-5-(1- methylethyl)pyrimidin-2-amine (0.12 g, 0.65 mmol) and N,N-diisopropylethylamine (0.60 mL, 3.5 mmol) in 1-methyl-2-pyrrolidinone (2 mL) was heated,at 110° C. for 18 hours. After cooling to room temperature the reaction mixture was diluted with methanol (6 mL) and water (4 mL) and the pH was adjusted lo 5 by the addition of glacial acetic acid then purified by preparative reverse phase HPLC (0.1 % aqueous ammonium acetate and acetonitrile mobile phase). Product fractions were concentrated and the residue was partitioned between 2M aqueous sodium hydroxide (100 mL) and ethyl acetate (250 mL). The organic layer was separated washed with 2M aqueous sodium hydroxide (100 mL) and brine, dried over anhydrous magnesium sulfate then filtered and concentrated. The residue was dissolved in ethanol (20 mL) and concentrated aqueous hydrochloric acid (1.0 mL) was added and the solvent was partially concentrated. The solid precipitate was collected by filtration washed with ethyl, acetate and hexanes then dried in vacuo to give 4-[7-(5-amino- 1,3,4-thiadiazol-2- yl)-9-methyl-2,3-dihydro- 1,4-benzoxazepin-4(5H)-yl]-6-methyl-5-(1-methylethyl)pyrimidin- 2-amine hydrochloride (78 mg, 27%), 1H NMR (400 MHz, methanol-d₄): 7.67 (d, 1H), 7.54(d, 1H), 4:84 (s, 2H), 4.50 (m, 2H), 4.02 (m, 2H), 3.01 (m, 1H), 2.44 (s, 3H) 2.25 (s, 3H). 1.34 (d, 6H), MS (EI) for C₂₀H₂₅N₇OS: 412 (MH⁺).

Proceeding according to the method of example 7 and replacement of 4-chloro-6- melhyl-5-(1-methylethyl)pyrimidin-2-amine with alternative reagents the following compounds of the invention were prepared:

5-[4[(2-(2,2-difluoroethyl)(methyl)amino]methyl]-6-methyl-5-(1- methylethyl)pyrimidin-4-yl ]9-methyl-2,3,4,5-tetrahydro-1,4-benzoxazepin-7-yl]-1,3,4- thiadiazol-2-amine. ¹H- NMR (400 MHz, d₆-DMSO): 7.50 (d, 1H), 7.47 (d, 1H), 7.32 (s, 2H). 6.03 (t, 1H), 4.39 (s, 2H), 4.35-4.28 (m, 2H), 3.72-3.66 (m, 2H), 3.64 (s, 2H), 3.27-3.15 (m, 1H), 2.91 (td, 2-H), 2.47 (s, 3H), 2.32 (s, 3H), 2.22 (s, 3H), 1.3 (d, 6H). MS (EI) for C₂₄H₃₁F₂N₇OS: 504 (MH⁺).

5-(4-[2-[[(2,2-difluoroethyl)amino]methyl]-6-methyl-5(1 -methylethyl)pyrimidin-4- yl -9-methyl-2,3,4,5-tetrahydro-1,4-benzoxazepin-7-yl]-1,3,4-thiadiazol-2-amine. ¹H NMR (400 MHz, CD₃OD), δ 7.78 (s, 1H), 7.61 (s, 1H), 6.34 (l, 1H), 5.01 (s, 2H), 4.60 (d, 2H), 4.44 (s, 2H), 4.14 (t, 2H), 3.62 (m, 2H), 3.14 (m, 1H), 2.64 (s,3H), 2.27 (s, 3H), 1.41 (d, 6H), MS (ES) C₂₃H₂₉F₂N₇OS: 490 (MH⁺).

5-[4-[2-[(2,2-difluoroethyl)(ethyl)aminio]methyl]-6-methyl-5-(1- methylethyl)pyrimidin-4-yl]-9 -mmethyl-2,3,4,5-tetrahydro-1,4-benzoxazepin-7-yl]-1,3,4- thiadiazol-2-amine. ¹H NMR (400 MHz, CD₃OD) δ 7.79 (s, 1H), 7.58 (s, 1H), 6.07 (t, 1H), 5.12 (s, 2H), 4.6 (t, 2H), 4.15 (t, 2H),4.13 (s, 2H), 3.19 (m, 2H), 3.15 (m, 1H), 2.98 (q, 2H), 2.65 (s, 3H), 2.23 (s, 3H), 1.43 (d, 6H), 1.11 (t, 3H), MS (ES) for C₂₅H₃₃F₂N₇OS: 518 (MH⁺).

N-ethyl-2,2-difluoro-N-([4-[7-(1H-inutlazo[4,5-b]pyridin-6-yl)-9-methyl-2.3- dihydro-1,4-benzoxazepin-4(5H)-yl]-6methyl-5-(1-methylethyl)pyrimidin-2- yl]methyl)ethanamine, ¹H NMR (400 MHz, CD₃OD) δ 9.48 (s, 1H); 9.01 (d, 1H), 8.60 (d, 1H), 7.71 (d, 1H), 7.56 (t, 1H), 6.08 (t, 1HHH), 5.17 (s, 2H), 4.60 (t, 2H), 4.24 (s, 2H), 4.18 (t, 2H), 3.31 (dt, 2H), 3.19 (m, 1H), 3.06 (q, 2H), 2.66 (s, 3H), 2.29 (s, 3H), 1.44(d,6H), 1.11 (m, 3H), MS,(ES) for C₂₉H₃₅F₂N₇O: 536 (MH⁺).

5- (4-[2,6-dimethyl-5-( 1-methylethyl)pyrimidin-4-yl ]-9-methyl-2,3,4,5-tetrahydro- 1,4-benzoxazepin-7-yl]-1,3,4-thiadiazol2-amine. 1H NMR (400 MHz, methanol-d₄): 7.53 (m, 2H), 4.47 (s, 2H), 4.32 (m, 2H), 3.81 (m, 2H), 2.50 (s, 3H), 2.40 (s, 3H), 2.27 (s, 3H), 1.36 (d, 6h), MS (EI) for C₂₁ H₂₆N₆OS: 411 (MH⁺).

5-[9-methyl-4-[6-methyl-5-(1-methylethyl)pyrimidin-4yl]-2,3,4,5-tetrahydro-1,4- benzoxazepin-7-yl]-1,3,4-thiadiazol-2-amine. ¹H NMR (400 MHz, methanol-d₄): 8.35 (s, 1H), 7.52 (m, 2H), 4.47 (s, 2H), 4.35 (m, 2H), 3.81 (m, 2H), 2.54 (s, 3H), 2.28 (s, 3H), 1.37 (d, 6H), MS (EI) for C₂₀H₂₄N₆OS: 397 (MH⁺).

5-[4-(2,5-dimethylpyrimidim-4-yl)-9-methyl-2,3,4,5-tetrahydro-1, 4-benzoxazepin-7- yl]-1,3,4-thiadiazol-2-amine. ¹H NMR (400 MHz, methanol-d₄): 7.99 (s, 1H), 7.76 (d, 1H), 7.50 (d, 1H), 5.14 (s, 2H), 4.42 (m, 4H), 2.57 (s, 3H), 2.42 (s, 3H), 2.25 (s, 3H), MS (EI) for C₁₈H₂₀N₆OS: 369 (MH⁺).

5-(9-methyl-4-[2methyl-5-(1-methylethyl)pyrimidin-4yl -]-2,3,4,5-tetrahydro-1.4- benzoxazepin-7-yl]-1,3,4-thiadiazol-2-amine, ¹H NMR (400 MHz, methanol-d₄): 8.16 (s, 1H), 7.55 (d, 1H), 7.50 (d, 1H), 4.64 (s, 2H), 4.34 (m, 2H), 3.94 (m, 2H), 2.41 (m, 1H), 2.41 (s, 3H), 2.26 (s, 3H), 1.26 (d, 6H), MS (EI) for C₂₀H₂₄N₆OS; 397 (MH⁺).

5-[4-(5,6-dimethylpyrimidin-4-yl)-9-methyl-2,3,4,5-tetraydro-1,4-benzoxazepin-7- yl -1,3,4-thiadiazol-2-amine, ¹H NMR (400 MHz, methanol-d₄): 8.29,(s, 1H), 7.52 (d, 1H), 7.46 (d, 1H), 4.65 (s, 2H), 4.36 (m, 2H), 3.92 (m, 2H), 2.39 (s, 3H), 2.26 (s, 3H), 2.24 (s, 3H), MS (EI) for C₁₈H₂₀N₆OS: 369 (MH⁺).

5-[9-methyl-4--[5-(1-methylethyl)pyrimidin-4-yl]-2,3,4,5-tetrahydro-1,4- benzoxazepin-7-yl]-1,3,4-thiadiazol-2-amine. ¹H NMR (400 MHz, methanol-d₄): 8.42 (s, 1H), 8.30 (s, 1H), 7.51 (m, 2H), 4.67 (s, 2H), 4.39 (m, 2H), 3.94 (m, 2H), 3.14 (m, 1H), 2.26 (s, 3H), 1.29 (d, 6H), MS (EI) for C₁₉H₂₂N₆OS: 383 (MH⁺).

4-[7-(5-amino-1,3,4-thiadiazol-2-yl)-9-methyl-2,3-dihydro-1,4-benzoxazepin-4(5H)- yl]-5-methylpyrimidin-2-amine. ¹H NMR (400 MHz, methanol-d₄): 7.66 (d, 1H), 7.56 (s, 1H), 7.42 (d, 1H), 4.83 (s, 2H), 4.32 (m, 2H), 4.13 (m, 2H), 2.26 (s, 3H), 2.21 (s, 3H), 1.95 (s, 3H). MS (EI) for C₁₇H₁₉N₇OS: 370 (MH⁺).

4-[7-(5-amino-1,3,4-thiadiazol-2-yl)-9-methyl-2,3-dihydrol-1,m4-benzoxazepin-4(5H)- yl]-5,6-dimethylpyrimidin-2-amine. ¹H NMR (400 MHz, methanoL-d₄): 7.68 (d, 1H), 7.55 (d, 1H), 4.98 (s, 2H), 4.48 (t, 2H), 4.15 (t, 2H), 2.33 (s, 3H), 2.26 (s, 3H), 2.25 (s, 3H), MS (EI) for C₁₈H₂₁N₇OS: 384 (MH⁺).

4-[7-(5-amino-1,3,4-thidiazol-2-yl)-9-methyl-2,3-dihydro-1,4-benzoxazepin4.(5H)- yl]-5-(1-methylethyl)pyrimidin-2-amine. ¹H NMR (400 MHz, methanol-d₄): 7.80 (s, 1H), 7.57 (d, 1H), 7.46 (d, 1H), 4.66 (s, 2H), 4.36 (m, 2H), 3.93 (m, 2H); 3.05 (m, 1H), 2.26 (s, 3H), 1.95 (s, 3H), 1.22 (d, 6H), MS (EI) for C₁₉H₂₃N₇OS: 398 (MH³⁰).

4-[7-(5-amino-1,3,4-thiadiazol-2-yl)-9-methyl-2,3-dihydro-1,4-benzoxazepin-4(5H)- yl]-5-ethenyl-6-mcthylpyrimidin-2-amine. ¹H-NMR (400 MHz, d₄-MeOH): 7.68 (s, 1H),. 7.57 (s, 1H), 6.55 (dd, 1H), 5.65 (d, 1H), 5.33 (d, 1H), 5.05 (s, 2H), 4.41 (tr, 2H), 4.19 (tr, 2H), 2.32 (s. 3H), 2.25 (s. 3H), MS (EI) for C₁₉H₂₁N₇OS: 396 (MH³⁰ ).

### Biological Example 1

### mTOR/GbL/Raptor (mTQRC1) ELISA Assay

The measurement of mTORC1 enzyme activity was performed in an ELISA assay format following the phosphorylation of 4E-BP1 protein. All experiments were performed in the 384-well format. Generally, 0.5 μL DMSO containing varying concentrations of the test Compound was mixed with 15 μL enzyme solution. Kinase reactions were initiated with the addition of 15 μL of substrates-containing solution. The assay conditions were-as follows; 0.2 nM mTORC1, 40 uM ATPand 50 nM NHis-tagged4E-BP1 in20 mM Hepes, pH 7.2, 1 mM DTT, 50 mMNaC1, 10 mM MnC1₂, 0.02 mg/mL BSA, 0.01% CHAPS, 50 mM β-glycerophosphate. Following an incubation of 120 minutes at anibient temperature, 20 μL of the reaction volume was transferred to a Ni-Chelate-coated 384-well plate. The binding step of the 4E-BP1 protein proceeded for 60 minutes, followed by washing 4 times each with 50 uL of Tris-buffered saline solution (TBS). Anti-phospho-4E-BPl rabbit-IgG (20 pL. 1:5000) in 5% BSA-TBST (0.2% Tween-20 in TBS) was added and further incubated for 60 minutes. Incubation with a secondary HRP-tagged anti-IgG was similarly performed after washing off the primary antibody (4 washes of 50 μL). Following the final wash step with TBST, 20 μL of SuperSignal ELISA Femto (Pierce Biotechnology) was added and the luminescence measured using an En Vision plate reader.

As numbered in Table 1, Compounds 2-3, 5-6, 8-12, 14, 16, 17, 19-23, 28, 30-32, 34, 40-44, 46, 49-59, 61-67, 80, 84, 85-94, 98-99, 104-108, 113, 115-116, 119-130, 132-139, 141-147, 149-156, 158-166, 168-169, 172-181 have an IC₅₀ in this assay of less than or equal to 100 nM.

As numbered in Table 1. Compounds 7, 13, 15, 18, 24-27, 33, 35-39, 45, 47-48, 60, 68-79, 81-83, 100, 103, 109-112, 114, 117-118, 131, 140, 148, 157, 167, 170-171 have an IC₅₀ in this assay of greater than 100 nM but less than or equal to 500 nM.

### Biological Example 2

### Immune-Complex mTORC2 Kinase (mTORC2 IP-Kinase) Assay

HeLa (ATCC) cells are grown in suspension culture and lysed in ice-cold lysis buffer containing 40 mM HEPES pH 7.5, 120mM NaCl, 1 mM EDTA, 10 mM sodium pyrophosphate, 10 mM β-giyccrophosphatc, 10 mM NaF, 10 mM NaN₃, one tablet of protease inhibotors (Complete-Mini, EDTAfrecc, Roche), 0.3% cholamidopropyldimethylaminoniopropanesulfonate (CHAPS), 1 mM AEBSF, 0.5 mM benzamidine HCl, 20 μg/mL heparin, and 1.5 mM Na₃VO₄. The MTORC₂ complex is immunoprecipitatcd with anti-RICTOR antibody for 2 h. The immune complexes are immobilized on Protein A sepharose (GE Healthcare, 17-5280-01), washed sequentially 3 times with wash buffer (40 mM HEPES pH 7.5, 120 mM NaCI, 10 mM β-glycerophosphate, 0.3% CHAPS, 1 mM AEBSF, 20 μg/mL heparin, 1.5 mM Na₃VO₄, and Complete-Mini, EDTA-free) and resuspended in kinase buffer (40 niM HEPES, pH 7.5, 20 mM NaCI, 0.3% CHARS, 20 μg/mLheparin, 4 mM Mgcl₂, 4 mM MnCl₂, 10% Glycerol, and 10 mM DTT); The immune complexes (equivalent to 1×10⁷ cells) are pre-incubatcd at 37° C. with a test Compound or 0.6% DMSO for 5 min, and then subjected to a kinase reaction for 8 min in a final volume of 33 μL (including 5 μL bed volume) containing kinase buffer. 50 μM ATP, and 0.75 μg full length dephosphorylated AKT1, Kinase reactions are tenninated by addition of 11 μL 4×SDS sample buffer containing 20% β-mercaptoethanol and resolved in a 10% Tris Glycine gels. The gels are transferred onto PVDF membrane at 50 V for 20 h at 4 ° C. The membranes are blocked in 5% non-fat milk in TBST for 1 h and incubated overnight at 4° C. with 1/1000 dilution of rabbit anti-pAKT(S473) (Cell Signaling Technology, 4060) in 3% BSA/ TBST. The membranes arcwashed 3 times in TBST and incubated for 1 h with a 1/10000 dilution of secondary goat anti-rabbit HRP antibody (Cell Signaling Technology, 2125) in 5% non-fatmilk/TBST. The signal is detected using Amcrsham ECL-plus. The scanned data are analyzed using ImageQuanl software. IC₅₀ for the test Compound is determined relative to DMSO treated sample using XLfil4 software.

### Biological Example 3

### PI3K Biochemical Assays

P13Kα activity was measured as the percent of ATP consumed following the kinase reaction using luciferase-luciferin-coupled chemiluminescence. Reactions were conducted in 384-well white, medium binding microliter plates (Greiner). Kinase reactions were initiated by combining test compounds. ATP, substrate (PIP2), and kinase in a 20 μL volume in a buffer solution. The standard P13Kalpha assay buffer was composed 50 mM Tris, pH 7.5, 1 mM EGTA, 10 mM MgCl₂, 1 mM DTT and 0.03% CHAPS. The standard assay concentrations for enzyme, ATP, and substrate were 3 nM, IpM, and 10 μM, respectively. The reaction mixture was incubated at ambient temperature for approximately 2 h. Following the kinase reaction, a 10 μL aliquot of luciferase-luciferin mix (Promega Kinase-Glo) was added and the chemiluminescence signal measured using a Victor2 or EnVision (Perkin Elmer). Total ATP consumption was limited to 40-60% and IC50 values of control compounds correlate well with literature references. Substituting P13Kα with P13Kβ, P13Kε, or PI3Kδ, the inhibitory activity of the compounds for the other isoforms of P13-K were measured. For the P13Kβ and P13Kβ assays, enzyme concentrations were 10 nM and 4 nM. respectively. For the P13γ assay, enzyme concentration was 40 nM, the incubation time was 1 h, and the concentration of MgCl₂ in the assay buffer was 5 mM.

As numbered in Table I, Compounds 2, 3, 5-12, 14-16, 18, 20, 21, 23, 26, 28, 30- 32, 35, 40, 41, 43-59, 61-67, 70, 72-76, 78-94, 98-105, 107-109, 113, 115-116, 119-130, 132- 147, 149-173, 175-182 have an IC₅₀ in the P13K-alpha assay of less than or equal to 100 nM.

As numbered in Table I. Compounds 13, 22, 24-25, 27, 34, 36-39, 42, 60,71, 77, 106, 111-112, 114, 117-118, 131, 148, 174, have an IC₅₀ in the P13-alpha assay of greater than 100 nM but less than or equal to 500nM.

As numbered in Table I, Compounds 17, 19, 33, 68-69, 109-110 have an IC₅₀ in the P13K-alpha assay of greater than 500 nM but less than or equal to 2500 nM.

### Embodiments 1

In one embodiment the invention comprises a compound of the invention having a P13K-alpha-inhibiiory activity of about 0.5 μM or less and is inactive for mTOR (when tested at a concentration of 2.0 μM or greater) or is selective for P13K-alpha over mTOR by about 5-fold or greater, about 7-fold or greater, or about 10-fold or greater. In another embodiment, the inveniion comprises a compound of the invention having a P13K-alpha-inhibitory activity of about 0.35 μM of less and is inactive for mTOR (when tested at a concentration of 2.0 μM or grecater) or is selective for P13K-alpha over mTOR by about 5-fold or greater, about 7-fold or greater, or about 10-fold or greater. In another embodiment, the inveniion comprises a compound of the invention having a P13K-alpha-inhibilory activity of about 0.25 μM or less and is inactive for mTOR (when tested at a concentration of 2.0 μM or greater) or is selective for P13K-alpha over mTOR by about 5-fold or greater, about 7-fold or greater, br about 10-fold orgreatcr. In another embodiment ihe compounds of the inveniion have an P13K-alpha-inhibitoryaclivily of about 0.1 μM or less and is inactive for mTOR (when tested at a concentration of 2.0 μM or greater) or is selective lor P13K-alpha over mTOR by about 5-fold or greater, about 7-fold of greater, or about 10-fold or greater. In another embodiment the invention comprises a compound of the invention having an P13K-alpha-inhibitory activity of about 0.05 μM or less and is selective for P13K-alpha over mTOR by about 5-fold or greater, about 7-fold or greater, or about 10-fold or greater.

### Emobidments 2

In one embodiment the invention comprises a compound of the invention having a P13K-alpha-inhibitory activity of about 2.0 μM or less and an mTOR-inhibitory activity of about 2.6 μM or less and the selectivity, for one of the targets over the other does not exceed 3-fold, In another embodiment the invention comprises a compound of the invention having a P13-alpha-inhibitory activity of about 1.0 μM or less and an mTOR-inhibitory activity of about 1.0 μM or less and the selectivity for one of the targets over the other does not exceed 3-fold. In another embodiment the invention comprises a compound of the invention having a P13K-alpha-inhibitory activity of about 0.5 μM or less and an mTOR-inhibitory activity of about 0.5 μM or less and the selectivity for one of the targets over the other does not exceed 3-fold. In another embodiment the invention comprises a compound of the invention haying a P13K-alpha-inhibitory activity of about 0.3 μM or less and an mTOR-inhibitory activity of abpout 0.3 μM or less and the selectivity for one of the targets over the other does not exceed 3-fold. In another embodiment the invention comprises a compound of the invention having a P13K-alpha-inliibiiory activity of about 0.2 μM or less and an mTOR-inhibitory activity of about. 0.2 μM or less and the selectivity for one of the targets over the other does not exceed 2-fold. In another embodiment the invention comprises a Compound of the invention having a P13K-alpha-inhibitory activity of about 0.15 μM or less and an mTOR-inhibitory activity of about 0.15 μM or less and the selectivity for one of the targets over, the other does not exceed 2-fold. In another embodiment the invention comprises a compound of the invention having a P13K-alpha-inhibitory activity of about 0.1 μM or less and all mTOR-inhibitory activity of about 0.1 μM or less. In another embodiment the invention comprises a compound of the invention having a P13K-alpha-inhibitory acitvitly of about 0.05 μM or less and an mTOR-inhibitory activity of about 0.05 μM of less. In another embodiment the invention comprises a compound of the invention have a P13K-alpha-inhibitory activity of about 0.02 μM or less and an mTOR-inhibitory activity of about 0.02 μM or less. In another embodiment the invention comprises a compound of the invention have a P13K-alpha-inhibitory activity of about 0.01 μM or less and and mTOR-inhibitory activiiy of about 0.01 μM or less.

### Biological Example 5

### pS6 (S240/244) ELISA Assay.

MCF-7 cells (ATCC) cells were seeded at 24000 cells per well in 96-well plates (Corning, 3904) in DMEM (Cellgro) containing 10% FBS (Cellgro), 1% NEAA (Cellgro) and 1% penicillin-streptomycin (Cellgro). Cells were incubated at 37° C. 5% CO₂ for 48 h. and the growth medium was replaced with serum-free DMEM or in medium containing 0.4% BSA. Serial dilutions of the lest Compound in 0.3% DMSO (vehicle) were added to the cells and incubated for 3 h. To fix the/cells, medium was removed and 100 μL/well of 4% formaldehyde (Sigma Aidrich, F8775) in TBS (20 mM Tris, 500 mM NaCl) was added to each well at RT for 30 min. Cells were washed 4 times widi 200 μL TBS containing 0.1% Triton X-1.00 (Sigma, catalog #T9284). Plates were blocked with 100 μL Odyssey blocking buffer (Li-Cor Biosciences, 927-40000) for 1 h at RT. Anti-pS6 (S240/244) antibody (Cell Signaling Technology, 2215) and anti-total-S6 antibody (R & D systems, MAB5436) were diluted 1:400 in Odyssey blocking buffer, and 50 μL of the antibody solution containing both antibodies was added io one plate to detect pS6 and total S6. After incubation overnight at 4° C., plates were washed 4 times with 200 μL TBS containing 0.1% Tween20 (Bio-Rad. catalog #170-6351) (TBST), Goat anti-rabbit and Goat anti-mouse secondary antibody (Li-Cor Biosciences, catalog #926-32221 and 926-32210) conjugated to IRDye were diluted 1:400 in Odyssey blocking buffer containing 0.1 % Twecn20, 50 μL of antibody solution containing both antibodies was added to each well and incubated for 1 h at RT. Plates were washed 3 times with 200 μL TBST and 2 times with 200 μL TBS. Fluorescence was read on an Odyssey plate reader. IC50 values were determined based on the ratio of pS6 to total S6 signal for Compound-treated wells, normalized to the DMSO-treated control wells.

In one embodiment, the Compounds of the Invention tested in this assay in MGF-7 cells had an inhibitory activity of 1.5 μM or less. In another embodiment, the Compounds of the Invention tested in this assay in MCE-7 cells had an inhibitory activity of 1.0 μM or less. In another embodtiment, the Compounds of the Invention tested in this assay in MCF-7cells had an inhibitory activity of 0.5 μM or less. In one embodiment, the Compounds, of the Invention tested in Ibis assay in MCF-7 cells had an inhibitory activity of 0.3 μM or less. In one embodiment, the Compounds of the Invention tested in this assay in MCF-7 cells had an inhibitory activity of 0.1 μM or less. In one embodiment, the Confounds of the Invention tested in this assay in MCF-7 cells had an inhibitory activity of 0.03 μM or less.

In one embodiment, the Compound of the Invention tested in this assay in PC-3cells had an inhibitory activity of about 1.7 μM or less. In another embodiment, the Compound of die Invention tested in this assay in PC-3 cells had an inhibitory activity of about 0.55 μM or less. In another embodiment, the Compound of the Invention tested in this assay in PC-3 cells had an inhibitory activity of about 0:55 μM or less. In another embodiment, the Compound of the Invention tested in this assay in PC-3 cells had an inhibitory activity of about 0.3 μM or less. In another embodiment, the Compound of the Invention tested in this assay in PC-3 cells had an inhibitory activity of about 0.1 μM or less. In another embodiment, the Compound of the Invention tested in this assay in PC-3 cells had an inhibitory activity of aboui0.05 μM or less.

### Biological Example 6

### pAKT (T308) ELISA Assay

MCF-7 cells (ATCC) cells were seeded at 24000 cells per well in 96- well plates (Corning, 3904) in DMEM (Cellgro) containing 10% FBS (Cellgro), 1% NEAA (Cellgro) and 1% penicillin-streptomycin (Cellgro). Cells were incubated at 37° C., 5% CO02 for 48 h, and the growth medium was replaced with serum-free DMEM or in medium containing 0.4% BSA. Serial dilutions of the test Compound in 0.3% DMSO (vehicle) were added to the cells and incubated for 3 h. At the end of the incubation period, cells were stimulated for 10 minutes by the addition of L-IGF (Sigma, I-1271) at a final concentration of 100 ng/ml. Afterwards, media was discarded from cell plates and 110 μl/well of cold lysis buffer (see table below) were added. Cell plates were incubated on ice and then put on shaker in 4° C. cold room for 1 h. Two capture plates (Thermo Scientific, Reacti-bind plate, 15042) were prepared for each cell plate by pre-coating with capture Akt antibody from the two sandwich ELISA antibody pairs used (Cell Signaling Technology 7142 and 7144). The Akt capture antibodies were diluted 1:100 in PBS and 100 μl of diluted capture antibody was added per well. Capture plates were incubated at 4C overnight. Prior to use, capture plates were washed 3 limes in TBS containing 0.1 % Tween20 (Bio-Rad, 170-6351) (TBST) and blocked in blocking buffer (Thermo Scientific, Starting Block T20, 37543) for 1-2 h at room temperature. After 1 h of cell lysis, 85 μl of cell lysale/well was transferred to the capture plate for detection of pAkt(T308). 15 μl of cell lysate was transferred from same well to the second capture plate for detection of total Akt1. After incubation overnight at 4° C. plates were washed 3 times with 200 μL TBST. Primary antibodies, diluted 1:100 in blocking buffer, were added to the corresponding capture plates for pAkt(T308) (Cell Signaling Technology, 7144) and total Akt1 (Cell Signaling Technology, 7142) detection and incubated at room temperature for 1 h. Plates were washed 3 times with 200 μL of TBST. Goal anti-mouse secondary antibody (Cell Signaling Technology, 7076) conjugated to MRP was diluted 1:1000 in blocking buffer and 100 μl were added to each well and incubated for 30 minutes at room temperature. Plates were then washed 3 times with 200 μL of TBST. 100 μL of SuperSignal ELISA Femto stable peroxidase solution (Thermo Scientific, 37075) was added to each well. After 1 minute incubation, chemiluminescence was read on a Wallac Victor2 1420 multilabel counter. IC50 values were determined based on the ratio of pAkt(T308) to total Akt1 signal for Compound treated wells, normalized to the DMSO-treated control wells.

| | Stock | Final | /10 mL | |
|---|---|---|---|---|
| Water | | | 6 | mL |
| Complete Protease | | | 1 mini- | |
| Inhibitors (Roche | | | tablet | |
| 1 836 170) | | | | |
| 5x RIPA | 5x | 1x | 2 | mL |
| NaF | 200 mM | 1 mM | 50 | μL |
| B-glycerophosphate | 100 mM | 20 mM | 1.8 | mL |
| Phosphatase Inhibitor I | 100x | 1x | 100 | μL |
| (Sigma P2850) | | | | |
| Na orthovanadate | 200 mM | 1 mM | 50 | μL |
| EDTA, pH 8 | 500 mM | 1 mM | 20 | μL |

In one embodiment, the Compounds of the Invention tested in this assay in PC-3cells had an inhibitory activity of about 2.0 μM or less. :In another embodiment, the Compounds of the Invention tested in this assay in PC-3 cells had an inhibitory activity of about 1.0 μM or less. In another embodiment, the Compounds of the Invention tested in this assay in PC-3 cells had an inhibitory activity of about 0.3 μM or less. In another embodiment, the Compounds of the Invention tested in this assay in PC-3 cells had an inhibitory activity of about 0.2 pM or less.

In one embodiment, the Compounds of the Invention tested in this assay in MCF-7 cells had an inhibitory activity of about 3.0 μM or less. In another embodiment, the Compounds of the Invention tested in this assay in MCF-7 cells had an inhibitory activity of about 3.0 μM or less. In another embodiment, the Compounds of the Invention tested in this assay in MCF-7 cells had an inhibitory activity of about 1.5 μM or less. In another embodiment, the Compounds of the Invention tested in this assay in MCF-7 cells had an inhibitory activity of about 0.75 μM or less. In another embodiment, the Compounds of the Invention tested in this assay in MCF-7 cells had an inhibitory activity of about 0.5 μM or less. In another embodiment, the Compounds of the Invention tested in this assay in MCF-7cells had an inhibitory activity of about 0.25 μM or less. In another embodiment, ihe Compounds of the Invention tested in this assay in MCF-7 cells had an inhibitory activity of about 0.1 μM or less.

### Biological Example 713

### Pharmacodynamic Xenograft Tumor Models

Female and male athymic nude mice (NCr) 5-8 weeks of age and weighing approximately 2025 g are used, in the following models. Prior to initiation of a study, the animals are allowed to aeclimate for a minimum of 48 h. During these studies, animals are provided food and water ad libitum and housed in a room condition at 70-75° F. and 60% relative huhtidity.A 12 h light and 12 h dark cycle is maintained with automatic timers. All animals are examined daily for compound-induced or tumor-related deaths.

### MCF-7 Breast Adenocarcinoma Model

MCF7 human mammary adenocarcinoma cells are cultured in vitro in DMEM (Cellgro) supplemented with 10% Fetal Bovine Serum (Cellgro), Penicillin-Streptomycin and non-essential amino acids at 37° C. in a humidified 5% CO₂ atmosphere. On day 0, cells are harvested by trypsinization, and 5×10⁶ cells in 100 μL of a solution made of 50% cold Hanks balanced salt solution with 50% growth factor reduced matrigel (Beeton Dickinson) implanted subcutancously into the hindflahk of female nude mice. A transponder is implanted into each mouse for identification and data tracking, and animals are moonitored daily for clinical symptoms and survival.

Tumors are established in female athymic nude mice and staged when the average tumor weight reached 100-200 mg A Compound of the Invention is orally administered as a solinion/fine suspension in water (with 1:1 molar ratio of 1 N NCL) once-daily (qd) or twice-daily (bid) at 10, 25, 50 and 100 mg/kg for 14days. During the dosing period of 14-19 days, tumor weights are determined twice-weekly and body weights are recorded daily.

### Colo-205 Colon Model

Colo-205 human colorectal carcinoma cells are cultured in vitro in DMEM (Mediatech) supplemented with 10% Fetal Bovine Serum (Hyclone), Penicillin-Streptomycin and non-essential amino acids at 37° C. in a humidified. 5% CO₂ atmosphere. On day 0, cells are harvested by trypsinization, and 3×10⁶ cells (passage 10-15, >95% viability) in 0.1 mL ice-cold Hank's balanced salt solulion are implanted intradermally in the hind-flank of 58 week old female albymic nude mice. A transponder is implanted in each mouse for identification, and animalsare monitored daily for clinical symptoms-and survival.

Tumors are established in female athymic nude mice and staged when the average tumor weight reached 100-200 mg. A Compound of the Invention is orally administered as a solution/fine suspension in water (with 1:1 molar ratio of 1 N HCL) once-daily (qd) or twice-daily (bid) al 10, 25, 50 and 100 mg/kg for 14 days. During the dosing period of 14 days, tumor weights are determined twice-weekly and body weights are recorded daily.

### PC- 3 Prostate Adenocarcinoma Model

PC-3 human prostate adenocarcinoma cells are cultured in vitro in DMEM (Mediatech) supplemented with 20% Fcial Bovine Serum (Hyclone), Penicillin-Streptomycin and non-essential amino acids at 37° C. in a humidified 5% CO₀₂ atmosphere. On day 0, cells are harvested by trypsinization and 3×10⁶ cells (passage 10-14, >95% viability) in 0.1 mL of ice-cold flank's balanced salt solution are implanted subcutancously into the hindflank of 5-8 week old male nude mice. A transponder is implanted in each mouse for identification, and animals are monitored daily for clinical symptoms and survival.

Tumors are established in male athymic nude and staged when the average tumor weight reachcd 100-200 mg. A Compound of the Invention is orally administered as a solution/fine suspension in water (with 1:1 molar ratio of 1 HCl ) once-daily (qd) or twice-daily (bid) at 10, 25, 50, or 100-mg/kg for 19 days. During the closing period of 14-19 days, tumor weights are determined twice-weekly and body weights are recorded daily.

### U-87 MG Human Glioblastoma Model

U-87 MG human glioblastoma cells are cultured in vitro in DMEM (Mediatech) supplemented with 10% Fetal Bovine Seruni,(Hyclone), Penicillin-Strepiomyein and non-essential amino acids at 37° C. in a humidified 5% OC₂ atmosphere. On day 0, cells are harvested by trypsinization and 2×10⁶ cells (passage 5, 96% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A549 Human Lung Carcinoma Model

A549 human lung carcinoma cells are cultured in vitro in DMEM (Mediatech) supplemented with 10% Fetal Bovine Serum (Hyclone), Penicillin-Streptomycin and non-essential amino acids at 37° C. in a humidified 5% CO₂ atmosphere. On day 0, cells are harvested by trypsinization and 10×10⁶ cells (passage 12.9% viability) in 0.1 mL of ice-cold Hank's balanced salt solution are implanted intradermally into the hindflank of 5-8 week old female nude mice. A transponder is implanted in each mouse for identification, and animals are monitored daily for clinical symptoms and survival. Body weights are recorded daily.

### A2058 Human Melanoma Model

A2058 human melanoma cells are cultured in vitro in DMEM (Mediatech) supplemented with 10% Fetal Bovine Scrum (Hyclone), Penicillin-Streptomycin and non-essential amino acids at 37° C. in a humidified. 5% CO₂ atmosphere. On day 0, cells are harvested by trypsinization and 3×10⁸ cells (passage 3, 95% viability) in 0.1 mL ice-cold Hank's balanced salt solution are implanted intradermally in the hind-flank of 5-8 week old female athymic nude mice. A transponder is implanted in each mouse for identification, and animals are monitored daily for clinical syihptoms and survival. Body weights, are recorded daily.

### WM-266-4 Human Melanoma Model

WM-266-4 human melanoma cells are cultured in vitro in DM EM (Mediatech) supplemented with 10% Fetal Bovine Scrum (Hyclone). Penicillin-Streptomycin and non-essential amino acids at 37° C. in a humidified, 5% CO₂ atmosphere. On-day 0, cells are harvestedby trypsinization and 3×106 cells (passage 5, 99% viability), in 0.1 mL ice-cold Hank's balanced salt solution are implanted intradermally in the hind-flank of 5-8 week old female athymic nude mice, A transponder is implanted inieaeh mouse for identification, and animals are monitored daily for clinical symptoms and survival. Body weights are recorded daily.

Tumor weight (TW) in the above models is determined by measuring perpendicular diameters with a caliper, using the following formula:

tumor weight (mg)=[tumor volume=length (mm)=width² (mm²)]/2

These data were recorded and plotted on a tumor weight vs. days post-implantation line graph and presented graphically as an indication of tumor growth rates. Percent inhibition of tumor growth (TGI) is determined with the following formula:

$$[ 1 - ( \frac{( {X}_{f} - {X}_{0} )}{( {Y}_{f} - {X}_{0} )} ) ] * 100$$

where X₀ = average TW of all tumors on group day

Xᵣ=TW of treated group on Day f

Yᵣ=TW of vehicle control group on Day f

If tumors regress below their starting sizes, then the percent tumor regression is determined with the following formula:

$$( \frac{{X}_{0} - {X}_{f}}{{X}_{0}} ) * 100$$

Tumor size is calculated individually for each tumor to obtain a mean ±SEM value for each experimental group. Statistical significance is determined using, the 2-tailed Student's t-test (significance defined as P<0.05).

The foregoing invention has been described in some detail by way of illustration and example. For purposes of clarity and understanding.The invention has been described with reference to various specific embodiments andtcehniqucs. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore,be determined not with reference to the above descriplion, but should instead be determined with reference to the following appended claims, along with the full seopeof equivalents to which such claims are entitled. All patents, patent applications and publications cited in this application are hereby incorporated by reference in their entirety for all purposes to the same extent as if each individual patent, patent application or publication were so individually denoted.

What is claimed:

1. A Compound of Formula I: 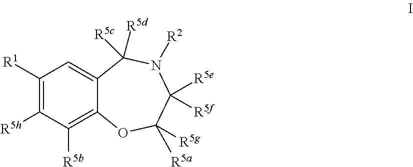
or a single stereoisomer or mixture of stereoisomers thereof and additionally optionally as a pharmaceutically acceptable salt thereof, where
R¹ is phenyl optionally substituted with one, two, or three R⁶ groups; or
R¹ is heteroaryl optionally substituted with one, two, or three R⁷;
R² is heteroaryl substituted with R³, R³a, R³b, R³c, and R³d;
R³, R³a, R³b, R³c, and R³d are independently hydrogen, cyano, nitro, alkyl, alkenyl, alkynyl, halo, haloalkyl, hydroxyalkyl, alkoxyalkyl,
cyanoalkyl, —SR¹², —S(O)₂R²⁰, —C(O)H, —C(O)OR⁴, —C(O)NHR⁴, halocarbonyl, —NR¹¹R^{11a}, —OR^{11a}, optionally substituted phenyl, optionally substituted phenylalkyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, or alkyl substituted with one or two R¹⁶; or
two of R³, R^{3a}, R^{3b}, R^{3c}, and R^{3d}, when attached to the same carbon, form an optionally substituted cycloalkyl, optionally substituted aryl, or an optionally substituted heterocycoalkyl, or optionally substituted heteroaryl, and the other of R³, R^{3a}, R^{3b}, R^{3c}3, and R³d are independently hydrogen, cyano, nitro, alkyl, alkenyl, alkynyl, halo, haloalkyl, hydroxyalkyl, alkoxyalkyl, cyanoalkyl, —SR¹², —S(O)₂R²⁰, —C(O)H, —C(O)OR⁴, halocarbonyl, —C(O)NHR⁴, halocarbonyl, —NRuR^{11a}, —OR^{11a}, optionally substituted phenyl, optionally substituted phenylalkyl, optionally substituted cycloalkyl, optionally substituted cycloalkylalkyl, optionally substituted heterocycloalkyi, optionally substituted heterocycloalkylalkyl, optionally substituted heteroaryl, optionally substituted heteroarylalkyl, or alkyl substituted with one or two R¹⁶;
R⁴ is alkyl, alkenyl, alkynyl, hydroxyalkyl, alkoxyalkyl, haloalkyl, aminoalkyl, alkylaminoalkyl, dialkylaminoalkyl, benzyl, or optionally substituted heterocycloalkylalkyl;
R^{5a} and R^{5c} are independently hydrogen, deuterium, or alkyl;
R^{5h} is hydrogen, deuterium or halo;
R^{5b} is deuterium, (C₁₋₃)alkyl, (C₁₋₃)alkoxy, halo(C₁₋₃)alkyl, or (C₁₋₃)haloalkoxy;
R^{5d}, R^{5e}, R^{5f}, and R^{5g} are hydrogen or deuterium;
each R⁶, when R⁶ is present, is independently nitro; cyano; halo; alkyl; alkenyl; alkynyl; haloalkyl; —OR^{8a}; —NR⁸R⁸a; —C(O)NR⁸R^{8a}; —S(O)₂R⁸; —NR^{8C}(O)OR⁹; —NR^{8C}(O)R⁹; —NR⁸S(O)_{2 R}^{8a}; —NR⁸C(O)NR^{8a}R⁹; carboxy, —C(O)OR⁹; halocarbonyl; alkylcarbonyl; alkyl substituted with one or two —C(O)NR⁸R^{8a}; heteroaryl optionally substituted with 1, 2, or 3 R¹⁴; or optionally substituted heterocycloalkyl; or
two R⁶, together with the carbons to which they are attached, form an optionally substituted 3, 4, 5, or 6-membered cycloalkyl or heterocycloalkyl;
each R⁷, when R⁷ is present, is independently oxo; nitro; cyano; alkyl; alkenyl; alkynyl; halo; haloalkyl; hydroxyalkyl;
alkoxyalkyl; —OR^{8a}; —SR¹³; —S(O)R¹³; —S(O)₂R^{13a}; —NR⁸R^{8a}; —C(O)NR⁸R^{8a}; —NR⁸C(O)OR⁹; —N R⁸C(O)R⁹; —NR⁸S(O)₂R^{8a}; —NR⁸C(O)NR^{8a}R⁹; —C(O)OR⁹; halocarbonyl; alkylcarbonyl; —S(O)₂NR⁸R⁹; alkylsulfonylalkyl; alkyl substituted with one or two —NR⁸R^{8a}; alkyl substituted with one or two —NR⁸C(O)R^{8a}; alkyl substituted with one or two —NR⁸C(O)OR⁹; alkyl substituted with one or two —S(O)₂R^{13a}; optionally substituted cycloalkyl; optionally substituted cycloalkylalkyl; optionally substituted heterocycloalkyl; optionally substituted heterocycloalkylalkyl; optionally substituted phenyl; optionally substituted phenylalkyl; optionally substituted heteroaryl; or optionally substituted heteroarylalkyl;
each R⁸, R¹¹, R¹⁵, R¹⁷, and R¹⁸ are independently hydrogen, NH₂, NH(alkyl), N(alkyl)₂, alkyl, alkenyl, alkynyl, hydroxyalkyl, alkoxyalkyl, or haloalkyl;
each R^{8a}, R^{11a},and R^{15a} are independently hydrogen, alkyl, alkenyl, alkynyl, haloalkyl, hydroxyalkyl, cyanoalkyl, aminoalkyl, alkylaminoalkyl, dialkylaminoalkyl, alkoxyalkyl, carboxyalkyl, optionally substituted cycloalkyl, optionally substituted cycloalkylalkyl, optionally substituted heterocycloalkyi, optionally substituted heterocycloalkylalkyl, optionally substituted phenyl, optionally substituted phenylalkyl, optionally substituted heteroaryl, or optionally substituted heteroarylalkyl;
R⁹ is hydrogen; alkyl; alkenyl; alkynyl; hydroxyalkyl; alkoxyalkyl; aminoalkyl; alkylaminoalkyl; dialkylaminoalkyl; haloalkyl; hydroxyalkyl substituted with one, two, or three groups which are independently halo, amino, alkylamino, or dialkylamino; alkyl substituted with one or two aminocarbonyl; optionally substituted phenyl; optionally substituted phenylalkyl; optionally substituted cycloalkyl; optionally substituted cycloalkylalkyl; optionally substituted heteroaryl; optionally substituted heteroarylalkyl; optionally substituted heterocycloalkyl; or optionally substituted heterocycloalkylalkyl;
R¹² is alkyl or optionally substituted phenylalkyl;
R¹³ is alkyl, hydroxyalkyl, or haloalkyl; and
R^{13a} is hydroxy, alkyl, haloalkyl, hydroxyalkyl, or heterocycloalkyl optionally substituted with one or two groups which are independently halo, amino, alkylamino, dialkylamino, hydroxy, alkyl, or hydroxyalkyl;
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbonyl, alkylaminocarbonyl, dialkylaminocarbonyl, or optionally substituted phenyl;
each R¹⁶ is independently halo, —NR¹¹R^{11a}, —NR¹⁵S(O)R^{15a}, —OC(O)R¹⁷, carboxy, alkoxycarbonyl, —NHC(O)R^{15a}, or —OR¹⁸; and
R²⁰ is alkyl, haloalkyl, hydroxyalkyl, amino, alkylamino, dialkylamino, or heterocycloalkyl; and with the proviso that if one of R^{5a}, R^{5c}, R^{5d}, R^{5e}, R^{5f}, R^{5g}, and R^{5h} are deuterium, then R^{5b}
is H, (C₁₋₃)alkyl or halo(C₁₋₃)alkyl.

2. The compound of claim 1, wherein the compound is a compound of Formula I(a) 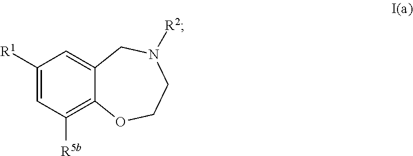
or a single stereoisomer or mixture of stereoisomers thereof and additionally optionally as a pharmaceutically acceptable salt thereof.

3. The compound of claim 2, or a single stereoisomer or mixture of stereoisomers thereof and additionally optionally as a pharmaceutically acceptable salt thereof, where
R¹ is phenyl optionally substituted with one, two, or three R⁶ groups; or
R¹ is heteroaryl optionally substituted with one, two, or three R⁷;
R² is heteroaryl substituted with R³, R^{3a}, R^{3b}, R^{3c}, and R^{3d};
R³, R^{3a}, R^{3b}, R^{3c}, and R^{3d} are independently hydrogen; cyano; alkyl; alkenyi; halo; haloalkyl; hydroxyalkyl; alkoxyalkyl; cyanoalkyl; —SR¹²; —S(O)₂R²⁰; carboxy; alkoxycarbonyl; halocarbonyl; —NR¹¹R^{11a}; —OR^{11a}; phenyl optionally substituted with one or two groups which are independently alkyl or halo; phenylalkyl optionally substituted with one or two R¹⁹; cycloalkyl; cycloalkylalkyl; heterocycloalkyl optionally substituted with one or two groups which are independently alkyl, alkoxycarbonyl, or benzyloxycarbonyl; heterocycloalkylalkyl optionally substituted with one or two groups which are independently alkyl, alkoxycarbonyl, or benzyloxycarbonyl; heteroaryl; heteroarylalkyl; or alkyl substituted with one or two R¹⁶; or
two of R³, R^{3a}, R^{3b}, R^{3c}, and R^{3d}, when attached to the same carbon, form a cycloalkyl or a heterocycoalkyl; and the other of R³, R^{3a}, R^{3b}, R^{3c}, and R^{3d} are hydrogen;
each R⁶, when R⁶ is present, is independently nitro; cyano; halo; alkyl; halo; haloalkyl; —OR^{8a}; —NR⁸R^{8a}; —C(O)NR⁸R^{8a}; —S(O)₂R8; —NR⁸C(O)R⁹; —NR⁸s(O)₂R^{8a}; —NHC(O)N HR⁹; carboxy, —C(O)OR⁹; or heteroaryl optionally substituted with 1, 2, or 3 R¹⁴;
each R⁷, when R⁷ is present, is independently oxo; nitro; cyano; alkyl; alkenyl; halo; haloalkyl; hydroxyalkyl;
alkoxyalkyl; —OR^{8a}; —SR¹³; —S(O)R¹³; —S(O)₂R^{13a}; —NR⁸R^{8a}; —C(O)NR⁸R^{8a}; —NR⁸c(O)OR⁹; —N R^{8C}(O)R⁹; —NR⁸S(O)₂R^{8a}; —NR^{8C}(O)NR^{8a}R⁹; —C(O)OR⁹; halocarbonyl; —S(O)₂NR⁸R⁹; alkylsulfonylalkyl; alkyl substituted with one or two —NR⁸R^{8a}; alkyl substituted with one or two —NR⁸C(O)R^{8a}; alkyl substituted with one or two —NR⁸c(O)OR⁹; alkyl substituted with one or two —S(O)₂R^{13a}; cycloalkyl; cycloalkylalkyl; heterocycloalkyl optionally substituted with one or two groups which are independently alkyl or amino; phenyl; phenylalkyl; heterocycloalkylalkyl; heteroaryl; or heteroarylalkyl;
R⁸, R¹¹, R¹⁵, R¹⁷, and R¹⁸ are independently hydrogen, alkyl, alkenyl, alkynyl, hydroxyalkyl, alkoxyalkyl, or haloalkyl;
R^{8a}; R^{11a}; and R^{15a} are independently hydrogen; alkyl; alkenyl; alkynyl; haloalkyl; hydroxyalkyl; cyanoalkyl; aminoalkyl; alkylaniinoalkyl; dialkylaminoalkyl; alkoxyalkyl; carboxyalkyl; cycloalkyl; cycloalkylalkyi; heteroeycloalkyl optionally substituted with one or two groups which are independently alkyl, alkoxycarbonyl, or benzyloxy; heterocycloalkylalkyl optionally substituted with one or two groups which are independently alkyl, alkoxycarbonyl, or benzyloxy; phenyl optionally substituted with one or two groups which are independently halo, alkyl, or alkoxy; phenylalkyl; heteroaryl; or heteroarylalkyl;
R⁹ is hydrogen; alkyl; alkenyl; alkynyl; hydroxyalkyl; alkoxyalkyl; aminoalkyl; alkylaniinoalkyl; dialkylaminoalkyl; haloalkyl; hydroxyalkyl substituted with one, two, or three groups which are independently halo, amino, alkylamino, or dialkylamino; alkyl substituted with one or two aminocarbonyl; phenyl; phenylalkyl; cycloalkyl; cycloalkylalkyi optionally substituted with one or two groups which are independently amino or alkyl; heteroeycloalkyl optionally substituted with one or two groups which are independently alkyl, alkoxycarbonyl, or benzyloxy; or heterocycloalkylalkyl optionally substituted with one or two groups which are independently alkyl, alkoxycarbonyl, or benzyloxy;
R¹² is alkyl or phenylalkyl;
R¹³ is alkyl, hydroxyalkyl, or haloalkyl; and
R^{13a} is hydroxy, alkyl, haloalkyl, hydroxyalkyl, or heteroeycloalkyl optionally substituted with one or two groups which are independently halo, amino, alkylamino, dialkylamino, hydroxy, alkyl, or hydroxyalkyl;
each R¹⁴, when R¹⁴ is present, is independently amino, alkylamino, dialkylamino, acylamino, halo, hydroxy, alkyl, haloalkyl, hydroxyalkyl, aminoalkyl, alkylaminoalkyl, dialkylaminoalkyl, alkoxycarbonyl, aminocarbonyl, alkylaminocarbonyl, dialkylaminocarbonyl, or phenyl;
each R¹⁶ is independently NR^{11R}^{11a}, —NR¹⁵S(O)R^{15a}, —OC(O)R¹⁷, or —OR¹⁸;
each R¹⁹ is independently halo, alkyl, haloalkyl, amino, alkylamino, dialkylamino, or alkoxy; and
R²⁰ is amino, alkylamino, dialkylamino, or heteroeycloalkyl.

4. The compound of claim 3, where R¹is phenyl optionally substituted with one, two, or three R⁶ groups; or a single stereoisomer or mixture of stereoisomers thereof and additionally optionally as a pharmaceutically acceptable salt thereof.

5. The compound of claim 4, where R¹ is phenyl substituted with one or two R⁶ groups independently nitro, halo, alkoxy, —OR^{8a}, —S(O)₂R⁸; —NR⁸R^{8a}, —NR⁸S(O)₂R^{8a}, —NR⁸C(O)R⁹, —C(O)NR⁸R^{8a}, —NR⁸C(O)NR^{8a}R⁹, carboxy, alkoxycarbonyl, or heteroaryl optionally substituted with one or two R¹⁴; or a single stereoisomer or mixture of stereoisomers thereof and additionally optionally as a pharmaceutically acceptable salt thereof.

6. The compound of claim 5, where R¹ is phenyl substituted with one R⁶ where R⁶ is —S(O)₂R⁸, —C(O)NR⁸R^{8a} or heteroaryl optionally substituted with one or two R¹⁴; or a single stereoisomer or mixture of stereoisomers thereof and additionally optionally as a pharmaceutically acceptable salt thereof.

7. The compound of claim 2, where R¹ is heteroaryl optionally substituted with one, two, or three R⁷; or a single stereoisomer or mixture of stereoisomers thereof and additionally optionally as a pharmaceutically acceptable salt thereof.

8. The compound of claim 7, where R¹ is a 9-membered heteroaryl optionally substituted with one, two, or three R⁷; or a single stereoisomer or mixture of stereoisomers thereof and additionally optionally as a pharmaceutically acceptable salt thereof.

9. The compound of claim 8, where R¹ is a 9-membered heteroaryl and the 9-membered heteroaryl is benzimidazolyl, 1H-imidazo[4,5-b]pyridinyl, 3H-imidazo[4,5-b]pyridinyl, 1H-imidazo[4,5-c]pyridinyl, 3H- imidazo[4,5-c]pyridinyl, thiazolo[4,5-b]pyridinyl, thiazolo[4,5-c]pyridinyl, thiazolo[5,4- c]pyridinyl, or thiazolo[5,4-b]pyridinyl where R¹ is optionally substituted with one, two, or three
R⁷; or a single stereoisomer or mixture of stereoisomers thereof and additionally optionally as a pharmaceutically acceptable salt thereof.

10. The compound of claim 7, where R¹ is a 5-membered heteroaryl optionally substituted with one, two, or three R⁷; optionally where the 5-membered heteroaryl is thiazolyl or pyrazolyl and where the 5-membered is optionally substituted with one, two, or three R⁷; or a single stereoisomer or mixture of stereoisomers thereof and additionally optionally as a pharmaceutically acceptable salt thereof.

11. The compound of claim 7, where R¹ is a 6-membered heteroaryl optionally substituted with one, two, or three R⁷; optionally where the 6-membered heteroaryl is pyrimidinyl, pyridinyl, pyrazinyl, or pyridazinyl and where the 6-membered heteroaryl is optionally substituted with one, two, or three R⁷; or a single stereoisomer or mixture of stereoisomers thereof and additionally optionally as a pharmaceutically acceptable salt thereof.

12. compound of claim 11, where R¹ is pyridinyl optionally substituted with one, two, or three R⁷; or a single stereoisomer or mixture of stereoisomers thereof and additionally optionally as a pharmaceutically acceptable salt thereof.

13. The compound of claim 7, where R¹ is optionally substituted with one or two R⁷ where each R⁷, when R⁷ is present, is independently halo, alkyl, cycloalkyl, haloalkyl, hydroxyalkyl, alkoxyalkyl, —NR⁸R^{8a}, or —NR⁸C(O)OR⁹; or a single stereoisomer or mixture of stereoisomers thereof and additionally optionally as a pharmaceutically acceptable salt thereof.

14. The compound of claim 2, where R² is quinazolin-4-yl, quinolin-4-yl, isoquinolin—4—yl, ,6,7,8- tetrahvdroquinazolin-4-yl, 6,7-dihydro-5-cyclopenta[f]pyrimidin-4-yl, 6,7,8,9-tetrahvdro-5H- cyclohepta[d]pyrimidin-4-yl, 5,6-dihydroquinazolin-4-yl, 7′,8′-dihydro-5′H-spirorcyclopropane- 1,6′-quinazoline]-4′-yl, or 6′,8′-dihydro-5′H-spiro[cyclopropane-1,7′-quinazoline]-4′-yl wherein R²is substituted with R³, R^{3a}, R^{3b}, R^{3c}, and R^{3d}; where R^{3c} and R^{3d} are hydrogen; or a single stereoisomer or mixture of stereoisomers thereof and additionally optionally as a pharmaceutically acceptable salt thereof.

15. Cancelled.

16. Cancelled.

17. The compound of claim 2, where R² is according to formula (a) 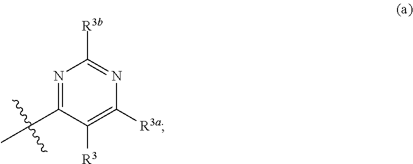
where R³ is hydrogen, halo, alkyl, cycloalkylalkyl, or phenylalkyl optionally substituted with one or two R¹⁹: R^{3a} is hydrogen, alkyl, halo, optionally substituted heterocycloalkyl or —NR¹¹R^{11a}; and R^{3b} is hydrogen, alkyl, hydroxyalkyl, or alkyl substituted with one or two R¹⁶; or a single stereoisomer or mixture of stereoisomers thereof and additionally optionally as a pharmaceutically acceptable salt thereof.

18. Cancelled.

19. Cancelled.

20. The claim 2 where R² is according to formula (g) 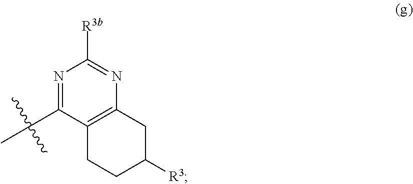
where R^{3b} is hydrogen, alkyl, alkenyl, hydroxyalkyl, cyanoalkyl, optionally substituted heterocycloalkyl, optionally substituted heterocycloalkylalkyl, or alkyl substituted with one R¹⁶;
or a single stereoisomer or mixture of stereoisomers thereof and additionally optionally as a pharmaceutically acceptable salt thereof.

21. The where R² is according to formula (d) 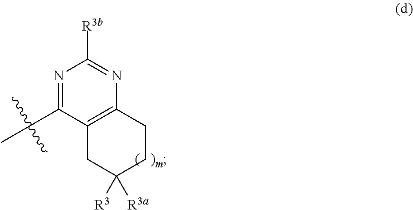
wherein m is 1;
where R^{3b} is hydrogen, alkyl, alkenyl, hydroxyalkyl, cyanoalkyl, optionally substituted heteorcycloalkyl, optionally substituted heteorcycloalkylalkyl, or alkyl substituted with one R¹⁶; and
where R³ and R^{3a} together with the carbon to which they are attached form an optionally substituted cycloalkyl; or where R³ and R^{3a} are halo or R³ and R^{3a} are alkyl;
or a single stereoisomer or mixture of stereoisomers thereof and additionally optionally as a pharmaceutically acceptable salt thereof.

22. Cancelled.

23. Cancelled.

24. Cancelled.

25. The compound of claim 2 where R² is according to formula (e) 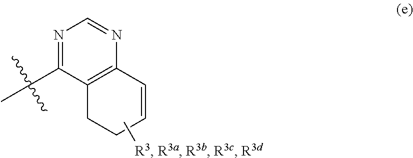
where R³, R^{3a}, R^{3b}, R^{3c}, and R^{3d} are positioned on any substitutable carbon on the ring of formula (e); or a single stereoisomer or mixture of stereoisomers thereof and additionally optionally as a pharmaceutically acceptable salt thereof.

26. The compound according to of claim lx which wherein the compound is a compound of formula II(a), II(b), III(a), III(b), IV(a), IV(b), V(a), V(b), V(c), V(d), VI(a), VI(a), or VII.

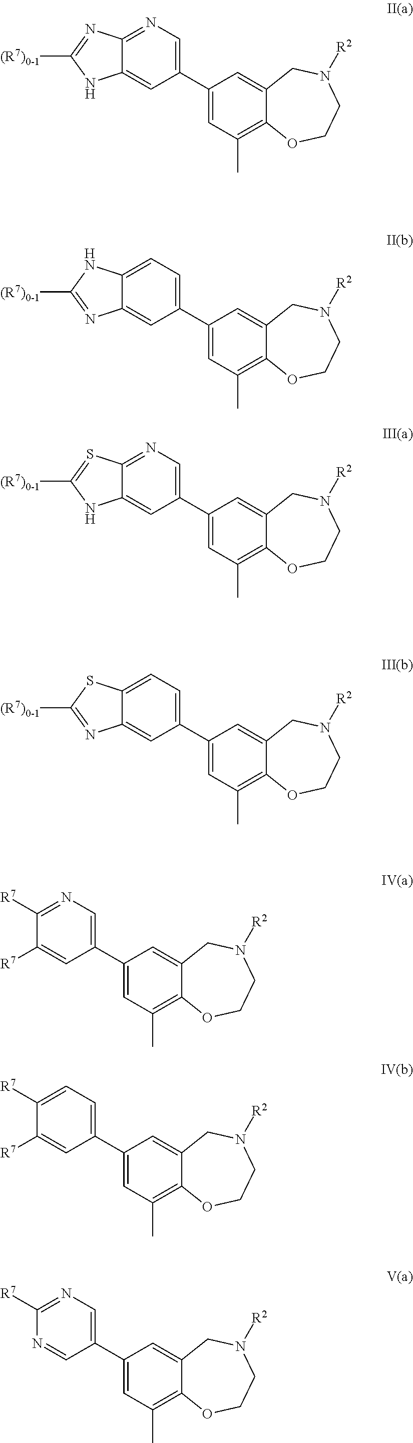

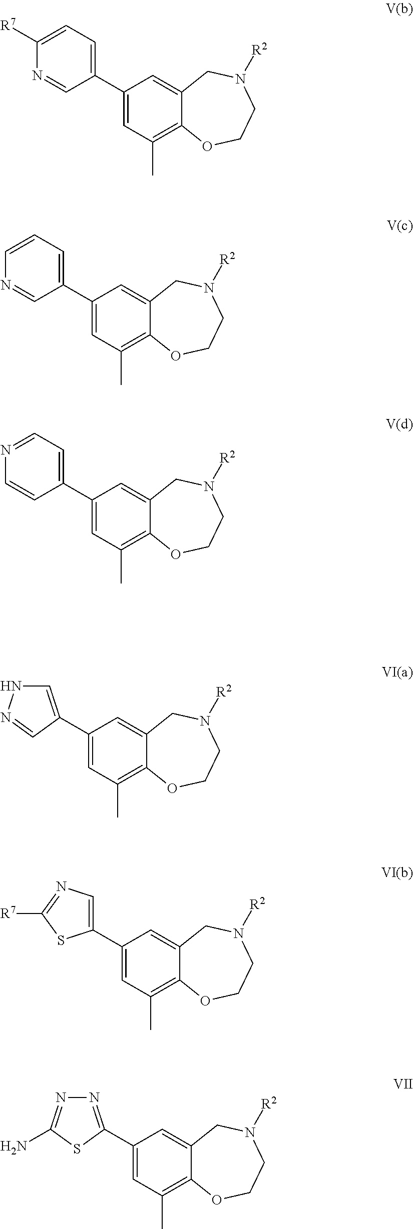

27. The compound of claim 26, wherein the compound is a compound of Formula II(a) or II(b) 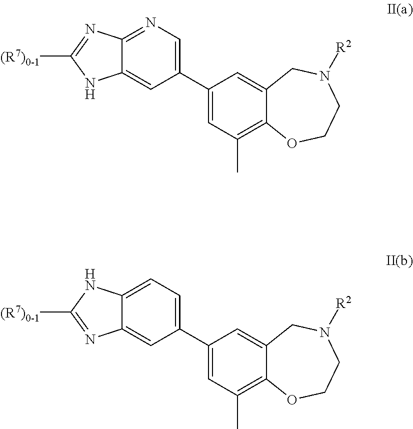
wherein R⁷ is methyl or NH₂; and 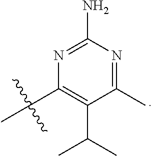 [SMILES: *c1nc(N)nc(C)c1C(C)C]
wherein R² is

28. Cancelled.

29. The compound of claim 26, wherein the compound is a compound of Formula III(a) or III(b) 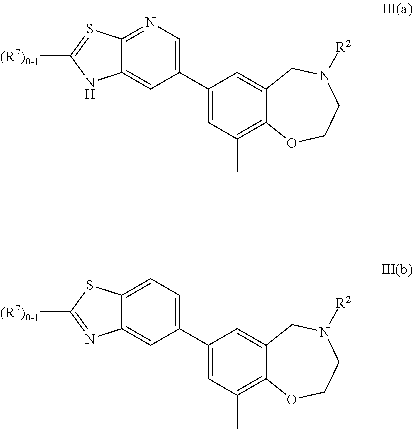
wherein R⁷ is methyl or NH₂; and 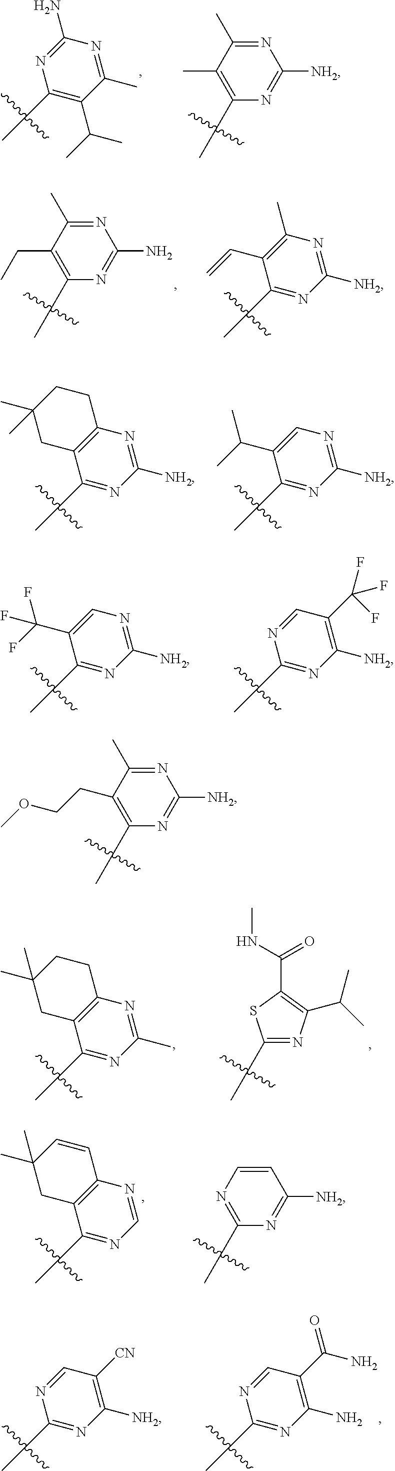 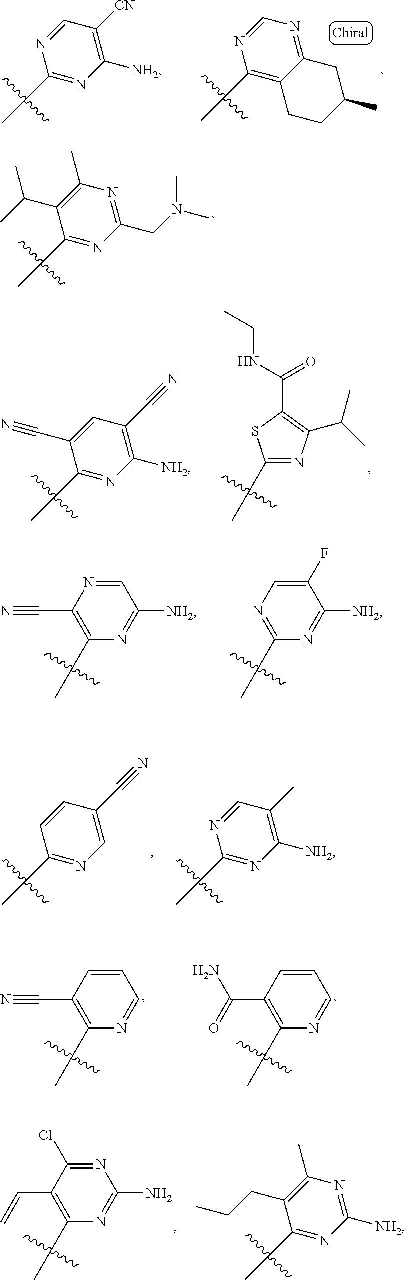 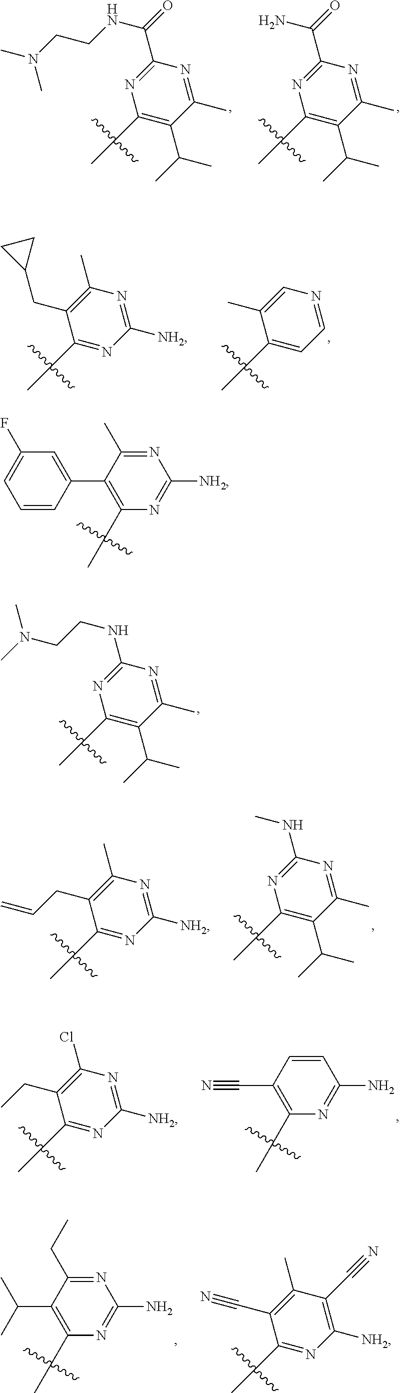 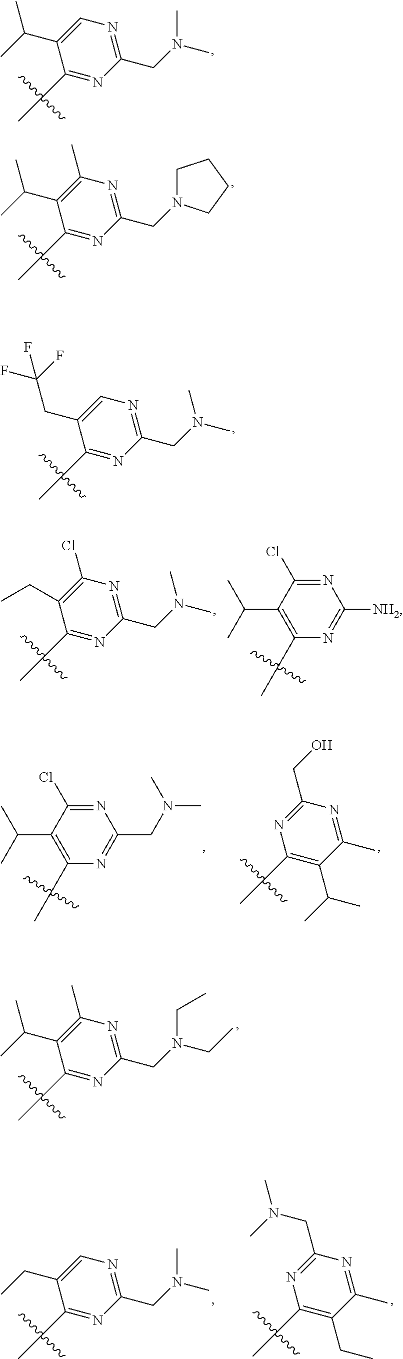 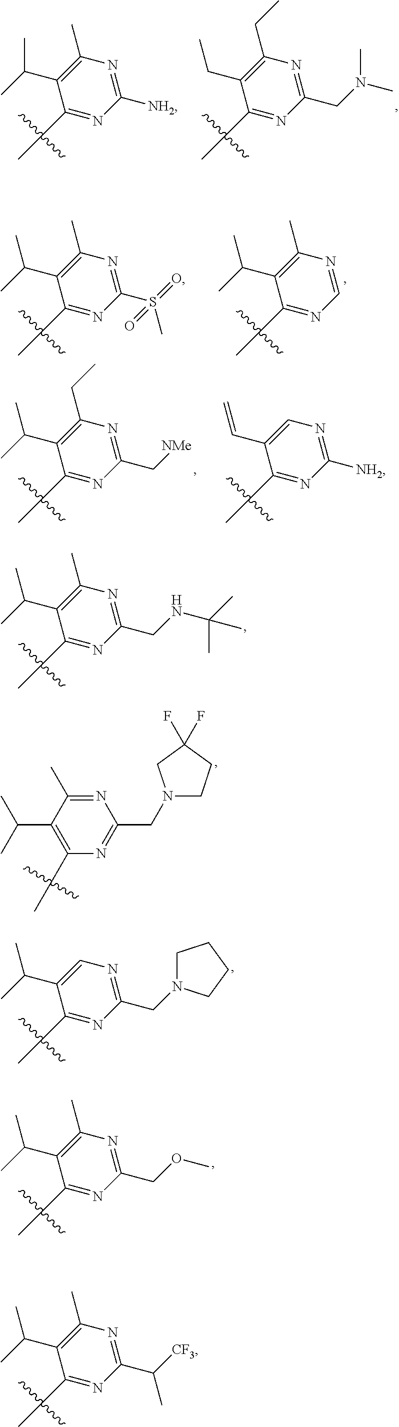 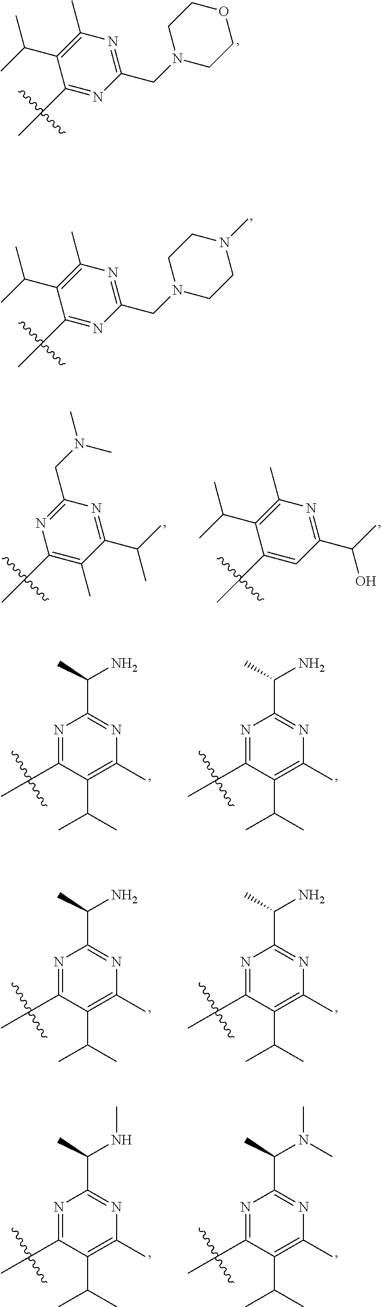 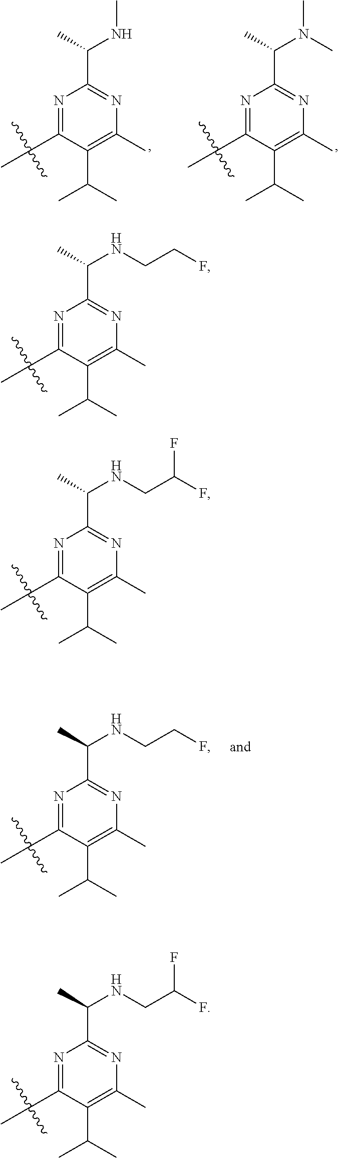
wherein R² is selected from the group consisting of

30. Cancelled.

31. The compound of claim 26, wherein the compound is a compound of Formula IV(a) or IV(b) 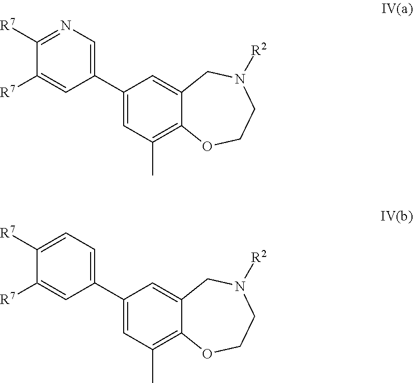
wherein one or both R⁷ groups are optionally present;
wherein when both R⁷ groups are present, one R⁷ is NH₂, chloro, hydroxy, —CO₂Me, or methoxy, and the other R⁷ is —SO₂NH₂, —NHSO₂Me, or methoxy; and 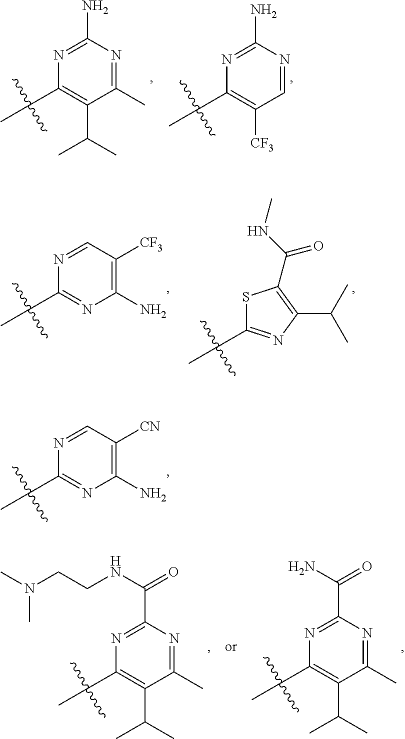
wherein R² is

32. Cancelled.

33. Cancelled.

34. The compound of claim 26, wherein the compound is a compound of Formula IV(a1) or IV(b1) 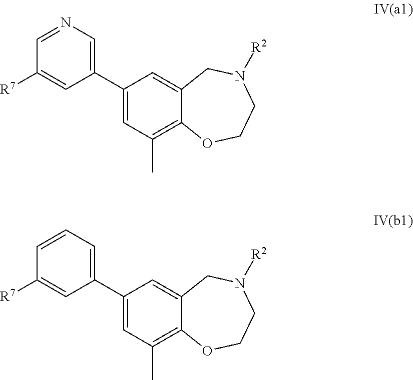
wherein R⁷ is —OH, —NH₂, —SO₂NH₂, —NHSO₂Me, or methoxy; and 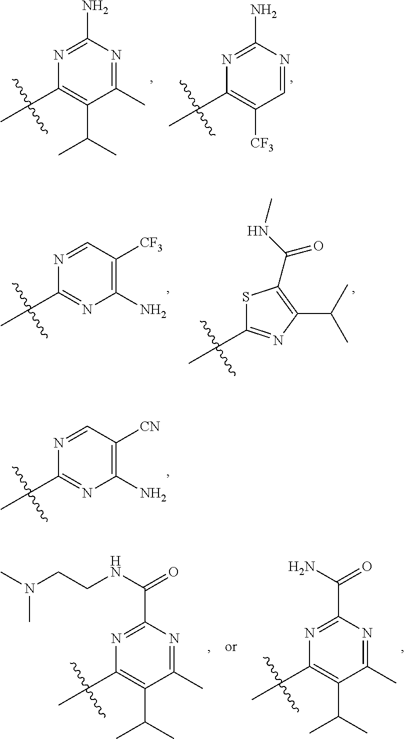
wherein R² is

35. Cancelled.

36. Cancelled.

37. The compound of claim 26, wherein the compound is a compound of Formula IV(a2) or IV(b2) 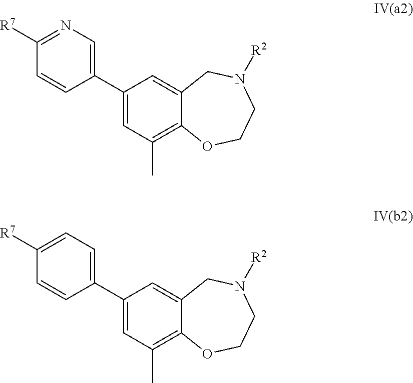
wherein R is NH₂, chloro, hydroxy, —CC₂Me, or methoxy; and 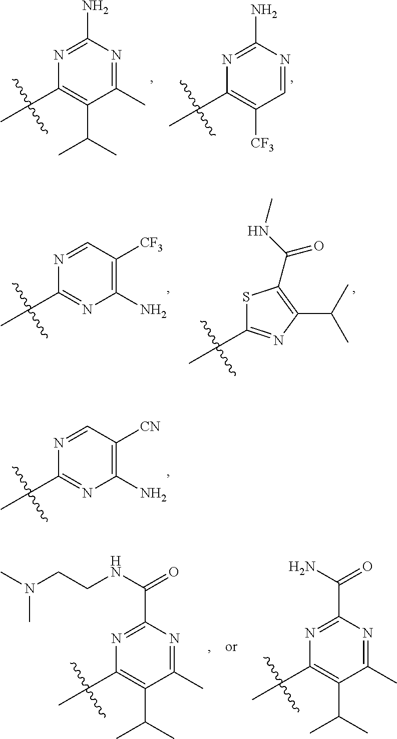
wherein R² is

38. Cancelled.

39. Cancelled.

40. The compound of claim 26, wherein the compound which is axompound of Formula IV(b3) 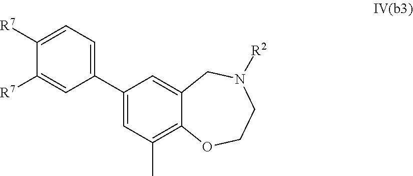 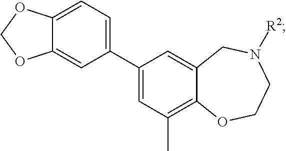
wherein the compound is and 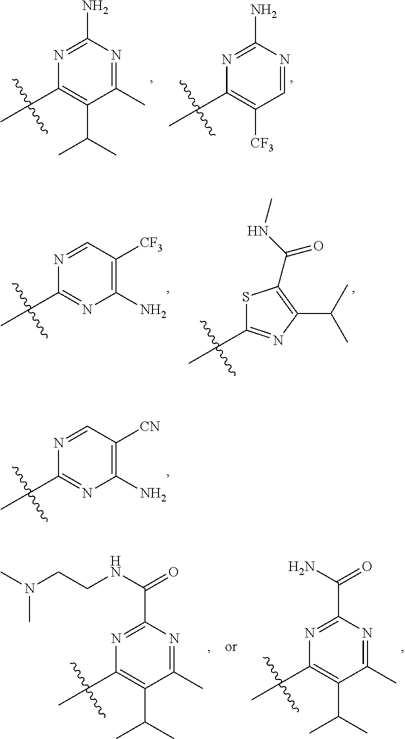
wherein R² is

41. Cancelled.

42. Cancelled.

43. The compound of claim 26, wherein the compound is a compound of Formula V(a), V(b), V(c), or V(d) 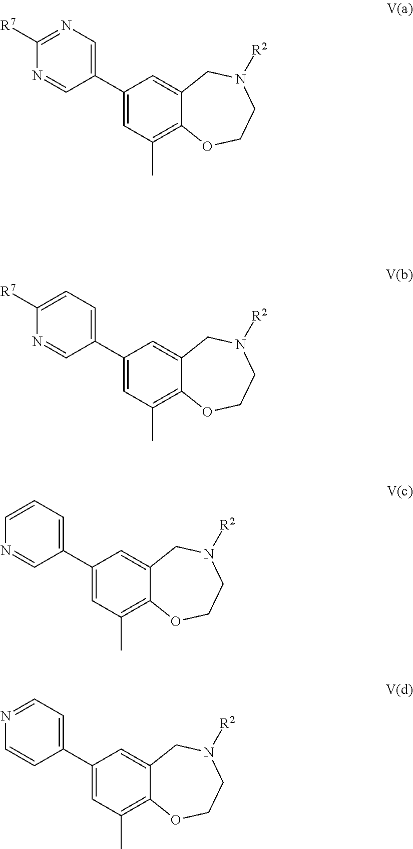
wherein R⁷ is fluoro, chloro, methoxy, NH2, chloro, hydroxy, —CC₂Me, or methoxy; and, 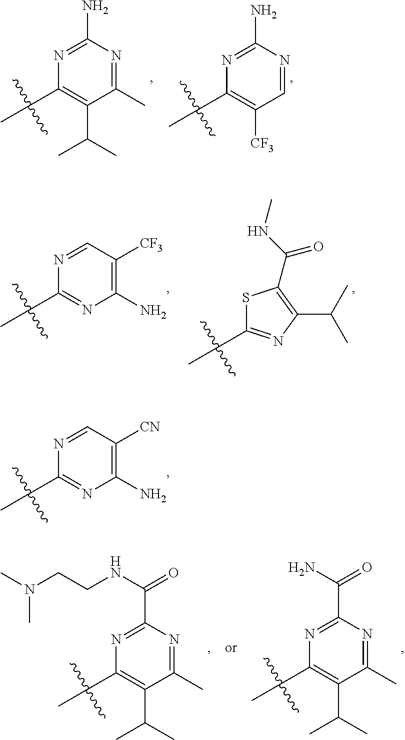
wherein and R² is

44. Cancelled.

45. The compound of claim 26, wherein the compound is a compound of Formula VI(a) or VI(b) 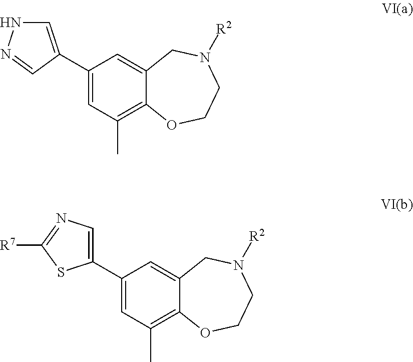
wherein R⁷ is fluoro, chloro, methoxy, NH₂, chloro, hydroxy, —CO₂Me, or methoxy; and 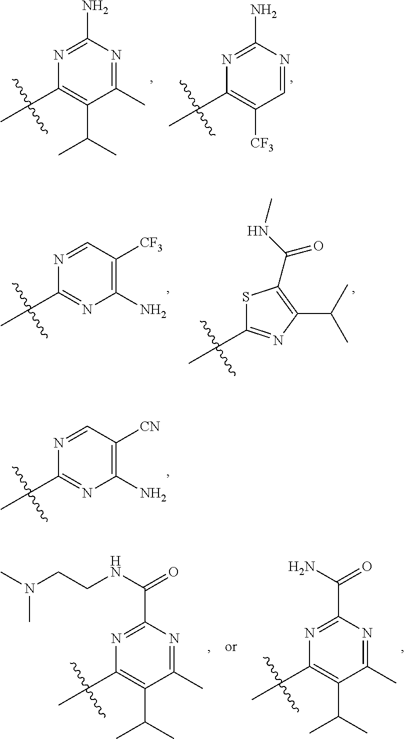
wherein R² is

46. Cancelled.

47. A compound of claim 1, which is:
4-methyl-5-(1-methylethyl)-6-[9-methyl-7-(2-methyl-1H-benzimidazol-6-yl)-2,3- dihydro-1,4-benzoxazepin-4(5H)-yl]pyrimidin-2-amine;
6-{4-[2-amino-6-methyl-5-(1-methylethyl)pyrimidin-4-yl]-9-methyl-2,3,4,5-tetrahydro- 1,4-benzoxazepin-7-yl}1,3]thiazolo[5,4-b]pyridin-2-amine;
2-amino-5-{4-[2-amino-6-methyl-5-(1-methylethyl)pyrimidin-4-yl]-9-methyl-2,3,4,5- tetrahydro-1,4-benzoxazepin-7-yl}pyridine-3-sulfonamide;
N-(5-{4-[2-amino-6-methyl-5-(1-methylethyl)pyrimidin-4-yl]-9-methyl-2,3,4,5- tetrahydro-1,4—benzoxazepin-7-yl}-2-chloropyridin-3-yl)methanesulfonamide;
6-[4-(2-amino-5,6-dimethylpyrimidin-4-yl)-9-methyl-2,3,4,5-tetrahydro-1,4benzoxazepin-7-yl][1,3]thiazolo[5,4-b]pyridin-2-amine;
6-[4-(2-amino-5-ethyl-6-methylpyrimidin-4-yl)-9-methyl-2,3,4,5-tetrahydro1,4benzoxazepin-7-yl][1,3]thiazolo[5,4-b]pyridin-2-amine;
6-[4-(2-amino5-ethenyl-6-methylpyrimidin-4-yl)-9-methyl-2,3,4,5-tetrahydro-1,4benzoxazepin-7-yl][1,3 thiazolo[5,4-b]pyridin-2—amine;
4-[7-(2-amino[1,3]thiazolo[5,4-b]pyridin-6-yl)-9-methyl-2,3-dihydro-1,4-benzoxazepin- 4(5H)-yl]-6,6-dimethyl-5,6,7,8-tetrahydroquinazolin-2-amine;
6-{4-[2-amino-5 -(1 -methylethyl)pyrimidin-4-yl]-9-methyl-2,3,4,5 -tetrahydro-1,4- benzoxazepin-7-yl}[1,3]thiazolo[5,4-b]pyridin-2-amine;
6-{4-[2-amino-5-(trifluoromethyl)pyrimidin-4-yl]-9-methyl-2,3,4,5-tetrahydro-1,4- benzoxazepin-7-yl}[1,3]thiazolo[5,4-b]pyridin-2-amine;
6-{4-[4-amino-5-(trifluoromethyl)pyrimidin-2-yl]-9-methyl-2,3,4,5-tetrahydro-1,4benzoxazepin-7- yl}- [1,3]thiazolo[5,4--b]pyridin-2-amine;
N-(5-{4-[2-amino-5-(trifluoromethyl)pyrimidin-4-yl]-9-methyl-2,3,4,5-tetrahydro-1,4benzoxazepin-7-yl}-2-chloropyridin-3-yl)methanesulfonamide;
N-(5-{4-[2-amino-5-(trifluoromethyl)pyrimidin-2-yl]-9-methyl-2,3,4,5-tetrahydro-1,4- benzoxazepin-7-yl}-2-chloropyridin-3-yl)methanesulfonamide;
6-(4-{2-amino-6-methyl-5 -[2-(methyloxy)ethyl]pyrimidin-4-yl}-9-methyl-2,3,4,5- tetrahydro-1,4-benzoxazepin-7-yl)[1,3 ]thiazolo[5,4-b]pyridin-2-amine;
6-{4-[2-amino-6-methyl-5-(1-methylethyl)pyrimidin-4-yl]-9-ethyl-2,3,4,5-tetrahydro- 1,4-benzoxazepin-7-yl}[1,3]thiazolo[5,4-b]pyridin-2—amine;
N-(5-{4-[2-amino-6-methyl-5(1-methylethyl)pyrimidin-4-yl]-9-methyl-2,3,4,5- tetrahydro-1,4-benzoxazepin—7-yl}pyridin-3-yl)methanesulfonamide;
N-(5-{4-[2-amino-6-methyl-5-(1-methylethyl)pyrimidin-4-]-9-methyl-2,3,4,5- tetrahydro-1,4-benzoxazepin-7-yl}-2-hydroxypyridin-3-yl)methanesulfonamide;
6--[9-methyl-4-(2,6,6-trimethyl-5,6,7,8-tetrahydroquinazolin-4—yl)-2,3,4,5-tetrahydro-1,4- benzoxazepin-7-yl]-[1,3]thiazolo[5,4-b]pyridin-2-amine;
N-[5-{4-[2-amino-6-methyl-5-(1-methylethyl)pyrimidin-4-yl]-9-methyl-2,3,4,5- tetrahydro-1,4-benzoxazepin-7-yl}-2-(methyloxy)pyridin-3 -yl]methanesulfonamide;
2-[7-{6-chloro-5-[(methylsulfonyl)amino]pyridin-3—yl} -9—methyl-2,3-dihydro-1,4- benzoxazepin-4(5H)-yl]-N-methyl-4-(1-methylethyl)-1,3-thiazole-5-carboxamide;
6--[4-(6,6-dimethyl-5,6-dihydroquinazolin-4-yl)-9-methyl-2,3,4,5-tetrahydro-1,4- benzoxazepin-7-yl][1,3]thiazolo[5,4-b]pyridin-2-amine;
4-[7-(6-aminopyridin-3-yl)-9-methyl-2,3-dihydro-1,4-benzoxazepin-4(5H)-yl]-6-methyl 5-(1-methylethyl)pyrimidin-2-amine;
6-[4-(4-aminopyrimidin-2-yl)-9-methyl-2,3,4,5-tetrahydro-1,4-benzoxazepin-7- yl][-1,3]thiazolo [5,4-b]pyridin-2—amine;
4-amino-2-[7-(2-amino[1,3]thiazolo[5,4-b]pyridin-6-yl)-9-methyl-2,3-dihydro-1,4- benzoxazepin-4(5H)-yl]pyrimidine-5-carbonitrile;
4-amino-2-[7-(2-amino[1,3]thiazolo[5,4-b]pyridin-6-yl)-9-methyl-2,3-dihydro-1,4- benzoxazepin-4(5H)-yl]pyrimidine-5-carboxamide;
N-{5-[4-(4-amino-5cyanopyrimidin-2-yl)-9-methyl-2,3,4,5—tetrahydro—l,4- benzoxazepin-7-yl]-2-chloropyridin-3-yl}methanesulfonamide;
6-{9-methyl-4-[(7S)-7-methyl-5,6,7,8-tetrahydroquinazolin-4-yl]-2,3,4,5-tetrahydro-1,4- benzoxazepin-7-yl}-[1,3]thiazolo[5,4-b]pyridin-2-amine;
6--(4-{2-[(dimethylamino)methyl]-6-methyl-5-(1-methylethyl)pyrimidin-4-yl}-9-methyl- 2,3,4,5-tetrahydro-1,4-benzoxazepin-7-yl)[1,3]thiazolo[5,4-b]pyridin—2—amine;
2-amino-6-[7-(2-amino[1,3]thiazolo[5,4-b]pyridin-6-yl)-9-methyl-2,3-dihydro-1,4- benzoxazepin-4(5H)-yl]pyridine-3,5-dicarbonitrile;
2-[7-{6-chloro-5-[(methylsulfonyl)amino]pyridin-3-yl}-9-methyl-2,3-dihydro-1,4- benzoxazepin-4(5H)-yl]-N-ethyl-4-(1-methylethyl)-1,3-thiazole-5-carboxamide;
3-7(2-amino[1,3]thiazolo[5,4-b]pyridin-6—yl)-9-methyl-2,3-dihydro-1,4-benzoxazepin- 4(5H)—yl]pyrazine-2-carbonitrile;
6-[4-(4-amino-5-fluoropyrimidin-2-yl)-9-methyl-2,3,4,5-tetrahydro-1,4-benzoxazepin-7- yl][1,3]thiazolo[5,4-b]pyridin-2-amine;
6-[7-(2-amino[1,3]thiazolo[5,4-b]pyridin-6-yl)-9-methyl-2,3-dihydro-1,4-benzoxazepin- 4(5H)-yl]pyridine-3-carbonitrile;
6-4-(4-amino-5-methylpyrimidin-2-yl)-9-methyl-2,3,4,5-tetrahydro-1,4-benzoxazepin-7- yl][1,3]thiazolo[5,4-b]pyridin-2-amine;
2-[7-(2-amino[1,3]thiazolo[5,4-b]pyridin-6-yl)-9-methyl-2,3 -dihydro-1,4-benzoxazepin- 4(5H)-yl]pyridine-3-carbonitrile;
2-[7-(2-amino[1,3]thiazolo[5,4-b]pyridin-6-yl)-9-methyl-2,3-dihydro-1,4-benzoxazepin- 4(5H)-yl]pyridine-3-carboxamide;
6-[4-(2-amino-6-chloro-5-ethenylpyrimidin-4-yl)-9-methyl-2,3,4,5-tetrahydro-1,4- benzoxazepin-13 7-yl][1,3]thiazolo[5,4-b]pyridin-2-amine;
6-[4-(2-amino-6-methyl-5-propylpyrimidin-4-yl)-9-methyl-2,3,4,5-tetrahydro-1,4- benzoxazepin-7-yl][1,3]thiazolo[5,4-b]pyridin-2-amine;
4-{7-[4-(1 H-imidazol-2-yl)phenyl]-9-methyl-2,3 -dihydro-1,4-benzoxazepin-4(5H)-yl}-6- methyl-5 -(1 -methylethyl)pyrimidin-2-amine;
4-[7-{6-chloro-5-[(methylsulfonyl)amino]pyridin-3 -yl}-9-methy1—2,3 -dihydro-1,4- benzoxazepin-4(5H)-yl]-N-[2-(dimethylamino)ethyl]-6-methyl-5-(1-methylethyl)pyrimidine-2- carboxamide;
4-[7-{6-chloro-5 -[(methylsulfonyl)amino]pyridin-3 -yl}-9-methy12,3-dihydro-1,4- benzoxazepin-4(5H)-yl]-6-methyl-5-(1-methylethyl)pyrimidine-2-carboxamide;
N,N-dimethyl-1 -{4-methyl-5-(1 -methylethyl)-6-[9-methyl-7—(2-methyl-1 H-benzimidazol- 6-yl)-2,3-dihydro-1,4-benzoxazepin-4(5H)-yl]pyrimidin-2-yl}methanamine;
6-{4-[2-amino-5-(cyclopropylmethyl)-6-methylpyrimidin-4-yl]-9-methyl-2,3,4,5tetrahydro-1,4-benzoxazepin-7-yl}[1,3]thiazolo[5,4-b]pyridin—2-amine;
4-(6-iodoquinazolin-4—yl)-9-methyl-7-(2-methyl-1H—benzimidazol-6-yl)-2,3,4,5- tetrahydro-1,4-benzoxazepine;
6-[9-methyl-4-(3-methylpyridin-4-yl)-2,3,4,5-tetrahydro-1,4—benzoxazepin-7- yl1,3 thiazolo[5,4-b]pyridin-2-amine;
6-{4-[2-amino-5-(3-fluorophenyl)-6-methylpyrimidin-4-yl]-9-methyl—2,3,4,5-tetrahydro- 1,4-benzoxazepin-7-yl}[1,3]thiazolo[5,4-b]pyridin-2-amine;
N′-{4-[7-(2-amino[1,3]thiazolo[5,4-b]pyridin-6-yl)-9-methyl-2,3-dihydro-4- benzoxazepin-4(5H)-yl]-6-methyl-5 -(1 -methylethyl)pyrimidin-2-yl}-N,N-dimethylethane-1,2- diamine;
6-[4-(2-amino-6-methyl-5-prop-2-en-1-ylpyrimidin-4-yl)-9-methyl-2,3,4,5-tetrahydro- 1,4-benzoxazepin-7-yl][1,3]thiazolo[5,4-b]pyridin-2-amine;
N-(5-{4-[2-amino-6-methyl-5-(1-methylethyl)pyrimidin-4-yl]-9-methyl-2,3,4,5- tetrahydro-1,4-benzoxazepin-7-yl}-2-chloropyridin-3 -yl)-1,1,1 -trifluoromethanesulfonamide;
6-{9-methyl-4-[6-methyl-2-(methylamino)-5-( 1 -methylethyl)pyrimidin-4-yl]-2,3,4,5- tetrahydro-1,4-benzoxazepin-7-yl}-[1,3]thiazolo[5,4-13 b]pyridin-2—amine;
6-[4-(2-amino-6-chloro-5-ethylpyrimidin-4-yl)-9-methyl-2,3,4,5-tetrahydro-1,4- benzoxazepin-7-yl][1,3]thiazolo [5,4-b]pyridin-2-amine;
6-amino-2-[7-(2-amino[1,3]thiazolo[5,4-b]pyridin-6-yl)-9-methyl-2,3-dihydro-1,4- benzoxazepin-4(5H)-yl]pyridine-3-arbonitrile;
6-{4-[2-amino-6-ethyl-5-(1-methylethyl)pyrimidin-4-yl]-9-methyl-13 2,3,4,5-tetrahydro- 1,4-benzoxazepin-7-yl}[1,3]thiazolo[5,4-b]pyridin-2-amine;
2-amino-6-[7-(2-amino[l,3]thiazolo[5,4-b]pyridin-6-yl)-9-methyl-2,3-dihydro1,4- benzoxazepin-4(5H)-yl]-4-methylpyridine-3,5-dicarbonitrile;
6-(4-{2-[(dimethylamino)methyl]-5-(1 -methylethyl)pyrimidin-4-yl}-9-methyl-2,3,4,5- tetrahydro-1,4-benzoxazepin-7-yl)[1,3]thiazolo[5,4-b]pyridin-2-amine;
6-{9-methyl-4-[6-methyl-5-(1 -methylethyl)-2-(pyrrolidin-1 -ylmethyl)pyrimidin-4-yl]- 2,3,4,5-tetrahydro-1,4-benzoxazepin-7-yl}[1,3]thiazolo[5,4-b]pyridin-2-amine;
6-(4-{2-[(dimethylamino)methyl]-5-(2,2,2-trifluoroethyl)pyrimidin-4-yl}-9-methyl- 2,3,4,5-tetrahydro-1,4-benzoxazepin-7-yl) [1,3]thiazolo [5,4-b]pyridin-2-amine;
6-(4-{6-chloro-2- [(dimethylamino)methyl]-5 -ethylpyrimidin-4-yl}-9-methyl-2,3,4,5- tetrahydro-1,4-benzoxazepin-7-yl)[1,3 thiazolo [5,4-b]pyridin-2-amine;
6-{4-[2-amino-6-chloro-5-(1-methylethyl)pyrimidin-4-yl]-9-methyl-2,3,4,5-tetrahydro- 1,4-benzoxazepin-7-yl}[1,3]thiazolo[5,4-b]pyridin-2-amine;
6-(4-{6-chloro-2-[(dimethylamino)methyl]-5-(1-methylethyl)pyrimidin-4-yl}-9-methyl- 2,3,4,5-tetrahydro-1,4-benzoxazepin-7-yl) [1,3]thiazolo [5,4-b]pyridin-2-amine;
{4-[7-(2-amino[1,3]thiazolo[5,4-b]pyridin-6-yl)-9-methyl-2,3-dihydro-1,4-benzoxazepin—4(5H)-yl]-6-methyl-5-(1-methylethyl)pyrimidin-2-yl}methanol;
6-(4-{2-[(diethylamino)methyl]-6-methyl-5-(1-methylethyl)pyrimidin-4-yl}-9-methyl- 2,3,4,5-tetrahydro-1,4-benzoxazepin-7-yl)[1,3]thiazolo[5,4-b]pyridin-2-amine;
6-(4-{2-[(dimethylamino)methyl]-5-ethylpyrimidin-4-yl}-9-methyl-2,3,4,5-tetrahydro- 1,4-benzoxazepin-7-yl)[1,3]thiazolo[5,4-b]pyridin-2-amine;
6-(41{2-[(dimethylamino)methyl]-5-ethyl-6-methylpyrimidin-4-yl}-9-methyl-2,3,4,5- tetrahydro-1,4-benzoxazepin-7-yl) [1,3]thiazolo [5,4-b]pyridin-2-amine;
methyl 4-{4-[2-amino-6-methyl-5-(1-methylethyl)pyrimidin-4-yl]-9-methyl-2,3,4,5- tetrahydro-1,4-benzoxazepin-7-yl}-2-(methyloxy)benzoate;
4-[7-(3-aminophenyl)-9-methyl-2,3-dihydro-1,4-benzoxazepin-4(5H)-yl]-6-methyl-5-(1- methylethyl)pyrimidin-2-amine;
3-{4-[2-amino-6-methyl-5-(1-methylethyl)pyrimidin-4-yl]-9—methyl-2,3,4,5-tetrahydro- 1,4-benzoxazepin-7-y 1}phenol;
4-methyl-5-(1-methylethyl)-6-(9-methyl-7-pyrimidin-5-yl-2,3-dihydro-1,4-benzoxazepin- 4(5H)-yl)pyrimidin-2-amine;
4-methyl-5-(1-methylethyl)-6-[9-methyl-7-(1H-pyrazol-5-yl)-2,3-dihydro-1,4- benzoxazepin-4(5H)-yl]pyrimidin-2-amine;
4-[7-(1,3-benzodioxol-5-yl)-9-methyl-2,3-dihydro-1,4-benzoxazepin-4(5H)-yl]-6-methyl- 5 -(1 -methylethyl)pyrimidin-2-amine;
4-methyl-5 -(1 -methylethyl)-6- {9-methyl-7- [6-(methyloxy)pyridin-3 -yl]-2,3 -dihydro-1,4- benzoxazepin-4(5H)-yl}pyrimidin-2-amine;
4-methyl-5-(1-methylethyl)-6-(9-methyl-7-pyridin-4-yl-2,3-dihydro-1,4-benzoxazepin- 4(5H)-yl)pyrimidin-2-amine;
4-methyl-5-(1-methylethyl)-6-(9-methyl-7-pyridin-3-yl-2,3-dihydro-1,4-benzoxazepin- 4(5 H)-yl)pyrimidin-2-amine;
3-{4-[2-amino-6-methyl-5-(1-methylethyl)pyrimidin-4-yl]-9-methyl-2,3,4,5-tetrahydro- 1,4-benzoxazepin-7-yl}benzamide;
4-{7-[3,4-bis(methyloxy)phenyl]-9-methyl-2,3-dihydro-1,4-benzoxazepin-4(5H)-yl}-6- methyl-5-(1-methylethyl)pyrimidin-2-amine;
4-methyl-5-(1-methylethyl)-6-{9-methyl-7-[5-(methyloxy)pyridin-3-yl]-2,3-dihydro-1,4- benzoxazepin-4(5H)-yl}pyrimidin-2-amine;
4-methyl-5-(1-methylethyl)-6-[9-methyl-7-(1H-pyrazol-4-yl)-2,3-dihydro-1,4- benzoxazepin-4(5H)-yl]pyrimidin-2-amine;
4-[7-(2-aminopyrimidin-5-yl)-9-methyl-2,3-dihydro-1,4-benzoxazepin-4(5H)-yl]-6- methyl-5-(1-methylethyl)pyrimidin-2-amine;
4-methyl-5-(1-methylethyl)-6-{9-methyl-7-[2-(methyloxy)pyrimidin-5-yl]-2,3-dihydro- 1,4-benzoxazepin-4(5H)-yl}pyrimidin-2-amine;
4-[7-(2-fluoropyridin-4-yl)-9-methyl-2,3-dihydro-1,4-benzoxazepin-4(5H)-yl]-6-methyl- 5-(1-methylethyl)pyrimidin-2-amine;
4-[7-(2-amino-1,3-thiazol-5-yl)-9-methyl-2,3-dihydro-1,4-benzoxazepin-4(5H)-yl]-6- methyl-5-(1-methylethyl)pyrimidin-2-amine;
6-(4-{2-[(dimethylamino)methyl]-5,6-diethylpyrimidin-4-yl}-9-methyl-2,3,4,5- tetrahydro-1,4-benzoxazepin-7-yl)[2,3]thiazolo[5,4-b]pyridin-2-amine;
6- {9-methyl-4- [6-methyl-5-( 1 -methylethyl)-2-(methylsulfonyl)pyrimidin-4-yl]-2,3,4,5- tetrahydro-1,4-benzoxazepin-7-yl}[1,3]thiazolo[5,4-b]pyridin-2-amine;
6- {9-methyl-4-[6-methyl-5-( 1 -methylethyl)pyrimidin-4-yl]-2,3,4,5-tetrahydro-1,4- benzoxazepin-7-yl}[1,3]thiazolo[5,4-b]pyridin-2-amine;
6-(4- {2- [(dimethylamino)methyl]-6-ethyl-5 -(1 -methylethyl)pyrimidin-4-yl}-9-methyl- 2,3,4,5-tetrahydro-1,4-benzoxazepin-7-yl)[1,3]thiazolo[5,4-b]pyridin-2-amine;
6-[4-(2-amino-5-ethenylpyrimidin-4-yl)-9-methyl-2,3,4,5-tetrahydro-1,4-benzoxazepin- 7-yl][1,3]thiazolo[5,4-b]pyridin-2-amine;
6-{4-[2-{[(1,1-dimethylethyl)amino]methyl}-6-methyl-5-(1-methylethyl)pyrimidin-4-yl]- 9-methyl-2,3,4,5-tetrahydro-1,4-benzoxazepin-7-yl}[1,3]thiazolo [5,4-b]pyridin-2-amine;
6-(4-{2-[(3,3 -difluoropyrrolidin-1 -yl)methyl]-6-methy 1,5 -(1 -methylethyl)pyrimidin-4- yl}-9-methyl-2,3,4,5-tetrahydro-1,4-benzoxazepin-7-yl)[1,3]thiazolo[5,4-b]pyridin-2-amine;
6- {9-methyl-4- [5 -(1 -methylethyl)-2-(pyrrolidin-1 -ylmethyl)pyrimidin-4-yl]-2,3,4,5- tetrahydro-1,4-benzoxazepin-7-yl}[1,3]thiazolo[5,4-b]pyridin-2-amine;
6-(9-methyl-4-{6-methyl-5-(1-methylethyl)-2-[(methyloxy)methyl]pyrimidin-4-yl}- 2,3,4,5-tetrahydro-1,4-benzoxazepin-7-yl)[1,3]thiazolo[5,4-b]pyridin-2-amine;
1-{4-[7-(2-amino[1,3]thiazolo[5,4-b]pyridin-6-yl)-9-methyl-2,3-dihydro-1,4- benzoxazepin-4(5H)-yl]-6-methyl-5-(1-methylethyl)pyrimidin-2-yl}-2,2,2-trifluoroethanol;
6-{9-methyl-4-[6-methyl-5-(1-methylethyl)-2-(morpholin-4-ylmethyl)pyrimidin-4-yl]- 2,3,4,5-tetrahydro-1,4-benzoxazepin-7-yl}[1,3]thiazolo[5,4-b]pyridin-2-amine; 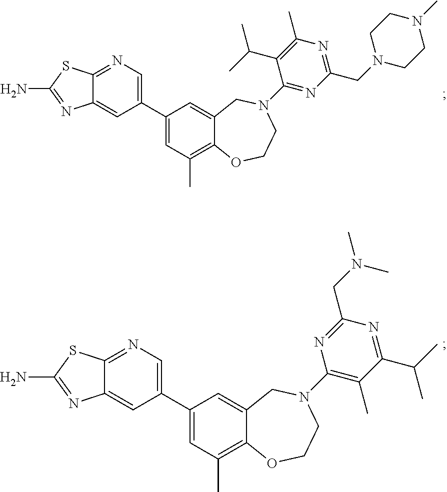 [SMILES: Cc1cc(-c2cnc3sc(N)nc3c2)cc2c1OCCN(c1nc(CN3CCN(C)CC3)nc(C)c1C(C)C)C2 ; Cc1cc(-c2cnc3sc(N)nc3c2)cc2c1OCCN(c1nc(CN(C)C)nc(C(C)C)c1C)C2]
1-{4-[7-(2-amino[1,3]thiazolo[5,4-b]pyridin-6-yl)-9-methyl-2,3-dihydro-1,4- benzoxazepin-4(5H)-yl]-6-methyl-5-(1-methylethyl)pyrimidin-2-yl}ethanol;
6-{9-methyl-4-[6-methyl-5-(1-methylethyl)-2-(methylsulfinyl)pyrimidin-4-yl]-2,3,4,5- tetrahydro-1,4-benzoxazepin-7-yl} [1,3]thiazolo[5,4-b]pyridin-2-amine;
6-{4-[2-{[(1,1 -dimethylethyl)(methyl)amino]methyl}-6-methyl-5-(1- methylethyl)pyrimidin-4-yl]-9-methyl-2,3,4,5-tetrahydro-1,4-benzoxazepin-7- yl}[1,3]thiazolo[5,4-b]pyridin-2-amine;
6-{4-[2-{[(2,2-difluoroethyl)amino]methyl}-6-methyl-5-(1-methylethyl)pyrimidin-4-yl]- 9-methyl-2,3,4,5-tetrahydro-1,4-benzoxazepin-7-yl}[1,3]thiazolo[5,4-b]pyridin-2-amine;
6—{9-methyl-4-[6-methyl-5-( 1 -methylethyl)-2-(4-methylpiperazin-1 -yl)pyrimidin-4-yl]- 2,3,4,5-tetrahydro-1,4-benzoxazepin-7-yl}[1,3]thiazolo[5,4-b]pyridin-2-amine;
6-{9-methyl-4-[6-methyl-5-(1-methylethyl)-2-{[(2,2,2- trifluoroethyl)amino]methyl}pyrimidin-4-yl]-2,3,4,5-tetrahydro-1,4-benzoxazepin-7- yl}[1,3]thiazolo[5,4-b]pyridin-2-amine;
6-{4-[2,6-dimethyl-5-(1-methylethyl)pyrimidin-4-yl]-9-methyl-2,3,4,5-tetrahydro-1,4- benzoxazepin-7-yl}[1,3]thiazolo[5,4-b]pyridin-2-amine;
{4-[7-(2-amino[1,3]thiazolo[5,4-b]pyridin-6-yl)-9-methyl-2,3-dihydro-1,4-benzoxazepin- 4(5H)-yl]-6-methyl-5-(1-methylethyl)pyrimidin-2-yl}acetonitrile;
N-(5-{4-[2-amino-6-methyl-5-(1-methylethyl)pyrimidin-4-yl]-9-methyl-2,3,4,5- tetrahydro-1,4-benzoxazepin-7-yl}-1,3-thiazol-2-yl)acetamide;
6-{9-methyl-4-[2-methyl-5-(1-methylethyl)pyrimidin-4-yl]-2,3,4,5-tetrahydro-1,4- benzoxazepin-7-yl}[1,3]thiazolo[5,4-b]pyridin-2-amine;
6-{4-[6-chloro-5-(1-methylethyl)pyrimidin-4-yl]-9-methyl-2,3,4,5-tetrahydro-1,4- benzoxazepin-7-yl}[1,3]thiazolo[5,4-b]pyridin-2-amine;
4-[7-( 1,3 -dimethyl-1H-pyrazol-4-yl)-9-methyl-2,3 -dihydro-1,4-benzoxazepin-4(5H)-yl]- 6-methyl-5-(1-methylethyl)pyrimidin-2-amine;
4-[7-(1,5-dimethyl-1H-pyrazol-4-yl)-9-methyl-2,3-dihydro-1,4-benzoxazepin-4(5H)-yl]- 6-methyl-5-(1-methylethyl)pyrimidin-2-amine;
4-[7-(1-ethyl-1H-pyrazol-4-yl)-9-methyl-2,3 -dihydro-1,4-benzoxazepin-4(5H)-yl]-6- methyl-5-(1-methylethyl)pyrimidin-2-amine;
4-methyl-5-(1-methylethyl)-6-{9-methyl-7-[2-(methylamino)-1,3-thiazol-4-yl]-2,3- dihydro-1,4-benzoxazepin-4(5H)-yl}pyrimidin-2-amine;
4-[7-(2-amino[1,3]thiazolo[5,4-b]pyridin-6-yl)-9-methyl-2,3-dihydro-1,4-benzoxazepin- 4(5H)-yl]-N-ethyl-6-methyl-5-(1-methylethyl)pyrimidine-2-carboxamide;
2-{4-[7-(2-amino[1,3]thiazolo[5,4-b]pyridin-6-yl)-9-methyl-2,3-dihydro-1,4- , benzoxazepin-4(5H)-yl]-6-chloro-2-(methylthio)pyrimidin-5-yl}propan-2-ol;
6-[4-(5-ethenyl-6-methylpyrimidin-4-yl)-9-methyl-2,3,4,5-tetrahydro-1,4-benzoxazepin- 7-yl][1,3]thiazolo[5,4-b]pyridin-2-amine;
6- {9-methyl4- [5 -(1 -methylethy l)pyrimidin-4-yl]-2,3,4,5 -tetrahydro-1,4-benzoxazepin-7- yl}[1,3]thiazolo[5,4-b]pyridin-2-amine;
4-methvl-5-(1-methylethyl)-6-[9-methyl-7-(1-methyl-1H-pyrazol-4-yl)-2,3-dihydro-1,4- benzoxazepin-4(5H)-yl]pyrimidin-2-amine;
4-methyl-5-(1-methylethyl)-6-[9-methyl-7-(2-methyl-1,3-thiazol-5-yl)-2,3-dihydro-1,4- benzoxazepin-4(5H)-yl]pyrimidin-2-amine;
N-({4-[7-(2-amino[1,3]thiazolo[5,4-b]pyridin-6-yl)-9-methyl-2,3-dihydro-1,4- benzoxazepin-4(5H)-yl]-6-methyl-5-(1-methylethyl)pyrimidin-2-yl}methyl)acetamide;
6-{4-[2-(fluoromethyl)-6-methyl-5-(1-methylethyl)pyrimidin-4-yl]-9-methyl-2,3,4,5- tetrahydro-1,4-benzoxazepin-7-yl}[1,3]thiazolo[5,4-b]pyridin-2-amine;
6-(4- {2-[(cyclopropylamino)methyl]-6-methyl-5-(1 -methylethyl)pyrimidin-4-yl}-9- methyl-2,3,4,5-tetrahydro-1,4-benzoxazepin-7-yl)[1,3]thazolo[5,41b]pyridin-2-amine;
6-{4-[2-amino-6-methyl-5-(1-methylethyl)pyrimidin-4-yl]-2,3,4,5-tetrahydro-1,4- benzoxazepin-7-yl}[1,3]thiazolo[5,4-b]pyridin-2-amine;-d₄
6-{4-[2-amino-6-methyl-5-(1-methylethyl)pyrimidin-4-yl]-2,3,4,5-tetrahydro-1,4- benzoxazepin-7-yl}[1,3]thiazolo[5,4-b]pyridin-2-amine;-d₆
6-{9-methyl-4-[6-methyl-5-(1-methylethenyl)pyrimidin-4-yl]-2,3,4,5-tetrahydro-1,4- benzoxazepin-7-yl}[1,3]thiazolo[5,4-b]pyridin-2-amine;
1-{4-[7-(2-amino[1,3]thiazolo[5,4-b]pyridin-6-yl)-9-methyl-2,3-dihydro-1,4- benzoxazepin-4(5H)-yl]-6-methylpyrimidin-5-yl}ethanone;
6-{4-[2-{[(2-fluoroethyl)amino]methyl}-6-methyl-5-(1-methylethyl)pyrimidin-4-yl]-9- methyl-2,3,4,5-tetrahydro-1,4-benzoxazepin-7-yl}[1,3]thiazolo[5,4-b]pyridin-2-amine;
6-(9-methyl-4-{6-methyl-5-[2-(methyloxy)ethyl]-2-(pyrrolidin-1-ylmethyl)pyrimidin-4- yl}-2,3,4,5-tetrahydro-1,4-benzoxazepin-7-yl)[1,3]thiazolo[5,4-b]pyridin-2-amine;
6-{9-methyl-4-[6-methyl-5-(1-methylethyl)-2-(trifluoromethyl)pyrimidin-4-yl]-2,3,4,5- tetrahydro-1,4-benzoxazepin-7-yl}[1,3]thiazolo[5,4-b]pyridin-2-amine;
6-(9-methyl-4-{6-methyl-5-[2-(methyloxy)ethyl]pyrimidin-4-yl}-2,3,4,5-tetrahydro-1,4- benzoxazepin-7-yl)[1,3]thiazolo[5,4-b]pyridin-2-amine;
6-{4-[2-amino-6-methyl-5-(1-methylethenyl)pyrimidin-4-yl]-9-methyl-2,3,4,5- tetrahydro- 1,4-benzoxazepin-7-yl}[ 1,3 ]thiazolo [5,4-b]pyridin-2-amine; a
2-{4-[7-(2-amino[1,3]thiazolo[5,4-b]pyridin-6-yl)-9-methyl-2,3-dihydro-1,4- benzoxazepin-4(5H)-yl]-6-chloropyrimidin-5-yl}propan-2-ol;
6-(4- {2,6-dimethyl-5-[2-(methyloxy)ethyl]pyrimidin-4-yl}-9-methyl-2,3,4,5-tetrahydro- 1,4-benzoxazepin-7-yl) [1,3]thiazolo [5,4-b]pyridin-2-amine;
6-{4-[2-azetidin-3-yl-6-methyl-5-(1-methylethyl)pyrimidin-4-yl]-9-methyl-2,3,4,5- tetrahydro-1,4-benzoxazepin-7-yl}[1,3]thiazolo[5,4-b]pyridin-2-amine;
6-{4-[2-(aminomethyl)-6-methyl-5-(1-methylethyl)pyrimidin-4-yl]-9-methyl-2,3,4,5- tetrahydro-1,4-benzoxazepin-7-yl}[1,3]thiazolo[5,4-b]pyridin-2-amine;
6-(9-methyl-4-{2-methyl-5-[2-(methyloxy)ethyl]pyrimidin-4-yl}-2,3,4,5-tetrahydro-1,4- benzoxazepin-7-yl) [1,3]thiazolo [5,4-b]pyridin-2-amine;
6-(9-methyl-4- {6-methyl-2- [(methylamino)methyl]-5 -(1 -methylethy l)pyrimidin-4-yl}- 2,3,4,5-tetrahydro-1,4-benzoxazepin-7-yl) [1,3]thiazolo [5,4-b]pyridin-2-amine;
4-[7-(5-amino-1,3,4-thiadiazol-2-yl)-9-methyl-2,3-dihydro-1,4-benzoxazepin-4(5H)-yl]- 6-methyl-5-(1 -methylethyl)pyrimidin-2-amine;
6-[4-(2,6-dimethyl-5-prop-2-yn-1-ylpyrimidin-4-yl)-9-methyl-2,3,4,5-tetrahydro-1,4- benzoxazepin-7-yl][1,3]thiazolo [5,4-b]pyridin-2-amine;
1-{4-[7-(2-amino[1,3]thiazolo[5,4-b]pyridin-6-yl)-9-methyl-2,3-dihydro-1,4- benzoxazepin-4(5 H)-yl]-6-methyl-5 -(1 -methylethyl)pyrimidin-2-yl}azetidin—3-ol;
6-{4-[2-amino-6-methyl-5-(1-methylethyl)pyrimidin-4-yl]-9-(methyloxy)-2,3,4,5- tetrahydro-1,4-benzoxazepin-7-y 1} [1,3 jthiazo lo [5,4-b]pyridin-2-amine;
6-[4-(5-but-2-yn-1-yl-2,6-dimethylpyrimidin-4-yl)-9-methyl-2,3,4,5-tetrahydro-1,4- benzoxazepin-7-yl][1,3]thiazolo[5,4-b]pyridin-2-amine;
6-(4-{2,6-dimethyl-5-[1-(methyloxy)ethyl]pyrimidin-4-yl}-9-methyl-2,3,4,5-tetrahydro- 1,4-benzoxazepin-7-yl)[1,3]thiazolo[5,4-b]pyridin-2-amine;
6-(4- {2,6-dimethyl-5- [(methyloxy)methyl]pyrimidin-4-yl}-9-methyl-2,3,4,5-tetrahydro- 1,4-benzoxazepin-7-yl)[1,3]thiazolo[5,4-b]pyridin-2-amine;
6-{4-[2-(difluoromethyl)-6-methyl-5-(1-methylethyl)pyrimidin-4-yl]-9-methyl-2,3,4,5- tetrahydro-1,4-benzoxazepin-7-yl}[1,3]thiazolo[5,4-b]pyridin-2-amine;
6-[4-(2-amino-5-ethynyl-6-methylpyrimidin-4-yl)-9-methyl-2,3,4,5-tetrahydro-1,4- benzoxazepin-7-yl][1,3]thiazolo[5,4-b]pyridin-2-amine;
6{-9-methyl-4-[6-methyl-5-(1-methylethyl)-2-pyrrolidin-2-ylpyrimidin-4-yl]-2,3,4,5- tetrahydro-1,4-benzoxazepin-7-yl}[1,3thiazo lo [5,4-b]pyridin-2-amine;
6-(4-{2-[(2S)-4,4-difluoropyrrolidin-2-yl]-6-methyl-5-(1-methylethyl)pyrimidin-4-yl}-9- methyl-2,3,4,5-tetrahydro-1,4-benzoxazepin-7-yl)[1,3]thiazolo[5,4-b]pyridin-2-amine;
6-{9-methyl-4-[6-(methylamino)-5-nitropyrimidin-4-yl]-2,3,4,5-tetrahydro-1,4- benzoxazepin-7-yl}[1,3]thiazolo[5,4-b]pyridin-2-amine;
6-{9-methyl-4-[6-methyl-5-(1-methylethyl)-2-(1-methylpyrrolidin-2-yl)pyrimidin-4-yl]- 2,3,4,5-tetrahydro-1,4-benzoxazepin-7-yl}[1,3]thiazolo[5,4-b]pyridin-2-amine;
6-{4-[2-cyclopropyl-6-methyl-5-(1-methylethyl)pyrimidin-4-yl]-9-methyl-2,3,4,5- tetrahydro-1,4-benzoxazepin-7-yl}[1,3]thiazolo[5,4-b]pyridin-2-amine;
6-(4-{2-[(2S,4R)-4-fluoropyrrolidin-2-yl]-6-methyl-5-(1-methylethyl)pyrimidin-4-yl}-9- methyl-2,3,4,5-tetrahydro-1,4-benzoxazepin-7-yl)[1,3]thiazolo[5,4-b]pyridin-2-amine;
6-{9-methyl-4-[6-methyl-5-(1-methylethyl)-2-(methyloxy)pyrimidin-4-yl]-2,3,4,5- tetrahydro-1,4-benzoxazepin-7-yl}[1,3]thiazolo[5,4-b]pyridin-2-amine;
6-(4-{2,6-dimethyl-5-[1-methyl-2-(methyloxy)ethyl]pyrimidin-4-yl}-9-methyl-2,3,4,5- tetrahydro-1,4-benzoxazepin-7-yl)[1,3]thiazolo [5,4-b]pyridin-2-amine;
6- {9-methyl-4-[6-methyl-5-(1 -methylethyl)-2- {[2-(methyloxy)ethyl]oxy }pyrimidin-4- yl]-2,3,4,5-tetrahydro-1,4-benzoxazepin-7-yl}[1,3]thiazolo[5,4-b]pyridin-2-amine;
6-(9-methyl-4- {6-methyl-5-(1 -methylethyl)-2- [2-(methyloxy)ethyl]pyrimidin-4-yl}- 2,3,4,5-tetrahydro-1,4-benzoxazepin-7-yl) [1,3 ]thiazolo [5,4-b]pyridin-2-amine;
6- {4-[2- {[(2-fluoroethyl)(methyl)amino]methyl}-6-methyl-5 -(1 -methylethyl)pyrimidin- 4-yl]-9-methyl-2,3,4,5-tetrahydro-1,4-berizoxazepin-7-yl}[l,3]thiazolo[5,4-b]pyridin-2-amine;
6- [4- {2- [(dimethy lamino)methyl]-6-methyl5 -(1 -methylethyl)pyrimidin-4-yl}-9- (methyloxy)-2,3,4,5-tetrahydro-1,4-benzoxazepin-7-yl][1,3]thiazolo[5,4-b]pyridin-2-amine;
6-(4-{2-[(ethylamino)methyl]-6-methyl-5-(1-methylethyl)pyrimidin-4-yl}-9-methyl- 2,3,4,5-tetrahydro-1,4-benzoxazepin-7-yl)[1,3]thiazolo[5,4-b]pyridin-2-amine;
6- {4- [2- {[ethyl(2-fluoroethyl)amino]methyl}-6-methyl-5 -(1 -methylethyl)pyrimidin-4- yl]-9-methyl-2,3,4,5-tetrahydro-1,4-benzoxazepin-7-yl}[1,3]thiazolo[5,4-b]pyridin-2-amine;
N-[2-chloro-5-(9-methyl-4-{6-methyl-5-(1-methylethyl)-2-[2- (methyloxy)ethyl]pyrimidin-4-yl}-2,3,4,5-tetrahydro-1,4-benzoxazepin-7-yl)pyridin-3yl]methanesulfonamide;
N-(2-chloro-5-{4-[2-{[(2-fluoroethyl)amino]methyl}-6-methyl-5-(1- methylethyl)pyrimidin-4-yl]-9-methyl-2,3,4,5-tetrahydro-1,4-benzoxazepin-7-yl}pyridin-3- yl)methanesulfonamide;
4-methyl-5-(1-methylethyl)-6-[9-methyl-7-(2-methyl-3H-imidazo[4,5-b]pyridin-6-yl)- 2,3-dihydro-1,4-benzoxazepin-4(5H)-yl]pyrimidin-2-amine;
4-[7-(lH-imidazo[4,5-b]pyridin-6-yl)-9-methyl-2,3-dihydro-l,4-benzoxazepin-4(5H)-yl]- 6-methyl-5-( 1 -methylethyl)pyrimidin-2-amine;
N-(2-chloro-5-{4-[2-{[(2,2-difluoroethyl)amino]methyl}-6-methyl-5-(l- methylethyl)pyrimidin-4-yl]-9-methyl-2,3,4,5-tetrahydro-l,4-benzoxazepin-7-yl}pyridin-3- yl)methanesulfonamide;
2,2-difluoro-N-({4-[7-(lH-imidazo[4,5-b]pyridin-6-yl)-9-methyl-2,3-dihydro-l,4- benzoxazepin-4(5H)-yl]-6-methyl-5-(l-methylethyl)pyrimidin-2-yl}methyl)ethanamine;
2,2-difluoro-N-({4-methyl-5-(1-methylethyl)-6-[9-methyl-7-(2-methyl-1H-imidazo[4,5- b]pyridin-6-yl)-2,3 -dihydro-1,4-benzoxazepin-4(5H)-yl]pyrimidin-2-yl}methyl)ethanamine;
2,2-difluoro-N-({4-[7-(1H-imidazo[4,5-b]pyridin-6-yl)-9-methyl-2,3-dihydro-l,4- benzoxazepin-4(5H)-yl]-6-methyl-5-(1-methylethyl)pyrimidin-2-yl}methyl)-N- methylethanamine;
5- {4-[2- {[(2,2-difluoroethyl)(methyl)amino]methyl}-6-methyl-5-(1- methylethyl)pyrimidin-4-yl]-9-methy 1-2,3,4,5-tetrahydro-1,4-benzoxazepin-7-yl} -1,3,4- thiadiazol-2-amine;
5-{4-[2-{[(2,2-difluoroethyl)amino]methyl}-6-methyl-5-(1-methylethyl)pyrimidin-4-yl]- 9-methyl-2,3,4,5-tetrahydro-1,4-benzoxazepin-7-yl}-1,3,4-thiadiazol-2-amine;
5 - {4- [2- {[(2,2-difluoroethyl)(ethyl)amino]methyl}-6-methy 1-5 -(1 - methylethyl)pyrimidin-4-yl]-9-methyl-2,3,4,5-tetrahydro-l,4-benzoxazepin-7-yl}-l,3,4- thiadiazol-2-amine;
N-ethyl-2,2-difluoro-N-( {4- [7-(1H-imidazo[4,5 -b]pyridin-6-yl)-9-methyl-2,3 -dihydro- l,4-benzoxazepin-4(5H)-yl]-6-methyl-5-(1-methylethyl)pyrimidin-2-yl}methyl)ethanamine;
5-{4-[2,6-dimethyl-5-(1-methylethyl)pyrimidin-4-yl]-9-methyl-2,3,4,5-tetrahydro-l,4- benzoxazepin-7-yl}-1,3,4-thiadiazol-2-amine;
5-{9-methyl-4-[6-methyl-5-(1-methylethyl)pyrimidin-4-yl]-2,3,4,5-tetrahydro-l,4- benzoxazepin-7-yl}-1,3,4-thiadiazol-2-amine;
5-[4-(2,5-dimethylpyrimidin-4-yl)-9-methyl-2,3,4,5-tetrahydro-1,4-benzoxazepin-7-yl]- 1,3,4-thiadiazol-2-amine;
5-{9-methyl-4-[2-methyl-5-(1-methylethyl)pyrimidin-4-yl]-2,3,4,5-tetrahydro-1,4- benzoxazepin-7-yl}-1,3,4-thiadiazol-2-amine;
5-[4-(5,6-dimethylpyrimidin-4-yl)-9-methyl-2,3,4,5-tetrahydro-1,4-benzoxazepin-7-yl]- 1,3,4-thiadiazol-2-amine;
5-{9-methyl-4-[5-(1-methylethyl)pyrimidin-4-yl]-2,3,4,5-tetrahydro-1,4-benzoxazepin-7- yl}-1,3,4-thiadiazol-2-amine;
4-[7-(5-amino-l,3,4-thiadiazol-2-yl)-9-methyl-2,3-dihydro-l,4-benzoxazepin-4(5H)-yl]- 5-methylpyrimidin-2-amine;
4[-7-(5-amino-1,3,4-thiadiazol-2-yl)-9-methyl-2,3-dihydro-1,4-benzoxazepin-4(5H)-yl]- 5,6-dimethylpyrimidin-2-amine;
4-[7-(5-amino-l,3,4-thiadiazol-2-yl)-9-methyl-2,3-dihydro-1,4-benzoxazepin-4(5H)-yl]- 5-(1-methylethyl)pyrimidin-2-amine;
4-[7-(5-amino-1,3,4-thiadiazol-2-yl)-9-methyl-2,3-dihydro-1,4-benzoxazepin-4(5H)-yl]- 5-ethenyl-6-methylpyrimidin-2-amine; and
6- {4- [2-( 1 -aminoethyl)-6-methyl-5 -(1 -methylethyl)pyrimidin-4-yl]-9-methyl-2,3,4,5- tetrahydro-1,4-benzoxazepin-7-yl}[1,3]thiazolo[5,4-b]pyridin-2-amine
or a single stereoisomer or mixture of stereoisomers thereof and additionally optionally as a pharmaceutically acceptable salt thereof.

48. Cancelled.

49. A pharmaceutical composition which comprises a compound, optionally as pharmaceutically acceptable salt thereof, of any of claim 1 and a pharmaceutically acceptable carrier, excipient, or diluent.

50. A method of making a Compound of Formula I, according to claim 1, which method comprises
(a) reacting the following, or a salt thereof: 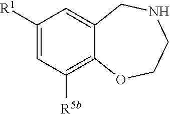
where R¹ is as defined in claim 1; with an intermediate of formula R²X where X is halo, and R² is as defined in claim 1 to yield a compound of Formula I; and optionally separating individual isomers; and optionally modifying any of the R¹ and R² groups; and optionally forming a pharmaceutically acceptable salt thereof; or
(b) reacting the following intermediate, or a salt thereof: 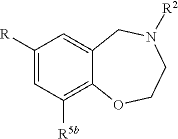
where R is halo or -B(OR′)₂ (where both R′ are hydrogen or the two R′ together form a boronic ester), and R² is as defined in claim ; with an intermediate of formula R¹Y where Y is halo when R is —B(OR′)₂ and Y is —B(OR′)₂ when R is halo, and R² is as defined in claim 1 to yield a compound of Formula I; and optionally separating individual isomers; and optionally modifying any of the R¹ and R² groups; and optionally forming a pharmaceutically acceptable salt, hydrate, solvate or combination thereof.

51. A method for treating cancer, the method comprising administering to a patient a therapeutically effective amount of a compound of claim 1, optionally as a pharmaceutically acceptable salt thereof, or a pharmaceutical composition comprising a compound of claim 1 and a pharmaceutically acceptable carrier, excipient, or diluent.

52. Cancelled.

53. The method of claim 51 where the cancer is breast cancer, mantle cell lymphoma, renal cell carcinoma, acute myelogenous leukemia, chronic myelogenous leukemia, NPM/ALK-transformed anaplastic large cell lymphoma, diffuse large B cell lymphoma, rhabdomyosarcoma, ovarian cancer, endometrial cancer, cervical cancer, non small cell lung carcinoma, small cell lung carcinoma, adenocarcinoma, colon cancer, rectal cancer, gastric carcinoma, hepatocellular carcinoma, melanoma, pancreatic cancer, prostate carcinoma, thyroid carcinoma, anaplastic large cell lymphoma, hemangioma, glioblastoma, or head and neck cancer.


SEQUENCE LISTING (8 sequences)

SEQ ID NO: 1 - protein, 1068 aa, Homo sapiens
  MISC_FEATURE: Human phosphatidylinositol 3-kinase catalytic subunit alpha polypeptide (PIK3CA) encoded by mRNA polynucleotide sequence ID NCBI Accession Reference No NM_006218; GI54792081 (3724 nucleotides)
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - DNA, 3724 bp, Homo sapiens
  misc_feature: Human phosphatidylinositol 3-kinase catalytic subunit alpha mRNA polynucleotide sequence ID NCBI Accession Reference No NM_006218; GI54792081 (3724 nucleotides)
tctccctcggcgccgccgccgccgcccgcggggctgggacccgatgcggttagagccgcggagcctggaagagccccgagcgtttctgctttgggacaaccatacatctaattccttaaagtagttttatatgtaaaacttgcaaagaatcagaacaatgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaaagataactgagaaaatgaaagctcactctggattccacactgcactgttaataactctcagcaggcaaagaccgattgcataggaattgcacaatccatgaacagcattagaatttacagcaagaacagaaataaaatactatataatttaaataatgtaaacgcaaacagggtttgatagcacttaaactagttcatttcaaaattaagctttagaataatgcgcaatttcatgttatgccttaagtccaaaaaggtaaactttgaagattgtttgtatctttttttaaaaaacaaaacaaaacaaaaatccccaaaatatatagaaatgatggagaaggaaaaaaaaaaaaaaaaa

SEQ ID NO: 3 - DNA, 23 bp, Artificial Sequence
  human exon 9 forward primer
gggaaaaatatgacaaagaaagc

SEQ ID NO: 4 - DNA, 22 bp, Artificial Sequence
  human exon 9 reverse primer
ctgagatcagccaaattcagtt

SEQ ID NO: 5 - DNA, 27 bp, Artificial Sequence
  human exon 9 sequencing primer
tagctagagacaatgaattaagggaaa

SEQ ID NO: 6 - DNA, 20 bp, Artificial Sequence
  human exon 20 forward primer
ctcaatgatgcttggctctg

SEQ ID NO: 7 - DNA, 21 bp, Artificial Sequence
  human exon 20 reverse primers
tggaatccagagtgagctttc

SEQ ID NO: 8 - DNA, 22 bp, Artificial Sequence
  human exon 20 sequencing primer
ttgatgacattgcatacattcg